(19) United States
(12) Patent Application Publication
(10) Pub. No.: US 2025/0122160 A1
(43) Pub. Date: Apr. 17, 2025

(54) PROTEIN TYROSINE PHOSPHATASE INHIBITORS AND METHODS OF USE THEREOF

(71) Applicants: Calico Life Sciences LLC, South San Francisco, CA (US); AbbVie Inc., North Chicago, IL (US)

(72) Inventors: Philip R. Kym, Libertyville, IL (US); Jennifer M. Frost, Gurnee, IL (US); Christos Economou, Wilmette, IL (US); Andrew Bogdan, Evanston, IL (US); Stacy Fosu, Lake Bluff, IL (US); Zhaoming Xiong, Cambridge, MA (US); Spencer O. Scholz, San Francisco, CA (US); Eric Allan Voight, Pleasant Prairie, WI (US); Elliot P. Farney, Evanston, IL (US); Mark A. Matulenko, Libertyville, IL (US); Matthew J. O'Connor, Chicago, IL (US); Christina Baumgartner, Lake Geneva, WI (US); Qingwei Zhang, Libertyville, IL (US); Reza Shiroodi, Mundelein, IL (US); Jason R. Abbott, Vernon Hills, IL (US); Geoff T. Halvorsen, Lake Bluff, IL (US)

(21) Appl. No.: 18/647,757

(22) Filed: Apr. 26, 2024

Related U.S. Application Data
(63) Continuation of application No. 18/044,706, filed on Jan. 1, 0001.
(60) Provisional application No. 63/077,330, filed on Sep. 11, 2020.

(51) Int. Cl. C07D 285/06; A61K 31/433; A61K 31/4439; A61K 31/454; A61K 31/4725; A61K 31/501; C07D 417/04; C07D 417/12; C07D 417/14

(52) U.S. Cl. CPC C07D 285/06; A61K 31/433; A61K 31/4439; A61K 31/454; A61K 31/4725; A61K 31/501; C07D 417/04; C07D 417/12; C07D 417/14

(57) ABSTRACT

Provided herein are compounds, compositions, and methods useful for inhibiting protein tyrosine phosphatase, e.g., protein tyrosine phosphatase non-receptor type 2 (PTPN2) and/or protein tyrosine phosphatase non-receptor type 1 (PTPN1), and for treating related diseases, disorders and conditions favorably responsive to PTPN1 or PTPN2 inhibitor treatment, e.g., a cancer or a metabolic disease.

### CROSS-REFERENCE TO RELATED APPLICATION

This application is a continuation of U.S. application Ser. No. 18/044,706, filed on Mar. 9, 2023, which is the National Stage entry of International Application No. PCT/US2021/049895, filed on Sep. 10, 2021, which claims the benefit of and priority to U.S. Provisional Patent Application No. 63/077,330, filed Sep. 11, 2020, the disclosures of which are incorporated by reference herein in their entireties for all purposes.

### SEQUENCE LISTING

The instant application contains a sequence listing. The contents of the electronic sequence listing (CLS-023USC1_SL.xml; Size: 6,668 bytes; and Date of Creation: Apr. 24, 2024) is herein incorporated by reference in its entirety.

### BACKGROUND

Cancer immunotherapy regimens targeting immune evasion mechanisms including checkpoint blockade (e.g. PD-1/PD-L1 and CTLA-4 blocking antibodies) have been shown to be effective in treating in a variety of cancers, dramatically improving outcomes in some population's refractory to conventional therapies. However, incomplete clinical responses and the development of intrinsic or acquired resistance will continue to limit the patient populations who could benefit from checkpoint blockade.

Protein tyrosine phosphatase non-receptor type 2 (PTPN2), also known as T cell protein tyrosine phosphatase (TC-PTP), is an intracellular member of the class 1 subfamily of phospho-tyrosine specific phosphatases that control multiple cellular regulatory processes by removing phosphate groups from tyrosine substrates. PTPN2 is ubiquitously expressed, but expression is highest in hematopoietic and placental cells (Mosinger, B. Jr, et al., Proc Natl Acad Sci USA 89:499-503:1992). In humans, PTPN2 expression is controlled post-transcriptionally by the existence of two splice variants; a 45 kDa form that contains a nuclear localization signal at the C-terminus upstream of the splice junction, and a 48 kDa canonical form which has a C-terminal ER retention motif (Tillmann U, et al., Mol Cell Biol 14:3030-3040; 1994). The 45 kDa isoform can passively transfuse into the cytosol under certain cellular stress conditions. Both isoforms share an N-terminal phospho-tyrosine phosphatase catalytic domain. PTPN2 negatively regulates signaling of non-receptor tyrosine kinases (e.g. JAK1, JAK3), receptor tyrosine kinases (e.g. INSR, EGFR, CSFIR, PDGFR), transcription factors (e.g. STAT1, STAT3, STAT5a/b), and Src family kinases (e.g. Fyn, Lck). As a critical negative regulator of the JAK-STAT pathway, PTPN2 functions to directly regulate signaling through cytokine receptors, including IFNγ. The PTPN2 catalytic domain shares 74% sequence homology with PTPN1 (also called PTPIB), and shares similar enzymatic kinetics (Romsicki Y, et al., Arch Biochem Biophys 414:40-50:2003).

Data from a loss of function in vivo genetic screen using CRISPR/Cas9 genome editing in a mouse B16F10 transplantable tumor model show that deletion of Ptpn2 gene in tumor cells improved response to the immunotherapy regimen of a GM-CSF secreting vaccine (GVAX) plus PD-1 checkpoint blockade (Manguso R. T, et al., Nature 547:413-418:2017). Loss of Ptpn2 sensitized tumors to immunotherapy by enhancing IFNγ-mediated effects on antigen presentation and growth suppression. The same screen also revealed that genes known to be involved in immune evasion, including PD-L1 and CD47, were also depleted under immunotherapy selective pressure, while genes involved in the IFNγ signaling pathway, including IFNGR, JAK1, and STAT1, were enriched. These observations point to a putative role for therapeutic strategies that enhance IFNγ sensing and signaling in enhancing the efficacy of cancer immunotherapy regimens.

Protein tyrosine phosphatase non-receptor type 1 (PTPN1), also known as protein tyrosine phosphatase-1B (PTPIB), has been shown to play a key role in insulin and leptin signaling and is a primary mechanism for down-regulating both the insulin and leptin receptor signaling pathways (Kenner K. A, et al., J Biol Chem 271:19810-19816, 1996). Animals deficient in PTPN1 have improved glucose regulation and lipid profiles and are resistant to weight gain when treated with a high fat diet (Elchebly M, et al., Science 283:1544-1548, 1999). Thus, PTPN1 inhibitors are expected to be useful for the treatment of type 2 diabetes, obesity, and metabolic syndrome.

### SUMMARY

The present disclosure is directed, at least in part, to compounds, compositions, and methods for the inhibition of protein tyrosine phosphatase, e.g., protein tyrosine phosphatase non-receptor type 2 (PTPN2) and/or protein tyrosine phosphatase non-receptor type 1 ((PTPN1), also known as protein tyrosine phosphatase-1B (PTPIB)). In some embodiments, disclosed herein is an inhibitor of protein tyrosine phosphatase, e.g., PTPN2 and/or PTPN1, comprising a compound disclosed herein. In other embodiments, disclosed herein are methods of treating a disease or disorder, e.g., cancer, type-2 diabetes, obesity, a metabolic disease, or any other disease, disorder or ailment favorably responsive to PTPN2 or PTPN1 inhibitor treatment, comprising administering an effective amount of a compound disclosed herein.

For example, disclosed herein is a compound represented by Formula (I):

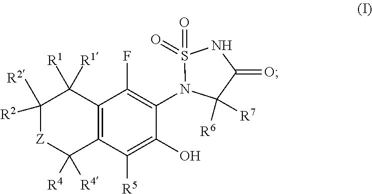

or a pharmaceutically acceptable salt thereof, wherein:
Z is selected from the group consisting of C(R³)(R^{3′}), a bond and N(R⁸);
R¹ is selected from the group consisting of hydrogen, deuterium, halogen, -hydroxyl, C₁₋₆alkyl, C₃₋₆cycloalkyl and —O—C₁₋₆alkyl;
   wherein C₁₋₆alkyl, C₃₋₆cycloalkyl and —O—C₁₋₆alkyl may optionally be substituted on one or more available carbons by one, two, three or more substituents each independently selected from R^{g};
R^{1′} is selected from the group consisting of hydrogen and deuterium;
R² is selected from the group consisting of hydrogen, hydroxyl, —C₁₋₆alkyl, —C₂₋₆alkenyl, —O—C₁₋₆alkyl, —NH₂, —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{c})₃, —C₁₋₆alkylene-N(R^{a})—C₁₋₆alkyl, —C₁₋₆alkylene-N(R^{a})—C₁₋₆alkylene-C₃₋₆cycloalkyl, —C₁₋₆alkylene-N(R^{a})(R^{b}), —C₁₋₆alkylene-N(R^{a})—C(O)—O—C₁₋₆alkyl, —N(R^{a})—(C═N(R^{b}))—C₁₋₆alkyl, —S(O)_{w}—C₁₋₆alkyl, —C(O)—N(R^{a})—C₁₋₆alkyl, —N(R^{a})—C(O)—C₁₋₆alkyl, —N(R^{a})—S(O)_{w}—C₁₋₆alkyl, —O—C(O)—N(R^{a})—C₁₋₆alkyl, —O—C(O)—N(R^{a})-phenyl, —N(R^{a})—C(O)—O—C₁₋₆alkyl, C₃₋₆cycloalkyl, —C₁₋₆alkylene-C₃₋₆cycloalkyl, —O—C₁₋₆alkylene-C₃₋₆cycloalkyl, 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl, —N(R^{a})—C₁₋₆alkylene-5-6 membered heteroaryl and —N(R^{a})—C₁₋₆alkylene-phenyl;
   wherein —C₁₋₆alkyl, —C₂₋₆alkenyl, —O—C₁₋₆alkyl, —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{c})₃, —C₁₋₆alkylene-N(R^{a})—C₁₋₆alkyl, —C₁₋₆alkylene-N(R^{a})—C₁₋₆alkylene-C₃₋₆cycloalkyl, —N(R^{a})—(C═N(R^{b}))—C₁₋₆alkyl, —S(O)_{w}—C₁₋₆alkyl, —C(O)—N(R^{a})—C₁₋₆alkyl, —N(R^{a})—C(O)—C₁₋₆alkyl, —O—C(O)—N(R³)—C₁₋₆alkyl, —O—C(O)—N(R^{a})-phenyl, —N(R^{a})—C(O)—O—C₁₋₆alkyl, C₃₋₆cycloalkyl, —C₁₋₆alkylene-C₃₋₆cycloalkyl, —O—C₁₋₆alkylene-C₃₋₆cycloalkyl, 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl, —N(R^{a})—C₁₋₆alkylene-5-6 membered heteroaryl and —N(R^{a})—C₁₋₆alkylene-phenyl may optionally be substituted on one or more available carbons by one, two, three or more substituents each independently selected from R^{g};
   wherein if 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl or —N(R^{a})—C₁₋₆alkylene-5-6 membered heteroaryl contains a substitutable ring nitrogen atom, that ring nitrogen atom may optionally be substituted by R^{h}, and
   wherein if Z is C(H)(R³), then R² is not —CH₂—CH₃;
R^{2′} is selected from the group consisting of hydrogen, deuterium, hydroxyl, —NR^{a}R^{b} and —N(R^{a})—N(R^{b})—C(O)-phenyl;
R³ is selected from the group consisting of hydrogen, deuterium, -hydroxyl, —C₁₋₆alkyl, —O—C₁₋₆alkyl, —O—C₁₋₆alkylene-C₃₋₆cycloalkyl, —N(R^{a})—C₁₋₆alkyl, —N(R^{a})—C₁₋₆alkylene-C₃₋₆cycloalkyl, —S(O)_{w}—C₁₋₆alkyl, —C(O)—N(R^{a})—C₁₋₆alkyl, —N(R^{a})—C(O)—C₁₋₆alkyl and —C₁₋₆alkylene-4-6 membered heterocyclyl;
   wherein —C₁₋₆alkyl, —O—C₁₋₆alkyl, —O—C₁₋₆alkylene-C₃₋₆cycloalkyl, —N(R^{a})—C₁₋₆alkyl, —N(R^{a})—C₁₋₆alkylene-C₃₋₆cycloalkyl, —S(O)_{w}—C₁₋₆alkyl, —C(O)—N(R^{a})—C₁₋₆alkyl, —N(R^{a})—C(O)—C₁₋₆alkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R^{3′} is selected from the group consisting of hydrogen and deuterium;
R⁴ is selected from the group consisting of hydrogen, halogen, C₁₋₆alkyl, C₃₋₆cycloalkyl and —C₁₋₆alkylene-4-6 membered heterocyclyl;
   wherein C₁₋₆alkyl, C₃₋₆cycloalkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R^{4′} is selected from the group consisting of hydrogen and deuterium;
R⁵ is selected from the group consisting of hydrogen, deuterium, halogen, C₁₋₆alkyl, C₃₋₆cycloalkyl and —C₁₋₆alkylene-4-6 membered heterocyclyl;
   wherein C₁₋₆alkyl, C₃₋₆cycloalkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R⁶ is selected from the group consisting of hydrogen and deuterium;
R⁷ is selected from the group consisting of hydrogen and deuterium;
R⁸ is selected from the group consisting of hydrogen and C₁₋₆alkyl;
R^{g} is independently selected for each occurrence from the group consisting of hydrogen, deuterium, halogen, hydroxyl, cyano, nitro, oxo, R^{a}R^{b}N—, R^{a}R^{b}N—C(O)—, R^{a}R^{b}N—SO_{w}—, R^{a}R^{b}N—C(O)—N(R^{a})—, C₁₋₆alkyl, C₂₋₆alkenyl, C₂₋₆alkynyl, C₃₋₆cycloalkyl, C₃₋₆cycloalkyl-C₁₋₆alkylene-, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{a})—, C₁₋₆alkyl-N(R^{a})—C(O)—, C₁₋₆alkyl-C(O)—N(R^{a}), C₁₋₆alkyl-N(R^{a})—C(O)—N(R^{a})—, C₁₋₆alkyl-N(R^{a})—SO_{w}—, C₃₋₆cycloalkyl-N(R^{a})—SO_{w}—, C₁₋₆alkyl-SO_{w}—N(R^{a})—, C₃₋₆cycloalkyl-SO_{w}—N(R^{a})—, C₁₋₆alkoxy-C(O)—N(R^{a})—, C₁₋₆alkyl-C(O)—N(R^{a})—C₁₋₆alkyl-, C₁₋₆alkyl-N(R^{a})—C(O)—C₁₋₆alkyl- and C₁₋₆alkoxy-C₁₋₆alkyl-;
   wherein C₁₋₆alkyl, C₂₋₆alkenyl, C₂₋₆alkynyl, C₃₋₆cycloalkyl, —C₁₋₆alkylene-C₃₋₆cycloalkyl, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{a})—, C₁₋₆alkyl-N(R^{a})—C(O)—, C₁₋₆alkyl-C(O)—N(R^{a}), C₁₋₆alkyl-N(R^{a})—C(O)—N(R^{a})—, C₁₋₆alkyl-N(R^{a})—SO_{w}—, C₃₋₆cycloalkyl-N(R^{a})—SO_{w}—, C₁₋₆alkyl-SO_{w}—N(R^{a})—, C₃₋₆cycloalkyl-SO_{w}—N(R^{a})—, C₁₋₆alkoxy-C(O)—N(R³)—, C₁₋₆alkyl-C(O)—N(R^{a})—C₁₋₆alkyl-, C₁₋₆alkyl-N(R^{a})—C(O)—C₁₋₆alkyl- and C₁₋₆alkoxy-C₁₋₆alkyl- may optionally be substituted by one, two three or more substituents each independently selected from R^{P};
R^{h} is independently selected for each occurrence from the group consisting of C₁₋₆alkyl, C₃₋₆alkenyl, C₃₋₆alkynyl, C₃₋₆cycloalkyl, —C₁₋₆alkylene-C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{a}R^{b}N—C(O)— and R^{a}R^{b}N—SO₂—;
   wherein C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{a}R^{b}N—C(O)— and R^{a}R^{b}N—SO₂— may optionally be substituted by one, two three or more substituents each independently selected from R^{P};
R^{P} is independently selected for each occurrence from the group consisting of halogen, deuterium, hydroxyl, cyano, C₁₋₆alkoxy, C₃₋₆cycloalkyl, R^{a}R^{b}N—, R^{a}R^{b}N-carbonyl-, R^{a}R^{b}N—SO₂—, and R^{a}R^{b}N-carbonyl-N(R^{a})—;
R^{a} and R^{b} are independently selected, for each occurrence, from the group consisting of hydrogen and C₁₋₆alkyl; wherein C₁₋₆alkyl may optionally be substituted by one or more substituents each independently selected from the group consisting of halogen, cyano, oxo and hydroxyl;
or R^{a} and R^{b} together with the nitrogen to which they are attached form a 4-6 membered heterocyclyl, wherein 4-6 membered heterocyclyl may optionally be substituted by one or more substituents each independently selected from the group consisting of halogen, cyano, oxo and hydroxyl;
R^{c} is independently selected, for each occurrence, from the group consisting of hydroxyl, C₁₋₄alkyl and phenyl; and
w is 0, 1 or 2.

Also disclosed herein is a compound represented by Formula (II):

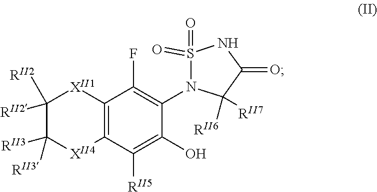

or a pharmaceutically acceptable salt thereof, wherein:
X^{II1} is selected from the group consisting of O and C(R^{II1})(R^{II1′});
X^{II4} is selected from the group consisting of O and C(R^{II4})(R^{II4′});
   wherein at least one of X^{II1} and X^{II4} is O;
R^{II1} and R^{II1′} are each independently selected from the group consisting of hydrogen, halogen, -hydroxyl, C₁₋₆alkyl, C₂₋₆alkenyl, C₂₋₆alkynyl and C₃₋₆cycloalkyl;
   wherein C₁₋₆alkyl, C₂₋₆alkenyl, C₂₋₆alkynyl and C₃₋₆cycloalkyl may optionally be substituted on one or more available carbons by one, two, three or more substituents each independently selected from R^{IIg};
R^{II2} is selected from the group consisting of hydrogen, C₁₋₆alkyl, C₂₋₆alkenyl, C₂₋₆alkynyl, —O—C₁₋₆alkyl, —NH(R^{IIa}), —N(R^{IIa})—C₁₋₆alkyl, —N(R^{IIa})—C₁₋₆alkylene-C₃₋₆cycloalkyl, —S(O)₂—C₁₋₆alkyl, —C(O)—N(R^{IIa})—C₁₋₆alkyl, —N(R^{IIa})—C(O)—C₁₋₆alkyl, —O—C(O)—N(R^{IIa})—C₁₋₆alkyl, —N(R^{IIa})—C(O)—O—C₁₋₆alkyl, C₃₋₆cycloalkyl, phenyl, 5-6 membered heteroaryl, 4-6 membered heterocyclyl, —C₁₋₆alkylene-C₃₋₆cycloalkyl, —C₁₋₆alkylene-phenyl, —C₁₋₆alkylene-5-6 membered heteroaryl, —C₁₋₆alkylene-4-6 membered heterocyclyl, —O—C₁₋₆alkylene-C₃₋₆cycloalkyl, —N(R^{IIa})-4-6 membered heterocyclyl, —O—C₁₋₆alkylene-4-6 membered heterocyclyl, —N(R^{IIa})—C₁₋₆alkylene-4-6 membered heterocyclyl, —N(R^{IIa})—C₁₋₆alkylene-5-6 membered heteroaryl and —N(R^{IIa})—C₁₋₆alkylene-phenyl;
   wherein C₁₋₆alkyl, C₂₋₆alkenyl, C₂₋₆alkynyl, —O—C₁₋₆alkyl, —N(R^{IIa})—C₁₋₆alkyl, —N(R^{IIa})—C₁₋₆alkylene-C₃₋₆cycloalkyl, —S(O)₂—C₁₋₆alkyl, —C(O)—N(R^{IIa})—C₁₋₆alkyl, —N(R^{IIa})—C(O)—C₁₋₆alkyl, —O—C(O)—N(R^{IIa})—C₁₋₆alkyl, —N(R^{IIa})—C(O)—O—C₁₋₆alkyl, C₃₋₆cycloalkyl, phenyl, 5-6 membered heteroaryl, 4-6 membered heterocyclyl, —C₁₋₆alkylene-C₃₋₆cycloalkyl, —C₁₋₆alkylene-phenyl, —C₁₋₆alkylene-5-6 membered heteroaryl, —C₁₋₆alkylene-4-6 membered heterocyclyl, —O—C₁₋₆alkylene-C₃₋₆cycloalkyl, —N(R^{IIa})-4-6 membered heterocyclyl, —O—C₁₋₆alkylene-4-6 membered heterocyclyl, —N(R^{IIa})—C₁₋₆alkylene-4-6 membered heterocyclyl, —N(R^{IIa})—C₁₋₆alkylene-5-6 membered heteroaryl and —N(R^{IIa})—C₁₋₆alkylene-phenyl may optionally be substituted on one or more available carbons by one, two, three or more substituents each independently selected from R^{IIg};
   wherein if 5-6 membered heteroaryl, 4-6 membered heterocyclyl, —C₁₋₆alkylene-5-6 membered heteroaryl, —C₁₋₆alkylene-4-6 membered heterocyclyl, —N(R^{IIa})-4-6 membered heterocyclyl, —O—C₁₋₆alkylene-4-6 membered heterocyclyl, —N(R^{IIa})—C₁₋₆alkylene-4-6 membered heterocyclyl, or —N(R^{IIa})—C₁₋₆alkylene-5-6 membered heteroaryl contains a substitutable ring nitrogen atom, that ring nitrogen atom may optionally be substituted by R^{IIh}; and
   wherein if R^{II2} is —O—C₁₋₆alkyl, —N(R^{IIa})—C₁₋₆alkyl, —N(R^{IIa})—C₁₋₆alkylene-C₃₋₆cycloalkyl, —S(O)₂—C₁₋₆alkyl, —N(R^{IIa})—C(O)—C₁₋₆alkyl, —O—C(O)—N(R^{IIa})—C₁₋₆alkyl, —N(R^{IIa})—C(O)—O—C₁₋₆alkyl, —O—C₁₋₆alkylene-C₃₋₆cycloalkyl, —N(R^{IIa})-4-6 membered heterocyclyl, —O—C₁₋₆alkylene-4-6 membered heterocyclyl, —N(R^{IIa})—C₁₋₆alkylene-4-6 membered heterocyclyl, —N(R^{IIa})—C₁₋₆alkylene-5-6 membered heteroaryl or —N(R^{IIa})—C₁₋₆alkylene-phenyl; then X^{II1} is C(R^{II1})(R^{II1′}) and X^{II4} is O;
R^{II2′} is selected from the group consisting of hydrogen, C₁₋₆alkyl, C₂₋₆alkenyl, C₂₋₆alkynyl, C₃₋₆cycloalkyl, phenyl, 5-6 membered heteroaryl, 4-6 membered heterocyclyl, —C₁₋₆alkylene-C₃₋₆cycloalkyl, —C₁₋₆alkylene-phenyl, —C₁₋₆alkylene-5-6 membered heteroaryl and —C₁₋₆alkylene-4-6 membered heterocyclyl;
   wherein C₁₋₆alkyl, C₂₋₆alkenyl, C₂₋₆alkynyl. C₃₋₆cycloalkyl, phenyl, 5-6 membered heteroaryl, 4-6 membered heterocyclyl, —C₁₋₆alkylene-C₃₋₆cycloalkyl, —C₁₋₆alkylene-phenyl, —C₁₋₆alkylene-5-6 membered heteroaryl and —C₁₋₆alkylene-4-6 membered heterocyclyl may optionally be substituted on one or more available carbons by one, two, three or more substituents each independently selected from R^{IIg}; and
   wherein if 5-6 membered heteroaryl, 4-6 membered heterocyclyl, —C₁₋₆alkylene-5-6 membered heteroaryl or —C₁₋₆alkylene-4-6 membered heterocyclyl contains a substitutable ring nitrogen atom, that ring nitrogen atom may optionally be substituted by R^{IIh};
R^{II3} and R^{II3′} are each independently selected from the group consisting of hydrogen, C₁₋₆alkyl, C₂₋₆alkenyl, C₂₋₆alkynyl and C₃₋₆cycloalkyl;
   wherein C₁₋₆alkyl, C₂₋₆alkenyl, C₂₋₆alkynyl and C₃₋₆cycloalkyl may optionally be substituted on one or more available carbons by one, two, three or more substituents each independently selected from R^{IIg};
R^{II4} and R^{II4′} are each independently selected from the group consisting of hydrogen, halogen, C₁₋₆alkyl, C₂₋₆alkenyl, C₂₋₆alkynyl and C₃₋₆cycloalkyl;
   wherein C₁₋₆alkyl, C₂₋₆alkenyl, C₂₋₆alkynyl and C₃₋₆cycloalkyl may optionally be substituted on one or more available carbons by one, two, three or more substituents each independently selected from R^{IIg};
R^{II5} is selected from the group consisting of hydrogen, deuterium, halogen, C₁₋₆alkyl and C₃₋₆cycloalkyl;
   wherein C₁₋₆alkyl and C₃₋₆cycloalkyl may optionally be substituted on one or more available carbons by one, two, three or more substituents each independently selected from R^{IIg};
R^{II6} is selected from the group consisting of hydrogen and deuterium;
R^{II7} is selected from the group consisting of hydrogen and deuterium;
R^{IIg} is independently selected for each occurrence from the group consisting of hydrogen, halogen, hydroxyl, cyano, nitro, oxo, phenyl, 5-6 membered heteroaryl, R^{IIa}R^{IIb}N—, R^{IIa}R^{IIb}N—C(O)—, R^{IIa IIb}N—SO_{w}—, R^{IIa}R^{IIb}N—C(O)—N(R^{IIIa})—, C₁₋₆alkyl, C₂₋₆alkenyl, C₂₋₆alkynyl, C₃₋₆cycloalkyl, C₃₋₆cycloalkyl-C₁₋₆alkylene-, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{IIa})—, C₁₋₆alkyl-N(R^{IIa})—C(O)—, C₁₋₆alkyl-C(O)—N(R^{IIa})—, C₁₋₆alkyl-N(R^{IIa})—C(O)—N(R^{IIa})—, C₁₋₆alkyl-N(R^{IIa})—SO_{w}—, C₃₋₆cycloalkyl-N(R^{IIa})—SO_{w}—, C₁₋₆alkyl-SO_{w}—N(R^{IIa})—, C₃₋₆cycloalkyl-SO_{w}—N(R^{IIa})—, C₁₋₆alkoxy-C(O)—N(R^{IIa})—, C₁₋₆alkyl-C(O)—N(R^{IIa})—C₁₋₆alkyl-, C₁₋₆alkyl-N(R^{IIa})—C(O)—C₁₋₆alkyl- and C₁₋₆alkoxy-C₁₋₆alkyl-;
   wherein C₁₋₆alkyl, C₂₋₆alkenyl, C₂₋₆alkynyl, C₃₋₆cycloalkyl, —C₁₋₆alkylene-C₃₋₆cycloalkyl, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{IIa})—, C₁₋₆alkyl-N(R^{IIa})—C(O)—, C₁₋₆alkyl-C(O)—N(R^{IIa})—, C₁₋₆alkyl-N(R^{IIa})—C(O)—N(R^{IIa})—, C₁₋₆alkyl-N(R^{IIa})—SO_{w}—, C₃₋₆cycloalkyl-N(R^{IIa})—SO_{w}—, C₁₋₆alkyl-SO_{w}—N(R^{IIa})—, C₃₋₆cycloalkyl-SO_{w}—N(R^{IIa})—, C₁₋₆alkoxy-C(O)—N(R^{IIa})—, C₁₋₆alkyl-C(O)—N(R^{IIa})—C₁₋₆alkyl-, C₁₋₆alkyl-N(R^{IIa})—C(O)—C₁₋₆alkyl- and C₁₋₆alkoxy-C₁₋₆alkyl- may optionally be substituted by one, two three or more substituents each independently selected from R^{IIP};
R^{IIh} is independently selected for each occurrence from the group consisting of C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{IIa}R^{IIb}N—C(O)— and R^{IIa}R^{IIb}N—SO₂—;
   wherein C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{IIa}R^{IIb}N—C(O)— and R^{IIa}R^{IIb}N—SO₂— may optionally be substituted by one, two three or more substituents each independently selected from R^{IIP};
R^{IIP} is independently selected for each occurrence from the group consisting of halogen, hydroxyl, cyano, C₁₋₆alkoxy, C₃₋₆cycloalkyl, R^{IIa}R^{IIb}N—, R^{IIa}R^{IIb}N-carbonyl-, R^{IIa}R^{IIb}N—SO₂—, and R^{IIa}R^{IIb}N-carbonyl-N(R^{IIa})—;
R^{IIa} and R^{IIb} are independently selected, for each occurrence, from the group consisting of hydrogen and C₁₋₃alkyl; wherein C₁₋₃alkyl may optionally be substituted by one or more substituents each independently selected from the group consisting of halogen, cyano, oxo and hydroxyl;
or R^{IIa} and R^{IIb} together with the nitrogen to which they are attached form a 4-6 membered heterocyclyl, wherein 4-6 membered heterocyclyl may optionally be substituted by one or more substituents each independently selected from the group consisting of halogen, cyano, oxo and hydroxyl; and
w is 0, 1 or 2.

Further disclosed herein is a compound represented by Formula (III):

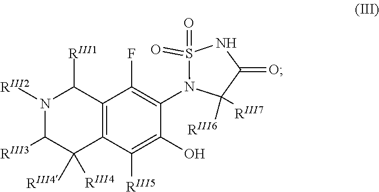

or a pharmaceutically acceptable salt thereof, wherein:
R^{III1} is selected from the group consisting of hydrogen, oxo, C₁₋₆alkyl, C₂₋₆alkenyl and C₂₋₆alkynyl;
R^{III2} is selected from the group consisting of hydrogen, C₁₋₆alkyl, C₂₋₆alkenyl, C₂₋₆alkynyl, C₃₋₆cycloalkyl, phenyl, 4-7 membered heterocyclyl, 5-6 membered heteroaryl, —C₁₋₆alkylene-C₃₋₈cycloalkyl, —C₁₋₆alkylene-phenyl, —C₁₋₆alkylene-4-7 membered heterocyclyl, —C₁₋₆alkylene-5-6 membered heteroaryl, —C(O)—C₁₋₆alkyl, —C(O)—O—C₁₋₆alkyl, —C(O)—C₁₋₆alkylene-C₃₋₈cycloalkyl, —C(O)—N(R^{IIIa})—C₁₋₆alkyl, —C(O)—N(R^{IIIa})—C₁₋₆alkylene-C₃₋₆cycloalkyl, —C(O)—N(R^{IIIa})—C₁₋₆alkylene-phenyl, —C(O)—N(R^{IIIa})—C₁₋₆alkylene-4-7 membered heterocyclyl, —C(O)—N(R^{IIIa})—C₁₋₆alkylene-5-6 membered heteroaryl, —C═N(R^{IIIa})—C₁₋₆alkyl, —C═N(R^{IIIa})—N(R^{IIIa})—C₁₋₆alkyl, —S(O)₂—N(R^{IIIa})—C₁₋₆alkyl, and —S(O)₂—C₁₋₆alkyl;
   wherein C₁₋₆alkyl, C₂₋₆alkenyl, C₂₋₆alkynyl, C₃₋₆cycloalkyl, phenyl, 4-7 membered heterocyclyl, 5-6 membered heteroaryl, —C₁₋₆alkylene-C₃₋₈cycloalkyl, —C₁₋₆alkylene-phenyl, —C₁₋₆alkylene-4-7 membered heterocyclyl, —C₁₋₆alkylene-5-6 membered heteroaryl, —C(O)—C₁₋₆alkyl, —C(O)—O—C₁₋₆alkyl, —C(O)—C₁₋₆alkylene-C₃₋₈cycloalkyl, —C(O)—N(R^{IIIa})—C₁₋₆alkyl, —C(O)—N(R^{IIIa})—C₁₋₆alkylene-C₃₋₆cycloalkyl, —C(O)—N(R^{IIIa})—C₁₋₆alkylene-phenyl, —C(O)—N(R^{IIIa})—C₁₋₆alkylene-4-7 membered heterocyclyl, —C(O)—N(R^{IIIa})—C₁₋₆alkylene-5-6 membered heteroaryl, —C═N(R^{IIIa})—C₁₋₆alkyl and —S(O)₂—C₁₋₆alkyl may optionally be substituted on one or more available carbons by one, two, three or more substituents each independently selected from R^{IIIg}; and
   wherein if 4-7 membered heterocyclyl, 5-6 membered heteroaryl, —C₁₋₆alkylene-4-7 membered heterocyclyl, —C₁₋₆alkylene-5-6 membered heteroaryl, —C(O)—N(R^{IIa})—C₁₋₆alkylene-4-7 membered heterocyclyl or —C(O)—N(R^{IIIa})—C₁₋₆alkylene-5-6 membered heteroaryl contains a substitutable ring nitrogen atom, that ring nitrogen atom may optionally be substituted by R^{IIIh};
R^{III3} is selected from the group consisting of hydrogen, C₁₋₆alkyl, C₂₋₆alkenyl and C₂₋₆alkynyl;
R^{III4} is selected from the group consisting of hydrogen, halogen, C₁₋₆alkyl, C₂₋₆alkenyl and C₂₋₆alkynyl;
R^{III4} is selected from the group consisting of hydrogen, halogen, C₁₋₆alkyl, C₂₋₆alkenyl and C₂₋₆alkynyl;
R^{III5} is selected from the group consisting of hydrogen, halogen and C₁₋₆alkyl;
R^{III6} is selected from the group consisting of hydrogen and deuterium;
R^{III7} is selected from the group consisting of hydrogen and deuterium;
R^{IIIg} is independently selected for each occurrence from the group consisting of hydrogen, halogen, hydroxyl, cyano, nitro, oxo, R^{IIIa}R^{IIIb}N—, R^{IIIa}R^{IIIb}N—C(O)—, R^{IIIa IIIb}N—SO_{w}—, R^{IIIa}R^{IIIb}N—C(O)—N(R^{IIIa})—, C₁₋₆alkyl, C₂₋₆alkenyl, C₂₋₆alkynyl, C₃₋₆cycloalkyl, C₃₋₆cycloalkyl-C₁₋₆alkylene-, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{IIIa})—, C₁₋₆alkyl-N(R^{IIIa})—C(O)—, C₁₋₆alkyl-C(O)—N(R^{IIIa}), C₁₋₆alkyl-N(R^{IIIa})—C(O)—N(R^{IIIa})—, C₁₋₆alkyl-N(R^{IIIa})—SO_{w}—, C₃₋₆cycloalkyl-N(R^{IIIa})—SO_{w}—, C₁₋₆alkyl-SO_{w}—N(R^{IIIa})—, C₃₋₆cycloalkyl-SO_{w}—N(R^{IIIa})—, C₁₋₆alkoxy-C(O)—N(R^{IIIa})—, C₁₋₆alkyl-C(O)—N(R^{IIIa})—C₁₋₆alkyl-, C₁₋₆alkyl-N(R^{IIIa})—C(O)—C₁₋₆alkyl-, C₁₋₆alkoxy-C₁₋₆alkyl- and 5-6 membered heteroaryl;
   wherein C₁₋₆alkyl, C₂₋₆alkenyl, C₂₋₆alkynyl, C₃₋₆cycloalkyl, —C₁₋₆alkylene-C₃₋₆cycloalkyl, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{IIIa})—, C₁₋₆alkyl-N(R^{IIIa})—C(O)—, C₁₋₆alkyl-C(O)—N(R^{IIIa}), C₁₋₆alkyl-N(R^{IIIa})—C(O)—N(R^{IIIa})—, C₁₋₆alkyl-N(R^{IIIa})—SO_{w}—, C₃₋₆cycloalkyl-N(R^{IIIa})—SO_{w}—, C₁₋₆alkyl-SO_{w}—N(R^{IIIa})—, C₃₋₆cycloalkyl-SO_{w}—N(R^{IIIa})—, C₁₋₆alkoxy-C(O)—N(R^{IIIa})—, C₁₋₆alkyl-C(O)—N(R^{IIIa})—C₁₋₆alkyl-, C₁₋₆alkyl-N(R^{IIa})—C(O)—C₁₋₆alkyl-, C₁₋₆alkoxy-C₁₋₆alkyl- and 5-6 membered heteroaryl may optionally be substituted by one, two three or more substituents each independently selected from R^{IIIP};
R^{IIIh} is independently selected for each occurrence from the group consisting of C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{IIIa}R^{IIIb}N—C(O)—, R^{IIIa}R^{IIIb}N—SO₂— and —C₁₋₆alkylene-5-6 membered heteroaryl;
   wherein C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{IIIa}R^{IIIb}N—C(O)—, R^{IIIa}R^{IIIb}N—SO₂— and —C₁₋₆alkylene-5-6 membered heteroaryl may optionally be substituted by one, two three or more substituents each independently selected from R^{IIIP};
R^{IIIP} is independently selected for each occurrence from the group consisting of halogen, hydroxyl, cyano, C₁₋₆alkoxy, C₃₋₆cycloalkyl, R^{IIIa}R^{IIIb}N—, R^{IIIa}R^{IIIb}N-carbonyl-, R^{IIIa}R^{IIIb}N—SO₂—, and R^{IIIa}R^{IIIb}N-carbonyl-N(R^{IIIa})—;
R^{IIIa} and R^{IIIb} are independently selected, for each occurrence, from the group consisting of hydrogen and C₁₋₃alkyl;
   wherein C₁₋₃alkyl may optionally be substituted by one or more substituents each independently selected from the group consisting of halogen, cyano, oxo and hydroxyl:
or R^{IIIa} and R^{IIIb} together with the nitrogen to which they are attached form a 4-6 membered heterocyclyl;
   wherein 4-6 membered heterocyclyl may optionally be substituted by one or more substituents each independently selected from the group consisting of halogen, cyano, oxo and hydroxyl; and
w is 0, 1 or 2.

Further disclosed herein is a compound selected from the group consisting of:
5-[(3S)-3-amino-5-fluoro-7-hydroxy-3,4-dihydro-2H-1-benzopyran-6-yl]-1λ⁶,2,5-thiadiazolidine-1,1,3-trione;
5-{(7R)-1-fluoro-3-hydroxy-7-[(3-methylbutyl)amino]-5,6,7,8-tetrahydronaphthalen-2-yl}(4,4-²H₂)-1λ⁶,2,5-thiadiazolidine-1,1,3-trione;
8-fluoro-6-hydroxy-N-(2-methylpropyl)-7-(1,1,4-trioxo-1λ⁶,2,5-thiadiazolidin-2-yl)-3,4-dihydroisoquinoline-2(1H)-sulfonamide:
8-fluoro-6-hydroxy-N-(2-methylpropyl)-7-(1,1,4-trioxo-1λ⁶,2,5-thiadiazolidin-2-yl)-3,4-dihydroisoquinoline-2(1H)-carboximidamide;
5-(1-fluoro-3-hydroxy-7-{[2-(oxetan-3-yl)ethyl]amino}-5,6,7,8-tetrahydronaphthalen-2-yl)-1λ⁶,2,5-thiadiazolidine-1,1,3-trione;
5-{(7R)-1,4-difluoro-3-hydroxy-7-[(3-methylbutyl)amino]-5,6,7,8-tetrahydronaphthalen-2-yl}-1λ⁶,2,5-thiadiazolidine-1,1,3-trione;
N-[8-fluoro-6-hydroxy-7-(1,1,4-trioxo-1λ⁶,2,5-thiadiazolidin-2-yl)-1,2,3,4-tetrahydronaphthalen-2-yl]-3-methylbutane-1-sulfonamide;
5-(1-fluoro-3-hydroxy-7-{[(2-methylpropyl)amino]methyl}-5,6,7,8-tetrahydronaphthalen-2-yl)-1λ⁶,2,5-thiadiazolidine-1,1,3-trione;
5-{1-fluoro-7-[(2-fluoro-3-methylbutyl)amino]-3-hydroxy-5,6,7,8-tetrahydronaphthalen-2-yl}-1λ⁶,2,5-thiadiazolidine-1,1,3-trione;
5-(1-fluoro-3,7-dihydroxy-5,6,7,8-tetrahydronaphthalen-2-yl)-1λ⁶,2,5-thiadiazolidine-1,1,3-trione;
5-{7-[(²H₉)butylamino]-1-fluoro-3-hydroxy-5,6,7,8-tetrahydronaphthalen-2-yl}-1λ⁶,2,5-thiadiazolidine-1,1,3-trione;
5-[7-(aminomethyl)-1-fluoro-3,7-dihydroxy-5,6,7,8-tetrahydronaphthalen-2-yl]-1λ⁶,2,5-thiadiazolidine-1,1,3-trione;
5-[(7R)-1-fluoro-3-hydroxy-7-({2-[1-(hydroxymethyl)cyclobutyl]ethyl}amino)-5,6,7,8-tetrahydronaphthalen-2-yl]-1λ⁶,2,5-thiadiazolidine-1,1,3-trione;
5-(1-fluoro-3,7-dihydroxy-7-{[(2-methylpropyl)amino]methyl}-5,6,7,8-tetrahydronaphthalen-2-yl)-1λ⁶,2,5-thiadiazolidine-1,1,3-trione;
5-{1-fluoro-3-hydroxy-7-[(3-methylbutyl)amino](6,6,7,8,8-²H₅)-5,6,7,8-tetrahydronaphthalen-2-yl}-1λ⁶,2,5-thiadiazolidine-1,1,3-trione;
tert-butyl [(2R)-8-fluoro-6-hydroxy-7-(1,1,4-trioxo-1λ⁶,2,5-thiadiazolidin-2-yl)-1,2,3,4-tetrahydronaphthalen-2-yl]carbamate;
5-[(7R)-1-fluoro-3-hydroxy-7-{[(thiophen-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(thiophen-2-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(3-methyloxetan-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(1-methyl-1H-pyrrol-2-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(1-methyl-1H-pyrrol-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(pyridin-3-yl)methyl]amino}-5,6,7,8-tetrahydronaphthalen-2-yl]-1λ⁶,2,5-thiadiazolidine-1,1,3-trione;
5-{(7R)-1-fluoro-3-hydroxy-7-[(3,3,3-trifluoro-2-methylpropyl)amino]-5,6,7,8-tetrahydronaphthalen-2-yl}-1λ⁶,2,5-thiadiazolidine-1,1,3-trione;
5-[(7R)-1-fluoro-3-hydroxy-7-{[(pyridazin-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(oxan-2-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(5-methyl-1,2-oxazol-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(oxan-3-yl)methyl]amino}-5,6,7,8-tetrahydronaphthalen-2-yl]-1λ⁶,2,5-thiadiazolidine-1,1,3-trione;
2-({[(2R)-8-fluoro-6-hydroxy-7-(1,1,4-trioxo-1λ⁶,2,5-thiadiazolidin-2-yl)-1,2,3,4-tetrahydronaphthalen-2-yl]amino}methyl)cyclopropane-1-carbonitrile;
5-{(7R)-7-[(3-ethoxypropyl)amino]-1-fluoro-3-hydroxy-5,6,7,8-tetrahydronaphthalen-2-yl}-1λ⁶,2,5-thiadiazolidine-1,1,3-trione;
5-[(7R)-7-({[1-(difluoromethyl)cyclopropyl]methyl}amino)-1-fluoro-3-hydroxy-5,6,7,8-tetrahydronaphthalen-2-yl]-1λ⁶,2,5-thiadiazolidine-1,1,3-trione;
5-[(7R)-1-fluoro-3-hydroxy-7-{[2-(oxolan-3-yl)ethyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(1-methyl-1H-imidazol-5-yl)methyl]amino}-5,6,7,8-tetrahydronaphthalen-2-yl]-1λ⁶,2,5-thiadiazolidine-1,1,3-trione;
5-[(7R)-7-{[2,2-dimethyl-3-(pyrrolidin-1-yl)propyl]amino}-1-fluoro-3-hydroxy-5,6,7,8-tetrahydronaphthalen-2-yl]-1λ⁶,2,5-thiadiazolidine-1,1,3-trione;
5-[(7R)-1-fluoro-3-hydroxy-7-({[5-(hydroxymethyl)furan-2-yl]methyl}amino)-5,6,7,8-tetrahydronaphthalen-2-yl]-1λ⁶,2,5-thiadiazolidine-1,1,3-trione;
5-{(7R)-1-fluoro-3-hydroxy-7-[(4-methoxybutyl)amino]-5,6,7,8-tetrahydronaphthalen-2-yl}-1λ⁶,2,5-thiadiazolidine-1,1,3-trione;
5-[(7R)-1-fluoro-3-hydroxy-7-{[(oxolan-3-yl)methyl]amino}-5,6,7,8-tetrahydronaphthalen-2-yl]-1λ⁶,2,5-thiadiazolidine-1,1,3-trione;
5-[(7R)-7-{[(2,2-difluorocyclopropyl)methyl]amino}-1-fluoro-3-hydroxy-5,6,7,8-tetrahydronaphthalen-2-yl]-1λ⁶,2,5-thiadiazolidine-1,1,3-trione;
5-{(7R)-1-fluoro-3-hydroxy-7-[(3-methoxypropyl)amino]-5,6,7,8-tetrahydronaphthalen-2-yl}-1λ⁶,2,5-thiadiazolidine-1,1,3-trione;
5-[(7R)-1-fluoro-3-hydroxy-7-{[(1,3-oxazol-5-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[2-(oxan-4-yl)ethyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(oxetan-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(1,3-thiazol-2-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(pyridazin-4-yl)methyl]amino}-5,6,7,8-tetrahydronaphthalen-2-yl]-1λ⁶,2,5-thiadiazolidine-1,1,3-trione;
5-{(7R)-1-fluoro-3-hydroxy-7-[(3-hydroxybutyl)amino]-5,6,7,8-tetrahydronaphthalen-2-yl}-1λ⁶,2,5-thiadiazolidine-1,1,3-trione;
5-[(7S)-1-fluoro-3-hydroxy-7-[(3-methylbutyl)amino](6,6,7,8,8-²H₅)-5,6,7,8-tetrahydronaphthalen-2-yl]-1λ⁶,2,5-thiadiazolidine-1,1,3-trione;
5-[(7R)-1-fluoro-3-hydroxy-7-[(3-methylbutyl)amino](6,6,7,8,8-²H₅)-5,6,7,8-tetrahydronaphthalen-2-yl]-1λ⁶,2,5-thiadiazolidine-1,1,3-trione;
5-{(3S)-5-fluoro-7-hydroxy-3-[(3-methylbutyl)amino]-3,4-dihydro-2H-1-benzopyran-6-yl}-1λ⁶,2,5-thiadiazolidine-1,1,3-trione;
5-{(3S)-3-[(4,4-difluorobutyl)amino]-5-fluoro-7-hydroxy-3,4-dihydro-2H-1-benzopyran-6-yl}-1λ⁶,2,5-thiadiazolidine-1,1,3-trione;
5-{(7R)-7-[(5-amino-3,3-dimethylpentyl)amino]-1-fluoro-3-hydroxy-5,6,7,8-tetrahydronaphthalen-2-yl}-1λ⁶,2,5-thiadiazolidine-1,1,3-trione;
5-[(3S)-5-fluoro-7-hydroxy-3-{[2-(oxan-4-yl)ethyl]amino}-3,4-dihydro-2H-1-benzopyran-6-yl]-1λ⁶,2,5-thiadiazolidine-1,1,3-trione;
5-[(7R)-7-({2-[1-(aminomethyl)cyclobutyl]ethyl}amino)-1-fluoro-3-hydroxy-5,6,7,8-tetrahydronaphthalen-2-yl]-1λ⁶,2,5-thiadiazolidine-1,1,3-trione;
5-[(7R)-7-({2-[1-(2-aminoethyl)cyclobutyl]ethyl}amino)-1-fluoro-3-hydroxy-5,6,7,8-tetrahydronaphthalen-2-yl]-1λ⁶,2,5-thiadiazolidine-1,1,3-trione;
5-[(3S)-5-fluoro-7-hydroxy-3-{[2-(2,6,6-trimethylcyclohex-1-en-1-yl)ethyl]amino}-3,4-dihydro-2H-1-benzopyran-6-yl]-1λ⁶,2,5-thiadiazolidine-1,1,3-trione;
5-[(3S)-3-{[3-(2,2-difluoroethoxy)propyl]amino}-5-fluoro-7-hydroxy-3,4-dihydro-2H-1-benzopyran-6-yl]-1λ⁶,2,5-thiadiazolidine-1,1,3-trione;
5-[(3S)-5-fluoro-7-hydroxy-3-({[4-(trifluoromethyl)cyclohexyl]methyl}amino)-3,4-dihydro-2H-1-benzopyran-6-yl]-1λ⁶,2,5-thiadiazolidine-1,1,3-trione;
5-[(3S)-5-fluoro-3-({[1-(fluoromethyl)cyclopropyl]methyl}amino)-7-hydroxy-3,4-dihydro-2H-1-benzopyran-6-yl]-1λ⁶,2,5-thiadiazolidine-1,1,3-trione;
5-[(3S)-5-fluoro-7-hydroxy-3-{[2-(oxolan-3-yl)ethyl]amino}-3,4-dihydro-2H-1-benzopyran-6-yl]-1λ⁶,2,5-thiadiazolidine-1,1,3-trione;
5-[(3S)-3-({[(1RS,5SR)-bicyclo[3.1.0]hexan-6-yl]methyl}amino)-5-fluoro-7-hydroxy-3,4-dihydro-2H-1-benzopyran-6-yl]-1λ⁶,2,5-thiadiazolidine-1,1,3-trione;
tert-butyl 4-({[(3S)-5-fluoro-7-hydroxy-6-(1,1,4-trioxo-1λ⁶,2,5-thiadiazolidin-2-yl)-3,4-dihydro-2H-1-benzopyran-3-yl]amino}methyl) piperidine-1-carboxylate;
5-[(3S)-5-fluoro-7-hydroxy-3-{[(3-phenylcyclobutyl)methyl]amino}-3,4-dihydro-2H-1-benzopyran-6-yl]-1λ⁶,2,5-thiadiazolidine-1,1,3-trione;
5-{(3S)-5-fluoro-7-hydroxy-3-[(3-phenylpropyl)amino]-3,4-dihydro-2H-1-benzopyran-6-yl}-1λ⁶,2,5-thiadiazolidine-1,1,3-trione;
5-[8-fluoro-6-hydroxy-2-(4-methylpentyl)-1-oxo-1,2,3,4-tetrahydroisoquinolin-7-yl]-1λ⁶,2,5-thiadiazolidine-1,1,3-trione;
5-(8-fluoro-6-hydroxy-1-oxo-1,2,3,4-tetrahydroisoquinolin-7-yl)-1λ⁶,2,5-thiadiazolidine-1,1,3-trione;
5-[7-(aminomethyl)-1-fluoro-3-hydroxy-5,6,7,8-tetrahydronaphthalen-2-yl]-1λ⁶,2,5-thiadiazolidine-1,1,3-trione;
5-(1-fluoro-3-hydroxy-7-{[(3-methylbutyl)amino]methyl}-5,6,7,8-tetrahydronaphthalen-2-yl)-1λ⁶,2,5-thiadiazolidine-1,1,3-trione;
tert-butyl {[8-fluoro-6-hydroxy-7-(1,1,4-trioxo-1λ⁶,2,5-thiadiazolidin-2-yl)-1,2,3,4-tetrahydronaphthalen-2-yl]methyl}carbamate;
tert-butyl [(2R)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate;
5-{(6R,7S)-1-fluoro-3,6-dihydroxy-7-[(3-methylbutyl)amino]-5,6,7,8-tetrahydronaphthalen-2-yl}-1λ⁶,2,5-thiadiazolidine-1,1,3-trione;
5-(7-{[(3-cyclopropylpropyl)amino]methyl}-1-fluoro-3-hydroxy-5,6,7,8-tetrahydronaphthalen-2-yl)-1λ⁶,2,5-thiadiazolidine-1,1,3-trione;
tert-butyl [(2R,4R)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate;
5-{7-[(butylamino)methyl]-1-fluoro-3-hydroxy-5,6,7,8-tetrahydronaphthalen-2-yl}-1λ⁶,2,5-thiadiazolidine-1,1,3-trione;
5-[(5R,7R)-7-amino-1-fluoro-3-hydroxy-5-methyl-5,6,7,8-tetrahydronaphthalen-2-yl]-1λ⁶,2,5-thiadiazolidine-1,1,3-trione;
5-[(5S,7R)-7-amino-1-fluoro-3-hydroxy-5-methyl-5,6,7,8-tetrahydronaphthalen-2-yl]-1λ⁶,2,5-thiadiazolidine-1,1,3-trione;
5-(7-{[(cyclopropylmethyl)amino]methyl}-1-fluoro-3-hydroxy-5,6,7,8-tetrahydronaphthalen-2-yl)-1λ⁶,2,5-thiadiazolidine-1,1,3-trione;
5-(7-{[(cyclobutylmethyl)amino]methyl}-1-fluoro-3-hydroxy-5,6,7,8-tetrahydronaphthalen-2-yl)-1λ⁶,2,5-thiadiazolidine-1,1,3-trione;
5-[(7R,8R)-7-amino-1-fluoro-3,8-dihydroxy-5,6,7,8-tetrahydronaphthalen-2-yl]-1λ⁶,2,5-thiadiazolidine-1,1,3-trione;
N-[(2R)-8-fluoro-6-hydroxy-7-(1,1,4-trioxo-1λ⁶,2,5-thiadiazolidin-2-yl)-1,2,3,4-tetrahydronaphthalen-2-yl]acetamide;
5-(1-fluoro-3-hydroxy-7-{[(2-hydroxyethyl)amino]methyl}-5,6,7,8-tetrahydronaphthalen-2-yl)-1λ⁶,2,5-thiadiazolidine-1,1,3-trione;
5-[(7S)-7-(aminomethyl)-1-fluoro-3-hydroxy-5,6,7,8-tetrahydronaphthalen-2-yl]-1λ⁶,2,5-thiadiazolidine-1,1,3-trione;
5-[(7R)-7-(aminomethyl)-1-fluoro-3-hydroxy-5,6,7,8-tetrahydronaphthalen-2-yl]-1λ⁶,2,5-thiadiazolidine-1,1,3-trione;
5-{(7R,8R)-1-fluoro-3,8-dihydroxy-7-[(3-methylbutyl)amino]-5,6,7,8-tetrahydronaphthalen-2-yl}-1λ⁶,2,5-thiadiazolidine-1,1,3-trione;
5-[(2S)-2-(aminomethyl)-4-fluoro-6-hydroxy-2,3-dihydro-1H-inden-5-yl]-1λ⁶,2,5-thiadiazolidine-1,1,3-trione;
5-[(2R)-2-(aminomethyl)-4-fluoro-6-hydroxy-2,3-dihydro-1H-inden-5-yl]-1λ⁶,2,5-thiadiazolidine-1,1,3-trione;
5-{(7R)-7-[(5-amino-4,4-difluoropentyl)amino]-1-fluoro-3-hydroxy-5,6,7,8-tetrahydronaphthalen-2-yl}-1λ⁶,2,5-thiadiazolidine-1,1,3-trione;
5-[(7R)-7-(butylamino)-1-fluoro-3-hydroxy-5,6,7,8-tetrahydronaphthalen-2-yl]-1λ⁶,2,5-thiadiazolidine-1,1,3-trione;
5-{(6S,7S)-1-fluoro-3,6-dihydroxy-7-[(3-methylbutyl)amino]-5,6,7,8-tetrahydronaphthalen-2-yl}-1λ⁶,2,5-thiadiazolidine-1,1,3-trione;
   and a pharmaceutically acceptable salt thereof.

In some embodiments, a compound disclosed herein is formulated as a pharmaceutically acceptable composition comprising a disclosed compound and a pharmaceutically acceptable carrier.

Also disclosed herein is a method of treating cancer in a patient in need thereof, comprising administering to the patient an effective amount of a compound disclosed herein in combination with an additional therapeutic agent. In some embodiments, the additional therapeutic agent is an immunotherapeutic agent. For example, in some embodiments, the immunotherapeutic agent is selected from the group consisting of an anti-PD-1 antibody, an anti-PD-L1 antibody and an anti-CTLA-4 antibody.

For example, disclosed herein is a method of treating cancer in a patient in need thereof, comprising administering to the patient an effective amount of a compound disclosed herein.

Further provided herein is a method of treating type-2 diabetes in a patient in need thereof, comprising administering to the patient an effective amount of a compound disclosed herein.

Disclosed herein, for example, is a method of treating and/or controlling obesity in a patient in need thereof, comprising administering to the patient an effective amount of a compound disclosed herein.

For example, disclosed herein is a method of inhibiting further weight gain in an overweight or obese patient in need thereof, comprising administering to the patient an effective amount of a compound disclosed herein.

Further disclosed herein is a method of treating a metabolic disease in a patient in need thereof, comprising administering to the patient an effective amount of a compound disclosed herein.

In some embodiments, the method comprises the treatment of cancer. In some embodiments, the cancer comprises pancreatic cancer, breast cancer, multiple myeloma, melanoma, or a cancer of the secretory cells. In some embodiments, the method comprises the treatment of a metabolic disease. In some embodiments, the metabolic disease comprises non-alcoholic steatohepatitis (NASH), non-alcoholic fatty liver disease (NAFLD), liver fibrosis, obesity, type-2 diabetes, heart disease, atherosclerosis, arthritis, cystinosis, phenylketonuria, proliferative retinopathy, metabolic syndrome or Kearns-Sayre disease.

Also disclosed herein is a composition for use in treating cancer in a patient in need thereof, wherein the composition comprises a compound disclosed herein, in combination with an additional therapeutic agent. In some embodiments, the additional therapeutic agent is an immunotherapeutic agent. For example, in some embodiments, the immunotherapeutic agent is selected from the group consisting of an anti-PD-1 antibody, an anti-PD-L1 antibody and an anti-CTLA-4 antibody.

For example, disclosed herein is a composition for use in treating cancer in a patient in need thereof, wherein the composition comprises a compound disclosed herein.

Further provided herein is a composition for use in treating type-2 diabetes in a patient in need thereof, wherein the composition comprises a compound disclosed herein.

Disclosed herein, for example, is a composition for use in treating and/or controlling obesity in a patient in need thereof, wherein the composition comprises a compound disclosed herein.

For example, disclosed herein is a composition for use in inhibiting further weight gain in an overweight or obese patient in need thereof, wherein the composition comprises a compound disclosed herein.

Further disclosed herein is a composition for use in treating a metabolic disease in a patient in need thereof, wherein the composition comprises a compound disclosed herein.

In some embodiments, the cancer comprises pancreatic cancer, breast cancer, multiple myeloma, melanoma, or a cancer of the secretory cells. In some embodiments, the metabolic disease comprises non-alcoholic steatohepatitis (NASH), non-alcoholic fatty liver disease (NAFLD), liver fibrosis, obesity, type-2 diabetes, heart disease, atherosclerosis, arthritis, cystinosis, phenylketonuria, proliferative retinopathy, metabolic syndrome or Kearns-Sayre disease.

### BRIEF DESCRIPTION OF THE SEQUENCE LISTING

Incorporated herein by reference in its entirety is a Sequence Listing entitled, CLS-023WO_ABV12579WOO1 SEQ ID List_ST25.txt”, comprising SEQ ID NO: 1 through SEQ ID NO: 3, which includes the amino acid sequence disclosed herein. The Sequence Listing has been submitted herewith in ASCII text format via EFS. The Sequence Listing was first created on Sep. 9, 2021 and is 7,306 bytes in size.

### DETAILED DESCRIPTION

The present disclosure is directed, at least in part, to compounds, compositions, and methods for the inhibition of protein tyrosine phosphatase, e.g., protein tyrosine phosphatase non-receptor type 2 (PTPN2) and/or protein tyrosine phosphatase non-receptor type 1 ((PTPN1), also known as protein tyrosine phosphatase-1B (PTPIB)).

### Definitions

### Chemical Definitions

Definitions of specific functional groups and chemical terms are described in more detail below: The chemical elements are identified in accordance with the Periodic Table of the Elements. CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers. Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ, of Notre Dame Press, Notre Dame, IN 1972). The disclosure additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

“Isotopically enriched variant” as used herein refers to a disclosed compound having one or more isotopic substitutions, wherein one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, hydrogen (H) may be in any isotopic form, including ¹H, ²H (D or deuterium), and ³H (T or tritium); carbon (C) may be in any isotopic form, including ¹²C, ¹³C, and ¹⁴C; oxygen (O) may be in any isotopic form, including ¹⁶O and ¹⁸O; and the like. For example, an isotopically enriched variant as disclosed herein may have one or more hydrogen atoms replaced with deuterium.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example, “C₁₋₆alkyl” or “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present disclosure.

“Alkyl” refers to a radical of a straight-chain or branched saturated hydrocarbon group having from 1 to 20 carbon atoms (“C₁₋₂₀alkyl” or “C₁-C₂₀ alkyl”). In some embodiments, an alkyl group has 1 to 12 carbon atoms (“C₁₋₁₂alkyl” or “C₁-C₁₂ alkyl”). In some embodiments, an alkyl group has 1 to 8 carbon atoms (“C₁₋₈alkyl” or “C₁-C₈ alkyl”). In some embodiments, an alkyl group has 1 to 6 carbon atoms (“C₁₋₆alkyl” or “C₁-C₆ alkyl”). In some embodiments, an alkyl group has 1 to 5 carbon atoms (“C₁₋₄alkyl” or “C₁-C₈ alkyl”). In some embodiments, an alkyl group has 1 to 4 carbon atoms (“C₁₋₄alkyl” or “C₁-C₄alkyl”). In some embodiments, an alkyl group has 1 to 3 carbon atoms (“C₁₋₃alkyl” or “C₁-C₈ alkyl”). In some embodiments, an alkyl group has 1 to 2 carbon atoms (“C₁₋₂alkyl” or “C₁-C₂ alkyl”). In some embodiments, an alkyl group has 1 carbon atom (“C₁ alkyl”). In some embodiments, an alkyl group has 2 to 6 carbon atoms (“C₂₋₆alkyl” or “C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents. 1 to 3 substituents, or 1 substituent. In certain embodiments, the alkyl group is unsubstituted C₁₋₁₀ alkyl (e.g., CH₃). In certain embodiments, the alkyl group is substituted C₁₋₆ alkyl. Common alkyl abbreviations include Me (—CH₃). Et (—CH₂CH₃), iPr (—CH (CH₃)₂), nPr (—CH₂CH₂CH₃), n-Bu (—CH₂CH₂CH₂CH₃), or i-Bu (—CH₂CH₂CH₃)₂).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present disclosure. The term “alkenylene,” by itself or as part of another substituent, means, unless otherwise stated, a divalent radical derived from an alkene. An alkylene group may be described as, e.g., a 1-6-membered alkylene, wherein the term “membered” refers to the non-hydrogen atoms within the moiety.

“Alkenyl” refers to a radical of a straight-chain or branched hydrocarbon group having from 2 to 20 carbon atoms, one or more carbon-carbon double bonds, and no triple bonds (“C₂₋₂₀ alkenyl” or “C₂-C₂₀ alkenyl”). In some embodiments, an alkenyl group has 2 to 10 carbon atoms (“C₂₋₁₀alkenyl” or “C₂-C₁₀ alkenyl”). In some embodiments, an alkenyl group has 2 to 8 carbon atoms (“C₂₋₈alkenyl” or “C₂-C₈ alkenyl”). In some embodiments, an alkenyl group has 2 to 6 carbon atoms (“C₂₋₆alkenyl” or “C₂-C₆ alkenyl”). In some embodiments, an alkenyl group has 2 to 5 carbon atoms (“C₂₋₅alkenyl” or “C₂-C₅ alkenyl”). In some embodiments, an alkenyl group has 2 to 4 carbon atoms (“C₂₋₄alkenyl” or “C₂-C₄ alkenyl”). In some embodiments, an alkenyl group has 2 to 3 carbon atoms (“C₂₋₃alkenyl” or “C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, e.g., unsubstituted (an “unsubstituted alkenyl”) or substituted (a “substituted alkenyl”) with one or more substituents, e.g., from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₆ alkenyl.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a 6-10-membered aryl, wherein the term “membered” refers to the non-hydrogen ring atoms within the moiety. Aryl groups include, but are not limited to, phenyl, naphthyl, indenyl, and tetrahydronaphthyl. Each instance of an aryl group may be independently optionally substituted, e.g., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

In certain embodiments, an aryl group is substituted with one or more of groups selected from halo, C₁-C₈ alkyl, halo-C₁-C₈ alkyl, haloxy-C₁-C₈ alkyl, cyano, hydroxy, alkoxy C₁-C₈ alkyl, and amino.

Examples of representative substituted aryls include the following

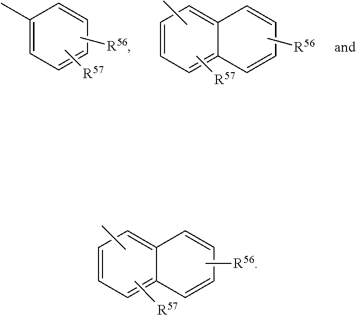

wherein one of R⁵⁶ and R⁵⁷ may be hydrogen and at least one of R⁵⁶ and R⁵⁷ is each independently selected from C₁-C₈ alkyl, halo-C₁-C₈ alkyl, 4-10 membered heterocyclyl, alkanoyl, alkoxy-C₁-C₈ alkyl, heteroaryloxy, alkylamino, arylamino, heteroarylamino, NR⁵⁸COR⁵⁹, NR⁵⁸SOR⁵⁹NR⁵⁸SO₂R⁵⁹, C(O)Oalkyl, C(O)Oaryl, CONR⁵⁸R⁵⁹, CONR⁵⁸OR⁵⁹, NR⁵⁸R⁵⁹, SO₂NR⁵⁸R⁵⁹, S-alkyl, S(O)-alkyl, S(O)₂-alkyl, S-aryl, S(O)-aryl, S(O₂)-aryl; or R⁵⁶ and R⁵⁷ may be joined to form a cyclic ring (saturated or unsaturated) from 5 to 8 atoms, optionally containing one or more heteroatoms selected from the group N, O, or S.

Other representative aryl groups having a fused heterocyclyl group include the following:

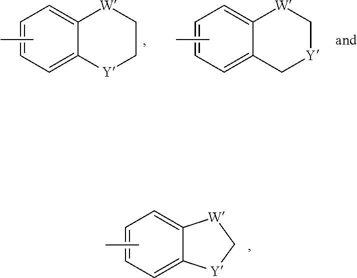

wherein each W′ is selected from C(R⁶⁶)₂, NR⁶⁶, O, and S; and each Y′ is selected from carbonyl, NR⁶⁶, O and S; and R⁶⁶ is independently hydrogen, C₁-C₈ alkyl, C₃-C₁₀ cycloalkyl, 4-10 membered heterocyclyl, C₆-C₁₀ aryl, and 5-10 membered heteroaryl.

An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

“Halo” or “halogen,” independently or as part of another substituent, mean, unless otherwise stated, a fluorine (F), chlorine (CI), bromine (Br), or iodine (I) atom. The term “halide” by itself or as part of another substituent, refers to a fluoride, chloride, bromide, or iodide atom. In certain embodiments, the halo group is either fluorine or chlorine.

Additionally, terms such as “haloalkyl” are meant to include monohaloalkyl and polyhaloalkyl. For example, the term “halo-C₁-C₆ alkyl” includes, but is not limited to, fluoromethyl, difluoromethyl, trifluoromethyl, 2,2,2-trifluoroethyl, 4-chlorobutyl, 3-bromopropyl, and the like.

The term “heteroalkyl,” by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S(O)—CH₃, —S(O)₂—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O—CH₃, —NR^{B}R^{C}, or the like, it will be understood that the terms heteroalkyl and —CH₂O—CH₃ or —NR^{B}R^{C} are not redundant or mutually exclusive. Rather, the specific heteroalkyl groups are recited to add clarity. Thus, the term “heteroalkyl” should not be interpreted herein as excluding specific heteroalkyl groups, such as —CH₂O—CH₃, —NR^{B}R^{C}, or the like.

Similarly, the term “heteroalkylene,” by itself or as part of another substituent, means, unless otherwise stated, a divalent radical derived from heteroalkyl, as exemplified, but not limited by, —CH₂O— and —CH₂CH₂O—. A heteroalkylene group may be described as, e.g., a 2-7-membered heteroalkylene, wherein the term “membered” refers to the non-hydrogen atoms within the moiety. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10) membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10) membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following formulae:

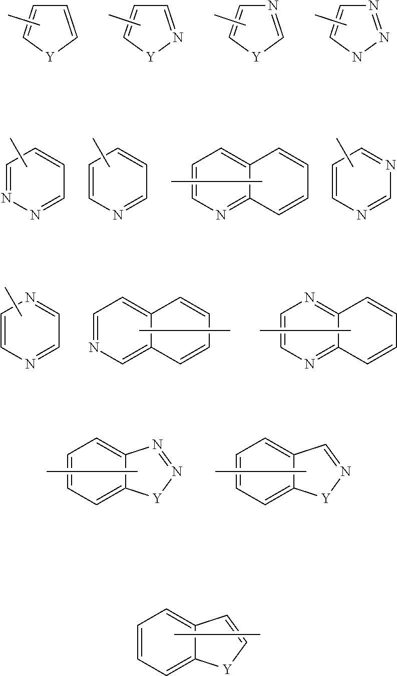

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Cycloalkyl” refers to a radical of a non-aromatic cyclic hydrocarbon group having from 3 to 10 ring carbon atoms (“C₃₋₁₀cycloalkyl” or “C₃-C₁₀ cycloalkyl”) and zero heteroatoms in the non-aromatic ring system. In some embodiments, a cycloalkyl group has 3 to 8 ring carbon atoms (“C₃₋₈cycloalkyl” or “C₃-C₈cycloalkyl”). In some embodiments, a cycloalkyl group has 3 to 6 ring carbon atoms (“C₃₋₆cycloalkyl” or “C₃-C₆ cycloalkyl”). In some embodiments, a cycloalkyl group has 5 to 10 ring carbon atoms (“C₅₋₁₀ cycloalkyl” or “C₅-C₁₀ cycloalkyl”). A cycloalkyl group may be described as, e.g., a 4-7-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₅), cyclooctenyl (C₈), cubanyl (C₈), bicyclo[1.1.1]pentanyl (C₅), bicyclo[2,2,2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, e.g., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

In some embodiments. “cycloalkyl” is a monocyclic, saturated cycloalkyl group having from 3 to 10 ring carbon atoms (“C₃₋₁₀cycloalkyl” or “C₃-C₁₀ cycloalkyl”). In some embodiments, a cycloalkyl group has 3 to 8 ring carbon atoms (“C₃₋₈cycloalkyl” or “C₃-C₈ cycloalkyl”). In some embodiments, a cycloalkyl group has 3 to 6 ring carbon atoms (“C₃₋₆cycloalkyl” or “C₃-C₆ cycloalkyl”). In some embodiments, a cycloalkyl group has 5 to 6 ring carbon atoms (“C₅₋₆cycloalkyl” or “C₅-C₆ cycloalkyl”). In some embodiments, a cycloalkyl group has 5 to 10 ring carbon atoms (“C₅₋₁₀cycloalkyl” or “C₅-C₁₀ cycloalkyl”). Examples of C₅-C₆ cycloalkyl groups include cyclopentyl (C₅) and cyclohexyl (C₅). Examples of C₃-C₆ cycloalkyl groups include the aforementioned C₅-C₆ cycloalkyl groups as well as cyclopropyl (C₃) and cyclobutyl (C₄). Examples of C₃-C₈ cycloalkyl groups include the aforementioned C₃-C₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is substituted C₃-C₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (˜5-10) membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

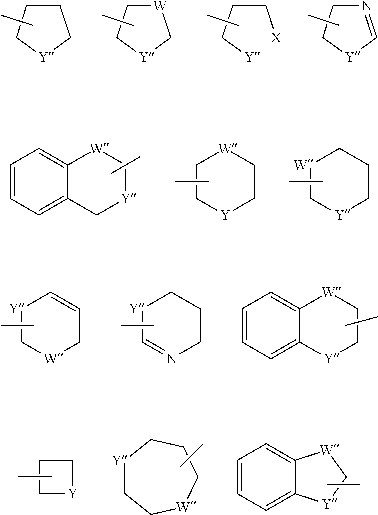

wherein each W″ is selected from CR⁶⁷, C(R⁶⁷)₂, NR⁶⁷, O, and S; and each Y″ is selected from NR⁶⁷, O, and S; and R⁶⁷ is independently hydrogen, C₁-C₈ alkyl, C₃-C₁₀ cycloalkyl, 4-10 membered heterocyclyl, C₆-C₁₀ aryl, and 5-10-membered heteroaryl. These heterocyclyl rings may be optionally substituted with one or more groups selected from the group consisting of acyl, acylamino, acyloxy, alkoxy, alkoxycarbonyl, alkoxycarbonylamino, amino, substituted amino, aminocarbonyl (e.g., amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.
“Nitrogen-containing heterocyclyl” group means a 4- to 7-membered non-aromatic cyclic group containing at least one nitrogen atom, for example, but without limitation, morpholine, piperidine (e.g, 2-piperidinyl, 3-piperidinyl and 4-piperidinyl), pyrrolidine (e.g, 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.
“Amino” refers to the radical —NR⁷⁰R⁷¹, wherein R⁷⁰ and R⁷¹ are each independently hydrogen, C₁-C₈ alkyl, C₃-C₁₀ cycloalkyl, 4-10 membered heterocyclyl, C₆-C₁₀ aryl, and 5-10-membered heteroaryl. In some embodiments, amino refers to NH₂.
“Cyano” refers to the radical —CN.
“Hydroxy” or “hydroxyl” refers to the radical —OH.

In some embodiments one or more of the nitrogen atoms of a disclosed compound if present are oxidized to the corresponding N-oxide.

Alkyl, alkenyl, alkynyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

A “counterion” or “anionic counterion” is a negatively charged group associated with a cationic quaternary amino group in order to maintain electronic neutrality. Exemplary counterions include halide ions (e.g. F⁻, Cl⁻, Br⁻, I⁻), NO₃⁻, ClO₄⁻, OH⁻, H₂PO₄⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present disclosure contain both basic and acidic functionalities that allow the compounds to be converted into either base or acid addition salts. Other pharmaceutically acceptable carriers known to those of skill in the art are suitable for the present disclosure. Salts tend to be more soluble in aqueous or other protonic solvents that are the corresponding free base forms. In other cases, the preparation may be a lyophilized powder in a first buffer, e.g., in 1 mM-50 mM histidine, 0.1%-2% sucrose, 2%-7% mannitol at a pH range of 4.5 to 5.5, that is combined with a second buffer prior to use.

Thus, the compounds of the present disclosure may exist as salts, such as with pharmaceutically acceptable acids. The present disclosure includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

### Other Definitions

“Treating” or “treatment” includes preventing or delaying the onset of the symptoms, complications, or biochemical indicia of a disease, alleviating or ameliorating the symptoms or arresting or inhibiting further development of the disease, condition, or disorder. “Treating” or “treatment” includes any effect, e.g., lessening, reducing, modulating, or eliminating, that results in the improvement of the condition, disease, disorder and the like. For example certain methods herein treat cancer by decreasing or reducing or preventing the occurrence, growth, metastasis, or progression of cancer or decreasing a symptom of cancer. The term “treating” and conjugations thereof, include prevention of an injury, pathology, condition, or disease (e.g., preventing the development of one or more symptoms of a disease, disorder, or condition described herein).

An “effective amount” is an amount sufficient to accomplish a stated purpose (e.g., achieve the effect for which it is administered, treat a disease, reduce enzyme activity, increase enzyme activity, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

A “reduction” of a symptom or symptoms (and grammatical equivalents of this phrase) means decreasing of the severity or frequency of the symptom(s), or elimination of the symptom(s).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme, e.g., a protein tyrosine phosphatase, e.g., protein tyrosine phosphatase non-receptor type 2 (PTPN2) or protein tyrosine phosphatase non-receptor type 1 (PTPN1).

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a protein tyrosine phosphatase, e.g., protein tyrosine phosphatase non-receptor type 2 (PTPN2) or protein tyrosine phosphatase non-receptor type 1 (PTPN1). Thus, inhibition may include, at least in part, partially or totally decreasing stimulation, decreasing or reducing activation, or inactivating, desensitizing, or down-regulating signal transduction or enzymatic activity or the amount of a protein tyrosine phosphatase, e.g., protein tyrosine phosphatase non-receptor type 2 (PTPN2) or protein tyrosine phosphatase non-receptor type 1 (PTPN1).

“Patient” or “subject” in need thereof refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is a parrot. In some embodiments, a patient is livestock animal. In some embodiments, a patient is a mammal. In some embodiments, a patient is a cat. In some embodiments, a patient is a horse. In some embodiments, a patient is bovine. In some embodiments, a patient is a canine. In some embodiments, a patient is a feline. In some embodiments, a patient is an ape. In some embodiments, a patient is a monkey. In some embodiments, a patient is a mouse. In some embodiments, a patient is an experimental animal. In some embodiments, a patient is a rat. In some embodiments, a patient is a hamster. In some embodiments, a patient is a test animal. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

“Disease”, “disorder” or “condition” refers to a state of being or health status of a patient or subject capable of being treated with a compound, pharmaceutical composition, or method provided herein. In some embodiments, the compounds and methods described herein comprise reduction or elimination of one or more symptoms of the disease, disorder, or condition, e.g., through administration of a compound disclosed herein, a pharmaceutically acceptable salt thereof, or a pharmaceutical composition comprising a compound disclosed herein, or a pharmaceutically acceptable salt thereof, and a pharmaceutically acceptable carrier.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water. NaCl, normal saline solutions, lactated Ringer's solution, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a compound or composition described herein is administered at the same time, just prior to, or just after the administration of one or more additional therapies (e.g., anti-cancer agent, chemotherapeutic, or immunotherapeutic agent). The compounds or compositions described herein can be administered alone or can be coadministered to the patient. Coadministration is meant to include simultaneous or sequential administration of the compound or composition individually or in combination (more than one compound or agent). Thus, the preparations can also be combined, when desired, with other active substances (e.g., to reduce metabolic degradation).

The term “PTPN2” as used herein refers to protein tyrosine phosphatase non-receptor type 2. The term “PTPN1” refers to protein tyrosine phosphatase non-receptor type 1 (PTPN1), also known as protein tyrosine phosphatase-1B (PTPIB),

### Compounds

Disclosed herein, for example, is a compound represented by Formula (I):

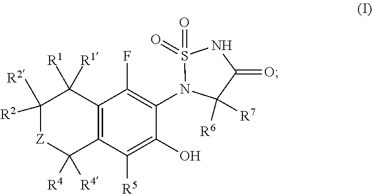

or a pharmaceutically acceptable salt thereof, wherein:
Z is selected from the group consisting of C(R³)(R^{3′}), a bond and N(R⁸);
R¹ is selected from the group consisting of hydrogen, deuterium, halogen, -hydroxyl, C₁₋₆alkyl, C₃₋₆cycloalkyl and —O—C₁₋₆alkyl;
   wherein C₁₋₆alkyl, C₃₋₆cycloalkyl and —O—C₁₋₆alkyl may optionally be substituted on one or more available carbons by one, two, three or more substituents each independently selected from R^{g};
R^{1′} is selected from the group consisting of hydrogen and deuterium;
R² is selected from the group consisting of hydrogen, hydroxyl, —C₁₋₆alkyl, —C₂₋₆alkenyl, —O—C₁₋₆alkyl, —NH₂, —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{e})₃, —C₁₋₆alkylene-N(R^{a})—C₁₋₆alkyl, —C₁₋₆alkylene-N(R^{a})—C₁₋₆alkylene-C₃₋₆cycloalkyl, —C₁₋₆alkylene-N(R^{a})(R^{b}), —C₁₋₆alkylene-N(R^{a})—C(O)—O—C₁₋₆alkyl, —N(R^{a})—(C═N(R^{b}))—C₁₋₆alkyl, —S(O)_{w}—C₁₋₆alkyl, —C(O)—N(R^{a})—C₁₋₆alkyl, —N(R^{a})—C(O)—C₁₋₆alkyl, —N(R^{a})—S(O)_{w}—C₁₋₆alkyl, —O—C(O)—N(R^{a})—C₁₋₆alkyl, —O—C(O)—N(R^{a})-phenyl, —N(R³)—C(O)—O—C₁₋₆alkyl, C₃₋₆cycloalkyl, —C₁₋₆alkylene-C₃₋₆cycloalkyl, —O—C₁₋₆alkylene-C₃₋₆cycloalkyl, 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl, —N(R^{a})—C₁₋₆alkylene-5-6 membered heteroaryl and —N(R^{a})—C₁₋₆alkylene-phenyl;
   wherein —C₁₋₆alkyl, —C₂₋₆alkenyl, —O—C₁₋₆alkyl, —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{e})₃, —C₁₋₆alkylene-N(R^{a})—C₁₋₆alkyl, —C₁₋₆alkylene-N(R^{a})—C₁₋₆alkylene-C₃₋₆cycloalkyl, —N(R^{a})—(C═N(R^{b}))—C₁₋₆alkyl, —S(O)_{w}—C₁₋₆alkyl, —C(O)—N(R^{a})—C₁₋₆alkyl, —N(R^{a})—C(O)—C₁₋₆alkyl, —O—C(O)—N(R^{a})—C₁₋₆alkyl, —O—C(O)—N(R^{a})-phenyl, —N(R^{a})—C(O)—O—C₁₋₆alkyl, C₃₋₆cycloalkyl, —C₁₋₆alkylene-C₃₋₆cycloalkyl, —O—C₁₋₆alkylene-C₃₋₆cycloalkyl, 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl, —N(R^{a})—C₁₋₆alkylene-5-6 membered heteroaryl and —N(R^{a})—C₁₋₆alkylene-phenyl may optionally be substituted on one or more available carbons by one, two, three or more substituents each independently selected from R^{g};
   wherein if 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl or —N(R^{a})—C₁₋₆alkylene-5-6 membered heteroaryl contains a substitutable ring nitrogen atom, that ring nitrogen atom may optionally be substituted by R^{h}; and
   wherein if Z is C(H)(R³), then R² is not —CH₂—CH₃;
R^{2′} is selected from the group consisting of hydrogen, deuterium, hydroxyl, —NR^{a}R^{b} and —N(R^{a})—N(R^{b})—C(O)-phenyl;
R³ is selected from the group consisting of hydrogen, deuterium, -hydroxyl, —C₁₋₆alkyl, —O—C₁₋₆alkyl, —O—C₁₋₆alkylene-C₃₋₆cycloalkyl, —N(R^{a})—C₁₋₆alkyl, —N(R^{a})—C₁₋₆alkylene-C₃₋₆cycloalkyl, —S(O)_{w}—C₁₋₆alkyl, —C(O)—N(R^{a})—C₁₋₆alkyl, —N(R^{a})—C(O)—C₁₋₆alkyl and —C₁₋₆alkylene-4-6 membered heterocyclyl;
   wherein —C₁₋₆alkyl, —O—C₁₋₆alkyl, —O—C₁₋₆alkylene-C₃₋₆cycloalkyl, —N(R^{a})—C₁₋₆alkyl, —N(R^{a})—C₁₋₆alkylene-C₃₋₆cycloalkyl, —S(O)_{w}—C₁₋₆alkyl, —C(O)—N(R^{a})—C₁₋₆alkyl, —N(R^{a})—C(O)—C₁₋₆alkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R^{3′} is selected from the group consisting of hydrogen and deuterium;
R⁴ is selected from the group consisting of hydrogen, halogen, C₁₋₆alkyl, C₃₋₆cycloalkyl and —C₁₋₆alkylene-4-6 membered heterocyclyl;
   wherein C₁₋₆alkyl, C₃₋₆cycloalkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R^{4′} is selected from the group consisting of hydrogen and deuterium;
R⁵ is selected from the group consisting of hydrogen, deuterium, halogen, C₁₋₆alkyl, C₃₋₆cycloalkyl and —C₁₋₆alkylene-4-6 membered heterocyclyl;
   wherein C₁₋₆alkyl, C₃₋₆cycloalkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R⁶ is selected from the group consisting of hydrogen and deuterium;
R⁷ is selected from the group consisting of hydrogen and deuterium;
R⁸ is selected from the group consisting of hydrogen and C₁₋₆alkyl;
R^{g} is independently selected for each occurrence from the group consisting of hydrogen, deuterium, halogen, hydroxyl, cyano, nitro, oxo, R^{a}R^{b}N—, R^{a}R^{b}N—C(O)—, R^{a}R^{b}N—SO_{w}—, R^{a}R^{b}N—C(O)—N(R^{a})—, C₁₋₆alkyl, C₂₋₆alkenyl, C₂₋₆alkynyl, C₃₋₆cycloalkyl, C₃₋₆cycloalkyl-C₁₋₆alkylene-, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{a})—, C₁₋₆alkyl-N(R³)—C(O)—, C₁₋₆alkyl-C(O)—N(R^{a}), C₁₋₆alkyl-N(R^{a})—C(O)—N(R^{a})—, C₁₋₆alkyl-N(R^{a})—SO_{w}—, C₃₋₆cycloalkyl-N(R^{a})—SO_{w}—, C₁₋₆alkyl-SO_{w}—N(R^{a})—, C₃₋₆cycloalkyl-SO_{w}—N(R^{a})—, C₁₋₆alkoxy-C(O)—N(R^{a})—, C₁₋₆alkyl-C(O)—N(R^{a})—C₁₋₆alkyl-, C₁₋₆alkyl-N(R^{a})—C(O)—C₁₋₆alkyl- and C₁₋₆alkoxy-C₁₋₆alkyl-;
   wherein C₁₋₆alkyl, C₂₋₆alkenyl, C₂₋₆alkynyl, C₃₋₆cycloalkyl, —C₁₋₆alkylene-C₃₋₆cycloalkyl, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)—, C₁₋₆alkyl-N(R^{a})—, C₁₋₆alkyl-N(R³)—C(O)—, C₁₋₆alkyl-C(O)—N(R^{a}), C₁₋₆alkyl-N(R^{a})—C(O)—N(R^{a})—, C₁₋₆alkyl-N(R^{a})—SO_{w}—, C₃₋₆cycloalkyl-N(R^{a})—SO_{w}—, C₁₋₆alkyl-SO_{w}—N(R^{a})—, C₃₋₆cycloalkyl-SO_{w}—N(R^{a})—, C₁₋₆alkoxy-C(O)—N(R^{a})—, C₁₋₆alkyl-C(O)—N(R^{a})—C₁₋₆alkyl-, C₁₋₆alkyl-N(R^{a})—C(O)—C₁₋₆alkyl- and C₁₋₆alkoxy-C₁₋₆alkyl- may optionally be substituted by one, two three or more substituents each independently selected from R^{P};
R^{h} is independently selected for each occurrence from the group consisting of C₁₋₆alkyl, C₃₋₆alkenyl, C₃₋₆alkynyl, C₃₋₆cycloalkyl, —C₁₋₆alkylene-C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{a}R^{b}N—C(O)— and R^{a}R^{b}N—SO₂—;
   wherein C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{a}R^{b}N—C(O)— and R^{a}R^{b}N—SO₂— may optionally be substituted by one, two three or more substituents each independently selected from R^{P};
R^{P} is independently selected for each occurrence from the group consisting of halogen, deuterium, hydroxyl, cyano, C₁₋₆alkoxy, C₃₋₆cycloalkyl, R^{a}R^{b}N—, R^{a}R^{b}N-carbonyl-, R^{a}R^{b}N—SO₂—, and R^{a}R^{b}N-carbonyl-N(R^{a})—;
R^{a} and R^{b} are independently selected, for each occurrence, from the group consisting of hydrogen and C₁₋₆alkyl; wherein C₁₋₆alkyl may optionally be substituted by one or more substituents each independently selected from the group consisting of halogen, cyano, oxo and hydroxyl;
or R^{a} and R^{b} together with the nitrogen to which they are attached form a 4-6 membered heterocyclyl, wherein 4-6 membered heterocyclyl may optionally be substituted by one or more substituents each independently selected from the group consisting of halogen, cyano, oxo and hydroxyl;
R^{c} is independently selected, for each occurrence, from the group consisting of hydroxyl, C₁₋₄alkyl and phenyl; and
w is 0, 1 or 2.

For example, a compound disclosed herein may be represented by Formula (Ia):

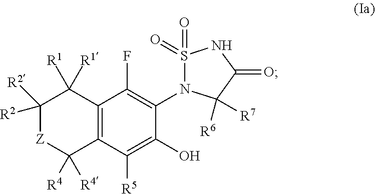

or a pharmaceutically acceptable salt thereof, wherein:
R¹ is selected from the group consisting of hydrogen, deuterium, halogen, -hydroxyl, C₁₋₆alkyl, C₃₋₆cycloalkyl and —O—C₁₋₆alkyl;
   wherein C₁₋₆alkyl, C₃₋₆cycloalkyl and —O—C₁₋₆alkyl may optionally be substituted on one or more available carbons by one, two, three or more substituents each independently selected from R^{g};
R^{1′} is selected from the group consisting of hydrogen and deuterium;
R² is selected from the group consisting of hydrogen, hydroxyl, —C₁₋₆alkyl, —C₂₋₆alkenyl, —O—C₁₋₆alkyl, —NH₂, —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{c})₃, —C₁₋₆alkylene-N(R^{a})—C₁₋₆alkyl, —C₁₋₆alkylene-N(R^{a})—C₁₋₆alkylene-C₃₋₆cycloalkyl, —C₁₋₆alkylene-N(R^{a})(R^{b}), —N(R^{a})—(C═N(R^{b}))—C₁₋₆alkyl, —S(O)_{w}—C₁₋₆alkyl, —C(O)—N(R^{a})—C₁₋₆alkyl, —N(R^{a})—C(O)—C₁₋₆alkyl, —N(R^{a})—S(O)_{w}—C₁₋₆alkyl, —O—C(O)—N(R^{a})—C₁₋₆alkyl, —O—C(O)—N(R^{a})-phenyl, —N(R^{a})—C(O)—O—C₁₋₆alkyl, C₃₋₆cycloalkyl, —C₁₋₆alkylene-C₃₋₆cycloalkyl, —O—C₁₋₆alkylene-C₃₋₆cycloalkyl, 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl, —N(R^{a})—C₁₋₆alkylene-5-6 membered heteroaryl and —N(R^{a})—C₁₋₆alkylene-phenyl;
   wherein —C₁₋₆alkyl, —C₂₋₆alkenyl, —O—C₁₋₆alkyl, —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{c})₃, —N(R^{a})—(C═N(R^{b}))—C₁₋₆alkyl, —S(O)_{w}—C₁₋₆alkyl, —C(O)—N(R^{a})—C₁₋₆alkyl, —N(R^{a})—C(O)—C₁₋₆alkyl, —O—C(O)—N(R³)—C₁₋₆alkyl, —O—C(O)—N(R^{a})-phenyl, —N(R^{a})—C(O)—O—C₁₋₆alkyl, C₃₋₆cycloalkyl, —C₁₋₆alkylene-C₃₋₆cycloalkyl, —O—C₁₋₆alkylene-C₃₋₆cycloalkyl, 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl, —N(R^{a})—C₁₋₆alkylene-5-6 membered heteroaryl and —N(R^{a})—C₁₋₆alkylene-phenyl may optionally be substituted on one or more available carbons by one, two, three or more substituents each independently selected from R^{g}; and
   wherein if 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl or —N(R^{a})—C₁₋₆alkylene-5-6 membered heteroaryl contains a substitutable ring nitrogen atom, that ring nitrogen atom may optionally be substituted by R^{h};
R^{2′} is selected from the group consisting of hydrogen, deuterium, hydroxyl, —NR^{a}R^{b} and —N(R^{a})—N(R^{b})—C(O)-phenyl;
R³ is selected from the group consisting of hydrogen, deuterium, -hydroxyl, —C₁₋₆alkyl, —O—C₁₋₆alkyl, —O—C₁₋₆alkylene-C₃₋₆cycloalkyl, —N(R^{a})—C₁₋₆alkyl, —N(R^{a})—C₁₋₆alkylene-C₃₋₆cycloalkyl, —S(O)_{w}—C₁₋₆alkyl, —C(O)—N(R^{a})—C₁₋₆alkyl, —N(R^{a})—C(O)—C₁₋₆alkyl and —C₁₋₆alkylene-4-6 membered heterocyclyl;
   wherein —C₁₋₆alkyl, —O—C₁₋₆alkyl, —O—C₁₋₆alkylene-C₃₋₆cycloalkyl, —N(R^{a})—C₁₋₆alkyl, —N(R^{a})—C₁₋₆alkylene-C₃₋₆cycloalkyl, —S(O)_{w}—C₁₋₆alkyl, —C(O)—N(R^{a})—C₁₋₆alkyl, —N(R^{a})—C(O)—C₁₋₆alkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R^{3′} is selected from the group consisting of hydrogen and deuterium;
R⁴ is selected from the group consisting of hydrogen, halogen, C₁₋₆alkyl, C₃₋₆cycloalkyl and —C₁₋₆alkylene-4-6 membered heterocyclyl;
   wherein C₁₋₆alkyl, C₃₋₆cycloalkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R⁴ is selected from the group consisting of hydrogen and deuterium;
R⁵ is selected from the group consisting of hydrogen, deuterium, halogen, C₁₋₆alkyl, C₃₋₆cycloalkyl and —C₁₋₆alkylene-4-6 membered heterocyclyl;
   wherein C₁₋₆alkyl, C₃₋₆cycloalkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R⁶ is selected from the group consisting of hydrogen and deuterium;
R⁷ is selected from the group consisting of hydrogen and deuterium;
R^{g} is independently selected for each occurrence from the group consisting of hydrogen, deuterium, halogen, hydroxyl, cyano, nitro, oxo, R^{a}R^{b}N—, R^{a}R^{b}N—C(O)—, R^{a}R^{b}N—SO_{w}—, R^{a}R^{b}N—C(O)—N(R^{a})—, C₁₋₆alkyl, C₂₋₆alkenyl, C₂₋₆alkynyl, C₃₋₆cycloalkyl, C₃₋₆cycloalkyl-C₁₋₆alkylene-, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{a})—, C₁₋₆alkyl-N(R^{a})—C(O)—, C₁₋₆alkyl-C(O)—N(R^{a}), C₁₋₆alkyl-N(R^{a})—C(O)—N(R^{a})—, C₁₋₆alkyl-N(R^{a})—SO_{w}—, C₃₋₆cycloalkyl-N(R^{a})—SO_{w}—, C₁₋₆alkyl-SO_{w}—N(R^{a})—, C₃₋₆cycloalkyl-SO_{w}—N(R^{a})—, C₁₋₆alkoxy-C(O)—N(R^{a})—, C₁₋₆alkyl-C(O)—N(R^{a})—C₁₋₆alkyl-, C₁₋₆alkyl-N(R^{a})—C(O)—C₁₋₆alkyl- and C₁₋₆alkoxy-C₁₋₆alkyl-;
   wherein C₁₋₆alkyl, C₂₋₆alkenyl, C₂₋₆alkynyl, C₃₋₆cycloalkyl, —C₁₋₆alkylene-C₃₋₆cycloalkyl, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{a})—, C₁₋₆alkyl-N(R^{a})—C(O)—, C₁₋₆alkyl-C(O)—N(R^{a}), C₁₋₆alkyl-N(R^{a})—C(O)—N(R^{a})—, C₁₋₆alkyl-N(R^{a})—SO_{w}—, C₃₋₆cycloalkyl-N(R^{a})—SO_{w}—, C₁₋₆alkyl-SO_{w}—N(R^{a})—, C₃₋₆cycloalkyl-SO_{w}—N(R^{a})—, C₁₋₆alkoxy-C(O)—N(R^{a})—, C₁₋₆alkyl-C(O)—N(R^{a})—C₁₋₆alkyl-, C₁₋₆alkyl-N(R^{a})—C(O)—C₁₋₆alkyl- and C₁₋₆alkoxy-C₁₋₆alkyl- may optionally be substituted by one, two three or more substituents each independently selected from R^{P};
R^{h} is independently selected for each occurrence from the group consisting of C₁₋₆alkyl, C₃₋₆alkenyl, C₃₋₆alkynyl, C₃₋₆cycloalkyl, —C₁₋₆alkylene-C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{a}R^{b}N—C(O)— and R^{a}R^{b}N—SO₂—;
   wherein C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{a}R^{b}N—C(O)— and R^{a}R^{b}N—SO₂— may optionally be substituted by one, two three or more substituents each independently selected from R^{P};
R^{P} is independently selected for each occurrence from the group consisting of halogen, deuterium, hydroxyl, cyano, C₁₋₆alkoxy, C₃₋₆cycloalkyl, R^{a}R^{b}N—, R^{a}R^{b}N-carbonyl-, R^{a}R^{b}N—SO₂—, and R^{a}R^{b}N-carbonyl-N(R^{a})—;
R^{a} and R^{b} are independently selected, for each occurrence, from the group consisting of hydrogen and C₁₋₆alkyl; wherein C₁₋₆alkyl may optionally be substituted by one or more substituents each independently selected from the group consisting of halogen, cyano, oxo and hydroxyl;
or R^{a} and R^{b} together with the nitrogen to which they are attached form a 4-6 membered heterocyclyl, wherein 4-6 membered heterocyclyl may optionally be substituted by one or more substituents each independently selected from the group consisting of halogen, cyano, oxo and hydroxyl;
R^{c} is independently selected, for each occurrence, from the group consisting of hydroxyl, C₁₋₄alkyl and phenyl; and
w is 0, 1 or 2.

In some embodiments, R¹ is selected from the group consisting of hydrogen and deuterium. In some embodiments, R¹ is selected from the group consisting of hydrogen and fluorine. In some embodiments, R¹ is hydrogen. In some embodiments, R¹ is deuterium. In some embodiments, R¹ is-hydroxyl.

In various embodiments, R² is selected from the group consisting of —C₁₋₆alkylene-N(R^{a})(R^{b}) and —C₁₋₆alkylene-N(R^{a})—C₁₋₆alkyl. In some embodiments, R^{a} is hydrogen. In some embodiments, R^{b} is hydrogen. For example, R² may be selected from the group consisting of

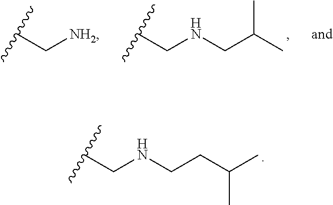

In some embodiments, R² is selected from the group consisting of

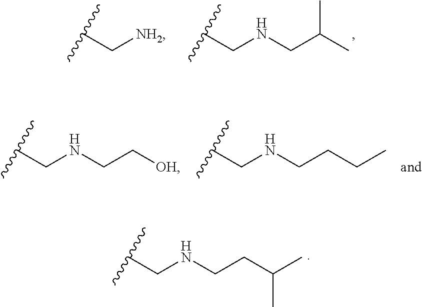

In various embodiments, R² is —C₁₋₆alkylene-N(R^{a})—C(O)—O—C₁₋₆alkyl or —N(R^{a})C(O)—C₁₋₆alkyl. In some embodiments, R² is

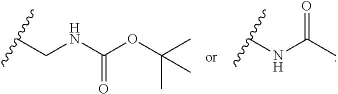

In some embodiments, R² is —C₁₋₆alkylene-N(R^{a})—C(O)—O—C₁₋₆alkyl. For example, R² may be

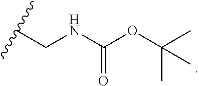 [SMILES: *CNC(=O)OC(C)(C)C]

In various embodiments, R² is selected from the group consisting of —NH₂, —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{c})₃, —N(R^{a})—C(O)—O—C₁₋₆alkyl, —N(R³)—S(O)_{w}—C₁₋₆alkyl, —N(R³)-4-6 membered heterocyclyl, —N(R^{a})—C₁₋₆alkylene-4-6 membered heterocyclyl, —N(R^{a})—C₁₋₆alkylene-5-6 membered heteroaryl and —N(R^{a})—C₁₋₆alkylene-phenyl;
wherein —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{c})₃, —N(R^{a})—C(O)—O—C₁₋₆alkyl, —N(R^{a})—S(O)_{w}—C₁₋₆alkyl, —N(R^{a})-4-6 membered heterocyclyl, —N(R³)—C₁₋₆alkylene-4-6 membered heterocyclyl, —N(R^{a})—C₁₋₆alkylene-5-6 membered heteroaryl and —N(R^{a})—C₁₋₆alkylene-phenyl may optionally be substituted on one or more available carbons by one, two, three or more substituents each independently selected from R^{g}; and
   wherein if —N(R^{a})-4-6 membered heterocyclyl, —N(R^{a})—C₁₋₆alkylene-4-6 membered heterocyclyl or —N(R^{a})—C₁₋₆alkylene-5-6 membered heteroaryl contains a substitutable ring nitrogen atom, that ring nitrogen atom may optionally be substituted by R^{h};

In various embodiments, R² is —N(R^{a})—C₁₋₈alkyl, wherein R² may optionally be substituted by one, two, three or more substituents each independently selected from R^{g}. In some embodiments, R² may optionally be substituted by one, two, three or more substituents each independently selected from the group consisting of deuterium, fluorine, hydroxyl, NR^{a}R^{b}—, and C₁₋₆alkoxy. In some embodiments, R^{a} and R^{b} are each hydrogen. In some embodiments, R² is —N(R^{a})—C₅alkyl. For example, R² may be selected from the group consisting of

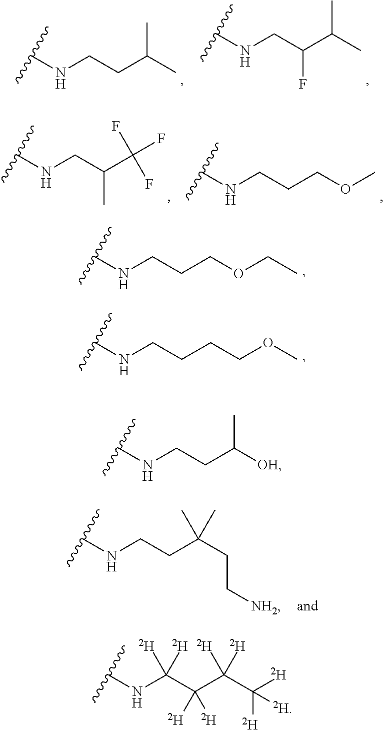

In certain embodiments, R² may be selected from the group consisting of

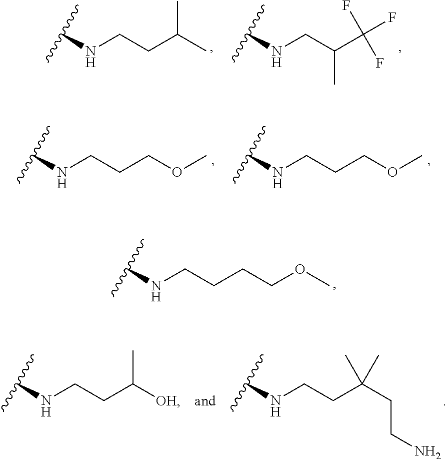

In some other embodiments, R² is —N(R^{a})—C₁₋₈alkyl, wherein R² may optionally be substituted by one, two, three or more substituents each independently selected from the group consisting of fluorine, hydroxyl, cyano and C₁₋₆alkoxy, wherein C₁₋₆alkoxy may optionally be substituted by one, two or three fluorines. In some embodiments, R^{a} is hydrogen. For example, R² may be selected from the group consisting of

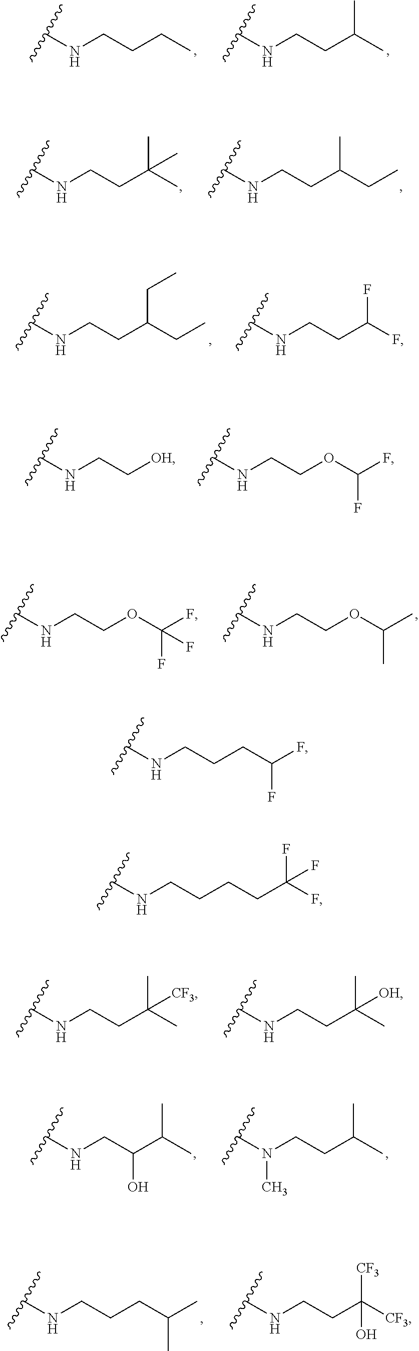

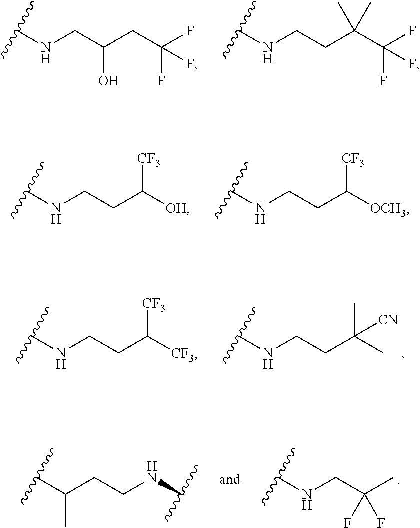

For example, R² may be selected from the group consisting of

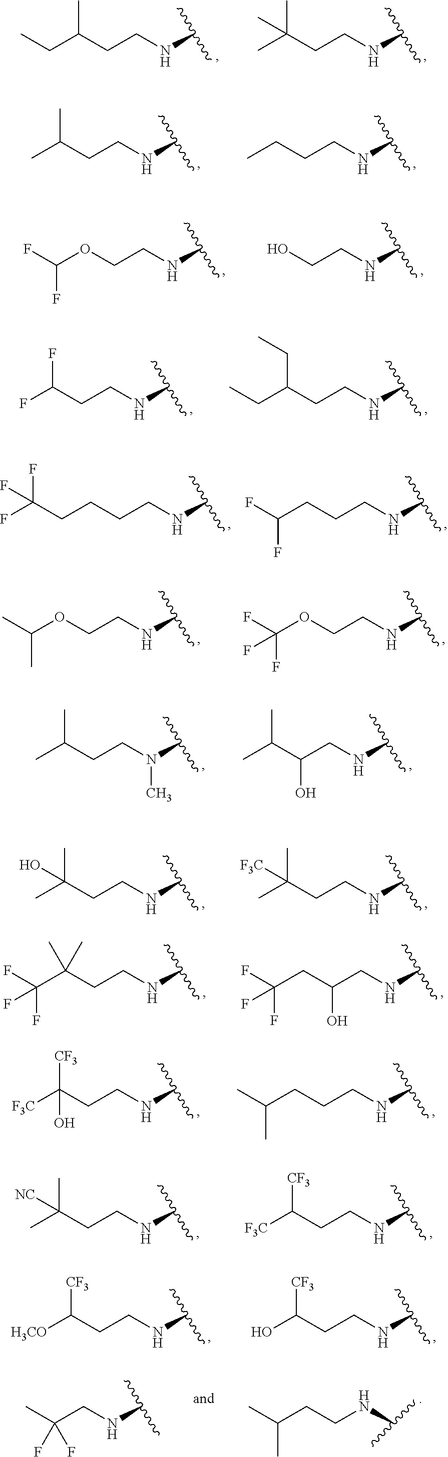

In some embodiments, R² is selected from the group consisting of

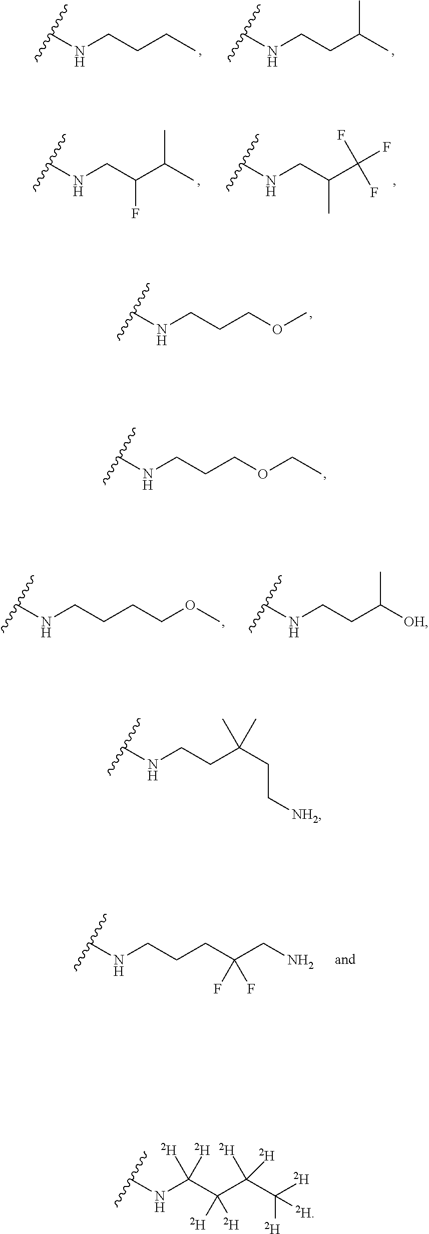

In some embodiments, R² is

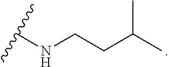 [SMILES: *NCCC(C)C]

In some embodiments, when R² is

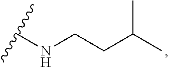 [SMILES: *NCCC(C)C]

one or more of R¹, R^{1′}, R^{2′}, R³, R^{3′}, R⁴, R^{4′}, R⁵, R⁶, and R⁷ is not hydrogen. In some embodiments, R² is

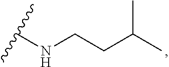 [SMILES: *NCCC(C)C]

and R⁵ is fluorine. In some embodiments, R² is

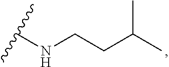 [SMILES: *NCCC(C)C]

and one or more of R¹, R^{1′}, R^{2′}, R³, R^{3′}, R⁴, R^{4′}, R⁵, R⁶, and R⁷ is deuterium. In certain embodiments, R² is

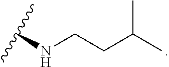

In various embodiments, R² is —N(R^{a})—C(O)—O—C₁₋₆alkyl, for example, R² may be represented by

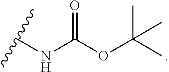 [SMILES: *NC(=O)OC(C)(C)C]

In certain embodiments, R² may be represented by or

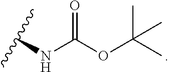

In various embodiments, R² is —N(R^{a})—S(O)_{w}—C₁₋₆alkyl. For example, R² may be

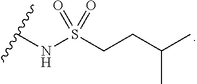 [SMILES: *NS(=O)(=O)CCC(C)C]

In various embodiments, R² is —N(R^{a})—C₁₋₆alkylene-C₃₋₆cycloalkyl, wherein R² may optionally be substituted by one, two, three or more substituents each independently selected from R^{g}. In some embodiments, R² may optionally be substituted by one, two, three or more substituents each independently selected from the group consisting of fluorine, cyano, and C₁₋₆alkyl, wherein C₁₋₆alkyl may optionally be substituted by one, two or three substituents selected from the group consisting of fluorine, hydroxyl, and —NH₂. For example, R² may be selected from the group consisting of

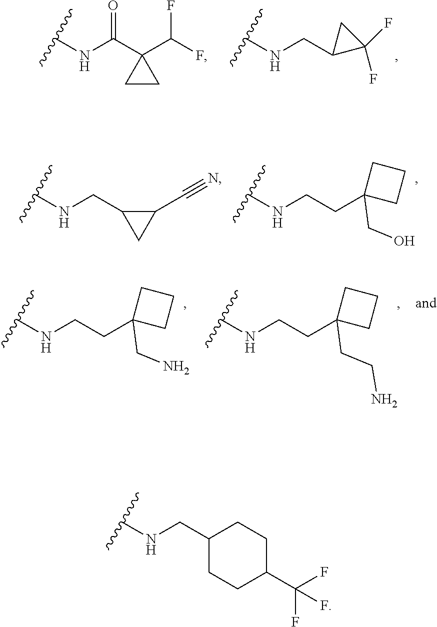

In certain embodiments, R² may be selected from the group consisting of

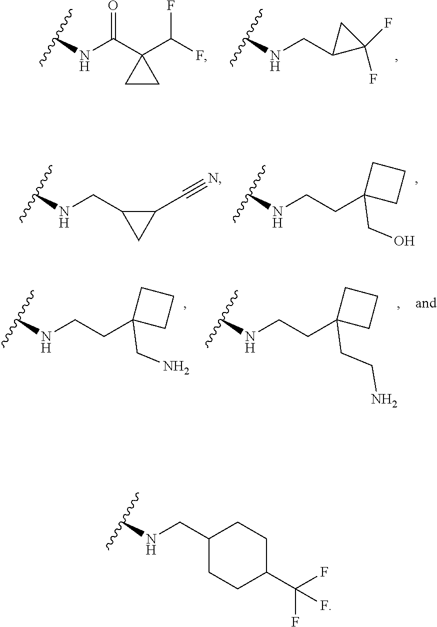

In some other embodiments, R² may be selected from the group consisting of

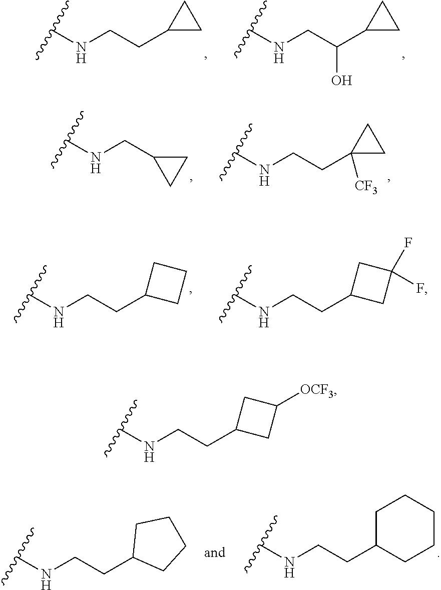

In some further embodiments, R² may be selected from the group consisting of

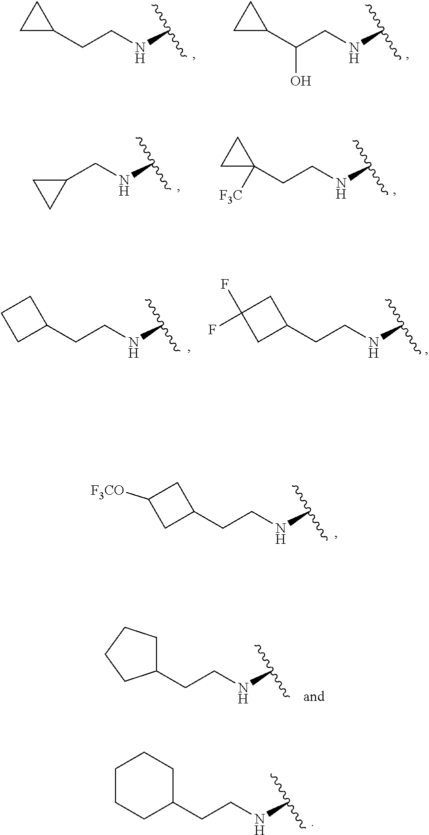

In some embodiments, R² is —N(R^{a})—C₁₋₆alkylene-C₃₋₆cycloalkyl or —C₁₋₆alkylene-N(R^{a})—C₁₋₆alkylene-C₃₋₆cycloalkyl, wherein R² may optionally be substituted by one, two, three or more substituents each independently selected from R^{g}. In some embodiments, R² is —N(R^{a})—C₁₋₆alkylene-C₃₋₆cycloalkyl or —C₁₋₆alkylene-N(R^{a})—C₁₋₆alkylene-C₃₋₆cycloalkyl, wherein R² may optionally be substituted by one, two, three or more substituents each independently selected from the group consisting of fluorine, cyano, and C₁₋₆alkyl, wherein C₁₋₆alkyl may optionally be substituted by one, two or three substituents selected from the group consisting of fluorine, hydroxyl, and —NH₂. In some embodiments, R² is selected from the group consisting of

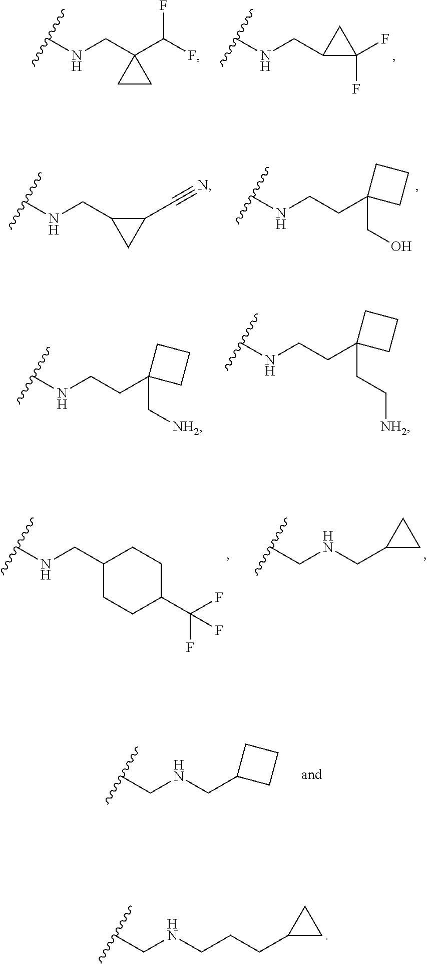

In various embodiments, R² is —N(R^{a})—C₁₋₆alkylene-4-6 membered heterocyclyl. In some embodiments, R² may optionally be substituted by one or two C₁₋₆alkyl. For example, R² may be selected from the group consisting of

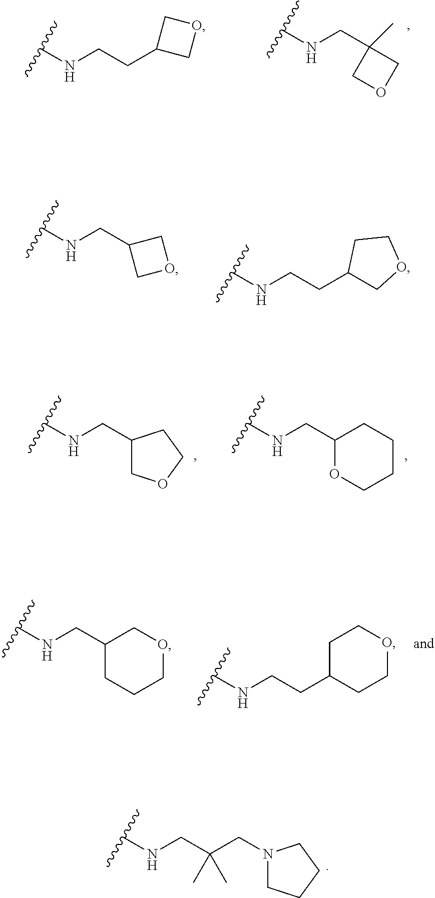

In certain embodiments, R² may be selected from the group consisting of

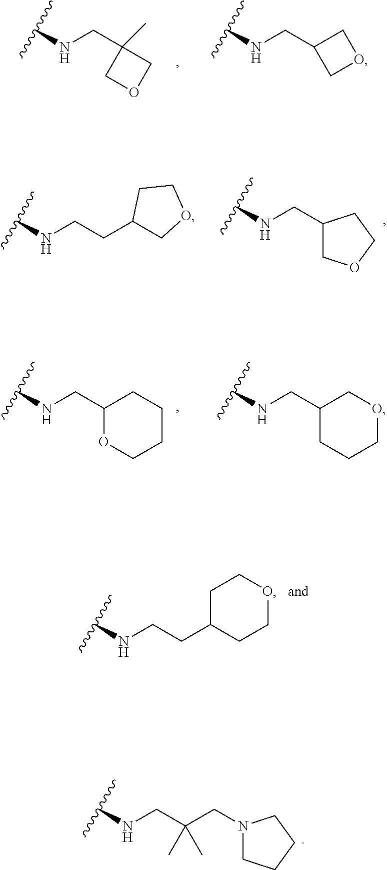

In some other embodiments, R² may be represented by

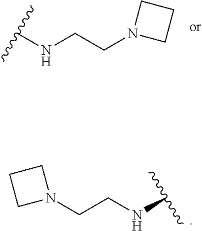

In various embodiments, R² is —N(R^{a})—C₁₋₆alkylene-5-6 membered heteroaryl, wherein R² may optionally be substituted by one, two, three or more substituents each independently selected from R^{g}. In some embodiments, R² may optionally be substituted by one or two C₁₋₆alkyl. For example, R² may be selected from the group consisting of

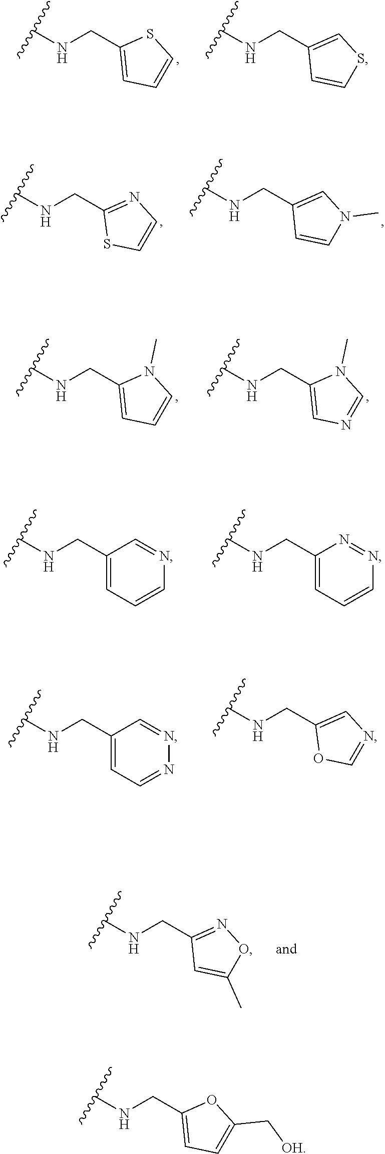

In certain embodiments, R² may be selected from the group consisting of

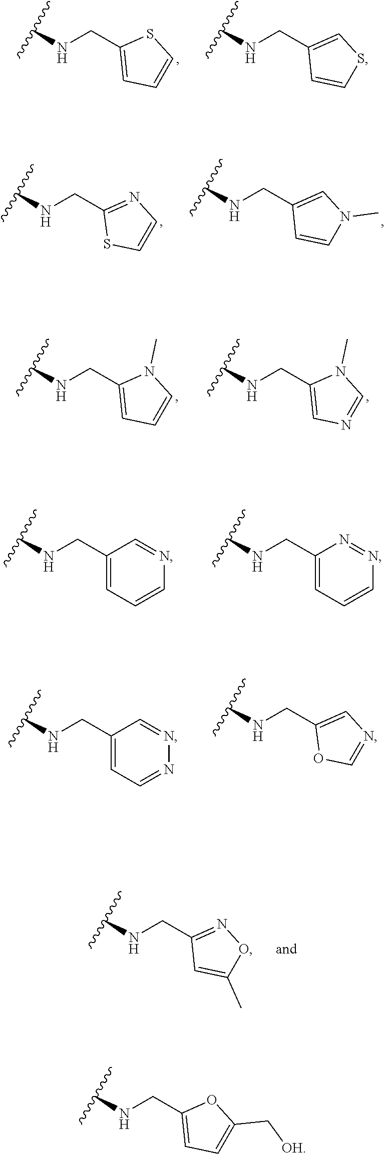

In some other embodiments, R² may be selected from the group consisting of

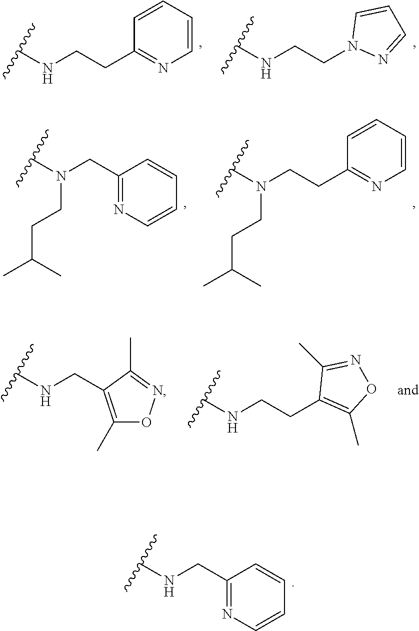

In some further embodiments, R² may be selected from the group consisting of

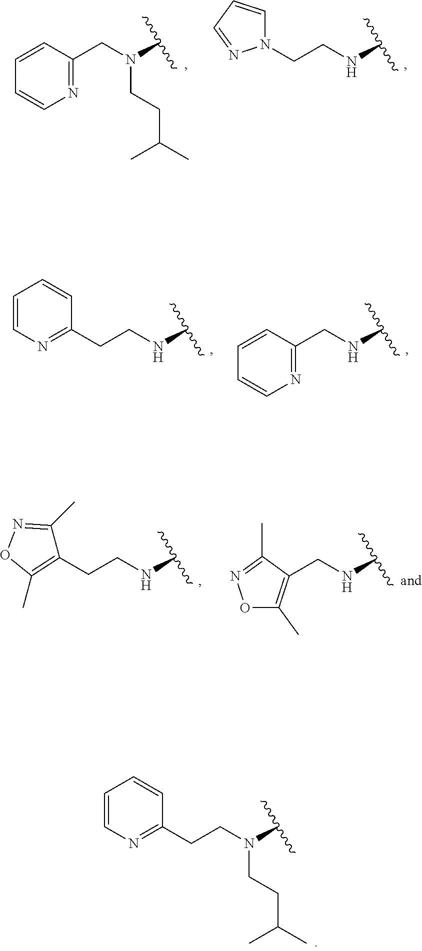

In various embodiments, R² is selected from the group consisting of hydrogen and hydroxyl. In some embodiments, R² is hydrogen. In some embodiments, R² is hydroxyl. In some embodiments, R² is —NH₂.

In certain embodiments, R² is —O—C₁₋₆alkyl; wherein R² may optionally be substituted by one, two, three or more substituents each independently selected from R^{g}. In some embodiments, R² may optionally be substituted by one, two, three or more substituents each independently selected for each occurrence from the group consisting of fluorine, hydroxyl, C₁₋₆alkoxy and R^{a}R^{b}N—. For example, R² may be selected from the group consisting of: —OCH₃,

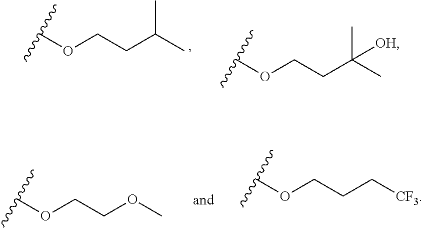

In certain embodiments, R² is —N(R^{a})—C₁₋₆alkylene-phenyl, wherein R² may optionally be substituted by one, two, three or more substituents each independently selected from R^{g}. In some embodiments, R² may optionally be substituted by one, two or three fluorine atoms. For example, R² may be represented by

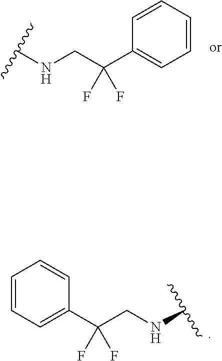

In some embodiments, R² is —C₁₋₆alkylene-C₃₋₆cycloalkyl. For example, R² may be represented by

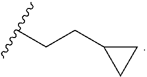 [SMILES: *CCC1CC1]

In other embodiments, R² is —O—C₁₋₆alkylene-C₃₋₆cycloalkyl, wherein R² may optionally be substituted by one, two, three or more substituents each independently selected from R^{g}. In some embodiments, R² may optionally be substituted by one, two or three fluorine atoms. For example, R² may be selected from the group consisting of

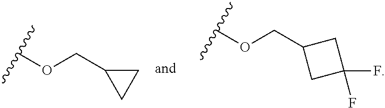 [SMILES: *OCC1CC1 ; *OCC1CC(F)(F)C1]

In other embodiments, R² is 4-6 membered heterocyclyl, wherein 4-6 membered heterocyclyl may optionally be substituted by one, two, three or more substituents each independently selected from R⁸, and wherein if 4-6 membered heterocyclyl contains a substitutable ring nitrogen atom, that ring nitrogen atom may optionally be substituted by R^{h}. For example, R² may be pyrrolidinyl, wherein pyrrolidinyl may optionally be substituted by R^{h}. In some embodiments, R^{h} is selected from the group consisting of C₁₋₆alkyl, —C₁₋₆alkylene-C₃₋₆cycloalkyl and C₃₋₆cycloalkyl-S(O)₂—. For example, R² may be selected from the group consisting of

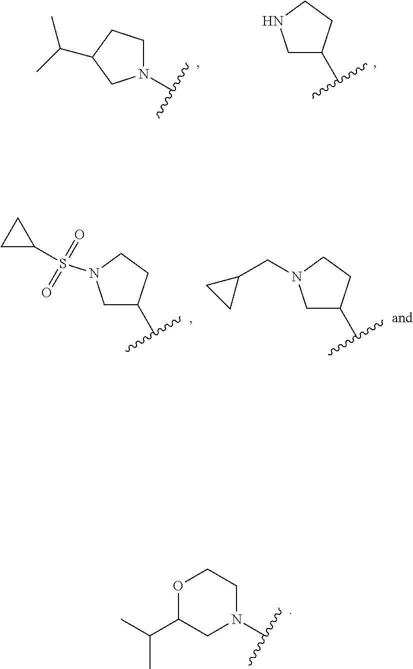

In further embodiments, R² is —O—C(O)—N(R^{a})-phenyl. For example, R² may be represented by

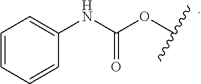 [SMILES: *OC(=O)Nc1ccccc1]

In some embodiments, R² is —N(R^{a})—C₁₋₆alkylene-Si(R^{c})₃. For example, R² may be selected from the group consisting of

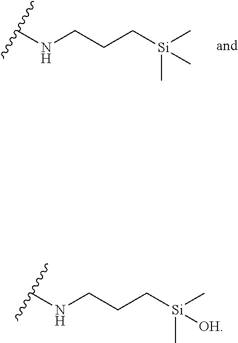

For example, R² may be selected from the group consisting of

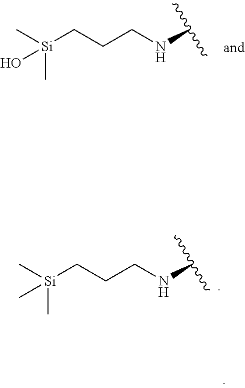

In some embodiments, R² is —N(R^{a})—(C═N(R^{b}))—C₁₋₆alkyl. For example, R² may represented by

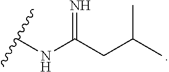 [SMILES: *NC(=N)CC(C)C]

In some embodiments, R² is —N(R^{a})—C₃₋₆cycloalkyl, wherein R² may optionally be substituted by one, two, three or more substituents each independently selected from R^{g}. For example, R² may be represented by

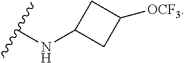 [SMILES: *NC1CC(OC(F)(F)F)C1]

For example, R² may be represented as

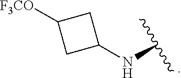

In some embodiments, R^{2′} is selected from the group consisting of hydrogen, deuterium, and hydroxyl. In other embodiments, R^{2′} is selected from the group consisting of hydrogen and —NH₂.

In some embodiments, R³ is selected from the group consisting of hydrogen and deuterium. In some embodiments, R³ is -hydroxyl.

In some other embodiments, R³ is selected from the group consisting of —O—C₁₋₆alkyl and —O—C₁₋₆alkylene-C₃₋₆cycloalkyl, wherein R³ may optionally be substituted by one, two, three or more substituents each independently selected from R^{g}. For example, R³ may be selected from the group consisting of —OCH₃,

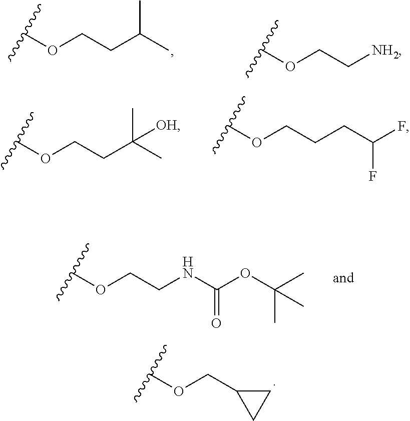

In some further embodiments, R³ is selected from the group consisting of —N(R^{a})—C₁₋₆alkyl and —N(R^{a})—C₁₋₆alkylene-C₃₋₆cycloalkyl, wherein R³ may optionally be substituted by one, two, three or more substituents each independently selected from R^{g}. For example, R³ may be selected from the group consisting of,

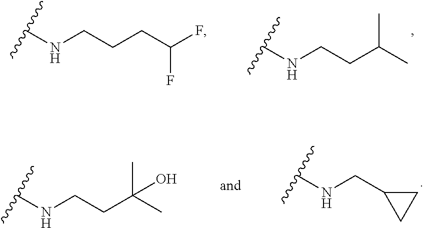

In some embodiments, R⁴ is selected from the group consisting of hydrogen and methyl. In some embodiments, R^{4′} is hydrogen.

In some embodiments, R⁵ is selected from the group consisting of hydrogen and fluorine.

In some embodiments, R^{a}, when present, is hydrogen.

Further disclosed herein is a compound is represented by Formula I(c) or a pharmaceutically acceptable salt thereof:

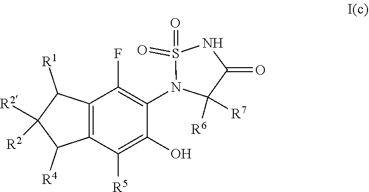

wherein
R¹ is selected from the group consisting of hydrogen, deuterium, halogen, -hydroxyl, C₁₋₆alkyl, C₃₋₆cycloalkyl and —O—C₁₋₆alkyl;
   wherein C₁₋₆alkyl, C₃₋₆cycloalkyl and —O—C₁₋₆alkyl may optionally be substituted on one or more available carbons by one, two, three or more substituents each independently selected from R^{g};
R² is selected from the group consisting of hydrogen, hydroxyl, —C₁₋₆alkyl, —C₂₋₆alkenyl, —O—C₁₋₆alkyl, —NH₂, —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{c})₃, —C₁₋₆alkylene-N(R^{a})—C₁₋₆alkyl, —C₁₋₆alkylene-N(R^{a})—C₁₋₆alkylene-C₃₋₆cycloalkyl, —C₁₋₆alkylene-N(R^{a})(R^{b}), —N(R^{a})—(C═N(R^{b}))—C₁₋₆alkyl, —S(O)_{w}—C₁₋₆alkyl, —C(O)—N(R^{a})—C₁₋₆alkyl, —N(R^{a})—C(O)—C₁₋₆alkyl, —N(R^{a})—S(O)_{w}—C₁₋₆alkyl, —O—C(O)—N(R^{a})—C₁₋₆alkyl, —O—C(O)—N(R^{a})-phenyl, —N(R^{a})—C(O)—O—C₁₋₆alkyl, C₃₋₆cycloalkyl, —C₁₋₆alkylene-C₃₋₆cycloalkyl, —O—C₁₋₆alkylene-C₃₋₆cycloalkyl, 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl, —N(R^{a})—C₁₋₆alkylene-5-6 membered heteroaryl and —N(R^{a})—C₁₋₆alkylene-phenyl;
   wherein —C₁₋₆alkyl, —C₂₋₆alkenyl, —O—C₁₋₆alkyl, —N(R^{a})—C₁₋₈alkyl, —N(R^{a})—C₃₋₆cycloalkyl, —N(R^{a})—C₁₋₆alkylene-C₃₋₆cycloalkyl, —N(R^{a})—C₁₋₆alkylene-Si(R^{c})₃, —N(R^{a})—(C═N(R^{b}))—C₁₋₆alkyl, —S(O)_{w}—C₁₋₆alkyl, —C(O)—N(R^{a})—C₁₋₆alkyl, —N(R^{a})—C(O)—C₁₋₆alkyl, —O—C(O)—N(R^{a})—C₁₋₆alkyl, —O—C(O)—N(R^{a})-phenyl, —N(R^{a})—C(O)—O—C₁₋₆alkyl, C₃₋₆cycloalkyl, —C₁₋₆alkylene-C₃₋₆cycloalkyl, —O—C₁₋₆alkylene-C₃₋₆cycloalkyl, 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl, —N(R^{a})—C₁₋₆alkylene-5-6 membered heteroaryl and —N(R^{a})—C₁₋₆alkylene-phenyl may optionally be substituted on one or more available carbons by one, two, three or more substituents each independently selected from R^{g}; and
   wherein if 5-6 membered heteroaryl, 4-6 membered heterocyclyl, —N(R^{a})-4-6 membered heterocyclyl, —C₁₋₆alkylene-4-6 membered heterocyclyl, —O—C₁₋₆alkylene-4-6 membered heterocyclyl, —N(R^{a})—C₁₋₆alkylene-4-6 membered heterocyclyl or —N(R^{a})—C₁₋₆alkylene-5-6 membered heteroaryl contains a substitutable ring nitrogen atom, that ring nitrogen atom may optionally be substituted by R^{h};
R^{2′} is selected from the group consisting of hydrogen, deuterium, hydroxyl, —NR^{a}R^{b} and —N(R^{a})—N(R^{b})—C(O)-phenyl;
R⁴ is selected from the group consisting of hydrogen, halogen, C₁₋₆alkyl, C₃₋₆cycloalkyl and —C₁₋₆alkylene-4-6 membered heterocyclyl;
   wherein C₁₋₆alkyl, C₃₋₆cycloalkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R⁵ is selected from the group consisting of hydrogen, deuterium, halogen, C₁₋₆alkyl, C₃₋₆cycloalkyl and —C₁₋₆alkylene-4-6 membered heterocyclyl;
   wherein C₁₋₆alkyl, C₃₋₆cycloalkyl and —C₁₋₆alkylene-4-6 membered heterocyclyl may optionally be substituted on one or more available carbons by one, two, three or more substituents each independently selected from R^{g}; and
   wherein if —C₁₋₆alkylene-4-6 membered heterocyclyl contains a substitutable ring nitrogen atom, that ring nitrogen atom may optionally be substituted by R^{h};
R⁶ is selected from the group consisting of hydrogen and deuterium;
R⁷ is selected from the group consisting of hydrogen and deuterium;
R^{g} is independently selected for each occurrence from the group consisting of hydrogen, deuterium, halogen, hydroxyl, cyano, nitro, oxo, R^{a}R^{b}N—, R^{a}R^{b}N—C(O)—, R^{a}R^{b}N—SO_{w}—, R^{a}R^{b}N—C(O)—N(R^{a})—, C₁₋₆alkyl, C₂₋₆alkenyl, C₂₋₆alkynyl, C₃₋₆cycloalkyl, C₃₋₆cycloalkyl-C₁₋₆alkylene-, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{a})—, C₁₋₆alkyl-N(R^{a})—C(O)—, C₁₋₆alkyl-C(O)—N(R^{a}), C₁₋₆alkyl-N(R^{a})—C(O)—N(R^{a})—, C₁₋₆alkyl-N(R^{a})—SO_{w}—, C₃₋₆cycloalkyl-N(R^{a})—SO_{w}—, C₁₋₆alkyl-SO_{w}—N(R^{a})—, C₃₋₆cycloalkyl-SO_{w}—N(R^{a})—, C₁₋₆alkoxy-C(O)—N(R^{a})—, C₁₋₆alkyl-C(O)—N(R^{a})—C₁₋₆alkyl-, C₁₋₆alkyl-N(R^{a})—C(O)—C₁₋₆alkyl- and C₁₋₆alkoxy-C₁₋₆alkyl-;
   wherein C₁₋₆alkyl, C₂₋₆alkenyl, C₂₋₆alkynyl, C₃₋₆cycloalkyl, —C₁₋₆alkylene-C₃₋₆cycloalkyl, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{a})—, C₁₋₆alkyl-N(R^{a})—C(O)—, C₁₋₆alkyl-C(O)—N(R^{a}), C₁₋₆alkyl-N(R^{a})—C(O)—N(R^{a})—, C₁₋₆alkyl-N(R^{a})—SO_{w}—, C₃₋₆cycloalkyl-N(R^{a})—SO_{w}—, C₁₋₆alkyl-SO_{w}—N(R^{a})—, C₃₋₆cycloalkyl-SO_{w}—N(R^{a})—, C₁₋₆alkoxy-C(O)—N(R^{a})—, C₁₋₆alkyl-C(O)—N(R^{a})—C₁₋₆alkyl-, C₁₋₆alkyl-N(R^{a})—C(O)—C₁₋₆alkyl- and C₁₋₆alkoxy-C₁₋₆alkyl- may optionally be substituted by one, two three or more substituents each independently selected from R^{P};
R^{h} is independently selected for each occurrence from the group consisting of C₁₋₆alkyl, C₃₋₆alkenyl, C₃₋₆alkynyl, C₃₋₆cycloalkyl, —C₁₋₆alkylene-C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{a}R^{b}N—C(O)— and R^{a}R^{b}N—SO₂—;
   wherein C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{a}R^{b}N—C(O)— and R^{a}R^{b}N—SO₂— may optionally be substituted by one, two three or more substituents each independently selected from R^{P};
R^{P} is independently selected for each occurrence from the group consisting of halogen, deuterium, hydroxyl, cyano, C₁₋₆alkoxy, C₃₋₆cycloalkyl, R^{a}R^{b}N—, R^{a}R^{b}N-carbonyl-, R^{a}R^{b}N—SO₂—, and R^{a}R^{b}N-carbonyl-N(R^{a})—;
R^{a} and R^{b} are independently selected, for each occurrence, from the group consisting of hydrogen and C₁₋₆alkyl; wherein C₁₋₆alkyl may optionally be substituted by one or more substituents each independently selected from the group consisting of halogen, cyano, oxo and hydroxyl;
or R^{a} and R^{b} together with the nitrogen to which they are attached form a 4-6 membered heterocyclyl, wherein 4-6 membered heterocyclyl may optionally be substituted by one or more substituents each independently selected from the group consisting of halogen, cyano, oxo and hydroxyl;
R^{c} is independently selected, for each occurrence, from the group consisting of hydroxyl, C₁₋₄alkyl and phenyl; and
   w is 0, 1 or 2.

In some embodiments, R² is selected from the group consisting of —C₁₋₆alkylene-N(R^{a})—C₁₋₆alkyl, —C₁₋₆alkylene-N(R^{a})(R^{b}) and —C₁₋₆alkylene-N(R^{a})—C₁₋₆alkylene-C₃₋₆cycloalkyl, wherein R² may optionally be substituted by one, two, three or more substituents each independently selected from R^{g}. For example, R² may be selected from the group consisting of

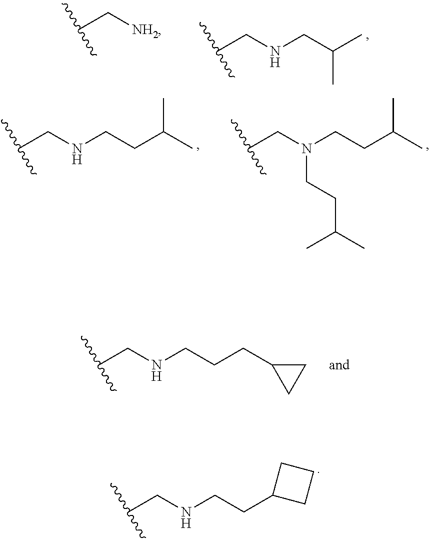

Further disclosed herein is a compound is represented by Formula I(d) or a pharmaceutically acceptable salt thereof:

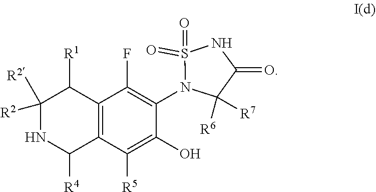

In some embodiments, R² is C₁₋₆alkyl.

In other embodiments, R⁴ is hydrogen. In further embodiments, R⁵ is selected from the group consisting of hydrogen and fluorine. In certain embodiments, R⁶ is hydrogen. In yet further embodiments, R⁷ is hydrogen.

Also disclosed herein is a compound represented by Formula (II):

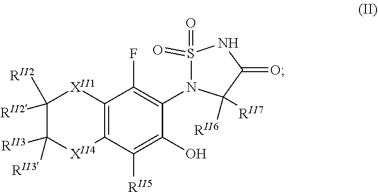

or a pharmaceutically acceptable salt thereof, wherein:
X^{II1} is selected from the group consisting of O and C(R^{II1})(R^{II1′});
X^{II4} is selected from the group consisting of O and C(R^{II4})(R^{II4′});
   wherein at least one of X^{II1} and X^{II4} is O;
R^{II1} and R^{II1′} are each independently selected from the group consisting of hydrogen, halogen, -hydroxyl, C₁₋₆alkyl, C₂₋₆alkenyl, C₂₋₆alkynyl and C₃₋₆cycloalkyl;
   wherein C₁₋₆alkyl, C₂₋₆alkenyl, C₂₋₆alkynyl and C₃₋₆cycloalkyl may optionally be substituted on one or more available carbons by one, two, three or more substituents each independently selected from R^{IIg};
R^{II2} is selected from the group consisting of hydrogen, C₁₋₆alkyl, C₂₋₆alkenyl, C₂₋₆alkynyl, —O—C₁₋₆alkyl, —NH(R^{IIa}), —N(R^{IIa})—C₁₋₆alkyl, —N(R^{IIa})—C₁₋₆alkylene-C₃₋₆cycloalkyl, —S(O)₂—C₁₋₆alkyl, —C(O)—N(R^{IIa})—C₁₋₆alkyl, —N(R^{IIa})—C(O)—C₁₋₆alkyl, —O—C(O)—N(R^{IIa})—C₁₋₆alkyl, —N(R^{IIa})—C(O)—O—C₁₋₆alkyl, C₃₋₆cycloalkyl, phenyl, 5-6 membered heteroaryl, 4-6 membered heterocyclyl, —C₁₋₆alkylene-C₃₋₆cycloalkyl, —C₁₋₆alkylene-phenyl, —C₁₋₆alkylene-5-6 membered heteroaryl, —C₁₋₆alkylene-4-6 membered heterocyclyl, —O—C₁₋₆alkylene-C₃₋₆cycloalkyl, —N(R^{IIa})-4-6 membered heterocyclyl, —O—C₁₋₆alkylene-4-6 membered heterocyclyl, —N(R^{IIa})—C₁₋₆alkylene-4-6 membered heterocyclyl, —N(R^{IIa})—C₁₋₆alkylene-5-6 membered heteroaryl and —N(R^{IIa})—C₁₋₆alkylene-phenyl;
   wherein C₁₋₆alkyl, C₂₋₆alkenyl, C₂₋₆alkynyl, —O—C₁₋₆alkyl, —N(R^{IIa})—C₁₋₆alkyl, —N(R^{IIa})—C₁₋₆alkylene-C₃₋₆cycloalkyl, —S(O)₂—C₁₋₆alkyl, —C(O)—N(R^{IIa})—C₁₋₆alkyl, —N(R^{IIa})—C(O)—C₁₋₆alkyl, —O—C(O)—N(R^{IIa})—C₁₋₆alkyl, —N(R^{IIa})—C(O)—O—C₁₋₆alkyl, C₃₋₆cycloalkyl, phenyl, 5-6 membered heteroaryl, 4-6 membered heterocyclyl, —C₁₋₆alkylene-C₃₋₆cycloalkyl, —C₁₋₆alkylene-phenyl, —C₁₋₆alkylene-5-6 membered heteroaryl, —C₁₋₆alkylene-4-6 membered heterocyclyl, —O—C₁₋₆alkylene-C₃₋₆cycloalkyl, —N(R^{IIa})-4-6 membered heterocyclyl, —O—C₁₋₆alkylene-4-6 membered heterocyclyl, —N(R^{IIa})—C₁₋₆alkylene-4-6 membered heterocyclyl, —N(R^{IIa})—C₁₋₆alkylene-5-6 membered heteroaryl and —N(R^{IIa})—C₁₋₆alkylene-phenyl may optionally be substituted on one or more available carbons by one, two, three or more substituents each independently selected from R^{IIg};
   wherein if 5-6 membered heteroaryl, 4-6 membered heterocyclyl, —C₁₋₆alkylene-5-6 membered heteroaryl, —C₁₋₆alkylene-4-6 membered heterocyclyl, —N(R^{IIa})-4-6 membered heterocyclyl, —O—C₁₋₆alkylene-4-6 membered heterocyclyl, —N(R^{IIa})—C₁₋₆alkylene-4-6 membered heterocyclyl, or —N(R^{IIa})—C₁₋₆alkylene-5-6 membered heteroaryl contains a substitutable ring nitrogen atom, that ring nitrogen atom may optionally be substituted by R^{IIh}; and
   wherein if R^{II2} is —O—C₁₋₆alkyl, —N(R^{IIa})—C₁₋₆alkyl, —N(R^{IIa})—C₁₋₆alkylene-C₃₋₆cycloalkyl, —S(O)₂—C₁₋₆alkyl, —N(R^{IIa})—C(O)—C₁₋₆alkyl, —O—C(O)—N(R^{IIa})—C₁₋₆alkyl, —N(R^{IIa})—C(O)—O—C₁₋₆alkyl, —O—C₁₋₆alkylene-C₃₋₆cycloalkyl, —N(R^{IIa})-4-6 membered heterocyclyl, —O—C₁₋₆alkylene-4-6 membered heterocyclyl, —N(R^{IIa})—C₁₋₆alkylene-4-6 membered heterocyclyl, —N(R^{IIa})—C₁₋₆alkylene-5-6 membered heteroaryl or —N(R^{IIa})—C₁₋₆alkylene-phenyl; then X^{II1} is C(R^{II1})(R^{II1′}) and X^{II4} is O;
R^{II2′} is selected from the group consisting of hydrogen, C₁₋₆alkyl, C₂₋₆alkenyl, C₂₋₆alkynyl, C₃₋₆cycloalkyl, phenyl, 5-6 membered heteroaryl, 4-6 membered heterocyclyl, —C₁₋₆alkylene-C₃₋₆cycloalkyl, —C₁₋₆alkylene-phenyl, —C₁₋₆alkylene-5-6 membered heteroaryl and —C₁₋₆alkylene-4-6 membered heterocyclyl;
   wherein C₁₋₆alkyl, C₂₋₆alkenyl, C₂₋₆alkynyl. C₃₋₆cycloalkyl, phenyl, 5-6 membered heteroaryl, 4-6 membered heterocyclyl, —C₁₋₆alkylene-C₃₋₆cycloalkyl, —C₁₋₆alkylene-phenyl, —C₁₋₆alkylene-5-6 membered heteroaryl and —C₁₋₆alkylene-4-6 membered heterocyclyl may optionally be substituted on one or more available carbons by one, two, three or more substituents each independently selected from R^{IIg}; and
   wherein if 5-6 membered heteroaryl, 4-6 membered heterocyclyl, —C₁₋₆alkylene-5-6 membered heteroaryl or —C₁₋₆alkylene-4-6 membered heterocyclyl contains a substitutable ring nitrogen atom, that ring nitrogen atom may optionally be substituted by R^{IIh};
R^{II3} and R^{II3′} are each independently selected from the group consisting of hydrogen, -hydroxyl, C₁₋₆alkyl, C₂₋₆alkenyl, C₂₋₆alkynyl and C₃₋₆cycloalkyl;
   wherein C₁₋₆alkyl, C₂₋₆alkenyl, C₂₋₆alkynyl and C₃₋₆cycloalkyl may optionally be substituted on one or more available carbons by one, two, three or more substituents each independently selected from R^{IIg};
R^{II4} and R^{II4′} are each independently selected from the group consisting of hydrogen, halogen, C₁₋₆alkyl, C₂₋₆alkenyl, C₂₋₆alkynyl and C₃₋₆cycloalkyl;
   wherein C₁₋₆alkyl, C₂₋₆alkenyl, C₂₋₆alkynyl and C₃₋₆cycloalkyl may optionally be substituted on one or more available carbons by one, two, three or more substituents each independently selected from R^{IIg};
R^{II5} is selected from the group consisting of hydrogen, deuterium, halogen, C₁₋₆alkyl and C₃₋₆cycloalkyl;
   wherein C₁₋₆alkyl and C₃₋₆cycloalkyl may optionally be substituted on one or more available carbons by one, two, three or more substituents each independently selected from R^{IIg};
R^{II6} is selected from the group consisting of hydrogen and deuterium;
R^{II7} is selected from the group consisting of hydrogen and deuterium;
R^{IIg} is independently selected for each occurrence from the group consisting of hydrogen, halogen, hydroxyl, cyano, nitro, oxo, phenyl, 5-6 membered heteroaryl, R^{IIa}R^{IIb}N—, R^{IIa}R^{IIb}N—C(O)—, R^{IIa IIb}N—SO_{w}—, R^{IIa}R^{IIb}N—C(O)—N(R^{IIIa})—, C₁₋₆alkyl, C₂₋₆alkenyl, C₂₋₆alkynyl, C₃₋₆cycloalkyl, C₃₋₆cycloalkyl-C₁₋₆alkylene-, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{IIa})—, C₁₋₆alkyl-N(R^{IIa})—C(O)—, C₁₋₆alkyl-C(O)—N(R^{IIa})—, C₁₋₆alkyl-N(R^{IIa})—C(O)—N(R^{IIa})—, C₁₋₆alkyl-N(R^{IIa})—SO_{w}—, C₃₋₆cycloalkyl-N(R^{IIa})—SO_{w}—, C₁₋₆alkyl-SO_{w}—N(R^{IIa})—, C₃₋₆cycloalkyl-SO_{w}—N(R^{IIa})—, C₁₋₆alkoxy-C(O)—N(R^{IIa})—, C₁₋₆alkyl-C(O)—N(R^{IIa})—C₁₋₆alkyl-, C₁₋₆alkyl-N(R^{IIa})—C(O)—C₁₋₆alkyl- and C₁₋₆alkoxy-C₁₋₆alkyl-;
   wherein C₁₋₆alkyl, C₂₋₆alkenyl, C₂₋₆alkynyl, C₃₋₆cycloalkyl, —C₁₋₆alkylene-C₃₋₆cycloalkyl, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{IIa})—, C₁₋₆alkyl-N(R^{IIa})—C(O)—, C₁₋₆alkyl-C(O)—N(R^{IIa})—, C₁₋₆alkyl-N(R^{IIa})—C(O)—N(R^{IIa})—, C₁₋₆alkyl-N(R^{IIa})—SO_{w}—, C₃₋₆cycloalkyl-N(R^{IIa})—SO_{w}—, C₁₋₆alkyl-SO_{w}—N(R^{IIa})—, C₃₋₆cycloalkyl-SO_{w}—N(R^{IIa})—, C₁₋₆alkoxy-C(O)—N(R^{IIa})—, C₁₋₆alkyl-C(O)—N(R^{IIa})—C₁₋₆alkyl-, C₁₋₆alkyl-N(R^{IIa})—C(O)—C₁₋₆alkyl- and C₁₋₆alkoxy-C₁₋₆alkyl- may optionally be substituted by one, two three or more substituents each independently selected from R^{IIP};
R^{IIh} is independently selected for each occurrence from the group consisting of C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{IIa}R^{IIb}N—C(O)— and R^{IIa}R^{IIb}N—SO₂—;
   wherein C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{IIa}R^{IIb}N—C(O)— and R^{IIa}R^{IIb}N—SO₂— may optionally be substituted by one, two three or more substituents each independently selected from R^{IIP};
R^{IIP} is independently selected for each occurrence from the group consisting of halogen, hydroxyl, cyano, C₁₋₆alkoxy, C₃₋₆cycloalkyl, R^{IIa}R^{IIb}N—, R^{IIa}R^{IIb}N-carbonyl-, R^{IIa}R^{IIb}N—SO₂—, and R^{IIa}R^{IIb}N-carbonyl-N(R^{IIa})—;
R^{IIa} and R^{IIb} are independently selected, for each occurrence, from the group consisting of hydrogen and C₁₋₃alkyl; wherein C₁₋₃alkyl may optionally be substituted by one or more substituents each independently selected from the group consisting of halogen, cyano, oxo and hydroxyl;
or R^{IIa} and R^{IIb} together with the nitrogen to which they are attached form a 4-6 membered heterocyclyl, wherein 4-6 membered heterocyclyl may optionally be substituted by one or more substituents each independently selected from the group consisting of halogen, cyano, oxo and hydroxyl; and
w is 0, 1 or 2.

In some embodiments, R^{II5} is selected from the group consisting of hydrogen and deuterium. In some embodiments, R^{II6} is deuterium. In some embodiments, R^{II7} is deuterium. In some other embodiments, R^{II5}, R^{II6} and R^{II7} are each hydrogen.

In some embodiments, R^{II1} and R^{II1′}, when present, are each hydrogen. In some embodiments, R^{II4} and R^{II4′}, when present, are each hydrogen.

In various embodiments, a compound disclosed herein is represented by

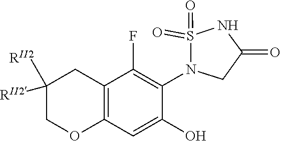

or a pharmaceutically acceptable salt thereof.

In various embodiments, R^{II2} is —NH(R^{IIa}) or —NH₂. In some embodiments, R^{IIa} is hydrogen.

In various embodiments, R^{II2} is —N(R^{IIa})—C₁₋₆alkyl, wherein R^{II2} may optionally be substituted by one, two, three or more substituents each independently selected from R^{IIg}. In some embodiments, R^{II2} may optionally be substituted by one, two, three or more substituents each independently selected from the group consisting of fluorine and C₁₋₆alkoxy, wherein C₁₋₆alkoxy may optionally be substituted by one, two or three fluorine. For example, R^{II2} may be selected from the group consisting of

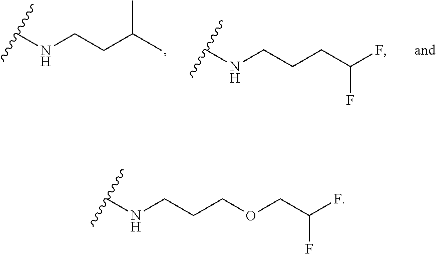

In certain embodiments, R^{II2} may be selected from the group consisting of

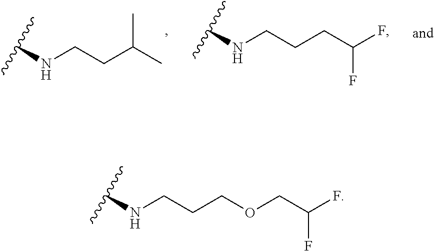

In various embodiments, R^{II2} is —N(R^{IIa})—C₁₋₆alkylene-C₃₋₆cycloalkyl, wherein R^{II2} may optionally be substituted by one, two, three or more substituents each independently selected from R^{IIg}. In some embodiments, R^{II2} may optionally be substituted by one, two, three or more substituents each independently selected from the group consisting of fluorine, C₁₋₆alkyl, and phenyl, wherein C₁₋₆alkyl may optionally be substituted by one, two or three fluorine. For example, R^{II2} may be selected from the group consisting of

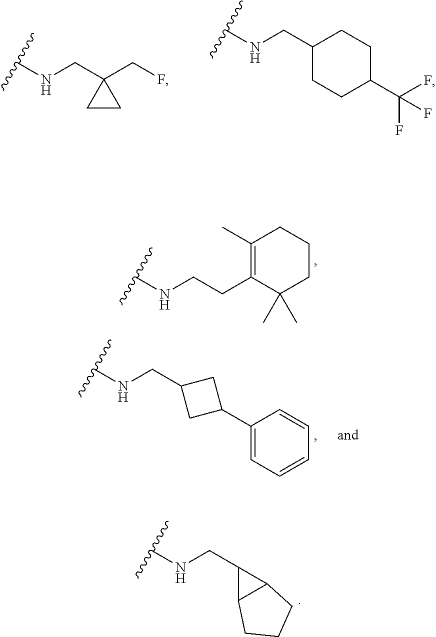 [SMILES: *NCC1(CF)CC1 ; *NCC1CCC(C(F)(F)F)CC1 ; *NCCC1=C(C)CCCC1(C)C ; *NCC1CC(c2ccccc2)C1 ; *NCC1C2CCCC21]

In certain embodiments, R^{II2} may be selected from the group consisting of

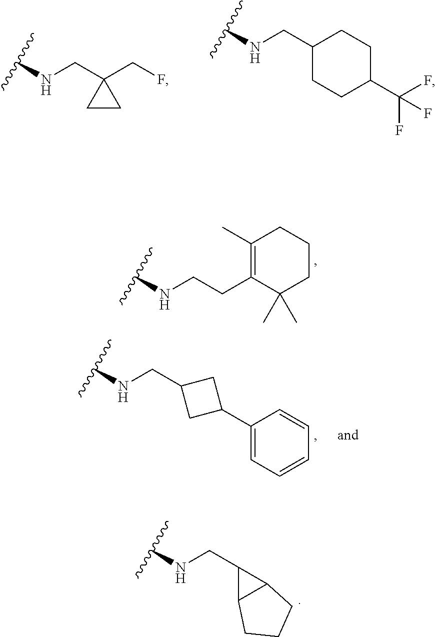

In various embodiments, R^{II2} is —N(R^{IIa})—C₁₋₆alkylene-4-6 membered heterocyclyl, wherein R^{II2} may optionally be substituted by one, two, three or more substituents each independently selected from R^{IIg}, wherein if 4-7 membered heterocyclyl contains a substitutable ring nitrogen atom, that ring nitrogen atom may optionally be substituted by R^{IIIh}. In some embodiments, R^{II2} may optionally be substituted by C₁₋₆alkoxy-C(O)—. For example, R^{II2} may be selected from the group consisting of

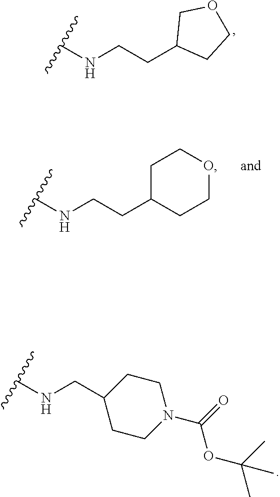 [SMILES: *NCCC1CCOC1 ; *NCCC1CCOCC1 ; "and" ; *NCC1CCN(C(=O)OC(C)(C)C)CC1]

In certain embodiments, R^{II2} may be selected from the group consisting of

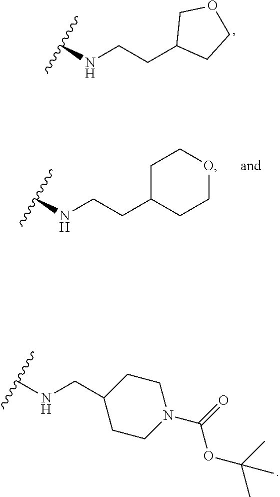

In various embodiments, R^{II2} is —N(R^{IIa})—C₁₋₆alkylene-phenyl. For example, R^{II2} may be represented by

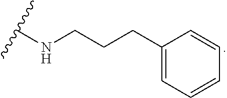 [SMILES: *NCCCc1ccccc1]

In certain embodiments, R^{II2} may be represented by

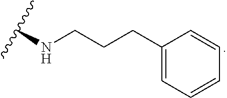

In some embodiments, R^{II2′} is hydrogen.

In some embodiments, R^{IIa}, when present, is hydrogen.

In some other embodiments, a compound disclosed herein is represented by

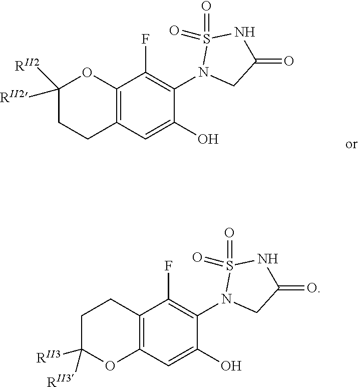

For example, R^{II2} and R^{II2′} may each be independently selected from the group consisting of hydrogen and —CH₃. For example, R^{II3} and R^{II3′} may each be independently selected from the group consisting of hydrogen and —CH₃.

Also disclosed herein is a compound represented by Formula (III):

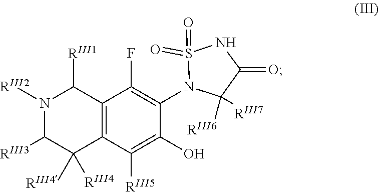

or a pharmaceutically acceptable salt thereof, wherein:
R^{III1} is selected from the group consisting of hydrogen, oxo, C₁₋₆alkyl, C₂₋₆alkenyl and C₂₋₆alkynyl;
R^{III2} is selected from the group consisting of hydrogen, C₁₋₆alkyl, C₂₋₆alkenyl, C₂₋₆alkynyl, C₃₋₆cycloalkyl, phenyl, 4-7 membered heterocyclyl, 5-6 membered heteroaryl, —C₁₋₆alkylene-C₃₋₈cycloalkyl, —C₁₋₆alkylene-phenyl, —C₁₋₆alkylene-4-7 membered heterocyclyl, —C₁₋₆alkylene-5-6 membered heteroaryl, —C(O)—C₁₋₆alkyl, —C(O)—O—C₁₋₆alkyl, —C(O)—C₁₋₆alkylene-C₃₋₈cycloalkyl, —C(O)—N(R^{IIIa})—C₁₋₆alkyl, —C(O)—N(R^{IIIa})—C₁₋₆alkylene-C₃₋₆cycloalkyl, —C(O)—N(R^{IIIa})—C₁₋₆alkylene-phenyl, —C(O)—N(R^{IIIa})—C₁₋₆alkylene-4-7 membered heterocyclyl, —C(O)—N(R^{IIIa})—C₁₋₆alkylene-5-6 membered heteroaryl, —C═N(R^{IIIa})—C₁₋₆alkyl, —C═N(R^{IIIa})—N(R^{IIIa})—C₁₋₆alkyl, —S(O)₂—N(R^{IIIa})—C₁₋₆alkyl, and —S(O)₂—C₁₋₆alkyl;
   wherein C₁₋₆alkyl, C₂₋₆alkenyl, C₂₋₆alkynyl, C₃₋₆cycloalkyl, phenyl, 4-7 membered heterocyclyl, 5-6 membered heteroaryl, —C₁₋₆alkylene-C₃₋₈cycloalkyl, —C₁₋₆alkylene-phenyl, —C₁₋₆alkylene-4-7 membered heterocyclyl, —C₁₋₆alkylene-5-6 membered heteroaryl, —C(O)—C₁₋₆alkyl, —C(O)—O—C₁₋₆alkyl, —C(O)—C₁₋₆alkylene-C₃₋₈cycloalkyl, —C(O)—N(R^{IIIa})—C₁₋₆alkyl, —C(O)—N(R^{IIIa})—C₁₋₆alkylene-C₃₋₆cycloalkyl, —C(O)—N(R^{IIIa})—C₁₋₆alkylene-phenyl, —C(O)—N(R^{IIIa})—C₁₋₆alkylene-4-7 membered heterocyclyl, —C(O)—N(R^{IIIa})—C₁₋₆alkylene-5-6 membered heteroaryl, —C═N(R^{IIIa})—C₁₋₆alkyl and —S(O)₂—C₁₋₆alkyl may optionally be substituted on one or more available carbons by one, two, three or more substituents each independently selected from R^{IIIg}; and
   wherein if 4-7 membered heterocyclyl, 5-6 membered heteroaryl, —C₁₋₆alkylene-4-7 membered heterocyclyl, —C₁₋₆alkylene-5-6 membered heteroaryl, —C(O)—N(R^{IIIa})—C₁₋₆alkylene-4-7 membered heterocyclyl or —C(O)—N(R^{IIIa})—C₁₋₆alkylene-5-6 membered heteroaryl contains a substitutable ring nitrogen atom, that ring nitrogen atom may optionally be substituted by R^{IIIh}.
R^{III3} is selected from the group consisting of hydrogen, C₁₋₆alkyl, C₂₋₆alkenyl and C₂₋₆alkynyl;
R^{III4} is selected from the group consisting of hydrogen, halogen, C₁₋₆alkyl, C₂₋₆alkenyl and C₂₋₆alkynyl;
R^{III4′} is selected from the group consisting of hydrogen, halogen, C₁₋₆alkyl, C₂₋₆alkenyl and C₂₋₆alkynyl;
R^{III5} is selected from the group consisting of hydrogen, halogen and C₁₋₆alkyl;
R^{III6} is selected from the group consisting of hydrogen and deuterium;
R^{III7} is selected from the group consisting of hydrogen and deuterium;
R^{IIIg} is independently selected for each occurrence from the group consisting of hydrogen, halogen, hydroxyl, cyano, nitro, oxo, R^{IIIa}R^{IIIb}N—, R^{IIIA}R^{IIIb}N—C(O)—, R^{IIIa IIIb}N—SO_{w}—, R^{IIIa}R^{IIIb}N—C(O)—N(R^{IIIa})—, C₁₋₆alkyl, C₂₋₆alkenyl, C₂₋₆alkynyl, C₃₋₆cycloalkyl, C₃₋₆cycloalkyl-C₁₋₆alkylene-, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)_{w}—, C₁₋₆alkyl-N(R^{IIIa})—, C₁₋₆alkyl-N(R^{IIIa})—C(O)—, C₁₋₆alkyl-C(O)—N(R^{IIIa}), C₁₋₆alkyl-N(R^{IIIa})—C(O)—N(R^{IIIa})—, C₁₋₆alkyl-N(R^{IIIa})—SO_{w}—, C₃₋₆cycloalkyl-N(R^{IIIa})—SO_{w}—, C₁₋₆alkyl-SO_{w}—N(R^{IIIa})—, C₃₋₆cycloalkyl-SO_{w}—N(R^{IIIa})—, C₁₋₆alkoxy-C(O)—N(R^{IIIa})—, C₁₋₆alkyl-C(O)—N(R^{IIIa})—C₁₋₆alkyl-, C₁₋₆alkyl-N(R^{IIIa})—C(O)—C₁₋₆alkyl-, C₁₋₆alkoxy-C₁₋₆alkyl- and 5-6 membered heteroaryl;
   wherein C₁₋₆alkyl, C₂₋₆alkenyl, C₂₋₆alkynyl, C₃₋₆cycloalkyl, —C₁₋₆alkylene-C₃₋₆cycloalkyl, C₁₋₆alkoxy, C₃₋₆alkenyloxy, C₃₋₆alkynyloxy, C₃₋₆cycloalkoxy, C₁₋₆alkyl-C(O)—, C₁₋₆alkyl-O—C(O)—, C₁₋₆alkyl-C(O)—O—, C₁₋₆alkyl-S(O)—, C₁₋₆alkyl-N(R^{IIIa})—, C₁₋₆alkyl-N(R^{IIIa})—C(O)—, C₁₋₆alkyl-C(O)—N(R^{IIIa}), C₁₋₆alkyl-N(R^{IIIa})—C(O)—N(R^{IIIa})—, C₁₋₆alkyl-N(R^{IIIa})—SO_{w}—, C₃₋₆cycloalkyl-N(R^{IIIa})—SO_{w}—, C₁₋₆alkyl-SO_{w}—N(R^{IIIa})—, C₃₋₆cycloalkyl-SO_{w}—N(R^{IIIa})—, C₁₋₆alkoxy-C(O)—N(R^{IIIa})—, C₁₋₆alkyl-C(O)—N(R^{IIa})—C₁₋₆alkyl-, C₁₋₆alkyl-N(R^{IIIa})—C(O)—C₁₋₆alkyl-, C₁₋₆alkoxy-C₁₋₆alkyl- and 5-6 membered heteroaryl may optionally be substituted by one, two three or more substituents each independently selected from R^{IIIP};
R^{IIIh} is independently selected for each occurrence from the group consisting of C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{IIIa}R^{IIIb}N—C(O)—, R^{IIIa}R^{IIIb}N—SO₂— and —C₁₋₆alkylene-5-6 membered heteroaryl;
   wherein C₁₋₆alkyl, C₃₋₆alkenyl, C₃₋₆alkynyl, C₃₋₆cycloalkyl, C₁₋₆alkyl-S(O)₂—, C₃₋₆cycloalkyl-S(O)₂—, C₁₋₆alkyl-C(O)—, C₁₋₆alkoxy-C(O)—, R^{IIIa}R^{IIIb}N—C(O)—, R^{IIa}R^{IIIb}N—SO₂— and —C₁₋₆alkylene-5-6 membered heteroaryl may optionally be substituted by one, two three or more substituents each independently selected from R^{IIIP};
R^{IIIP} is independently selected for each occurrence from the group consisting of halogen, hydroxyl, cyano, C₁₋₆alkoxy, C₃₋₆cycloalkyl, R^{IIIa}R^{IIIb}N—, R^{IIIa}R^{IIIb}N-carbonyl-, R^{IIIa}R^{IIIb}N—SO₂—, and R^{IIIa}R^{IIIb}N-carbonyl-N(R^{IIIa})—;
R^{IIIa} and R^{IIIb} are independently selected, for each occurrence, from the group consisting of hydrogen and C₁₋₃alkyl;
   wherein C₁₋₃alkyl may optionally be substituted by one or more substituents each independently selected from the group consisting of halogen, cyano, oxo and hydroxyl:
or R^{IIIa} and R^{IIIb} together with the nitrogen to which they are attached form a 4-6 membered heterocyclyl;
   wherein 4-6 membered heterocyclyl may optionally be substituted by one or more substituents each independently selected from the group consisting of halogen, cyano, oxo and hydroxyl; and
w is 0, 1 or 2.

In some embodiments, R^{III1} is selected from the group consisting of hydrogen and oxo.

In some embodiments, R^{III6} is deuterium. In some embodiments, R^{III7} is deuterium. In some other embodiments, R^{III5}, R^{III6} and R^{III7} are each hydrogen.

In some embodiments, two or more of R^{III3}, R^{III4} and R^{III4′} are hydrogen. In some embodiments, R^{III3}, R^{III4} and R^{III4′} are each hydrogen.

In various embodiments, a compound disclosed herein is represented by:

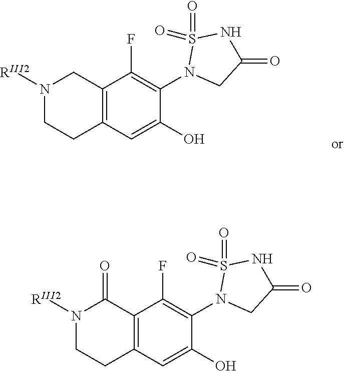

or a pharmaceutically acceptable salt thereof.

In various embodiments, R^{III2} is selected from the group consisting of hydrogen, C₁₋₆alkyl, —S(O)₂—NR^{IIIa}R^{IIIb}, and —C═N(R^{IIIa})—NR^{IIIa}R^{IIIb}, wherein R^{III2} may optionally be substituted by one, two, three or more substituents each independently selected from R^{IIIg}. For example, R^{III2} may be selected from the group consisting of hydrogen,

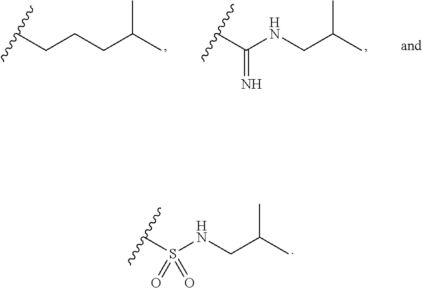

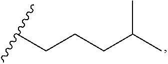 [SMILES: *CCCC(C)C]

In certain embodiments, when R^{III2} is hydrogen or R^{III1} is not hydrogen.

In some other embodiments, R^{III2} is selected from the group consisting of hydrogen, C₁₋₆alkyl and —C(O)—C₁₋₆alkyl, wherein R^{III2} may optionally be substituted by one, two, three or more substituents each independently selected from R^{IIIg}. For example R^{III2} may selected from the group consisting of hydrogen,

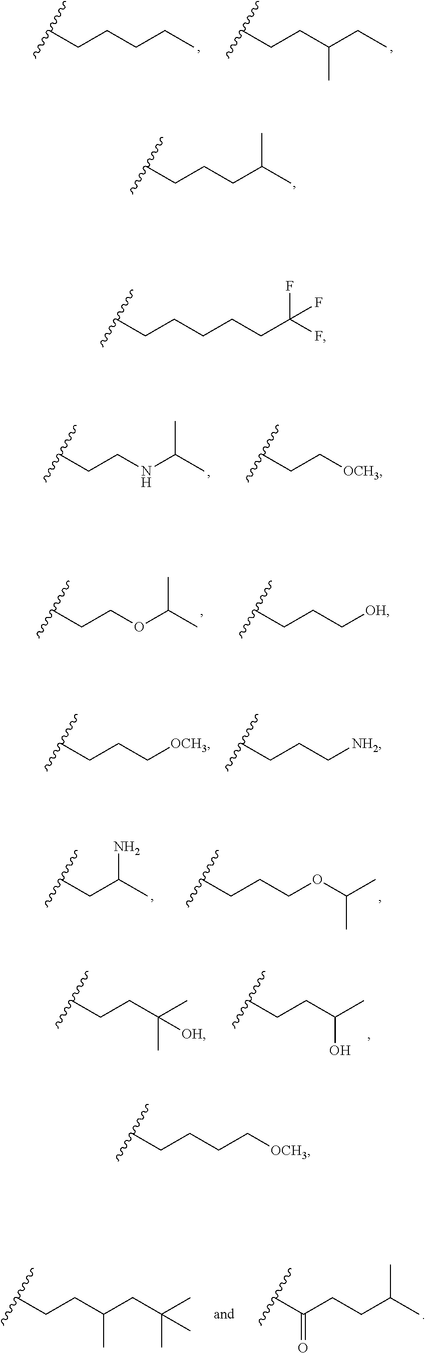

In some other embodiments, R^{III2} is 4-7 membered heterocyclyl, wherein 4-7 membered heterocyclyl may optionally be substituted on one or more available carbons by one, two, three or more substituents each independently selected from R^{IIIg}; and wherein if 4-7 membered heterocyclyl contains a substitutable ring nitrogen atom, that ring nitrogen atom may optionally be substituted by R^{IIIh}. For example, R^{III2} may be selected from the group consisting of

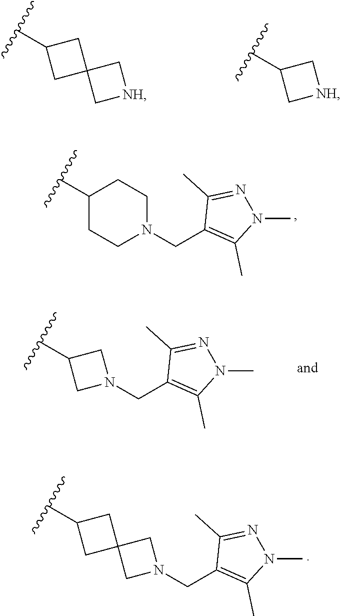 [SMILES: *C1CC2(CNC2)C1 ; *C1CNC1 ; *C1CCN(Cc2c(C)nn(C)c2C)CC1 ; *C1CN(Cc2c(C)nn(C)c2C)C1 ; *C1CC2(C1)CN(Cc1c(C)nn(C)c1C)C2]

In some other embodiments, R^{III2} is 5-6 membered heteroaryl, wherein 5-6 membered heteroaryl may optionally be substituted on one or more available carbons by one, two, three or more substituents each independently selected from R^{IIIg}; and wherein if 4-7 membered heterocyclyl contains a substitutable ring nitrogen atom, that ring nitrogen atom may optionally be substituted by R^{IIIh}. For example, R^{III2} may be represented by

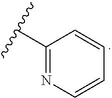 [SMILES: *c1ccccn1]

In some other embodiments, R^{III2} is —C₁₋₆alkylene-5-6 membered heteroaryl, wherein-C₁₋₆alkylene-5-6 membered heteroaryl may optionally be substituted on one or more available carbons by one, two, three or more substituents each independently selected from R^{IIIg}; and wherein if 4-7 membered heteroaryl contains a substitutable ring nitrogen atom, that ring nitrogen atom may optionally be substituted by R^{IIIh}. For example, R^{III2} may be selected from the group consisting of

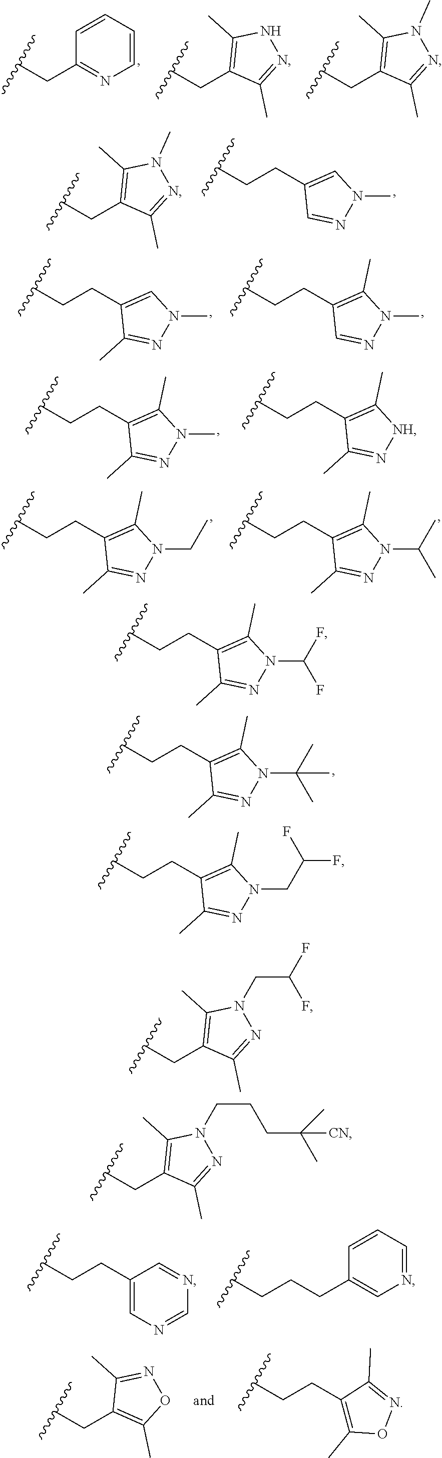

In some other embodiments, R^{III2} is —C₁₋₆alkylene-phenyl, wherein R^{III2} may optionally be substituted by one, two, three or more substituents each independently selected from R^{IIIg}. For example, R^{III2} may be selected from the group consisting of

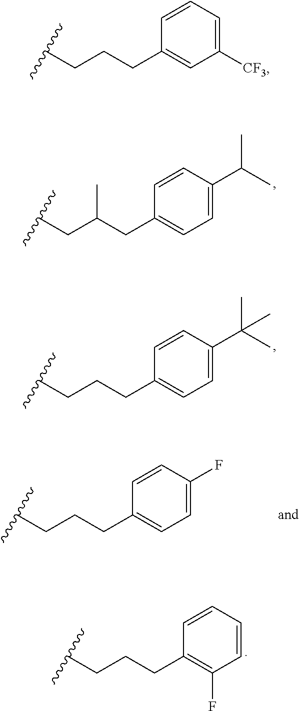 [SMILES: *CCCc1cccc(C(F)(F)F)c1 ; *CC(C)Cc1ccc(C(C)C)cc1 ; *CCCc1ccc(C(C)(C)C)cc1 ; *CCCc1ccc(F)cc1 ; *CCCc1ccccc1F]

In some other embodiments, R^{III2} is —C₁₋₆alkylene-4-7 membered heterocyclyl, wherein —C₁₋₆alkylene-4-7 membered heterocyclyl may optionally be substituted on one or more available carbons by one, two, three or more substituents each independently selected from R^{IIIg}; and wherein if —C₁₋₆alkylene-4-7 membered heterocyclyl contains a substitutable ring nitrogen atom, that ring nitrogen atom may optionally be substituted by R^{IIIh}; For example, R^{III2} may be selected from the group consisting of

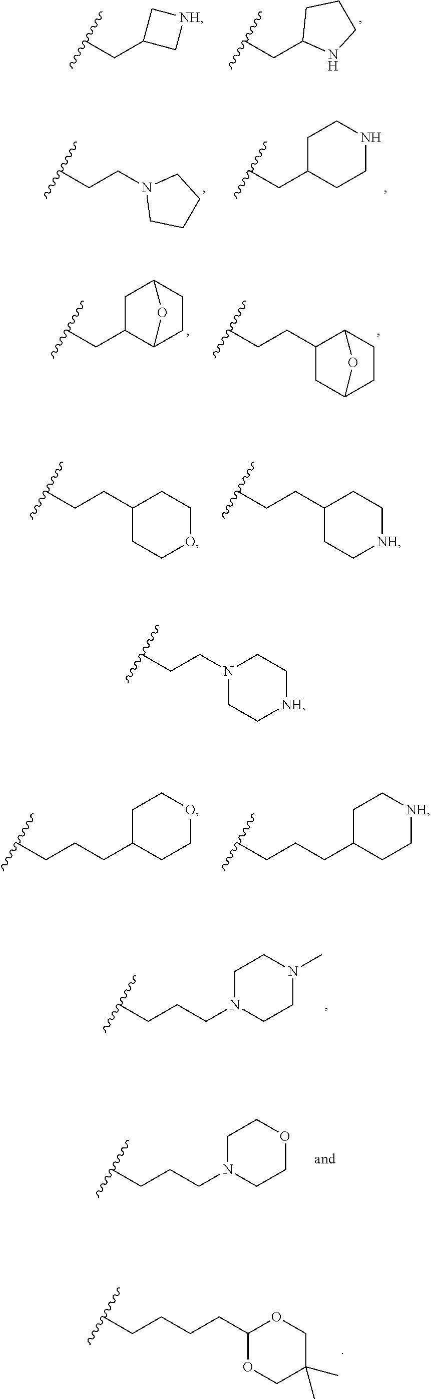

In some other embodiments, R^{III2} is —C(O)—O—C₁₋₆alkyl, wherein R^{III2} may optionally be substituted by one, two, three or more substituents each independently selected from R^{IIIg}. For example, R^{III2} is selected from the group consisting of

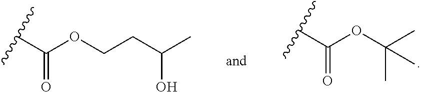

In some other embodiments, R^{III2} is —C(O)—N(R^{IIIa})—C₁₋₆alkyl, wherein R^{III2} may optionally be substituted by one, two, three or more substituents each independently selected from R^{IIIg}. For example, R^{III2} may be selected from the group consisting of

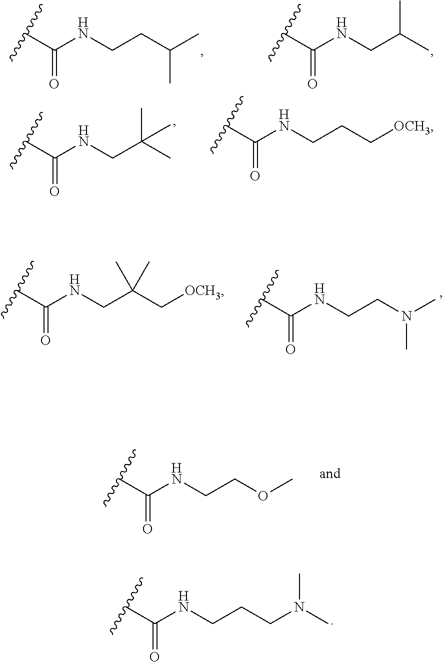

In some other embodiments, R^{III2} is —C₁₋₆alkylene-C₃₋₈cycloalkyl, wherein R^{III2} may optionally be substituted by one, two, three or more substituents each independently selected from R^{IIIg}. For example, R^{III2} may be selected from the group consisting of

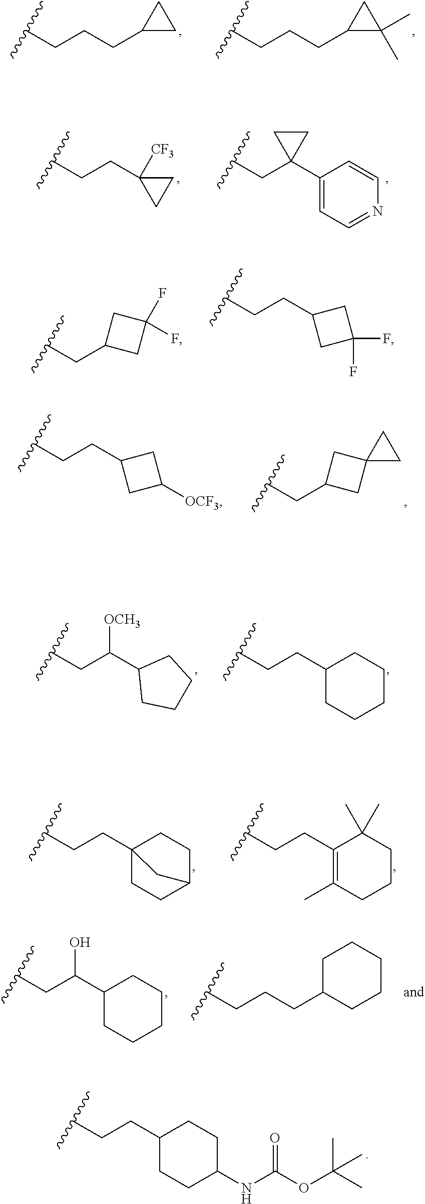

In some other embodiments, R^{III2} is —C(O)—C₁₋₆alkylene-C₃₋₈cycloalkyl, wherein R^{III2} may optionally be substituted by one, two, three or more substituents each independently selected from R^{IIIg}. For example, R^{III2} may be represented by

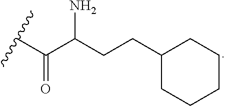 [SMILES: *C(=O)C(N)CCC1CCCCC1]

In some other embodiments, R^{III2} is —C═N(R^{IIIa})—C₁₋₆alkyl. For example, R^{III2} may be represented by

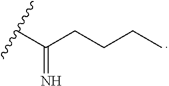 [SMILES: *C(=N)CCCC]

In some other embodiments, R^{III2} is selected from the group consisting of —C(O)—N(R^{IIIa})—C₁₋₆alkylene-C₃₋₆cycloalkyl, —C(O)—N(R^{IIIa})—C₁₋₆alkylene-phenyl, —C(O)—N(R^{IIIa})—C₁₋₆alkylene-4-7 membered heterocyclyl and —C(O)—N(R^{IIIa})—C₁₋₆alkylene-5-6 membered heteroaryl, wherein —C(O)—N(R^{IIIa})—C₁₋₆alkylene-C₃₋₆cycloalkyl, —C(O)—N(R^{IIIa})—C₁₋₆alkylene-phenyl, —C(O)—N(R^{IIIa})—C₁₋₆alkylene-4-7 membered heterocyclyl or —C(O)—N(R^{IIIa})—C₁₋₆alkylene-5-6 membered heteroaryl may optionally be substituted on one or more available carbons by one, two, three or more substituents each independently selected from R^{IIIg}; and wherein if-C(O)—N(R^{IIIa})—C₁₋₆alkylene-4-7 membered heterocyclyl or —C(O)—N(R^{IIIa})—C₁₋₆alkylene-5-6 membered heteroaryl contains a substitutable ring nitrogen atom, that ring nitrogen atom may optionally be substituted by R^{IIIh}. For example, R^{III2} may be selected from the group consisting of

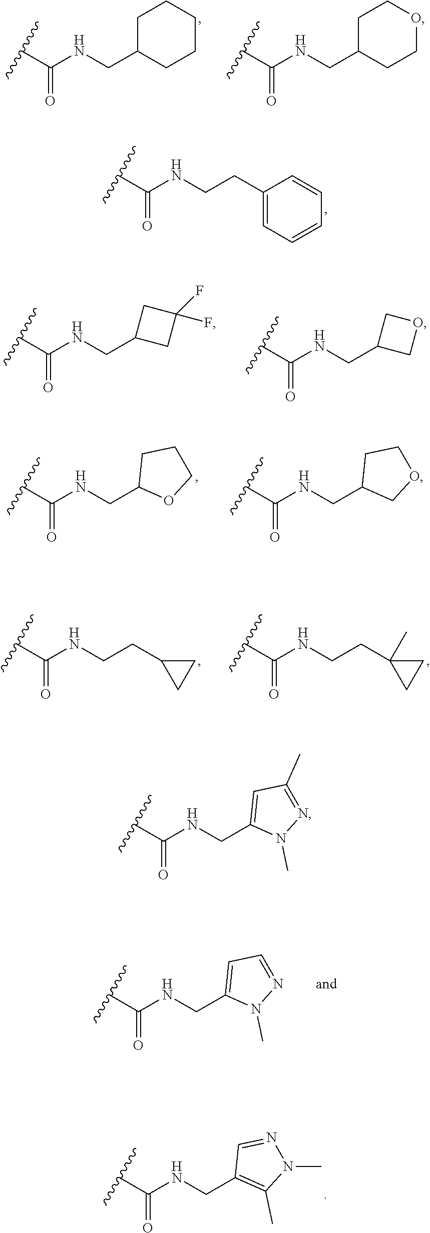

Further disclosed herein is a compound selected from the group consisting of:
5-[1-fluoro-3-hydroxy-7-(3-methylbutoxy)-5,6,7,8-tetrahydronaphthalen-2-yl]-1λ⁶,2,5-thiadiazolidine-1,1,3-trione;
5-{7-[(2-cyclopropylethyl)amino]-1-fluoro-3-hydroxy-5,6,7,8-tetrahydronaphthalen-2-yl}-1λ⁶,2,5-thiadiazolidine-1,1,3-trione;
5-{1-fluoro-3-hydroxy-7-[(3-methylbutyl)amino]-5,6,7,8-tetrahydronaphthalen-2-yl}-1λ⁶,2,5-thiadiazolidine-1,1,3-trione;
5-{7-[(cyclopropylmethyl)amino]-1-fluoro-3-hydroxy-5,6,7,8-tetrahydronaphthalen-2-yl}-1λ⁶,2,5-thiadiazolidine-1,1,3-trione;
5-[(7R)-1-fluoro-3-hydroxy-7-methoxy-5,6,7,8-tetrahydronaphthalen-2-yl]-1λ⁶,2,5-thiadiazolidine-1,1,3-trione;
5-[7-(2-cyclopropylethyl)-1-fluoro-3-hydroxy-5,6,7,8-tetrahydronaphthalen-2-yl]-1λ⁶,2,5-thiadiazolidine-1,1,3-trione;
5-[1-fluoro-3-hydroxy-7-(2-methoxyethoxy)-5,6,7,8-tetrahydronaphthalen-2-yl]-1λ⁶,2,5-thiadiazolidine-1,1,3-trione;
5-[7-(cyclopropylmethoxy)-1-fluoro-3-hydroxy-5,6,7,8-tetrahydronaphthalen-2-yl]-1λ⁶,2,5-thiadiazolidine-1,1,3-trione;
5-(1-fluoro-3-hydroxy-5,6,7,8-tetrahydronaphthalen-2-yl)-1λ⁶,2,5-thiadiazolidine-1,1,3-trione;
5-[(7S)-1-fluoro-3-hydroxy-7-methoxy-5,6,7,8-tetrahydronaphthalen-2-yl]-1λ⁶,2,5-thiadiazolidine-1,1,3-trione;
5-(1-fluoro-3-hydroxy-7-methoxy-5,6,7,8-tetrahydronaphthalen-2-yl)-1λ⁶,2,5-thiadiazolidine-1,1,3-trione;
5-(5-fluoro-7-hydroxy-3,4-dihydro-2H-1-benzopyran-6-yl)-1λ⁶,2,5-thiadiazolidine-1,1,3-trione;
5-(5-fluoro-7-hydroxy-2,2-dimethyl-3,4-dihydro-2H-1-benzopyran-6-yl)-1λ⁶,2,5-thiadiazolidine-1,1,3-trione;
5-(8-fluoro-6-hydroxy-2,2-dimethyl-3,4-dihydro-2H-1-benzopyran-7-yl)-1λ⁶,2,5-thiadiazolidine-1,1,3-trione;
5-(1,4-difluoro-3-hydroxy-7-methoxy-5,6,7,8-tetrahydronaphthalen-2-yl)-1λ⁶,2,5-thiadiazolidine-1,1,3-trione;
5-(7-{[2-(azetidin-1-yl)ethyl]amino}-1-fluoro-3-hydroxy-5,6,7,8-tetrahydronaphthalen-2-yl)-1λ⁶,2,5-thiadiazolidine-1,1,3-trione;
5-{1-fluoro-3-hydroxy-7-[(3,3,3-trifluoropropyl)amino]-5,6,7,8-tetrahydronaphthalen-2-yl}-1λ⁶,2,5-thiadiazolidine-1,1,3-trione;
5-{(7S)-1-fluoro-3-hydroxy-7-[(3-methylbutyl)amino]-5,6,7,8-tetrahydronaphthalen-2-yl}-1λ⁶,2,5-thiadiazolidine-1,1,3-trione;
5-{(7R)-1-fluoro-3-hydroxy-7-[(3-methylbutyl)amino]-5,6,7,8-tetrahydronaphthalen-2-yl}-1λ⁶,2,5-thiadiazolidine-1,1,3-trione;
5-{7-[(3,3-difluorocyclobutyl)methoxy]-1-fluoro-3-hydroxy-5,6,7,8-tetrahydronaphthalen-2-yl}-1λ⁶,2,5-thiadiazolidine-1,1,3-trione;
5-{1-fluoro-3-hydroxy-7-[(4,4,4-trifluorobutyl)amino]-5,6,7,8-tetrahydronaphthalen-2-yl}-1λ⁶,2,5-thiadiazolidine-1,1,3-trione;
5-{1-fluoro-3-hydroxy-7-[(4-methoxy-3,3-dimethylbutyl)amino]-5,6,7,8-tetrahydronaphthalen-2-yl}-1λ⁶,2,5-thiadiazolidine-1,1,3-trione;
5-{1-fluoro-3-hydroxy-7-[(3-methoxy-3-methylbutyl)amino]-5,6,7,8-tetrahydronaphthalen-2-yl}-1λ⁶,2,5-thiadiazolidine-1,1,3-trione;
5-{1-fluoro-3-hydroxy-7-[(4,4,4-trifluoro-3,3-dimethylbutyl)amino]-5,6,7,8-tetrahydronaphthalen-2-yl}-1λ⁶,2,5-thiadiazolidine-1,1,3-trione;
5-[1-fluoro-3-hydroxy-7-({2-[1-(trifluoromethyl)cyclopropyl]ethyl}amino)-5,6,7,8-tetrahydronaphthalen-2-yl]-1λ⁶,2,5-thiadiazolidine-1,1,3-trione;
5-{7-[(2,2-difluoro-2-phenylethyl)amino]-1-fluoro-3-hydroxy-5,6,7,8-tetrahydronaphthalen-2-yl}-1λ⁶,2,5-thiadiazolidine-1,1,3-trione;
5-{7-[(3-cyclopropyl-2,2-difluoropropyl)amino]-1-fluoro-3-hydroxy-5,6,7,8-tetrahydronaphthalen-2-yl}-1λ⁶2,5-thiadiazolidine-1,1,3-trione;
5-{1-fluoro-3-hydroxy-7-[(3-hydroxy-3-methylbutyl)amino]-5,6,7,8-tetrahydronaphthalen-2-yl}-1λ⁶,2,5-thiadiazolidine-1,1,3-trione;
5-{1-fluoro-3-hydroxy-7-[methyl (3-methylbutyl)amino]-5,6,7,8-tetrahydronaphthalen-2-yl}-1λ⁶,2,5-thiadiazolidine-1,1,3-trione;
5-{1-fluoro-3-hydroxy-7-[(4-methylpentyl)amino]-5,6,7,8-tetrahydronaphthalen-2-yl}-1λ⁶,2,5-thiadiazolidine-1,1,3-trione;
5-(1-fluoro-3-hydroxy-7-{[4,4,4-trifluoro-3-hydroxy-3-(trifluoromethyl)butyl]amino}-5,6,7,8-tetrahydronaphthalen-2-yl)-1λ⁶,2,5-thiadiazolidine-1,1,3-trione;
5-(1-fluoro-3-hydroxy-7-{[4,4,4-trifluoro-3-(trifluoromethyl)butyl]amino}-5,6,7,8-tetrahydronaphthalen-2-yl)-1λ⁶,2,5-thiadiazolidine-1,1,3-trione;
5-{7-[(2,2-difluoropropyl)amino]-1-fluoro-3-hydroxy-5,6,7,8-tetrahydronaphthalen-2-yl}-1λ⁶,2,5-thiadiazolidine-1,1,3-trione;
5-{(7R)-7-[(2-cyclopropylethyl)amino]-1-fluoro-3-hydroxy-5,6,7,8-tetrahydronaphthalen-2-yl}-1λ⁶,2,5-thiadiazolidine-1,1,3-trione;
5-{(7S)-7-[(2-cyclopropylethyl)amino]-1-fluoro-3-hydroxy-5,6,7,8-tetrahydronaphthalen-2-yl}-1λ⁶,2,5-thiadiazolidine-1,1,3-trione;
5-(1-fluoro-3-hydroxy-7-{[2-(pyridin-2-yl)ethyl]amino}-5,6,7,8-tetrahydronaphthalen-2-yl)-1λ⁶,2,5-thiadiazolidine-1,1,3-trione;
5-{(7RS)-1-fluoro-3-hydroxy-7-[(3RS)-pyrrolidin-3-yl]-5,6,7,8-tetrahydronaphthalen-2-yl}-1λ⁶,2,5-thiadiazolidine-1,1,3-trione;
5-{(7RS)-7-[(3RS)-1-(cyclopropanesulfonyl) pyrrolidin-3-yl]-1-fluoro-3-hydroxy-5,6,7,8-tetrahydronaphthalen-2-yl}-1λ⁶,2,5-thiadiazolidine-1,1,3-trione;
5-{(7RS)-1-fluoro-3-hydroxy-7-[(3SR)-pyrrolidin-3-yl]-5,6,7,8-tetrahydronaphthalen-2-yl}-1λ⁶,2,5-thiadiazolidine-1,1,3-trione;
5-{(7RS)-7-[(3SR)-1-(cyclopropanesulfonyl) pyrrolidin-3-yl]-1-fluoro-3-hydroxy-5,6,7,8-tetrahydronaphthalen-2-yl}-1λ⁶,2,5-thiadiazolidine-1,1,3-trione;
5-{7-[1-(cyclopropylmethyl) pyrrolidin-3-yl]-1-fluoro-3-hydroxy-5,6,7,8-tetrahydronaphthalen-2-yl}-1λ⁶,2,5-thiadiazolidine-1,1,3-trione;
5-(1-fluoro-3-hydroxy-7-{[2-(1H-pyrazol-1-yl)ethyl]amino}-5,6,7,8-tetrahydronaphthalen-2-yl)-1λ⁶,2,5-thiadiazolidine-1,1,3-trione;
5-[1-fluoro-3-hydroxy-7-(4,4,4-trifluorobutoxy)-5,6,7,8-tetrahydronaphthalen-2-yl]-1λ⁶,2,5-thiadiazolidine-1,1,3-trione;
5-(8-fluoro-6-hydroxy-1,2,3,4-tetrahydroisoquinolin-7-yl)-1λ⁶,2,5-thiadiazolidine-1,1,3-trione;
5-[8-fluoro-6-hydroxy-2-(4-methylpentanoyl)-1,2,3,4-tetrahydroisoquinolin-7-yl]-1λ⁶,2,5-thiadiazolidine-1,1,3-trione;
5-[8-fluoro-6-hydroxy-2-(4-methylpentyl)-1,2,3,4-tetrahydroisoquinolin-7-yl]-1λ⁶,2,5-thiadiazolidine-1,1,3-trione;
5-{(7R)-1-fluoro-3-hydroxy-7-[(4,4,4-trifluorobutyl)amino]-5,6,7,8-tetrahydronaphthalen-2-yl}-1λ⁶,2,5-thiadiazolidine-1,1,3-trione;
5-{(7S)-1-fluoro-3-hydroxy-7-[(4,4,4-trifluorobutyl)amino]-5,6,7,8-tetrahydronaphthalen-2-yl}-1λ⁶,2,5-thiadiazolidine-1,1,3-trione;
5-[(7R)-1-fluoro-3-hydroxy-7-({2-[1-(trifluoromethyl)cyclopropyl]ethyl}amino)-5,6,7,8-tetrahydronaphthalen-2-yl]-1λ⁶,2,5-thiadiazolidine-1,1,3-trione;
8-fluoro-6-hydroxy-7-(1,1,4-trioxo-1λ⁶,2,5-thiadiazolidin-2-yl)-1,2,3,4-tetrahydronaphthalen-2-yl phenylcarbamate;
4-{[8-fluoro-6-hydroxy-7-(1,1,4-trioxo-1λ⁶,2,5-thiadiazolidin-2-yl)-1,2,3,4-tetrahydronaphthalen-2-yl]amino}-2,2-dimethylbutanenitrile;
5-{1-fluoro-3-hydroxy-7-[(4,4,4-trifluoro-3-hydroxybutyl)amino]-5,6,7,8-tetrahydronaphthalen-2-yl}-1λ⁶,2,5-thiadiazolidine-1,1,3-trione;
5-{1-fluoro-3-hydroxy-7-[(4,4,4-trifluoro-3-methoxybutyl)amino]-5,6,7,8-tetrahydronaphthalen-2-yl}-1λ⁶,2,5-thiadiazolidine-1,1,3-trione;
5-[8-fluoro-6-hydroxy-2-(5,5,5-trifluoropentyl)-1,2,3,4-tetrahydroisoquinolin-7-yl]-1λ⁶,2,5-thiadiazolidine-1,1,3-trione;
5-(1-fluoro-3-hydroxy-7-{(3-methylbutyl)[(pyridin-2-yl)methyl]amino}-5,6,7,8-tetrahydronaphthalen-2-yl)-1λ⁶,2,5-thiadiazolidine-1,1,3-trione;
5-(1-fluoro-3-hydroxy-7-{[(pyridin-2-yl)methyl]amino}-5,6,7,8-tetrahydronaphthalen-2-yl)-1λ⁶,2,5-thiadiazolidine-1,1,3-trione;
5-{1-fluoro-3-hydroxy-7-[(4,4,4-trifluoro-2-hydroxybutyl)amino]-5,6,7,8-tetrahydronaphthalen-2-yl}-1λ⁶,2,5-thiadiazolidine-1,1,3-trione;
5-(7-{[2-(difluoromethoxy)ethyl]amino}-1-fluoro-3-hydroxy-5,6,7,8-tetrahydronaphthalen-2-yl)-1λ⁶,2,5-thiadiazolidine-1,1,3-trione;
5-(8-fluoro-6-hydroxy-2-pentanimidoyl-1,2,3,4-tetrahydroisoquinolin-7-yl)-1λ⁶,2,5-thiadiazolidine-1,1,3-trione;
5-[2-(3-cyclopropylpropyl)-8-fluoro-6-hydroxy-1,2,3,4-tetrahydroisoquinolin-7-yl]-1λ⁶,2,5-thiadiazolidine-1,1,3-trione;
5-[2-(2-azaspiro[3.3]heptan-6-yl)-8-fluoro-6-hydroxy-1,2,3,4-tetrahydroisoquinolin-7-yl]-1λ⁶,2,5-thiadiazolidine-1,1,3-trione;
5-[8-fluoro-6-hydroxy-2-(6,6,6-trifluorohexyl)-1,2,3,4-tetrahydroisoquinolin-7-yl]-1λ⁶,2,5-thiadiazolidine-1,1,3-trione;
5-{2-[(3,3-difluorocyclobutyl)methyl]-8-fluoro-6-hydroxy-1,2,3,4-tetrahydroisoquinolin-7-yl}-1λ⁶,2,5-thiadiazolidine-1,1,3-trione;
5-[2-(azetidin-3-yl)-8-fluoro-6-hydroxy-1,2,3,4-tetrahydroisoquinolin-7-yl]-1λ⁶,2,5-thiadiazolidine-1,1,3-trione;
5-(8-fluoro-6-hydroxy-2-{2-[(propan-2-yl)amino]ethyl}-1,2,3,4-tetrahydroisoquinolin-7-yl)-1λ⁶,2,5-thiadiazolidine-1,1,3-trione;
5-{2-[(azetidin-3-yl)methyl]-8-fluoro-6-hydroxy-1,2,3,4-tetrahydroisoquinolin-7-yl}-1λ⁶,2,5-thiadiazolidine-1,1,3-trione;
5-{2-[(azetidin-3-yl)methyl]-8-fluoro-6-hydroxy-1,2,3,4-tetrahydroisoquinolin-7-yl}-1λ⁶,2,5-thiadiazolidine-1,1,3-trione;
5-(1-fluoro-3-hydroxy-7-{(3-methylbutyl)[2-(pyridin-2-yl)ethyl]amino}-5,6,7,8-tetrahydronaphthalen-2-yl)-1λ⁶,2,5-thiadiazolidine-1,1,3-trione;
5-{8-fluoro-6-hydroxy-2-[(spiro[2.3]hexan-5-yl)methyl]-1,2,3,4-tetrahydroisoquinolin-7-yl}-1λ⁶,2,5-thiadiazolidine-1,1,3-trione;
5-(1-fluoro-3-hydroxy-7-{[2-(trifluoromethoxy)ethyl]amino}-5,6,7,8-tetrahydronaphthalen-2-yl)-1λ⁶,2,5-thiadiazolidine-1,1,3-trione;
5-[8-fluoro-6-hydroxy-2-(3-hydroxy-3-methylbutyl)-1,2,3,4-tetrahydroisoquinolin-7-yl]-1λ⁶,2,5-thiadiazolidine-1,1,3-trione;
3-hydroxybutyl 8-fluoro-6-hydroxy-7-(1,1,4-trioxo-1λ⁶,2,5-thiadiazolidin-2-yl)-3,4-dihydroisoquinoline-2(1H)-carboxylate;
5-{1-fluoro-3-hydroxy-7-[3-(propan-2-yl) pyrrolidin-1-yl]-5,6,7,8-tetrahydronaphthalen-2-yl}-1λ⁶,2,5-thiadiazolidine-1,1,3-trione;
5-{7-[(2-cyclohexylethyl)amino]-1-fluoro-3-hydroxy-5,6,7,8-tetrahydronaphthalen-2-yl}-1λ⁶,2,5-thiadiazolidine-1,1,3-trione;
5-{7-[(3,3-dimethylbutyl)amino]-1-fluoro-3-hydroxy-5,6,7,8-tetrahydronaphthalen-2-yl}-1λ⁶,2,5-thiadiazolidine-1,1,3-trione;
5-[7-(butylamino)-1-fluoro-3-hydroxy-5,6,7,8-tetrahydronaphthalen-2-yl]-1λ⁶,2,5-thiadiazolidine-1,1,3-trione;
5-{(7S)-1-fluoro-3-hydroxy-7-[(4,4,4-trifluoro-3,3-dimethylbutyl)amino]-5,6,7,8-tetrahydronaphthalen-2-yl}-1λ⁶,2,5-thiadiazolidine-1,1,3-trione;
5-{(7R)-1-fluoro-3-hydroxy-7-[(4,4,4-trifluoro-3,3-dimethylbutyl)amino]-5,6,7,8-tetrahydronaphthalen-2-yl}-1λ⁶,2,5-thiadiazolidine-1,1,3-trione;
5-{7-[(2-cyclopentylethyl)amino]-1-fluoro-3-hydroxy-5,6,7,8-tetrahydronaphthalen-2-yl}-1λ⁶,2,5-thiadiazolidine-1,1,3-trione;
5-[2-(2-cyclohexylethyl)-8-fluoro-6-hydroxy-1,2,3,4-tetrahydroisoquinolin-7-yl]-1λ⁶,2,5-thiadiazolidine-1,1,3-trione;
5-{1-fluoro-3-hydroxy-7-[(2-hydroxyethyl)amino]-5,6,7,8-tetrahydronaphthalen-2-yl}-1λ⁶,2,5-thiadiazolidine-1,1,3-trione;
5-{1-fluoro-3-hydroxy-7-[2-(propan-2-yl) morpholin-4-yl]-5,6,7,8-tetrahydronaphthalen-2-yl}-1λ⁶,2,5-thiadiazolidine-1,1,3-trione;
5-{1-fluoro-3-hydroxy-7-[(2R)-2-(propan-2-yl) morpholin-4-yl]-5,6,7,8-tetrahydronaphthalen-2-yl}-1λ⁶,2,5-thiadiazolidine-1,1,3-trione;
5-{8-fluoro-6-hydroxy-2-[(pyrrolidin-2-yl)methyl]-1,2,3,4-tetrahydroisoquinolin-7-yl}-1λ⁶,2,5-thiadiazolidine-1,1,3-trione;
5-{8-fluoro-6-hydroxy-2-[(pyridin-2-yl)methyl]-1,2,3,4-tetrahydroisoquinolin-7-yl}-1λ⁶,2,5-thiadiazolidine-1,1,3-trione;
5-{7-[(2-cyclobutylethyl)amino]-1-fluoro-3-hydroxy-5,6,7,8-tetrahydronaphthalen-2-yl}-1λ⁶,2,5-thiadiazolidine-1,1,3-trione;
5-[1-fluoro-3-hydroxy-7-({2-[(propan-2-yl)oxy]ethyl}amino)-5,6,7,8-tetrahydronaphthalen-2-yl]-1λ⁶,2,5-thiadiazolidine-1,1,3-trione;
5-{1-fluoro-3-hydroxy-7-[(2-hydroxy-3-methylbutyl)amino]-5,6,7,8-tetrahydronaphthalen-2-yl}-1λ⁶,2,5-thiadiazolidine-1,1,3-trione;
5-{7-[(2-cyclopropyl-2-hydroxyethyl)amino]-1-fluoro-3-hydroxy-5,6,7,8-tetrahydronaphthalen-2-yl}-1λ⁶,2,5-thiadiazolidine-1,1,3-trione;
5-(1-fluoro-3-hydroxy-7-{[3-(trimethylsilyl)propyl]amino}-5,6,7,8-tetrahydronaphthalen-2-yl)-1λ⁶,2,5-thiadiazolidine-1,1,3-trione;
5-[1-fluoro-3-hydroxy-7-({3-[hydroxy(dimethyl)silyl]propyl}amino)-5,6,7,8-tetrahydronaphthalen-2-yl]-1λ⁶,2,5-thiadiazolidine-1,1,3-trione;
5-[8-fluoro-6-hydroxy-2-(pyridin-2-yl)-1,2,3,4-tetrahydroisoquinolin-7-yl]-1λ⁶,2,5-thiadiazolidine-1,1,3-trione;
5-{2-[2-(3,3-difluorocyclobutyl)ethyl]-8-fluoro-6-hydroxy-1,2,3,4-tetrahydroisoquinolin-7-yl}-1λ⁶,2,5-thiadiazolidine-1,1,3-trione;
5-{8-fluoro-6-hydroxy-2-[2-(pyrrolidin-1-yl)ethyl]-1,2,3,4-tetrahydroisoquinolin-7-yl}-1λ⁶,2,5-thiadiazolidine-1,1,3-trione;
5-{2-[2-(3,5-dimethyl-1H-pyrazol-4-yl)ethyl]-8-fluoro-6-hydroxy-1,2,3,4-tetrahydroisoquinolin-7-yl}-1λ⁶,2,5-thiadiazolidine-1,1,3-trione;
N-[8-fluoro-6-hydroxy-7-(1,1,4-trioxo-1λ⁶,2,5-thiadiazolidin-2-yl)-1,2,3,4-tetrahydronaphthalen-2-yl]-3-methylbutanimidamide;
5-{8-fluoro-6-hydroxy-2-[3-(oxan-4-yl)propyl]-1,2,3,4-tetrahydroisoquinolin-7-yl}-1λ⁶,2,5-thiadiazolidine-1,1,3-trione;
5-[1-fluoro-3-hydroxy-7-(3-hydroxy-3-methylbutoxy)-5,6,7,8-tetrahydronaphthalen-2-yl]-1λ⁶,2,5-thiadiazolidine-1,1,3-trione;
5-[(7R)-7-amino-1-fluoro-3-hydroxy-5,6,7,8-tetrahydronaphthalen-2-yl]-1λ⁶,2,5-thiadiazolidine-1,1,3-trione;
5-{(7R)-7-[(4,4-difluorobutyl)amino]-1-fluoro-3-hydroxy-5,6,7,8-tetrahydronaphthalen-2-yl}-1λ⁶,2,5-thiadiazolidine-1,1,3-trione;
5-{(7R)-7-[(2-cyclopentylethyl)amino]-1-fluoro-3-hydroxy-5,6,7,8-tetrahydronaphthalen-2-yl}-1λ⁶,2,5-thiadiazolidine-1,1,3-trione;
5-{(7R)-7-[(2-cyclobutylethyl)amino]-1-fluoro-3-hydroxy-5,6,7,8-tetrahydronaphthalen-2-yl}-1λ⁶,2,5-thiadiazolidine-1,1,3-trione;
5-[(7R)-7-{[2-(3,3-difluorocyclobutyl)ethyl]amino}-1-fluoro-3-hydroxy-5,6,7,8-tetrahydronaphthalen-2-yl]-1λ⁶,2,5-thiadiazolidine-1,1,3-trione;
5-{(7R)-1-fluoro-3-hydroxy-7-[(3-methylpentyl)amino]-5,6,7,8-tetrahydronaphthalen-2-yl}-1λ⁶,2,5-thiadiazolidine-1,1,3-trione;
5-{(7R)-7-[(3-ethylpentyl)amino]-1-fluoro-3-hydroxy-5,6,7,8-tetrahydronaphthalen-2-yl}-1λ⁶,2,5-thiadiazolidine-1,1,3-trione;
8-fluoro-6-hydroxy-N-(3-methylbutyl)-7-(1,1,4-trioxo-1λ⁶,2,5-thiadiazolidin-2-yl)-3,4-dihydroisoquinoline-2(1H)-carboxamide;
8-fluoro-6-hydroxy-N-(2-methylpropyl)-7-(1,1,4-trioxo-1λ⁶,2,5-thiadiazolidin-2-yl)-3,4-dihydroisoquinoline-2(1H)-carboxamide;
5-{8-fluoro-6-hydroxy-2-[3-(pyridin-3-yl)propyl]-1,2,3,4-tetrahydroisoquinolin-7-yl}-1λ⁶,2,5-thiadiazolidine-1,1,3-trione;
5-{2-[(3,5-dimethyl-1,2-oxazol-4-yl)methyl]-8-fluoro-6-hydroxy-1,2,3,4-tetrahydroisoquinolin-7-yl}-1λ⁶,2,5-thiadiazolidine-1,1,3-trione;
5-{8-fluoro-6-hydroxy-2-[2-(1,3,5-trimethyl-1H-pyrazol-4-yl)ethyl]-1,2,3,4-tetrahydroisoquinolin-7-yl}-1λ⁶,2,5-thiadiazolidine-1,1,3-trione;
5-{8-fluoro-6-hydroxy-2-[2-(pyrimidin-5-yl)ethyl]-1,2,3,4-tetrahydroisoquinolin-7-yl}-1λ⁶,2,5-thiadiazolidine-1,1,3-trione;
5-{2-[2-(3,5-dimethyl-1,2-oxazol-4-yl)ethyl]-8-fluoro-6-hydroxy-1,2,3,4-tetrahydroisoquinolin-7-yl}-1λ⁶,2,5-thiadiazolidine-1,1,3-trione;
5-{2-[(3,5-dimethyl-1H-pyrazol-4-yl)methyl]-8-fluoro-6-hydroxy-1,2,3,4-tetrahydroisoquinolin-7-yl}-1λ⁶,2,5-thiadiazolidine-1,1,3-trione;
5-{8-fluoro-6-hydroxy-2-[(1,3,5-trimethyl-1H-pyrazol-4-yl)methyl]-1,2,3,4-tetrahydroisoquinolin-7-yl}-1λ⁶,2,5-thiadiazolidine-1,1,3-trione;
5-{8-fluoro-6-hydroxy-2-[2-(oxan-4-yl)ethyl]-1,2,3,4-tetrahydroisoquinolin-7-yl}-1λ⁶,2,5-thiadiazolidine-1,1,3-trione;
5-[2-(2-cyclohexyl-2-hydroxyethyl)-8-fluoro-6-hydroxy-1,2,3,4-tetrahydroisoquinolin-7-yl]-1λ⁶,2,5-thiadiazolidine-1,1,3-trione;
5-[8-fluoro-6-hydroxy-2-(2-methoxyethyl)-1,2,3,4-tetrahydroisoquinolin-7-yl]-1λ⁶,2,5-thiadiazolidine-1,1,3-trione;
5-[8-fluoro-6-hydroxy-2-(3-methoxypropyl)-1,2,3,4-tetrahydroisoquinolin-7-yl]-1λ⁶,2,5-thiadiazolidine-1,1,3-trione;
5-[2-(3-aminopropyl)-8-fluoro-6-hydroxy-1,2,3,4-tetrahydroisoquinolin-7-yl]-1λ⁶,2,5-thiadiazolidine-1,1,3-trione;
5-{8-fluoro-6-hydroxy-2-[3-(piperidin-4-yl)propyl]-1,2,3,4-tetrahydroisoquinolin-7-yl}-1λ⁶,2,5-thiadiazolidine-1,1,3-trione;
5-[8-fluoro-6-hydroxy-2-(3-methylbutyl)-1,2,3,4-tetrahydroisoquinolin-7-yl]-1λ⁶,2,5-thiadiazolidine-1,1,3-trione;
tert-butyl 8-fluoro-6-hydroxy-7-(1,1,4-trioxo-1λ⁶,2,5-thiadiazolidin-2-yl)-3,4-dihydroisoquinoline-2(1H)-carboxylate;
5-{8-fluoro-6-hydroxy-2-[(7-oxabicyclo[2.2.1]heptan-2-yl)methyl]-1,2,3,4-tetrahydroisoquinolin-7-yl}-1λ⁶,2,5-thiadiazolidine-1,1,3-trione;
5-{7-[(3,3-difluoropropyl)amino]-1-fluoro-3-hydroxy-5,6,7,8-tetrahydronaphthalen-2-yl}-1λ⁶,2,5-thiadiazolidine-1,1,3-trione;
5-(8-fluoro-6-hydroxy-2-{1-[(1,3,5-trimethyl-1H-pyrazol-4-yl)methyl]azetidin-3-yl}-1,2,3,4-tetrahydroisoquinolin-7-yl)-1λ⁶,2,5-thiadiazolidine-1,1,3-trione;
5-{8-fluoro-6-hydroxy-2-[2-(7-oxabicyclo[2.2.1]heptan-2-yl)ethyl]-1,2,3,4-tetrahydroisoquinolin-7-yl}-1λ⁶,2,5-thiadiazolidine-1,1,3-trione;
5-(2-{2-[1-(2,2-difluoroethyl)-3,5-dimethyl-1H-pyrazol-4-yl]ethyl}-4,4,8-trifluoro-6-hydroxy-1,2,3,4-tetrahydroisoquinolin-7-yl)-1λ⁶,2,5-thiadiazolidine-1,1,3-trione;
5-(4-{[8-fluoro-6-hydroxy-7-(1,1,4-trioxo-1λ⁶,2,5-thiadiazolidin-2-yl)-3,4-dihydroisoquinolin-2(1H)-yl]methyl}-3,5-dimethyl-1H-pyrazol-1-yl)-2,2-dimethylpentanenitrile;
5-{8-fluoro-6-hydroxy-2-[(piperidin-4-yl)methyl]-1,2,3,4-tetrahydroisoquinolin-7-yl}-1λ⁶,2,5-thiadiazolidine-1,1,3-trione;
5-{8-fluoro-6-hydroxy-2-[3-(morpholin-4-yl)propyl]-1,2,3,4-tetrahydroisoquinolin-7-yl}-1λ⁶,2,5-thiadiazolidine-1,1,3-trione;
5-{8-fluoro-6-hydroxy-2-[2-(piperidin-4-yl)ethyl]-1,2,3,4-tetrahydroisoquinolin-7-yl}-1λ⁶,2,5-thiadiazolidine-1,1,3-trione;
5-(8-fluoro-6-hydroxy-2-{2-[(1s,3r)-3-(trifluoromethoxy)cyclobutyl]ethyl}-1,2,3,4-tetrahydroisoquinolin-7-yl)-1λ⁶,2,5-thiadiazolidine-1,1,3-trione;
5-{8-fluoro-6-hydroxy-2-[3-(4-methylpiperazin-1-yl)propyl]-1,2,3,4-tetrahydroisoquinolin-7-yl}-1λ⁶,2,5-thiadiazolidine-1,1,3-trione;
5-(8-fluoro-6-hydroxy-2-{2-[(propan-2-yl)oxy]ethyl}-1,2,3,4-tetrahydroisoquinolin-7-yl)-1λ⁶,2,5-thiadiazolidine-1,1,3-trione;
5-[1-fluoro-3-hydroxy-7-({2-[(1s,3r)-3-(trifluoromethoxy)cyclobutyl]ethyl}amino)-5,6,7,8-tetrahydronaphthalen-2-yl]-1λ⁶,2,5-thiadiazolidine-1,1,3-trione;
5-(1-fluoro-3-hydroxy-7-{[(1s,3s)-3-(trifluoromethoxy)cyclobutyl]amino}-5,6,7,8-tetrahydronaphthalen-2-yl)-1λ⁶,2,5-thiadiazolidine-1,1,3-trione;
5-(8-fluoro-6-hydroxy-2-{1-[(1,3,5-trimethyl-1H-pyrazol-4-yl)methyl]piperidin-4-yl}-1,2,3,4-tetrahydroisoquinolin-7-yl)-1λ⁶,2,5-thiadiazolidine-1,1,3-trione;
5-(8-fluoro-6-hydroxy-2-{2-[(1,3,5-trimethyl-1H-pyrazol-4-yl)methyl]-2-azaspiro[3.3]heptan-6-yl}-1,2,3,4-tetrahydroisoquinolin-7-yl)-1λ⁶,2,5-thiadiazolidine-1,1,3-trione;
5-(2-{2-[1-(difluoromethyl)-3,5-dimethyl-1H-pyrazol-4-yl]ethyl}-8-fluoro-6-hydroxy-1,2,3,4-tetrahydroisoquinolin-7-yl)-1λ⁶,2,5-thiadiazolidine-1,1,3-trione;
5-{2-[2-(bicyclo[2.2.1]heptan-1-yl)ethyl]-8-fluoro-6-hydroxy-1,2,3,4-tetrahydroisoquinolin-7-yl}-1λ⁶,2,5-thiadiazolidine-1,1,3-trione;
5-[7-amino-1-fluoro-3-hydroxy-7-(prop-2-en-1-yl)-5,6,7,8-tetrahydronaphthalen-2-yl]-1λ⁶,2,5-thiadiazolidine-1,1,3-trione;
N′-[8-fluoro-6-hydroxy-2-propyl-7-(1,1,4-trioxo-1λ⁶,2,5-thiadiazolidin-2-yl)-1,2,3,4-tetrahydronaphthalen-2-yl]benzohydrazide;
5-[8-fluoro-6-hydroxy-2-(3-hydroxybutyl)-1,2,3,4-tetrahydroisoquinolin-7-yl]-1λ⁶,2,5-thiadiazolidine-1,1,3-trione;
5-(8-fluoro-6-hydroxy-2-{2-[1-(trifluoromethyl)cyclopropyl]ethyl}-1,2,3,4-tetrahydroisoquinolin-7-yl)-1λ⁶,2,5-thiadiazolidine-1,1,3-trione;
5-[8-fluoro-6-hydroxy-2-(3-hydroxypropyl)-1,2,3,4-tetrahydroisoquinolin-7-yl]-1λ⁶,2,5-thiadiazolidine-1,1,3-trione;
5-{2-[(2S)-2-aminopropyl]-8-fluoro-6-hydroxy-1,2,3,4-tetrahydroisoquinolin-7-yl}-1λ⁶,2,5-thiadiazolidine-1,1,3-trione;
5-{2-[(2R)-2-aminopropyl]-8-fluoro-6-hydroxy-1,2,3,4-tetrahydroisoquinolin-7-yl}-1λ⁶,2,5-thiadiazolidine-1,1,3-trione;
5-{8-fluoro-6-hydroxy-2-[2-(piperazin-1-yl)ethyl]-1,2,3,4-tetrahydroisoquinolin-7-yl}-1λ⁶,2,5-thiadiazolidine-1,1,3-trione;
5-(8-fluoro-6-hydroxy-2-{[rac-(1R,2R)-2-(pyridin-4-yl)cyclopropyl]methyl}-1,2,3,4-tetrahydroisoquinolin-7-yl)-1λ⁶,2,5-thiadiazolidine-1,1,3-trione;
5-[2-(2-cyclopentyl-2-methoxyethyl)-8-fluoro-6-hydroxy-1,2,3,4-tetrahydroisoquinolin-7-yl]-1λ⁶,2,5-thiadiazolidine-1,1,3-trione;
5-{2-[(2R)-2-amino-4-cyclohexylbutanoyl]-8-fluoro-6-hydroxy-1,2,3,4-tetrahydroisoquinolin-7-yl}-1λ⁶,2,5-thiadiazolidine-1,1,3-trione;
5-(8-fluoro-6-hydroxy-2-{3-[(propan-2-yl)oxy]propyl}-1,2,3,4-tetrahydroisoquinolin-7-yl)-1λ⁶,2,5-thiadiazolidine-1,1,3-trione;
5-{8-fluoro-6-hydroxy-2-[2-(1-methyl-1H-pyrazol-4-yl)ethyl]-1,2,3,4-tetrahydroisoquinolin-7-yl}-1λ⁶,2,5-thiadiazolidine-1,1,3-trione;
5-(2-{2-[1-(2,2-difluoroethyl)-3,5-dimethyl-1H-pyrazol-4-yl]ethyl}-8-fluoro-6-hydroxy-1,2,3,4-tetrahydroisoquinolin-7-yl)-1λ⁶,2,5-thiadiazolidine-1,1,3-trione;
5-[7-amino-1-fluoro-3-hydroxy-7-(4-methylpentyl)-5,6,7,8-tetrahydronaphthalen-2-yl]-1λ⁶,2,5-thiadiazolidine-1,1,3-trione;
5-(7-amino-1-fluoro-3-hydroxy-7-propyl-5,6,7,8-tetrahydronaphthalen-2-yl)-1λ⁶,2,5-thiadiazolidine-1,1,3-trione;
5-{2-[2-(1,3-dimethyl-1H-pyrazol-4-yl)ethyl]-8-fluoro-6-hydroxy-1,2,3,4-tetrahydroisoquinolin-7-yl}-1λ⁶,2,5-thiadiazolidine-1,1,3-trione;
5-{2-[2-(1,5-dimethyl-1H-pyrazol-4-yl)ethyl]-8-fluoro-6-hydroxy-1,2,3,4-tetrahydroisoquinolin-7-yl}-1λ⁶,2,5-thiadiazolidine-1,1,3-trione;
N-(cyclopropylmethyl)-8-fluoro-6-hydroxy-7-(1,1,4-trioxo-1λ⁶,2,5-thiadiazolidin-2-yl)-3,4-dihydroisoquinoline-2(1H)-carboxamide;
5-{(7S)-7-[(3,3-difluoropropyl)amino]-1-fluoro-3-hydroxy-5,6,7,8-tetrahydronaphthalen-2-yl}-1λ⁶,2,5-thiadiazolidine-1,1,3-trione;
5-{(7R)-7-[(3,3-difluoropropyl)amino]-1-fluoro-3-hydroxy-5,6,7,8-tetrahydronaphthalen-2-yl}-1λ⁶,2,5-thiadiazolidine-1,1,3-trione;
8-fluoro-6-hydroxy-N-[(oxan-4-yl)methyl]-7-(1,1,4-trioxo-1λ⁶,2,5-thiadiazolidin-2-yl)-3,4-dihydroisoquinoline-2(1H)-carboxamide;
N-[(3,3-difluorocyclobutyl)methyl]-8-fluoro-6-hydroxy-7-(1,1,4-trioxo-1λ⁶,2,5-thiadiazolidin-2-yl)-3,4-dihydroisoquinoline-2(1H)-carboxamide;
8-fluoro-6-hydroxy-N-[(oxolan-2-yl)methyl]-7-(1,1,4-trioxo-1λ⁶,2,5-thiadiazolidin-2-yl)-3,4-dihydroisoquinoline-2(1H)-carboxamide;
N-(2-cyclopropylethyl)-8-fluoro-6-hydroxy-7-(1,1,4-trioxo-1λ⁶,2,5-thiadiazolidin-2-yl)-3,4-dihydroisoquinoline-2(1H)-carboxamide;
N-[(1,3-dimethyl-1H-pyrazol-5-yl)methyl]-8-fluoro-6-hydroxy-7-(1,1,4-trioxo-1λ⁶,2,5-thiadiazolidin-2-yl)-3,4-dihydroisoquinoline-2(1H)-carboxamide;
5-{2-[2-(1-tert-butyl-3,5-dimethyl-1H-pyrazol-4-yl)ethyl]-8-fluoro-6-hydroxy-1,2,3,4-tetrahydroisoquinolin-7-yl}-1λ⁶,2,5-thiadiazolidine-1,1,3-trione;
5-[2-(aminomethyl)-4-fluoro-6-hydroxy-2,3-dihydro-1H-inden-5-yl]-1λ⁶,2,5-thiadiazolidine-1,1,3-trione;
5-(4-fluoro-6-hydroxy-2-{[(3-methylbutyl)amino]methyl}-2,3-dihydro-1H-inden-5-yl)-1λ⁶,2,5-thiadiazolidine-1,1,3-trione;
5-(2-{[bis(3-methylbutyl)amino]methyl}-4-fluoro-6-hydroxy-2,3-dihydro-1H-inden-5-yl)-1λ⁶,2,5-thiadiazolidine-1,1,3-trione
8-fluoro-6-hydroxy-N-[(oxolan-3-yl)methyl]-7-(1,1,4-trioxo-1λ⁶,2,5-thiadiazolidin-2-yl)-3,4-dihydroisoquinoline-2(1H)-carboxamide;
N-[(1,5-dimethyl-1H-pyrazol-4-yl)methyl]-8-fluoro-6-hydroxy-7-(1,1,4-trioxo-1λ⁶,2,5-thiadiazolidin-2-yl)-3,4-dihydroisoquinoline-2(1H)-carboxamide;
8-fluoro-6-hydroxy-N-[(1-methyl-1H-pyrazol-5-yl)methyl]-7-(1,1,4-trioxo-1λ⁶,2,5-thiadiazolidin-2-yl)-3,4-dihydroisoquinoline-2(1H)-carboxamide;
5-(2-{2-[3,5-dimethyl-1-(propan-2-yl)-1H-pyrazol-4-yl]ethyl}-8-fluoro-6-hydroxy-1,2,3,4-tetrahydroisoquinolin-7-yl)-1λ⁶,2,5-thiadiazolidine-1,1,3-trione;
5-(2-{[(3-cyclopropylpropyl)amino]methyl}-4-fluoro-6-hydroxy-2,3-dihydro-1H-inden-5-yl)-1λ⁶,2,5-thiadiazolidine-1,1,3-trione;
5-(4-fluoro-6-hydroxy-2-{[(2-methylpropyl)amino]methyl}-2,3-dihydro-1H-inden-5-yl)-1λ⁶,2,5-thiadiazolidine-1,1,3-trione;
5-{2-[2-(1-ethyl-3,5-dimethyl-1H-pyrazol-4-yl)ethyl]-8-fluoro-6-hydroxy-1,2,3,4-tetrahydroisoquinolin-7-yl}-1λ⁶,2,5-thiadiazolidine-1,1,3-trione;
N-(2,2-dimethylpropyl)-8-fluoro-6-hydroxy-7-(1,1,4-trioxo-1λ⁶,2,5-thiadiazolidin-2-yl)-3,4-dihydroisoquinoline-2(1H)-carboxamide;
8-fluoro-6-hydroxy-N-(3-methoxypropyl)-7-(1,1,4-trioxo-1λ⁶,2,5-thiadiazolidin-2-yl)-3,4-dihydroisoquinoline-2(1H)-carboxamide;
8-fluoro-6-hydroxy-N-(3-methoxy-2,2-dimethylpropyl)-7-(1,1,4-trioxo-1λ⁶,2,5-thiadiazolidin-2-yl)-3,4-dihydroisoquinoline-2(1H)-carboxamide;
N-[2-(dimethylamino)ethyl]-8-fluoro-6-hydroxy-7-(1,1,4-trioxo-1λ⁶,2,5-thiadiazolidin-2-yl)-3,4-dihydroisoquinoline-2(1H)-carboxamide;
8-fluoro-6-hydroxy-N-[2-(1-methylcyclopropyl)ethyl]-7-(1,1,4-trioxo-1λ⁶,2,5-thiadiazolidin-2-yl)-3,4-dihydroisoquinoline-2(1H)-carboxamide;
8-fluoro-6-hydroxy-N-(2-methoxyethyl)-7-(1,1,4-trioxo-1λ⁶,2,5-thiadiazolidin-2-yl)-3,4-dihydroisoquinoline-2(1H)-carboxamide;
8-fluoro-6-hydroxy-N-[(oxetan-3-yl)methyl]-7-(1,1,4-trioxo-1λ⁶,2,5-thiadiazolidin-2-yl)-3,4-dihydroisoquinoline-2(1H)-carboxamide;
8-fluoro-6-hydroxy-N-(2-phenylethyl)-7-(1,1,4-trioxo-1λ⁶,2,5-thiadiazolidin-2-yl)-3,4-dihydroisoquinoline-2(1H)-carboxamide;
N-[3-(dimethylamino) propyl]-8-fluoro-6-hydroxy-7-(1,1,4-trioxo-1λ⁶,2,5-thiadiazolidin-2-yl)-3,4-dihydroisoquinoline-2(1H)-carboxamide;
5-[2-(3-cyclohexylpropyl)-8-fluoro-6-hydroxy-1,2,3,4-tetrahydroisoquinolin-7-yl]-1λ⁶,2,5-thiadiazolidine-1,1,3-trione;
5-(7-{[(3,5-dimethyl-1,2-oxazol-4-yl)methyl]amino}-1-fluoro-3-hydroxy-5,6,7,8-tetrahydronaphthalen-2-yl)-1λ⁶,2,5-thiadiazolidine-1,1,3-trione;
5-{2-[3-(2,2-dimethylcyclopropyl)propyl]-8-fluoro-6-hydroxy-1,2,3,4-tetrahydroisoquinolin-7-yl}-1λ⁶,2,5-thiadiazolidine-1,1,3-trione;
5-[(3S)-5-fluoro-7-hydroxy-3-methyl-1,2,3,4-tetrahydroisoquinolin-6-yl]-1λ⁶,2,5-thiadiazolidine-1,1,3-trione;
5-(7-{[2-(3,5-dimethyl-1,2-oxazol-4-yl)ethyl]amino}-1-fluoro-3-hydroxy-5,6,7,8-tetrahydronaphthalen-2-yl)-1λ⁶,2,5-thiadiazolidine-1,1,3-trione;
5-[(2R)-4-fluoro-6-hydroxy-2-{[(3-methylbutyl)amino]methyl}-2,3-dihydro-1H-inden-5-yl]-1λ⁶,2,5-thiadiazolidine-1,1,3-trione;
5-[(2S)-4-fluoro-6-hydroxy-2-{[(3-methylbutyl)amino]methyl}-2,3-dihydro-1H-inden-5-yl]-1λ⁶,2,5-thiadiazolidine-1,1,3-trione;
5-[8-fluoro-6-hydroxy-2-(4-methoxybutyl)-1,2,3,4-tetrahydroisoquinolin-7-yl]-1λ⁶,2,5-thiadiazolidine-1,1,3-trione;
5-(8-fluoro-6-hydroxy-2-{3-[3-(trifluoromethyl)phenyl]propyl}-1,2,3,4-tetrahydroisoquinolin-7-yl)-1λ⁶,2,5-thiadiazolidine-1,1,3-trione;
5-(8-fluoro-6-hydroxy-2-{2-methyl-3-[4-(propan-2-yl)phenyl]propyl}-1,2,3,4-tetrahydroisoquinolin-7-yl)-1λ⁶,2,5-thiadiazolidine-1,1,3-trione;
5-{2-[4-(5,5-dimethyl-1,3-dioxan-2-yl)butyl]-8-fluoro-6-hydroxy-1,2,3,4-tetrahydroisoquinolin-7-yl}-1λ⁶,2,5-thiadiazolidine-1,1,3-trione;
5-{8-fluoro-6-hydroxy-2-[2-(2,6,6-trimethylcyclohex-1-en-1-yl)ethyl]-1,2,3,4-tetrahydroisoquinolin-7-yl}-1λ⁶,2,5-thiadiazolidine-1,1,3-trione;
5-(8-fluoro-6-hydroxy-2-pentyl-1,2,3,4-tetrahydroisoquinolin-7-yl)-1λ⁶,2,5-thiadiazolidine-1,1,3-trione;
5-{8-fluoro-2-[3-(4-fluorophenyl)propyl]-6-hydroxy-1,2,3,4-tetrahydroisoquinolin-7-yl}-1λ⁶,2,5-thiadiazolidine-1,1,3-trione;
tert-butyl [(1r,4r)-4-{2-[8-fluoro-6-hydroxy-7-(1,1,4-trioxo-1λ⁶,2,5-thiadiazolidin-2-yl)-3,4-dihydroisoquinolin-2(1H)-yl]ethyl}cyclohexyl]carbamate;
5-{2-[3-(4-tert-butylphenyl)propyl]-8-fluoro-6-hydroxy-1,2,3,4-tetrahydroisoquinolin-7-yl}-1λ⁶,2,5-thiadiazolidine-1,1,3-trione;
5-[8-fluoro-6-hydroxy-2-(3,5,5-trimethylhexyl)-1,2,3,4-tetrahydroisoquinolin-7-yl]-1λ⁶,2,5-thiadiazolidine-1,1,3-trione;
5-{8-fluoro-2-[3-(2-fluorophenyl)propyl]-6-hydroxy-1,2,3,4-tetrahydroisoquinolin-7-yl}-1λ⁶,2,5-thiadiazolidine-1,1,3-trione;
3-hydroxybutyl 4,4,8-trifluoro-6-hydroxy-7-(1,1,4-trioxo-1λ⁶,2,5-thiadiazolidin-2-yl)-3,4-dihydroisoquinoline-2(1H)-carboxylate;
5-(2-{[(2-cyclobutylethyl)amino]methyl}-4-fluoro-6-hydroxy-2,3-dihydro-1H-inden-5-yl)-1λ⁶,2,5-thiadiazolidine-1,1,3-trione;
5-{2-[2-(3,5-dimethyl-1,2-oxazol-4-yl)ethyl]-4,4,8-trifluoro-6-hydroxy-1,2,3,4-tetrahydroisoquinolin-7-yl}-1λ⁶,2,5-thiadiazolidine-1,1,3-trione;
5-[(7R)-1-fluoro-3-hydroxy-7-{[(3R)-3-hydroxybutyl]amino}-5,6,7,8-tetrahydronaphthalen-2-yl]-1λ⁶,2,5-thiadiazolidine-1,1,3-trione;
5-{(7R)-1-fluoro-3-hydroxy-7-[(4-hydroxy-3,3-dimethylbutyl)amino]-5,6,7,8-tetrahydronaphthalen-2-yl}-1λ⁶,2,5-thiadiazolidine-1,1,3-trione;
5-[1-fluoro-3-hydroxy-6-(3-hydroxy-3-methylbutoxy)-5,6,7,8-tetrahydronaphthalen-2-yl]-1λ⁶,2,5-thiadiazolidine-1,1,3-trione;
5-[6-(cyclopropylmethoxy)-1-fluoro-3-hydroxy-5,6,7,8-tetrahydronaphthalen-2-yl]-1λ⁶,2,5-thiadiazolidine-1,1,3-trione;
5-{6-[(4,4-difluorobutyl)amino]-1-fluoro-3-hydroxy-5,6,7,8-tetrahydronaphthalen-2-yl}-1λ⁶,2,5-thiadiazolidine-1,1,3-trione;
5-[6-(4,4-difluorobutoxy)-1-fluoro-3-hydroxy-5,6,7,8-tetrahydronaphthalen-2-yl]-1λ⁶,2,5-thiadiazolidine-1,1,3-trione;
5-{1-fluoro-3-hydroxy-6-[(3-methylbutyl)amino]-5,6,7,8-tetrahydronaphthalen-2-yl}-1λ⁶,2,5-thiadiazolidine-1,1,3-trione;
5-[1-fluoro-3-hydroxy-6-(3-methylbutoxy)-5,6,7,8-tetrahydronaphthalen-2-yl]-1λ⁶,2,5-thiadiazolidine-1,1,3-trione;
5-{1-fluoro-3-hydroxy-6-[(3-hydroxy-3-methylbutyl)amino]-5,6,7,8-tetrahydronaphthalen-2-yl}-1λ⁶,2,5-thiadiazolidine-1,1,3-trione;
tert-butyl (2-{[5-fluoro-7-hydroxy-6-(1,1,4-trioxo-1λ⁶,2,5-thiadiazolidin-2-yl)-1,2,3,4-tetrahydronaphthalen-2-yl]oxy}ethyl)carbamate;
5-(1-fluoro-3-hydroxy-6-methoxy-5,6,7,8-tetrahydronaphthalen-2-yl)-1λ⁶,2,5-thiadiazolidine-1,1,3-trione;
5-{6-[(cyclopropylmethyl)amino]-1-fluoro-3-hydroxy-5,6,7,8-tetrahydronaphthalen-2-yl}-1λ⁶,2,5-thiadiazolidine-1,1,3-trione;
5-[6-(2-aminoethoxy)-1-fluoro-3-hydroxy-5,6,7,8-tetrahydronaphthalen-2-yl]-1λ⁶,2,5-thiadiazolidine-1,1,3-trione;
5-{2-[2-(3,5-dimethyl-1H-pyrazol-4-yl)ethyl]-4,4,8-trifluoro-6-hydroxy-1,2,3,4-tetrahydroisoquinolin-7-yl}-1λ⁶,2,5-thiadiazolidine-1,1,3-trione;
N-(cyclohexylmethyl)-8-fluoro-6-hydroxy-7-(1,1,4-trioxo-1λ⁶,2,5-thiadiazolidin-2-yl)-3,4-dihydroisoquinoline-2(1H)-carboxamide;
N-[8-fluoro-6-hydroxy-7-(1,1,4-trioxo-1λ⁶,2,5-thiadiazolidin-2-yl)-1,2,3,4-tetrahydronaphthalen-2-yl]acetamide;
5-[1-fluoro-3-hydroxy-7-(4-methylpentyl)-5,6,7,8-tetrahydronaphthalen-2-yl]-1λ⁶,2,5-thiadiazolidine-1,1,3-trione;
5-(8-fluoro-6-hydroxy-2-{[(2S)-5-oxopyrrolidin-2-yl]methyl}-1,2,3,4-tetrahydroisoquinolin-7-yl)-1λ⁶,2,5-thiadiazolidine-1,1,3-trione;
5-[(3S)-5-fluoro-7-hydroxy-3-(4-methylpentyl)-1,2,3,4-tetrahydroisoquinolin-6-yl]-1λ⁶,2,5-thiadiazolidine-1,1,3-trione;
5-[(3S)-3-amino-5-fluoro-7-hydroxy-3,4-dihydro-2H-1-benzopyran-6-yl]-1λ⁶,2,5-thiadiazolidine-1,1,3-trione;
5-{(7R)-1-fluoro-3-hydroxy-7-[(3-methylbutyl)amino]-5,6,7,8-tetrahydronaphthalen-2-yl}(4,4-²H₂)-1λ⁶,2,5-thiadiazolidine-1,1,3-trione;
8-fluoro-6-hydroxy-N-(2-methylpropyl)-7-(1,1,4-trioxo-1λ⁶,2,5-thiadiazolidin-2-yl)-3,4-dihydroisoquinoline-2(1H)-sulfonamide;
8-fluoro-6-hydroxy-N-(2-methylpropyl)-7-(1,1,4-trioxo-1λ⁶,2,5-thiadiazolidin-2-yl)-3,4-dihydroisoquinoline-2(1H)-carboximidamide;
5-(1-fluoro-3-hydroxy-7-{[2-(oxetan-3-yl)ethyl]amino}-5,6,7,8-tetrahydronaphthalen-2-yl)-1λ⁶,2,5-thiadiazolidine-1,1,3-trione;
5-{(7R)-1,4-difluoro-3-hydroxy-7-[(3-methylbutyl)amino]-5,6,7,8-tetrahydronaphthalen-2-yl}-1λ⁶,2,5-thiadiazolidine-1,1,3-trione;
N-[8-fluoro-6-hydroxy-7-(1,1,4-trioxo-1λ⁶,2,5-thiadiazolidin-2-yl)-1,2,3,4-tetrahydronaphthalen-2-yl]-3-methylbutane-1-sulfonamide;
5-(1-fluoro-3-hydroxy-7-{[(2-methylpropyl)amino]methyl}-5,6,7,8-tetrahydronaphthalen-2-yl)-1λ⁶,2,5-thiadiazolidine-1,1,3-trione;
5-{1-fluoro-7-[(2-fluoro-3-methylbutyl)amino]-3-hydroxy-5,6,7,8-tetrahydronaphthalen-2-yl}-1λ⁶,2,5-thiadiazolidine-1,1,3-trione;
5-(1-fluoro-3,7-dihydroxy-5,6,7,8-tetrahydronaphthalen-2-yl)-1λ⁶,2,5-thiadiazolidine-1,1,3-trione;
5-{7-[(²H₉)butylamino]-1-fluoro-3-hydroxy-5,6,7,8-tetrahydronaphthalen-2-yl}-1λ⁶,2,5-thiadiazolidine-1,1,3-trione;
5-[7-(aminomethyl)-1-fluoro-3,7-dihydroxy-5,6,7,8-tetrahydronaphthalen-2-yl]-1λ⁶,2,5-thiadiazolidine-1,1,3-trione;
5-[(7R)-1-fluoro-3-hydroxy-7-({2-[1-(hydroxymethyl)cyclobutyl]ethyl}amino)-5,6,7,8-tetrahydronaphthalen-2-yl]-1λ⁶,2,5-thiadiazolidine-1,1,3-trione;
5-(1-fluoro-3,7-dihydroxy-7-{[(2-methylpropyl)amino]methyl}-5,6,7,8-tetrahydronaphthalen-2-yl)-1λ⁶,2,5-thiadiazolidine-1,1,3-trione;
5-{1-fluoro-3-hydroxy-7-[(3-methylbutyl)amino](6,6,7,8,8-²H₅)-5,6,7,8-tetrahydronaphthalen-2-yl}-1λ⁶,2,5-thiadiazolidine-1,1,3-trione;
tert-butyl [(2R)-8-fluoro-6-hydroxy-7-(1,1,4-trioxo-1λ⁶,2,5-thiadiazolidin-2-yl)-1,2,3,4-tetrahydronaphthalen-2-yl]carbamate;
5-[(7R)-1-fluoro-3-hydroxy-7-{[(thiophen-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(thiophen-2-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(3-methyloxetan-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(1-methyl-1H-pyrrol-2-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(1-methyl-1H-pyrrol-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(pyridin-3-yl)methyl]amino}-5,6,7,8-tetrahydronaphthalen-2-yl]-1λ⁶,2,5-thiadiazolidine-1,1,3-trione;
5-{(7R)-1-fluoro-3-hydroxy-7-[(3,3,3-trifluoro-2-methylpropyl)amino]-5,6,7,8-tetrahydronaphthalen-2-yl}-1λ⁶,2,5-thiadiazolidine-1,1,3-trione;
5-[(7R)-1-fluoro-3-hydroxy-7-{[(pyridazin-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(oxan-2-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(5-methyl-1,2-oxazol-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(oxan-3-yl)methyl]amino}-5,6,7,8-tetrahydronaphthalen-2-yl]-1λ⁶,2,5-thiadiazolidine-1,1,3-trione;
2-({[(2R)-8-fluoro-6-hydroxy-7-(1,1,4-trioxo-1λ⁶,2,5-thiadiazolidin-2-yl)-1,2,3,4-tetrahydronaphthalen-2-yl]amino}methyl)cyclopropane-1-carbonitrile;
5-{(7R)-7-[(3-ethoxypropyl)amino]-1-fluoro-3-hydroxy-5,6,7,8-tetrahydronaphthalen-2-yl}-1λ⁶,2,5-thiadiazolidine-1,1,3-trione;
5-[(7R)-7-({[1-(difluoromethyl)cyclopropyl]methyl}amino)-1-fluoro-3-hydroxy-5,6,7,8-tetrahydronaphthalen-2-yl]-1λ⁶,2,5-thiadiazolidine-1,1,3-trione;
5-[(7R)-1-fluoro-3-hydroxy-7-{[2-(oxolan-3-yl)ethyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(1-methyl-1H-imidazol-5-yl)methyl]amino}-5,6,7,8-tetrahydronaphthalen-2-yl]-1λ⁶,2,5-thiadiazolidine-1,1,3-trione;
5-[(7R)-7-{[2,2-dimethyl-3-(pyrrolidin-1-yl)propyl]amino}-1-fluoro-3-hydroxy-5,6,7,8-tetrahydronaphthalen-2-yl]-1λ⁶,2,5-thiadiazolidine-1,1,3-trione;
5-[(7R)-1-fluoro-3-hydroxy-7-({[5-(hydroxymethyl)furan-2-yl]methyl}amino)-5,6,7,8-tetrahydronaphthalen-2-yl]-1λ⁶,2,5-thiadiazolidine-1,1,3-trione;
5-{(7R)-1-fluoro-3-hydroxy-7-[(4-methoxy butyl)amino]-5,6,7,8-tetrahydronaphthalen-2-yl}-1λ⁶,2,5-thiadiazolidine-1,1,3-trione;
5-[(7R)-1-fluoro-3-hydroxy-7-{[(oxolan-3-yl)methyl]amino}-5,6,7,8-tetrahydronaphthalen-2-yl]-1λ⁶,2,5-thiadiazolidine-1,1,3-trione;
5-[(7R)-7-{[(2,2-difluorocyclopropyl)methyl]amino}-1-fluoro-3-hydroxy-5,6,7,8-tetrahydronaphthalen-2-yl]-1λ⁶,2,5-thiadiazolidine-1,1,3-trione;
5-{(7R)-1-fluoro-3-hydroxy-7-[(3-methoxypropyl)amino]-5,6,7,8-tetrahydronaphthalen-2-yl}-1λ⁶,2,5-thiadiazolidine-1,1,3-trione;
5-[(7R)-1-fluoro-3-hydroxy-7-{[(1,3-oxazol-5-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[2-(oxan-4-yl)ethyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(oxetan-3-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(1,3-thiazol-2-yl)methyl]amino}-5,6,7,8-tetrahydronaphthalen-2-yl]-1λ⁶,2,5-thiadiazolidine-1,1,3-trione;
5-[(7R)-1-fluoro-3-hydroxy-7-{[(pyridazin-4-yl)methyl]amino}-5,6,7,8-tetrahydronaphthalen-2-yl]-1λ⁶,2,5-thiadiazolidine-1,1,3-trione;
5-{(7R)-1-fluoro-3-hydroxy-7-[(3-hydroxybutyl)amino]-5,6,7,8-tetrahydronaphthalen-2-yl}-1λ⁶,2,5-thiadiazolidine-1,1,3-trione;
5-[(7S)-1-fluoro-3-hydroxy-7-[(3-methylbutyl)amino](6,6,7,8,8-²H₅)-5,6,7,8-tetrahydronaphthalen-2-yl]-1λ⁶,2,5-thiadiazolidine-1,1,3-trione;
5-[(7R)-1-fluoro-3-hydroxy-7-[(3-methylbutyl)amino](6,6,7,8,8-²H₅)-5,6,7,8-tetrahydronaphthalen-2-yl]-1λ⁶,2,5-thiadiazolidine-1,1,3-trione;
5-{(3S)-5-fluoro-7-hydroxy-3-[(3-methylbutyl)amino]-3,4-dihydro-2H-1-benzopyran-6-yl}-1λ⁶,2,5-thiadiazolidine-1,1,3-trione;
5-{(3S)-3-[(4,4-difluorobutyl)amino]-5-fluoro-7-hydroxy-3,4-dihydro-2H-1-benzopyran-6-yl}-1λ⁶,2,5-thiadiazolidine-1,1,3-trione;
5-{(7R)-7-[(5-amino-3,3-dimethylpentyl)amino]-1-fluoro-3-hydroxy-5,6,7,8-tetrahydronaphthalen-2-yl}-1λ⁶,2,5-thiadiazolidine-1,1,3-trione;
5-[(3S)-5-fluoro-7-hydroxy-3-{[2-(oxan-4-yl)ethyl]amino}-3,4-dihydro-2H-1-benzopyran-6-yl]-1λ⁶,2,5-thiadiazolidine-1,1,3-trione;
5-[(7R)-7-({2-[1-(aminomethyl)cyclobutyl]ethyl}amino)-1-fluoro-3-hydroxy-5,6,7,8-tetrahydronaphthalen-2-yl]-1λ⁶,2,5-thiadiazolidine-1,1,3-trione;
5-[(7R)-7-({2-[1-(2-aminoethyl)cyclobutyl]ethyl}amino)-1-fluoro-3-hydroxy-5,6,7,8-tetrahydronaphthalen-2-yl]-1λ⁶,2,5-thiadiazolidine-1,1,3-trione;
5-[(3S)-5-fluoro-7-hydroxy-3-{[2-(2,6,6-trimethylcyclohex-1-en-1-yl)ethyl]amino}-3,4-dihydro-2H-1-benzopyran-6-yl]-1λ⁶,2,5-thiadiazolidine-1,1,3-trione;
5-[(3S)-3-{[3-(2,2-difluoroethoxy)propyl]amino}-5-fluoro-7-hydroxy-3,4-dihydro-2H-1-benzopyran-6-yl]-1λ⁶,2,5-thiadiazolidine-1,1,3-trione;
5-[(3S)-5-fluoro-7-hydroxy-3-({[4-(trifluoromethyl)cyclohexyl]methyl}amino)-3,4-dihydro-2H-1-benzopyran-6-yl]-1λ⁶,2,5-thiadiazolidine-1,1,3-trione;
5-[(3S)-5-fluoro-3-({[1-(fluoromethyl)cyclopropyl]methyl}amino)-7-hydroxy-3,4-dihydro-2H-1-benzopyran-6-yl]-1λ⁶,2,5-thiadiazolidine-1,1,3-trione;
5-[(3S)-5-fluoro-7-hydroxy-3-{[2-(oxolan-3-yl)ethyl]amino}-3,4-dihydro-2H-1-benzopyran-6-yl]-1λ⁶,2,5-thiadiazolidine-1,1,3-trione;
5-[(3S)-3-({[(1RS,5SR)-bicyclo[3.1.0]hexan-6-yl]methyl}amino)-5-fluoro-7-hydroxy-3,4-dihydro-2H-1-benzopyran-6-yl]-1λ⁶,2,5-thiadiazolidine-1,1,3-trione;
tert-butyl 4-({[(3S)-5-fluoro-7-hydroxy-6-(1,1,4-trioxo-1λ⁶,2,5-thiadiazolidin-2-yl)-3,4-dihydro-2H-1-benzopyran-3-yl]amino}methyl)piperidine-1-carboxylate;
5-[(3S)-5-fluoro-7-hydroxy-3-{[(3-phenylcyclobutyl)methyl]amino}-3,4-dihydro-2H-1-benzopyran-6-yl]-1λ⁶,2,5-thiadiazolidine-1,1,3-trione;
5-{(3S)-5-fluoro-7-hydroxy-3-[(3-phenylpropyl)amino]-3,4-dihydro-2H-1-benzopyran-6-yl}-1λ⁶,2,5-thiadiazolidine-1,1,3-trione;
5-[8-fluoro-6-hydroxy-2-(4-methylpentyl)-1-oxo-1,2,3,4-tetrahydroisoquinolin-7-yl]-1λ⁶,2,5-thiadiazolidine-1,1,3-trione;
5-(8-fluoro-6-hydroxy-1-oxo-1,2,3,4-tetrahydroisoquinolin-7-yl)-1λ⁶,2,5-thiadiazolidine-1,1,3-trione;
5-[7-(aminomethyl)-1-fluoro-3-hydroxy-5,6,7,8-tetrahydronaphthalen-2-yl]-1λ⁶,2,5-thiadiazolidine-1,1,3-trione;
5-(1-fluoro-3-hydroxy-7-{[(3-methylbutyl)amino]methyl}-5,6,7,8-tetrahydronaphthalen-2-yl)-1λ⁶,2,5-thiadiazolidine-1,1,3-trione;
tert-butyl {[8-fluoro-6-hydroxy-7-(1,1,4-trioxo-1λ⁶,2,5-thiadiazolidin-2-yl)-1,2,3,4-tetrahydronaphthalen-2-yl]methyl}carbamate;
tert-butyl [(2R)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate;
5-{(6R,7S)-1-fluoro-3,6-dihydroxy-7-[(3-methylbutyl)amino]-5,6,7,8-tetrahydronaphthalen-2-yl}-1λ⁶,2,5-thiadiazolidine-1,1,3-trione;
5-(7-{[(3-cyclopropylpropyl)amino]methyl}-1-fluoro-3-hydroxy-5,6,7,8-tetrahydronaphthalen-2-yl)-1λ⁶,2,5-thiadiazolidine-1,1,3-trione;
tert-butyl [(2R,4R)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate:
5-{7-[(butylamino)methyl]-1-fluoro-3-hydroxy-5,6,7,8-tetrahydronaphthalen-2-yl}-1λ⁶,2,5-thiadiazolidine-1,1,3-trione;
5-[(5R,7R)-7-amino-1-fluoro-3-hydroxy-5-methyl-5,6,7,8-tetrahydronaphthalen-2-yl]-1λ⁶,2,5-thiadiazolidine-1,1,3-trione;
5-[(5S,7R)-7-amino-1-fluoro-3-hydroxy-5-methyl-5,6,7,8-tetrahydronaphthalen-2-yl]-1λ⁶,2,5-thiadiazolidine-1,1,3-trione;
5-(7-{[(cyclopropylmethyl)amino]methyl}-1-fluoro-3-hydroxy-5,6,7,8-tetrahydronaphthalen-2-yl)-1λ⁶,2,5-thiadiazolidine-1,1,3-trione;
5-(7-{[(cyclobutylmethyl)amino]methyl}-1-fluoro-3-hydroxy-5,6,7,8-tetrahydronaphthalen-2-yl)-1λ⁶,2,5-thiadiazolidine-1,1,3-trione;
5-[(7R,8R)-7-amino-1-fluoro-3,8-dihydroxy-5,6,7,8-tetrahydronaphthalen-2-yl]-1λ⁶,2,5-thiadiazolidine-1,1,3-trione;
N-[(2R)-8-fluoro-6-hydroxy-7-(1,1,4-trioxo-1λ⁶,2,5-thiadiazolidin-2-yl)-1,2,3,4-tetrahydronaphthalen-2-yl]acetamide;
5-(1-fluoro-3-hydroxy-7-{[(2-hydroxyethyl)amino]methyl}-5,6,7,8-tetrahydronaphthalen-2-yl)-1λ⁶,2,5-thiadiazolidine-1,1,3-trione;
5-[(7S)-7-(aminomethyl)-1-fluoro-3-hydroxy-5,6,7,8-tetrahydronaphthalen-2-yl]-1λ⁶,2,5-thiadiazolidine-1,1,3-trione;
5-[(7R)-7-(aminomethyl)-1-fluoro-3-hydroxy-5,6,7,8-tetrahydronaphthalen-2-yl]-1λ⁶,2,5-thiadiazolidine-1,1,3-trione;
5-{(7R,8R)-1-fluoro-3,8-dihydroxy-7-[(3-methylbutyl)amino]-5,6,7,8-tetrahydronaphthalen-2-yl}-1λ⁶,2,5-thiadiazolidine-1,1,3-trione;
5-[(2S)-2-(aminomethyl)-4-fluoro-6-hydroxy-2,3-dihydro-1H-inden-5-yl]-1λ⁶,2,5-thiadiazolidine-1,1,3-trione;
5-[(2R)-2-(aminomethyl)-4-fluoro-6-hydroxy-2,3-dihydro-1H-inden-5-yl]-1λ⁶,2,5-thiadiazolidine-1,1,3-trione;
5-{(7R)-7-[(5-amino-4,4-difluoropentyl)amino]-1-fluoro-3-hydroxy-5,6,7,8-tetrahydronaphthalen-2-yl}-1λ⁶,2,5-thiadiazolidine-1,1,3-trione;
5-[(7R)-7-(butylamino)-1-fluoro-3-hydroxy-5,6,7,8-tetrahydronaphthalen-2-yl]-1λ⁶,2,5-thiadiazolidine-1,1,3-trione;
5-{(6S,7S)-1-fluoro-3,6-dihydroxy-7-[(3-methylbutyl)amino]-5,6,7,8-tetrahydronaphthalen-2-yl}-1λ⁶,2,5-thiadiazolidine-1,1,3-trione;
   and a pharmaceutically acceptable salt thereof.

In some embodiments, the compound is 5-{(7R)-1-fluoro-3-hydroxy-7-[(3-methylbutyl)amino]-5,6,7,8-tetrahydronaphthalen-2-yl}-1λ⁶,2,5-thiadiazolidine-1,1,3-trione or a pharmaceutically acceptable salt thereof.

In some embodiments, the compound is 5-{(7R)-1-fluoro-3-hydroxy-7-[(3-methylbutyl)amino]-5,6,7,8-tetrahydronaphthalen-2-yl}-1λ⁶,2,5-thiadiazolidine-1,1,3-trione.

In some embodiments, the compound is a pharmaceutically acceptable salt of 5-{(7R)-1-fluoro-3-hydroxy-7-[(3-methylbutyl)amino]-5,6,7,8-tetrahydronaphthalen-2-yl}-1λ⁶,2,5-thiadiazolidine-1,1,3-trione.

In some embodiments, the compound is

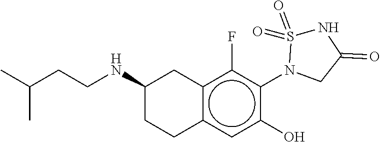

or a pharmaceutically acceptable salt thereof.

In some embodiments, the compound is

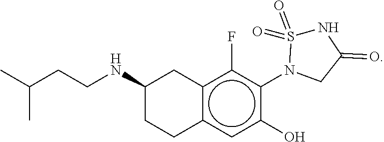

In some embodiments, a compound disclosed herein is formulated as a pharmaceutically acceptable composition comprising a disclosed compound and a pharmaceutically acceptable carrier.

In some embodiments, a compound disclosed herein is formulated for oral administration.

In some embodiments, a compound disclosed herein is selected from a compound set forth in Table 1.

TABLE 1
Exemplary compounds of the disclosure.

| | |
|---|---|
| Com- | |
| pound | |
| Num- | |
| ber | Structure |

| | |
|---|---|
| 100 | 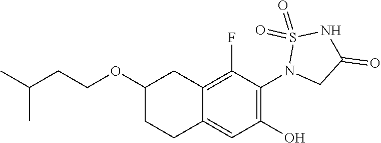 [SMILES: CC(C)CCOC1CCc2cc(O)c(N3CC(=O)NS3(=O)=O)c(F)c2C1] |
| 101 | 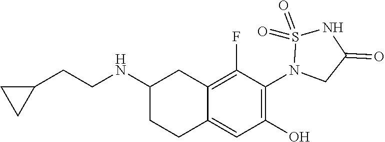 [SMILES: O=C1CN(c2c(O)cc3c(c2F)CC(NCCC2CC2)CC3)S(=O)(=O)N1] |
| 102 | 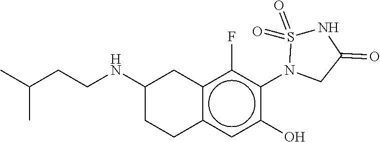 |
| 103 | 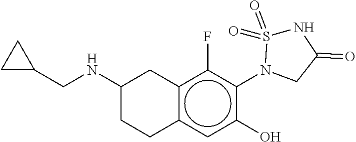 |
| 104 | 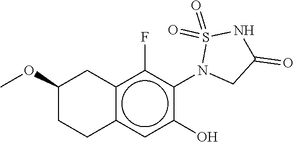 |
| 105 | 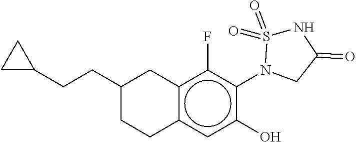 |
| 106 | 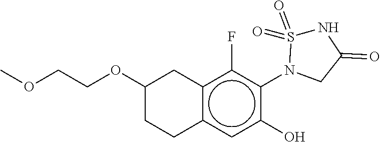 |
| 107 | 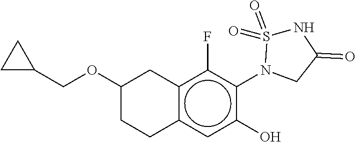 |
| 108 | 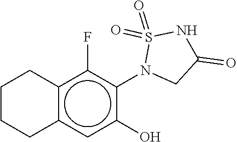 |
| 109 | 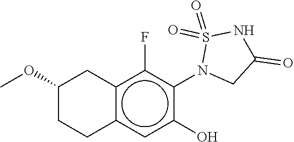 |
| 110 | 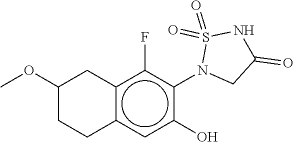 |
| 111 | 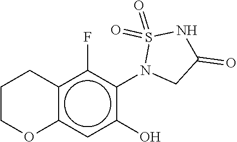 |
| 112 | 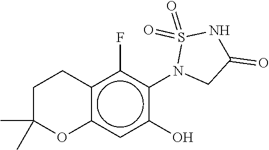 |
| 113 | 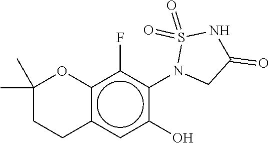 |
| 114 | 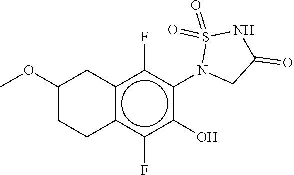 |
| 115 | 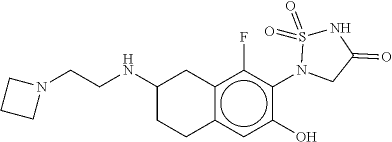 |
| 116 | 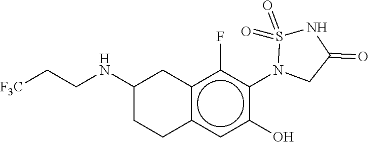 |
| 117 | 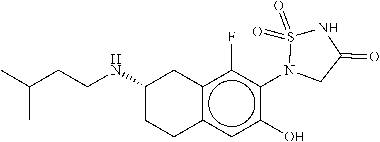 |
| 118 | 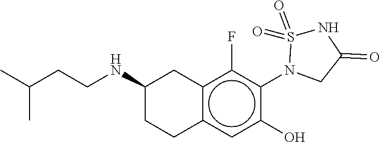 |
| 119 | 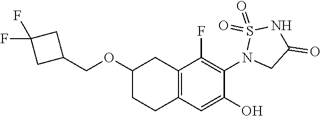 [SMILES: O=C1CN(c2c(O)cc3c(c2F)CC(OCC2CC(F)(F)C2)CC3)S(=O)(=O)N1] |
| 120 | 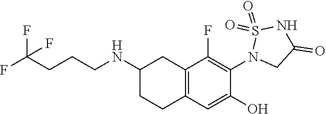 [SMILES: O=C1CN(c2c(O)cc3c(c2F)CC(NCCCC(F)(F)F)CC3)S(=O)(=O)N1] |
| 121 | 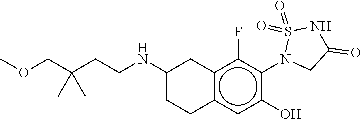 |
| 122 | 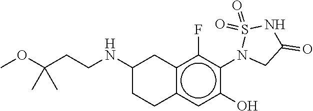 |
| 123 | 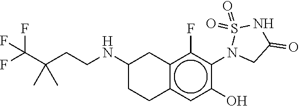 |
| 124 | 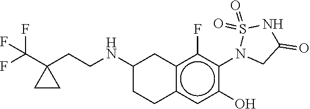 |
| 125 | 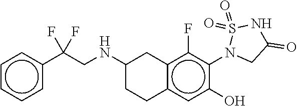 |
| 126 | 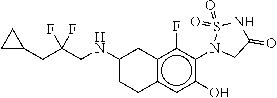 |
| 127 | 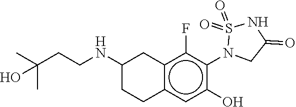 |
| 128 | 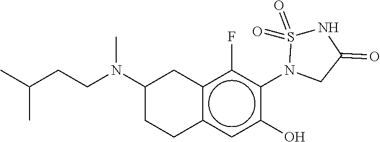 |
| 129 | 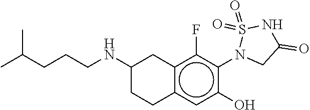 |
| 130 | 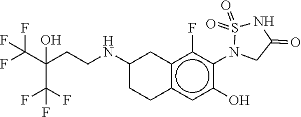 |
| 131 | 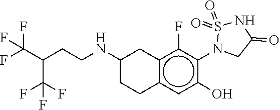 |
| 132 | 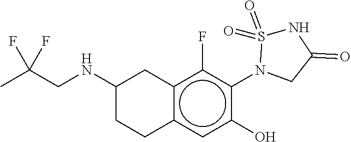 |
| 133 | 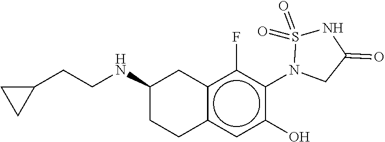 |
| 134 | 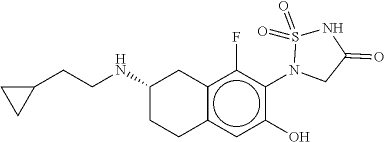 |
| 135 | 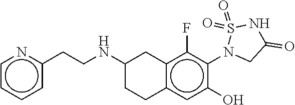 |
| 136 | 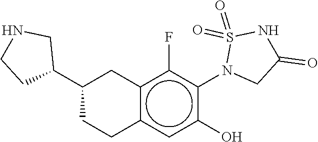 |
| 137 | 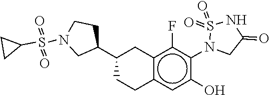 |
| 138 | 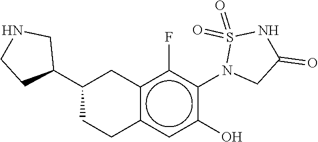 |
| 139 | 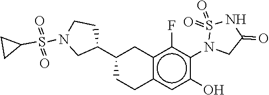 |
| 140 | 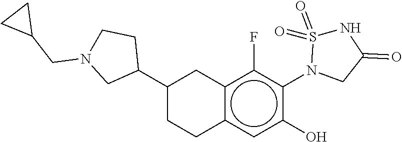 |
| 141 | 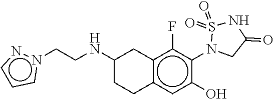 |
| 142 | 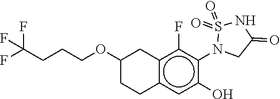 |
| 143 | 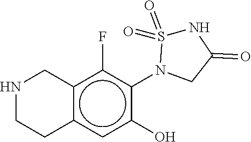 |
| 144 | 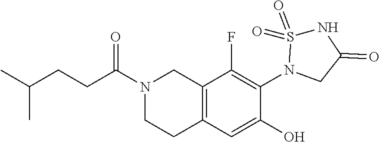 [SMILES: CC(C)CCC(=O)N1CCc2cc(O)c(N3CC(=O)NS3(=O)=O)c(F)c2C1] |
| 145 | 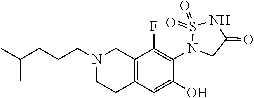 [SMILES: CC(C)CCCN1CCc2cc(O)c(N3CC(=O)NS3(=O)=O)c(F)c2C1] |
| 146 | 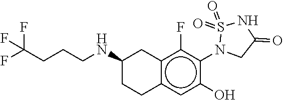 |
| 147 | 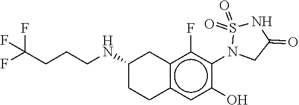 |
| 148 | 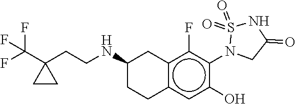 |
| 149 | 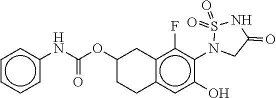 |
| 150 | 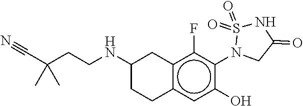 |
| 151 | 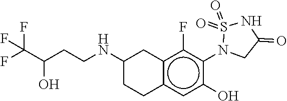 |
| 152 | 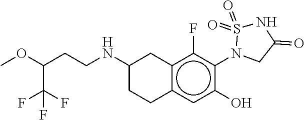 |
| 153 | 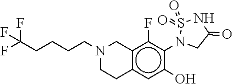 |
| 154 | 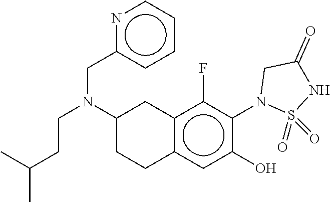 |
| 155 | 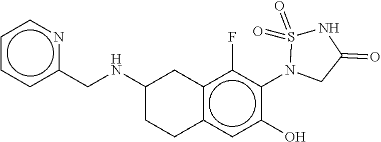 |
| 156 | 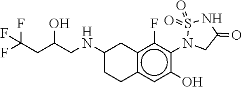 |
| 157 | 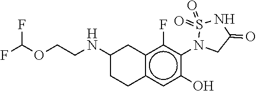 |
| 158 | 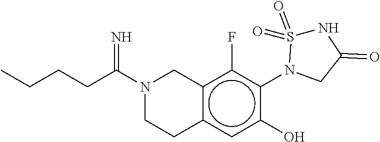 |
| 159 | 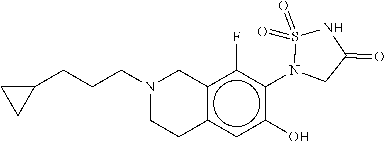 |
| 160 | 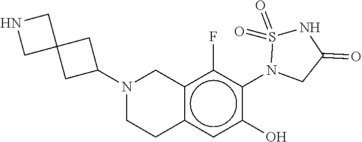 |
| 161 | 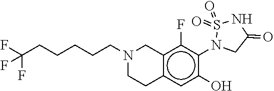 |
| 162 | 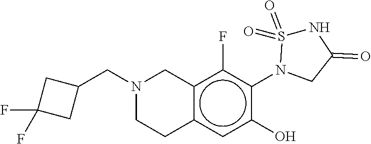 |
| 163 | 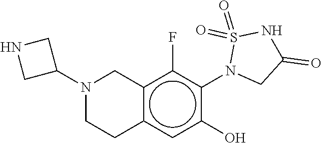 |
| 164 | 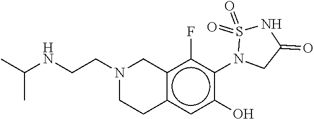 |
| 165 | 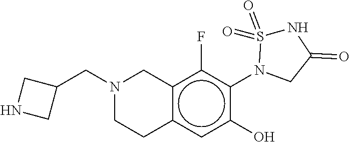 |
| 166 | 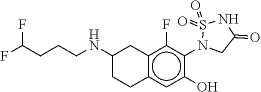 |
| 167 | 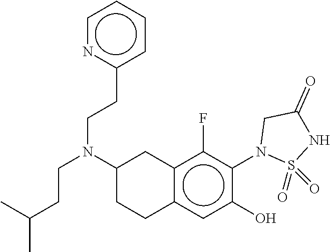 |
| 168 | 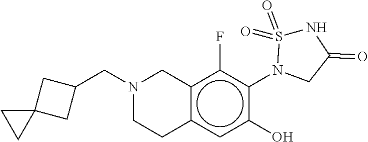 |
| 169 | 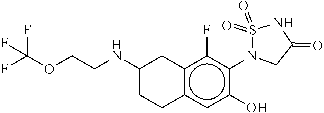 |
| 170 | 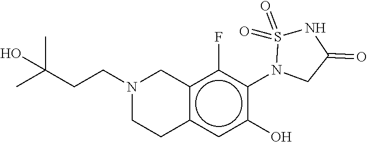 |
| 171 | 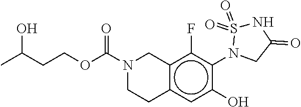 |
| 172 | 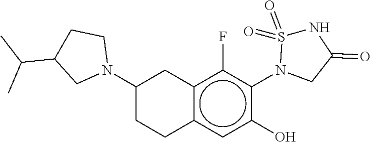 |
| 173 | 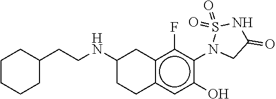 |
| 174 | 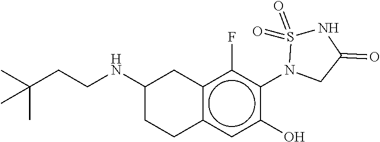 |
| 175 | 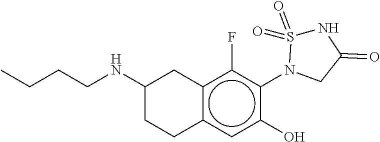 |
| 176 | 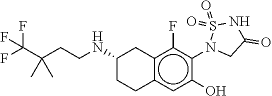 |
| 177 | 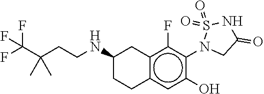 |
| 178 | 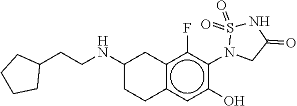 |
| 179 | 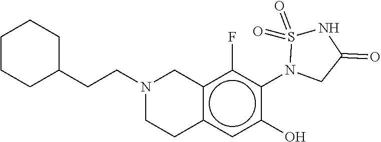 |
| 180 | 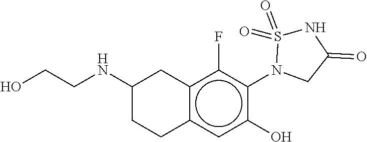 |
| 181 | 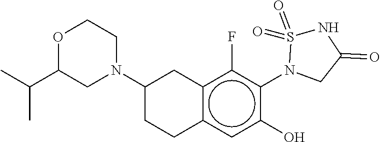 |
| 182 | 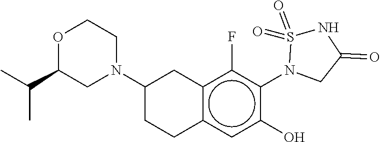 |
| 183 | 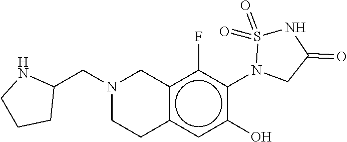 |
| 184 | 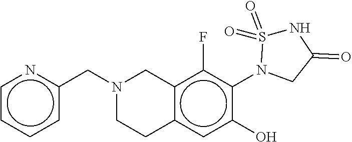 |
| 185 | 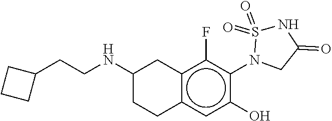 |
| 186 | 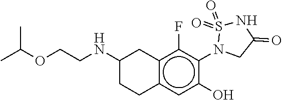 |
| 187 | 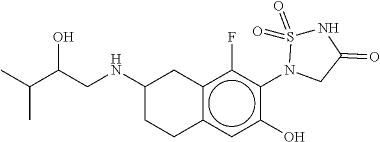 |
| 188 | 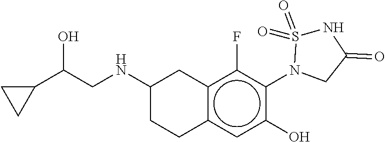 |
| 189 | 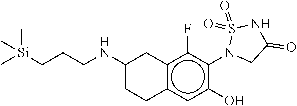 |
| 190 | 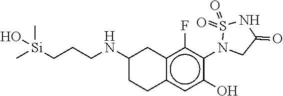 |
| 191 | 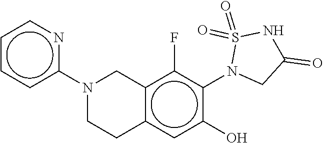 |
| 192 | 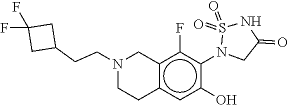 |
| 193 | 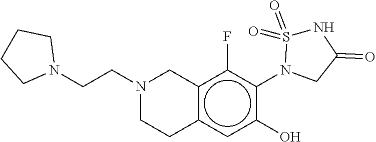 |
| 194 | 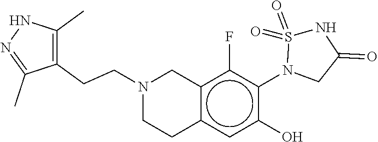 |
| 195 | 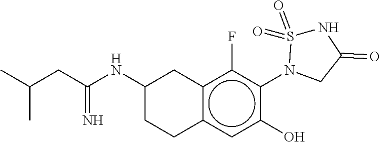 |
| 196 | 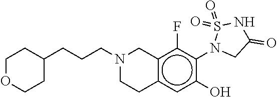 |
| 197 | 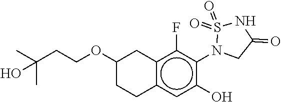 |
| 198 | 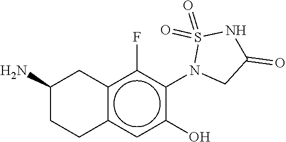 |
| 199 | 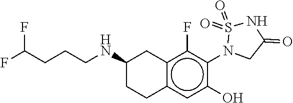 |
| 200 | 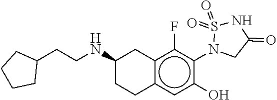 |
| 201 | 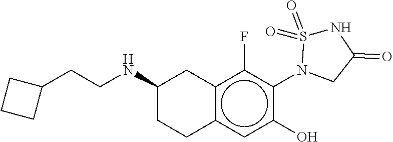 |
| 202 | 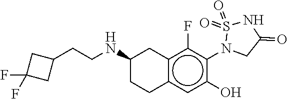 |
| 203 | 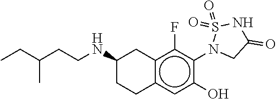 |
| 204 | 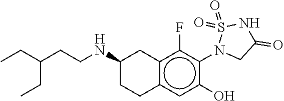 |
| 205 | 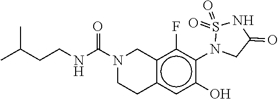 |
| 206 | 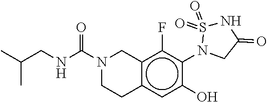 |
| 207 | 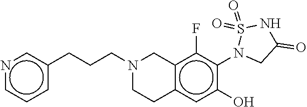 |
| 208 | 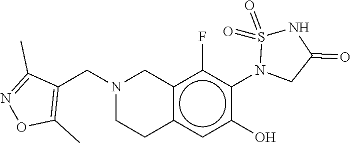 |
| 209 | 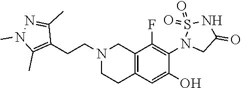 [SMILES: Cc1nn(C)c(C)c1CCN1CCc2cc(O)c(N3CC(=O)NS3(=O)=O)c(F)c2C1] |
| 210 | 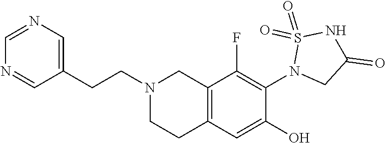 [SMILES: O=C1CN(c2c(O)cc3c(c2F)CN(CCc2cncnc2)CC3)S(=O)(=O)N1] |
| 211 | 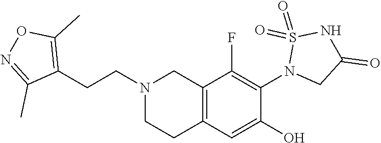 [SMILES: Cc1noc(C)c1CCN1CCc2cc(O)c(N3CC(=O)NS3(=O)=O)c(F)c2C1] |
| 212 | 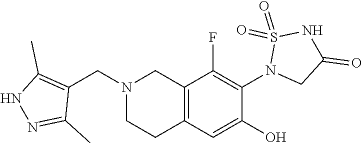 [SMILES: Cc1n[nH]c(C)c1CN1CCc2cc(O)c(N3CC(=O)NS3(=O)=O)c(F)c2C1] |
| 213 | 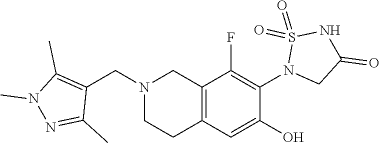 [SMILES: Cc1nn(C)c(C)c1CN1CCc2cc(O)c(N3CC(=O)NS3(=O)=O)c(F)c2C1] |
| 214 | 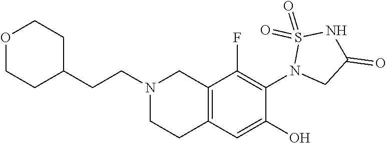 [SMILES: O=C1CN(c2c(O)cc3c(c2F)CN(CCC2CCOCC2)CC3)S(=O)(=O)N1] |
| 215 | 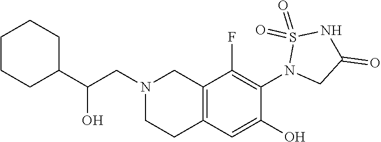 [SMILES: O=C1CN(c2c(O)cc3c(c2F)CN(CC(O)C2CCCCC2)CC3)S(=O)(=O)N1] |
| 216 | 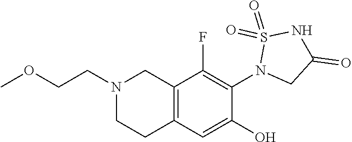 [SMILES: COCCN1CCc2cc(O)c(N3CC(=O)NS3(=O)=O)c(F)c2C1] |
| 217 | 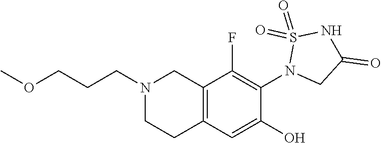 [SMILES: COCCCN1CCc2cc(O)c(N3CC(=O)NS3(=O)=O)c(F)c2C1] |
| 218 | 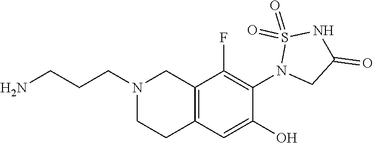 [SMILES: NCCCN1CCc2cc(O)c(N3CC(=O)NS3(=O)=O)c(F)c2C1] |
| 219 | 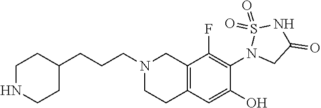 [SMILES: O=C1CN(c2c(O)cc3c(c2F)CN(CCCC2CCNCC2)CC3)S(=O)(=O)N1] |
| 220 | 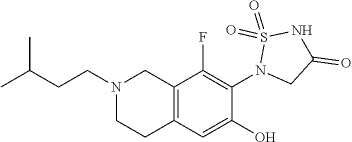 [SMILES: CC(C)CCN1CCc2cc(O)c(N3CC(=O)NS3(=O)=O)c(F)c2C1] |
| 221 | 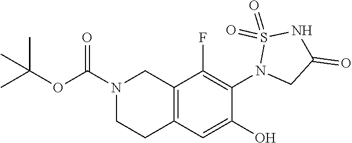 [SMILES: CC(C)(C)OC(=O)N1CCc2cc(O)c(N3CC(=O)NS3(=O)=O)c(F)c2C1] |
| 222 | 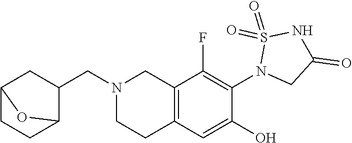 [SMILES: O=C1CN(c2c(O)cc3c(c2F)CN(CC2CC4CCC2O4)CC3)S(=O)(=O)N1] |
| 223 | 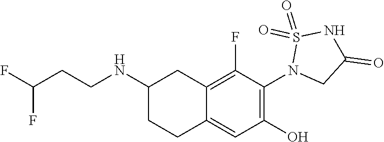 [SMILES: O=C1CN(c2c(O)cc3c(c2F)CC(NCCC(F)F)CC3)S(=O)(=O)N1] |
| 224 | 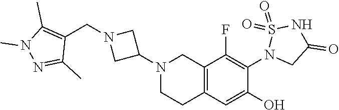 [SMILES: Cc1nn(C)c(C)c1CN1CC(N2CCc3cc(O)c(N4CC(=O)NS4(=O)=O)c(F)c3C2)C1] |
| 225 | 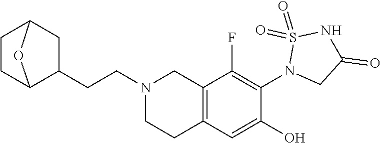 [SMILES: O=C1CN(c2c(O)cc3c(c2F)CN(CCC2CC4CCC2O4)CC3)S(=O)(=O)N1] |
| 226 | 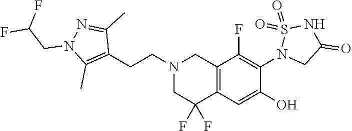 [SMILES: Cc1nn(CC(F)F)c(C)c1CCN1Cc2c(cc(O)c(N3CC(=O)NS3(=O)=O)c2F)C(F)(F)C1] |
| 227 | 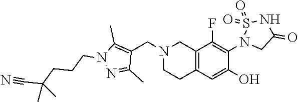 [SMILES: Cc1nn(CCCC(C)(C)C#N)c(C)c1CN1CCc2cc(O)c(N3CC(=O)NS3(=O)=O)c(F)c2C1] |
| 228 | 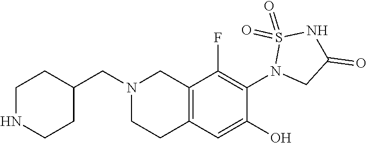 [SMILES: O=C1CN(c2c(O)cc3c(c2F)CN(CC2CCNCC2)CC3)S(=O)(=O)N1] |
| 229 | 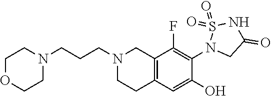 [SMILES: O=C1CN(c2c(O)cc3c(c2F)CN(CCCN2CCOCC2)CC3)S(=O)(=O)N1] |
| 230 | 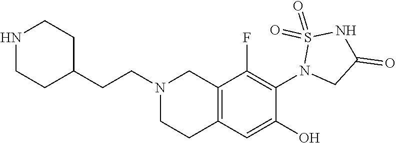 [SMILES: O=C1CN(c2c(O)cc3c(c2F)CN(CCC2CCNCC2)CC3)S(=O)(=O)N1] |
| 231 | 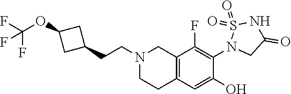 [SMILES: O=C1CN(c2c(O)cc3c(c2F)CN(CC[C@H]2C[C@@H](OC(F)(F)F)C2)CC3)S(=O)(=O)N1] |
| 232 | 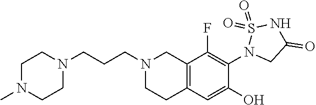 [SMILES: CN1CCN(CCCN2CCc3cc(O)c(N4CC(=O)NS4(=O)=O)c(F)c3C2)CC1] |
| 233 | 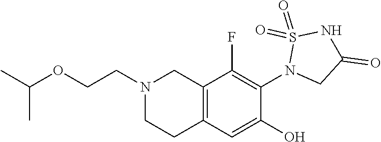 [SMILES: CC(C)OCCN1CCc2cc(O)c(N3CC(=O)NS3(=O)=O)c(F)c2C1] |
| 234 | 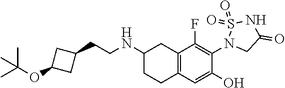 [SMILES: CC(C)(C)O[C@H]1C[C@@H](CCNC2CCc3cc(O)c(N4CC(=O)NS4(=O)=O)c(F)c3C2)C1] |
| 235 | 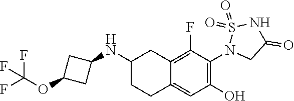 [SMILES: O=C1CN(c2c(O)cc3c(c2F)CC(N[C@H]2C[C@@H](OC(F)(F)F)C2)CC3)S(=O)(=O)N1] |
| 236 | 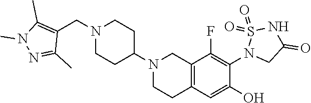 [SMILES: Cc1nn(C)c(C)c1CN1CCC(N2CCc3cc(O)c(N4CC(=O)NS4(=O)=O)c(F)c3C2)CC1] |
| 237 | 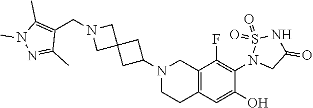 [SMILES: Cc1nn(C)c(C)c1CN1CC2(CC(N3CCc4cc(O)c(N5CC(=O)NS5(=O)=O)c(F)c4C3)C2)C1] |
| 238 | 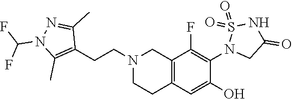 [SMILES: Cc1nn(C(F)F)c(C)c1CCN1CCc2cc(O)c(N3CC(=O)NS3(=O)=O)c(F)c2C1] |
| 239 | 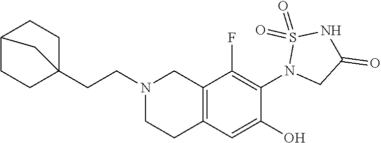 [SMILES: O=C1CN(c2c(O)cc3c(c2F)CN(CCC24CCC(CC2)C4)CC3)S(=O)(=O)N1] |
| 240 | 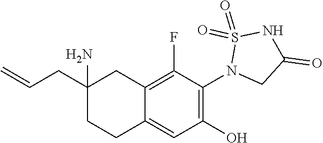 [SMILES: C=CCC1(N)CCc2cc(O)c(N3CC(=O)NS3(=O)=O)c(F)c2C1] |
| 241 | 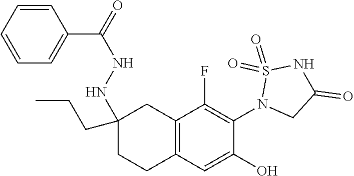 [SMILES: CCCC1(NNC(=O)c2ccccc2)CCc2cc(O)c(N3CC(=O)NS3(=O)=O)c(F)c2C1] |
| 242 | 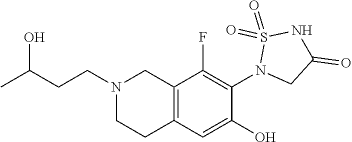 [SMILES: CC(O)CCN1CCc2cc(O)c(N3CC(=O)NS3(=O)=O)c(F)c2C1] |
| 243 | 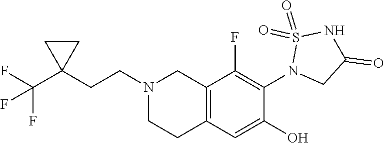 [SMILES: O=C1CN(c2c(O)cc3c(c2F)CN(CCC2(C(F)(F)F)CC2)CC3)S(=O)(=O)N1] |
| 244 | 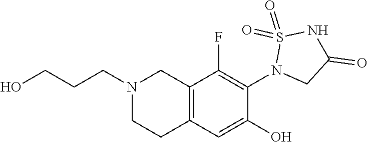 [SMILES: O=C1CN(c2c(O)cc3c(c2F)CN(CCCO)CC3)S(=O)(=O)N1] |
| 245 | 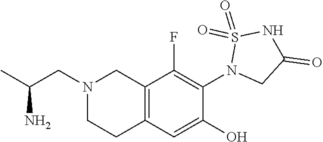 [SMILES: C[C@H](N)CN1CCc2cc(O)c(N3CC(=O)NS3(=O)=O)c(F)c2C1] |
| 246 | 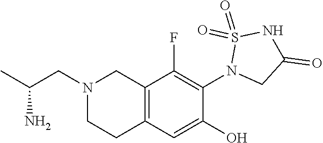 [SMILES: C[C@@H](N)CN1CCc2cc(O)c(N3CC(=O)NS3(=O)=O)c(F)c2C1] |
| 247 | 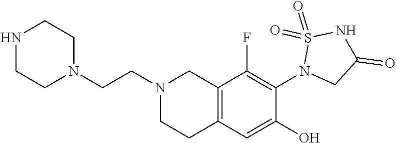 [SMILES: O=C1CN(c2c(O)cc3c(c2F)CN(CCN2CCNCC2)CC3)S(=O)(=O)N1] |
| 248 | 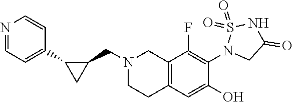 [SMILES: O=C1CN(c2c(O)cc3c(c2F)CN(C[C@H]2C[C@@H]2c2ccncc2)CC3)S(=O)(=O)N1] |
| 249 | 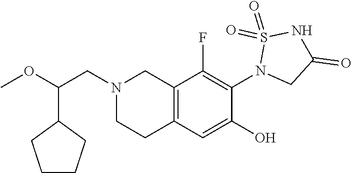 [SMILES: COC(CN1CCc2cc(O)c(N3CC(=O)NS3(=O)=O)c(F)c2C1)C1CCCC1] |
| 250 | 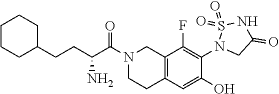 [SMILES: N[C@H](CCC1CCCCC1)C(=O)N1CCc2cc(O)c(N3CC(=O)NS3(=O)=O)c(F)c2C1] |
| 251 | 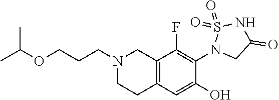 [SMILES: CC(C)OCCCN1CCc2cc(O)c(N3CC(=O)NS3(=O)=O)c(F)c2C1] |
| 252 | 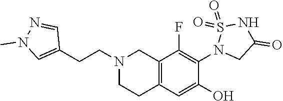 [SMILES: Cn1cc(CCN2CCc3cc(O)c(N4CC(=O)NS4(=O)=O)c(F)c3C2)cn1] |
| 253 | 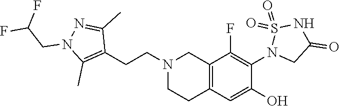 [SMILES: Cc1nn(CC(F)F)c(C)c1CCN1CCc2cc(O)c(N3CC(=O)NS3(=O)=O)c(F)c2C1] |
| 254 | 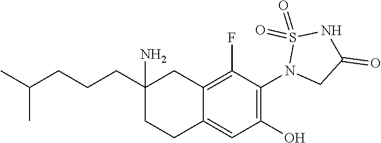 [SMILES: CC(C)CCCC1(N)CCc2cc(O)c(N3CC(=O)NS3(=O)=O)c(F)c2C1] |
| 255 | 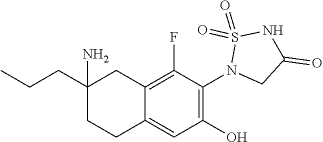 [SMILES: CCCC1(N)CCc2cc(O)c(N3CC(=O)NS3(=O)=O)c(F)c2C1] |
| 256 | 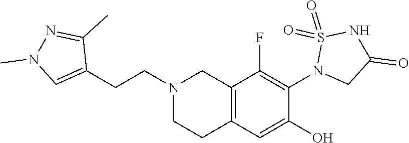 [SMILES: Cc1nn(C)cc1CCN1CCc2cc(O)c(N3CC(=O)NS3(=O)=O)c(F)c2C1] |
| 257 | 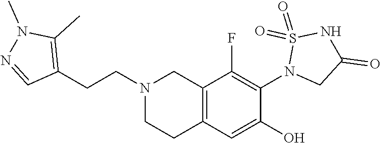 [SMILES: Cc1c(CCN2CCc3cc(O)c(N4CC(=O)NS4(=O)=O)c(F)c3C2)cnn1C] |
| 258 | 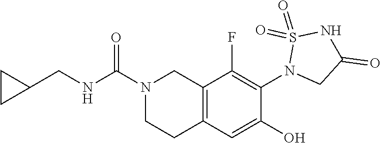 [SMILES: O=C1CN(c2c(O)cc3c(c2F)CN(C(=O)NCC2CC2)CC3)S(=O)(=O)N1] |
| 259 | 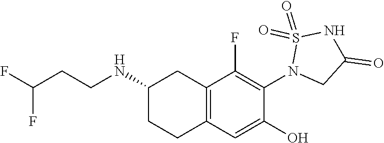 [SMILES: O=C1CN(c2c(O)cc3c(c2F)C[C@@H](NCCC(F)F)CC3)S(=O)(=O)N1] |
| 260 | 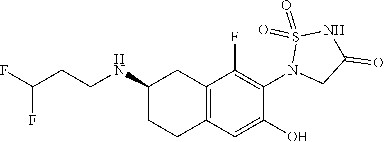 [SMILES: O=C1CN(c2c(O)cc3c(c2F)C[C@H](NCCC(F)F)CC3)S(=O)(=O)N1] |
| 261 | 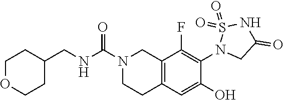 [SMILES: O=C1CN(c2c(O)cc3c(c2F)CN(C(=O)NCC2CCOCC2)CC3)S(=O)(=O)N1] |
| 262 | 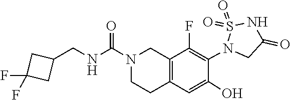 [SMILES: O=C1CN(c2c(O)cc3c(c2F)CN(C(=O)NCC2CC(F)(F)C2)CC3)S(=O)(=O)N1] |
| 263 | 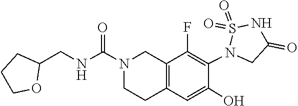 [SMILES: O=C1CN(c2c(O)cc3c(c2F)CN(C(=O)NCC2CCCO2)CC3)S(=O)(=O)N1] |
| 264 | 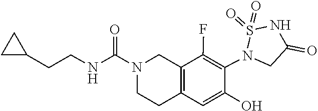 [SMILES: O=C1CN(c2c(O)cc3c(c2F)CN(C(=O)NCCC2CC2)CC3)S(=O)(=O)N1] |
| 265 | 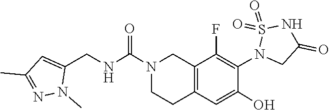 [SMILES: Cc1cc(CNC(=O)N2CCc3cc(O)c(N4CC(=O)NS4(=O)=O)c(F)c3C2)n(C)n1] |
| 266 | 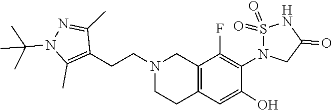 [SMILES: Cc1nn(C(C)(C)C)c(C)c1CCN1CCc2cc(O)c(N3CC(=O)NS3(=O)=O)c(F)c2C1] |
| 267 | 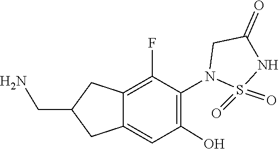 [SMILES: NCC1Cc2cc(O)c(N3CC(=O)NS3(=O)=O)c(F)c2C1] |
| 268 | 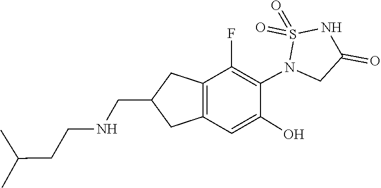 [SMILES: CC(C)CCNCC1Cc2cc(O)c(N3CC(=O)NS3(=O)=O)c(F)c2C1] |
| 269 | 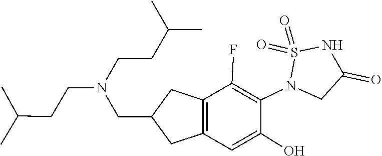 [SMILES: CC(C)CCN(CCC(C)C)CC1Cc2cc(O)c(N3CC(=O)NS3(=O)=O)c(F)c2C1] |
| 270 | 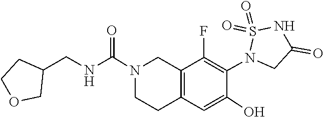 [SMILES: O=C1CN(c2c(O)cc3c(c2F)CN(C(=O)NCC2CCOC2)CC3)S(=O)(=O)N1] |
| 271 | 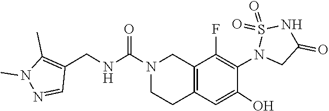 [SMILES: Cc1c(CNC(=O)N2CCc3cc(O)c(N4CC(=O)NS4(=O)=O)c(F)c3C2)cnn1C] |
| 272 | 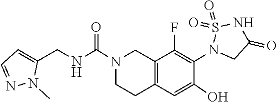 [SMILES: Cn1nccc1CNC(=O)N1CCc2cc(O)c(N3CC(=O)NS3(=O)=O)c(F)c2C1] |
| 273 | 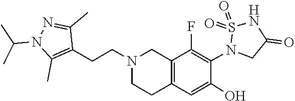 [SMILES: Cc1nn(C(C)C)c(C)c1CCN1CCc2cc(O)c(N3CC(=O)NS3(=O)=O)c(F)c2C1] |
| 274 | 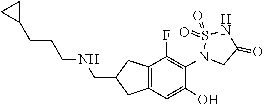 [SMILES: O=C1CN(c2c(O)cc3c(c2F)CC(CNCCCC2CC2)C3)S(=O)(=O)N1] |
| 275 | 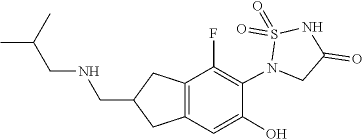 [SMILES: CC(C)CNCC1Cc2cc(O)c(N3CC(=O)NS3(=O)=O)c(F)c2C1] |
| 276 | 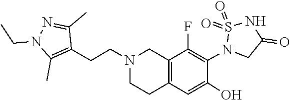 [SMILES: CCn1nc(C)c(CCN2CCc3cc(O)c(N4CC(=O)NS4(=O)=O)c(F)c3C2)c1C] |
| 277 | 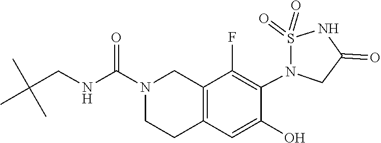 [SMILES: CC(C)(C)CNC(=O)N1CCc2cc(O)c(N3CC(=O)NS3(=O)=O)c(F)c2C1] |
| 278 | 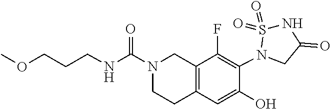 [SMILES: COCCCNC(=O)N1CCc2cc(O)c(N3CC(=O)NS3(=O)=O)c(F)c2C1] |
| 279 | 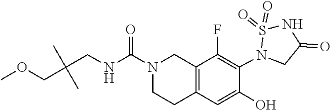 [SMILES: COCC(C)(C)CNC(=O)N1CCc2cc(O)c(N3CC(=O)NS3(=O)=O)c(F)c2C1] |
| 280 | 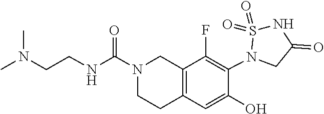 [SMILES: CN(C)CCNC(=O)N1CCc2cc(O)c(N3CC(=O)NS3(=O)=O)c(F)c2C1] |
| 281 | 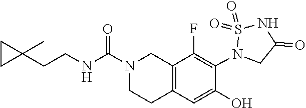 [SMILES: CC1(CCNC(=O)N2CCc3cc(O)c(N4CC(=O)NS4(=O)=O)c(F)c3C2)CC1] |
| 282 | 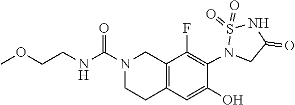 [SMILES: COCCNC(=O)N1CCc2cc(O)c(N3CC(=O)NS3(=O)=O)c(F)c2C1] |
| 283 | 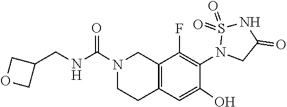 [SMILES: O=C1CN(c2c(O)cc3c(c2F)CN(C(=O)NCC2COC2)CC3)S(=O)(=O)N1] |
| 284 | 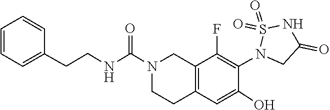 [SMILES: O=C1CN(c2c(O)cc3c(c2F)CN(C(=O)NCCc2ccccc2)CC3)S(=O)(=O)N1] |
| 285 | 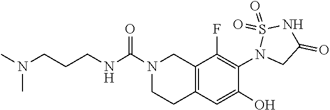 [SMILES: CN(C)CCCNC(=O)N1CCc2cc(O)c(N3CC(=O)NS3(=O)=O)c(F)c2C1] |
| 286 | 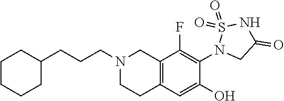 [SMILES: O=C1CN(c2c(O)cc3c(c2F)CN(CCCC2CCCCC2)CC3)S(=O)(=O)N1] |
| 287 | 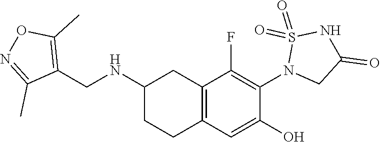 [SMILES: Cc1noc(C)c1CNC1CCc2cc(O)c(N3CC(=O)NS3(=O)=O)c(F)c2C1] |
| 288 | 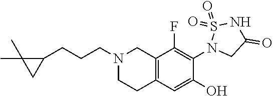 [SMILES: CC1(C)CC1CCCN1CCc2cc(O)c(N3CC(=O)NS3(=O)=O)c(F)c2C1] |
| 289 | 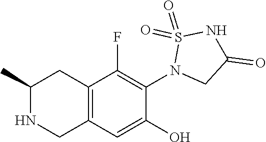 [SMILES: C[C@H]1Cc2c(cc(O)c(N3CC(=O)NS3(=O)=O)c2F)CN1] |
| 290 | 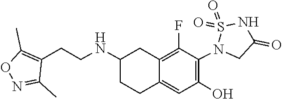 [SMILES: Cc1noc(C)c1CCNC1CCc2cc(O)c(N3CC(=O)NS3(=O)=O)c(F)c2C1] |
| 291 | 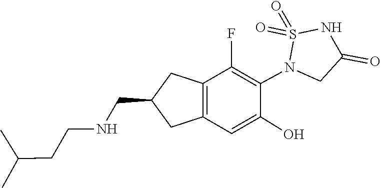 [SMILES: CC(C)CCNC[C@@H]1Cc2cc(O)c(N3CC(=O)NS3(=O)=O)c(F)c2C1] |
| 292 | 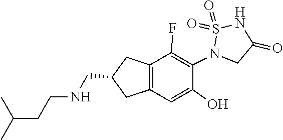 [SMILES: CC(C)CCNC[C@H]1Cc2cc(O)c(N3CC(=O)NS3(=O)=O)c(F)c2C1] |
| 293 | 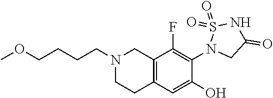 [SMILES: COCCCCN1CCc2cc(O)c(N3CC(=O)NS3(=O)=O)c(F)c2C1] |
| 294 | 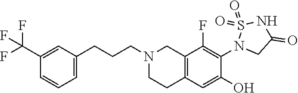 [SMILES: O=C1CN(c2c(O)cc3c(c2F)CN(CCCc2cccc(C(F)(F)F)c2)CC3)S(=O)(=O)N1] |
| 295 | 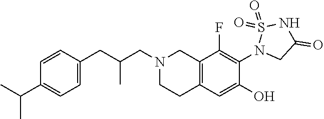 [SMILES: CC(Cc1ccc(C(C)C)cc1)CN1CCc2cc(O)c(N3CC(=O)NS3(=O)=O)c(F)c2C1] |
| 296 | 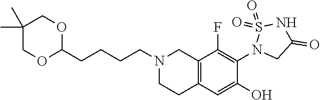 [SMILES: CC1(C)COC(CCCCN2CCc3cc(O)c(N4CC(=O)NS4(=O)=O)c(F)c3C2)OC1] |
| 297 | 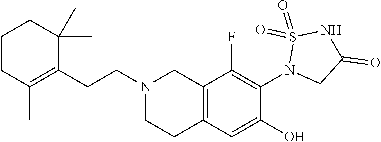 [SMILES: CC1=C(CCN2CCc3cc(O)c(N4CC(=O)NS4(=O)=O)c(F)c3C2)C(C)(C)CCC1] |
| 298 | 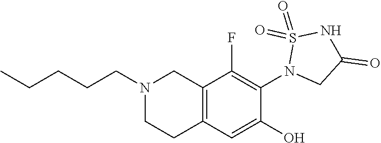 [SMILES: CCCCCN1CCc2cc(O)c(N3CC(=O)NS3(=O)=O)c(F)c2C1] |
| 299 | 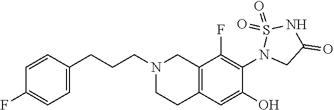 [SMILES: O=C1CN(c2c(O)cc3c(c2F)CN(CCCc2ccc(F)cc2)CC3)S(=O)(=O)N1] |
| 300 | 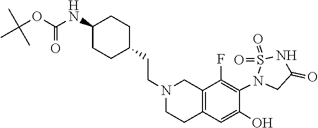 [SMILES: CC(C)(C)OC(=O)N[C@H]1CC[C@H](CCN2CCc3cc(O)c(N4CC(=O)NS4(=O)=O)c(F)c3C2)CC1] |
| 301 | 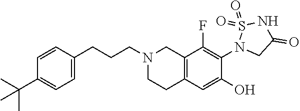 [SMILES: CC(C)(C)c1ccc(CCCN2CCc3cc(O)c(N4CC(=O)NS4(=O)=O)c(F)c3C2)cc1] |
| 302 | 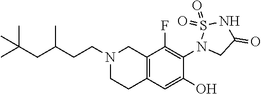 [SMILES: CC(CCN1CCc2cc(O)c(N3CC(=O)NS3(=O)=O)c(F)c2C1)CC(C)(C)C] |
| 303 | 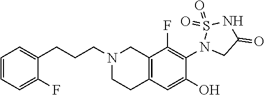 [SMILES: O=C1CN(c2c(O)cc3c(c2F)CN(CCCc2ccccc2F)CC3)S(=O)(=O)N1] |
| 304 | 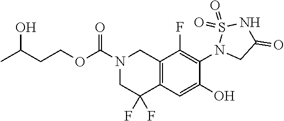 [SMILES: CC(O)CCOC(=O)N1Cc2c(cc(O)c(N3CC(=O)NS3(=O)=O)c2F)C(F)(F)C1] |
| 305 | 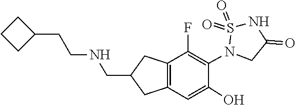 [SMILES: O=C1CN(c2c(O)cc3c(c2F)CC(CNCCC2CCC2)C3)S(=O)(=O)N1] |
| 306 | 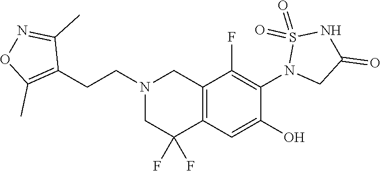 [SMILES: Cc1noc(C)c1CCN1Cc2c(cc(O)c(N3CC(=O)NS3(=O)=O)c2F)C(F)(F)C1] |
| 307 | 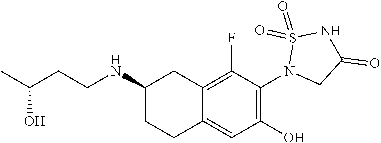 [SMILES: C[C@@H](O)CCN[C@@H]1CCc2cc(O)c(N3CC(=O)NS3(=O)=O)c(F)c2C1] |
| 308 | 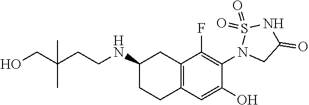 [SMILES: CC(C)(CO)CCN[C@@H]1CCc2cc(O)c(N3CC(=O)NS3(=O)=O)c(F)c2C1] |
| 309 | 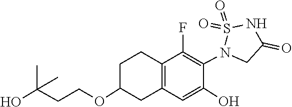 [SMILES: CC(C)(O)CCOC1CCc2c(cc(O)c(N3CC(=O)NS3(=O)=O)c2F)C1] |
| 310 | 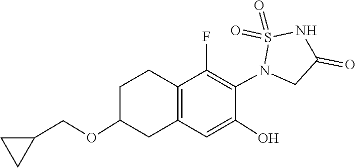 [SMILES: O=C1CN(c2c(O)cc3c(c2F)CCC(OCC2CC2)C3)S(=O)(=O)N1] |
| 311 | 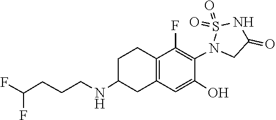 [SMILES: O=C1CN(c2c(O)cc3c(c2F)CCC(NCCCC(F)F)C3)S(=O)(=O)N1] |
| 312 | 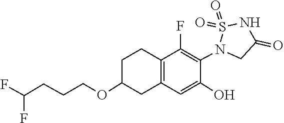 [SMILES: O=C1CN(c2c(O)cc3c(c2F)CCC(OCCCC(F)F)C3)S(=O)(=O)N1] |
| 313 | 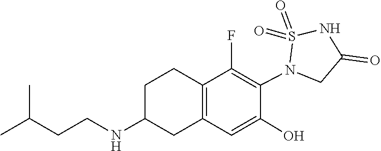 [SMILES: CC(C)CCNC1CCc2c(cc(O)c(N3CC(=O)NS3(=O)=O)c2F)C1] |
| 314 | 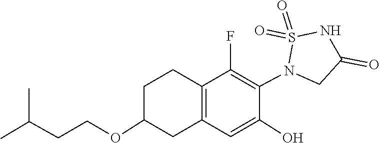 [SMILES: CC(C)CCOC1CCc2c(cc(O)c(N3CC(=O)NS3(=O)=O)c2F)C1] |
| 315 | 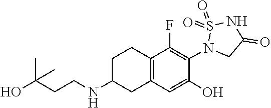 [SMILES: CC(C)(O)CCNC1CCc2c(cc(O)c(N3CC(=O)NS3(=O)=O)c2F)C1] |
| 316 | 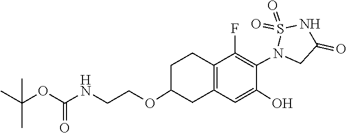 [SMILES: CC(C)(C)OC(=O)NCCOC1CCc2c(cc(O)c(N3CC(=O)NS3(=O)=O)c2F)C1] |
| 317 | 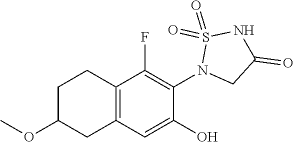 [SMILES: COC1CCc2c(cc(O)c(N3CC(=O)NS3(=O)=O)c2F)C1] |
| 318 | 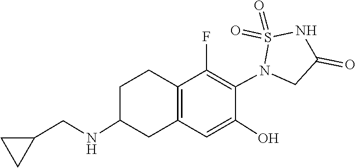 [SMILES: O=C1CN(c2c(O)cc3c(c2F)CCC(NCC2CC2)C3)S(=O)(=O)N1] |
| 319 | 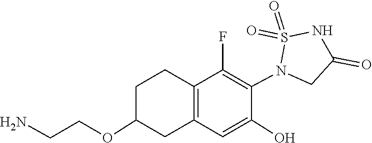 [SMILES: NCCOC1CCc2c(cc(O)c(N3CC(=O)NS3(=O)=O)c2F)C1] |
| 320 | 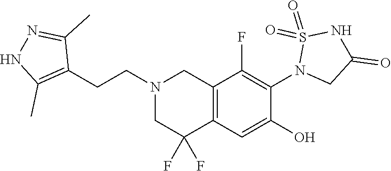 [SMILES: Cc1n[nH]c(C)c1CCN1Cc2c(cc(O)c(N3CC(=O)NS3(=O)=O)c2F)C(F)(F)C1] |
| 321 | 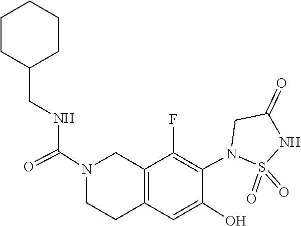 [SMILES: O=C1CN(c2c(O)cc3c(c2F)CN(C(=O)NCC2CCCCC2)CC3)S(=O)(=O)N1] |
| 322 | 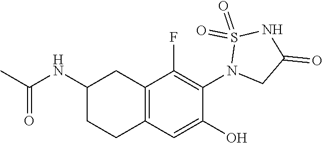 [SMILES: CC(=O)NC1CCc2cc(O)c(N3CC(=O)NS3(=O)=O)c(F)c2C1] |
| 323 | 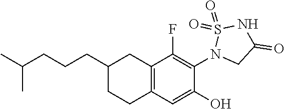 [SMILES: CC(C)CCCC1CCc2cc(O)c(N3CC(=O)NS3(=O)=O)c(F)c2C1] |
| 324 | 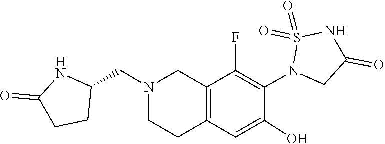 [SMILES: O=C1CC[C@@H](CN2CCc3cc(O)c(N4CC(=O)NS4(=O)=O)c(F)c3C2)N1] |
| 325 | 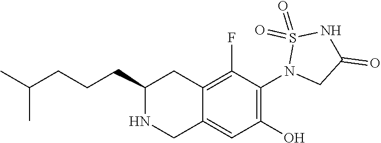 [SMILES: CC(C)CCC[C@H]1Cc2c(cc(O)c(N3CC(=O)NS3(=O)=O)c2F)CN1] |
| 326 | 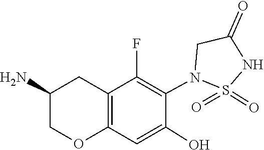 [SMILES: N[C@@H]1COc2cc(O)c(N3CC(=O)NS3(=O)=O)c(F)c2C1] |
| 327 | 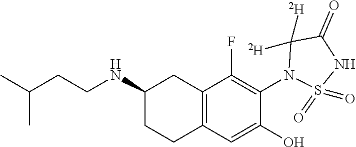 [SMILES: [2H]C1([2H])C(=O)NS(=O)(=O)N1c1c(O)cc2c(c1F)C[C@H](NCCC(C)C)CC2] |
| 328 | 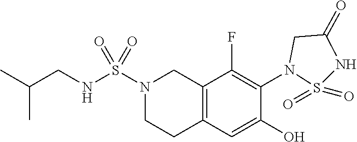 [SMILES: CC(C)CNS(=O)(=O)N1CCc2cc(O)c(N3CC(=O)NS3(=O)=O)c(F)c2C1] |
| 329 | 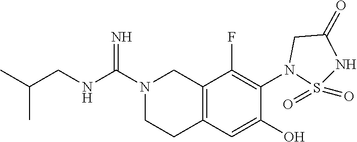 [SMILES: CC(C)CNC(=N)N1CCc2cc(O)c(N3CC(=O)NS3(=O)=O)c(F)c2C1] |
| 330 | 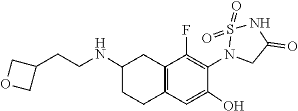 [SMILES: O=C1CN(c2c(O)cc3c(c2F)CC(NCCC2COC2)CC3)S(=O)(=O)N1] |
| 331 | 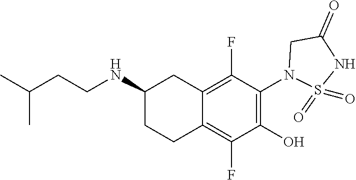 [SMILES: CC(C)CCN[C@@H]1CCc2c(F)c(O)c(N3CC(=O)NS3(=O)=O)c(F)c2C1] |
| 332 | 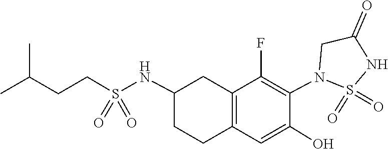 [SMILES: CC(C)CCS(=O)(=O)NC1CCc2cc(O)c(N3CC(=O)NS3(=O)=O)c(F)c2C1] |
| 333 | 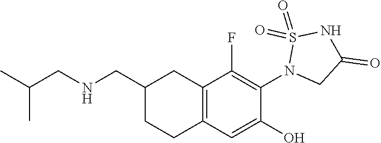 [SMILES: CC(C)CNCC1CCc2cc(O)c(N3CC(=O)NS3(=O)=O)c(F)c2C1] |
| 334 | 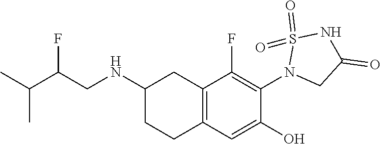 [SMILES: CC(C)C(F)CNC1CCc2cc(O)c(N3CC(=O)NS3(=O)=O)c(F)c2C1] |
| 335 | 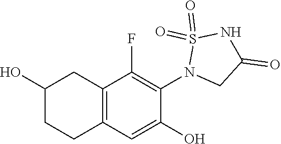 [SMILES: O=C1CN(c2c(O)cc3c(c2F)CC(O)CC3)S(=O)(=O)N1] |
| 336 | 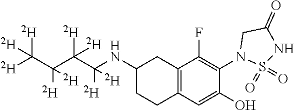 [SMILES: [2H]C([2H])([2H])C([2H])([2H])C([2H])([2H])C([2H])([2H])NC1CCc2cc(O)c(N3CC(=O)NS3(=O)=O)c(F)c2C1] |
| 337 | 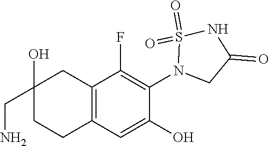 [SMILES: NCC1(O)CCc2cc(O)c(N3CC(=O)NS3(=O)=O)c(F)c2C1] |
| 338 | 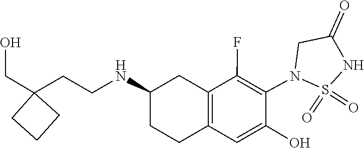 [SMILES: O=C1CN(c2c(O)cc3c(c2F)C[C@H](NCCC2(CO)CCC2)CC3)S(=O)(=O)N1] |
| 339 | 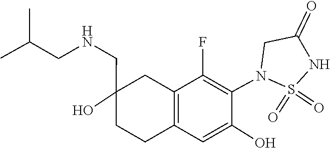 [SMILES: CC(C)CNCC1(O)CCc2cc(O)c(N3CC(=O)NS3(=O)=O)c(F)c2C1] |
| 340 | 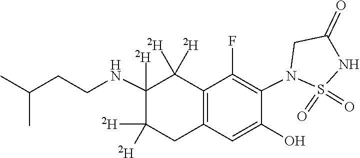 [SMILES: [2H]C1([2H])Cc2cc(O)c(N3CC(=O)NS3(=O)=O)c(F)c2C([2H])([2H])C1([2H])NCCC(C)C] |
| 341 | 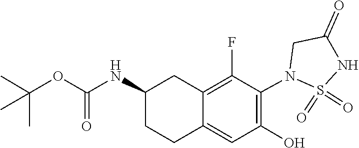 [SMILES: CC(C)(C)OC(=O)N[C@@H]1CCc2cc(O)c(N3CC(=O)NS3(=O)=O)c(F)c2C1] |
| 342 | 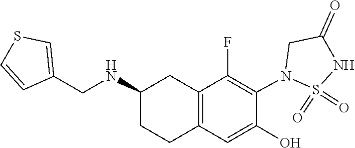 [SMILES: O=C1CN(c2c(O)cc3c(c2F)C[C@H](NCc2ccsc2)CC3)S(=O)(=O)N1] |
| 343 | 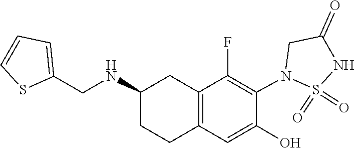 [SMILES: O=C1CN(c2c(O)cc3c(c2F)C[C@H](NCc2cccs2)CC3)S(=O)(=O)N1] |
| 344 | 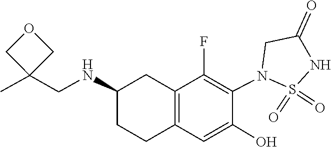 [SMILES: CC1(CN[C@@H]2CCc3cc(O)c(N4CC(=O)NS4(=O)=O)c(F)c3C2)COC1] |
| 345 | 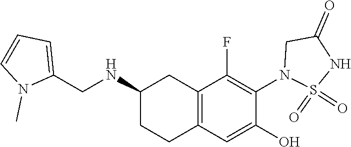 [SMILES: Cn1cccc1CN[C@@H]1CCc2cc(O)c(N3CC(=O)NS3(=O)=O)c(F)c2C1] |
| 346 | 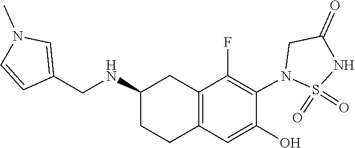 [SMILES: Cn1ccc(CN[C@@H]2CCc3cc(O)c(N4CC(=O)NS4(=O)=O)c(F)c3C2)c1] |
| 347 | 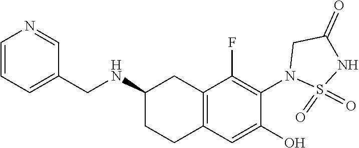 [SMILES: O=C1CN(c2c(O)cc3c(c2F)C[C@H](NCc2cccnc2)CC3)S(=O)(=O)N1] |
| 348 | 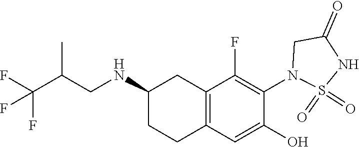 [SMILES: CC(CN[C@@H]1CCc2cc(O)c(N3CC(=O)NS3(=O)=O)c(F)c2C1)C(F)(F)F] |
| 349 | 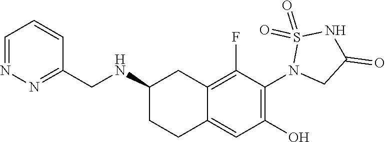 [SMILES: O=C1CN(c2c(O)cc3c(c2F)C[C@H](NCc2cccnn2)CC3)S(=O)(=O)N1] |
| 350 | 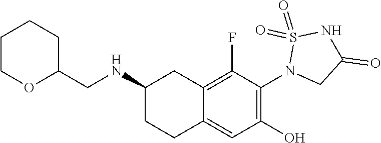 [SMILES: O=C1CN(c2c(O)cc3c(c2F)C[C@H](NCC2CCCCO2)CC3)S(=O)(=O)N1] |
| 351 | 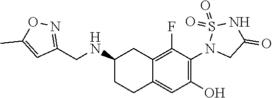 [SMILES: Cc1cc(CN[C@@H]2CCc3cc(O)c(N4CC(=O)NS4(=O)=O)c(F)c3C2)no1] |
| 352 | 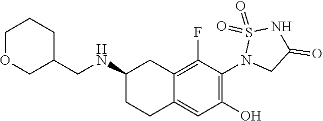 [SMILES: O=C1CN(c2c(O)cc3c(c2F)C[C@H](NCC2CCCOC2)CC3)S(=O)(=O)N1] |
| 353 | 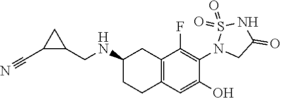 [SMILES: N#CC1CC1CN[C@@H]1CCc2cc(O)c(N3CC(=O)NS3(=O)=O)c(F)c2C1] |
| 354 | 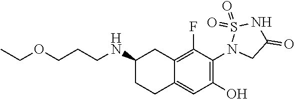 [SMILES: CCOCCCN[C@@H]1CCc2cc(O)c(N3CC(=O)NS3(=O)=O)c(F)c2C1] |
| 355 | 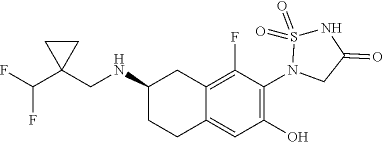 [SMILES: O=C1CN(c2c(O)cc3c(c2F)C[C@H](NCC2(C(F)F)CC2)CC3)S(=O)(=O)N1] |
| 356 | 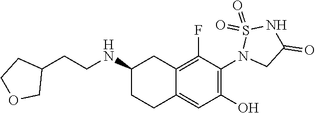 [SMILES: O=C1CN(c2c(O)cc3c(c2F)C[C@H](NCCC2CCOC2)CC3)S(=O)(=O)N1] |
| 357 | 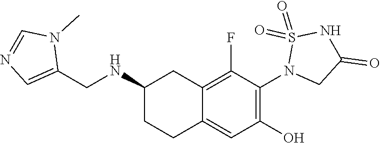 [SMILES: Cn1cncc1CN[C@@H]1CCc2cc(O)c(N3CC(=O)NS3(=O)=O)c(F)c2C1] |
| 358 | 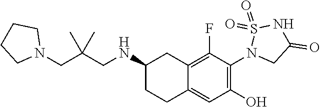 [SMILES: CC(C)(CN[C@@H]1CCc2cc(O)c(N3CC(=O)NS3(=O)=O)c(F)c2C1)CN1CCCC1] |
| 359 | 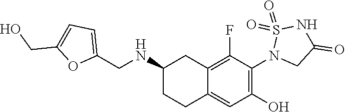 [SMILES: O=C1CN(c2c(O)cc3c(c2F)C[C@H](NCc2ccc(CO)o2)CC3)S(=O)(=O)N1] |
| 360 | 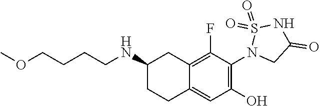 [SMILES: COCCCCN[C@@H]1CCc2cc(O)c(N3CC(=O)NS3(=O)=O)c(F)c2C1] |
| 361 | 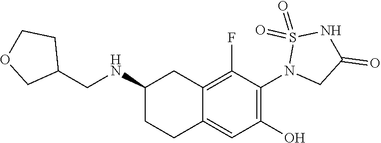 [SMILES: O=C1CN(c2c(O)cc3c(c2F)C[C@H](NCC2CCOC2)CC3)S(=O)(=O)N1] |
| 362 | 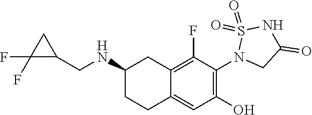 [SMILES: O=C1CN(c2c(O)cc3c(c2F)C[C@H](NCC2CC2(F)F)CC3)S(=O)(=O)N1] |
| 363 | 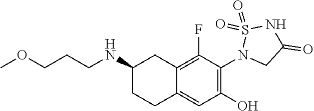 [SMILES: COCCCN[C@@H]1CCc2cc(O)c(N3CC(=O)NS3(=O)=O)c(F)c2C1] |
| 364 | 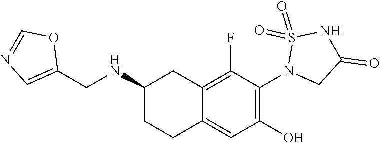 [SMILES: O=C1CN(c2c(O)cc3c(c2F)C[C@H](NCc2cnco2)CC3)S(=O)(=O)N1] |
| 365 | 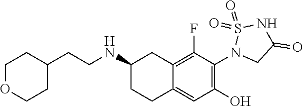 [SMILES: O=C1CN(c2c(O)cc3c(c2F)C[C@H](NCCC2CCOCC2)CC3)S(=O)(=O)N1] |
| 366 | 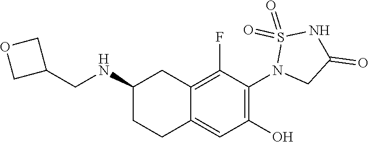 [SMILES: O=C1CN(c2c(O)cc3c(c2F)C[C@H](NCC2COC2)CC3)S(=O)(=O)N1] |
| 367 | 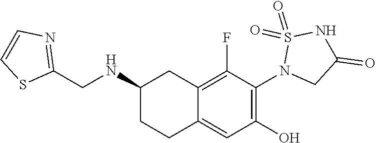 [SMILES: O=C1CN(c2c(O)cc3c(c2F)C[C@H](NCc2nccs2)CC3)S(=O)(=O)N1] |
| 368 | 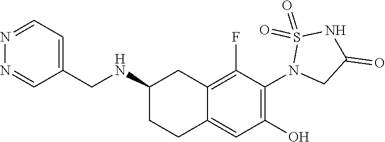 [SMILES: O=C1CN(c2c(O)cc3c(c2F)C[C@H](NCc2ccnnc2)CC3)S(=O)(=O)N1] |
| 369 | 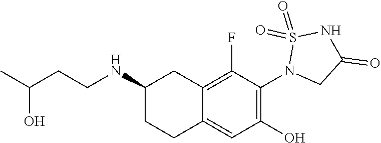 [SMILES: CC(O)CCN[C@@H]1CCc2cc(O)c(N3CC(=O)NS3(=O)=O)c(F)c2C1] |
| 370 | 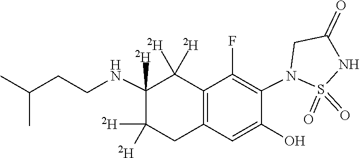 [SMILES: [2H]C1([2H])Cc2cc(O)c(N3CC(=O)NS3(=O)=O)c(F)c2C([2H])([2H])[C@@]1([2H])NCCC(C)C] |
| 371 | 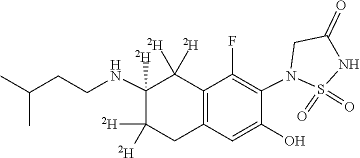 [SMILES: [2H]C1([2H])Cc2cc(O)c(N3CC(=O)NS3(=O)=O)c(F)c2C([2H])([2H])[C@]1([2H])NCCC(C)C] |
| 372 | 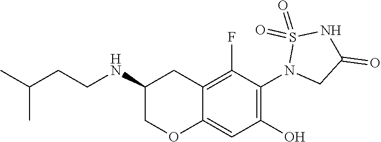 [SMILES: CC(C)CCN[C@@H]1COc2cc(O)c(N3CC(=O)NS3(=O)=O)c(F)c2C1] |
| 373 | 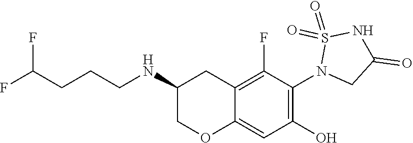 [SMILES: O=C1CN(c2c(O)cc3c(c2F)C[C@H](NCCCC(F)F)CO3)S(=O)(=O)N1] |
| 374 | 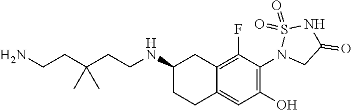 [SMILES: CC(C)(CCN)CCN[C@@H]1CCc2cc(O)c(N3CC(=O)NS3(=O)=O)c(F)c2C1] |
| 375 | 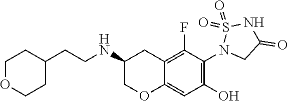 [SMILES: O=C1CN(c2c(O)cc3c(c2F)C[C@H](NCCC2CCOCC2)CO3)S(=O)(=O)N1] |
| 376 | 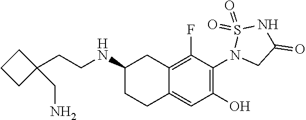 [SMILES: NCC1(CCN[C@@H]2CCc3cc(O)c(N4CC(=O)NS4(=O)=O)c(F)c3C2)CCC1] |
| 377 | 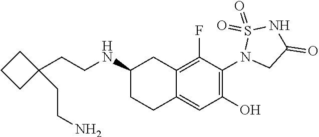 [SMILES: NCCC1(CCN[C@@H]2CCc3cc(O)c(N4CC(=O)NS4(=O)=O)c(F)c3C2)CCC1] |
| 378 | 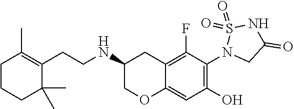 [SMILES: CC1=C(CCN[C@@H]2COc3cc(O)c(N4CC(=O)NS4(=O)=O)c(F)c3C2)C(C)(C)CCC1] |
| 379 | 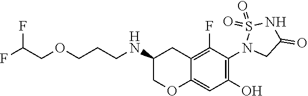 [SMILES: O=C1CN(c2c(O)cc3c(c2F)C[C@H](NCCCOCC(F)F)CO3)S(=O)(=O)N1] |
| 380 | 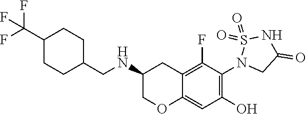 [SMILES: O=C1CN(c2c(O)cc3c(c2F)C[C@H](NCC2CCC(C(F)(F)F)CC2)CO3)S(=O)(=O)N1] |
| 381 | 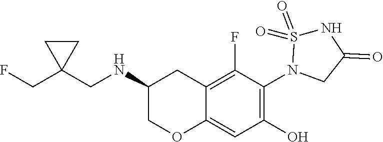 [SMILES: O=C1CN(c2c(O)cc3c(c2F)C[C@H](NCC2(CF)CC2)CO3)S(=O)(=O)N1] |
| 382 | 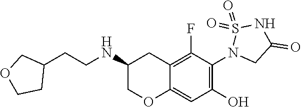 [SMILES: O=C1CN(c2c(O)cc3c(c2F)C[C@H](NCCC2CCOC2)CO3)S(=O)(=O)N1] |
| 383 | 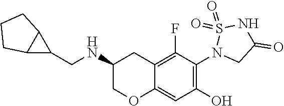 [SMILES: O=C1CN(c2c(O)cc3c(c2F)C[C@H](NCC2C4CCCC42)CO3)S(=O)(=O)N1] |
| 384 | 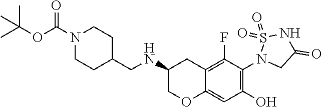 [SMILES: CC(C)(C)OC(=O)N1CCC(CN[C@@H]2COc3cc(O)c(N4CC(=O)NS4(=O)=O)c(F)c3C2)CC1] |
| 385 | 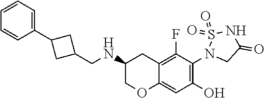 [SMILES: O=C1CN(c2c(O)cc3c(c2F)C[C@H](NCC2CC(c4ccccc4)C2)CO3)S(=O)(=O)N1] |
| 386 | 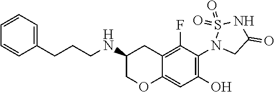 [SMILES: O=C1CN(c2c(O)cc3c(c2F)C[C@H](NCCCc2ccccc2)CO3)S(=O)(=O)N1] |
| 387 | 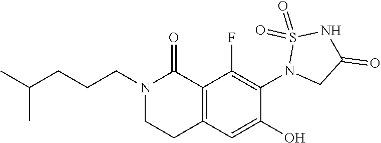 [SMILES: CC(C)CCCN1CCc2cc(O)c(N3CC(=O)NS3(=O)=O)c(F)c2C1=O] |
| 388 | 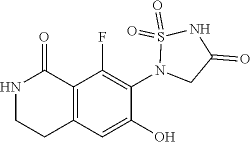 [SMILES: O=C1CN(c2c(O)cc3c(c2F)C(=O)NCC3)S(=O)(=O)N1] |
| 389 | 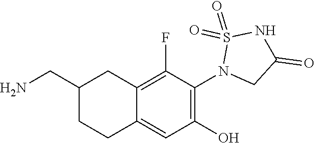 [SMILES: NCC1CCc2cc(O)c(N3CC(=O)NS3(=O)=O)c(F)c2C1] |
| 390 | 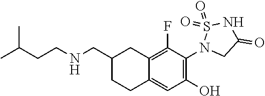 [SMILES: CC(C)CCNCC1CCc2cc(O)c(N3CC(=O)NS3(=O)=O)c(F)c2C1] |
| 391 | 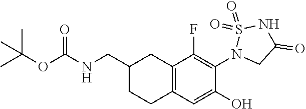 [SMILES: CC(C)(C)OC(=O)NCC1CCc2cc(O)c(N3CC(=O)NS3(=O)=O)c(F)c2C1] |
| 392 | 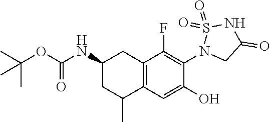 [SMILES: CC1C[C@@H](NC(=O)OC(C)(C)C)Cc2c1cc(O)c(N1CC(=O)NS1(=O)=O)c2F] |
| 393 | 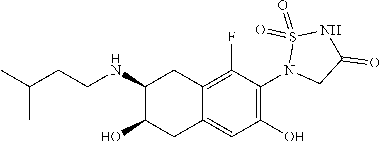 [SMILES: CC(C)CCN[C@H]1Cc2c(cc(O)c(N3CC(=O)NS3(=O)=O)c2F)C[C@H]1O] |
| 394 | 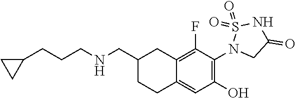 [SMILES: O=C1CN(c2c(O)cc3c(c2F)CC(CNCCCC2CC2)CC3)S(=O)(=O)N1] |
| 395 | 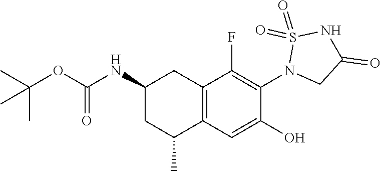 [SMILES: C[C@@H]1C[C@@H](NC(=O)OC(C)(C)C)Cc2c1cc(O)c(N1CC(=O)NS1(=O)=O)c2F] |
| 396 | 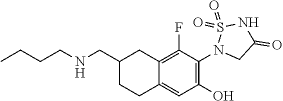 [SMILES: CCCCNCC1CCc2cc(O)c(N3CC(=O)NS3(=O)=O)c(F)c2C1] |
| 397 | 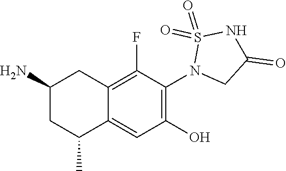 [SMILES: C[C@@H]1C[C@@H](N)Cc2c1cc(O)c(N1CC(=O)NS1(=O)=O)c2F] |
| 398 | 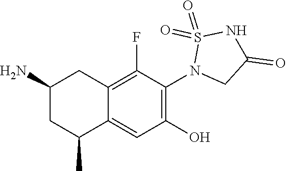 [SMILES: C[C@H]1C[C@@H](N)Cc2c1cc(O)c(N1CC(=O)NS1(=O)=O)c2F] |
| 399 | 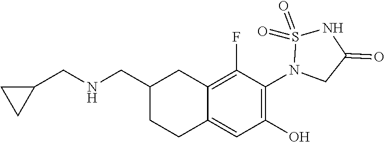 [SMILES: O=C1CN(c2c(O)cc3c(c2F)CC(CNCC2CC2)CC3)S(=O)(=O)N1] |
| 400 | 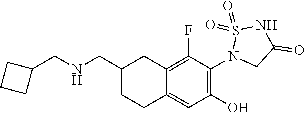 [SMILES: O=C1CN(c2c(O)cc3c(c2F)CC(CNCC2CCC2)CC3)S(=O)(=O)N1] |
| 401 | 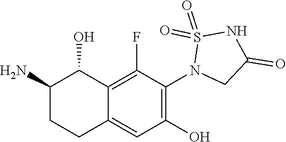 [SMILES: N[C@@H]1CCc2cc(O)c(N3CC(=O)NS3(=O)=O)c(F)c2[C@H]1O] |
| 402 | 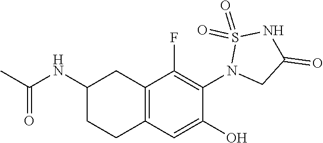 [SMILES: CC(=O)NC1CCc2cc(O)c(N3CC(=O)NS3(=O)=O)c(F)c2C1] |
| 403 | 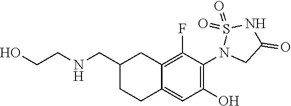 [SMILES: O=C1CN(c2c(O)cc3c(c2F)CC(CNCCO)CC3)S(=O)(=O)N1] |
| 404 | 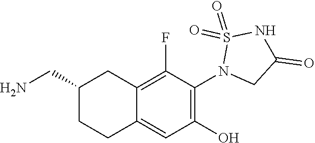 [SMILES: NC[C@H]1CCc2cc(O)c(N3CC(=O)NS3(=O)=O)c(F)c2C1] |
| 405 | 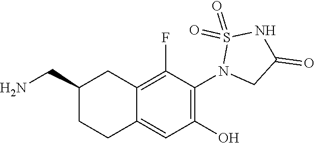 [SMILES: NC[C@@H]1CCc2cc(O)c(N3CC(=O)NS3(=O)=O)c(F)c2C1] |
| 406 | 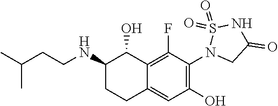 [SMILES: CC(C)CCN[C@@H]1CCc2cc(O)c(N3CC(=O)NS3(=O)=O)c(F)c2[C@H]1O] |
| 407 | 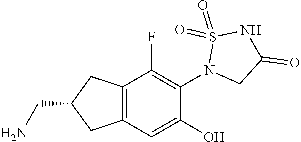 [SMILES: NC[C@H]1Cc2cc(O)c(N3CC(=O)NS3(=O)=O)c(F)c2C1] |
| 408 | 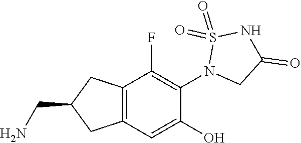 [SMILES: NC[C@@H]1Cc2cc(O)c(N3CC(=O)NS3(=O)=O)c(F)c2C1] |
| 409 | 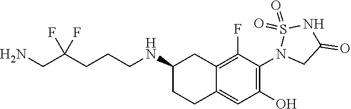 [SMILES: NCC(F)(F)CCCN[C@@H]1CCc2cc(O)c(N3CC(=O)NS3(=O)=O)c(F)c2C1] |
| 410 | 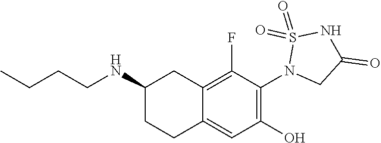 [SMILES: CCCCN[C@@H]1CCc2cc(O)c(N3CC(=O)NS3(=O)=O)c(F)c2C1] |
| 411 | 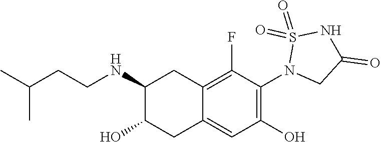 [SMILES: CC(C)CCN[C@H]1Cc2c(cc(O)c(N3CC(=O)NS3(=O)=O)c2F)C[C@@H]1O] |

### Methods of Making Exemplary Compounds

The compounds of the present disclosure may be better understood in connection with the following synthetic schemes and methods which illustrate a means by which the compounds can be prepared. The compounds of the present disclosure can be prepared by a variety of 5 synthetic procedures. Representative synthetic procedures are shown in, but not limited to, Schemes 1-24. The variables R¹, R^{II1}, R^{III1′}, R^{III1}, R², R³, R^{II3}, R^{II3′}, R^{III3}, R⁴, R⁵, R^{II5}, R^{III5}, R⁶, R^{II6}, R^{III6}, R⁷, R^{II7}, R^{III7}, R^{a}, and R^{c} are defined as detailed herein, e.g., in the Summary.

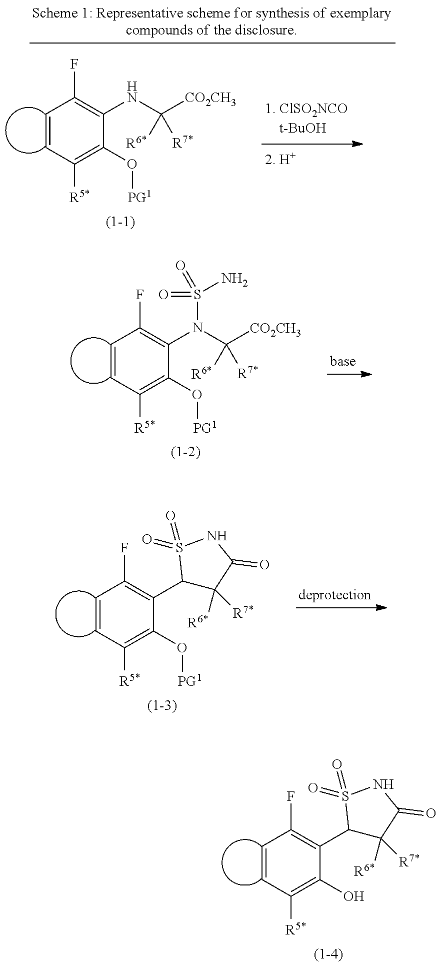

As shown in Scheme 1, compounds of formula (1-4) can be prepared from compounds of formula (1-1), wherein R^{5*} is R⁵, R^{II5}, or R^{III5}, R^{6*} is R⁶, R^{II6}, or R^{III6}, and R^{7*} is R⁷, R^{II7}, or R^{III7}. Compounds of formula (1-1), prepared as described in the Examples and Schemes below, wherein PG¹ is a protecting group such as benzyl, can be reacted in a first step with a preformed mixture of chlorosulfonyl isocyanate and tert-butanol in a solvent such as but not limited to cooled (−10-10° C.) dichloromethane in the presence of a tertiary amine base such as triethylamine or diisopropylethylamine. The intermediate can then be treated under acidic conditions such as trifluoroacetic acid in dichloromethane or hydrochloric acid in dioxane to give compounds of formula (1-2). Compounds of formula (1-2) can be reacted with an alkoxide base such as sodium methoxide in a solvent such as tetrahydrofuran at or near ambient temperature to give compounds of formula (1-3). The protecting group, PG¹, of compounds of formula (1-3) can be removed to give compounds of formula (1-4). When PG¹ is a benzyl group, the deprotection can be accomplished by catalytic hydrogenation. Compounds of formula (1-4) are representative of compounds of formula (I), formula (II), and formula (III).

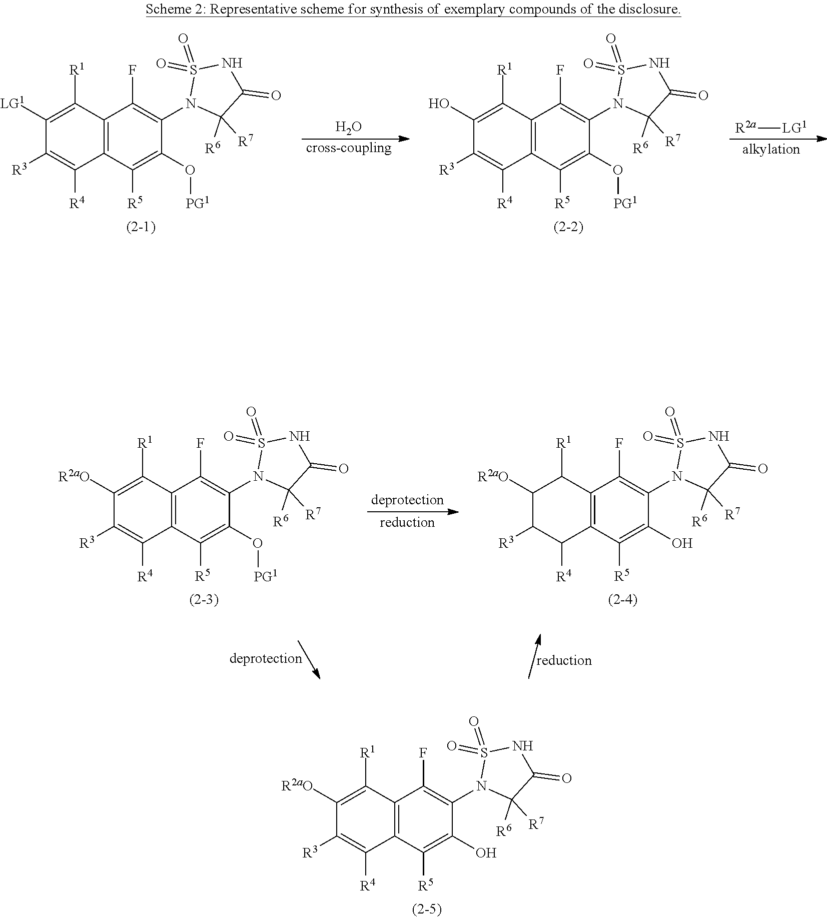

As shown in Scheme 2, compounds of formula (2-4) can be prepared from compounds of formula (2-1), wherein LG¹ is a leaving group such as chlorine, bromine, iodine or a sulfonate and PG¹ is a protecting group such as but not limited to benzyl. Compounds of formula (2-1) can be cross-coupled with water under palladium-catalyzed cross-coupling conditions including a catalyst or precatalyst, a base such as cesium carbonate, and a heated solvent mixture such as N,N-dimethylformamide and water to give compounds of formula (2-2). Compounds of formula (2-2) can be alkylated with compounds of formula R^{2a}-LG¹, wherein R^{2a} is an optionally substituted C₁₋₆alkyl, C₃₋₆cycloalkylC₁₋₆alkylene, or (3-6-membered heterocyclyl) C₁₋₆alkylene and LG¹ is a leaving group such as chlorine, bromine, iodine or a sulfonate in the presence of a base such as cesium carbonate and a solvent such as N,N-dimethylformamide to give compounds of formula (2-3). Compounds of formula (2-3) can be transformed to compounds of formula (2-4) using catalytic hydrogenation (130-150 psi) over 30-50 hours in a solvent such as 2,2,2-trifluoroethanol to remove both the protecting group, PG¹, and reduce the aromatic ring.

Alternatively, compounds of formula (2-3) can be deprotected using methodologies known to one of skill in the art to give compounds of formula (2-5). When PG¹ is benzyl, treatment of compounds of formula (2-3) with boron trichloride in the presence of pentamethylbenzene in cold dichloromethane gives compounds of formula (2-5). Compounds of formula (2-5) can then be converted under catalytic hydrogenation conditions in acetic acid to compounds of formula (2-4). Compounds of formula (2-4) are representative of compounds of formula (I).

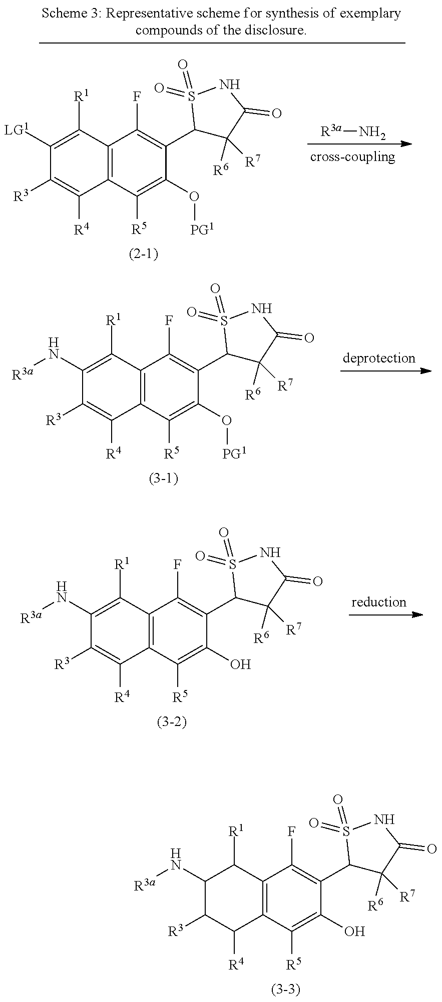

As shown in Scheme 3, compounds of formula (3-3) can be prepared from compounds of formula (2-1), wherein LG¹ is a leaving group such as chlorine, bromine, iodine or a sulfonate and PG¹ is a protecting group such as but not limited to benzyl. Compounds of formula (2-1) can be cross-coupled with amines of formula R^{3a}—NH₂, wherein R^{3a} is an optionally substituted C₁₋₆alkyl, optionally substituted C₁₋₆cycloalkylC₁₋₆alkylene, optionally substituted 4-6-membered heterocyclyl, optionally substituted (4-6-membered heterocyclyl) C₁₋₆alkylene, optionally substituted (5-6-membered heteroaryl) C₁₋₆alkylene or optionally substituted phenyl-C₁₋₆alkylene under palladium-catalyzed cross-coupling conditions including a catalyst or precatalyst, a ligand a base such as cesium carbonate, and a heated solvent such as tert-amyl alcohol to give compounds of formula (3-1). Compounds of formula (3-1) can be deprotected as described in Scheme 2 to give compounds of formula (3-2). Compounds of formula (3-2) can be reduced to compounds of formula (3-3) using catalytic hydrogenation conditions in acetic acid or a mixture of methanol and acetic acid. Compounds of formula (3-3) are representative of compounds of formula (I).

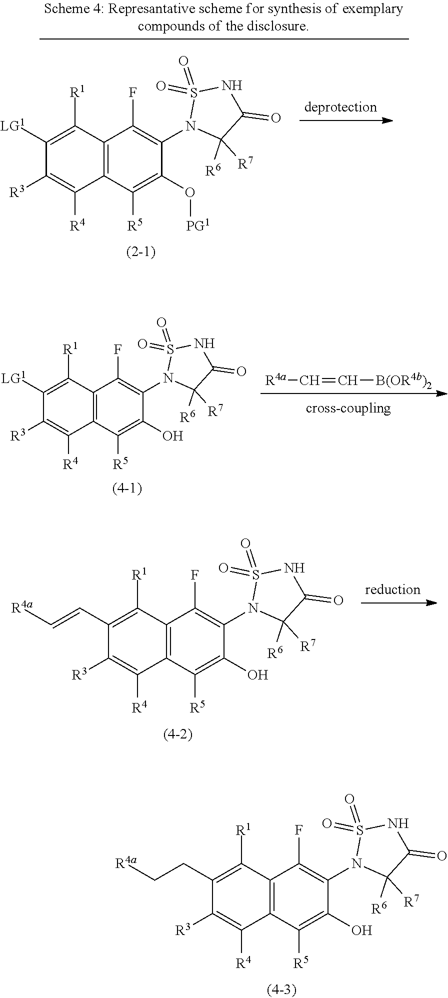

As shown in Scheme 4, compounds of formula (4-3) can be prepared from compounds of formula (2-1), wherein LG¹ is a leaving group such as chlorine, bromine, iodine or a sulfonate and PG¹ is a protecting group such as but not limited to benzyl. Compounds of formula (2-1) can be deprotected using methodologies known to one of skill in the art to give compounds of formula (4-1). When PG¹ is benzyl, treatment of compounds of formula (2-1) with boron trichloride in the presence of pentamethylbenzene in cold dichloromethane gives compounds of formula (4-1). Compounds of formula (4-1) can be cross-coupled with R^{4a}—CH═CH—B(OR^{4b})₂, wherein —B(OR^{4b})₂ represents a boronic acid or boronate and R^{4a} is an optionally substituted C₃₋₆cycloalkyl and optionally substituted 4-6-membered heterocyclyl, for example under Suzuki reaction conditions to give compounds of formula (4-2). Reduction of compounds of formula (4-2) under catalytic hydrogenation conditions in a solvent such as but not limited to 2,2,2-trifluoroethanol gives compounds of formula (4-3). Compounds of formula (4-3) are representative of compounds of formula (I).

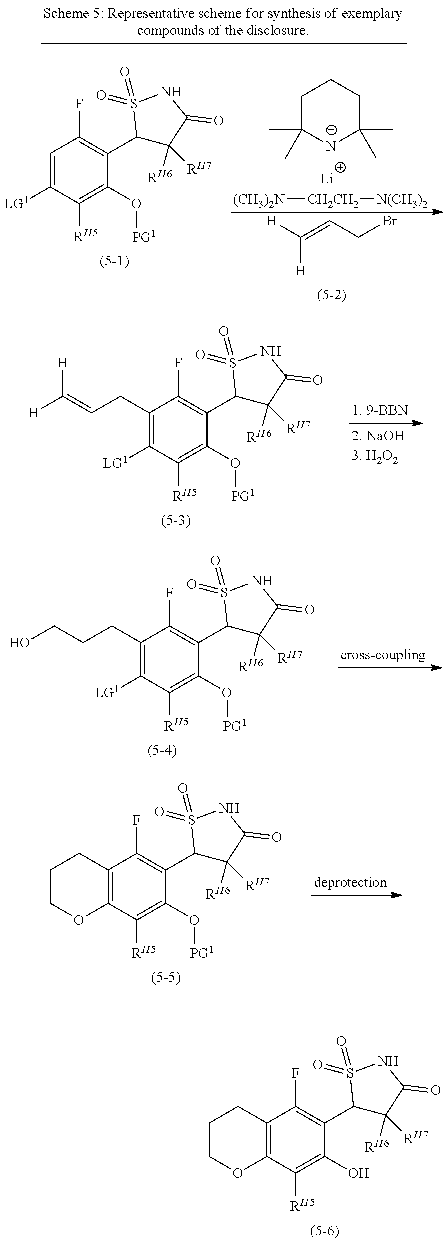

As shown in Scheme 5, compounds of formula (5-6) can be prepared from compounds of formula (5-1), wherein LG¹ is a leaving group such as chlorine, bromine, iodine or a sulfonate and PG¹ is a protecting group such as but not limited to benzyl. Compounds of formula (5-1) can be reacted with a base such as lithium 2,2,6,6-tetramethylpiperidine-1-ide in the presence of N,N,N′,N′-tetramethylethylenediamine in a solvent such as cold tetrahydrofuran and then treated with allyl bromide, (5-2), to give compounds of formula (5-3). Compounds of formula (5-3) can be treated in a three-step hydroboration-oxidation sequence to give compounds of formula (5-4). Then, compounds of formula (5-4) can be reacted under appropriate palladium-catalyzed cross-coupling reaction conditions to give chromanes of formula (5-5). Compounds of formula (5-5) can be deprotected using methodologies known to one of skill in the art to give compounds of formula (5-6). When PG¹ is benzyl, treatment of compounds of formula (5-5) with boron trichloride in the presence of pentamethylbenzene in cold dichloromethane gives compounds of formula (5-6). Compounds of formula (5-6) are representative of compounds of formula (II).

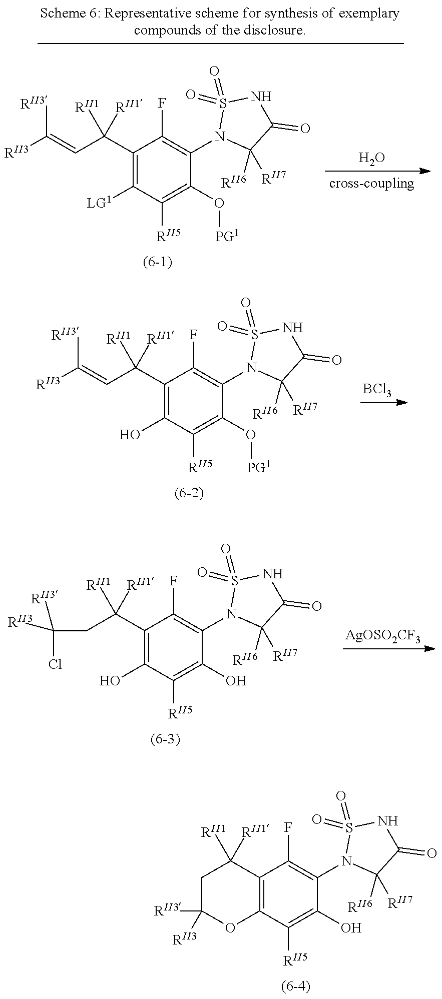

As shown in Scheme 6, compounds of formula (6-4) can be prepared from compounds of formula (6-1), wherein LG¹ is a leaving group such as chlorine, bromine, iodine or a sulfonate and PG¹ is a protecting group such as but not limited to benzyl. Compounds of formula (6-1), prepared analogously to compounds of formula (5-3) in Scheme 5, can be cross-coupled with water to give compounds of formula (6-2). Compounds of formula (6-2) can be reacted with boron trichloride in the presence of pentamethylbenzene in cold dichloromethane gives compounds of formula (6-3). Compounds of formula (6-3) can be cyclized in the presence of a silver salt such as silver trifluoromethanesulfonate to give compounds of formula (6-4). Compounds of formula (6-4) are representative of compounds of formula (II).

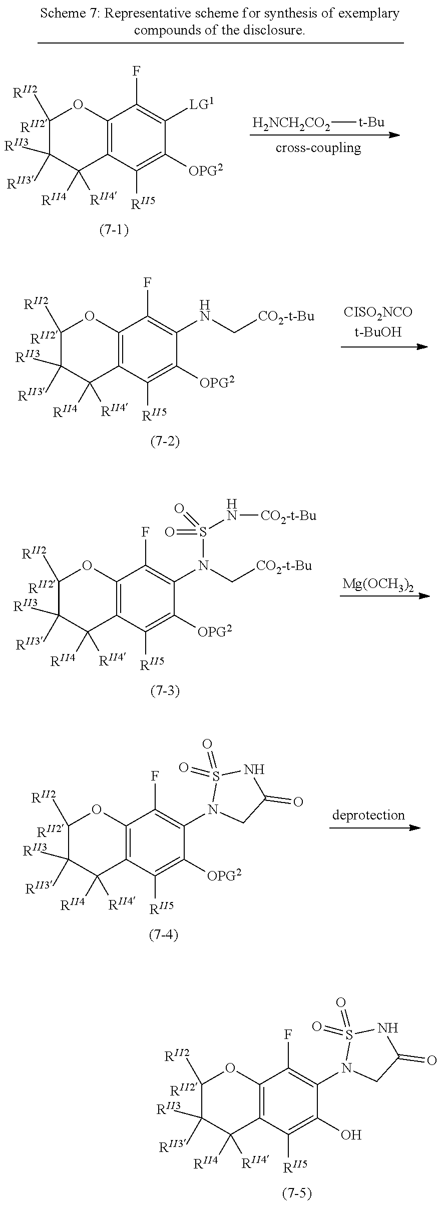

As shown in Scheme 7, compounds of formula (7-5) can be prepared from compounds of formula (7-1), wherein LG¹ is a leaving group such as chlorine, bromine, iodine or a sulfonate and PG² is a protecting group such as but not limited to (methoxyethoxy)methyl. Compounds of formula (7-1) can be cross-coupled with H₂NCH₂CO₂-t-Bu in the presence of a palladium catalyst, ligand, and base to give compounds of formula (7-2). Compounds of formula (7-2) can then be reacted with a preformed mixture of chlorosulfonyl isocyanate and tert-butanol in a solvent such as but not limited to cooled dichloromethane in the presence of a tertiary amine base such as triethylamine or diisopropylethylamine to give compounds of formula (7-3). Compounds of formula (7-3) can then be reacted with Mg(OCH₃)₂ in methanol in a heated solvent such as 2-methyltetrahydrofuran to give the cyclized compounds of formula (7-4). Compounds of formula (7-4) can then be converted to compounds of formula (7-5) by removal of the protecting group, PG², under conditions known to one of skill in the art and dependent upon the particular protecting group. When PG² is (methoxyethoxy)methyl, treatment with an acid such as 4 M HCl in dioxane gives compounds of formula (7-5). Compounds of formula (7-5) are representative of compounds of formula (II).

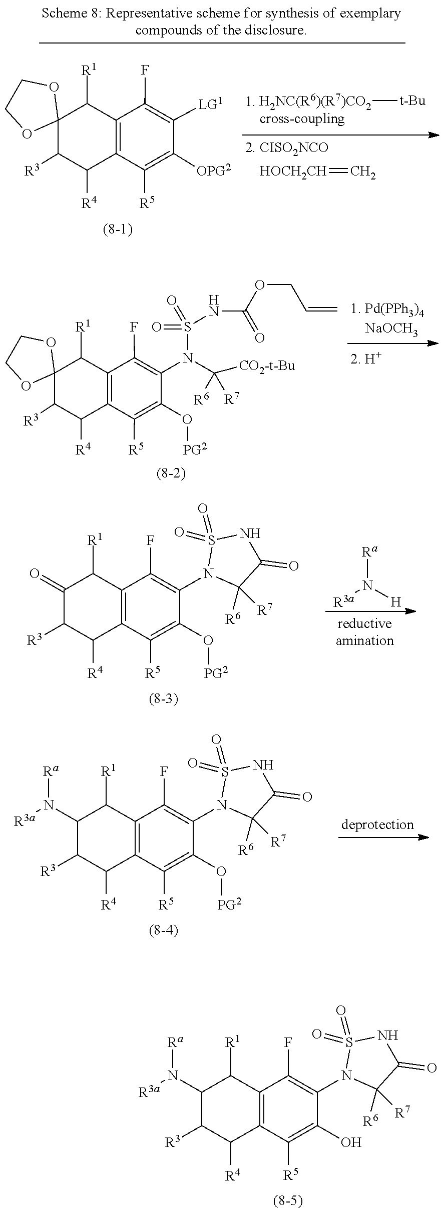

As shown in Scheme 8, compounds of formula (8-5) can be prepared from compounds of formula (8-1), wherein LG¹ is a leaving group such as chlorine, bromine, iodine or a sulfonate and PG² is a protecting group such as but not limited to (methoxyethoxy)methyl. Accordingly, compounds of formula (8-1) can be cross-coupled with amines, H₂NC(R⁶)(R⁷)CO₂-t-Bu, under conditions known to one of skill in the art. Subsequent treatment with a preformed mixture of chlorosulfonyl isocyanate and allyl alcohol in a solvent such as chilled dichloromethane gives compounds of formula (8-2). Treatment of compounds of formula (8-2) with tetrakis(triphenylphosphine)palladium(0) in the presence of a base such as sodium methoxide can give the corresponding 1λ⁶,2,5-thiadiazolidine-1,1,3-trione moiety. Then the dioxolane moiety can be removed by treatment under acidic conditions such as but not limited to formic acid to give compounds of formula (8-3). Compounds of formula (8-3) can be reductively aminated with amines, (R^{3a})(R^{a}) NH wherein R^{3a} is as described in Scheme 3, to give compounds of formula (8-4). Alternatively, R^{3a} and R^{a} and the nitrogen to which they are attached may be joined to form a 4-8 membered heterocycle which can be used to reductively aminate the compounds of formula (8-3). The protecting group, PG², can be removed known to one of skill in the art and dependent upon the particular protecting group to give compounds of formula (8-5). When PG² is (methoxyethoxy)methyl, treatment with an acid such as 4 M HCl in dioxane gives compounds of formula (8-5). Compounds of formula (8-5) are representative of compounds of formula (I).

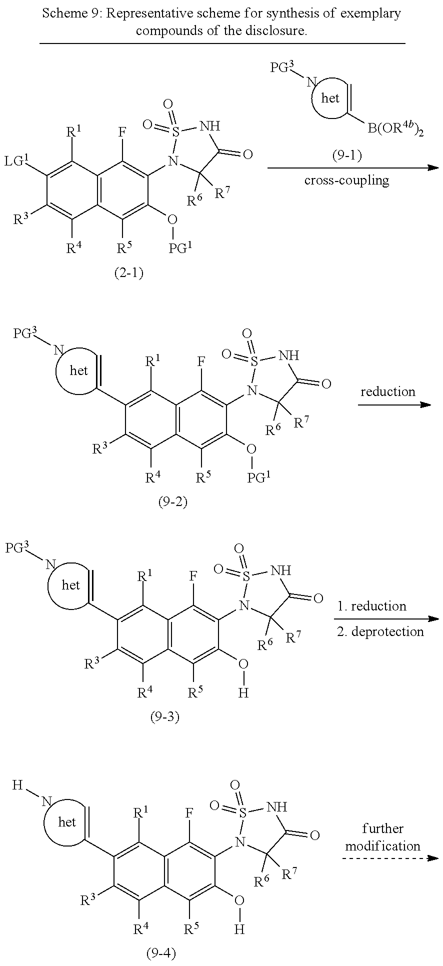

As shown in Scheme 9, compounds of formula (9-4) can be prepared from compounds of formula (2-1), wherein LG¹ is a leaving group such as chlorine, bromine, iodine or a sulfonate and PG¹ is a protecting group such as but not limited to benzyl. Compounds of formula (2-1) can be cross-coupled under palladium-catalyzed reaction conditions such as Suzuki reaction conditions with compounds of formula (9-1), wherein —B(OR^{4b})₂ represents a boronic acid or boronate, PG³ is an amine protecting group such as tert-butoxycarbonyl, and “het” is a heterocyclyl containing a ring nitrogen, to give compounds of formula (9-2). Treatment under catalytic hydrogenation conditions saturates the heterocyclyl ring and removes the protecting group, PG¹, to give compounds of formula (9-3). Compounds of formula (9-3) can be reduced further with catalytic hydrogenation conditions, and the protecting group, PG³, can be removed in a second step to give compounds of formula (9-4). When PG³ is tert-butoxycarbonyl, treatment with an acid such as trifluoroacetic acid in dichloromethane is suitable for protecting group removal. Compounds of formula (9-4) are representative of compounds of formula (I). Compounds of formula (9-4) can be further modified such as by alkylation or acylation to give additional compounds of formula (I).

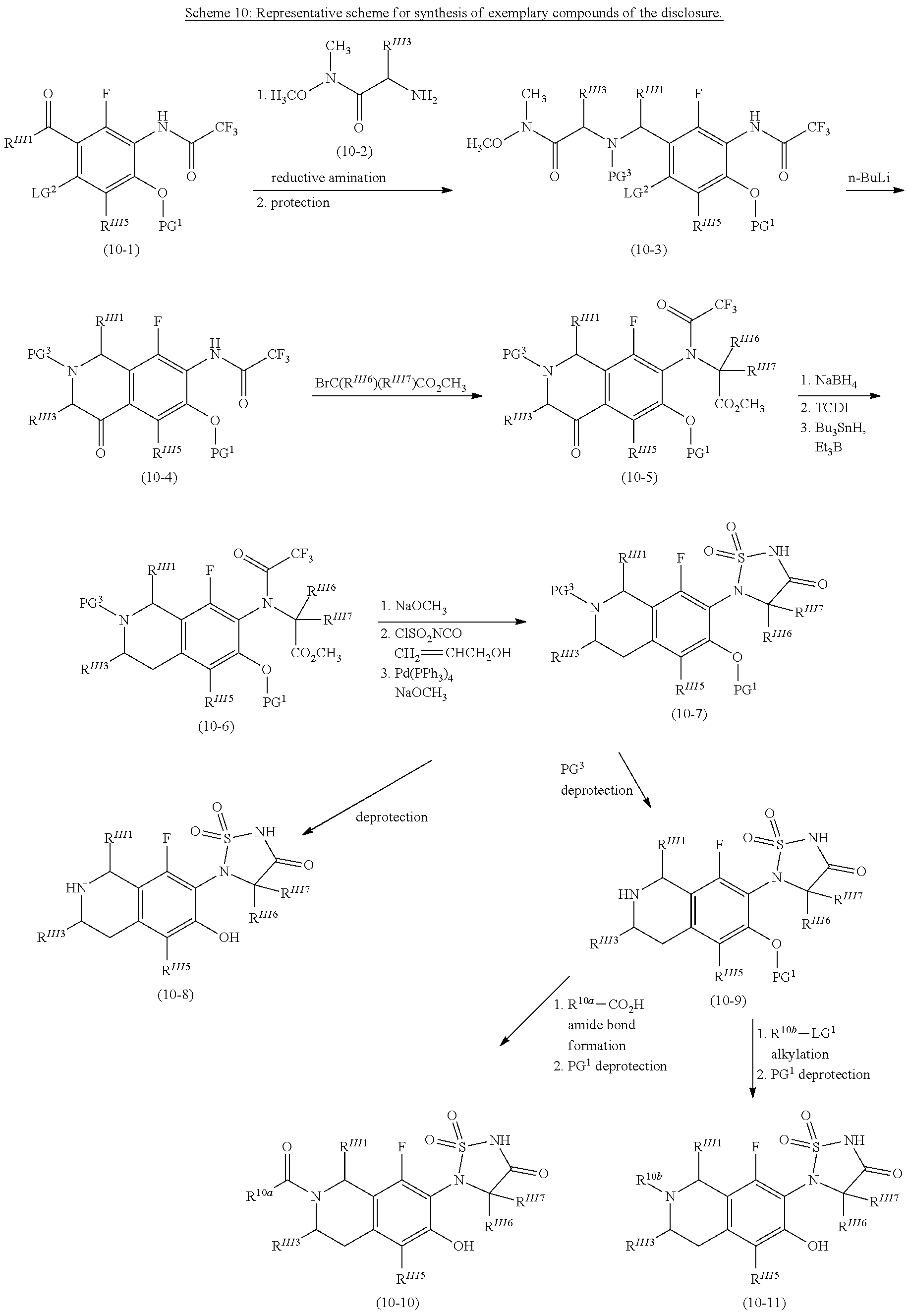

As shown in Scheme 10, compounds of formula (10-8), formula (10-10), and formula (10-11) can be prepared from compounds of formula (10-1), wherein LG² is a leaving group such as chlorine, bromine, or iodine and PG¹ is a protecting group such as but not limited to benzyl. Compounds of formula (10-1) can be reacted with compounds of formula (10-2) under reductive amination conditions. Subsequent protection of the formed amine with a nitrogen protecting group such as but not limited to tert-butoxycarbonyl provides compounds of formula (10-3). Compounds of formula (10-3) can be treated with n-butyllithium resulting in cyclization to give compounds of formula (10-4). The amide nitrogen of compounds of formula (10-4) can be alkylated with a bromoacetate. BrC(R^{III6})(R^{III7})CO₂CH₃, in the presence of a base such as but not limited to 1,2,2,6,6-pentamethylpiperidine in a heated solvent such as heated N,N-dimethylformamide to give compounds of formula (10-5). In a three-step process, the carbonyl can be reduced to the corresponding methylene. In the first step, treatment with a reductant such as sodium borohydride provides the corresponding alcohols. The second step is treatment with 1,1′-thiocarbonyldiimidazole (TCDI) in the presence of a base to give the corresponding 1H-imidazole-1-carbothioates. The 1H-imidazole-1-carbothioates can be treated tributyltin hydride and triethylborane in the third step with to give the tetrahydroisoquinolines of formula (10-6). Compounds of formula (10-6) can be treated with sodium methoxide in warmed methanol to remove the trifluoroacetyl moiety. Subsequent treatment with a preformed mixture of chlorosulfonyl isocyanate and allyl alcohol in a solvent such as chilled dichloromethane gives alloc-sulfonylureas. Treatment of the alloc-sulfonylureas with tetrakis(triphenylphosphine)palladium(0) in the presence of a base such as sodium methoxide can give the corresponding 1λ⁶,2,5-thiadiazolidine-1,1,3-trione moiety of compounds of formula (10-7). Simultaneous removal of protecting groups PG¹ and PGR give compounds of formula (10-8). When PG¹ is benzyl and PG³ is tert-butoxycarbonyl, treatment with boron trichloride in the presence of 1,2,3,4,5-pentamethylbenzene in cold dichloromethane removes both the benzyl and tert-butoxy carbonyl groups. Alternatively, the protecting group, PG³, can be selectively removed from compounds of formula (10-7) to give compounds of formula (10-9). When PG³ is tert-butoxycarbonyl, treatment with an acid such as trifluoroacetic acid in dichloromethane gives compounds of formula (10-9). Compounds of formula (10)-9) can be treated with carboxylic acids of formula R^{10a}—CO₂H, wherein R^{10a} is optionally substituted C₁₋₆alkyl, under amide bond forming conditions, and then have PG¹ subsequently removed to give compounds of formula (10-10). One set of amide bond forming conditions involves treatment with 2-(1H-benzotriazole-1-yl)-1,1,3,3-tetramethylaminium tetrafluoroborate (TBTU) in the presence of a tertiary amine base such as diisopropylethylamine in a solvent such as dichloromethane. When PG¹ is benzyl, treatment with boron trichloride in the presence of 1,2,3,4,5-pentamethylbenzene in cold dichloromethane removes the benzyl protecting group. Compounds of formula (10-9) can also be alkylated with compounds of formula R^{10b}-LG¹, wherein R^{10b} is optionally substituted C₁₋₆alkyl, optionally substituted-C₁₋₆alkylene-C₃₋₆cycloalkyl, optionally substituted C₁₋₆alkylene-phenyl, optionally substituted C₁₋₆alkylene-(4-6-membered) heterocyclyl and optionally substituted C₁₋₆alkylene-(5-6-membered) heteroaryl and LG¹ is a leaving group such as chlorine, bromine, iodine or a sulfonate, and then deprotected to give compounds of formula (10-11). One set of alkylation conditions involves treatment of compounds of formula (10-9) with compounds of formula R^{10b}-LG¹ in the presence of a base such as cesium carbonate in warmed acetonitrile. Then when PG¹ is benzyl, treatment with boron trichloride in the presence of 1,2,3,4,5-pentamethylbenzene in cold dichloromethane removes the benzyl protecting group and provides compounds of formula (10-11). Compounds of formula (10-8), formula (10-10), and formula (10-11) are representative of compounds of formula (III).

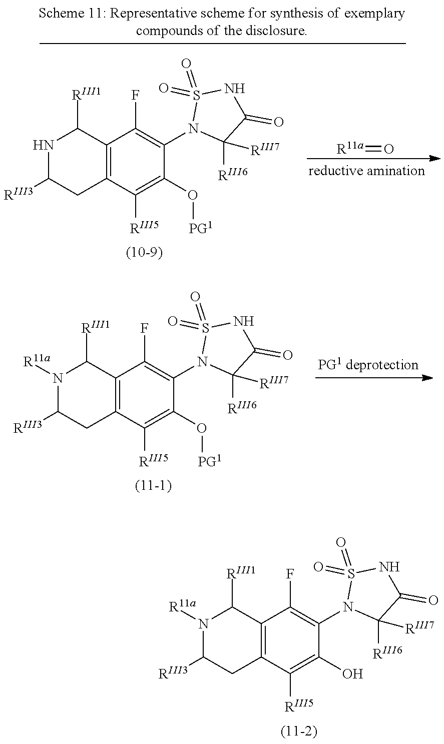

As shown in Scheme 11, compounds of formula (11-2) can be prepared from compounds of formula (10-9), wherein PG¹ is a protecting group such as but not limited to benzyl. Compounds of formula (10-9) can be reacted with compounds of formula R^{11a}═O under reductive amination conditions to give compounds of formula (11-1). R^{11a} is optionally substituted C₁₋₆alkyl, C₂₋₆alkenyl, C₂₋₆alkynyl, C₃₋₆cycloalkyl, —C₁₋₆alkylene-C₃₋₇cycloalkyl, —C₁₋₆alkylene-phenyl, —C₁₋₆alkylene-4-6 membered heterocyclyl, —C₁₋₆alkylene-5-6 membered heteroaryl, 4-8 membered heterocycle, —(4-7 membered-heterocycle)-C₁₋₆alkylene-5-6 membered heteroaryl. R^{11a} may be optionally substituted as described for R^{III2}. R^{11a}═O is the corresponding aldehyde or corresponding ketone of C₁₋₆alkyl, C₂₋₆alkenyl, C₂₋₆alkynyl, C₃₋₆cycloalkyl, H—C₁₋₆alkylene-C₃₋₇cycloalkyl, H—C₁₋₆alkylene-phenyl, H—C₁₋₆alkylene-4-6 membered heterocyclyl, H—C₁₋₆alkylene-5-6 membered heteroaryl, 4-8 membered heterocycle, H-(4-7 membered-heterocycle)-C₁₋₆alkylene-5-6 membered heteroaryl. Compounds of formula (11-1) can be deprotected using methods known to one of skill in the art and dependent upon the nature of PG¹ to give compounds of formula (11-1). When PG¹ is benzyl, treatment with boron trichloride in the presence of 1,2,3,4,5-pentamethylbenzene in cold dichloromethane removes the benzyl protecting group and provides compounds of formula (11-2). Alternatively, when PG¹ is benzyl, treatment under catalytic or transfer hydrogenation conditions removes the benzyl protecting group providing compounds of formula (11-2). Compounds of formula (11-1) and compounds of formula (11-2) can be further modified using methodologies known to one of skill in the art. Compounds of formula (11-2) are representative of compounds of formula (III).

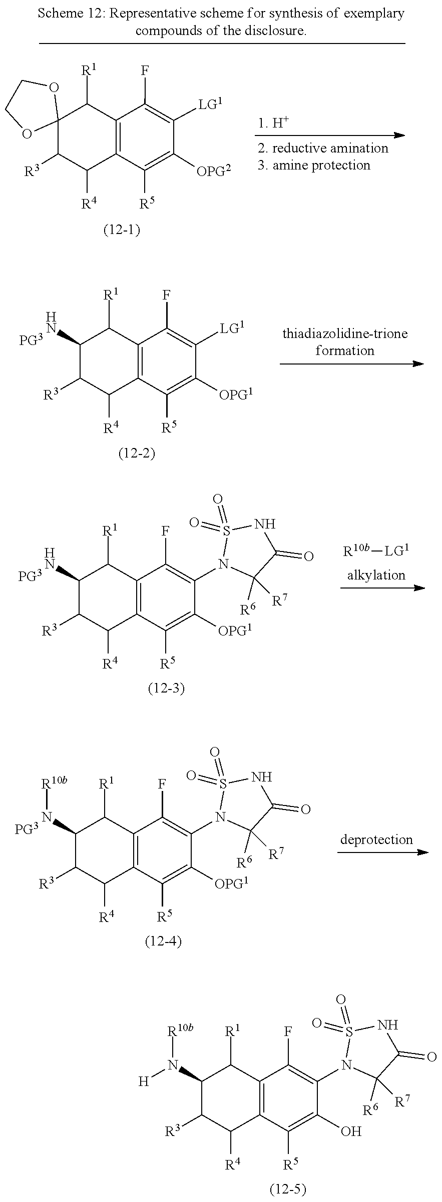

As shown in Scheme 12, compounds of formula (12-5) can be prepared from compounds of formula (12-1), wherein R^{10b} is optionally substituted C₁₋₆alkyl, optionally substituted-C₁₋₆alkylene-C₃₋₆cycloalkyl, optionally substituted C₁₋₆alkylene-phenyl, optionally substituted C₁₋₆alkylene-(4-6-membered) heterocyclyl and optionally substituted C₁₋₆alkylene-(5-6-membered) heteroaryl. Compounds of formula (12-1), wherein PG¹ is a protecting group such as benzyl, can be converted to compound of formula (12-2) in a three-step process. The dioxolane of compounds of formula (12-1) can be removed under acidic conditions known to one of skill in the art. A reductive amination can introduce an amine moiety, —NH₂. The reductive amination can be done under conditions known to one of skill in the art. One stereoselective set of conditions include treatment with monobasic sodium phosphate, hydrochloric acid, sec-butylamine, pyridoxal-5-phosphate, and Codexis® ATA-025. The resulting amine can be protected as a benzyloxycarbonyl by treatment with benzyl chloroformate in the presence of a base forming a benzyloxycarbonyl, PG³, protecting group. Compounds of formula (12-2) can then be transformed to compounds of formula (12-3) using the thiadiazolidine-trione forming sequence described in Scheme 8. Compounds of formula (12-2) can be alkylated with R^{10b}-LG¹ as described in Scheme 10 to give compounds of formula (12-4). Dependent on PG¹ and PG³, the protecting groups of compounds of formula (12-4) can be removed stepwise or simultaneously to give compounds of formula (12-5). For example, when PG¹ is benzyl and PG³ is benzyloxycarbonyl, treatment with boron trichloride in the presence of pentamethylbenzene in cold dichloromethane simultaneously removes both protecting groups. Compounds of formula (12-5) are representative of compounds of formula (I).

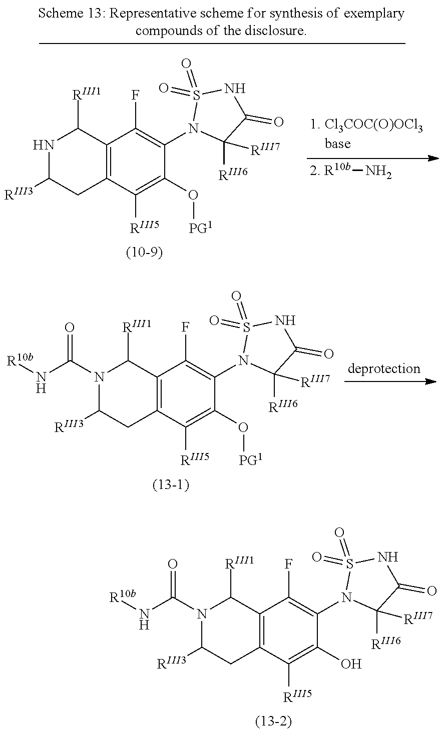

As shown in Scheme 13, compounds of formula (13-2) can be prepared from compounds of formula (10-9). Compounds of formula (10-9) can be reacted with triphosgene in the presence of a base such as but not limited to a tertiary amine. Subsequent treatment with an amine, R^{10b}—NH₂, wherein R^{10b} is optionally substituted C₁₋₆alkyl, optionally substituted —C₁₋₆alkylene-C₃₋₆cycloalkyl, optionally substituted C₁₋₆alkylene-phenyl, optionally substituted C₁₋₆alkylene-(4-6-membered) heterocyclyl and optionally substituted C₁₋₆alkylene-(5-6-membered) heteroaryl, provides compounds of formula (13-1). Removal of the protecting group, PG¹, is accomplished using methodologies known to one of skill in the art and dependent upon PG¹. When PG¹ is benzyl, treatment with boron trichloride in the presence of 1,2,3,4,5-pentamethylbenzene in cold dichloromethane removes the benzyl protecting group and provides compounds of formula (13-2). Compounds of formula (13-2) are representative of compounds of formula (III).

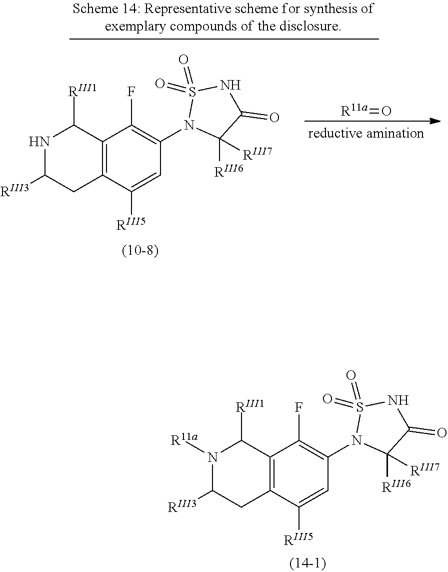

As shown in Scheme 14, compounds of formula (14-1) can be prepared from compounds of formula (10-8). Compounds of formula (10-8) can be reacted with compounds of formula R^{11a}═O under reductive amination conditions to give compounds of formula (14-1). R^{11a} is optionally substituted C₁₋₆alkyl, C₂₋₆alkenyl, C₂₋₆alkynyl, C₃₋₆cycloalkyl, —C₁₋₆alkylene-C₃₋₇cycloalkyl, —C₁₋₆alkylene-phenyl, —C₁₋₆alkylene-4-6 membered heterocyclyl, —C₁₋₆alkylene-5-6 membered heteroaryl, 4-8 membered heterocycle, —(4-7 membered-heterocycle)-C₁₋₆alkylene-5-6 membered heteroaryl. R^{11a} may be optionally substituted as described for R^{III2}. R^{11a}═O is the corresponding aldehyde or corresponding ketone of C₁₋₆alkyl, C₂₋₆alkenyl, C₂₋₆alkynyl, C₃₋₆cycloalkyl, H—C₁₋₆alkylene-C₃₋₇cycloalkyl, H—C₁₋₆alkylene-phenyl, H—C₁₋₆alkylene-4-6 membered heterocyclyl, H—C₁₋₆alkylene-5-6 membered heteroaryl, 4-8 membered heterocycle, H-(4-7 membered-heterocycle)-C₁₋₆alkylene-5-6 membered heteroaryl. The reductive amination can be conducted with conventional reagents such as sodium cynanoborohydride or sodium borohydride or the solid supported equivalents. Compounds of formula (14-1) can be further modified using methodologies known to one of skill in the art. Compounds of formula (14-1) are representative of compounds of formula (III).

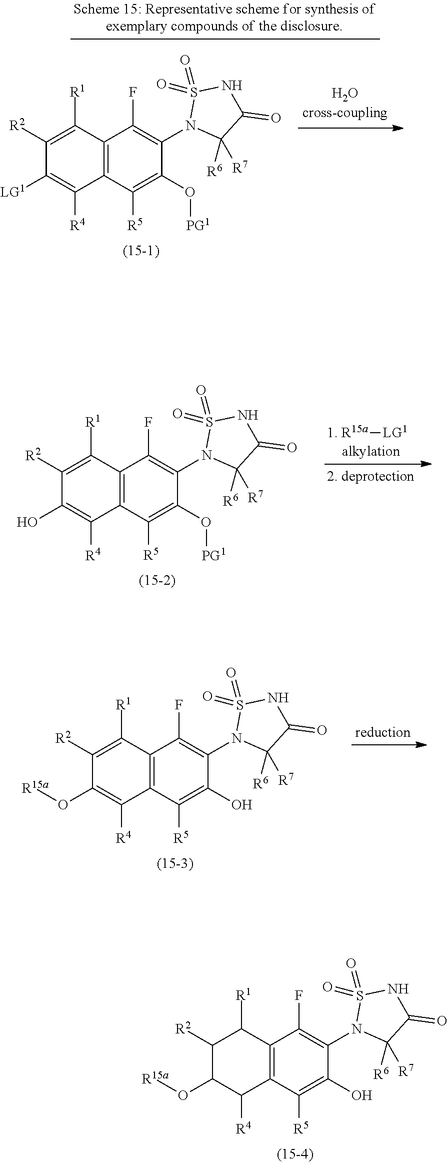

As shown in Scheme 15, compounds of formula (15-4) can be prepared from compounds of formula (15-1). Compounds of formula (15-1) can cross-coupled with water under palladium-catalyzed cross-coupling conditions including a catalyst or precatalyst, an optional ligand, a base such as cesium carbonate, and a heated solvent such as N,N-dimethylacetamide to give compounds of formula (15-2). Compounds of formula (15-2) can be alkylated with compounds of formula R^{15a}-LG¹, wherein R^{15a} is optionally substituted C₁₋₆alkyl, optionally substituted-C₁₋₆alkylene-C₃₋₆cycloalkyl, optionally substituted C₁₋₆alkylene-phenyl, optionally substituted C₁₋₆alkylene-(4-6-membered) heterocyclyl and optionally substituted C₁₋₆alkylene-(5-6-membered) heteroaryl and LG¹ is a leaving group such as chlorine, bromine, iodine or a sulfonate, and then deprotected to give compounds of formula (15-3). One set of alkylation conditions involves treatment of compounds of formula (15-2) with compounds of formula R^{15a}-LG¹ in the presence of a base such as cesium carbonate in N,N-dimethylformamide. Then when PG¹ is benzyl, treatment with ammonium formate in ethanol in the presence of 10% palladium on carbon removes the benzyl protecting group and provides compounds of formula (15-3). Compounds of formula (15-3) can be reduced with hydrogen (approximately 120 psi) in the presence of 10% palladium on carbon in a solvent such as trifluoroethanol to give compounds of formula (15-4). Compounds of formula (15-4) are representative of compounds of formula (I).

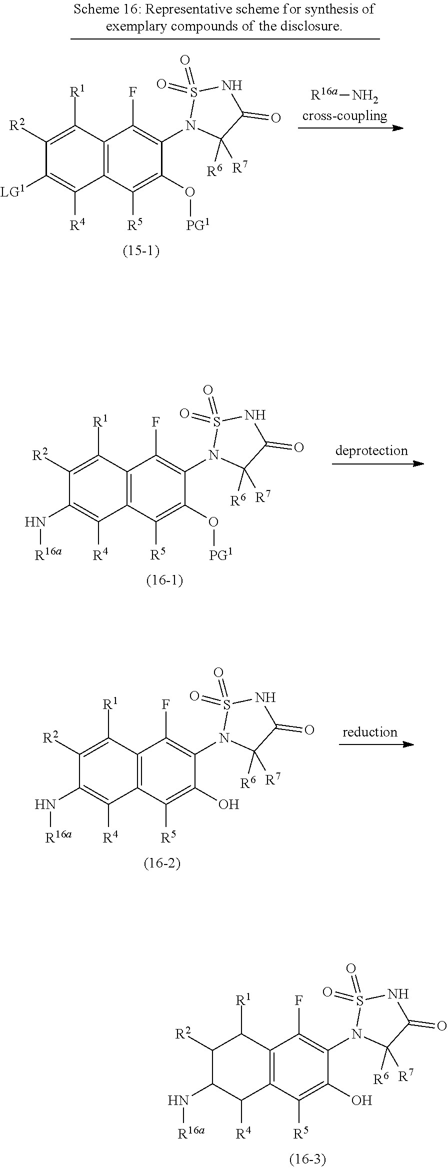

As shown in Scheme 16, compounds of formula (16-3) can be prepared from compounds of formula (15-1). Compounds of formula (15-1) can cross-coupled with amines, R^{16a}—NH₂, under palladium-catalyzed cross-coupling conditions including a catalyst or precatalyst, an optional ligand, a base such as cesium carbonate, and a heated solvent mixture such as N,N-dimethylacetamide to give compounds of formula (16-1). The protecting group, PG¹, can be removed under conditions known to one of skill in the art and dependent on the particular protecting group used. When PG¹ is benzyl, treatment with boron trichloride in the presence of pentamethylbenzene in cold dichloromethane or alternatively treatment under transfer hydrogenation conditions removes the protecting group giving compounds of formula (16-2). Compounds of formula (16-2) can be reduced with hydrogen (approximately 120 psi) in the presence of 10% palladium on carbon in a solvent such as trifluoroethanol to give compounds of formula (16-3). Compounds of formula (16-3) are representative of compounds of formula (I).

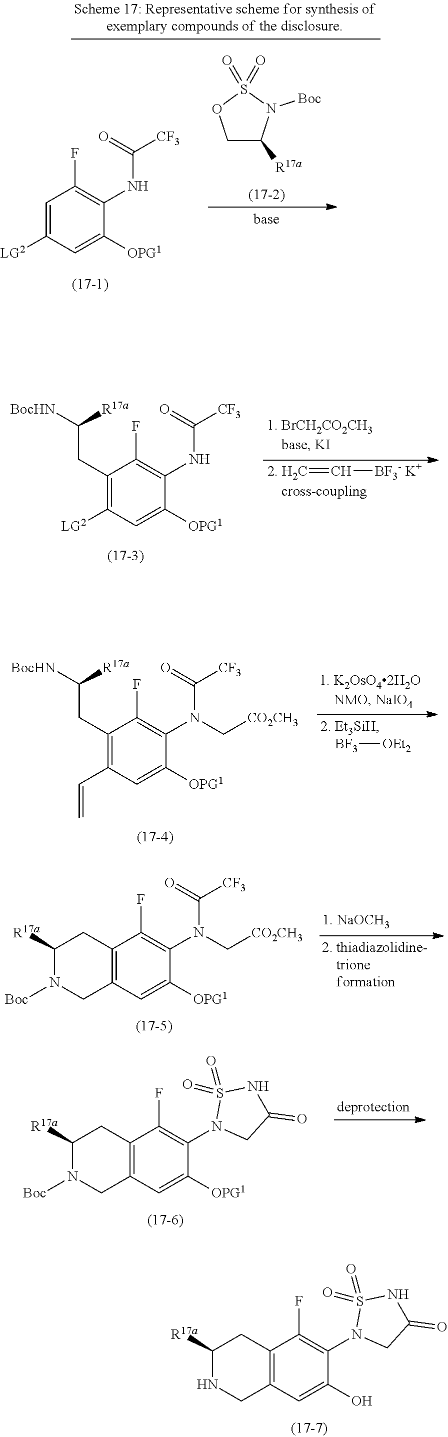

As shown in Scheme 17, compounds of formula (17-7) can be prepared from compounds of formula (17-1). Compounds of formula (17-1), wherein LG² is a leaving group such as chloro, bromo or iodo and PG¹ is a protecting group such as benzyl, can be treated with a base such lithium diisopropylamide and then with an oxathiazolidine 2,2-dioxide of formula (17-2), wherein Boc is tert-butoxycarbonyl and R^{17a} is optionally substituted alkyl, optionally substituted —C₁₋₆alkylene-C₃₋₆cycloalkyl, or optionally substituted-C₁₋₆alkylene-4-6 membered heterocyclyl, to give compounds of formula (17-3). Compounds of formula (17-3) can be alkylated with methyl bromoacetate in the presence of a base and potassium iodide. Then cross-coupling with potassium vinyltrifluoroborate supplies compounds of formula (17-4). Compounds of formula (17-4) can be oxidized to the corresponding aldehyde with potassium osmate dihydrate in the presence of N-methylmorpholine N-oxide and sodium metaperiodate. The intermediate aldehyde can be cyclized with triethylsilane in the presence boron trifluoride diethyl etherate to give compounds of formula (17-5). The trifluoroacetamide group can be removed from compounds of formula (17-5) by treatment with sodium methoxide. The thiadiazolidine-trione can be formed following the steps described in Scheme 8 giving compounds of formula (17-6). The protecting groups, Boc and PG¹, can be removed from compounds of formula (17-6) simultaneously or stepwise dependent on PG¹ using conditions known to one of skill in the art to give compounds of formula (17-7). For example, when PG¹ is benzyl, transfer hydrogenation will selectively remove PG¹. Subsequent exposure to hydrochloric acid in dioxane will remove the tert-butoxycarbonyl protecting group. Compounds of formula (17-7) are representative of compounds of formula (I).

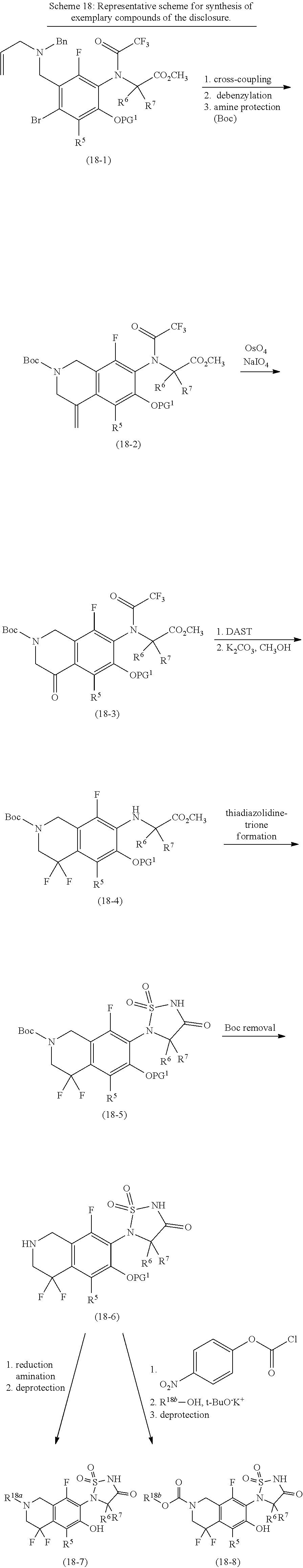

As shown in Scheme 18, compounds of formula (18-7) and compounds of formula (18-8) can be prepared from compounds of formula (18-1). Compounds of formula (18-1), wherein PG¹ is a protecting group such as benzyl, can be converted to compounds of formula (18-2) in a three-step process. Treating compounds of formula (18-1) under cross-coupling reaction conditions forms the bicyclic structure. The benzyl group can be selectively removed from the nitrogen of the tetrahydroisoquinoline by treatment with 1-chloroethyl chloroformate and 8-bis(dimethylamino)naphthalene in a solvent such as 1,2-dichloroethane. The exposed amine can be protected as the tert-butoxycarbonyl by treatment with di-tert-butyl dicarbonate in the presence of a base such as sodium bicarbonate in a solvent such as a mixture of tetrahydrofuran and water. Compounds of formula (18-2) can be oxidized with osmium tetroxide and sodium periodate to give the corresponding ketone, compounds of formula (18-3). Compounds of formula (18-3) can be treated with diethylaminosulfur trifluoride (DAST) to convert the ketone to the corresponding difluoromethylene. Subsequent treatment with potassium carbonate in methanol removes the trifluoroacetyl moiety to give compounds of formula (18-4). Compounds of formula (18-4) can be treated as described in Scheme 8 to construct the thiadiazolidine-trione giving compounds of formula (18-5). The tert-butoxycarbonyl protecting group can be removed from compounds of formula (18-5) by treatment under acidic conditions such as with trifluoroacetic acid in dichloromethane to give compounds of formula (18-6). Compounds of formula (18-6) can be reductively aminated and then deprotected using procedures known to one of skill in the art to give compounds of formula (18-7), wherein R^{18a} is C₁₋₆alkyl, C₂₋₆alkenyl, C₂₋₆alkynyl, C₃₋₆cycloalkyl, 4-7 membered heterocyclyl, —C₁₋₆alkylene-C₃₋₈cycloalkyl, —C₁₋₆alkylene-phenyl, —C₁₋₆alkylene-4-7 membered heterocyclyl, or —C₁₋₆alkylene-5-6 membered heteroaryl. Compounds of formula (18-6) can also be transformed to compounds of formula (18-8). Compounds of formula (18-6) can be treated with 4-nitrophenyl carbonochloridate in the presence of a tertiary amine base. Then, treated with an alcohol, R^{18b}—OH, wherein R^{18b} is optionally substituted C₁₋₆alkyl, in the presence of a base such as potassium tert-butoxide followed by removal of the PG¹ protecting group gives compounds of formula (18-8). Compounds of formula (18-7) and formula (18-8) are representative of compounds of formula (III).

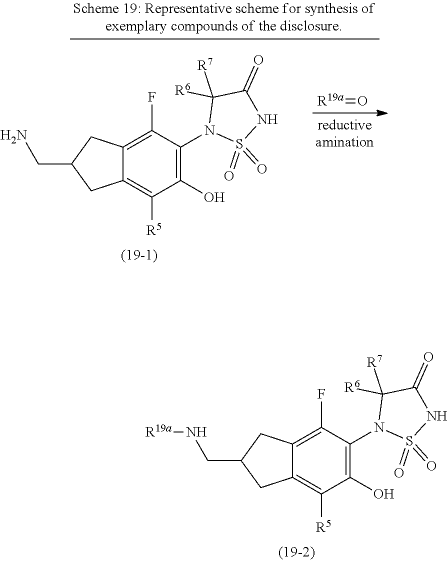

As shown in Scheme 19, compounds of formula (19-2) can be obtained from compounds of formula (19-1). Compounds of formula (19-1) can be prepared as described in the Examples. Compounds of formula (19-1) can be reductively aminated with an aldehyde or ketone (R^{19a}═O) under conditions known to one of skill in the art to give compounds of formula (19-2). R^{19a} is —C₁₋₆alkyl, —C₁₋₆alkylene-N(R^{a})—C₁₋₆alkyl, —C₁₋₆alkylene-N(R^{a})—C₁₋₆alkylene-C₃₋₆cycloalkyl, C₃₋₆cycloalkyl, —C₁₋₆alkylene-C₃₋₆cycloalkyl, or —C₁₋₆alkylene-4-6 membered heterocyclyl, wherein each moiety may be optionally substituted. Compounds of formula (19-2) are representative of compounds of formula (I).

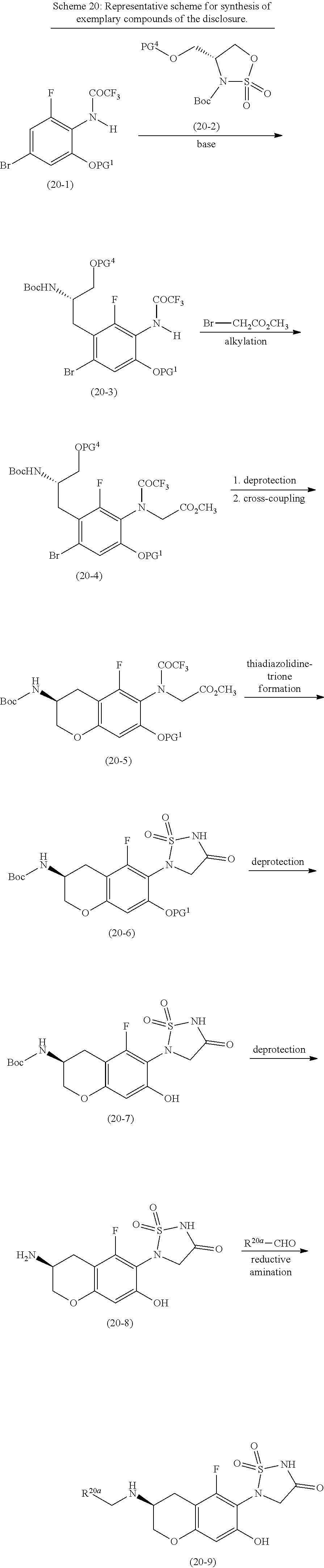

As shown in Scheme 20, compounds of formula (20-9) can be prepared from compounds of formula (20-1). Compounds of formula of formula (20-1) can be treated with a base such as lithium diisopropylamide and then reacted with a compound of formula (20-2), wherein PG⁴ is a hydroxyl protecting group, such as a silyl ether, to give compounds of formula (20-3). Compounds of formula (20-3) can be alkylated with methyl bromoacetate in the presence of a base and potassium iodide to give compounds of formula (20-4). The protecting group, PG⁴, of compounds of formula (20-4) can be selectively removed under conditions known to one of skill in the art. That intermediate can be intramolecularly cyclized under cross-coupling reaction conditions to give compounds of formula (20-5). Compounds of formula (20-5) can be treated as described in Scheme 8 to construct the thiadiazolidine-trione giving compounds of formula (20-6). The protecting groups, PG¹ and Boc, can be removed sequentially under conditions known to one of skill in the art. For example, when PG¹ is benzyl, transfer hydrogen conditions remove the benzyl group selectively to give compounds of formula (20-7). Subsequently, the tert-butoxycarbonyl protecting group can be removed by treatment under acidic conditions such as with trifluoroacetic acid in dichloromethane to give compounds of formula (20-8). Compounds of formula (20-8) can be reductively aminated with aldehydes, R^{20a}—CHO, wherein R^{20a} is optionally substituted C₁₋₈alkyl, optionally substituted-C₁₋₈alkylene-C₃₋₆cycloalkyl, or optionally substituted-C₁₋₈alkylene-4-6 membered heterocyclyl, to give compounds of formula (20-9). Compounds of formula (20-9) are representative of compounds of Formula (II).

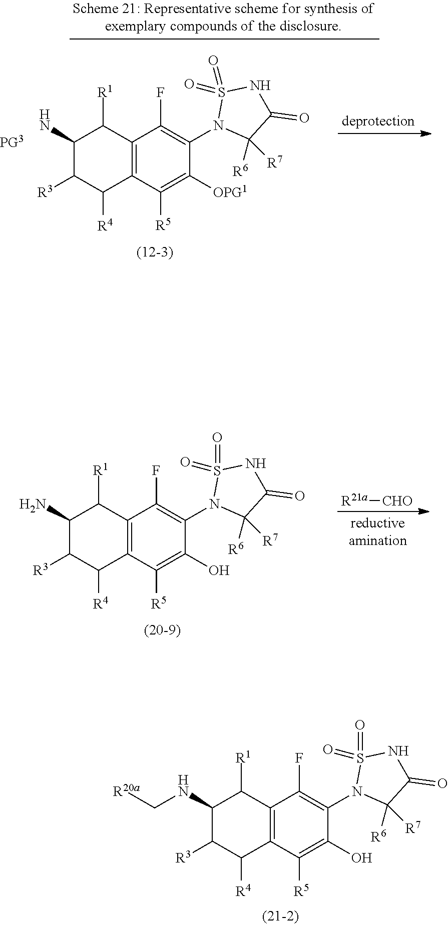

As shown in Scheme 21, compounds of formula (21-2) can be prepared from compounds of formula (12-3). Compounds of formula of formula (12-3) can be deprotected of both protecting groups, PG¹ and PG³, under conditions known to one of skill in the art. For example, when PG³ is benzyloxycarbonyl and PG¹ is benzyl, hydrogenation in the presence of a catalyst such as palladium hydroxide on carbon give compounds of formula (21-1). Compounds of formula (21-1) can be reductively aminated with aldehydes of formula R^{21a}—CHO, wherein R^{21a} is optionally substituted —C₁₋₇alkyl, optionally substituted —C₁₋₅alkylene-C₃₋₆cycloalkyl, optionally substituted —C₁₋₅alkylene-Si(R^{c})₃, optionally substituted C₁₋₅alkylene-4-6 membered heterocyclyl, optionally substituted —C₁₋₅alkylene-5-6 membered heteroaryl, or optionally substituted —C₁₋₅alkylene-phenyl to give compound of formula (21-2). Compounds of formula (21-2) are representative of compounds of Formula (I).

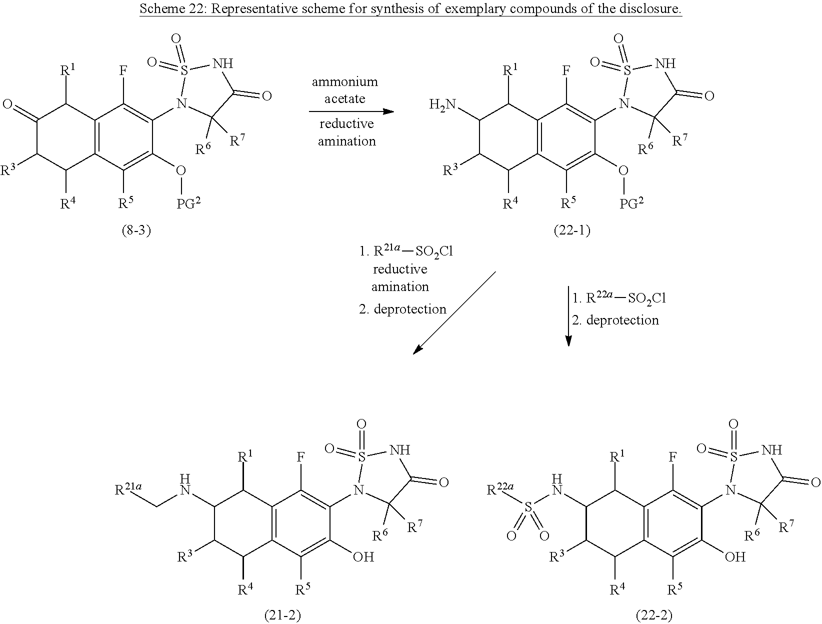

As shown in Scheme 22, compounds of formula (22-2) and compounds of formula (22-3) can be prepared from compounds of formula (8-3). Accordingly, compounds of formula of formula (8-3) can be reacted with ammonium acetate under reductive amination conditions to give compounds of formula (22-2). Compounds of formula (22-1) can be reductively aminated with aldehydes, R^{21a}—CHO, wherein R^{21a} is as described in Scheme 21, and the intermediate subsequently deprotected to give compounds of formula (21-2). Compounds of formula (22-1) can also be reacted with sulfonyl chlorides, R^{22a}—SO₂Cl, wherein R^{22a} is optionally substituted C₁₋₆alkyl, and the intermediate subsequently deprotected to give compounds of formula (22-2). Compounds of formula (21-2) and compounds of formula (22-2) are representative of compounds of Formula (I).

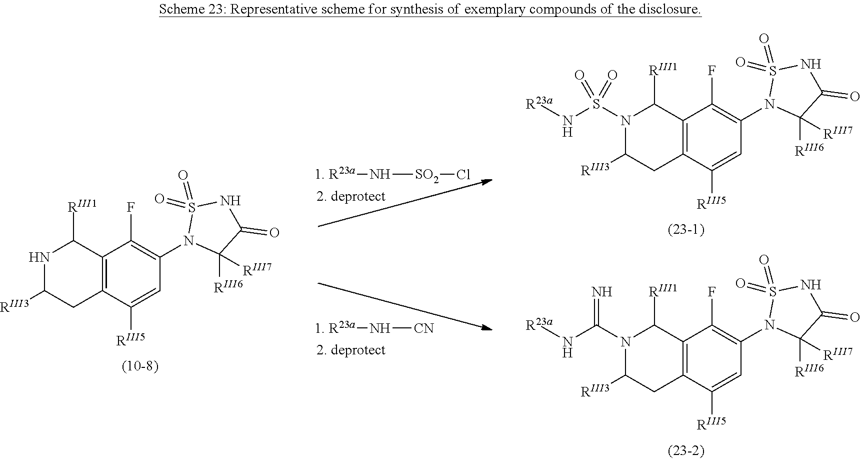

As shown in Scheme 23, compounds of formula (23-1) and compounds of formula (23-2) can be prepared from compounds of formula (10-9). Accordingly, compounds of formula (10-9) can be reacted with sulfamoyl chlorides, R^{23a}—NH—SO₂—Cl, wherein R^{23a} is an optionally substituted C₁₋₆alkyl, in the presence of a base, such as a tertiary amine base, and then subsequently deprotected under conditions known to one of skill in the art, to give compounds of formula (23-1). Alternatively, compounds of formula (10-9) can be reacted with cyanamides, R^{23a}—NH—CN, in the presence of a base, such as a tertiary amine base, and then subsequently deprotected under conditions known to one of skill in the art, to give compounds of formula (23-2). Compounds of formula (23-1) and compounds of formula (23-2) are representative of compounds of Formula (III).

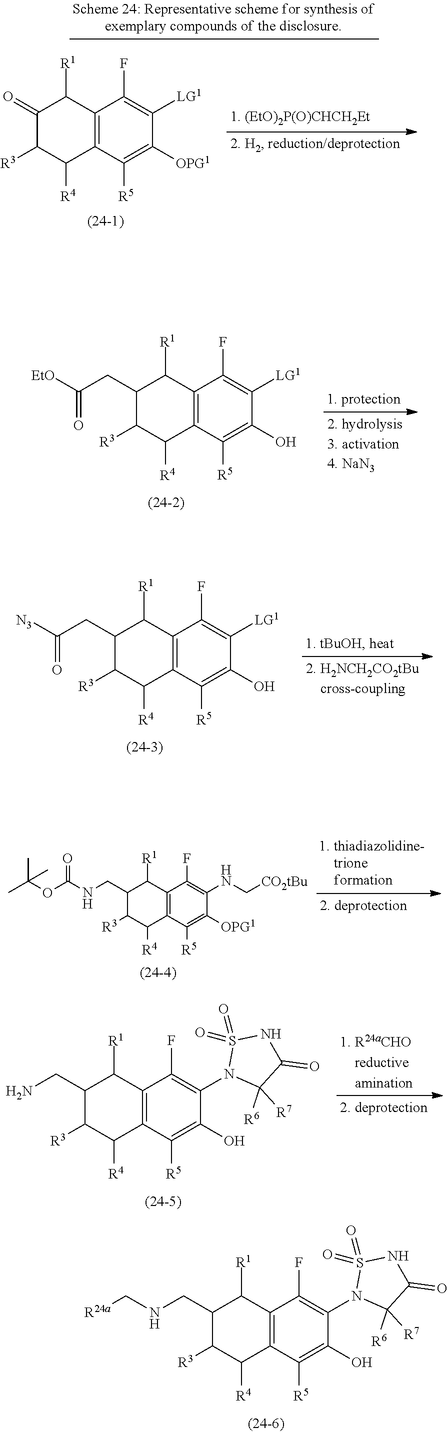

As shown in Scheme 24, compounds of formula (24-6) can be prepared from compounds of formula (24-1). Compounds of formula (24-1) can be reacted with (EtO)₂P(O)CHCO₂Et under Wittig reaction conditions and then simultaneously reduced (double bond) and deprotected to give compounds of formula (24-2). Compounds of formula (24-2) can be transformed in a four-step process to provide compounds of formula (24-3). In the first step, a phenol protecting group can be installed. In the second step, the ethyl ester can be hydrolyzed to the corresponding carboxylic acid. In the third step, the carboxylic acid moiety can be activated by reaction with ethyl chloroformate in the presence of a base such as a tertiary amine base. In the fourth step, treatment with sodium azide provides acyl azides of formula (24-3). Compounds of formula (24-3) can be treated with t-butanol under Curtius rearrangement reaction conditions to give an intermediate that is cross-coupled with tert-butyl glycine to give compound of formula (24-4). Compounds of formula (24-4) can be treated as described in Scheme 8 to construct the thiadiazolidine-trione. Subsequent amine protecting group removal gives compounds of formula (24-5). Compounds of formula (24-5) can be reductively aminated with aldehydes, R^{24a}CHO, wherein R^{24a} is —C₁₋₆alkyl or —C₁₋₆alkylene-C₃₋₆cycloalkyl, and the phenol protecting group subsequently removed to give compounds of formula (24-6). Compounds of formula (24-6) are representative of compounds of Formula (I).

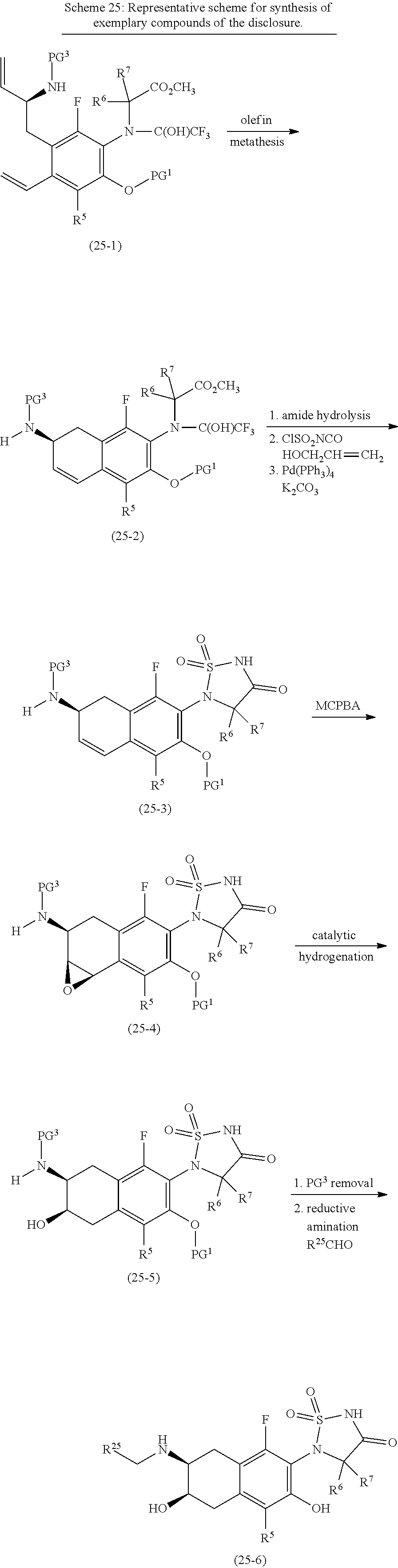

As shown in Scheme 25, compounds of formula (25-6) can be prepared from compounds of formula (25-1). Compounds of formula (25-1), wherein PG¹ is a protecting group such as benzyl and PG³ is an amine protecting group such as tert-butoxycarbonyl, can be prepared as described in the Examples or with methodology known to one of skill in the art. Compounds of formula (25-1) can be reacted under olefin metathesis reaction conditions to give compounds of formula (25-2). The trifluoroacetyl moiety of compounds of formula (25-2) can be removed under hydrolytic conditions such as with sodium methoxide in warmed methanol. Subsequent treatment with a preformed mixture of chlorosulfonyl isocyanate and allyl alcohol in a solvent such as chilled dichloromethane followed by treatment with tetrakis(triphenylphosphine)palladium(0) in the presence of a base such as potassium carbonate can give the corresponding 1λ⁶,2,5-thiadiazolidine-1,1,3-trione moiety of compounds of formula (25-3). Compounds of formula (25-3) can be epoxidized to compounds of formula (25-4) upon treatment with 3-chloroperoxybenzoic acid in the presence of sodium bicarbonate in chilled dichloromethane. Treatment of compounds of formula (25-4) under catalytic hydrogenation conditions can remove the protecting group, PG¹, when PG¹ is benzyl and simultaneously open the epoxide ring to give compounds of formula (25-5). When PG³ is tert-butoxycarbonyl, treatment with an acid such as trifluoroacetic acid in dichloromethane is suitable for protecting group removal. Subsequent reductive amination with aldehydes, R²⁵CHO, give compounds of formula (25-6). Compounds of formula (25-6) are representative of compounds of Formula (I).

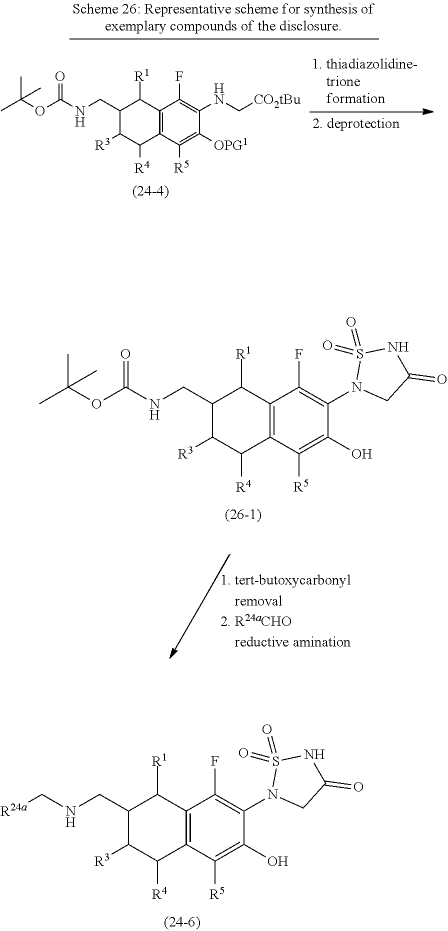

As shown in Scheme 26, compounds of formula (24-6) can also be prepared from compounds of formula (24-4) in an alternative synthetic sequence. Compounds of formula (24-4) can be treated as described in Scheme 8 to construct the thiadiazolidine-trione. Subsequent removal of protecting group PG¹, under transfer hydrogenation conditions when PG¹ is benzyl, gives compounds of formula (26-1). The tert-butoxycarbonyl protecting group of compounds of formula (26-1) can be removed under acidic conditions known to one of skill in the art such as trifluoracetic acid in dichloromethane. Subsequent reductive amination with aldehydes, R^{24a}CHO, wherein R^{24a} is —C₁₋₆alkyl or —C₁₋₆alkylene-C₃₋₆cycloalkyl, give compounds of formula (24-6). Compounds of formula (24-6) are representative of compounds of Formula (I).

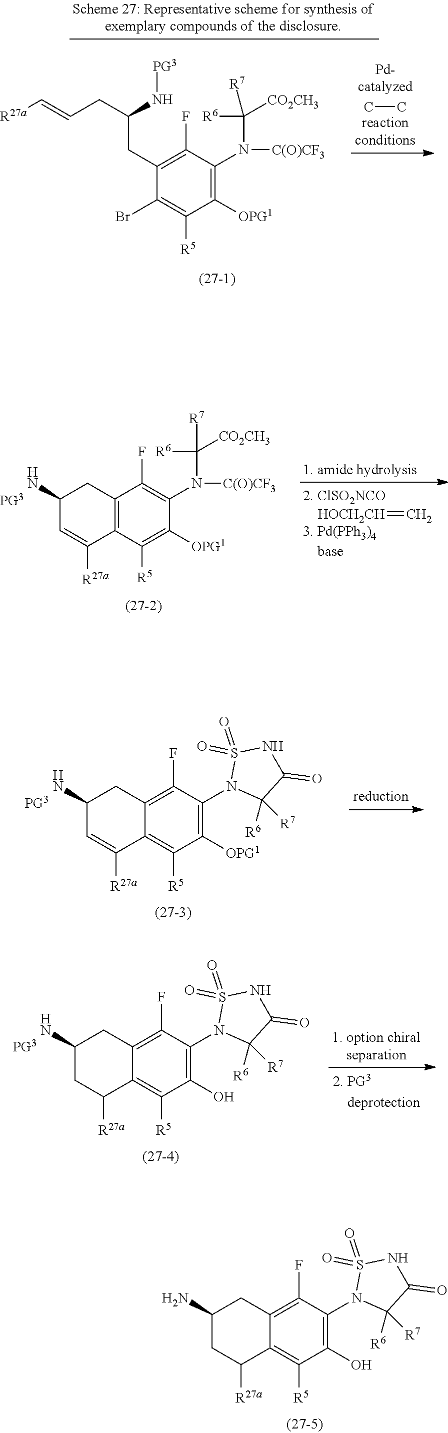

As shown in Scheme 27, compounds of formula (27-5) can be prepared from compounds of formula (27-1). Compounds of formula (27-1), wherein PG¹ is a protecting group such as benzyl and PG³ is an amine protecting group such as tert-butoxycarbonyl, can be prepared as described in the Examples. Compounds of formula (27-1) can be cyclized to compounds of formula (27-1) under palladium-catalyzed under C-C cross-coupling reaction conditions such as Heck reaction conditions to give compounds of formula (27-2). The trifluoroacetyl moiety of compounds of formula (27-2) can be removed under hydrolytic conditions such as with sodium methoxide in warmed methanol. Subsequent treatment with a preformed mixture of chlorosulfonyl isocyanate and allyl alcohol in a solvent such as chilled dichloromethane followed by treatment with tetrakis(triphenylphosphine)palladium(0) in the presence of a base such as potassium carbonate, sodium tert-butoxide, or sodium methoxide can give the corresponding 1λ⁶,2,5-thiadiazolidine-1,1,3-trione moiety of compounds of formula (27-3). Catalytic transfer hydrogenation can both reduce the double bond in compounds of formula (27-3) while simultaneously removing the protecting group, PG¹, when PG¹ is a protecting group such as benzyl to give compounds of formula (27-4). Compounds of formula (27-4) can be separated into the respective enantiomers by chiral chromatography. The protecting group, PG³, can be removed under conditions known to one of skill in the art such as acidic conditions (trifluoroacetic acid in dichloromethane or hydrochloric acid in dioxane) when PG³ is an amine protecting group such as tert-butoxycarbonyl to give compounds of formula (27-5). Compounds of formula (27-4) and formula (27-5) are representative of compounds of Formula (I).

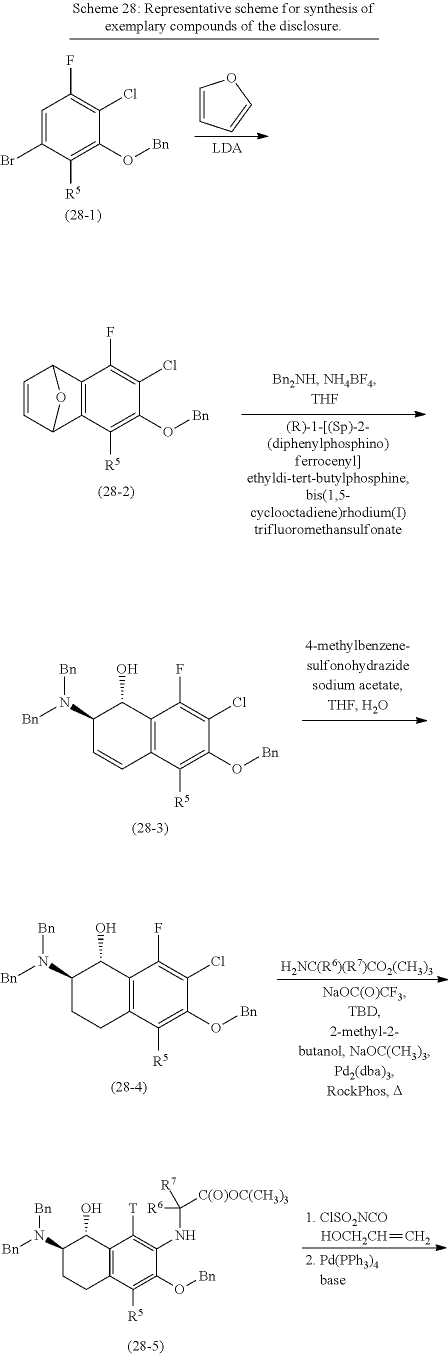

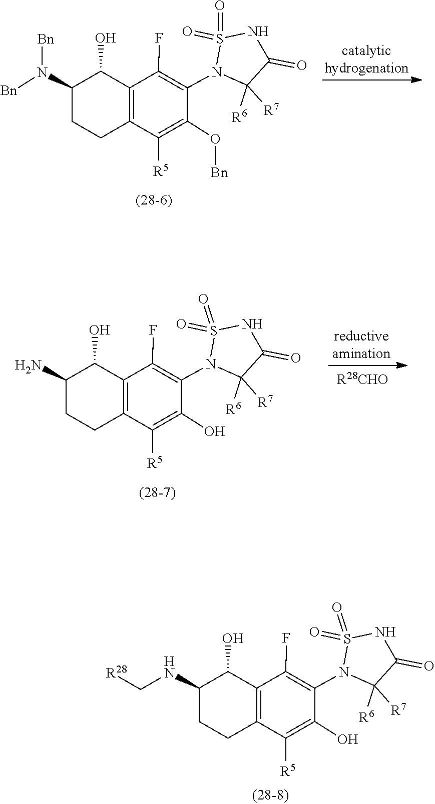

As shown in Scheme 28, compounds of formula (28-8) can be prepared from compounds of formula (28-1). Compounds of formula (28-1), wherein Bn is benzyl, can be reacted with cooled furan in the presence of lithium diisopropylamide to give compounds of formula (28-2). Compounds of formula (28-2) can be reacted with dibenzylamine in the presence of ammonium tetrafluoroborate, (R)-1-[(Sp)-2-(diphenylphosphino)ferrocenyl]ethyldi-tert-butylphosphine, and bis(1,5-cyclooctadiene) rhodium (I) trifluoromethanesulfonate in warmed tetrahydrofuran to give compounds of formula (28-3). Compounds of formula can be separated to the respective enantiomers using chiral chromatography. Reduction of the double bond in compounds of formula (28-3) to give compounds of formula (28-4) can be achieved by treatment with 4-methylbenzenesulfonohydrazide and sodium acetate in a mixture of warmed water and tetrahydrofuran. Coupling of the amino ester, H₂NC(R⁶)(R⁷)CO₂C(CH₃)₃, with compounds of formula (28-4) is carried out in a solvent such as 2-methyl-2-butanol in the presence of 1,5,7-triazabicyclo[4.4.0]dec-5-ene and sodium trifluoroacetate, and a catalyst mixture of a base such as sodium tert-butoxide, a palladium catalyst such as tris(dibenzylideneacetone)dipalladium(0), a ligand such as RockPhos to give compounds of formula (28-5). Treatment with a preformed mixture of chlorosulfonyl isocyanate and allyl alcohol in a solvent such as chilled dichloromethane in the presence of a base such as N,N-diisopropylethylamine followed by treatment with tetrakis(triphenylphosphine)palladium(0) in heated methanol in the presence of a base such as potassium tert-butoxide, potassium carbonate or sodium methoxide can give the corresponding 1λ⁶,2,5-thiadiazolidine-1,1,3-trione moiety of compounds of formula (28-6). The benzyl groups of compounds of formula (28-6) can be removed under catalytic hydrogenation conditions to give compounds of formula (28-7). Reductive amination of compounds of formula (28-7) with aldehydes, R²⁸CHO; where R²⁸ is —C₁₋₅alkyl, —C₂₋₅alkenyl, —C₁₋₅alkylene-N(R^{a})—C₁₋₆alkyl, —C₁₋₅alkylene-N(R^{a})—C₁₋₆alkylene-C₃₋₆cycloalkyl, —C₁₋₅alkylene-N(R^{a})(R^{b}), —C₁₋₅alkylene-N(R^{a})—C(O)—O—C₁₋₆alkyl, —C₁₋₅alkylene-C₃₋₆cycloalkyl, and —C₁₋₅alkylene-4-6 membered heterocyclyl; can give compounds of formula (28-8). Compounds of formula (28-7) and compounds of formula (28-7) are representative of compounds of Formula (I).

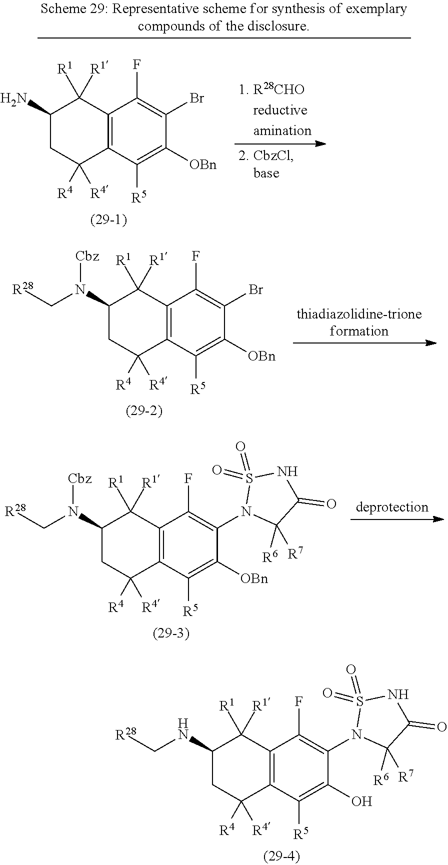

As shown in Scheme 29, compounds of formula (29-4) can be prepared from compounds of formula (29-1). Compounds of formula (29-1), wherein Bn is benzyl can be prepared as described in the Examples. Compounds of formula (29-1) can be transformed to compounds of formula (29-2) by reductive amination with aldehydes, R²⁸CHO, followed by protection of the amine as for example the benzyloxycarbonyl (Cbz) carbamate. Compounds of formula (29-2) can be converted to compounds of formula (29-3) by using the synthetic methodology described in Scheme 28 for the conversion of compounds of formula (28-4) to compound of formula (28-6). Removal of both the benzyl and benzyloxycarbonyl moieties of compound of formula (29-3) can be achieved under catalytic hydrogenation conditions to give compounds of formula (29-4). Compounds of formula (29-4) are representative of compound of Formula (I).

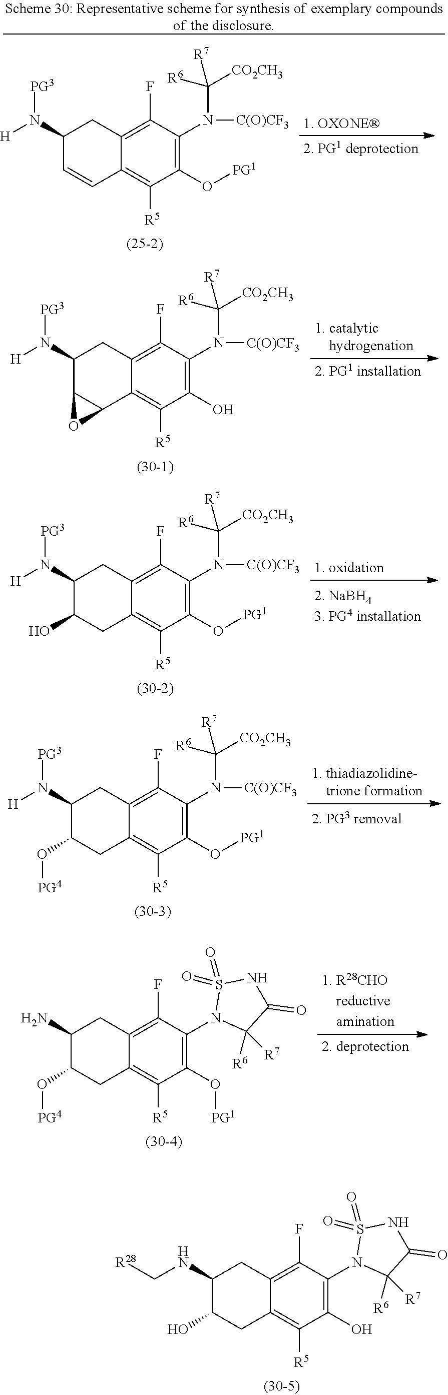

As shown in Scheme 30, compounds of formula (30-5) can be prepared from compounds of formula (25-2). Compounds of formula (25-2), wherein PG¹ is a protecting group such as benzyl and PG³ is an amine protecting group such as tert-butoxy carbonyl, can be oxidized with potassium peroxomonosulfate (OXONE®) in the presence of sodium bicarbonate in a chilled mixture of water and ethyl acetate to give the corresponding epoxide. Subsequent removal of PG¹ under catalytic hydrogenation conditions give compounds of formula (30-1). Compounds of (30-1) can also be treated under catalytic hydrogenation conditions to open the epoxide. Reinstallation of PG¹, such as treatment with benzyl bromide in the presence of a base such as potassium carbonate in a solvent such as N,N-dimethyformamide, delivers compounds of formula (30-2). Compounds of formula (30-2) can be oxidized to the corresponding ketone with for example Dess-Martin periodinane. Reduction of the ketone with a reductant such as sodium borohydride gives the inverted alcohol. Protection of the newly formed alcohol as a silyl ether can be achieved by treatment with tert-butyldimethylchlorosilane in the presence of imidazole in N,N-dimethylformamide to give compounds of formula (30-3). Compounds of formula (30-3) can be converted to the corresponding 1λ⁶,2,5-thiadiazolidine-1,1,3-trione by using the synthetic methodology described in Scheme 27 for the conversion of compounds of formula (27-2) to compound of formula (27-3). Removal of the PG³, typically a tert-butoxycarbonyl, can be achieved by treatment with an acid such as trifluoroacetic acid in dichloromethane or hydrochloric acid in dioxane gives compounds of formula (30-4). Compounds of formula (30-4) can be reductively aminated with aldehydes, R²⁸CHO. Subsequent removal of PG¹ using catalytic hydrogenation followed by treatment with acetic acid in a mixture of water and tetrahydrofuran to remove PG⁴ gives compounds of formula (30-5). Compounds of formula (30-5) are representative of compounds of Formula (I).

### Pharmaceutical Compositions

The present disclosure provides pharmaceutical compositions comprising a compound disclosed herein, e.g., a compound of Formula (I), Formula (II) or Formula (III). In some embodiments, the pharmaceutical composition further comprises a pharmaceutically acceptable excipient. In some embodiments, a compound disclosed herein, e.g., a compound of Formula (I), Formula (II) or Formula (III), is provided in an effective amount in the pharmaceutical composition. In some embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing a disclosed compound (the “active ingredient”) into association with a carrier and/or one or more other accessory ingredients, and then, if necessary and/or desirable, shaping and/or packaging the product into a desired single- or multi-dose unit. Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of a compound disclosed herein, e.g., a compound of Formula (I). Formula (II) or Formula (III), the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) of a compound disclosed herein.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the disclosure are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present disclosure may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water. Ringer's solution and isotonic sodium chloride solution. In addition, sterile, fixed oils are conventionally employed as a solvent or suspending medium.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A compound disclosed herein may also be in micro-encapsulated form.

The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao. J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles. J. Pharm. Pharmacol. 49:669-674, 1997). In another embodiment, the formulations of the compositions of the present disclosure can be delivered by the use of liposomes which fuse with the cellular membrane or are endocytosed, e.g., by employing receptor ligands attached to the liposome that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, J. Hosp. Pharm. 46:1576-1587, 1989). The compositions of the present disclosure can also be delivered as nanoparticles.

Alternatively, pharmaceutically acceptable compositions of the present disclosure may be administered in the form of suppositories for rectal administration. Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

In some embodiments, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein, e.g., a compound of Formula (I), Formula (II) or Formula (III) are typically formulated in dosage unit form, e.g., single unit dosage form, for ease of administration and uniformity of dosage. It will be understood, however, that the total daily usage of the compositions of the present disclosure will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition disclosed herein can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and pain-relieving agents. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids. DNAs. RNAs, nucleotides, nucleosides, oligonucleotides, antisense oligonucleotides, lipids, hormones, vitamins, and cells.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., inhibiting the activity of a target molecule (e.g. PTPN2 and/or PTPN1), and/or reducing, eliminating, or slowing the progression of disease symptoms. Determination of a therapeutically effective amount of a compound disclosed herein is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods, compounds and compositions disclosed herein. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

Also encompassed by the present are kits (e.g., pharmaceutical packs). The kits provided herein may be useful for preventing and/or treating a disease (e.g., cancer, type-2 diabetes, obesity, a metabolic disease, or other disease or condition described herein).

The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one unit dosage form.

Thus, in one aspect, provided are kits including a first container comprising a compound disclosed herein. In certain embodiments, the kits are useful in preventing and/or treating a proliferative disease in a subject. In certain embodiments, the kits further include instructions for administering a disclosed compound to a subject to prevent and/or treat a disease described herein.

### Methods of Treatment

The present disclosure features compounds, compositions, and methods comprising a compound disclosed herein, e.g., a compound of Formula (I), Formula (II) or Formula (III). In some embodiments, the compounds, compositions, and methods disclosed herein are used in the prevention or treatment of a disease, disorder, or condition. Exemplary diseases, disorders, or conditions include, but are not limited to cancer, type-2 diabetes, metabolic syndrome, obesity, or a metabolic disease.

### Cancer

In some embodiments, a compound disclosed herein, e.g., a compound of Formula (I), Formula (II) or Formula (III) is used to treat cancer. As used herein, “cancer” refers to human cancers and carcinomas, sarcomas, adenocarcinomas, lymphomas, leukemias, melanomas, etc., including solid and lymphoid cancers, kidney, breast, lung, bladder, colon, ovarian, prostate, pancreas, stomach, brain, head and neck, skin, uterine, testicular, glioma, esophagus, liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), and/or multiple myeloma. In some further instances, “cancer” refers to lung cancer, breast cancer, ovarian cancer, leukemia, lymphoma, melanoma, pancreatic cancer, sarcoma, bladder cancer, bone cancer, brain cancer, cervical cancer, colon cancer, esophageal cancer, gastric cancer, liver cancer, head and neck cancer, kidney cancer, myeloma, thyroid cancer, prostate cancer, metastatic cancer, or carcinoma.

As used herein, the term “cancer” refers to all types of cancer, neoplasm or malignant tumors found in mammals, including leukemia, lymphoma, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., ER positive. ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, or melanoma. Additional examples include, cancer of the thyroid, endocrine system, brain, breast, cervix, colon, head & neck, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma. Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma. Ewing's sarcoma, fascial sarcoma, fibroblastic sarcoma, giant cell sarcoma, granulocytic sarcoma. Hodgkin's sarcoma, idiopathic multiple pigmented hemorrhagic sarcoma, immunoblastic sarcoma of B cells, lymphoma, immunoblastic sarcoma of T-cells, Jensen's sarcoma. Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma. Cloudman's melanoma. S91 melanoma. Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma. Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma. Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

In some embodiments, a compound disclosed herein, e.g., a compound of Formula (I), Formula (II) or Formula (III), is used to treat pancreatic cancer, breast cancer, multiple myeloma, cancers of secretory cells. For example certain methods herein treat cancer by decreasing or reducing or preventing the occurrence, growth, metastasis, or progression of cancer. In some embodiments, the methods described herein may be used to treat cancer by decreasing or eliminating a symptom of cancer. In some embodiments, a compound disclosed herein, e.g., a compound of Formula (I), Formula (II) or Formula (III), may be used as a single agent in a composition or in combination with another agent in a composition to treat a cancer described herein (e.g., pancreatic cancer, breast cancer, multiple myeloma, cancers of secretory cells).

In some embodiments, the compounds (compounds described herein, e.g., a compound of Formula (I), Formula (II) or Formula (III)) and compositions (e.g., compositions comprising a compound described herein, e.g., a compound of Formula (I), Formula (II) or Formula (III)) are used with a cancer immunotherapy (e.g., a checkpoint blocking antibody) to treat a subject (e.g., a human subject), e.g., suffering from a disease or disorder described herein (e.g., abnormal cell growth, e.g., cancer (e.g., a cancer described herein)). The methods described herein comprise administering a compound described herein, e.g., a compound of Formula (I), Formula (II) or Formula (III) and an immunotherapy to a subject having abnormal cell growth such as cancer. Exemplary immunotherapies include, but are not limited to the following.

In some embodiments, the immunotherapeutic agent is a compound (e.g., a ligand, an antibody) that inhibits the immune checkpoint blockade pathway. In some embodiments, the immunotherapeutic agent is a compound that inhibits the indoleamine 2,3-dioxygenase (IDO) pathway. In some embodiments, the immunotherapeutic agent is a compound that agonizes the STING pathway. Cancer immunotherapy refers to the use of the immune system to treat cancer. Three groups of immunotherapy used to treat cancer include cell-based, antibody-based, and cytokine therapies. All groups exploit cancer cells” display of subtly different structures (e.g., molecular structure; antigens, proteins, molecules, carbohydrates) on their surface that can be detected by the immune system. Cancer immunotherapy (e.g., anti-tumor immunotherapy or anti-tumor immunotherapeutics) includes but is not limited to, immune checkpoint antibodies (e.g., PD-1 antibodies, PD-L1 antibodies, PD-L2 antibodies, CTLA-4 antibodies, TIM3 antibodies, LAG3 antibodies, TIGIT antibodies); and cancer vaccines (e.g., anti-tumor vaccines or vaccines based on neoantigens such as a peptide or RNA vaccine).

Cell-based therapies (e.g., cancer vaccines), usually involve the removal of immune cells from a subject suffering from cancer, either from the blood or from a tumor. Immune cells specific for the tumor will be activated, grown, and returned to a subject suffering from cancer where the immune cells provide an immune response against the cancer. Cell types that can be used in this way are e.g., natural killer cells, lymphokine-activated killer cells, cytotoxic T-cells, dendritic cells, CAR-T therapies (e.g., chimeric antigen receptor T-cells which are T-cells engineered to target specific antigens), TIL therapy (e.g., administration of tumor-infiltrating lymphocytes), TCR gene therapy, protein vaccines, and nucleic acid vaccines. An exemplary cell-based therapy is Provenge. In some embodiments, the cell-based therapy is a CAR-T therapy.

Interleukin-2 and interferon-alpha are examples of cytokines, proteins that regulate and coordinate the behavior of the immune system.

Cancer Vaccines with Neoantigens

Neoantigens are antigens encoded by tumor-specific mutated genes. Technological innovations have made it possible to dissect the immune response to patient-specific neoantigens that arise as a consequence of tumor-specific mutations, and emerging data suggest that recognition of such neoantigens is a major factor in the activity of clinical immunotherapies. These observations indicate that neoantigen load may form a biomarker in cancer immunotherapy. Many novel therapeutic approaches are being developed that selectively enhance T cell reactivity against this class of antigens. One approach to target neoantigens is via cancer vaccine. These vaccines can be developed using peptides or RNA, e.g., synthetic peptides or synthetic RNA.

Antibody therapies are antibody proteins produced by the immune system and that bind to a target antigen on the surface of a cell. Antibodies are typically encoded by an immunoglobulin gene or genes, or fragments thereof. In normal physiology antibodies are used by the immune system to fight pathogens. Each antibody is specific to one or a few proteins, and those that bind to cancer antigens are used, e.g., for the treatment of cancer. Antibodies are capable of specifically binding an antigen or epitope (Fundamental Immunology, 3^{rd} Edition, Paul. W. E, ed., Raven Press, N.Y. (1993). Specific binding occurs to the corresponding antigen or epitope even in the presence of a heterogeneous population of proteins and other biologics. Specific binding of an antibody indicates that it binds to its target antigen or epitope with an affinity that is substantially greater than binding to irrelevant antigens. The relative difference in affinity is often at least 25% greater, more often at least 50% greater, most often at least 100% greater. The relative difference can be at least 2-fold, at least 5-fold, at least 10-fold, at least 25-fold, at least 50-fold, at least 100-fold, or at least 1000-fold, for example.

Exemplary types of antibodies include without limitation human, humanized, chimeric, monoclonal, polyclonal, single chain, antibody binding fragments, and diabodies. Once bound to a cancer antigen, antibodies can induce antibody-dependent cell-mediated cytotoxicity, activate the complement system, prevent a receptor interacting with its ligand or deliver a payload of chemotherapy or radiation, all of which can lead to cell death. Exemplary antibodies for the treatment of cancer include but are not limited to, Alemtuzumab, Bevacizumab, Bretuximab vedotin, Cetuximab, Gemtuzumab ozogamicin, Ibritumomab tiuxetan, Ipilimumab, Ofatumumab, Panitumumab, Rituximab, Tositumomab, Trastuzumab, Nivolumab, Pembrolizumab, Avelumab, durvalumab and pidilizumab.

### Checkpoint Blocking Antibodies

The methods described herein comprise, in some embodiments, treating a human subject suffering from a disease or disorder described herein, the method comprising administering a composition comprising a cancer immunotherapy (e.g., an immunotherapeutic agent). In some embodiments, the immunotherapeutic agent is a compound (e.g., an inhibitor or antibody) that inhibits the immune checkpoint blockade pathway. Immune checkpoint proteins, under normal physiological conditions, maintain self-tolerance (e.g., prevent autoimmunity) and protect tissues from damage when the immune system is responding to e.g., pathogenic infection. Immune checkpoint proteins can be dysregulated by tumors as an important immune resistance mechanism (Pardoll, Nature Rev. Cancer. 2012, 12, 252-264). Agonists of co-stimulatory receptors or antagonists of inhibitory signals (e.g., immune checkpoint proteins), provide an amplification of antigen-specific T-cell responses. Antibodies that block immune checkpoints do not target tumor cells directly but typically target lymphocyte receptors or their ligands to enhance endogenous antitumor activity.

Exemplary checkpoint blocking antibodies include but are not limited to, anti-CTLA-4, anti-PD-1, anti-LAG3 (e.g., antibodies against lymphocyte activation gene 3), and anti-TIM3 (e.g., antibodies against T-cell membrane protein 3). Exemplary anti-CTLA-4 antibodies include but are not limited to, ipilimumab and tremelimumab. Exemplary anti-PD-1 ligands include but are not limited to, PD-L1 (e.g., B7-H1 and CD274) and PD-L2 (e.g., B7-DC and CD273). Exemplary anti-PD-1 antibodies include but are not limited to, nivolumab (e.g., MDX-1106, BMS-936558, or ONO-4538)), CT-011, AMP-224, pembrolizumab (trade name Keytruda), and MK-3475. Exemplary PD-L1-specific antibodies include but are not limited to, BMS936559 (e.g., MDX-1105), MEDI4736 and MPDL-3280A. Exemplary checkpoint blocking antibodies also include but are not limited to, IMP321 and MGA271.

T-regulatory cells (e.g., CD4+, CD25+, or T-reg) are also involved in policing the distinction between self and non-self (e.g., foreign) antigens, and may represent an important mechanism in suppression of immune response in many cancers. T-reg cells can either emerge from the thymus (e.g., “natural T-reg”) or can differentiate from mature T-cells under circumstances of peripheral tolerance induction (e.g., “induced T-reg”). Strategies that minimize the action of T-reg cells would therefore be expected to facilitate the immune response to tumors.

### IDO Pathway Inhibitors

The IDO pathway regulates immune response by suppressing T cell function and enabling local tumor immune escape. IDO expression by antigen-presenting cells (APCs) can lead to tryptophan depletion, and resulting antigen-specific T cell energy and regulatory T cell recruitment. Some tumors even express IDO to shield themselves from the immune system. A compound that inhibits IDO or the IDO pathway activates the immune system to attack the cancer (e.g., tumor in a subject). Exemplary IDO pathway inhibitors include indoximod, epacadostat and EOS200271.

### STING Pathway Agonists

Stimulator of interferon genes (STING) is an adaptor protein that plays an important role in the activation of type I interferons in response to cytosolic nucleic acid ligands. Evidence indicates involvement of the STING pathway in the induction of antitumor immune response. For example, activation of the STING-dependent pathway in cancer cells can result in tumor infiltration with immune cells and modulation of the anticancer immune response. STING agonists are being developed as a class of cancer therapeutics. Exemplary STING agonists include MK-1454 and ADU-S100.

### Co-Stimulatory Antibodies

The methods described herein comprise, in some embodiments, treating a human subject suffering from a disease or disorder described herein, the method comprising administering a composition comprising a cancer immunotherapy (e.g., an immunotherapeutic agent). In some embodiments, the immunotherapeutic agent is a co-stimulatory inhibitor or antibody. In some embodiments, the methods described herein comprise depleting or activating anti-4-1BB, anti-OX40, anti-GITR, anti-CD27 and anti-CD40, and variants thereof.

Methods of the present disclosure contemplate single as well as multiple administrations of a therapeutically effective amount of a compound as described herein. Compounds, e.g., a compound as described herein, can be administered at regular intervals, depending on the nature, severity and extent of the subject's condition. In some embodiments, a compound described herein is administered in a single dose. In some embodiments, a compound described herein is administered in multiple doses.

### Metabolic Diseases

In some embodiments, a compound disclosed herein, e.g., a compound of Formula (I), Formula (II) or Formula (III), is used to treat metabolic disease. As used herein, the term “metabolic disease” refers to a disease or condition affecting a metabolic process in a subject. Exemplary metabolic diseases that may be treated with a compound disclosed herein, e.g., a compound of Formula (I), Formula (II) or Formula (III), include non-alcoholic steatohepatitis (NASH), non-alcoholic fatty liver disease (NAFLD), liver fibrosis, obesity, heart disease, atherosclerosis, arthritis, cystinosis, diabetes (e.g., Type I diabetes, Type II diabetes, or gestational diabetes), metabolic syndrome, phenylketonuria, proliferative retinopathy, or Kearns-Sayre disease.

In some embodiments, a compound disclosed herein, e.g., a compound of Formula (I), Formula (II) or Formula (III), is used to treat a metabolic disease (e.g., a metabolic disease described herein) by decreasing or eliminating a symptom of the disease. In some embodiments, the method of treatment comprises decreasing or eliminating a symptom comprising elevated blood pressure, elevated blood sugar level, weight gain, fatigue, blurred vision, abdominal pain, flatulence, constipation, diarrhea, jaundice, and the like. In some embodiments, a compound disclosed herein, e.g., a compound of Formula (I), Formula (II) or Formula (III), may be used as a single agent in a composition or in combination with another agent in a composition to treat a metabolic disease.

In some embodiments, the compounds disclosed herein are provided as pharmaceutical compositions including a disclosed compound, e.g., of Formula (I), Formula (II) or Formula (III) and a pharmaceutically acceptable excipient. In embodiments of the method, a disclosed compound, e.g., of Formula (I), Formula (II) or Formula (III) is co-administered with a second agent (e.g., therapeutic agent). In other embodiments of the method, a disclosed compound, e.g., of Formula (I), Formula (II) or Formula (III) is co-administered with a second agent (e.g., therapeutic agent), which is administered in a therapeutically effective amount.

### Combination Therapy

The present disclosure provides a pharmaceutical composition comprising a compound disclosed herein, e.g., a compound of Formula (I), Formula (II) or Formula (III), as well as a second agent (e.g., a second therapeutic agent). In some embodiments, the pharmaceutical composition includes a second agent (e.g., a second therapeutic agent) in a therapeutically effective amount. In some embodiments, the second agent is an agent for treating cancer, a metabolic disease (e.g., type-2 diabetes or obesity) or a disease or disorder favorably responsive to PTPN2 or PTPN1 inhibitor treatment.

The compounds described herein can be used in combination with one another, with other active agents known to be useful in treating cancer, a metabolic disease (e.g., type-2 diabetes or obesity) or a disease or disorder favorably responsive to PTPN2 or PTPN1 inhibitor treatment, or with adjunctive agents that may not be effective alone but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for a cancer, a metabolic disease (e.g., type-2 diabetes or obesity) or a disease or disorder favorably responsive to PTPN2 or PTPN1 inhibitor treatment. In embodiments, the second agent is an anti-cancer agent. In embodiments, the second agent is a chemotherapeutic. In embodiments, the second agent is an agent for treating a metabolic disease. In embodiments, the second agent is an anti-diabetic agent. In some embodiments, the second agent is an anti-obesity agent.

### Anti-Cancer Agents

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anticancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine. Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP 16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 1 1-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide: kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn: 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rlL.sub.2), interferon alfa-2a; interferon alfa-2b; interferon alfa-n1; interferon alfa-n3; interferon beta-la; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules. (e.g. Taxol, i.e. paclitaxel), Taxotere, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and SC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-1 12378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39·HCl), AC-7700) (Ajinomoto, i.e., AVE-8062, AVE-8062A, CS-39-L-Ser·HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A 1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, Inanocine (i.e., NSC-698666), 3-IAABE (Cytoskeleton/Mt. Sinai School of Medicine), A-204197 (Abbott), T-607 (Tularik, i.e., T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta medica), D-68144 (Asta medica), Diazonamide A, A-293620) (Abbott), NPI-2350) (Nereus), Taccalonolide A, TUB-245 (Aventis), A-259754 (Abbott), Diozostatin, (−)-Phenylahistin (i.e., NSCL-96F037), D-68838 (Asta medica), D-68836 (Asta medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-25041 1 (Sanofi), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD₂O monoclonal antibody conjugated to ^{U 1}ln, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib. EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Guerin (BCG), levamisole, interleukin-2, alpha-interferon, etc.), monoclonal antibodies (e.g., anti-CD₂O, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD₂O) monoclonal antibody conjugated to ^{m}In, ⁹⁰Y, or ¹³¹I, etc.).

In a further embodiment, the compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ^{m}Ag, ^{m}In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Synthetic Protocols

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures. General schemes relating to methods of making exemplary compounds of the invention are additionally described in the section entitled Methods of Making Exemplary Compounds.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Abbreviations

ABPR for automated back pressure regulator; AcOH or HOAc for acetic acid; APCI for atmospheric pressure chemical ionization; 9-BBN for 9-borabicyclo[3.3.1]nonane; Bn for benzyl; BrettPhos for 2-(dicyclohexylphosphino) 3,6-dimethoxy-2′,4′,6′-triisopropyl-1,1′-biphenyl; BrettPhos Pd G3 precatalyst for [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate: 1-Bu for tert-butyl; t-BuBrettPhos Pd G3 precatalyst for 2-(di-tert-butylphosphino)-2′,4′,6′-triisopropyl-3,6-dimethoxy-1,1′-biphenyl)-2-(2′-amino-1,1′-biphenyl)]palladium(II) methanesulfonate; dba for dibenzylideneacetone; DCM for dichloromethane; DMF for N,N-dimethylformamide; DMSO for dimethyl sulfoxide; ee for enantiomeric excess: ESI for electrospray ionization; Et for ethyl; HPLC for high performance liquid chromatography; i.d. for internal diameter: MCPBA for 3-chloroperoxy benzoic acid; MS for mass spectrum: MP for macroporous; NMR for nuclear magnetic resonance; Ph for phenyl; ppm for parts per million; psi for pounds per square inch; PTFE for polytetrafluoroethylene; RockPhos for 2-di(tert-butyl)phosphino-2′,4′,6′-triisopropyl-3-methoxy-6-methylbiphenyl; RockPhos Pd G3 precatalyst for [(2-di-tert-butylphosphino-3-methoxy-6-methyl-2′,4′,6′-triisopropyl-1,1′-biphenyl)-2-(2-aminobiphenyl)]palladium(II) methanesulfonate; SFC for supercritical fluid chromatography; TBD for 1,5,7-triazabicyclo[4.4.0]dec-5-ene; TCDI for 1,1′-thiocarbonyldiimidazole; THF for tetrahydrofuran; TLC for thin layer chromatography; UV for ultraviolet; v/v for volume/volume; w/v for weight/volume; and w/w for weight/weight.

### Example 1: 5-(5-fluoro-7-hydroxy-3,4-dihydro-2H-1-benzopyran-6-yl)-1λ⁶,2,5-thiadiazolidine-1,1,3-trione (Compound 111)

### Example 1A: 1-(benzyloxy)-5-bromo-3-fluoro-2-nitrobenzene

To a suspension of 5-bromo-1,3-difluoro-2-nitrobenzene (40 g, 168 mmol) and benzyl alcohol (18.4 mL, 176 mmol) in tetrahydrofuran (800 mL) at −60° C. was added a solution of potassium tert-butoxide (176 mL, 176 mmol, 1 M in tetrahydrofuran) slowly along the side of the flask so that the internal temperature remained below −50° C. After complete addition, the mixture was stirred for 5 minutes, then was quenched with saturated aqueous ammonium chloride (40 mL), diluted with water (200 mL) and ethyl acetate (200 mL) and warmed to room temperature. The aqueous layer was extracted with ethyl acetate (200 mL). The combined organic fractions were washed with brine (160 mL), dried over anhydrous sodium sulfate, filtered and concentrated in vacuo to give a solid. Heptanes (500 mL) were added to the crude solid, the mixture was heated to an internal temperature of 65° C., then slowly cooled to room temperature, and the solids were collected by filtration. The solids were washed with the cold mother liquor and additional heptane (120 mL) and then were dried in a vacuum oven at 60° C. to constant weight to give 39.95 g of the title compound. The mother liquor was concentrated and then solids were precipitated from heptanes (100 mL) to give an additional 7.56 g of the title compound. Total recovery of the title compound was 47.5 g 146 mmol, 87% yield. ¹H NMR (400 MHZ, DMSO-d₆) δ ppm 7.63 (t, J=1.7 Hz, 1H), 7.57 (dd, J=9.3, 1.7 Hz, 1H), 7.46-7.32 (m, 5H), 5.36 (s, 2H).

### Example 1B: 2-(benzyloxy)-4-bromo-6-fluoroaniline

To a suspension of the product from Example 1A (5.68 g, 17.4 mmol) and zinc dust (5.70 g, 87 mmol) in a mixture of tetrahydrofuran (56.8 mL) and methanol (56.8 mL) was added saturated aqueous ammonium chloride (28.4 mL) slowly via addition funnel so that the internal temperature remained below 30° C. After stirring vigorously for 1 hour, the mixture was filtered through Celite® (5 g), and the solids were washed with ethyl acetate (56.8 mL). The filtrate was washed with brine (56.8 mL), and then the aqueous layer was extracted with ethyl acetate (28.4 mL). The combined organic layers were washed with water (28.4 mL), then brine (22.7 mL), dried over anhydrous sodium sulfate, filtered and concentrated in vacuo to give the title compound (5.2 g, 17.5 mmol, 100% yield) which was used for the next step without purification. ¹H NMR (400 MHZ, DMSO-d₆) δ ppm 7.52-7.45 (m, 2H), 7.43-7.36 (m, 2H), 7.36-7.30 (m, 1H), 6.99-6.93 (m, 2 h), 5.16 (s, 2H), 4.83 (s, 2H); MS (ESI⁺) m/z 296 [M+H]⁺.

### Example 1C: N-(2-(benzyloxy)-4-bromo-6-fluorophenyl)-2,2,2-trifluoroacetamide

To a solution of the product from Example 1B (5.6 g, 18.96 mmol) and pyridine (2.30 mL, 28.4 mmol) in acetonitrile (56 mL) at an internal temperature below: 16° C. was added trifluoroacetic anhydride (3.48 mL, 24.6 mmol) slowly. After 5 minutes, the reaction mixture was diluted with dichloromethane (56 mL) and water (56 mL). The aqueous layer was extracted with dichloromethane (28 mL), and the combined organic layers were washed with brine (28 mL), dried over anhydrous sodium sulfate, filtered and concentrated in vacuo to give the title compound (7.41 g, 18.9 mmol, 100% yield) which was used for the next step without purification. ¹H NMR (400 MHZ, DMSO-d₆) δ ppm 11.04 (s, 1H), 7.45-7.29 (m, 8H), 5.24 (s, 2H); MS (ESI⁻) m/z 390 [M−H]⁻.

### Example 1D: methyl 2-(N-(2-(benzyloxy)-4-bromo-6-fluorophenyl)-2,2,2-trifluoroacetamido)acetate

To a suspension of the product from Example 1C (7.40 g, 18.9 mmol) and potassium carbonate (7.82 g, 56.6 mmol) in dimethylformamide (37 mL) was added methyl bromoacetate (2.09 mL, 22.6 mmol). The resulting suspension was heated to an internal temperature of 60° C., for 30 minutes, then cooled to room temperature and quenched with 1 M hydrochloric acid (74 mL). The crude aqueous mixture was extracted with ethyl acetate (74 mL, 2×37 mL), and the combined organic layers were washed with saturate aqueous ammonium chloride (2×37 mL), followed by brine (37 mL), dried over sodium sulfate, filtered and concentrated in vacuo to give the crude title compound (9.130 g, 19.67 mmol, 104% yield) which was used for the next step without purification assuming (100% yield). ¹H NMR (400 MHZ, DMSO-d₆) δ ppm 7.47-7.30 (m, 7H), 5.25 (d, J=11.8 Hz, 1H), 5.21 (d, J=11.9 Hz, 1H), 4.52 (d, J=17.0 Hz, 1H), 4.29 (d. J=17.0 Hz, 1H), 3.60 (s, 3H); MS (ESI⁻) m/z 481 [M−H]⁻.

### Example 1E: methyl 2-((2-(benzyloxy)-4-bromo-6-fluorophenyl)amino)acetate

To a solution of the product from Example 1D (8.76 g, 18.87 mmol) in methanol (76.8 mL) was added a solution of sodium methoxide (10.8 mL, 47.2 mmol, 25 weight % in methanol), and the resulting solution was heated to an internal temperature of 60° C. After 10 minutes, the reaction mixture was cooled to room temperature, diluted with ethyl acetate (87.6 mL), quenched with saturated aqueous ammonium chloride (17.5 mL) and diluted with water (43.8 mL). The aqueous layer was extracted with ethyl acetate (2×43.8 mL), and the combined organic layers were washed with brine (26.3 mL), dried over sodium sulfate, filtered and concentrated in vacuo to give the crude title compound (7.281 g, 19.77 mmol, 105% yield) that was used for the next step without purification, assuming 100% yield. ¹H NMR (400 MHz, DMSO-d₆) δ ppm 7.49-7.45 (m, 2H), 7.44-7.38 (m, 2H), 7.37-7.32 (m, 1H), 7.02-7.00 (m, 1H), 6.98 (dd, J=11.8, 2.2 Hz, 1H), 5.22 (td, J=6.9, 2.7 Hz, 1H), 5.16 (s, 2H), 4.04 (dt, J=7.0, 3.8 Hz, 2H), 3.59 (s, 3H); MS (ESI⁺) m/z 368 [M+H]⁺.

### Example 1F: methyl 2-((2-(benzyloxy)-4-bromo-6-fluorophenyl)(N-(tert-butoxycarbonyl)sulfamoyl)amino)acetate

To a solution of chlorosulfonyl isocyanate (2.46 mL, 28.3 mmol) in dichloromethane at 0° C. was added tert-butanol (2.71 mL, 28.3 mmol) slowly so that the internal temperature remained below 10° C. After stirring for 30 minutes, a preformed solution of the product from Example 1E (6.95 g, 18.88 mmol) and triethylamine (5.26 mL, 37.8 mmol) in dichloromethane (27.8 mL) were added dropwise via addition funnel so that the internal temperature did not rise above 10° C. After 30 minutes, the reaction mixture was warmed to room temperature and then quenched with water (70 mL). The layers were separated and the aqueous layer was extracted with dichloromethane (2×35 mL). The combined organic layers were washed with 1 M aqueous sodium bisulfate (40) mL), dried over sodium sulfate, filtered and concentrated in vacuo. The crude solid was precipitated from 1:1 heptanes/ethyl acetate (24 mL), washed with cold heptanes (21 mL) and dried in a vacuum oven at 60° C. to constant weight to give the title compound (9.8188 g, 17.94 mmol, 95% yield). ¹H NMR (400 MHZ, DMSO-d₆) δ ppm 11.31 (s, 1H), 7.48-7.41 (m, 2H), 7.40-7.34 (m, 2H), 7.34-7.26 (m, 1H), 7.22 (dd, J=9.0, 2.1 Hz, 1H), 7.15 (t, J=1.8 Hz, 1H), 5.24 (d, J=13.0 Hz, 1H), 5.18 (d, J=13.0 Hz, 1H), 4.60 (d, J=17.8 Hz, 1H), 4.34 (d, J=17.8 Hz, 1H), 3.52 (s, 3H), 1.28 (s, 9H); MS (ESI⁻) m/z 545 [M−H]⁻.

### Example 1G: methyl 2-((2-(benzyloxy)-4-bromo-6-fluorophenyl)(sulfamoyl)amino)acetate

To a solution of the product from Example 1F (25.1 g, 45.9 mmol) in dichloromethane (100 mL) was added trifluoroacetic acid (53.0 mL, 688 mmol). After 30 minutes, the reaction was diluted with chloroform (125 mL) and concentrated in vacuo. The crude residue was diluted with ethyl acetate (150 mL) and quenched with saturated aqueous disodium phosphate (200 mL) to a final pH of 7. The layers were separated, and the aqueous layer was extracted with ethyl acetate (125 mL). The combined organic layers were washed with brine (75 mL), dried over sodium sulfate, filtered and concentrated to give the title compound (21.76 g, 48.7 mmol, 106% yield) as a thick yellow syrup, which was used for the next step without purification assuming 100% yield. ¹H NMR (500 MHZ, DMSO-d₆) δ ppm 7.53-7.49 (m, 2H), 7.43-7.39 (m, 2H), 7.37-7.32 (m, 1H), 7.24-7.18 (m, 2H), 7.06 (s, 2H), 5.21 (s, 2H), 4.40 (d, J=17.8 Hz, 1H), 4.22 (d, J=17.8 Hz, 1H), 3.57 (s, 3H); MS (ESI⁺) m/z 447 [M+H]⁺.

### Example 1H: 5-[2-(benzyloxy)-4-bromo-6-fluorophenyl]-1λ⁶,2,5-thiadiazolidine-1,1,3-trione

To a solution of the product from Example 1G (29.769 g, 66.6 mmol) in tetrahydrofuran (300 mL) was added a solution of sodium methoxide (22.8 mL, 100 mmol, 25 weight % in methanol) slowly via syringe. After 30 minutes, the reaction was quenched with 1 M hydrochloric acid (150 mL), and extracted with ethyl acetate (3×150 mL). The combined organic layers were washed with brine (90 mL), dried over sodium sulfate, filtered and concentrated. The residue was dissolved in ethyl acetate (180 mL) by heating to 80° C. Heptanes (90 mL) were added dropwise via addition funnel while maintaining the temperature. Upon complete addition, the suspension was slowly cooled to room temperature, and the resulting solid collected by filtration and dried in a vacuum oven at 50° C. to constant weight to give the title compound (17.564 g, 42.3 mmol, 64% yield). ¹H NMR (400 MHZ, DMSO-d₆) δ ppm 7.53-7.46 (m, 2H), 7.40-7.25 (m, 3H), 7.22-7.15 (m, 2H), 7.13 (s, 4H), 5.19 (s, 2H), 3.95 (s, 2H); MS (ESI⁻) m/z 414 [M−H]⁻.

### Example 1I: 5-[6-(benzyloxy)-4-bromo-2-fluoro-3-(prop-2-en-1-yl)phenyl]-1λ⁶,2,5-thiadiazolidine-1,1,3-trione

To a solution of 2,2,6,6-tetramethylpiperidine (0.474 mL, 3.13 mmol) in tetrahydrofuran (5 mL) at 0° C. was added a solution of n-butyllithium (1.2 mL, 3 mmol, 2.5 M in hexane) slowly over 5 minutes. The resulting solution was stirred for 30 minutes, then cooled to an internal temperature of −78° C., and a solution of the product from Example 1H (0.5 g, 1.204 mmol) in tetrahydrofuran (2.5 mL) was slowly added along the side of the flask so that the internal temperature remained below −65° C., followed by N,N,N′,N′-tetramethylethylenediamine (0.200 mL, 1.325 mmol). The resulting red solution was stirred for 1 hour at −78° C., and then allyl bromide (0.11 mL, 1.271 mmol) was added via syringe. The resulting solution was allowed to slowly warm to room temperature overnight, then quenched with 1 M hydrochloric acid, and diluted with ethyl acetate. The layers were separated, and the aqueous layer was extracted with ethyl acetate (2×). The combined organic layers were washed with brine, dried over sodium sulfate, filtered and concentrated in vacuo. The residue was dissolved in dichloromethane, triethylamine (0.336 mL, 2.408 mmol) was added, and the crude material was loaded onto a 40 g Gold® Teledyne ISCO column, then purified by column chromatography with a gradient of 0-10% methanol in dichloromethane (with 0.1% triethylamine added) to give the title compound as a triethylamine salt (0.3915 g, 0.352 mmol, 29.2% yield). ¹H NMR (400 MHZ, DMSO-d₆) ppm 7.53-7.04 (m, 6H), 5.90-5.70 (m, 1H), 5.14 (s, 2H), 5.01 (dt, J=10.1, 1.7 Hz, 1H), 4.95 (dt, J=17.2, 1.9 Hz, 1H), 3.92 (s, 2H), 3.43-3.35 (m, 2H); MS (ESI⁻) m/z 454 [M−H]⁻.

### Example 1J: 5-[6-(benzyloxy)-4-bromo-2-fluoro-3-(3-hydroxypropyl)phenyl]-1λ⁶,2,5-thiadiazolidine-1,1,3-trione

To a solution of the product from Example 1I (0.3910 g, 0.703 mmol, triethylamine salt) in tetrahydrofuran (7.8 mL) was added a solution of 9-borabicyclo[3.3.1]nonane (3.4 mL, 1.7 mmol, 0.5 M in tetrahydrofuran) slowly over 5 minutes. After 2 hours, the reaction mixture was cooled to 0° C., and 1 M aqueous sodium hydroxide (1.7 mL, 1.7 mmol) was added slowly so that the internal temperature remained below 6° C., followed by dropwise addition of aqueous hydrogen peroxide (0.301 mL, 4.92 mmol, 50 weight % in water) so that the internal temperature remained below 15° C. After 1 hour, the reaction mixture was quenched by adding 1 M hydrochloric acid, followed by 1 M aqueous sodium thiosulfate. The crude mixture was extracted with ethyl acetate (3×), and the combined organic layers were washed with brine, dried over sodium sulfate, filtered and concentrated in vacuo. The residue was dissolved in 1:1 dichloromethane/acetonitrile and triethylamine (0.196 mL, 1.405 mmol) was added, then the solution was loaded onto a 40 g Gold® Teledyne ISCO column and was purified by running a gradient of 0-10% methanol in dichloromethane (with 0.1% triethylamine added) to give the title compound as the triethylamine salt (0.2796 g, 0.487 mmol, 69.3% yield). ¹H NMR (501 MHZ, DMSO-d₆) δ ppm 7.51-7.44 (m, 2H), 7.39-7.27 (m, 3H), 7.23-7.14 (m, 1H), 4.54 (t, J=5.1 Hz, 1H), 5.16 (s, 2H), 3.96 (s, 2H), 3.44 (q, J=6.2 Hz, 2H), 2.66 (td, J=8.0, 2.1 Hz, 2H), 1.66-1.56 (m, 2H); MS (ESI⁺) m/z 471 [M−H]⁻.

### Example 1K: 5-[7-(benzyloxy)-5-fluoro-3,4-dihydro-2H-1-benzopyran-6-yl]-1λ⁶,2,5-thiadiazolidine-1,1,3-trione

In a 20 mL pressure release vial, to a mixture of cesium carbonate (0.381 g, 1.170 mmol), 2-di(tert-butyl)phosphino-2′,4′,6′-triisopropyl-3-methoxy-6-methylbiphenyl (RockPhos, 9 mg, 0.019 mmol), and [(2-di-tert-butylphosphino-3-methoxy-6-methyl-2′,4′,6′-triisopropyl-1,1′-biphenyl)-2-(2-aminobiphenyl)]palladium(II) methanesulfonate (RockPhos Pd G3 precatalyst, 16 mg, 0.019 mmol) was added a suspension of the product from Example 1J (0.224 g, 0.390 mmol, triethylamine salt) in N,N-dimethylacetamide (6.5 mL). The resulting suspension was degassed by 5 cycles of vacuum and nitrogen backfills, and then heated to 100° C. After 4 hours the reaction mixture was cooled to room temperature and quenched with 1 M hydrochloric acid. The crude mixture was extracted with ethyl acetate (3×). Then the combined organic layers were washed with saturated aqueous ammonium chloride (3×) and brine. The combined aqueous layers were back extracted with ethyl acetate, and the combined organic layers were dried over sodium sulfate, filtered and concentrated in vacuo to give the crude title compound as an orange oil, which was used for the next step without purification. MS (APCI⁻) m/z 391 [M−H]⁻.

### Example 1L: 5-(5-fluoro-7-hydroxy-3,4-dihydro-2H-1-benzopyran-6-yl)-1λ⁶,2,5-thiadiazolidine-1,1,3-trione

To a suspension of the product of Example 1K (0.191 g, 0.487 mmol) and pentamethylbenzene (0.144 g, 0.973 mmol) in dichloromethane (3.8 mL) at −78° C. was added a solution boron trichloride (1.46 mL, 1.46 mmol, 1 M in dichloromethane) slowly along the side of the flask so that the internal temperature did not rise above −70° C. Upon complete addition, the cooling bath was removed, and the reaction mixture was allowed to warm to 0° C.), then re-cooled to −78° C. and quenched with ethyl acetate (2 mL), followed by ethanol (2 mL) and warmed to room temperature. The crude reaction mixture was concentrated in vacuo to give a residue which was triturated with heptanes (3×5 mL) and 1:1 heptanes/ethyl acetate (2×5 mL). The solid was further purified by reverse phase preparative HPLC on a Phenomenex® Luna® C8(2) 5 μm 100 Å AXIA™ column (30 mm×75 mm) using a gradient of acetonitrile (A) and 10 mM ammonium acetate in water (B), at a flow rate of 50 mL/minute (0-1.0 minute 5% A, 1.0-8.5 minutes linear gradient 5-100% A, 8.5-11.5 minutes 100% A, 11.5-12.0 minutes linear gradient 95-5% A) to give the title compound as the ammonium salt (13.0 mg, 0.041 mmol. 8.4% yield). ¹H NMR (400 MHZ, DMSO-d₆) δ ppm 6.08 (d, J=1.8 Hz, 1H), 4.07 (dd, J=5.9, 4.2 Hz, 2H), 3.89 (s, 2H), 2.55 (t, J=6.4 Hz, 2H), 1.86 (qd, J=6.4, 4.1 Hz, 2H); MS (ESI⁻) m/z 301 [M−H]⁻.

### Example 2: 5-{1-fluoro-3-hydroxy-7-[(4-methoxy-3,3-dimethylbutyl)amino]-5,6,7,8-tetrahydronaphthalen-2-yl}-1λ⁶,2,5-thiadiazolidine-1,1,3-trione (Compound 121)

### Example 2A: 6-bromo-8-fluoro-3,4-dihydronaphthalen-2(1H)-one

To a slurry of 4-bromo-2-fluorophenylacetic acid (10.0 g, 42.9 mmol) in dichloroethane (100 mL) at room temperature was added N,N-dimethylformamide (5 drops) followed by 2 M oxalyl chloride in dichloromethane (23.6 mL, 47.2 mmol). After 90 minutes, the reaction was complete and used directly in the next reaction without concentration or further workup. A small sample was taken for analytical analysis. ¹H NMR (400 MHz, DMSO-d₆) δ ppm 7.51 (td, J=9.4, 2.0 Hz, 1H), 7.40-7.26 (m, 2H), 3.63 (s, 2H).

To a solution of aluminum trichloride (7.44 g, 55.8 mmol) in dichloromethane (200 mL) at −10° C., was added the acid chloride solution from above at such a rate as to maintain the internal temperature below −2° C. Stirring was continued for 15 minutes. To the mixture was introduced a gentle stream of ethylene (internal temperature at −4° C.). After 1 hour, the gas flow was shut off, and the mixture was stirred an additional 10 minutes at −2° C. The reaction was slowly quench with ice water via 2 mL pipet aliquots until the internal temperature stopped rising (approximately 16 to 20 mL water added; internal temperature at 10° C.). Then additional water (500 mL) was added, the ice bath was removed, and the mixture was stirred for 10 minutes to final internal temperature of 20° C. The mixture was transferred to a separatory funnel, and the organic phase washed with brine; then dried (Na₂SO₄), filtered and concentrated to provide 12.6 g of the title compound which was used for the next step without purification. A small sample was taken for analytical analysis. ¹H NMR (500 MHZ, methanol-d₄) δ ppm 7.11 (dd, J=2.0, 1.1 Hz, 1H), 7.06 (dd, J=9.2, 1.9 Hz, 1H), 2.82 (m, 2H), 1.97 (m, 2H).

### Example 2B: 6′-bromo-8′-fluoro-3′,4′-dihydro-1′H-spiro[[1,3]dioxolane-2,2′-naphthalene]

To a solution of the product from Example 2A (10.4 g, 42.9 mmol) and ethylene glycol (14.5 mL, 257 mmol) in toluene (100 mL) at room temperature was added 4-methylbenzenesulfonic acid hydrate (1.63 g, 8.58 mmol); the flask was fitted with a Dean-Stark trap and heated to reflux. After 1 hour, the reaction was cooled to room temperature, transferred to a separatory funnel with ethyl acetate (500 mL) and washed with saturated aqueous sodium bicarbonate (2×300 mL), water (200 mL) and brine (200 mL). The organic fraction was then dried (Na₂SO₄), filtered and concentrated. The residue was purified by chromatography (750 g silica; 1 hour gradient elution from 0% to 20% ethyl acetate:heptanes) to provide 8.74 g (42.9 mmol, 90% pure, 63.8% yield) of the title compound. ¹H NMR (400 MHZ, DMSO-d₆) δ ppm 7.29 (dd, J=9.2, 1.9 Hz, 1H), 7.23 (dd, J=2.0, 1.0 Hz, 1H), 4.00-3.90 (m, 4H), 2.91 (app t, J=6.7 Hz, 2H), 2.76 (s, 2H), 1.85 (app t, J=6.7 Hz, 2H).

### Example 2C 8′-fluoro-3′,4′-dihydro-1′H-spiro[[1,3]dioxolane-2,2′-naphthalen]-6′-ol

To a solution of the product of Example 2B (12.1 g, 42.2 mmol), water (3.8 mL, 210) mmol) and cesium carbonate (28 g, 84 mmol) in N,N-dimethylacetamide (100 mL) at room temperature was added t-BuBrettPhos Pd G3 precatalyst (1.4 g, 1.7 mmol). The reaction was degassed (3× vacuum/purge with nitrogen) followed by heating to 90° C. After 90 minutes, the reaction was cooled to room temperature and transferred to a separatory funnel with water (200 mL) and ethyl acetate (600 mL). To this was added 1 M hydrochloric acid (500 mL) to adjust the aqueous phase to pH to 3. The layers were separated, and the organic phase was washed with water (3×400 mL) and brine (1×400 mL); then dried (Na₂SO₄), filtered and concentrated. Two reaction batches were combined and purified by chromatography (750 g silica; gradient elution 0% to 40% ethyl acetate:0.1% triethylamine in heptanes) to provide 9.34 g (41.8 mmol, 49%) of the title compound. ¹H NMR (400 MHZ, DMSO-d₆) δ ppm 9.52 (s, 1H), 6.36 (d, J=9.2 Hz, 2H), 3.99-3.87 (m, 4H), 2.80 (t, J=6.7 Hz, 2H), 2.68 (s, 2H), 1.80 (t, J=6.7 Hz, 2H); MS (ESI⁻) m/z 223 [M−H]⁻.

### Example 2D: 8′-fluoro-6′-[(2-methoxyethoxy)methoxy]-3′,4′-dihydro-1′H-spiro[[1,3]dioxolane-2,2′-naphthalene]

To a solution of the product from Example 2C (3.6628 g, 16.34 mmol) and 2-methoxyethoxymethyl chloride (2.77 mL, 24.5 mmol) in tetrahydrofuran (72 mL) at room temperature was added N,N-diisopropylethylamine (5.71 mL, 32.7 mmol). The resulting solution was then heated to an internal temperature of 60° C. After 24 hours, the reaction mixture was cooled to room temperature, diluted with ethyl acetate (36 mL) and water (36 mL), and the layers were separated. The aqueous layer was extracted with ethyl acetate (2×25 mL). The combined organic layers were washed with 1 M aqueous sodium bisulfate (36 mL) followed by brine (18 mL), dried over sodium sulfate, filtered and concentrated in vacuo. The residue was loaded onto an 80 g silica gel column in dichloromethane and purified by running a gradient of 0-30% ethyl acetate in heptanes containing 0.1% triethylamine to give the title compound (2.9903 g, 9.57 mmol, 58.6% yield). ¹H NMR (500 MHZ, CDCl₃) δ ppm 6.64 (dd, J=2.5, 1.1 Hz, 1H), 6.61 (dd, J=11.0, 2.4 Hz, 1H), 5.20 (s, 2H), 4.08-3.98 (m, 4H), 3.83-3.76 (m, 2H), 3.59-3.52 (m, 2H), 3.38 (s, 3H), 2.96 (t, J=6.7 Hz, 2H), 2.85 (s, 2H), 1.96-1.89 (m, 2H); MS (APCI⁺) m/z 237 [M-(OCH₂CH₂OCH₃)]⁺.

### Example 2E: 8′-fluoro-7′-iodo-6′-[(2-methoxyethoxy)methoxy]-3′,4′-dihydro-1′H-spiro[[1,3]dioxolane-2,2′-naphthalene]

To a solution of 2,2,6,6-tetramethylpiperidine (4.30 mL, 25.3 mmol) in tetrahydrofuran (100 mL) at 0° C. was added a solution of n-butyllithium (9.49 mL, 23.72 mmol, 2.5 M in hexane) dropwise so that the internal temperature remained below 7° C. After 30 minutes, the solution was cooled to an internal temperature of −74° C., and then a solution of the product of Example 2D (4.94 g, 15.82 mmol) in tetrahydrofuran (25 mL) was added slowly along the side of the flask at a rate so that the internal temperature remained below −70° C., followed by dropwise addition of N,N,N′,N′-tetramethylethylenediamine (3.58 mL, 23.72 mmol). The resulting solution was stirred for 2 hours at −78° C., then a solution of iodine (8.03 g, 31.6 mmol) in tetrahydrofuran (25 mL) was added dropwise so that the internal temperature remained below −65° C. Upon complete addition, the reaction mixture was allowed to warm to 0° C. The resulting suspension was quenched with a 1:1 mixture of saturated aqueous ammonium chloride and 1 M aqueous sodium thiosulfate (50 mL), stirred for 5 minutes at room temperature, and then extracted with ethyl acetate (50 mL, 2× 25 mL). The combined organic layers were washed with water (50 mL), and brine (20 mL), then dried over sodium sulfate, filtered and partially concentrated in vacuo to approximately 50 mL of total volume. Silica (20 g) was added, and the resulting suspension was concentrated in vacuo. The resulting yellow powder was dry loaded onto a 120 g silica gel column, and eluted with a gradient of 0-30% ethyl acetate in heptanes containing 0.1% triethylamine to give the title compound (5.6776 g, 12.96 mmol, 82% yield). ¹H NMR (400 MHZ, CDCl₃) δ ppm 6.73 (d, J=1.4 Hz, 1H), 5.30 (s, 2H), 4.09-3.97 (m, 4H), 3.88-3.76 (m, 2H), 3.60-3.52 (m, 2H), 3.38 (s, 3H), 2.96 (t, J=6.7 Hz, 2H), 2.88 (s, 2H), 1.92 (t, J=6.7 Hz, 2H); MS (APCI+) m/z 363 [M-(OCH₂CH₂OCH₃)]⁺.

### Example 2F: tert-butyl ({8′-fluoro-6′-[(2-methoxyethoxy)methoxy]-3′,4′-dihydro-1′H-spiro[[1,3]dioxolane-2,2′-naphthalen]-7′-yl}amino)acetate

In a 500 mL round-bottom flask were combined cesium carbonate (7.70 g, 23.63 mmol), BrettPhos (0.127 g, 0.236 mmol), BrettPhos Pd G3 precatalyst (0.214 g, 0.236 mmol) and the product from Example 2E (5.1776 g, 11.81 mmol). The flask was placed under vacuum for 5 minutes, and refilled with nitrogen. 1,4-Dioxane (104 mL) was added followed by tert-butyl 2-aminoacetate (1.94 mL, 14.18 mmol). The resulting suspension was degassed by 5× vacuum/nitrogen backfills, stirred for 5 minutes at room temperature, and then heated to an internal temperature of 90° C. After 2 hours, the mixture was cooled to below 40° C. and another portion of BrettPhos (0.127 g, 0.236 mmol) and BrettPhos Pd G3 precatalyst (0.214 g, 0.236 mmol) were added. The resultant mixture was degassed by 3× vacuum/nitrogen backfills and then heating to 90° C. was resumed. After 90 minutes, the reaction mixture was cooled to below 40° C. and another portion of BrettPhos (0.127 g, 0.236 mmol) and BrettPhos Pd G3 precatalyst (0.214 g, 0.236 mmol) were added. The mixture was degassed by 3× vacuum/nitrogen backfills and heating to 90° C. was again resumed. After 24 hours, the reaction mixture was cooled to room temperature and quenched with saturated aqueous ammonium chloride (15 mL), diluted with water (35 mL), and extracted with ethyl acetate (50 mL, 2× 25 mL). The combined organic layers were washed with brine (20 mL), dried over sodium sulfate, filtered and concentrated in vacuo. The residue was loaded onto an 80 g silica gel column in dichloromethane and eluted with a gradient of 0-50% ethyl acetate in heptanes containing 0.1% triethylamine to give the title compound (4.4284 g, 10.03 mmol, 85% yield). ¹H NMR (400 MHz, CDCl₃) δ ppm 6.80-6.55 (m, 1H), 5.25 (s, 2H), 4.36 (td, J=6.0, 2.8 Hz, 1H), 4.09-3.97 (m, 4H), 3.93 (dd, J=6.1, 2.0 Hz, 2H), 3.86-3.80 (m, 2H), 3.62-3.51 (m, 2H), 3.39 (s, 3H), 2.88 (t, J=6.7 Hz, 2H), 2.83 (s, 2H), 1.89 (t, J=6.7 Hz, 2H), 1.45 (s, 9H); MS (ESI⁺) m/z 442 [M+H]⁺.

### Example 2G; tert-butyl [{8′-fluoro-6′-[(2-methoxyethoxy)methoxy]-3′,4′-dihydro-1′H-spiro[[1,3]dioxolane-2,2′-naphthalen]-7′-yl}({[(prop-2-en-1-yl)oxy]carbonyl}sulfamoyl)amino]acetate

To a solution of chlorosulfonyl isocyanate (1.42 mL, 16.29 mmol) in dichloromethane (48 mL) at 0° C. was added allyl alcohol (1.11 mL, 16.29 mmol) dropwise so that the internal temperature remained below 10° C. After 30 minutes, a preformed solution of the product of Example 2F (4.7953 g, 10.86 mmol) and N,N-diisopropylethylamine (3.79 mL, 21.72 mmol) in dichloromethane (24 mL) was added slowly so that the internal temperature remained below 10° C. After 30 minutes, the reaction mixture was quenched with water (48 mL), stirred for 5 minutes, and then the layers were separated. The aqueous layer was extracted with dichloromethane (2×24 mL). The combined organic layers were washed with 1 M aqueous sodium bisulfate (24 mL), and then the new aqueous layer was back extracted with dichloromethane (15 mL). The combined organic layers were dried over sodium sulfate, filtered and concentrated in vacuo to give the title compound, which was used for the next step without purification. ¹H NMR (400 MHZ, DMSO-d₆) δ ppm 11.46 (s, 1H), 6.81 (s, 1H), 5.91 (ddt, J=17.2, 10.6, 5.3 Hz, 1H), 5.37-5.28 (m, 1H), 5.25 (d, J=7.1 Hz, 1H), 5.22 (d, J=9.8 Hz, 1H), 5.14 (d, J=6.8 Hz, 1H), 4.70 (d, J=17.5 Hz, 1H), 4.63-4.48 (m, 4H), 4.08 (d, J=17.6 Hz, 1H), 4.01-3.88 (m, 4H), 3.72 (qt, J=11.2, 4.7 Hz, 2H), 3.46 (t, J=4.7 Hz, 2H), 3.23 (s, 3H), 2.87 (t, J=6.7 Hz, 2H), 2.71 (s, 2H), 1.84 (t, J=6.6 Hz, 2H), 1.33 (s, 9H); MS (ESI⁺) m/z 622 [M+NH₄]⁺.

### Example 2H: 5-{8′-fluoro-6′-[(2-methoxyethoxy)methoxy]-3′,4′-dihydro-1′H-spiro[[1,3]dioxolane-2,2′-naphthalen]-7′-yl}-1λ⁶,2,5-thiadiazolidine-1,1,3-trione

To a solution of the product of Example 2G (6.57 g, 10.87 mmol) in methanol (117 mL) was added tetrakis(triphenylphosphine)palladium(0)) (0.251 g, 0.217 mmol). The resulting suspension was degassed by 5× vacuum/nitrogen backfills, then a solution of sodium methoxide (14.9 mL, 65.2 mmol, 25 w % in methanol) was added and the resulting suspension was heated to an internal temperature of 60° C. After 1 hour, the mixture was cooled to room temperature, diluted with ethyl acetate (66 mL), and partially concentrated to approximately 33 mL total volume to remove methanol. The resulting suspension was diluted with ethyl acetate (66 mL) and quenched with 1 M hydrochloric acid (70 mL, final pH<3). The aqueous layer was extracted with ethyl acetate (2×33 mL). The combined organic layers were washed with brine (19 mL), dried over sodium sulfate, filtered through Celite® (5 g disposable frit) and concentrated in vacuo. The residue was chased with acetonitrile (33 mL) and concentrated to give the title compound (4.6781 g, 10.48 mmol, 96% yield), which was used for the next step without further purification. ¹H NMR (400 MHZ, DMSO-d₆) δ ppm 6.83 (d, J=1.5 Hz, 1H), 5.25 (s, 2H), 4.35 (s, 2H), 3.99-3.86 (m, 4H), 3.81-3.69 (m, 2H), 3.50-3.39 (m, 2H), 3.23 (s, 3H), 2.89 (t, J=6.7 Hz, 2H), 2.74 (s, 2H), 1.85 (t, J=6.6 Hz, 2H); MS (ESI⁻) m/z 445 [M−H]⁻.

### Example 2I: 5-{1-fluoro-3-[(2-methoxyethoxy)methoxy]-7-oxo-5,6,7,8-tetrahydronaphthalen-2-yl}-1λ⁶,2,5-thiadiazolidine-1,1,3-trione, triethylamine salt

The product of Example 2H (2.6869 g, 6.02 mmol) was suspended in formic acid (13.4 mL, 307 mmol, 88%), quickly becoming a yellow suspension. After 15 minutes, the reaction mixture was diluted with a slow addition of brine (54 mL). The aqueous mixture was extracted with a 2:1 mixture of ethyl acetate and acetonitrile (3×27 mL). The combined organic layers were washed with brine (2×13 mL), dried over sodium sulfate, and filtered. To the crude solution was added triethylamine (2.52 mL, 18.06 mmol) and silica (10 g), and the resulting suspension was concentrated in vacuo. The resulting yellow powder was dry loaded onto an 80 g silica gel column and eluted with a gradient of 0-20% methanol in dichloromethane containing 0.2% triethylamine to give the title compound (3.2400 g, 6.02 mmol, 100% yield) as a hygroscopic yellow solid. ¹H NMR (400 MHZ, DMSO-d₆) δ ppm 6.93 (d, J=1.5 Hz, 1H), 5.24 (s, 2H), 3.94 (s, 1H), 3.80-3.71 (m, 2H), 3.50-3.41 (m, 3H), 3.23 (s, 2H), 3.17 (s, 3H), 3.02 (dd, J=7.6, 5.9 Hz, 2H), 2.56-2.40 (m, 2H); MS (ESI⁺) m/z 420 [M+NH₄]⁺.

### Example 2J: 5-{1-fluoro-3-hydroxy-7-[(4-methoxy-3,3-dimethylbutyl)amino]-5,6,7,8-tetrahydronaphthalen-2-yl}-1λ⁶,2,5-thiadiazolidine-1,1,3-trione

To a solution of the product from Example 21 (0.100 g, 0.186 mmol) and 4-methoxy-3,3-dimethylbutan-1-amine (0.037 g, 0.279 mmol) in acetonitrile (2 mL) at room temperature was added sodium cyanoborohydride (0.014 g, 0.223 mmol). After 3 hours, a solution of HCl (0.464 mL, 1.857 mmol, 4 M in dioxane) was added dropwise (vigorous gas evolution). After 90) minutes, the reaction mixture was diluted with acetonitrile (3 mL) and water (1 mL). Celite® (1 g) was added, and the mixture was concentrated in vacuo. The resultant mixture was dry loaded onto a Teledyne ISCO 100 g reverse-phase C18 column, eluted with a gradient of 5-100% methanol in buffer (0.025 M ammonium bicarbonate in water acidified to pH 7 by adding dry ice) to give the title compound (0.0221 g, 0.051 mmol, 27.7% yield). ¹H NMR (400 MHZ, DMSO-d₆) δ ppm 9.21 (br s, 1H), 8.41 (br s, 2H), 6.47 (d, J=1.5 Hz, 1H), 3.93 (s, 2H), 3.49-3.39 (m, 2H), 3.27 (s, 3H), 3.15-3.07 (m, 1H), 3.06 (s, 2H), 3.04-2.96 (m, 2H), 2.87-2.64 (m, 2H), 2.20-2.13 (m, 1H), 1.68 (dq, J=11.2, 5.7 Hz, 1H), 1.62-1.53 (m, 2H), 0.90 (s, 6H); MS (ESI⁺) m/z 430 [M+H]⁺.

### Example 3: 5-(8-fluoro-6-hydroxy-1,2,3,4-tetrahydroisoquinolin-7-yl)-1λ⁶,2,5-thiadiazolidine-1,1,3-trione (Compound 143)

### Example 3A; N-[6-(benzyloxy)-4-bromo-2-fluoro-3-formylphenyl]-2,2,2-trifluoroacetamide

A solution of diisopropylamine (4.80 mL, 33.7 mmol) in tetrahydrofuran (21 mL) was cooled to an internal temperature of −73° C., and n-butyllithium (14.0 mL, 33.7 mmol, 2.5 M in hexanes) was added over 10 minutes. The mixture was stirred for 5 minutes, warmed to 0° C. stirred for 10 minutes, and then re-cooled to −73° C. A solution of N-(2-(benzyloxy)-4-bromo-6-fluorophenyl)-2,2,2-trifluoroacetamide (the compound from Example 1C) (6.00 g, 15.3 mmol) in tetrahydrofuran (41 mL) was cooled to an internal temperature of −76° C., and the lithium diisopropylamide solution prepared above was added at a rate such that the internal temperature did not exceed −70° C. After aging for 75 minutes, a solution of N,N-dimethylformamide (4.74 mL, 185 mmol) in tetrahydrofuran (15 mL) was added at a rate such that the internal temperature did not exceed −68° C. After 20 minutes, the reaction was quenched with saturated aqueous ammonium chloride (30) mL), warmed to room temperature, and partitioned between ethyl acetate (2×50) mL) and water (50 mL). The combined organic extracts were washed with saturated aqueous ammonium chloride (4×20 mL), dried over sodium sulfate, then filtered and concentrated under reduced pressure to afford a viscous oil that was immediately purified by flash chromatography on silica gel [80 g SiO₂, gradient from heptanes→30% ethyl acetate/heptanes, 60 mL/minute] to afford the title compound (3.58 g, 8.42 mmol, 55.6% yield). ¹H NMR (400 MHZ, DMSO-d₆) δ ppm 11.21 (br s, 1H), 10.10 (d, J=1.1 Hz, 1H), 7.55 (d, J=1.4 Hz, 1H), 7.48-7.27 (m, 5H), 5.38 (s, 2H); MS (APCI+) m/z 421 [M+H]⁺.

### Example 3B: tert-butyl {[4-(benzyloxy)-6-bromo-2-fluoro-3-(2,2,2-trifluoroacetamido)phenyl]methyl}{2-[methoxy(methyl)amino]-2-oxoethyl}carbamate

To a solution of 2-amino-N-methoxy-N-methylacetamide hydrobromide (5.86 g, 29.5 mmol) in methanol (89 mL) was added triethylamine (4.11 mL, 29.5 mmol). After 5 minutes, acetic acid (0.766 mL, 13.39 mmol) was added followed by a solution of N-[6-(benzyloxy)-4-bromo-2-fluoro-3-formylphenyl]-2,2,2-trifluoroacetamide (11.25 g, 26.8 mmol) in methanol (89 mL). After 20 minutes, sodium triacetoxy borohydride (11.35 g, 53.6 mmol) was added in one portion, and the reaction mixture was stirred at room temperature for 2 hours. Thereafter, a solution of 2-amino-N-methoxy-N-methylacetamide hydrobromide (1.40 g, 7.03 mmol) and triethylamine (1.20 mL, 8.61 mmol) in methanol (15 mL) was added followed by sodium triacetoxy borohydride (3.00 g, 14.2 mmol). After 25 minutes, the reaction was poured into water (200 mL) and extracted into ethyl acetate (2×100 mL). The combined organic extracts were dried over sodium sulfate, filtered and concentrated under reduced pressure to give 15.48 g of a viscous residue. This was dissolved in dichloromethane (179 mL) and triethylamine (4.11 mL, 29.5 mmol) was added followed by di-tert-butyl dicarbonate (6.43 g, 29.5 mmol), and the reaction mixture was stirred at room temperature. After 14 hours, water (100 mL) was added, and the mixture was extracted into ethyl acetate (2×50 mL). The combined organic layers were dried over sodium sulfate, then filtered and concentrated under reduced pressure to afford 18.7 g of a viscous oil that was purified by flash chromatography on silica gel [220 g SiO₂, heptanes→50% ethyl acetate/heptanes, 150 mL/minute] to afford the title compound (12.1 g, 19.4 mmol, 72.4% yield). MS (APCI⁺) m/z 624 [M+H]⁺.

### Example 3C: tert-butyl 6-(benzyloxy)-8-fluoro-4-oxo-7-(2,2,2-trifluoroacetamido)-3,4-dihydroisoquinoline-2(1H)-carboxylate

A solution of tert-butyl {[4-(benzyloxy)-6-bromo-2-fluoro-3-(2,2,2-trifluoroacetamido)phenyl]methyl}{2-[methoxy(methyl)amino]-2-oxoethyl}carbamate (11.0 g, 17.7 mmol) in tetrahydrofuran (142 mL) was cooled to an internal temperature of −75° C., and n-butyllithium (15.1 mL, 36.3 mmol, 2.5 M in hexanes) was added at a rate such that the internal temperature did not exceed −70° C. After 5 minutes, the reaction was quenched with saturated aqueous ammonium chloride (20 mL), warmed to room temperature, and partitioned between ethyl acetate (150 mL) and water (100 mL). The aqueous layer was back-extracted with ethyl acetate (1×50 mL), and the combined organic extracts were dried over sodium sulfate, then filtered and concentrated under reduced pressure to afford 10.2 g of a viscous oil that was purified by flash chromatography on silica gel [120 g SiO₂, heptanes→30% ethyl acetate/heptanes, 85 mL/minute] to afford the title compound (6.44 g, 13.4 mmol, 68.6% yield). ¹H NMR (400 MHZ, CDCl₃) δ ppm 7.80 (s, 1H), 7.53 (d, J=1.5 Hz, 1H), 7.49-7.31 (m, 5H), 5.19 (s, 2H), 4.78 (s, 2H), 4.33 (s, 2H), 1.49 (s, 9H); MS (ESI⁺) m/z 481 [M−H]⁻.

### Example 3D: tert-butyl 6-(benzyloxy)-8-fluoro-7-[(2-methoxy-2-oxoethyl)(trifluoroacetyl)amino]-4-oxo-3,4-dihydroisoquinoline-2(1H)-carboxylate

To a solution of tert-butyl 6-(benzyloxy)-8-fluoro-4-oxo-7-(2,2,2-trifluoroacetamido)-3,4-dihydroisoquinoline-2(1H)-carboxylate (1.50 g, 3.11 mmol) in anhydrous N,N-dimethylformamide (7.8 mL) was added 1,2,2,6,6-pentamethylpiperidine (1.13 mL, 6.22 mmol) and methyl bromoacetate (0.372 mL, 4.04 mmol), and the reaction was heated to an internal temperature of 60° C. After 1 hour, the mixture was cooled to room temperature and partitioned between ethyl acetate (25 mL) and saturated aqueous ammonium chloride (20 mL). The organic layer was further washed with saturated aqueous ammonium chloride (4×20 mL), dried over sodium sulfate, then filtered and concentrated under reduced pressure to give 2.11 g of an orange oil that was purified by flash chromatography on silica gel [24 g SiO₂, heptanes→25% ethyl acetate/heptanes, 35 mL/minute] to afford the title compound (1.29 g, 2.33 mmol, 74.9% yield). MS (APCI⁺) m/z 574 [M+NH₄]⁺.

### Example 3E: tert-butyl 6-(benzyloxy)-8-fluoro-4-[(1H-imidazole-1-carbothioyl)oxy]-7-[(2-methoxy-2-oxoethyl)(trifluoroacetyl)amino]-3,4-dihydroisoquinoline-2(1H)-carboxylate

To a solution of tert-butyl 6-(benzyloxy)-8-fluoro-7-[(2-methoxy-2-oxoethyl)(trifluoroacetyl)amino]-4-oxo-3,4-dihydroisoquinoline-2(1H)-carboxylate (1.29 g, 2.33 mmol) in anhydrous tetrahydrofuran (23 mL) was added sodium borohydride (0.088 g, 2.33 mmol) in one portion. After 5 minutes, the reaction was diluted with ethyl acetate (20 mL) and quenched with saturated aqueous ammonium chloride (2 mL). The aqueous layer was back-extracted with ethyl acetate (1×20 mL), and the combined organic layers were dried over sodium sulfate, then filtered and concentrated under reduced pressure to give 1.26 g of an oil. The oil was dissolved in dichloromethane (23 mL) and 4-dimethylaminopyridine (0.085 g, 0.698 mmol) was added followed by 1,1′-thiocarbonyldiimidazole (0.539 g, 3.02 mmol). After 45 minutes, the reaction was directly concentrated to give an oil that was immediately purified by flash chromatography on silica gel [24 g SiO₂, heptanes→25% acetone/heptanes, 35 mL/minute, detect at 216 nm] to afford the title compound (1.03 g, 1.54 mmol, 66.3% yield over two steps). MS (APCI⁺) m/z 667 [M+H]⁺.

### Example 3F: tert-butyl 6-(benzyloxy)-8-fluoro-7-[(2-methoxy-2-oxoethyl)(trifluoroacetyl)amino]-3,4-dihydroisoquinoline-2(1H)-carboxylate

To a solution of tert-butyl 6-(benzyloxy)-8-fluoro-4-[(1H-imidazole-1-carbothioyl)oxy]-7-[(2-methoxy-2-oxoethyl)(trifluoroacetyl)amino]-3,4-dihydroisoquinoline-2(1H)-carboxylate (1.029 g, 1.54 mmol) and benzene (26 mL) was added tributyltin hydride (0.457 mL, 1.70 mmol). To the mixture was added a solution of triethylborane (1.70 mL, 1.70 mmol, 1.0 M in tetrahydrofuran) in one portion, and the reaction was stirred at room temperature. After 8 minutes, the reaction was concentrated to 1.5 mL and directly purified by flash chromatography on silica gel [24 g SiO₂, heptanes→20% acetone/heptanes, 35 mL/minute, detect at 208 nm] to afford the title compound (0.705 g, 1.30 mmol, 85% yield). MS (ESI⁻) m/z 539 [M−H]⁻.

### Example 3G; tert-butyl 6-(benzyloxy)-8-fluoro-7-[(2-methoxy-2-oxoethyl)amino]-3,4-dihydroisoquinoline-2(1H)-carboxylate

To a solution of tert-butyl 6-(benzyloxy)-8-fluoro-7-[(2-methoxy-2-oxoethyl)(trifluoroacetyl)amino]-3,4-dihydroisoquinoline-2(1H)-carboxylate (0.660 g, 1.22 mmol) in anhydrous methanol (8.1 mL) was added sodium methoxide (0.70 mL, 3.05 mmol, 25% w/w in methanol), and the reaction was heated to an internal temperature of 50° C. After 2 hours, the reaction was cooled to room temperature and quenched with saturated aqueous ammonium chloride (10 mL). The mixture was partitioned between ethyl acetate (30) mL) and water (10 mL), the aqueous layer was back-extracted with ethyl acetate (2×5 mL), and the combined organic extracts were dried over sodium sulfate, then filtered and concentrated under reduced pressure. To remove adventitious water, the residue was dissolved in ethyl acetate (20 mL), washed with brine (1×10 mL), dried over sodium sulfate, then filtered and concentrated under reduced pressure to afford 0.702 g of an oil that was purified by flash chromatography on silica gel [12 g SiO₂, heptanes→25% acetone/heptanes, 30 mL/minute, detect at 208 nm] to afford the title compound (0.412 g, 0.927 mmol, 71.1% yield). MS (ESI⁺) m/z 445 [M+H]⁺.

### Example 3H: tert-butyl 6-(benzyloxy)-8-fluoro-7-(1,1,4-trioxo-1λ⁶,2,5-thiadiazolidin-2-yl)-3,4-dihydroisoquinoline-2(1H)-carboxylate

To a solution of chlorosulfonyl isocyanate (0.121 mL, 1.39 mmol) in dichloromethane (4.6 mL) at an internal temperature of 0° C. was added allyl alcohol (0.095 mL, 1.39 mmol) at a rate such that the internal temperature did not exceed 7° C. After 30 minutes, a preformed solution of tert-butyl 6-(benzyloxy)-8-fluoro-7-[(2-methoxy-2-oxoethyl)amino]-3,4-dihydroisoquinoline-2(1H)-carboxylate (0.425 g, 0.927 mmol) and N,N-diisopropylethylamine (0.324 mL, 1.854 mmol) in dichloromethane (4.6 mL) was added at a rate such that the internal temperature did not exceed 7° C. After 30 minutes, the reaction was quenched with water (48 mL) and stirred for 5 minutes. Then the layers were separated, and the aqueous layer was extracted with dichloromethane (2×24 mL). The combined organic extracts were dried over sodium sulfate, filtered and concentrated to give 0.530 g of a foam, which was used without purification in the next step.

A solution of the above alloc-sulfonylurea (0.473 g, 0.778 mmol) in anhydrous methanol (8.6 mL) was degassed via sub-surface nitrogen sparging for 15 minutes. Thereafter, tetrakis(triphenylphosphine)palladium(0) (0.018 g, 0.016 mmol) was added followed by a solution of sodium methoxide (1.07 mL, 4.67 mmol, 25% w/w in methanol), and the reaction mixture was heated to a mantle temperature of 60° C. After 15 minutes, the mixture was cooled to room temperature, quenched with 1 M HCl (1 mL), and partitioned between ethyl acetate (4 mL) and water (3 mL). The aqueous layer was extracted with ethyl acetate (2×1 mL), and the combined organic extracts were washed with brine (1×5 mL), dried over sodium sulfate, filtered, and concentrated to afford the title compound (314 mg, 0.639 mmol, 82% yield). MS (ESI⁻) m/z 490 [M−H]⁻.

### Example 3I: 5-(8-fluoro-6-hydroxy-1,2,3,4-tetrahydroisoquinolin-7-yl)-1λ⁶,2,5-thiadiazolidine-1,1,3-trione

A suspension of tert-butyl 6-(benzyloxy)-8-fluoro-7-(1,1,4-trioxo-1λ⁶,2,5-thiadiazolidin-2-yl)-3,4-dihydroisoquinoline-2(1H)-carboxylate (37.7 mg, 0.077 mmol) and 1,2,3,4,5-pentamethylbenzene (34.1 mg, 0.230 mmol) in dichloromethane (0.76 mL) was cooled to −78° C., and a solution of boron trichloride (153 μL, 0.153 mmol, 1.0 M in dichloromethane) was added dropwise over 5 minutes. After 15 minutes, the reaction was quenched with anhydrous methanol (31.0 μL, 0.767 mmol) and warmed to room temperature under nitrogen. The volatiles were removed to afford a solid that was triturated with heptane (3×1 mL) and dichloromethane (2×1 mL). The crude material was thereafter dissolved in water (2 mL), filtered through a plug of cotton to remove a yellow residue, and purified by reverse-phase HPLC [Luna® 10 μm C18(2) 100 Å, AX (00G-4253-U0-AX) column, 250×30 mm, 50 mL/minute, 1 injection, 5%→95% CH₃CN/H₂O (with pure, unbuffered water) over 15 minutes, monitored/collected at 205 nm]. The product eluted with the solvent front and was thereafter lyophilized (0.031 mbar) for 8 hours to afford the title compound (9.3 mg, 0.031 mmol, 40.2% yield). ¹H NMR (400 MHZ, DMSO-d₆) δ ppm 10.49 (br s, 1H), 9.34 (br s, 2H), 6.64 (s, 1H), 4.33 (s, 2H), 4.14 (app t, J=3.8 Hz, 2H); 3.32 (app q, J=5.7 Hz, 2H), 2.94 (t, J=5.9 Hz, 2H); MS (ESI⁻) m/z 300 [M−H]⁻.

### Example 4: 5-[8-fluoro-6-hydroxy-2-(5,5,5-trifluoropentyl)-1,2,3,4-tetrahydroisoquinolin-7-yl]-1λ⁶,2,5-thiadiazolidine-1,1,3-trione (Compound 153)

### Example 4A: 5-[6-(benzyloxy)-8-fluoro-1,2,3,4-tetrahydroisoquinolin-7-yl]-1λ⁶,2,5-thiadiazolidine-1,1,3-trione, trifluoroacetate

Trifluoroacetic acid (0.1 mL, 1.34 mmol, 15.0 equivalents) was added to a suspension of tert-butyl 6-(benzyloxy)-8-fluoro-7-(1,1,4-trioxo-1λ⁶,2,5-thiadiazolidin-2-yl)-3,4-dihydroisoquinoline-2(1H)-carboxylate, the product of Example 3H (44 mg, 0.09 mmol, 1 equivalent), in dichloromethane (0.45 mL) at 23° C. The reaction mixture was stirred for 30 minutes at 23° C. The product mixture was then diluted with ether (1.0 mL) at 23° C. A precipitate immediately formed. The diluted mixture was concentrated under a stream of nitrogen. The titled compound obtained was used without further purification. MS (APCI⁺) m/z 433 [M+H+CH₃CN]⁺.

### Example 4B: 5-[6-(benzyloxy)-8-fluoro-2-(5,5,5-trifluoropentyl)-1,2,3,4-tetrahydroisoquinolin-7-yl]-1λ⁶,2,5-thiadiazolidine-1,1,3-trione

A suspension of 5-[6-(benzyloxy)-8-fluoro-1,2,3,4-tetrahydroisoquinolin-7-yl]-1λ⁶,2,5-thiadiazolidine-1,1,3-trione, trifluoroacetate, the product of Example 4A (nominally 0.09 mmol, 1 equivalent), potassium carbonate (62 mg, 0.45 mmol, 5.0 equivalents), and 5,5,5-trifluoropentyl 4-methylbenzenesulfonate (40 mg, 0.14 mmol, 1.5 equivalents: Erdeljac, N., et al. Chem. Commun, 2018, 54, 12002-12005) in acetonitrile (0.45 mL) was heated to 60° C. with stirring for 19 hours. The reaction mixture was then cooled to 23° C. The cooled reaction mixture was diluted sequentially with aqueous hydrogen chloride solution (1.0 M, 0.5 mL), water (0.5 mL), and dimethyl sulfoxide (1.0 mL). The diluted mixture was purified by reversed-phase flash column chromatography (100 g RediSep Rf Gold® C18 column, elution with a gradient of 10-100% [v/v] methanol-0.025 M aqueous ammonium bicarbonate solution [acidified with solid carbon dioxide] over 10 column volumes, then isocratic elution with 100% methanol for 3 column volumes, flow rate=60 mL/minute). The title compound obtained (44 mg) was used in the following step without further purification. MS (APCI⁺) m/s 516 [M+H]⁺.

### Example 4C: 5-[8-fluoro-6-hydroxy-2-(5,5,5-trifluoropentyl)-1,2,3,4-tetrahydroisoquinolin-7-yl]-1λ⁶,2,5-thiadiazolidine-1,1,3-trione

A solution of boron trichloride in dichloromethane (1.0 M, 0.9 mL, 0.90 mmol, 11.3 equivalents) was added to a suspension of the product of Example 4B (nominally 44 mg, 0.08 mmol, 1 equivalent) and pentamethylbenzene (37 mg, 0.25 mmol, 3.0 equivalents) in dichloromethane (0.85 mL) at −78° C. The reaction mixture was stirred for 4 hours at −78° C. The reaction mixture was then diluted with methanol (0.5 mL) at −78° C. The diluted mixture was warmed over 15 minutes to 23° C. The warmed mixture was concentrated. The residue obtained was purified by reversed-phase flash column chromatography (100 g RediSep Rf Gold® C18 column, elution with a gradient from 10-100% [v/v] methanol-0.025 M aqueous ammonium bicarbonate solution [acidified with solid carbon dioxide] over 10 column volumes, then isocratic elution with 100% methanol for 3 column volumes, flow rate=60 mL/minute) to furnish the title compound (14 mg, 41% yield, two steps). ¹H NMR (400 MHZ, DMSO-d₆) δ ppm 6.52 (s, 1H), 4.10 (q, J=5.2 Hz, 1H), 3.94 (2, 2H), 3.17 (d, J=5.1 Hz, 2H), 2.86 (app bs, 2H), 2.35-2.23 (m, 2H), 1.71 (app bs, 2H), 1.59-1.47; MS (APCI⁺) m/z 426 [M+H]⁺.

### Example 5: 5-{2-[(azetidin-3-yl)methyl]-8-fluoro-6-hydroxy-1,2,3,4-tetrahydroisoquinolin-7-yl}-1λ⁶,2,5-thiadiazolidine-1,1,3-trione (Compound 166)

### Example 5A: 6′-(benzyloxy)-8′-fluoro-3′,4′-dihydro-1′H-spiro[[1,3]dioxolane-2,2′-naphthalene]

To a solution of the product from Example 2B (100 g, 348 mmol) and benzyl alcohol (50.5 mL, 488 mmol) in dioxane (200 mL) was added sodium tert-butoxide (40.2 g, 418 mmol), N,N′-diphenethyloxalamide (1.032 g, 3.48 mmol) and copper (I) iodide (0.663 g, 3.48 mmol). The resulting mixture was degassed (3× vacuum/purge with nitrogen) and then heated to 80° C. After 48 hours, water (1 L) was added, and the resulting mixture was cooled to ambient temperature. The mixture was filtered, and the solid was washed with water (200 mL). The filtrate was extracted with ethyl acetate (3×500 mL). The organic layers were combined, dried over anhydrous sodium sulfate, filtered and concentrated in vacuo. The residue was dissolved in dichloromethane (1 L) and filtered through Celite® (100 g). The filtrate was concentrated in vacuo. The resulting solid was triturated with isopropanol (200 mL) to give 85 g (244 mmol, 78% yield) of the title compound. ¹H NMR (400 MHZ, CDCl₃) δ ppm 7.46-7.28 (m, 5H), 6.74-6.60 (m, 2H), 5.07 (s, 2H), 4.00-3.88 (m, 4H), 2.86 (t, J=6.7 Hz, 2H), 2.72 (s, 2H), 1.83 (t, J=6.7 Hz, 2H); MS (APCI+) m/z 315 [M+H]⁺.

### Example 5B: 6′-(benzyloxy)-7′-bromo-8′-fluoro-3′,4′-dihydro-1′H-spiro[[1,3]dioxolane-2,2′-naphthalene]

To a solution of 2,2,6,6-tetramethylpiperidine (164 mL, 964 mmol) in tetrahydrofuran (500 mL) at 0° C.) was added a solution of n-butyllithium (360 mL, 2.5 M in hexane, 900 mL) slowly over 40 minutes. After stirring for 30 minutes, the reaction mixture was diluted with tetrahydrofuran (500 mL) and cooled to −78° C. A solution of the product of Example 5A (202.11 g, 643 mmol) in tetrahydrofuran (500 mL) was added slowly over 30 minutes so that the internal temperature remained below −70° C. After 2 hours, 1,2-dibromo-1,1,2,2-tetrafluoroethane (92 mL, 772 mmol) was added slowly so that the internal temperature remained below −60° C. Upon complete addition the reaction mixture was warmed to −10° C., then was quenched with saturated aqueous ammonium chloride (500 mL) and diluted with water (1.5 L) and ethyl acetate (2 L). The layers were separated, and the organic layer was washed with 1 M hydrochloric acid, saturated aqueous sodium bicarbonate, and brine (500 mL), then dried over anhydrous sodium sulfate, filtered and concentrated in vacuo. The resulting residue was diluted with isopropanol (500 mL), and then heated to 50° C. and slowly cooled to ambient temperature. The resulting solid was collected by filtration to give the title compound (130.3 g, 331 mmol, 51.5% yield). ¹H NMR (400 MHZ, CDCl₃) δ ppm 7.50-7.28 (m, 5H), 6.53 (d, J=1.6 Hz, 1H), 5.12 (s, 2H), 4.10-3.97 (m, 4H), 2.93 (t, J=6.7 Hz, 2H), 2.89 (s, 2H), 1.92 (t, J=6.7 Hz, 2H); MS (APCI+) m/z 393 [M+H]⁺.

### Example 5C: tert-butyl {[6′-(benzyloxy)-8′-fluoro-3′,4′-dihydro-1′H-spiro[[1,3]dioxolane-2,2′-naphthalen]-7′-yl]amino}acetate

To a suspension of the product from Example 5B (14.17 g, 36 mmol), cesium carbonate (35.2 g, 108 mmol), BrettPhos (0.387 g, 0.721 mmol), and BrettPhos Pd G3 precatalyst (0.653 g, 0.721 mmol) in 1,4-dioxane (280 mL) was added tert-butyl glycinate (7.39 mL, 54.1 mmol). The resulting suspension was degassed (5× vacuum/purge with nitrogen), and then heated to 90° C. After 16 hours, the reaction mixture was cooled to below 30° C., and additional BrettPhos Pd G3 precatalyst was added (0.653 g, 0.721 mmol). The reaction mixture was degassed (5× vacuum/purge with nitrogen), and then heating to 90° C. was resumed. After 7 hours, the reaction mixture was cooled to below 30° C., and additional BrettPhos Pd G3 precatalyst was added (0.653 g, 0.721 mmol). The reaction mixture was degassed (5× vacuum/purge with nitrogen), then heating to 90° C. was resumed. After 16 hours, the reaction mixture was cooled to below 30° C., and additional BrettPhos Pd G3 precatalyst was added (0.328 g, 0.362 mmol). The reaction mixture was degassed (5× vacuum/purge with nitrogen), then heating to 90° C. was resumed. After 4 hours, the reaction mixture was cooled to ambient temperature, quenched with saturated aqueous ammonium chloride (70 mL), and diluted with water (70 mL) and ethyl acetate (140 mL). The layers were separated, and the aqueous layer was extracted with ethyl acetate (2×70 mL). The organic layers were combined, washed with brine (42 mL), dried over anhydrous sodium sulfate and filtered. Silica (35 g) was added to the filtrate, and the mixture was concentrated in vacuo to a powder, which was dry loaded onto a 220 g gold Teledyne ISCO silica column, and purified by running a gradient of 0-40% ethyl acetate in heptanes with 0.1% triethylamine added to give 12.44 g (28.1 mmol, 78% yield) of the title compound. ¹H NMR (400 MHZ, CDCl₃) δ ppm 7.50-7.27 (m, 5H), 6.45 (d, J=1.4 Hz, 1H), 5.06 (s, 2H), 4.42 (s, 1H), 4.10-3.97 (m, 5H), 3.97-3.91 (m, 2H), 2.88 (t, J=6.8 Hz, 2H), 2.84 (s, 2H), 1.90 (t, J=6.6 Hz, 2H), 1.44 (s, 9H); MS (APCI+) m/z 444 [M+H]⁺.

### Example 5D: tert-butyl {[6′-(benzyloxy)-8′-fluoro-3′,4′-dihydro-1′H-spiro[[1,3]dioxolane-2,2′-naphthalen]-7′-yl]({[(prop-2-en-1-yl)oxy]carbonyl}sulfamoyl)amino}acetate

To a solution of chlorosulfonyl isocyanate (3.65 mL, 42.1 mmol) in dichloromethane (124 mL), was added allyl alcohol (2.86 mL, 42.1 mmol) dropwise. After 30 minutes, a preformed solution of the product of Example 5C (12.44 g, 28.1 mmol) and N,N-diisopropylethylamine (9.8 mL, 56.1 mmol) in dichloromethane (62 mL) was added slowly via an addition funnel. After 45 minutes, the reaction mixture was quenched with water (125 mL) and stirred for 5 minutes. The layers were separated, and the aqueous layer was extracted with dichloromethane (2×62 mL). The organic layers were combined, washed with 1 M aqueous sodium bisulfate (62 mL), dried over anhydrous sodium sulfate, filtered and concentrated in vacuo to give the title compound, which was used without purification for the next step. MS (APCI⁺) m/z 624 [M+NH₄]⁺.

### Example 5E: 5-[6′-(benzyloxy)-8′-fluoro-3′,4′-dihydro-1′H-spiro[[1,3]dioxolane-2,2′-naphthalen]-7′-yl]-1λ⁶,2,5-thiadiazolidine-1,1,3-trione

To a solution of the crude product of Example 5D (17.0 g, 28.1 mmol) in methanol (340 mL) was added tetrakis(triphenylphosphine)palladium(0) (0.648 g, 0.561 mmol), followed by a solution of sodium methoxide (38.5 mL, 25 weight % in methanol, 168 mmol). The resulting mixture was degassed (3× vacuum/nitrogen purge), and then heated to 60° C. After 1 hour, the reaction mixture was cooled to ambient temperature, quenched with 1 M hydrochloric acid (190 mL), diluted with ethyl acetate (85 mL) and partially concentrated in vacuo to remove methanol. The resulting biphasic mixture was extracted with ethyl acetate (3×85 mL). The organic layers were combined, washed with brine (51 mL), dried over anhydrous sodium sulfate, filtered through Celite® (5 g) and concentrated in vacuo. The residue was suspended in tert-butyl methyl ether (85 mL), heated to boiling, and then cooled to ambient temperature. The resulting solid was collected by filtration, washed with the cold filtrate and then with cold tert-butyl methyl ether (34 mL), and dried in a vacuum oven at 50° C. to give 7.95 g (17.72 mmol, 63.2% yield) of the title compound. MS (APCI⁺) m/z 449 [M+H]⁺.

### Example 5F: 5-[3-(benzyloxy)-1-fluoro-7-oxo-5,6,7,8-tetrahydronaphthalen-2-yl]-1λ⁶,2,5-thiadiazolidine-1,1,3-trione

The product from Example 5E (1.5 g, 3.34 mmol) was suspended in 88% formic acid (7.5 mL, 196 mmol). After 45 minutes, the reaction mixture was diluted with drop-wise addition of brine (15 mL). The resulting solid was collected by filtration, washed with water (4×7.5 mL) and dried in a vacuum oven at 50° C. to give 1.33 g (3.30 mmol, 99% yield) of the title compound. ¹H NMR (400 MHZ, DMSO-d₆) δ ppm 7.47 (d, J=6.8 Hz, 2H), 7.45-7.28 (m, 3H), 7.05 (s, 1H), 5.19 (s, 2H), 4.40 (s, 2H), 3.47 (s, 2H), 3.06 (t, J=6.7 Hz, 2H), 2.50 (t, J=6.7 Hz, 2H); MS (APCI+) m/z 422 [M+NH₄]⁺.

### Example 5G: 5-{3-(benzyloxy)-7-[(4,4-difluorobutyl)amino]-1-fluoro-5,6,7,8-tetrahydronaphthalen-2-yl}-1λ⁶,2,5-thiadiazolidine-1,1,3-trione

To a solution of the product of Example 5F (0.5 g, 1.24 mmol) in ethanol (10 mL) was added 4,4-difluorobutan-1-amine hydrochloride (0.270 g, 1.86 mmol) followed by triethylamine (0.517 mL, 3.71 mmol). After 30 minutes, sodium cyanoborohydride (0.093 g, 1.48 mmol) was added as a solid. The mixture was allowed to stir for 16 hours, and then was quenched with ammonium hydroxide (0.14 mL, 7.42 mmol) and diluted with acetonitrile (10 mL) and water (2 mL). Celite® (5 g) was added, and the mixture was concentrated in vacuo to give a powder. The resultant mixture was dry loaded onto a Teledyne ISCO 275 g reversed-phase C18 column eluted with a gradient of 10-100% methanol in buffer (0.025 M ammonium bicarbonate in water acidified to pH 7 by adding dry ice) to give the title compound (0.386 g, 0.776 mmol, 63% yield. MS (APCI⁺) m/z 498 [M+H]⁺.

### Example 5H: 5-{2-[(azetidin-3-yl)methyl]-8-fluoro-6-hydroxy-1,2,3,4-tetrahydroisoquinolin-7-yl}-1λ⁶,2,5-thiadiazolidine-1,1,3-trione

To a suspension of the product of Example 5G (0.386 g, 0.776 mmol) and pentamethylbenzene (0.230 g, 1.55 mmol) in dichloromethane (7.7 mL) at −78° C. was added a solution of boron trichloride (4.66 mL, 1 M in dichloromethane, 4.66 mmol) slowly along the side of the flask. The resulting mixture was stirred for 5 minutes, then warmed to an internal temperature of 0° C., then cooled to −78° C. and quenched with ethyl acetate (4 mL) followed by ethanol (4 mL). The reaction mixture was warmed to ambient temperature and concentrated in vacuo. The residue was triturated with heptanes (3× 8 mL), 1:1 ethyl acetate/heptanes (2×4 mL), dichloromethane (2×4 mL) and acetonitrile (3×4 mL), and then dried in a vacuum oven at 50° C. to give the title compound as an HCl salt (0.297 g, 0.669 mmol, 86% yield). ¹H NMR (400 MHZ, DMSO-d₆) δ ppm 10.17 (br s, 1H), 9.00 (br s, 2H), 6.54 (s, 1H), 6.15 (tt, J=56.6, 4.2 Hz, 1H), 4.32 (s, 2H), 3.48-3.40 (m, 1H), 316-3.02 (m, 3H), 2.88-2.70 (m, 2H), 2.61 (dd, J=16.1, 10.0 Hz, 1H), 2.24-2.16 (m, 1H), 2.09-1.86 (m, 2H), 1.84-1.67 (m, 3H); MS (APCI⁺) m/z 408 [M+H]⁺.

### Example 6: 5-{(7R)-7-[(2-cyclopentylethyl)amino]-1-fluoro-3-hydroxy-5,6,7,8-tetrahydronaphthalen-2-yl}-1λ⁶,2,5-thiadiazolidine-1,1,3-trione (Compound 200)

### Example 6A: 6-(benzyloxy)-7-bromo-8-fluoro-3,4-dihydronaphthalen-2(1H)-one

The product of Example 5B (33.31 g, 73.1 mmol) was suspended in 88% formic acid (70 mL). After 1.5 hours, the mixture was diluted with water (400 mL). The resulting solid was collected by filtration, washed with water (800 mL) and dried in a vacuum oven at 30° C. to give the title compound (26.84 g, 71.7 mmol, 98% yield) as a monohydrate. ¹H NMR (500 MHz, DMSO-d₆) δ ppm 7.49 (ddt, J=7.7, 1.4, 0.7 Hz, 2H), 7.45-7.39 (m, 2H), 7.37-7.32 (m, 1H), 7.09-7.05 (m, 1H), 5.23 (s, 2H), 3.50 (d, J=1.1 Hz, 2H), 3.08-3.01 (m, 2H), 2.48 (s, 2H).

### Example 6B: (2R)-6-(benzyloxy)-7-bromo-8-fluoro-1,2,3,4-tetrahydronaphthalen-2-amine hydrochloride

To a solution of monobasic sodium phosphate (38.2 g, 318 mmol) in water (0.95 L) was added concentrated hydrochloric acid (175 mL), followed by portion-wise addition of sec-butylamine (235 mL, 2326 mmol). The pH was adjusted to 6.5 by addition of concentrated hydrochloric acid. After cooling the mixture to 30° C. pyridoxal-5-phosphate (0.625 g, 286 mmol) was added, and 100 mL of the buffer solution was removed for use below. To the remaining buffer solution was slowly added a solution of the product of Example 6A (118 g, 338 mmol) in dimethyl sulfoxide (0.95 L) while maintaining the pH between 7.25 and 7.75 by addition of either concentrated hydrochloric acid or 50% aqueous sec-butylamine. Upon complete addition, a dispersion of Codexis® ATA-025 (12 g) in the 100 mL of buffer from above was added and the resulting mixture was heated to 40° C. while maintaining the pH between 7.25 and 7.75 by addition of either concentrated hydrochloric acid or 50% aqueous sec-butylamine. After 24 hours, the reaction mixture was cooled to 10° C. and filtered. The solid was triturated with water (2×250 mL) followed by acetonitrile (2×250 mL), and then dried in a vacuum oven at 40° C. to give the title compound (126 g, 91% potency by HPLC. 327 mmol, 96.9% potency adjusted yield). Analytical HPLC conditions; Supelco Acentis® Express C18 column. 4.6×150) mm. 2.7 micron, held at 35° C. eluting with a gradient of 30 to 90% acetonitrile in 0.1% perchloric acid in water over 6 minutes, holding at 90% acetonitrile for 1 minute then back to 30% acetonitrile over 0.1 minutes: ¹H NMR (500 MHz, CD₃OD) δ ppm 7.50-7.44 (m, 2H), 7.41-7.34 (m, 2H), 7.34-7.27 (m, 1H), 6.78-6.73 (m, 1H), 5.16 (d, J=3.8 Hz, 2H), 3.61-3.50 (m, 1H), 3.21 (ddt, J=16.1, 5.7, 1.7 Hz, 1H), 2.99-2.84 (m, 2H), 2.65 (dd, J=16.3, 9.8 Hz, 1H), 2.21 (dddd, J=14.5, 7.3, 4.2, 1.7 Hz, 1H), 1.84 (dddd, J=12.7, 11.1, 10.2, 6.3 Hz, 1H); MS (APCI+) m/z 350 [M+H]⁺.

### Example 6C: benzyl [(2R)-6-(benzyloxy)-7-bromo-8-fluoro-1,2,3,4-tetrahydronaphthalen-2-yl]carbamate

To a solution of the product of Example 6B (2 g, 5.17 mmol) in a mixture of tetrahydrofuran (20 mL) and water (10 mL) was added 1 M aqueous sodium hydroxide (10.35 mL, 10.35 mmol), followed by benzyl chloroformate (1.811 mL, 3 M in toluene, 5.43 mL) dropwise. After 10 minutes the reaction mixture was extracted with ethyl acetate (3×10 mL). The organic layers were combined, washed with brine (10 mL), dried over anhydrous sodium sulfate, filtered and concentrated. The residue was dissolved in boiling ethyl acetate (10 mL), and the solution was diluted by dropwise addition of heptanes (12 mL) and then slowly cooled to room temperature. The solid was collected by filtration, washed with 1:1 ethyl acetate/heptanes (10 mL) and dried in a vacuum oven at 50° C. to give the title compound (1.8513 g, 3.82 mmol, 74% yield). ¹H NMR (400 MHZ, DMSO-d₆) δ ppm 7.47-7.23 (m, 9H), 6.85 (s, 1H), 6.81 (s, 1H), 5.14 (s, 2H), 5.00 (s, 2H), 3.82-3.57 (m, 1H), 2.91 (dd, J=16.5, 5.3 Hz, 1H), 2.84-2.76 (m, 1H), 2.79-2.66 (m, 1H), 2.47-2.39 (m, 1H), 1.94-1.86 (m, 1H), 1.67-1.55 (m, 1H); MS (APCI⁺) m/z 484 [M+H]⁺.

### Example 6D: (R)-tert-butyl 2-((3-(benzyloxy)-7-(((benzyloxy)carbonyl)amino)-1-fluoro-5,6,7,8-tetrahydronaphthalen-2-yl)amino)acetate

To a suspension of the product of Example 6C (2.0876 g, 4.31 mmol), cesium carbonate (4.21 g, 12.93 mmol), BrettPhos (0.093 g, 0.172 mmol), and BrettPhos Pd G3 precatalyst (0.078 g, 0.086 mmol) in dioxane (41.8 mL) was added tert-butyl 2-aminoaceate (0.883 mL, 6.47 mmol). The resulting mixture was degassed by 5 vacuum/nitrogen backfills, stirred for 5 minutes and then heated to 90° C. After 3 hours, the mixture was cooled to below 30° C. BrettPhos Pd G3 precatalyst (0.078 g, 0.086 mmol) was added, and the mixture was degassed by 3 vacuum/nitrogen backfills and stirred for 5 minutes and then heated to 90° C. After 16 hours, the mixture was cooled to below 30° C., BrettPhos Pd G3 precatalyst (0.078 g, 0.086 mmol) was added, and the mixture was degassed by 3 vacuum/nitrogen backfills stirred for 5 minutes and then heated to 90° C. After 3.5 hours, the mixture was cooled to below 30° C. BrettPhos Pd G3 precatalyst (0.078 g, 0.086 mmol) was added, the mixture was degassed by 3 vacuum/nitrogen backfills stirred for 5 minutes and then heated to 90° C. After 3 hours, the reaction mixture was cooled to ambient temperature and quenched with saturated aqueous ammonium chloride (20 mL), diluted with water (10 mL) and extracted with ethyl acetate (20 mL, 2×10 mL). The organic layers were combined, washed with brine (10 mL), dried over anhydrous sodium sulfate, and filtered. Silica (10 g) was added to the filtrate, and the resulting mixture was concentrated in vacuo to give a yellow powder. The resultant mixture was dry loaded onto an 80 g Teledyne ISCO RediSep Rf Gold® column and eluted with a gradient of 0-35% ethyl acetate in heptanes with 0.1% triethylamine added to give the title compound (1.7647 g, 3.30 mmol, 77% yield). ¹H NMR (400 MHZ, DMSO-d₆) δ ppm 7.50-7.43 (m, 2H), 7.38 (s, 1H), 7.43-7.31 (m, 6H), 7.31 (s, 1H), 6.57 (s, 1H), 5.07 (s, 2H), 5.03 (s, 2H), 4.76 (td, J=6.8, 2.7 Hz, 1H), 3.88 (dd, J=6.9, 2.6 Hz, 2H), 3.69-3.57 (m, 1H), 2.86 (dd, J=16.4, 5.5 Hz, 1H), 2.75-2.66 (m, 2H), 2.36 (dd, J=16.5, 9.7 Hz, 1H), 1.95-1.87 (m, 1H), 1.60-1.48 (m, 1H), 1.34 (s, 9H); MS (APCI+) m/z 535 [M+H]⁺.

Early fractions gave the hydrodehalogenation by product benzyl [(2R)-6-(benzyloxy)-8-fluoro-1,2,3,4-tetrahydronaphthalen-2-yl]carbamate (0.1848 g, 0.456 mmol, 10.6% yield). ¹H NMR (500 MHZ, DMSO-d₆) δ ppm 7.49-7.39 (m, 3H), 7.42-7.34 (m, 6H), 7.37-7.28 (m, 2H), 6.68 (dd, J=11.5, 2.4 Hz, 1H), 6.62 (d, J=2.5 Hz, 1H), 5.06 (s, 2H), 5.03 (s, 2H), 3.69 (s, 1H), 2.90 (dd, J=16.5, 5.6 Hz, 1H), 2.80 (tt, J=16.6, 5.5 Hz, 2H), 2.39 (dd, J=16.6, 9.6 Hz, 1H), 1.93 (dd, J=12.7, 4.1 Hz, 1H), 1.59 (dtd, J=12.3, 10.5, 5.7 Hz, 1H); MS (APCI+) m/z 406 [M+H]⁺. Crystals suitable for X-ray crystallography of the hydrodehalogenation byproduct were grown from slow evaporation of a solution in methanol. X-ray crystallographic analysis confirmed the absolute stereochemistry to be (R).

### Example 6E: tert-butyl {[(7R)-3-(benzyloxy)-7-{[(benzyloxy)carbonyl]amino}-1-fluoro-5,6,7,8-tetrahydronaphthalen-2-yl]({[(prop-2-en-1-yl)oxy]carbonyl}sulfamoyl)amino}acetate

To a solution of chlorosulfonyl isocyanate (0.430 mL, 4.95 mmol) in dichloromethane (17.6 mL) at 0° C. was added allyl alcohol (0.337 mL, 4.95 mmol) dropwise. After 30 minutes, a preformed solution of the product of Example 6D (1.7647 g, 3.30 mmol), and N,N-diisopropylamine (1.73 mL, 9.90 mmol) in dichloromethane (17.6 mL) was slowly added along the side of the flask. After 45 minutes, the reaction was quenched with water (18 mL) and the layers were separated. The aqueous layer was extracted with dichloromethane (2×9 mL). The organic layers were combined and washed with 1 M aqueous sodium bisulfate (9 mL). The sodium bisulfate layer was extracted with dichloromethane (9 mL). The organic layers were combined and dried over anhydrous sodium sulfate, filtered and concentrated in vacuo to give the title compound which was used for the next reaction without purification. ¹H NMR (400 MHz, DMSO-d₆) δ ppm 11.49 (d, J=3.5 Hz, 1H), 7.47-7.24 (m, 10H), 6.73 (s, 1H), 5.69 (ddtd, J=17.4, 10.7, 5.5, 1.5 Hz, 1H), 5.23-5.05 (m, 3H), 5.07-4.95 (m, 6H), 4.59 (dd, J=17.3, 3.0 Hz, 1H), 4.28-4.18 (m, 1H), 4.21-4.07 (m, 2H), 3.74-3.54 (m, 1H), 2.87 (dd, J=16.7, 5.4 Hz, 1H), 2.81-2.68 (m, 1H), 2.38 (dd, J=16.5, 9.6 Hz, 1H), 1.97-1.87 (m, 1H), 1.29 (d, J=3.0 Hz, 9H); MS (APCI+) m/z 642 [M-tert-butyl+H]⁺.

### Example 6F; benzyl [(2R)-6-(benzyloxy)-8-fluoro-7-(1,1,4-trioxo-1λ⁶,2,5-thiadiazolidin-2-yl)-1,2,3,4-tetrahydronaphthalen-2-yl]carbamate

To a suspension of the product of Example 6E (2.306 g, 3.30 mmol) and tetrakis(triphenylphosphine)palladium(0) (0.076 g, 0.066 mmol) in methanol (23 mL) was added a solution of sodium methoxide (5.29 mL, 25 weight % in methanol, 23.13 mmol), and the resulting mixture was heated to 60° C. After 1.5 hours, the reaction mixture was cooled to ambient temperature, quenched 1 M hydrochloric acid (23 mL), diluted with ethyl acetate (23 mL) and partially concentrated to remove methanol. The crude aqueous mixture was extracted with 2-methyltetrahydrofuran (3×23 mL). The organic layers were combined, washed with brine (10 mL), dried over sodium sulfate, filtered through Celite® (5 g) and concentrated in vacuo. The residue was dissolved in acetonitrile (23 mL), Celite® (5 g) was added and the mixture was concentrated. The resulting mixture was dry loaded onto a 40 g Teledyne ISCO RediSep Rf Gold® column and eluted with a gradient of 0-100% acetonitrile in dichloromethane to give the title compound (1.3459 g, 2.494 mmol, 75% yield). ¹H NMR (600 MHZ, DMSO-d₆) δ ppm 7.51-7.39 (m, 3H), 7.39-7.34 (m, 6H), 7.34-7.28 (m, 2H), 6.82 (s, 1H), 5.12 (s, 2H), 5.08-5.00 (m, 2H), 4.38 (d, J=0.8 Hz, 2H), 3.74-3.70 (m, 1H), 2.95-2.75 (m, 3H), 2.44 (dd, J=16.6, 9.3 Hz, 1H), 1.97-1.91 (m, 1H), 1.62 (dtd, J=12.5, 10.4, 5.5 Hz, 1H); MS (APCI+) m/z 540 [M+H]⁺.

### Example 6G; benzyl [(2R)-6-(benzyloxy)-8-fluoro-7-(1,1,4-trioxo-1λ⁶,2,5-thiadiazolidin-2-yl)-1,2,3,4-tetrahydronaphthalen-2-yl](2-cyclopentylethyl)carbamate, ammonium salt

To a solution of the product of Example 6F (0.1 g, 0.185 mmol) in N,N-dimethylformamide (1 mL) was added potassium carbonate (0.026 g, 0.185 mmol) followed by (2-bromoethyl)cyclopentane (0.051 mL, 0.371 mmol). After stirring for 5 minutes, a suspension of potassium tert-butoxide (0.042 g, 0.371 mmol) in N,N-dimethylformamide (1 mL) was added dropwise over 30 minutes. After 90 minutes, additional (2-bromoethyl)cyclopentane (0.30 mL, 0.219 mmol) was added followed by a suspension of potassium tert-butoxide (0.042 g, 0.371 mmol) in N,N-dimethylformamide (1 mL) over 30 minutes. After 1 hour, the reaction mixture was diluted with water (1 mL) and filtered through a glass microfiber frit. The resulting solution was directly purified by loading onto a Teledyne ISCO 100 g reversed-phase C18 column eluted with a gradient of 10-100% methanol in buffer (0.025 M ammonium bicarbonate in water acidified to pH 7 by adding dry ice) to give the title compound (0.0501 g, 0.036 mmol, 41.4% yield). MS (APCI⁺) m/z 653 [M+NH₄]⁺.

### Example 6H: 5-{(7R)-7-[(2-cyclopentylethyl)amino]-1-fluoro-3-hydroxy-5,6,7,8-tetrahydronaphthalen-2-yl}-1λ⁶,2,5-thiadiazolidine-1,1,3-trione

To a suspension the product of Example 6G (0.0300 g, 0.061 mmol) and pentamethylbenzene (0.018 g, 0.123 mmol) in dichloromethane (1.2 mL) at −78° C. was added a solution of boron trichloride (0.368 mL, 0.368 mmol, 1 M in dichloromethane) slowly along the side of the flask. The resulting mixture was stirred for 5 minutes, then warmed to an internal temperature of 0° C., and then cooled to −78° C. and quenched with ethyl acetate (1 mL) followed by ethanol (1 mL). The reaction mixture was warmed to ambient temperature and concentrated in vacuo. The residue was triturated with heptanes (3×2 mL), 1:1 ethyl acetate/heptanes (2×2 mL), and dichloromethane (2×2 mL). The crude solid was dissolved in methanol (5 mL), Celite® (1 g) was added, and the mixture was concentrated. The resultant mixture was dry loaded onto a Teledyne ISCO 50 g reversed-phase C18 column and eluted with a gradient of 10-100% methanol in buffer (0.025 M ammonium bicarbonate in water acidified to pH 7 by adding dry ice) to give the title compound (0.0203 g, 0.049 mmol, 64.3% yield). ¹H NMR (400 MHZ, DMSO-d₆) δ ppm 9.21 (br s, 1H), 8.47 (br s, 2H), 6.47 (s, 1H), 3.93 (s, 2H), 3.45-3.38 (m, 1H), 3.09 (dd, J=16.0, 5.4 Hz, 1H), 3.01 (dd, J=10.0, 6.1 Hz, 2H), 2.85-2.65 (m, 2H), 2.57-2.46 (m, 1H), 2.16 (dd, J=11.5, 5.1 Hz, 1H), 1.88-1.42 (m, 9H), 1.18-1.05 (m, 2H); MS (APCI+) m/z 412 [M+H]⁺.

### Example 7: 5-[2-(aminomethyl)-4-fluoro-6-hydroxy-2,3-dihydro-1H-inden-5-yl]-1λ⁶,2,5-thiadiazolidine-1,1,3-trione (Compound 267)

### Example 7A: 5-(benzyloxy)-7-fluoro-2,3-dihydro-1H-inden-1-one

To a mixture of 5-bromo-7-fluoro-2,3-dihydro-1H-inden1-one (59 g, 232 mmol), water (20.88 mL, 1159 mmol) and cesium carbonate (177 g, 543 mmol) in N,N-dimethylformamide (600 mL) was added RockPhos Pd G3 precatalyst (1.944 g, 2.318 mmol) under N₂ at 25° C. The mixture was heated to 60° C. and stirred for 12 hours at 60° C. under N₂. Then the mixture was cooled to 25° C. Benzyl bromide (33.0 mL, 278 mmol) was added to the mixture, and the mixture was stirred for 2 hours at 25° C. One additional vial on a 59 g scale (with respect to 5-bromo-7-fluoro-2,3-dihydro-1H-inden1-one) was set up in parallel as described above. These two reaction mixtures were combined and diluted with water (2 L) and ethyl acetate (800 mL). Then the resulting mixture was filtered through diatomaceous earth. The two phases of the filtrate were cut, and the aqueous phase was extracted with ethyl acetate (2×800 mL). The combined organic phases were washed with brine (3×500 mL), dried over Na₂SO₄, and concentrated under reduced pressure. The residue was purified by column chromatography on silica gel eluted with ethyl acetate/petroleum ether (0-10%) to give the title compound (76 g, 267 mmol, 57.6% yield). ¹H NMR (400 MHZ, CDCl₃) δ ppm 7.46-7.35 (m, 5H), 6.81-6.76 (m, 1H), 6.61 (dd, J=10.88, 1.88 Hz, 1H), 5.13 (s, 2H), 3.12-3.06 (m, 2H), 2.73-2.67 (m, 2H).

### Example 7B: 5-(benzyloxy)-2-bromo-7-fluoro-2,3-dihydro-1H-inden-1-one

To a solution of the product from Example 7A (25 g, 88 mmol) in chloroform (125 mL) and ethyl acetate (125 mL) was added copper (II) bromide (23.53 g, 105 mmol) at 25° C. Then the mixture was stirred for 2 hours at 80° C. Then copper (II) bromide (23.53 g, 105 mmol) was added to the reaction mixture at 25° C. and the mixture was stirred for 2 hours at 80° C. One additional vial on a 20 g scale and one additional vial on 25 g scale were set up in parallel as described above. These three reaction mixtures were combined and filtered. The filtrate was concentrated under reduced pressure. The residue was triturated with 5:1 petroleum ether/ethyl acetate and filtered. The filter cake was the title compound. The filtrate was purified by flash column chromatography (10:1 petroleum ether/ethyl acetate) and combined with the filter cake to give the title compound (67.5 g, 181 mmol, 73.7% yield). ¹H NMR (400 MHZ, CDCl₃) δ ppm 7.47-7.35 (m, 5H), 6.78-6.74 (m, 1H), 6.67 (dd, J=10.63, 1.88 Hz, 1H), 5.19-5.12 (m, 2H), 4.62 (dd, J=7.50, 3.13 Hz, 1H), 3.78 (dd, J=18.39, 7.50 Hz, 1H), 3.37 (dd, J=18.39, 3.13 Hz, 1H).

### Example 70:5-(benzyloxy)-7-fluoro-1-oxo-2,3-dihydro-1H-indene-2-carbonitrile

To a solution of sodium cyanide (18.6 g, 380 mmol) in N,N-dimethylformamide (280) mL) and water (40 mL) was added a solution of the product from Example 7B (42.5 g, 114 mmol) in N,N-dimethylformamide (120 mL) dropwise at 0° C.) Then the mixture was stirred for 30 minutes at 25° C. One additional vial on a 1 g scale, one additional vial on a 2.8 g scale, one additional vial on a 7.6 g scale, and one additional vial on a 25 g scale were set up in parallel as described above. Then the mixture was diluted with water (4 L) and purified by reversed-phase column chromatography (Agela Claricep™ Flash AQ C18 Column, 20-35 μm. 100 Å, 800 g, flow rate 100 mL/minute, 0-100% gradient of acetonitrile in water, wavelength: 220 & 254 nm). The eluent was concentrated under reduced pressure to give crude product. The crude product was purified by column chromatography on silica gel eluted with tetrahydrofuran/petroleum ether (0-30%) to give the title compound (45.2 g, 145 mmol, 69.3% yield). ¹H NMR (400 MHZ, DMSO-d₆) δ ppm 7.52-7.31 (m, 6H), 7.09 (s, 1H), 7.01 (br d, J=11.51 Hz, 1H), 5.27 (s, 2H).

### Example 7D: 5-(benzyloxy)-7-fluoro-1-hydroxy-2,3-dihydro-1H-indene-2-carbonitrile

To a solution of the product from Example 7C (30 g, 96 mmol) in methanol (300 mL) and tetrahydrofuran (300 mL) was added sodium borohydride (5.45 g, 144 mmol) in portions at 0° C. Then the mixture was stirred for 2 hours at 25° C. Three additional vials on a 500 mg scale, one additional vial on a 5.7 g scale, and one additional vial on an 8 g scale were set up in parallel as described above. These six reactions were combined and quenched with water (1500 mL) and extracted with ethyl acetate (3×500 mL). The combined organic layers were washed with brine (300 mL), dried over Na₂SO₄, and concentrated under reduced pressure to give the crude product. The crude product was purified by column chromatography on silica gel eluted with petroleum ether/tetrahydrofuran (10:1 to 5:1, 10:1 byproduct, 5:1 product) to give the title compound (35 g, 111 mmol, 77% yield). ¹H NMR (400 MHZ, CDCl₃) δ ppm 7.41 (d, J=4.38 Hz, 5H), 6.67 (br d, J=5.38 Hz, 1H), 6.62 (dt, J=10.76, 2.44 Hz, 1H), 5.67 (t, J=4.63 Hz, 1H), 5.48 (t, J=5.38 Hz, 1H), 5.06 (s, 2H), 3.54-3.33 (m, 2H), 3.30-3.08 (m, 1H), 2.52 (d, J=4.88 Hz, 1H), 2.38 (d, J=5.38 Hz, 1H).

### Example 7E: 2-(aminomethyl)-7-fluoro-2,3-dihydro-1H-inden-5-ol hydrochloride

To a mixture of Pd—C (5 g, 4.70 mmol) in methanol (500 mL) and HCl (50 mL, 600 mmol) was added the product from Example 7D (10 g, 31.8 mmol) at 25° C. Then the mixture was stirred for 48 hours at 25° C. under H₂ (15 psi). One additional vial on a 10 g scale was set up in parallel as described above. These two reaction mixtures were combined and filtered through diatomaceous earth washed with methanol (1000 mL). The filtrate was evaporated under reduced pressure to give the title compound (13.7 g, 56.6 mmol, 89% yield), which was used directly for the next step. ¹H NMR (400 MHZ, DMSO-d₆) δ ppm 10.11-9.26 (m, 1H), 8.14 (br s, 3H), 6.49 (s, 1H), 6.36 (dd, J=10.88, 1.50 Hz, 1H), 2.83-3.02 (m, 4H), 2.77-2.55 (m, 3H).

### Example 7F: tert-butyl [(4-fluoro-6-hydroxy-2,3-dihydro-1H-inden-2-yl)methyl]carbamate

To a solution of the product from Example 7E (15.2 g, 62.8 mmol) in tetrahydrofuran (150 mL) and water (150 mL) was added sodium bicarbonate (26.4 g, 314 mmol) and then di-tert-butyl dicarbonate (21.89 mL, 94 mmol) was added dropwise at 0° C. Then the mixture was stirred for 12 hours at 25° C. One additional vial on a 500 mg scale and one additional vial on a 6 g scale were set up in parallel as described above. These three reactions were combined. The resulting mixture was diluted with water (500 mL) and extracted with ethyl acetate (3×200 mL). The combined organic phases were washed with brine (200 mL), dried over Na₂SO₄, and concentrated under reduced pressure. The crude product was purified by column chromatography on silica gel eluted with petroleum ether/ethyl acetate (20:1-5:1) to give the title compound (21.4 g, 72.3 mmol, 80% yield). ¹H NMR (400 MHZ, CDCl₃) δ ppm 6.48 (s, 1H), 6.38 (br d, J=10.13 Hz, 1H), 4.71 (br s, 1H), 3.29-3.14 (m, 1H), 3.29-3.14 (m, 1H), 3.05-2.91 (m, 2H), 2.75-2.50 (m, 3H), 1.46 (d, J=1.50 Hz, 10H).

### Example 7G; tert-butyl ({4-fluoro-6-[(2-methoxyethoxy)methoxy]-2,3-dihydro-1H-inden-2-yl}methyl)carbamate

To a solution of the product from Example 7F (5.6 g, 17.92 mmol) in anhydrous tetrahydrofuran (150) mL) was added cesium carbonate (8.76 g, 26.9 mmol) at 25° C. and then 2-methoxyethoxymethyl chloride (2.435 mL, 21.50 mmol) was added dropwise at 0° C.) Then the mixture was stirred for 1 hour at 0° C. Thin layer chromatography (phosphomolybdic acid, petroleum ether:ethyl acetate=3:1) showed 50% of starting material remained. Then cesium carbonate (5.84 g, 17.92 mmol) and 2-methoxyethoxymethyl chloride (2.029 mL, 17.92 mmol) were added to the mixture and stirred for 1 hour at 0° C. Thin layer chromatography (phosphomolybdic acid, petroleum ether:ethyl acetate=3:1) showed 50% of starting material still remained. One additional vial on a 1.3 g scale and one additional vial on a 5.6 g scale were set up in parallel as described above. These three reactions were combined. The combined reaction mixtures were diluted with water (600 mL) and extracted with ethyl acetate (3×200 mL). The combined organic phases were washed with brine (200 mL), dried over Na₂SO₄, and concentrated under reduced pressure to give the crude product. Two additional vials on a 2 g scale were set up as described above. The crude products were combined and purified by column chromatography on silica gel eluted with ethyl acetate/petroleum ether (10-12%) to give a mixture of the products of Examples 168F and 168G (18 g), which was used directly. To a solution of the products of Examples 168F and 168G (1 g, 3.55 mmol) in acetone (10 mL) was added cesium carbonate (1.737 g, 5.33 mmol) at 25° C. and then 2-methoxyethoxymethyl chloride (0.483 mL, 4.27 mmol) was added dropwise at 0° C. Then the mixture was stirred for 1 hour at 0° C. Seventeen additional vials on a 1 g scale were set up in parallel as described above. These reactions were combined. The resulting mixture was diluted with water (600 mL) and extracted with ethyl acetate (3×200 mL). The combined organic phases were washed with brine (200 mL), dried over Na₂SO₄, and concentrated under reduced pressure to give the crude product. The crude product was purified by column chromatography on silica gel eluted with ethyl acetate/petroleum ether (10-12%) to give the title compound (17 g, 41.4 mmol, 64.7% yield). ¹H NMR (400 MHz, CDCl₃) δ ppm 6.71 (s, 1H), 6.61-6.54 (m, 1H), 5.22 (s, 2H), 4.66 (br s, 1H), 3.81 (dd, J=5.50, 3.88 Hz, 2H), 3.57 (dd, J=5.44, 3.81 Hz, 3H), 3.39 (s, 3H), 3.20 (br d, J=5.50 Hz, 2H), 3.03 (br dd, J=15.51, 7.50 Hz, 2H), 1.46 (s, 9H), 2.55-2.75 (m, 3H).

### Example 7H; tert-butyl ({4-fluoro-5-iodo-6-[(2-methoxyethoxy)methoxy]-2,3-dihydro-1H-inden-2-yl}methyl)carbamate

To a solution from the product of Example 7G (5.9 g, 14.37 mmol) in anhydrous tetrahydrofuran (150 mL) was added n-butyllithium (34.5 mL, 86 mmol) dropwise at −70° C. under N₂. The mixture was stirred for 60 minutes at −70° C. under N₂. Then a solution of I₂ (23.71 g, 93 mmol) in tetrahydrofuran (30 mL) was added at −70° C. under N₂. The mixture was stirred for 60 minutes at −70° C. under N₂. Then the mixture was quenched with saturated NH₄Cl aqueous solution and saturated Na₂S₂O₃ aqueous solution (1:1, 500 mL) dropwise. The resulting solution was extracted with ethyl acetate (3×200 mL). The combined organic phases were washed with brine (200 mL), dried over Na₂SO₄, and concentrated under reduced pressure to give crude product. One additional vial on a 5 g scale and one additional vial on a 5.9 g scale were set up in parallel as described above. These crude products were combined and purified by column chromatography on silica gel eluted with ethyl acetate:petroleum ether=15-20% to give the title compound (14.5 g, 26.3 mmol, 64.3% yield). ¹H NMR (400 MHZ, CDCl₃) δ ppm 6.80 (s, 1H), 5.31 (s, 2H), 4.66 (br s, 1H), 3.93-3.82 (m, 2H), 3.63-3.54 (m, 2H), 3.39 (s, 3H), 3.26-3.13 (m, 2H), 3.12-2.99 (m, 2H), 2.75-2.61 (m, 3H), 1.46 (s, 9H).

### Example 7I: tert-butyl [(2-{[(tert-butoxycarbonyl)amino]methyl}-4-fluoro-6-[(2-methoxyethoxy)methoxy]-2,3-dihydro-1H-inden-5-yl)amino]acetate

To a solution of the product of Example 7H (2 g, 3.63 mmol) in dioxane (20 mL) was added cesium carbonate (3.55 g, 10.90 mmol) followed by tert-butyl 2-aminoacetate (1.430 g, 10.90 mmol) at 25° C. Then BrettPhos Pd G3 precatalyst (0.725 g, 0.799 mmol) was added under N₂. Then the mixture was stirred for 4 hours at 95° C. under N₂. One additional vial on a 735 mg scale and six additional vials on a 2 g scale were set up in parallel as described above. The resulting mixture was diluted with water (500 mL) and extracted with ethyl acetate (3×200 mL). The combined organic phases were washed with brine (200 mL), dried over Na₂SO₄, and concentrated under reduced pressure to give the crude product. The crude product was purified by column chromatography on silica gel eluted with ethyl acetate/petroleum ether=11-18%) to give the title compound (10 g, 19.05 mmol, 70.9% yield). ¹H NMR: (400 MHz, CDCl₃) δ ppm 6.77 (s, 1H), 5.26 (s, 2H), 4.64 (br s, 1H), 4.41 (br s, 1H), 3.95 (br s, 2H), 3.89-3.81 (m, 2H), 3.61-3.55 (m, 2H), 3.43-3.35 (m, 3H), 3.19 (br s, 2H), 3.07-2.87 (m, 2H), 2.73-2.50) (m, 3H), 1.46 (s, 18H).

### Example 7J: tert-butyl [(2-{[(tert-butoxycarbonyl)amino]methyl}-4-fluoro-6-[(2-methoxyethoxy)methoxy]-2,3-dihydro-1H-inden-5-yl)({[(prop-2-en-1-yl)oxy]carbonyl}sulfamoyl)amino]acetate

To a solution of chlorosulfonyl isocyanate (1.985 mL, 22.86 mmol) in dichloromethane (3 mL) was added allyl alcohol (1.555 mL, 22.86 mmol) dropwise at 0° C. The mixture was stirred for 30 minutes at 0° C. under N₂. Then the mixture was added to a mixture of the product of Example 7I (6 g, 11.43 mmol) and triethylamine (4.78 mL, 34.3 mmol) in dichloromethane (60 mL) dropwise at 0° C. The resulting mixture was stirred for 2 hours at 0° C. under N₂. Then the mixture was diluted with water (30 mL) and the organic phase was dried over Na₂SO₄ and concentrated under reduced pressure to give the title compound (8.5 g, 12.84 mmol, 112% yield), which was used directly for the next step. MS (ESI⁺) m/z 661 [M+23, M+46]⁺.

### Example 7K; tert-butyl ({4-fluoro-6-[(2-methoxyethoxy)methoxy]-5-(1,1,4-trioxo-1λ⁶,2,5-thiadiazolidin-2-yl)-2,3-dihydro-1H-inden-2-yl}methyl)carbamate

To a solution of the product of Example 7J (2.2 g, 3.32 mmol) in anhydrous methanol (22 mL) was added 4A molecular sieves (2.2 g). The resulting mixture was stirred for 10 minutes at 25° C. Then tetrakis(triphenylphosphine)palladium(0) (150 mg, 0.130 mmol) and sodium methoxide (4.31 g, 19.95 mmol) were added at 25° C. under N₂. The mixture was stirred for 2 hours at 60° C. under N₂. One additional vial on a 200 mg scale and one additional vial on a 2 g scale were set up in parallel as described above. These three reactions were combined. The combined mixture was filtered, and the filter cake was washed with water (100 mL) and methanol (20 mL). The filtrate was adjusted to pH=4 with aqueous HCl (1 mol/L) and extracted with ethyl acetate (3×50 mL). The combined organic phases were washed with a mixture of brine and aqueous HCl (1 mol/L) (4:1) (50 mL), dried over Na₂SO₄, and concentrated under reduced pressure to give the crude product. The crude product was purified by preparative HPLC [Shimadzu LC-8A preparative HPLC; Agela DuraShell C18 column, 250×70 mm×10 μm, flow rate 130 mL/minute, 20-40% in 20 minutes gradient of acetonitrile in water (10 mM NH₄HCO₃)]. To the product-containing eluent solution was added 1 M HCl (aqueous solution) to pH=4 and extracted with ethyl acetate (3×200 mL). The combined organic phases were washed with brine (200 mL), dried over Na₂SO₄ and concentrated under reduced pressure to give the title compound (1.5 g, 2.83 mmol, 42.6% yield). ¹H NMR (400 MHZ, DMSO-d₆) δ ppm 7.06-6.99 (m, 1H), 6.92 (s, 1H), 5.25 (s, 2H), 4.40 (s, 3H), 3.76-3.69 (m, 2H), 3.45 (dd, J=5.38, 4.00 Hz, 2H), 3.22 (s, 3H), 3.04-2.86 (m, 4H), 2.68-2.56 (m, 3H), 1.38 (s, 9H).

### Example 7L: 5-[2-(aminomethyl)-4-fluoro-6-hydroxy-2,3-dihydro-1H-inden-5-yl]-1λ⁶,2,5-thiadiazolidine-1,1,3-trione 2,2,2-trifluoroacetate

To a solution of the product of Example 7K (1.3 g, 2.453 mmol) in dichloromethane (18 mL) was added trifluoroacetic acid (6 mL, 78 mmol) dropwise at 0° C. The mixture was stirred for 2 hours at 25° C. One additional vial on a 100 mg scale was set up in parallel as described above. These two reactions were combined. The combined mixtures were evaporated under reduced pressure. The residue was triturated with methanol/water (3:1) to give the title compound (430) mg, 0.989 mmol, 31.5% yield) as a trifluoroacetate salt. ¹H NMR (400 MHZ, DMSO-d₆) δ ppm 9.13 (br s, 1H), 7.78 (br s, 3H), 6.57 (s, 1H), 3.96 (s, 2H), 3.05-2.86 (m, 4H), 2.77-2.58 (m, 3H); MS (ESI⁺) m/z 314 [M−H]⁻.

### Example 8: 5-[(3S)-3-amino-5-fluoro-7-hydroxy-3,4-dihydro-2H-1-benzopyran-6-yl]-1λ⁶,2,5-thiadiazolidine-1,1,3-trione (Compound 326)

### Example 8A: tert-butyl [(2S)-1-[4-(benzyloxy)-6-bromo-2-fluoro-3-(2,2,2-trifluoroacetamido)phenyl]-3-{[tert-butyl(dimethyl)silyl]oxy}propan-2-yl]carbamate

A solution of n-butyllithium in hexanes (1.91 M, 5.50 mL, 10.50 mmol, 2.1 equivalents) was added to a solution of diisopropylamine (1.57 mL, 11.00 mmol, 2.2 equivalents) in tetrahydrofuran (20.0 mL) at −78° C. The reaction mixture was stirred for 15 minutes at −78° C. A solution of the product of Example 1C (2.06 g, 5.25 mmol, 1.05 equivalents) in tetrahydrofuran (6.5 mL) was added dropwise via syringe pump over 20 minutes at −78° C. The reaction mixture was stirred for 30 minutes at −78° C. A solution of tert-butyl (R)-4-(((tert-butyldimethylsilyl)oxy)methyl)-1,2,3-oxathiazolidine-3-carboxylate 2,2-dioxide (1.84 g, 5.00 mmol, 1 equivalent; Tetrahedron Lett. 2011, 52, 5229-5233) in tetrahydrofuran (6.5 mL; 0.15 M overall) was added dropwise over 20 minutes at −78° C. The reaction mixture was stirred for 30 minutes at −78° C. Aqueous hydrochloric acid solution (3 M, 8.33 mL, 25.00 mmol, 5.0 equivalents) was added at −78° C. The resulting mixture was warmed over 20 minutes to 23° C. The warmed product mixture was diluted with ethyl acetate (100 mL). The resulting biphasic mixture was transferred to a separatory funnel and the layers that formed were separated. The aqueous layer was extracted with ethyl acetate (50 mL). The organic layers were combined and the combined organic layers were washed with saturated aqueous sodium chloride solution (20 mL). The washed organic layer was dried over sodium sulfate. The dried solution was filtered and the filtrate was concentrated. The residue obtained was dissolved in ether (20 mL). Diatomaceous earth (˜10 g) was added to the solution and the mixture was concentrated. The residue obtained was purified by flash-column chromatography (80 g RediSep Rf Gold” silica column, elution with a gradient from 0-80% ethyl acetate-heptanes) to furnish the title compound (1.977 g, 58%). MS (APCI⁺) m/z 579 [M+H—C(O)OC(CH₃)₃]⁺.

### Example 8B; methyl [{6-(benzyloxy)-4-bromo-3-[(2S)-2-[(tert-butoxycarbonyl)amino]-3-{[tert-butyl(dimethyl)silyl]oxy}propyl]-2-fluorophenyl}(trifluoroacetyl)amino]acetate

Methyl bromoacetate (0.22 mL, 2.43 mmol, 1.1 equivalents) was added to a suspension of the product of Example 8A (1.5 g, 2.21 mmol, 1 equivalent), potassium carbonate (915 mg, 6.62 mmol, 3.0 equivalents), and potassium iodide (183 mg, 1.10 mmol, 0.5 equivalent) in acetone (11 mL, 0.2 M) at 23° C. The reaction mixture was stirred for 24 hours at 23° C. The product mixture was concentrated. The residue obtained was partitioned between ethyl acetate (60 mL) and water (15 mL). The aqueous layer was extracted with ethyl acetate (30 mL). The organic layers were combined and washed with saturated aqueous sodium chloride solution (15 mL). The washed organic layer was dried over sodium sulfate. The dried solution was filtered. The titled compound obtained was used without further purification in the following step. MS (APCI⁺) ma: 651 [M+H—C(O)OC(CH₃)₃]⁺.

### Example 8C: methyl {[6-(benzyloxy)-4-bromo-3-{(2S)-2-[(tert-butoxycarbonyl)amino]-3-hydroxypropyl}-2-fluorophenyl](trifluoroacetyl)amino}acetate

A solution of tetrabutylammonium fluoride in tetrahydrofuran (1 M, 7.70 mL, 7.70 mmol, 1.1 equivalents) was added to a solution of the product of Example 8B (nominally 7 mmol, 1 equivalent) in tetrahydrofuran (35 mL, 0.2 M) at 23° C. The reaction mixture was stirred for 4 days at 23° C. The product mixture was partitioned between ethyl acetate (150 mL), water (25 mL), and saturated aqueous ammonium chloride solution (25 mL). The aqueous layer was extracted with ethyl acetate (50 mL). The organic layers were combined and the combined organic layers were washed with saturated aqueous sodium chloride solution (20 mL). The washed solution was dried over sodium sulfate. The dried solution was filtered. Diatomaceous earth (˜3 g) was added to the filtrate and the mixture was concentrated. The residue obtained was purified by flash-column chromatography (120 g RediSep Rf Gold® silica column, elution with a gradient from 0-100% ethyl acetate-heptanes) to furnish the title compound (4.41 g, 99%, two steps). MS (APCI⁺) m/z 637 [M+H]⁺.

### Example 8D: methyl [{(3S)-7-(benzyloxy)-3-[(tert-butoxycarbonyl)amino]-5-fluoro-3,4-dihydro-2H-1-benzopyran-6-yl}(trifluoroacetyl)amino]acetate

A suspension of the product of Example 8C (4.46 g, 7.00 mmol, 1 equivalent), potassium phosphate tribasic (4.46 g, 21.00 mmol, 3.0 equivalents), palladium(II)acetate (79.0 mg, 0.35 mmol, 5.0 mol %), and [1,1′-binaphthanlen]-2-yldi-tert-butylphosphine (TrixiePhos, 122 mg, 0.49 mmol, 7.0 mol %) in toluene (35 mL, 0.2 M) was sealed in a 100 mL round-bottom flask outfitted with a rubber septum and nitrogen inlet. The sealed reaction mixture was deoxygenated by iterative subjections to vacuum (˜5 seconds) and subsequent backfilling with nitrogen (×3). The reaction vessel was placed in a heating block that had been preheated to 90° C. The reaction mixture was stirred for 45 minutes at 90° C. The product mixture was then cooled to 23° C. The cooled product mixture was partitioned between water (50 mL) and ethyl acetate (150 mL). The aqueous layer was extracted with ethyl acetate (100 mL). The organic layers were combined and the combined organic layers were washed with saturated aqueous sodium chloride solution (50 mL). The washed organic layer was dried over sodium sulfate. The dried solution was filtered. Diatomaceous earth (˜15 g) was added to the filtrate and the mixture was concentrated. The residue obtained was purified by flash-column chromatography (330 g RediSep Rf Gold® silica column, elution with a gradient from 0-100% ethyl acetate-heptanes). The fractions containing product were collected and concentrated. The residue obtained was purified by flash-column chromatography (120 g RediSep Rf Gold® silica column, elution with a gradient from 0-100% ethyl acetate-heptanes) to furnish the title compound (1.98 g, 51%). ¹H NMR (400 MHZ, CDCl₃) δ ppm 7.44-7.32 (m, 5H), 6.32 (s, 1H), 5.02 (s, 2H), 4.75 (bs, 1H), 4.63 (d, J=16.8 Hz, 1H), 4.23-4.03 (m, 3H), 3.95 (dd, J=16.8, 4.4 Hz, 1H), 3.65 (s, 3H), 2.99-2.87 (m, 1H), 2.67 (ddd, J=16.0, 10.7, 4.6 Hz, 1H), 1.45 (s, 9H); MS (APCI⁺) m/z 574 [M+NH₄]⁺.

### Example 8F; methyl ({(3S)-7-(benzyloxy)-3-[(tert-butoxycarbonyl)amino]-5-fluoro-3,4-dihydro-2H-1-benzopyran-6-yl}amino)acetate

A solution of sodium methoxide in methanol (0.5 M, 9.80 mL, 4.93 mmol, 3.1 equivalents) was added to a solution of the product of Example 8D (885 mg, 1.59 mmol, 1 equivalent) in anhydrous methanol (10.00 mL, 0.16 M) under nitrogen at 23° C. The reaction vessel was outfitted with a reflux condenser equipped with a rubber septum and nitrogen inlet. The vessel was immediately placed in a heating block that had been preheated to 65° C. The reaction mixture was stirred for 24 hours at 65° C. The product mixture was then cooled to 23° C. The cooled product mixture was concentrated. The residue obtained was partitioned between aqueous hydrochloric acid solution (1.0 M, 8 mL) and ethyl acetate (30 mL). The aqueous layer was extracted with ethyl acetate (2×10 mL). The organic layers were combined and the combined organic layers were washed with saturated aqueous sodium chloride solution (5 mL). The washed organic layer was dried over sodium sulfate. The dried solution was filtered. Diatomaceous earth (˜4.5 g) was added to the solution and the mixture was concentrated. The residue obtained was purified by flash-column chromatography (40 g RediSep Rf Gold® silica column, elution with a gradient from 0-100% ethyl acetate-heptanes) to furnish the title compound (345 mg, 47%), MS (APCI⁺) m/z 461 [M+H]⁺.

### Example 8F: methyl [{(3S)-7-(benzyloxy)-3-[(tert-butoxycarbonyl)amino]-5-fluoro-3,4-dihydro-2H-1-benzopyran-6-yl}({[(prop-2-en-1-yl)oxy]carbonyl}sulfamoyl)amino]acetate

Allyl alcohol (0.06 mL, 0.90 mmol, 1.2 equivalents) was added to a solution of chlorosulfonyl isocyanate (0.07 mL, 0.82 mmol, 1.1 equivalents) in dichloromethane (1.00 mL) at 23° C. The reaction mixture was stirred for 30 minutes at 23° C. A solution of the product of Example 8E (354 mg, 0.75 mmol, 1 equivalent) and diisopropylethylamine (0.26 mL, 1.50 mmol, 2.0 equivalents) in dichloromethane (2.00 mL, 0.2 M overall) was added slowly at 23° C. The reaction mixture was stirred for 18 hours at 23° C. The product mixture was partitioned between saturated aqueous ammonium chloride solution (3 mL) and ethyl acetate (15 mL). The aqueous layer was extracted with ethyl acetate (10 mL). The organic layers were combined and the combined organic layers were washed with saturated aqueous sodium chloride solution (5 mL). The washed organic layer was dried over sodium sulfate. The dried solution was filtered, and the filtrate was concentrated. The residue obtained was used without further purification in the following step. MS (APCI⁺) m/z 641 [M+NH₄]⁺.

### Example 8G; tert-butyl [(3S)-7-(benzyloxy)-5-fluoro-6-(1,1,4-trioxo-1λ⁶,2,5-thiadiazolidin-2-yl)-3,4-dihydro-2H-1-benzopyran-3-yl]carbamate, ammonia salt

A solution of sodium methoxide in methanol (0.5 M, 3.84 mL, 1.92 mmol, 3.0 equivalents) was added to a suspension of the product of Example 8F (nominally 0.64 mmol, 1 equivalent) and tetrakis(triphenylphosphine)palladium(0) (37 mg, 0.03 mmol, 0.05 equivalents) in anhydrous methanol (2.0 mL, 0.32 M) under nitrogen at 23° C. The reaction was sealed and the sealed reaction mixture was deoxygenated by iterative subjections to vacuum (˜5 seconds) and subsequent backfilling with nitrogen (×3). The reaction vessel was placed in a heating block that had been preheated to 60° C. The reaction mixture was stirred for 10 minutes at 60° C. The product mixture was then cooled to 23° C. The cooled mixture was diluted with aqueous hydrochloric acid solution (3.0 M, 1.0 mL). The diluted mixture was partially concentrated under a stream of nitrogen. The partially concentrated mixture was partitioned between ethyl acetate (25 mL) and saturated aqueous ammonium chloride solution (5 mL). The aqueous layer was extracted with ethyl acetate (10 mL). The organic layers were combined and the combined organic layers were washed with saturated aqueous sodium chloride solution. The washed organic layer was dried over sodium sulfate. The dried solution was filtered, and the filtrate was concentrated. The residue obtained was purified by reverse phase flash column chromatography (100 g RediSep Rf Gold® C18 column, elution with a gradient of 5-100% methanol-0.025 M aqueous ammonium bicarbonate solution [acidified with solid carbon dioxide]) to furnish the title compound (110 mg, 29% over two steps). ¹H NMR (400 MHZ, DMSO-d₆) δ ppm 7.48 (apparent d, J=7.0 Hz, 2H), 7.36-7.26 (m, 3H), 6.35 (s, 1H), 5.09 (s, 2H), 4.08 (apparent d, J=8.3 Hz, 1H), 3.91 (s, 2H), 3.83-3.67 (m, 2H), 2.81 (dd, J=17.1, 4.9 Hz, 1H), 1.39 (s, 9H); MS (APCI+) m/z 525 [M+NH₄]⁺.

### Example 8H; tert-butyl [(3S)-5-fluoro-7-hydroxy-6-(1,1,4-trioxo-1λ⁶,2,5-thiadiazolidin-2-yl)-3,4-dihydro-2H-1-benzopyran-3-yl]carbamate

A suspension of palladium-on-carbon (10% weight, 17.8 mg, 0.017 mmol, 10 mol %), ammonium formate (53.0 mg, 0.84 mmol, 5.0 equivalents), and the product of Example 8G (85.0 mg, 0.17 mmol, 1 equivalent) in ethanol (1.3 mL, 0.13 M) was stirred for 1 hour at 60° C. The product mixture was cooled to 23° C. The cooled product mixture was diluted with methanol (1.5 mL) and filtered through a plug of diatomaceous earth (0.5 cm×1.0 cm). The filter cake was rinsed with methanol (3×1.5 mL). The filtrates were combined, and the combined filtrates were concentrated. The residue obtained was used without further purification in the following step. MS (APCI⁺) m/z 435 [M+NH₄]⁺.

### Example 8I: 5-[(3S)-3-amino-5-fluoro-7-hydroxy-3,4-dihydro-2H-1-benzopyran-6-yl]-1λ⁶,2,5-thiadiazolidine-1,1,3-trione, ammonia salt

Trifluoroacetic acid (0.3 mL, 3.91 mmol, 19.0 equivalents) was added to a suspension of the product of Example 8H (nominally 0.206 mmol, 1 equivalent) in dichloromethane (0.70 mL, ˜0.3 M) at 23° C. The reaction mixture was stirred for 1 hour at 23° C. The product mixture was then diluted with diethyl ether (2.0 mL). The diluted product mixture was concentrated. The residue obtained was purified by reverse phase flash column chromatography (50 g RediSep Rf Gold® C18 column, elution with a gradient of 5-100% methanol-0.025 M aqueous ammonium bicarbonate solution [acidified with solid carbon dioxide]) to furnish the title compound (34.0 mg, 49% over two steps). ¹H NMR (400 MHZ, DMSO-d₆) δ ppm 6.19 (s, 1H), 4.13 (d, J=11.1 Hz, 1H), 4.03 (dd, J=11.3, 5.1 Hz, 1H), 3.90 (s, 2H), 3.69-3.63 (m, 1H), 2.96 (dd, J=16.8, 5.7 Hz, 1H), 2.58 (dd, J=16.7, 4.3 Hz, 1H); MS (APCI+) m/z 318 [M+H]⁺.

### Example 9: 5-{(7R)-1-fluoro-3-hydroxy-7-[(3-methylbutyl)amino]-5,6,7,8-tetrahydronaphthalen-2-yl}(4,4-²H₂)-1λ⁶,2,5-thiadiazolidine-1,1,3-trione (Compound 327)

### Example 9A; benzyl [(2R)-6-(benzyloxy)-7-bromo-8-fluoro-1,2,3,4-tetrahydronaphthalen-2-yl](3-methylbutyl)carbamate

To a suspension of the product of Example 6B (127 g, 328 mmol) in a mixture of dichloromethane (1.5 L), and ethanol (1.0 L) was added triethylamine (46.4 g, 459 mmol) and after 3 minutes, 3-methylbutanal (36.7 mL, 426 mmol) was added. The suspension was stirred at room temperature for 2 hours after which sodium borohydride (31.0 g, 252 mmol) was added carefully portionwise (caution: gas evolution!). After 10 minutes, the reaction was quenched via slow addition of methanol (120 mL) over 10 minutes followed by water (1.2 L) over 20 minutes. The resulting biphasic suspension was separated, the aqueous layer was extracted with dichloromethane (1×400 mL), and the combined organic extracts were washed with brine (1×1 L), dried over sodium sulfate, filtered, and concentrated to afford 140 g of (2R)-6-(benzyloxy)-7-bromo-8-fluoro-N-(3-methylbutyl)-1,2,3,4-tetrahydronaphthalen-2-amine that was used in the subsequent reaction without further purification.

To a solution of the crude (2R)-6-(benzyloxy)-7-bromo-8-fluoro-N-(3-methylbutyl)-1,2,3,4-tetrahydronaphthalen-2-amine in a mixture of tetrahydrofuran (1.2 L) and water (600 mL) was added 1 M aqueous sodium hydroxide (303 mL, 303 mmol) in one portion followed by neat benzyl chloroformate (49.1 g, 288 mmol) slowly over 5 minutes. After 10 minutes, additional benzyl chloroformate (4.14 g, 24.2 mmol) was added, and the reaction was judged to be complete. The mixture was partitioned between water (700 mL) and ethyl acetate (2×300 mL). The combined organic extracts were washed with brine (2×300 mL), dried over sodium sulfate, filtered, diluted with heptanes (400 mL), and concentrated. The crude residue (163 g) was dissolved in toluene (1 L), silica gel (182 g) was added, the suspension was stirred vigorously for 20 minutes and subsequently filtered onto a bed of diatomaceous earth (250 g) topped with silica gel (200 g) that had been pre-equilibrated with heptanes (750 mL) and toluene (250 mL). After filtration, the pad was washed with additional toluene/ethyl acetate (10:1, 2×200 mL), and the volatiles were removed in vacuo to afford the title compound (152.6 g, 275 mmol, 84% yield over 2 steps). ¹H NMR (400 MHZ, CDCl₃) δ ppm 7.55-7.30 (m, 10H), 6.49 (s, 1H), 5.18 (ABq, 2H), 5.13 (s, 2H), 4.17 (br, 1H), 3.22 (br s, 2H), 2.98 (dd, J=16.4, 5.6 Hz, 1H), 2.86 (br s, 2H), 2.73 (dd, J=16.3, 11.6 Hz, 1H), 1.94 (m, 2H), 1.53 (m, 2H), 1.31 (m, 1H), 0.91 (m, 6H); MS (APCI+) m/z 556 [M+H]⁺.

### Example 9B; benzyl {(2R)-6-(benzyloxy)-7-[(tert-butoxycarbonyl)amino]-8-fluoro-1,2,3,4-tetrahydronaphthalen-2-yl}(3-methylbutyl)carbamate

Benzyl [(2R)-6-(benzyloxy)-7-bromo-8-fluoro-1,2,3,4-tetrahydronaphthalen-2-yl](3-methylbutyl)carbamate (300 mg, 0.541 mmol, Example 9A), tert-butyl carbamate (127 mg, 1.082 mmol), BrettPhos Pd G3 (49.0 mg, 0.054 mmol), BrettPhos (29.0 mg, 0.054 mmol), and cesium carbonate (353 mg, 1.082 mmol) were sealed in a vial, and the vial was degassed with nitrogen. 1,4-Dioxane (2 mL) was added to the reaction vial, and the reaction mixture was degassed again with nitrogen. The reaction mixture was stirred at 90° C. for 8.5 hours, then cooled to ambient temperature. The reaction was quenched with aqueous 0.2 M HCl (2 mL), diluted with brine (10 mL), and extracted with ethyl acetate. The combined organic layers were dried over sodium sulfate, filtered, and concentrated under reduced pressure. The residue was purified by flash column chromatography (40 g RediSep Rf Gold® silica gel column, 5-40% gradient of ethyl acetate/heptanes, flow rate=40 mL/minute) to give the title compound (228.7 mg, 0387 mmol, 71.6%), MS (ESI⁺) m/z 608 [M+NH₄]⁺.

### Example 9C: benzyl [(2R)-7-amino-6-(benzyloxy)-8-fluoro-1,2,3,4-tetrahydronaphthalen-2-yl](3-methylbutyl)carbamate

Trifluoroacetic acid (0.5 mL, 6.49 mmol) was added to a solution of the product of Example 9B (136.1 mg, 0.230 mmol) in dichloromethane (2 mL). The reaction mixture was stirred at ambient temperature for 1 hour. Then the reaction mixture was washed with aqueous saturated sodium bicarbonate (10 mL). The aqueous layer was then extracted with dichloromethane. The combined organic layers were dried over sodium sulfate, and filtered.

The filtrate was concentrated under reduced pressure to give the title compound that was used directly for the next reaction. MS (ESI⁺) m/z 491 [M+H]⁺.

### Example 9D: methyl {[(7R)-3-(benzyloxy)-7-{[(benzyloxy)carbonyl](3-methylbutyl)amino}-1-fluoro-5,6,7,8-tetrahydronaphthalen-2-yl]amino}(²H₂)acetate

Methyl bromo(²H₂)acetate (25 μL, 0.264 mmol) was added to a solution of the product of Example 9C (102.8 mg, 0.210 mmol) and potassium carbonate (121 mg, 0.876 mmol) in acetonitrile (6 mL) and N,N-dimethylformamide (1 mL). The reaction mixture was stirred at 60° C. for 3 hours. The temperature was lowered to 50° C. and additional methyl bromo(²H₂)acetate (50 μL, 0.528 mmol) was added, and the mixture was stirred further for 21 hours at 50° C. The reaction was quenched with 3% v/v CH₃CO₂D in D₂O (1 mL). The mixture was extracted with ethyl acetate, and the organic fraction was washed with saturated aqueous ammonium chloride. The organic layers were dried over sodium sulfate and filtered, and the filtrate was concentrated under reduced pressure to give the title compound. The title compound was used without further purification in the next reaction. MS (ESI⁺) m/z 565 [M+H]⁺.

### Example 9E; methyl {[(7R)-3-(benzyloxy)-7-{[(benzyloxy)carbonyl](3-methylbutyl)amino}-1-fluoro-5,6,7,8-tetrahydronaphthalen-2-yl][(tert-butoxycarbonyl)sulfamoyl]amino}(²H₂)acetate

tert-Butanol (0.040 mL, 0.420 mmol) was added to a solution of chlorosulfonyl isocyanate (0.036 mL, 0.420 mmol) in dichloromethane (1.5 mL) at 0° C., and the mixture was stirred for 30 minutes at 0° C. Then a solution of the product of Example 9D and triethylamine (0.088 mL, 0.630 mmol) in dichloromethane (1.5 mL) was added. The reaction mixture stirred at ambient temperature for 45 minutes. The reaction mixture was quenched with D₂O (1 mL) and extracted with dichloromethane. The organic layers were combined, dried over sodium sulfate, and filtered, and the filtrate was concentrated under reduced pressure to give the title compound. The title compound was used without further purification in the next reaction. MS (ESI⁺) m/z 761 [M+NH₄]⁺.

### Example 9F: methyl {[(7R)-3-(benzyloxy)-7-{[(benzyloxy)carbonyl](3-methylbutyl)amino}-1-fluoro-5,6,7,8-tetrahydronaphthalen-2-yl](sulfamoyl)amino}(²H₂)acetate

Trifluoro(²H)acetic acid (0.170 mL, 2.206 mmol) was added to a solution of the product of Example 9E in dichloromethane (2 mL). The solution was stirred at ambient temperature for 1.5 hours, additional trifluoro(²H)acetic acid (200 μL) was added, and the solution was stirred further for 45 minutes at ambient temperature. Then more trifluoro(²H)acetic acid (400 μL) was added and stirred further for 45 minutes at ambient temperature. The reaction mixture was concentrated under reduced pressure and the title compound was used without further purification in the next reaction. MS (ESI⁺) m/z 661 [M+NH₄]⁺

### Example 9G: benzyl {(2R)-6-(benzyloxy)-8-fluoro-7-[1,1,4-trioxo(3,3-²H₂)-1λ⁶,2,5-thiadiazolidin-2-yl]-1,2,3,4-tetrahydronaphthalen-2-yl}(3-methylbutyl)carbamate

Potassium carbonate (290 mg, 2.100 mmol) was added to a solution of the product of Example 9F in (²H₃)methan(²H)ol (2 mL), and the mixture was stirred for 5 minutes. Then sodium hydride (16.80 mg, 0.420 mmol) was added and the mixture was stirred at ambient temperature for 30 minutes. Additional sodium hydride (25.2 mg, 0.630 mmol) was added followed by stirring for a further 30 minutes at ambient temperature. The reaction was quenched with a solution of DCI (133 μL, 35% weight in D₂O) dissolved in D₂O up to 1.5 mL followed by DCI (100 μL, 35% weight in D₂O). The solution was extracted with ethyl acetate and the combined organic layers were dried over sodium sulfate and filtered, and the filtrate was concentrated under reduced pressure. The residue was purified by reverse phase column chromatography (60 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=50 mL/minute) to afford the title compound (39.4 mg, 0.064 mmol, 30.7% yield over 5 steps). MS (ESI⁺) m/z 629 [M+NH₄]⁺.

### Example 9H: 5-{(7R)-1-fluoro-3-hydroxy-7-[(3-methylbutyl)amino]-5,6,7,8-tetrahydronaphthalen-2-yl}(4,4-²H₂)-1λ⁶,2,5-thiadiazolidine-1,1,3-trione

The product of Example 9G (38.9 mg, 0.064 mmol) and tetrahydrofuran (4 mL) were added to 5% Pd/C (60 mg, 0.263 mmol) in a 20 mL Barnstead STEM RS10 with a glass liner. The reaction mixture was stirred at 25° C. under an atmosphere of hydrogen at 112.19-114.51 psi for 19 hours and 20 minutes. The catalyst was then removed by filtration and washed with methanol. The filtrate was concentrated under reduced pressure and purified by reverse phase column chromatography (60 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=50 mL/minute) to afford the title compound (13.5 mg, 0.035 mmol, 54.8% yield, 90% deuterium incorporation). ¹H NMR (400 MHZ, DMSO-d₆) δ ppm 9.20 (s, 1H), 8.38 (s, 2H), 6.47 (s, 1H), 3.47-3.41 (m, 1H), 3.10 (dd, J=16.1, 5.4 Hz, 1H), 3.03 (t, J=8.2 Hz, 2H), 2.85-2.67 (m, 3H), 2.19-2.11 (m, 1H), 1.72-1.56 (m, 2H), 1.50 (q, J=7.2 Hz, 2H), 0.92 (d, J=6.6 Hz, 6H); MS (ESI⁺) m/z 388 [M+H]⁺.

### Example 10: 8-fluoro-6-hydroxy-N-(2-methylpropyl)-7-(1,1,4-trioxo-1λ⁶,2,5-thiadiazolidin-2-yl)-3,4-dihydroisoquinoline-2(1H)-sulfonamide (Compound 328)

### Example 10A: 6-(benzyloxy)-8-fluoro-N-(2-methylpropyl)-7-(1,1,4-trioxo-1λ⁶,2,5-thiadiazolidin-2-yl)-3,4-dihydroisoquinoline-2(1H)-sulfonamide

To a suspension of the product of Example 4A (50 mg, 0.089 mmol) in tetrahydrofuran (2 mL) were successively added triethylamine (0.05 mL, 0.359 mmol) and isobutylsulfamoyl chloride (0.02 mL, 0.147 mmol). The resulting mixture was allowed to stir at room temperature for 5.5 hours, then quenched with a saturated aqueous solution of ammonium chloride (3 mL). The mixture was concentrated to dryness on diatomaceous earth. The crude residue was subjected to column chromatography (Büchi Reveleris® C18, dry load with diatomaceous earth. 10-100% methanol in 0.1% ammonium bicarbonate) to afford the title compound (46 mg, 0.079 mmol, 88% yield. 90% purity). ¹H NMR (400 MHz, DMSO-d₆) δ ppm 7.52-7.42 (m, 3H), 7.37-7.26 (m, 3H), 7.11 (s, 1H), 6.79 (s, 1H), 5.12 (s, 2H), 4.17 (s, 2H), 3.95 (s, 2H), 3.37 (t, J=5.8 Hz, 2H), 2.82 (t, J=5.9 Hz, 2H), 2.69 (t, J=6.4 Hz, 2H), 1.67 (hept, J=6.7 Hz, 1H), 0.85 (d, J=6.7 Hz, 6H); MS (ESI⁺) m/z 527 [M+H]⁺.

### Example 10B: 8-fluoro-6-hydroxy-N-(2-methylpropyl)-7-(1,1,4-trioxo-1λ⁶,2,5-thiadiazolidin-2-yl)-3,4-dihydroisoquinoline-2(1H)-sulfonamide

To a solution of the product of Example 10A (46 mg, 0.070 mmol) in water (2 mL) was added 10% Pd/C (10 mg). The resulting suspension was allowed to stir under hydrogen (1.5 bar) for 1 hour. Additional 10% Pd/C (10 mg) and ethanol (0.5 mL) were added, and the suspension was stirred under hydrogen for an additional 2 hours. The mixture was subjected to column chromatography (Büchi Reveleris® C18, 10-100% methanol in 0.1% ammonium bicarbonate) to afford the title compound as an ammonium salt (25 mg, 0.053 mmol, 76% yield). ¹H NMR (400 MHz, DMSO-d₆) δ ppm 7.44 (s, 1H), 7.13 (s, 1,5H), 6.51 (s, 1H), 4.13 (s, 2H), 3.94 (s, 2H), 3.38-3.28 (m, 2H), 2.78 (t, J=5.9 Hz, 2H), 2.69 (t, J=5.8 Hz, 2H), 1.68 (hept, J=13.6, 6.7 Hz, 1H), 0.86 (d, J=6.6 Hz, 6H); MS (ESI⁺) m/z 437 [M+H]⁺.

### Example 11: 8-fluoro-6-hydroxy-N-(2-methylpropyl)-7-(1,1,4-trioxo-1λ⁶,2,5-thiadiazolidin-2-yl)-3,4-dihydroisoquinoline-2(1H)-carboximidamide (Compound 329)

### Example 11A: 5-[6-(benzyloxy)-8-fluoro-1,2,3,4-tetrahydroisoquinolin-7-yl]-1λ⁶,2,5-thiadiazolidine-1,1,3-trione

To a suspension of the product of Example 4A (260 mg, 0.631 mmol) in N,N-dimethylformamide (3 mL) were successively added triethylamine (0.1 mL, 0.717 mmol) and N-isobutylcyanamide (85 mg, 0.820 mmol). The resulting mixture was allowed to stir in a microwave (CEM, Discover®, 100 W) at 90° C. for 2 hours. The reaction was cooled to room temperature and additional N-isobutylcyanamide (52 mg, 0.503 mmol) was added. The mixture was allowed to stir in a microwave at 90° C. for an additional 2 hours. The mixture was cooled down to room temperature concentrated in vacuo. The crude residue was subjected to column chromatography (Büchi Reveleris® C18, dry load with diatomaceous earth, 20-80% methanol in 0.1% ammonium bicarbonate in water) to afford the title compound (126 mg, 0.245 mmol, 39% yield). ¹H NMR (400 MHZ, DMSO-d₆) δ ppm 7.80-7.75 (m, 1H), 7.70 (s, 1H), 7.52-7.47 (m, 2H), 7.39-7.32 (m, 2H), 7.32-7.27 (m, 1H), 6.87 (s, 1H), 5.15 (s, 2H), 4.51 (s, 2H), 3.96 (s, 2H), 3.61 (t, J=5.6 Hz, 2H), 3.02 (t, J=6.4 Hz, 2H), 2.91-2.84 (m, 2H), 1.92-1.77 (m, 1H), 0.89 (d, J=6.6 Hz, 6H); MS (ESI⁺) m/z 490 [M+H]⁺.

### Example 11B: 8-fluoro-6-hydroxy-N-(2-methylpropyl)-7-(1,1,4-trioxo-1λ⁶,2,5-thiadiazolidin-2-yl)-3,4-dihydroisoquinoline-2(1H)-carboximidamide

To a suspension of the product of Example 11A (126 mg, 0.245 mmol) in water (2 mL) and ethanol (6 mL) was added 10% Pd/C (26 mg). The resulting mixture was allowed to stir under hydrogen (1.5 bar) for 3 hours. The mixture was concentrated in vacuo and the crude residue was subjected to column chromatography (Büchi Reveleris® C18, dry load with diatomaceous earth, 10-100% methanol in 0.1% ammonium hydroxide) to afford the title compound (53 mg, 0.126 mmol, 52% yield). ¹H NMR (400 MHZ, DMSO-d₆) δ ppm 9.41 (s, 1H), 7.74 (s, 1H), 7.68 (s, 1H), 6.57 (s, 1H), 4.46 (s, 2H), 3.94 (s, 2H), 3.58 (t, J=5.7 Hz, 2H), 3.06-2.98 (m, 2H), 2.83 (t, J=5.8 Hz, 2H), 1.92-1.79 (m, 1H), 0.89 (d, J=6.6 Hz, 6H); MS (ESI⁺) m/z 400.3 [M+H]⁺.

### Example 12: 5-(1-fluoro-3-hydroxy-7-{[2-(oxetan-3-yl)ethyl]amino}-5,6,7,8-tetrahydronaphthalen-2-yl)-1λ⁶,2,5-thiadiazolidine-1,1,3-trione (Compound 330)

### Example 12A: 5-[3-(benzyloxy)-1-fluoro-7-{[2-(oxetan-3-yl)ethyl]amino}-5,6,7,8-tetrahydronaphthalen-2-yl]-1λ⁶,2,5-thiadiazolidine-1,1,3-trione

A solution of the product of Example 5F (60 mg, 0.144 mmol), 2-(oxetan-3-yl)ethanamine (23 mg, 0.227 mmol) and acetic acid (0.02 mL, 0.349 mmol) in dichloromethane (1 mL) was stirred at room temperature for 15 minutes before adding sodium triacetoxy borohydride (47 mg, 0.222 mmol). The resulting solution was stirred at room temperature for 24 hours. Sodium bicarbonate (50 mg, 0.595 mmol) was added, and the mixture was concentrated in vacuo. The crude residue was subjected to column chromatography (Büchi Reveleris® C18, dry loaded with diatomaceous earth, 5-50% acetonitrile in 10 mM ammonium bicarbonate) to afford the title compound (38 mg, 0.074 mmol, 51% yield). ¹H NMR (400 MHz, DMSO-d₆) δ ppm 9.83-9.40 (m, 1H), 7.54-7.46 (m, 2H), 7.39-7.32 (m, 2H), 7.32-7.26 (m, 1H), 6.75 (s, 1H), 5.12 (s, 2H), 4.82 (s, 1H), 3.95 (s, 2H), 3.68 (s, 2H), 3.58-3.33 (m, 4H), 2.97 (s, 1H), 2.93-2.75 (m, 2H), 2.74-2.63 (m, 1H), 2.30-2.19 (m, 1H), 2.19-1.93 (m, 1H), 1.91-1.70 (m, 2H), 1.70-1.57 (m, 1H); MS (ESI⁺) m/z 490 [M+H]⁺.

### Example 12B: 5-(1-fluoro-3-hydroxy-7-{[2-(oxetan-3-yl)ethyl]amino}-5,6,7,8-tetrahydronaphthalen-2-yl)-1λ⁶,2,5-thiadiazolidine-1,1,3-trione

A mixture of the product of Example 12A (25 mg, 0.050 mmol) and 10% palladium on carbon (10 mg, 9.40 μmol) in water (3 mL) and methanol (1.5 mL) was hydrogenated at 1 bar for 2 hours. The mixture was filtered through a pad of diatomaceous earth and washed with methanol (20 mL). The filtrate was concentrated in vacuo. The crude residue was subjected to column chromatography (Büchi Reveleris® C18, 0-30% methanol in 0.1% ammonium hydroxide) to afford the title compound (3 mg, 7.14 μmol, 14% yield). ¹H NMR (400 MHZ, DMSO-d₆) δ ppm 9.70 (s, 1H), 9.22 (s, 1H), 6.46 (s, 1H), 4.91 (s, 1H), 3.93 (s, 2H), 3.83-3.56 (m, 2H), 3.39 (s, 4H), 3.17-3.00 (m, 1H), 2.88-2.64 (m, 1H), 2.59 (s, 1H), 2.37 (s, 1H), 2.28-2.05 (m, 2H), 1.95 (s, 1H), 1.69 (s, 3H); MS (ESI⁺) m/z 400 [M+H]⁺.

### Example 13: 5-{(7R)-1,4-difluoro-3-hydroxy-7-[(3-methylbutyl)amino]-5,6,7,8-tetrahydronaphthalen-2-yl}-1λ⁶,2,5-thiadiazolidine-1,1,3-trione (Compound 331)

### Example 13A; benzyl [(2R)-6-(benzyloxy)-8-fluoro-7-(1,1,4-trioxo-1λ⁶,2,5-thiadiazolidin-2-yl)-1,2,3,4-tetrahydronaphthalen-2-yl](3-methylbutyl)carbamate

To a solution of the product of Example 6F (0.25 g, 0.463 mmol) in N,N-dimethylformamide (2.5 mL) was added potassium tert-butoxide (0.156 g, 1.390 mmol) at 0° C.) After 15 minutes, 1-bromo-3-methylbutane (0.09 mL, 0.720 mmol) was added dropwise, and the mixture was stirred at room temperature for 16 hours. Additional potassium tert-butoxide (0.052 g, 0.463 mmol) as a solid was added to the reaction mixture. After 5 minutes, additional 1-bromo-3-methylbutane (0.06 mL, 0.480 mmol) was added dropwise at room temperature and the mixture was stirred at room temperature for 3 more hours. The reaction mixture was then cooled to 0° C., quenched with 1 M HCl (3 mL), and extracted with ethyl acetate (3×3 mL). The combined organic layers were washed with brine (1 mL), dried over sodium sulfate, and filtered. Diatomaceous earth (ca. 3 g) was added to the filtrate, and the mixture was concentrated. The residue was purified by chromatography on a 100 g C18 Teledyne ISCO RediSep® Rf Gold column eluted with a gradient of 10-100% methanol in buffer (0.025 M ammonium bicarbonate in water, modified to pH 7 with dry ice) to give the title compound as an ammonium salt (0.0934 g, 32% yield). ¹H NMR (400 MHZ, DMSO-d₆) δ ppm 7.52 7.45 (m, 2H), 7.43-7.25 (m, 8H). 6.71 (s, 1H), 5.15-5.03 (m, 4H), 4.06-4.00 (m, 1H), 3.97 (s, 2H), 3.26-3.18 (m, 2H), 2.87-2.79 (m, 2H), 2.78-2.63 (m, 2H), 1.97-1.84 (m, 2H), 1.55-1.32 (m, 3H), 0.92-0.82 (m, 6H).

### Example 13B; benzyl [(2R)-6-(benzyloxy)-5,8-difluoro-7-(1,1,4-trioxo-1λ⁶,2,5-thiadiazolidin-2-yl)-1,2,3,4-tetrahydronaphthalen-2-yl](3-methylbutyl)carbamate

To a solution of the product of Example 13A (100 mg, 0.164 mmol) in acetonitrile (5 mL) was added Selectfluor® (1-(chloromethyl)-4-fluoro-1,4-diazoniabicyclo[2,2,2]octane; ditetrafluoroborate, 116 mg, 0.328 mmol) in portions at 20° C. under nitrogen and the mixture was stirred for 2 hours at 40° C. An additional three batches of Selectfluor® (116 mg, 0.328 mmol) were added every two hours at 40° C. The resulting mixture was stirred for an additional 2 hours at 40° C. under nitrogen before it was quenched with a saturated aqueous solution of Na₂S₂O₄ (10 mL). The reaction mixture was extracted with ethyl acetate (3×10 mL). The combined organic layers were dried over Na₂SO₄, filtrated and most of the volatiles were removed by concentration with a stream of nitrogen. The residue was purified by preparative HPLC on a Phenomenex® Gemini-NX C18 75×30 mm, 3 μm column eluted with acetonitrile-10 mM NH₄HCO₃ in H₂O with a gradient 20-50% for 5 minutes and 50-100% for 2 minutes) at a flow rate of 40 mL/minute to give the titled compound (12 mg, 0.017 mmol, purity 90%, yield 10.49%) after lyophilization. ¹H NMR (400 MHZ, DMSO-d₆) δ ppm 7.47 (br d, J=6.48 Hz, 2H) 7.30-7.41 (m, 7H), 5.06-5.15 (m, 4H), 3.94-4.09 (m, 3H), 3.16-3.28 (m, 3H), 2.96 (br dd, J=18.40, 2.38 Hz, 1H), 2.96 (br dd, J=18.40, 2.38 Hz, 1H), 2.70-2.86 (m, 3H), 1.83-2.03 (m, 2H), 1.31-1.60 (m, 3H), 0.85 (br s, 6H).

### Example 13C: 5-{(7R)-1,4-difluoro-3-hydroxy-7-[(3-methylbutyl)amino]-5,6,7,8-tetrahydronaphthalen-2-yl}-1λ⁶,2,5-thiadiazolidine-1,1,3-trione

To a solution of the product of Example 13B (8 mg, 0.011 mmol, purity 90%) in tetrahydrofuran (5 mL) was added Pd/C (5 mg, 4.70 μmol, 10%) in tetrahydrofuran (2 mL) under N₂. The mixture was stirred under H₂ (15 psi) at 20° C. for 12 hours. The mixture was then filtered, and the solids were washed with tetrahydrofuran (20 mL). The filtrate was concentrated with a stream of nitrogen and the crude residue was purified by preparative HPLC on a Waters Xbridge BEH C18 column (100×30 mm, 10 μm, eluted with acetonitrile-10 mM NH₄HCO₃ in H₂O with a gradient 1-35% for 8 minutes and 35-100% for 2 minutes) at a flow rate of 40 mL/minute to give the title compound (1.5 mg, yield 21%) after lyophilization. 1H NMR (400 MHZ, methanol-d₄) δ ppm 4.59 (s, 3H), 4.26 (s, 2H), 3.43-3.55 (m, 1H), 3.01-3.18 (m, 3H), 2.71-2.86 (m, 1H), 2.62 (dd, J=16.26, 10.03 Hz, 1H), 2.28-2.40 (m, 1H), 1.67-1.88 (m, 2H), 1.01 (d, J=6.48 Hz, 6H); MS (ESI⁺) m/z 404 [M−H]⁻.

### Example 14; N-[8-fluoro-6-hydroxy-7-(1,1,4-trioxo-1λ⁶,2,5-thiadiazolidin-2-yl)-1,2,3,4-tetrahydronaphthalen-2-yl]-3-methylbutane-1-sulfonamide (Compound 332)

### Example 14A: 5-[7-amino-3-(benzyloxy)-1-fluoro-5,6,7,8-tetrahydronaphthalen-2-yl]-1λ⁶,2,5-thiadiazolidine-1,1,3-trione

To a stirred mixture of the product of Example 5F (400 mg, 0.940 mmol) and ammonium acetate (762 mg, 9.89 mmol) in dioxane (6 mL) was added acetic acid (0.040 mL, 0.699 mmol). The reaction mixture was stirred at room temperature for 30 minutes. Sodium triacetoxy borohydride (841 mg, 3.97 mmol) was added and the reaction mixture was stirred for 16 hours. The mixture was cooled in an ice bath, quenched with water (0.7 mL), and concentrated in vacuo. The crude residue was subjected to column chromatography (Büchi Reveleris® C18, dry load with diatomaceous earth, 5-50% acetonitrile in 10 mM ammonium bicarbonate) to afford the title compound (138 mg, 0.323 mmol, 34% yield). ¹H NMR (500 MHz, DMSO-d₆) δ ppm 7.96 (s, 3H), 7.52-7.47 (m, 2H), 7.35 (d, J=7.2 Hz, 2H), 7.29 (t, J=7.3 Hz, 1H), 6.74 (s, 1H), 5.11 (s, 2H), 3.95 (d, J=1.3 Hz, 2H), 3.46 (s, 1H), 3.03 (dd, J=16.2, 5.5 Hz, 1H), 2.80 (d, J=12.6 Hz, 2H), 2.54 (d, J=9.5 Hz, 1H), 2.05 (d, J=10.5 Hz, 1H), 1.71 (dt, J=17.6, 11.0 Hz, 1H); MS (ESI⁺) m/z 406 [M+H]⁺.

### Example 14B; N-[6-(benzyloxy)-8-fluoro-7-(1,1,4-trioxo-1λ⁶,2,5-thiadiazolidin-2-yl)-1,2,3,4-tetrahydronaphthalen-2-yl]-3-methylbutane-1-sulfonamide

To a solution of the product of Example 14A (100 mg, 0.247 mmol) in tetrahydrofuran (4 mL) and N,N-dimethylformamide (2 mL) were successively added triethylamine (0.08 mL, 0.574 mmol) and 3-methylbutane-1-sulfonyl chloride (0.04 mL, 0.281 mmol). The resulting mixture was stirred at room temperature for 4 hours. Additional 3-methylbutane-1-sulfonyl chloride (0.04 mL, 0.281 mmol) and triethylamine (0.08 mL, 0.574 mmol) were added. The reaction mixture was stirred for another 18 hours. The reaction mixture was diluted with a saturated aqueous solution of sodium hydrogen carbonate (3 mL) and the mixture was concentrated in vacuo. The crude residue was subjected to column chromatography (Büchi Reveleris® C18, dry load with diatomaceous earth, 10-100% methanol in 0.1% ammonium bicarbonate) to afford the title compound (16 mg, 0.021 mmol, 8% yield, 70% purity). ¹H NMR (400 MHZ, DMSO-d₆) δ ppm 7.55-7.47 (m, 1H), 7.39-7.23 (m, 4H), 7.02 (s, 3H), 6.70 (s, 1H), 5.10 (s, 2H), 3.94 (s, 2H), 3.54 (s, 1H), 3.10-3.01 (m, 2H), 3.00-2.88 (m, 1H), 2.87-2.71 (m, 2H), 2.01-1.93 (m, 1H), 1.74-1.52 (m, 4H), 0.91 (d, J=6.5 Hz, 6H); MS (ESI⁻) m/z 538 [M−H]⁻.

### Example 14C: N-[8-fluoro-6-hydroxy-7-(1,1,4-trioxo-1λ⁶,2,5-thiadiazolidin-2-yl)-1,2,3,4-tetrahydronaphthalen-2-yl]-3-methylbutane-1-sulfonamide, 0.7ammonium salt

To a solution of the product of Example 14B (16 mg, 0.030 mmol) in water (1 mL) and ethanol (1 mL) was added 10% Pd/C (3 mg). The resulting suspension was allowed to stir under hydrogen (1.5 bar) for 2 hours. The suspension was subjected to column chromatography (Büchi Reveleris® C18, 10-100% methanol in 0.1% ammonium bicarbonate) to afford the title compound as a partial ammonium salt (4 mg, 7.81 μmol, 26% yield, 90% purity). ¹H NMR (500 MHz, DMSO-d₆) δ ppm 7.23 (d, J=7.0 Hz, 1H), 6.96 (s, 3H), 6.43 (s, 1H), 3.92 (s, 2H), 3.51 (s, 1H), 3.08-3.01 (m, 2H), 2.90 (dd, J=16.7, 5.8 Hz, 1H), 2.81-2.67 (m, 2H), 2.43 (dd, J=16.4, 9.3 Hz, 1H), 1.99-1.92 (m, 1H), 1.72-1.52 (m, 4H), 0.90 (d, J=6.6 Hz, 6H); MS (ESI⁻) m/z 448 [M−H]⁻.

### Example 15: 5-(1-fluoro-3-hydroxy-7-{[(2-methylpropyl)amino]methyl}-5,6,7,8-tetrahydronaphthalen-2-yl)-1λ⁶,2,5-thiadiazolidine-1,1,3-trione (Compound 333)

### Example 15A; ethyl [6-(benzyloxy)-7-bromo-8-fluoro-3,4-dihydronaphthalen-2(1H)-ylidene]acetate

To a solution of ethyl 2-(diethoxyphosphoryl)acetate (13.87 g, 61.9 mmol) in 1,2-dimethoxyethane (200 mL) was added NaH (2.474 g, 61.9 mmol, purity 60%) in portions at 0° C.) under nitrogen. The mixture was stirred at 0° C., for 1 hour. Then a solution of the product of Example 6A (20 g, 51.5 mmol, purity 90%) in 1,2-dimethoxyethane (200 mL) was added to the above solution dropwise at 0° C. The mixture was stirred at 0° C. for 1 hour. Thin-layer chromatography (petroleum ether:ethyl acetate=3:1, R_{f}=0.6) showed starting material was consumed. The mixture was quenched with saturated aqueous NH₄Cl (200 mL) and extracted with ethyl acetate (3×300 mL). The organic fraction was washed with brine (500 mL), dried over anhydrous sodium sulfate, and filtered, and the filtrate was concentrated under reduced pressure. The residue was purified by flash column chromatography on silica gel (petroleum ether:ethyl acetate=20:1) to give the title compound (19.7 g, 42.3 mmol, purity 90%, yield 82%), MS (ESI⁻) m/z 417 [M−H]⁻.

### Example 15B; ethyl (7-bromo-8-fluoro-6-hydroxy-1,2,3,4-tetrahydronaphthalen-2-yl)acetate

To a mixture of platinum (IV) oxide (3.02 g, 13.31 mmol) in methanol (5 mL) was added a solution of the product of Example 15A (6.2 g, 13.31 mmol, purity 90%) in tetrahydrofuran (30 mL) and methanol (30 mL) at 20° C. The mixture was stirred at 20° C. for 12 hours under H₂ (15 psi). Thin-layer chromatography (petroleum ether:ethyl acetate=3:1, R_{f}=0.25) showed starting material was consumed. One additional vial on a 3.2 g scale was set up as described above in parallel. The reaction mixtures were combined and filtered through a pad of diatomaceous earth, and the filtrate was concentrated under reduced pressure to give the title compound (9 g, 21.74 mmol, purity 80%) which used directly without further purification in the next step. MS (ESI⁻) m/z 329 [M−H]⁻.

### Example 15C: ethyl [6-(benzyloxy)-7-bromo-8-fluoro-1,2,3,4-tetrahydronaphthalen-2-yl]acetate

To a solution of the product of Example 15B (8.2 g, 19.81 mmol, purity 80%) in N,N-dimethylformamide (90 mL) was added cesium carbonate (12.91 g, 39.6 mmol) followed by (bromomethyl)benzene (5.08 g, 29.7 mmol) at 20° C. The mixture was stirred at 40° C. for 1 hour. Thin-layer chromatography (petroleum ether:ethyl acetate=3:1, R_{f}=0.8) showed starting material was consumed. The mixture was quenched with water (80 mL) and extracted with ethyl acetate (3×70 mL). The organic phase was dried over anhydrous sodium sulfate and filtered, and the filtrate was concentrated under reduced pressure. The residue was purified by flash column chromatography on silica gel (petroleum ether:ethyl acetate=20:1) to give the title compound (9 g, 19.23 mmol, purity 90%, yield 95.5% two steps). MS (ESI⁺) m/z 421 [M+H]⁺.

### Example 15D: [6-(benzyloxy)-7-bromo-8-fluoro-1,2,3,4-tetrahydronaphthalen-2-yl]acetic acid

To a solution of the product of Example 15C (10 g, 21.36 mmol, purity 90%) in tetrahydrofuran (40 mL), methanol (40 mL), and water (20 mL) was added LiOH (2.56 g, 107 mmol) at 20° C. The mixture was stirred at 20° C. for 12 hours. Thin-layer chromatography (petroleum ether:ethyl acetate=3:1, R_{f}=0.05) showed starting material was consumed and desired product was detected. The mixture was adjusted to pH=3 with aqueous 1 M HCl and the mixture was extracted with ethyl acetate (3×100 mL). The combined organic fractions were washed with brine (500 mL), dried over anhydrous sodium sulfate, and filtered. The filtrate was concentrated under reduced pressure to give the title compound (7.5 g, 17.16 mmol, purity 90%, yield 80%), which was used without further purification in the next step. MS (ESI⁻) m/z 391 [M−H]⁻.

### Example 15E: [6-(benzyloxy)-7-bromo-8-fluoro-1,2,3,4-tetrahydronaphthalen-2-yl]acetyl azide

To a solution the product of Example 15D (14 g, 35.6 mmol, purity 90%) in tetrahydrofuran (140 mL) was added triethylamine (10.92 mL, 78 mmol) at 0° C.) Then ethyl carbonochloridate (5.80 g, 53.4 mmol) was added dropwise to the above solution at 0° C.) The mixture was stirred at 0° C. for 1 hour. A solution of sodium azide (3.94 g, 60.5 mmol) in water (10 mL) was added to the above mixture dropwise at 0° C. The mixture was stirred at 0° C. for 1 hour. The mixture was quenched with water (60 mL) and extracted with ethyl acetate (3×150 mL). The combined organic phases were dried over anhydrous sodium sulfate and filtered, and the filtrate was concentrated under reduced pressure to give the title compound (14 g, 33.5 mmol, purity 80%, yield 94%), which was used directly in the next step without further purification.

### Example 15F: tert-butyl {[6-(benzyloxy)-7-bromo-8-fluoro-1,2,3,4-tetrahydronaphthalen-2-yl]methyl}carbamate

To a solution of the product of Example 15E (2.5 g, 5.38 mmol, purity 90%) in toluene (20 mL) was added 2-methylpropan-2-ol (20 mL) at 20° C. The mixture was stirred at 130° C. for 12 hours. Thin-layer chromatography (petroleum ether:ethyl acetate=3:1, R_{f}=0.45) showed starting material was consumed. The mixture was diluted with water (40 mL) and extracted with ethyl acetate (3×50 mL). The combined organic phases were dried over anhydrous sodium sulfate and filtered, and the filtrate was concentrated under reduced pressure to give the title compound (1.5 g, 2.91 mmol, purity 90%, yield 54%), which was used directly in the next step. ¹H NMR (400 MHZ, DMSO-d₆) δ ppm 7.51-7.30 (m, 5H), 6.50 (s, 1H), 5.12 (s, 2H), 4.73-4.65 (m, 1H), 3.27-3.07 (m, 2H), 2.90 (br dd, J=5.1, 16.3 Hz, 1H), 2.82-2.65 (m, 2H), 2.24 (br dd, J=10.3, 16.8 Hz, 1H), 2.00-1.77 (m, 2H), 1.47 (s, 9H), 1.42-1.32 (m, 1H).

### Example 15G; tert-butyl {[3-(benzyloxy)-7-{[(tert-butoxycarbonyl)amino]methyl}-1-fluoro-5,6,7,8-tetrahydronaphthalen-2-yl]amino}acetate

To a solution of the product of Example 15F (1.5 g, 2.91 mmol, purity 90%) in 1,4-dioxane (30 mL) was added cesium carbonate (1.894 g, 5.81 mmol), tert-butyl 2-aminoacetate (0.458 g, 3.49 mmol), and BrettPhos Pd G3 (0.264 g, 0.291 mmol) at 20° C. under nitrogen. The mixture was stirred at 95° C. for 12 hours under nitrogen. The mixture was diluted with water (50 mL) and extracted with ethyl acetate (3×50 mL). The organic fraction was dried over anhydrous sodium sulfate and filtered, and the filtrate was concentrated under reduced pressure to give the title compound (1.9 g), which was used in the next step without further purification. MS (ESI⁺) m/z 515 [M+H]⁺.

### Example 15H; tert-butyl {[3-(benzyloxy)-7-{[(tert-butoxycarbonyl)amino]methyl}-1-fluoro-5,6,7,8-tetrahydronaphthalen-2-yl]({[(prop-2-en-1-yl)oxy]carbonyl}sulfamoyl)amino}acetate

To a solution of sulfurisocyanatidic chloride (0.784 g, 5.54 mmol) in methylene chloride (20 mL) was added allyl alcohol (0.322 g, 5.54 mmol) dropwise at 0° C. The mixture was stirred at 0° C. for 1 hour. Then a solution of the product of Example 15G (1.9 g, crude) and N,N-diisopropylethylamine (1.612 mL, 9.23 mmol) in methylene chloride (20 mL) was added dropwise to the above solution at 0° C. The mixture was stirred at 0° C. for 1 hour. Thin-layer chromatography (petroleum ether:ethyl acetate=3:1, R_{f}=0.4) showed starting material was consumed. The mixture was quenched with water (50 mL) and extracted with ethyl acetate (3×40 mL). The combined organic phases were washed with brine (60 mL), dried over anhydrous sodium sulfate, and filtered. The filtrate was concentrated under reduced pressure. The residue was purified by flash column chromatography (petroleum ether:ethyl acetate=2:1) to give the title compound (800 mg, 0.472 mmol, purity 40%, yield 16% over two steps). MS (ESI⁻) m/z 676 [M−H]⁻.

### Example 15I: tert-butyl {[6-(benzyloxy)-8-fluoro-7-(1,1,4-trioxo-1λ⁶,2,5-thiadiazolidin-2-yl)-1,2,3,4-tetrahydronaphthalen-2-yl]methyl}carbamate

To a solution of the product from Example 15H (500 mg, 0.295 mmol, purity 40%) in methanol (10 mL) was added tetrakis(triphenylphosphine)palladium(0)) (34.1 mg, 0.03 mmol) and sodium methoxide (266 mg, 1.475 mmol) at 20° C. under nitrogen. The mixture was stirred at 60° C. for 6 hours under nitrogen. The mixture was diluted with water (20 mL) and extracted with ethyl acetate (3×20 mL). The combined organic fraction was washed with brine (60 mL), dried over anhydrous sodium sulfate, and filtered. The filtrate was concentrated under reduced pressure. The residue was purified by HPLC [Gilson 281 semi-preparative HPLC, Welch Xtimate C18 column, 100×25 mm×3 μm, flow rate=25 mL/minute, 50-100% acetonitrile in water (0.4% v/v HCl/H₂O]. Product containing fractions were lyophilized to give the title compound (24 mg, 0.043 mmol, purity 94%, 14.71% yield). ¹H NMR (400 MHZ, DMSO-d₆) δ ppm 7.44 (d, J=6.7 Hz, 2H), 7.38-7.25 (m, 3H), 6.75 (s, 1H), 5.09 (s, 2H), 4.23 (s, 2H), 2.94 (br t, J=6.0 Hz, 2H), 2.82-2.58 (m, 3H), 2.11 (br dd, J=10.5, 16.6 Hz, 1H), 1.90-1.65 (m, 2H), 1.37 (s, 9H), 1.30-1.17 (m, 1H); ¹⁹F NMR (377 MHZ, DMSO-d₆) δ ppm −122.89 (br s, 1F); MS (ESI⁻) m/z 518 [M−H]⁻.

### Example 15J: 5-[7-(aminomethyl)-3-(benzyloxy)-1-fluoro-5,6,7,8-tetrahydronaphthalen-2-yl]-1λ⁶,2,5-thiadiazolidine-1,1,3-trione 2,2,2-trifluoroacetate

2,2,2-Trifluoroacetic acid (100 μL, 1.298 mmol) was added to a solution of the product of Example 151 (18.8 mg, 0.036 mmol) in dichloromethane (1 mL) and the mixture was stirred at ambient temperature for 1 hour and 15 minutes. The reaction mixture was concentrated under reduced pressure and the residue was azeotroped with toluene (3×2 mL). The title compound was used in the next reaction without further purification. MS (ESI⁺) m/z 420 [M+H]⁺.

### Example 15K: 5-[3-(benzyloxy)-1-fluoro-7-{[(2-methylpropyl)amino]methyl}-5,6,7,8-tetrahydronaphthalen-2-yl]-1λ⁶,2,5-thiadiazolidine-1,1,3-trione

Triethylamine (0.020 mL, 0.144 mmol) was added to the product of Example 15J in dichloromethane (1 mL) and ethanol (2 mL). The reaction mixture was stirred at ambient temperature for 5 minutes, after which isobutyraldehyde (0.017 mL, 0.180 mmol) was added and the mixture was stirred further for 2 hours. Sodium tetrahydroborate (10.90 mg, 0.288 mmol) was then added and the mixture was stirred for 30 minutes. The reaction mixture was quenched with aqueous 1 M HCl (0.5 mL) and concentrated under reduced pressure with diatomaceous earth for dry loading. The residue was purified by reverse phase column chromatography (30 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=25 mL/minute) to give the title compound (13.3 mg, 0.028 mmol, 78% yield). MS (ESI⁺) m/z 476 [M+H]⁺.

### Example 15L: 5-(1-fluoro-3-hydroxy-7-{[(2-methylpropyl)amino]methyl}-5,6,7,8-tetrahydronaphthalen-2-yl)-1λ⁶,2,5-thiadiazolidine-1,1,3-trione

The product of Example 15K (12.7 mg, 0.027 mmol), ammonium formate (13.7 mg, 0.217 mmol), and 10% Pd/C (2.84 mg, 2.67 μmol) in ethanol (2 mL) was heated to 50° C. for 1.5 hours. The reaction mixture was cooled to ambient temperature, filtered over diatomaceous earth, and rinsed with methanol. The filtrate was concentrated under reduced pressure and the residue was purified by reverse phase column chromatography (30 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=25 mL/minute) to give the title compound (3.2 mg, 8.30 μmol, 31.1% yield). ¹H NMR (400 MHZ, DMSO-d₆) δ ppm 8.99 (s, 1H), 6.44 (s, 1H), 3.93 (d, J=1.8 Hz, 2H), 2.90-2.81 (m, 3H), 2.73-2.66 (m, 4H), 2.20 (dd, J=15.7, 11.3 Hz, 1H), 2.02-1.86 (m, 3H), 1.37-1.32 (m, 1H), 0.94 (d, J=6.7 Hz, 6H); MS (APCI⁺) m/z 386 [M+H]⁺.

### Example 16:5-{1-fluoro-7-[(2-fluoro-3-methylbutyl)amino]-3-hydroxy-5,6,7,8-tetrahydronaphthalen-2-yl}-1λ⁶,2,5-thiadiazolidine-1,1,3-trione (Compound 334)

### Example 16A: 5-{3-(benzyloxy)-1-fluoro-7-[(2-fluoro-3-methylbutyl)amino]-5,6,7,8-tetrahydronaphthalen-2-yl}-1λ⁶,2,5-thiadiazolidine-1,1,3-trione

To a stirred mixture of 3-methylbutanal (70 μL, 0.610 mmol) and(S)-pyrrolidine-2-carboxylic acid (21 mg, 0.182 mmol) in acetonitrile (0.5 mL) was added 1-chloromethyl-4-fluoro-1,4-diazoniabicyclo[2,2,2]octane bis(tetrafluoroborate)) (318 mg, 0.898 mmol) in one portion at 0° C. After 2 minutes, trifluoroacetic acid (15 μL, 0.195 mmol) was added and the reaction mixture was stirred at 0° C., for 2.5 hours and then 30 minutes at room temperature. The crude mixture was passed through a short silica plug and the silica was washed with acetonitrile (2.3 mL). The filtrate (2.2 mL) was used as such immediately and added to a suspension of the product of Example 14A (97 mg, 0.191 mmol) and triethylamine (70 μL, 0.502 mmol) in N,N-dimethylformamide (1 mL). The reaction mixture was stirred for 30 minutes. After which time sodium triacetoxy borohydride (254 mg, 1.198 mmol) was added and the mixture was stirred for 16 hours. The reaction was quenched by the addition of solid ammonia hydrochloride (100 mg, 1.869 mmol) and water (0.7 mL) and the mixture was stirred for 5 minutes. The mixture was concentrated to dryness on diatomaceous earth. The suspension was subjected to column chromatography (Büchi Reveleris® C18, dry load with diatomaceous earth, 5-50% acetonitrile 0.1% formic acid in water 0.1% formic acid) to afford crude title compound. The crude material was further purified by reversed phase preparative HPLC on a Waters XSelect® CSH column C18, 5 μm, 30×100 mm, flow rate 42 mL/minute eluted with a 0.1% formic acid in water-acetonitrile gradient over 13 minutes (0.0-0.5 minute, 10% acetonitrile: 0.5-10.50 minutes, ramped from 10% acetonitrile to 40% acetonitrile: 10.5-10.6 minutes, ramped from 40% acetonitrile to 100% acetonitrile: 10.6-11.6 minutes, held at 100% acetonitrile: 11.6-11.7 minutes slowed from 100% acetonitrile to 10% acetonitrile and held for until 13 minutes) to afford the title compound (15 mg, 0.030 mmol, 13% yield). ¹H NMR (500 MHZ, methanol-d₄) δ ppm 7.54-7.50 (m, 2H), 7.37 (dd, J=8.4, 6.9 Hz, 2H), 7.33-7.26 (m, 1H), 6.70 (s, 1H), 5.15 (s, 2H), 4.48-4.32 (m, 1H), 4.28 (d, J=1.5 Hz, 2H), 3.10 (dt, J=16.2, 6.0 Hz, 1H), 2.99-2.76 (m, 4H), 2.69 (s, 1H), 2.37 (dd, J=16.2, 9.7 Hz, 1H), 2.14-2.05 (m, 1H), 1.97-1.84 (m, 1H), 1.58 (ddt, J=21.0, 10.6, 5.3 Hz, 1H), 1.04-0.96 (m, 6H); MS (ESI⁺) m/z 494 [M+H]⁺.

### Example 16B: 5-{1-fluoro-7-[(2-fluoro-3-methylbutyl)amino]-3-hydroxy-5,6,7,8-tetrahydronaphthalen-2-yl}-1λ⁶,2,5-thiadiazolidine-1,1,3-trione

The product of Example 16A (15 mg, 0.030 mmol) was dissolved in a mixture of degassed water (0.25 mL) and dioxane (0.25 mL). 10% Pd/C (5 mg) was added. The resulting suspension was allowed to stir under hydrogen (5 bar) for 2.5 hours. The mixture was diluted with 1,4-dioxane (1 mL) and water (1 mL) then filtered over a pad of diatomaceous earth. The pad was washed with methanol (3×1 mL). The combined filtrates were concentrated in vacuo to afford the title compound (12.7 mg, 0.030 mmol, 98% yield). ¹H NMR (500 MHZ, DMSO-d₆) δ ppm 9.15 (s, 1H), 6.52 (s, 1H), 6.45 (s, 1H), 4.66-4.38 (m, 1H), 3.93 (s, 2H), 3.30-3.11 (m, 3H), 3.05 (t, J=16.6 Hz, 1H), 2.84-2.63 (m, 2H), 2.50-2.41 (m, 1H), 2.20-2.02 (m, 1H), 1.99-1.87 (m, 1H), 1.70-1.57 (m, 1H), 0.98-0.89 (m, 6H); MS (ESI⁺) m/z 404 [M+H]⁺.

### Example 17:5-(1-fluoro-3,7-dihydroxy-5,6,7,8-tetrahydronaphthalen-2-yl)-1λ⁶,2,5-thiadiazolidine-1,1,3-trione (Compound 335)

### Example 17A: 5-[3-(benzyloxy)-1-fluoro-7-hydroxy-5,6,7,8-tetrahydronaphthalen-2-yl]-1λ⁶,2,5-thiadiazolidine-1,1,3-trione, ammonium salt

To a stirred mixture of Example 5F (649 mg, 1.605 mmol) and ammonium acetate (1237 mg, 16.05 mmol) in dioxane (6 mL) was added acetic acid (0.065 mL, 1.133 mmol). The mixture was stirred at room temperature for 30 minutes. Sodium triacetoxy borohydride (1364 mg, 6.44 mmol) was added and the reaction mixture was stirred for 16 hours. The reaction was cooled in an ice bath for 5 minutes then quenched with water (0.7 mL). The mixture was concentrated in vacuo. The crude residue was subjected to column chromatography (Büchi Reveleris® C18, dry load with diatomaceous earth, 5-50% acetonitrile in 10 mM ammonium bicarbonate) to afford impure title compound. The residue was precipitated from ethyl acetate:methanol (9:1). The mother liquors were combined and concentrated in vacuo. Half the material was further purified by reversed phase preparative HPLC on a Waters XSelect® CSH column C18, 5 μm 30×100 mm, flow rate 42 mL/minute, eluted with a 0.1% formic acid in water-acetonitrile gradient over 13 minutes (0.0-0.5 minute, 30% acetonitrile: 0.5-10.50 minutes, ramped from 30% acetonitrile to 60% acetonitrile: 10.5-10.6 minutes, ramped from 60% acetonitrile to 100% acetonitrile: 10.6-11.6 minutes, held at 100% acetonitrile: 11.6-11.7 minutes slowed from 100% acetonitrile to 30% acetonitrile and held for until 13 minutes) to afford the title compound as an ammonium salt (61.9 mg, 0.139 mmol, 9% yield). ¹H NMR (500 MHZ, DMSO-d₆) δ ppm 7.49-7.44 (m, 2H), 7.39-7.33 (m, 2H), 7.33-7.27 (m, 1H), 7.24-6.91 (m, 1H), 6.76 (s, 1H), 5.11 (s, 2H), 4.23 (s, 2H), 3.99-3.90 (m, 1H), 2.89-2.77 (m, 2H), 2.65-2.73 (m, 1H), 2.42 (dd, J=16.5, 6.8 Hz, 1H), 1.85-1.79 (m, 1H), 1.71-1.59 (m, 1H); MS (ESI⁺) m/z 405 [M−H]⁻.

### Example 17B: 5-(1-fluoro-3,7-dihydroxy-5,6,7,8-tetrahydronaphthalen-2-yl)-1λ⁶,2,5-thiadiazolidine-1,1,3-trione

The product of Example 17A (62 mg, 0.153 mmol) was dissolved in a mixture of water (0.25 mL) and dioxane (0.25 mL). 10% Pd/C (20 mg) was added, and the reaction mixture was submitted to hydrogenation for 3 hours at 5 bar. Additional 10% Pd/C (7 mg) was added, and the reaction mixture was resubmitted to 5 bar hydrogenation for a further 1.5 hours. The mixture was filtered and the solids were washed with dioxane (1 mL) and water (1 mL) then methanol (2×1 mL). The combined washes were concentrated in vacuo and dried in a desiccator at 45° C. for 16 hours to afford the title compound as an ammonium salt (40.7 mg, 0.116 mmol, 76% yield). ¹H NMR (500 MHZ, DMSO-d₆) δ ppm 9.50 (s, 1H), 7.28-6.92 (m, 1H), 6.44 (s, 1H), 4.15 (s, 2H), 3.94-3.86 (m, 1H), 2.81-2.73 (m, 2H), 2.67-2.57 (m, 1H), 2.36 (dd, J=16.3, 7.2 Hz, 1H), 1.83-1.77 (m, 1H), 1.66-1.55 (m, 1H); MS (ESI⁻) m/z 315 [M−H]⁻.

### Example 18: 5-{7-[(²H₉)butylamino]-1-fluoro-3-hydroxy-5,6,7,8-tetrahydronaphthalen-2-yl}-1λ⁶,2,5-thiadiazolidine-1,1,3-trione (Compound 336)

### Example 18A: 5-{3-(benzyloxy)-7-[(²H₉)butylamino]-1-fluoro-5,6,7,8-tetrahydronaphthalen-2-yl}-1λ⁶,2,5-thiadiazolidine-1,1,3-trione

(²H₉)Butan-1-amine (0.061 mL, 0.558 mmol) was added to a solution of the product of Example 5F (150.4 mg, 0.372 mmol) in ethanol (2 mL) and the mixture was stirred for 1 hour at ambient temperature. Then sodium cyanoborohydride (28.0 mg, 0.446 mmol) was added and the mixture was stirred further at ambient temperature for 23 hours. The reaction mixture was diluted with methanol (10 mL) and concentrated with diatomaceous earth for dry loading. The material was purified by reverse phase flash column chromatography (60 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=50 mL/minute) to afford the title compound (73.6 mg, 0.156 mmol, 42.1% yield). ¹H NMR (500 MHZ, DMSO-d₆) δ ppm 8.42 (s, 2H), 7.54-7.48 (m, 2H), 7.40-7.34 (m, 2H), 7.34-7.28 (m, 1H), 6.76 (s, 1H), 5.13 (s, 2H), 4.01-3.92 (m, 2H), 3.46 (s, 1H), 3.14 (dd, J=16.2, 5.5 Hz, 1H), 2.91-2.74 (m, 2H), 2.58 (dd, J=16.2, 9.8 Hz, 1H), 2.18 (s, 1H), 1.72 (qd, J=11.4, 5.6 Hz, 1H); MS (APCI+) m/z 471 [M+H]⁺.

### Example 18B: 5-{3-(benzyloxy)-7-[(²H₉)butylamino]-1-fluoro-5,6,7,8-tetrahydronaphthalen-2-yl}-1λ⁶,2,5-thiadiazolidine-1,1,3-trione

The product of Example 18A (72.3 mg, 0.154 mmol) in tetrahydrofuran (2 mL) was added to 5% Pd/C (140 mg, 0.613 mmol) in a 20 mL Barnstead STEM RS10 reactor and the mixture was stirred at 25° C. under an atmosphere of hydrogen at 63-98 psi for 19.7 hours. The reaction mixture was filtered, and the filtrate was concentrated under reduced pressure with diatomaceous earth for dry loading. The material was purified by reverse phase column chromatography (60 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=50 mL/minute) to give the title compound as the ammonium salt. ¹H NMR (400 MHz, DMSO-d₆) δ ppm 9.20 (s, 1H), 8.32 (s, 3H), 6.47 (s, 1H), 3.93 (s, 2H), 3.17 (d, J=4.4 Hz, 1H), 3.12-3.03 (m, 1H), 2.85-2.66 (m, 3H), 2.15-2.11 (m, 1H), 1.69-1.64 (m, 1H); MS (ESI⁺) m/z 381 [M+H]⁺.

### Example 19:5-[7-(aminomethyl)-1-fluoro-3,7-dihydroxy-5,6,7,8-tetrahydronaphthalen-2-yl]-1λ⁶,2,5-thiadiazolidine-1,1,3-trione (Compound 337)

### Example 19A: 5-[3-(benzyloxy)-1-fluoro-7-hydroxy-7-(nitromethyl)-5,6,7,8-tetrahydronaphthalen-2-yl]-1λ⁶,2,5-thiadiazolidine-1,1,3-trione

To a solution of the product of Example 5F (553 mg, 1.231 mmol) and nitromethane (1.5 mL, 27.8 mmol) in N-methyl-2-pyrrolidinone (0.25 mL) at 0° C. was added potassium 2-methylpropan-2-olate (2 M solution in tetrahydrofuran) (1.570) mL, 3.14 mmol) in N-methyl-2-pyrrolidinone (0.25 mL). The reaction mixture was stirred for 30 minutes, after which time the ice bath was removed and the mixture was stirred for 16 hours. The reaction was quenched with a mixture 1/1 v/v of acetic acid and acetonitrile. The mixture was concentrated in vacuo. The crude residue was subjected to column chromatography (Büchi Reveleris® C18, 5-80% acetonitrile in 0.1% aqueous formic acid) to afford the title compound (404 mg, 0.764 mmol, 62% yield, 88% purity). ¹H NMR (500 MHZ, methanol-d₄) δ ppm 7.51-7.45 (m, 2H), 7.41-7.34 (m, 2H), 7.36-7.29 (m, 1H), 6.84-6.76 (m, 1H), 5.14 (d, J=5.3 Hz, 2H), 4.68-4.60 (m, 2H), 4.41 (s, 2H), 3.06 (ddd, J=16.8, 10.3, 5.9 Hz, 1H), 2.91-2.82 (m, 3H), 2.08-1.97 (m, 1H), 1.88 (ddd, J=13.4, 10.4, 5.8 Hz, 1H); MS (ESI⁻) m/z 464 [M−H]⁻.

### Example 19B: 5-[7-(aminomethyl)-3-(benzyloxy)-1-fluoro-7-hydroxy-5,6,7,8-tetrahydronaphthalen-2-yl]-1λ⁶,2,5-thiadiazolidine-1,1,3-trione

To an ice-cold suspension of the product of Example 19A (395 mg, 0.747 mmol) and nickel chloride hexahydrate (236 mg, 0.993 mmol) in methanol (5 mL) was added sodium borohydride (198 mg, 5.23 mmol) in small portions. The ice bath was removed, and the reaction mixture was stirred for 30 minutes. The mixture was then quenched with a saturated aqueous solution of ammonium chloride (1.5 mL) and stirred for another 30 minutes at room temperature. Diatomaceous earth (2 g) was added, and the mixture was concentrated in vacuo. The crude residue was subjected to column chromatography (Büchi Reveleris® C18, 0-100% methanol in 10 mM ammonium hydroxide) to afford the title compound (261 mg, 0.569 mmol, 76% yield). ¹H NMR (500 MHZ, DMSO-d₆) δ ppm 7.77 (s, 3H), 7.53-7.47 (m, 2H), 7.38-7.32 (m, 2H), 7.32-7.26 (m, 1H), 6.73 (s, 1H), 5.19 (s, 1H), 5.11 (s, 2H), 4.00-3.90 (m, 2H), 2.95-2.80 (m, 3H), 2.60-2.73 (m, 3H), 1.81 (dt, J=11.2, 5.2 Hz, 1H), 1.63 (ddd, J=12.9, 10.3, 5.6 Hz, 1H); MS (ESI⁺) m/z 436 [M+H]⁺.

### Example 19C: 5-[7-(aminomethyl)-1-fluoro-3,7-dihydroxy-5,6,7,8-tetrahydronaphthalen-2-yl]-1λ⁶,2,5-thiadiazolidine-1,1,3-trione

The product of Example 19B (50 mg, 0.109 mmol) was dissolved in a mixture of water (1.5 mL) and dioxane (1.5 mL). 10% Pd/C (12 mg) was added. The resulting suspension was allowed to stir under hydrogen (5 bar) for 4 hours. The mixture was diluted with 0.05:1:1 v/v/v formic acid:dioxane:water (5 mL) and stirred for 10 minutes. The suspension was filtered over a pad of diatomaceous earth. The filter was washed with hot methanol (2×5 mL). The combined organic filtrates were concentrated in vacuo. The residual solid was stirred for 16 hours in acetonitrile (3 mL). The suspension was separated by centrifuge. The diatomaceous earth filter was further washed with a hot methanolic ammonium hydroxide solution (1.3% v/v, 150 mL at about 60° C.). The previous solid and methanolic solution were combined and concentrated in vacuo to afford the title compound (35 mg, 0.097 mmol, 88% yield). ¹H NMR (400 MHz, DMSO-d₆) δ ppm 8.97-7.73 (m, 4H), 6.47 (s, 1H), 5.16 (s, 1H), 3.94 (s, 2H), 2.92-2.78 (m, 3H), 2.65 (dd, J=17.8, 7.0 Hz, 3H), 1.88-1.58 (m, 2H); MS (ESI⁺) m/z 346 [M+H]⁺.

### Example 20: 5-[(7R)-1-fluoro-3-hydroxy-7-({2-[1-(hydroxymethyl)cyclobutyl]ethyl}amino)-5,6,7,8-tetrahydronaphthalen-2-yl]-1λ⁶,2,5-thiadiazolidine-1,1,3-trione (Compound 338)

### Example 20A: tert-butyl(dimethyl){[1-(prop-2-en-1-yl)cyclobutyl]methoxy}silane

To a solution of (1-allylcyclobutyl)methanol (prepared according to Bioorganic and Medicinal Chemistry, 2002, 10(4), 1093-1106) (2.5 g, 15.85 mmol, purity 80%) in anhydrous tetrahydrofuran (70) mL) was added imidazole (2.158 g, 31.7 mmol) and then tert-butyldimethylchlorosilane (3.58 g, 23.77 mmol) was added in portions at 0° C. The reaction mixture was stirred at 20° C. for 3 hours. One additional reaction on 500 mg scale was set up as described above. These two reaction mixtures were combined and diluted with water (200 mL), the organic phase was separated, and the aqueous phase was extracted with ethyl acetate (60) mL). The combined organic phases were washed with brine (100 mL), dried over anhydrous sodium sulfate, and filtered, and the filtrate was concentrated under reduced pressure. The residue was then dissolved with petroleum ether and filtered through silica gel, and the filter cake was washed with petroleum ether (1500 mL). The filtrate was concentrated under reduced pressure to give the title compound (4 g, yield 86%). ¹H NMR (400 MHZ, CDCl₃) δ ppm 5.79 (ddt, J=17.07, 10.07, 7.32 Hz, 1H), 4.96-5.10 (m, 2H), 3.44 (s, 2H), 2.21 (d, J=7.25 Hz, 2H), 1.63-1.92 (m, 6H), 0.88-0.95 (m, 9H), −0.01-0.12 (m, 6H).

### Example 20B: [1-({[tert-butyl(dimethyl)silyl]oxy}methyl)cyclobutyl]acetaldehyde

To a solution of the product of Example 20A (3 g, 11.23 mmol, purity 90%) in dioxane (120 mL) and water (12 mL) was added a 0.2 M solution osmium tetroxide in t-butanol (220 mg, 0.865 mmol)) dropwise at 20° C. After 15 minutes, the reaction mixture was cooled to 0° C. before sodium periodate (9.61 g, 44.9 mmol) was added in portions. After the addition, the mixture was warmed up to 20° C. and stirred at that temperature for 3 hours. The mixture was diluted with ethyl acetate (200 mL) and filtered. The filtrate was added to saturated sodium thiosulfate aqueous solution (300 mL) and the resulting mixture was stirred at 20° C. for 1 hour. The mixture was transferred to a separatory funnel and the organic phase was separated, washed with brine (500 mL), dried over anhydrous sodium sulfate and filtered, and the filtrate was concentrated under reduced pressure to give the title compound (3 g, purity 70%, yield 77%) which was used for the next step without further purification. ¹H NMR (400 MHZ, CDCl₃) δ ppm 9.75 (t, J=2.75 Hz, 1H), 3.61 (s, 2H), 2.50 (d, J=2.63 Hz, 2H), 1.82-2.00 (m, 9H), 0.89 (s, 11H), 0.03-0.06 (m, 7H).

### Example 20C; tert-butyl [(2R)-6-(benzyloxy)-7-bromo-8-fluoro-1,2,3,4-tetrahydronaphthalen-2-yl]carbamate

To a solution of Example 6B (5 g, 12.85 mmol, purity 90%) in tetrahydrofuran (30 mL) was added a solution of sodium bicarbonate (2.159 g, 25.7 mmol) in water (30 mL) at 20° C. followed by di-tert-butyl dicarbonate (3.58 mL, 15.42 mmol). The mixture was stirred at 20° C. for 2 hours. Three additional reactions on 5 g scale were run as described above. The reaction mixtures were combined and diluted with water (50 mL). The mixture was extracted with ethyl acetate (3× 200 mL). The combined organic layers were washed with brine (100 mL), dried over anhydrous sodium sulfate and concentrated under reduced pressure. The residue was purified by column chromatography on silica gel eluted with tetrahydrofuran-petroleum ether (10%-40%) to give the title compound (50 g, 110 mmol, purity 99%, yield 86%), MS (ESI⁻) m/z 350 [M-99].

### Example 20D; tert-butyl ({(7R)-3-(benzyloxy)-7-[(tert-butoxycarbonyl)amino]-1-fluoro-5,6,7,8-tetrahydronaphthalen-2-yl}amino)acetate

The title compound was prepared in (93% yield) from the product of Example 20C by the same procedure as described for Example 6D. MS (ESI⁺) m/z 501 [M+H]⁺

### Example 20E: tert-butyl [{(7R)-3-(benzyloxy)-7-[(tert-butoxycarbonyl)amino]-1-fluoro-5,6,7,8-tetrahydronaphthalen-2-yl}({[(prop-2-en-1-yl)oxy]carbonyl}sulfamoyl)amino]acetate

The title compound was prepared in (50% yield) from the product of Example 20D by the same procedure as described for Example 6E. MS (ESI⁺) m/z 686 [M+Na]⁺

### Example 20F: tert-butyl [(2R)-6-(benzyloxy)-8-fluoro-7-(1,1,4-trioxo-1λ⁶,2,5-thiadiazolidin-2-yl)-1,2,3,4-tetrahydronaphthalen-2-yl]carbamate

The title compound was prepared in 62% yield from the product of Example 20E by the same procedure as described for Example 6F. MS (ESI⁻) m/z 504 [M−H]⁻

### Example 20G: 5-[(7R)-7-amino-3-(benzyloxy)-1-fluoro-5,6,7,8-tetrahydronaphthalen-2-yl]-1λ⁶,2,5-thiadiazolidine-1,1,3-trione

To solution of Example 20F (11.2 g, 21.05 mmol) in ethyl acetate (150 mL) was added a solution of hydrogen chloride in ethyl acetate (150 mL, 4 mol/L) dropwise at 0° C. The mixture was then stirred at 20° C. for 12 hours resulting in a suspension. One additional reaction on 1 g scale was run as described above. The reaction mixtures were combined and the solids were collected by filtration and dried in an oven at room temperature under vacuum to give the title compound as a hydrochloric acid salt (10.27 g, 92% pure, 93% yield). ¹H NMR (400 MHZ, DMSO-d₆) δ ppm 8.41 (br s, 3H), 7.45 (d, J=7.00 Hz, 2H), 7.27-7.40 (m, 3H), 6.86 (s, 1H), 5.14 (s, 2H), 4.37 (s, 2H), 3.43 (br s, 1H), 3.07 (br dd, J=16.20, 5.19 Hz, 1H), 2.75-2.91 (m, 2H), 2.61 (br dd, J=16.26, 9.88 Hz, 1H), 2.06-2.19 (m, 1H), 1.77 (qd, J=11.32, 6.19 Hz, 1H); MS (ESI⁻) m/z 404 [M−H]⁻.

### Example 20H: 5-[(7R)-3-(benzyloxy)-7-({2-[1-({[tert-butyl(dimethyl)silyl]oxy}methyl)cyclobutyl]ethyl}amino)-1-fluoro-5,6,7,8-tetrahydronaphthalen-2-yl]-1λ⁶,2,5-thiadiazolidine-1,1,3-trione

To a mixture of the product of Example 20G hydrochloric acid salt (400 mg, 0.815 mmol, purity 90%) in dichloromethane (12 mL) and ethanol (8 mL) was added triethylamine (247 mg, 2.444 mmol) and the mixture was stirred for 5 minutes at 20° C. Then the product of Example 20B (1.2 g, 3.46 mmol) was added dropwise neat at 20° C. The mixture was stirred at 20° C. for 2 hours. The mixture was cooled to 0° C. and NaBH₄ (154 mg, 4.07 mmol) was then added in portions. The mixture was allowed to warm up to 20° C. and was stirred for 20 minutes. Then the mixture was quenched with methanol (1 mL) dropwise at 0° C. The mixture was stirred for 20 minutes after the quench, and then was diluted with water (20 mL). The resulting mixture was filtered and the filtrate was extracted with dichloromethane (2×15 mL). The combined organic phases were dried over anhydrous sodium sulfate and filtered, and the filtrate was concentrated under reduced pressure. The residue was triturated with petroleum ether (2 mL) to give the title compound (470 mg, 90% purity, 82% yield). ¹H NMR (400 MHZ, DMSO-d₆) δ ppm 8.28-8.68 (m, 1H), 7.49 (br d, J=7.45 Hz, 2H), 7.26-7.39 (m, 3H), 5.12 (s, 2H), 6.75 (s, 1H), 3.89-4.01 (m, 2H), 3.51 (s, 2H), 3.12 (br dd, J=16.55, 4.49 Hz, 1H), 2.93-3.03 (m, 2H), 2.76-2.89 (m, 2H), 2.53-2.61 (m, 1H), 2.11-2.23 (m, 1H), 1.66-1.88 (m, 12H), 0.90 (s, 10H), 0.07 (s, 6H).

### Example 20I: 5-[(7R)-1-fluoro-3-hydroxy-7-({2-[1-(hydroxymethyl)cyclobutyl]ethyl}amino)-5,6,7,8-tetrahydronaphthalen-2-yl]-1λ⁶,2,5-thiadiazolidine-1,1,3-trione

To a mixture of the product of Example 20H (370 mg, 0.527 mmol, purity 90%) in methanol (15 mL) and hydrochloric acid (3 mL, 1 mol/L aqueous solution) was added 10% Pd/C (56.1 mg, 0.527 mmol) and the mixture was stirred under H₂ (15 psi) at 20° C. for 2 hours. One additional reaction on 100 mg scale was run as described above. These two reaction mixtures were combined and filtered, and the filtrate was adjusted to pH=7 with NaHCO₃ (solid). The resulting mixture was concentrated under reduced pressure. The residue was purified by preparative HPLC on Welch Xtimate® C18 column (100×25 mm, 3 μm) eluted with 5-30% acetonitrile in water with 0.04% HCl at flow rate of 25 mL/minute to give the title compound as hydrochloric acid salt (173 mg, yield 55%). ¹H NMR (400 MHz, DMSO-d₆) δ ppm 9.84 (br s. 1H), 8.73 (br s, 2H), 6.50 (s, 1H), 4.18 (s, 2H), 3.36 (s, 3H), 3.11 (br dd, J=15.88, 4.75 Hz, 1H), 2.92-3.02 (m, 2H), 2.65-2.87 (m, 2H), 2.56 (br dd, J=16.26, 10.51 Hz, 1H), 2.18 (br d, J=10.88 Hz, 1H), 1.61-1.88 (m, 10H). ¹H NMR (400 MHz, DMSO&D₂O) δ ppm 6.51 (s, 1H), 4.17 (s, 2H), 3.41 (br d, J=7.63 Hz, 1H), 3.35 (s, 2H), 3.10 (br dd, J=15.63, 4.88 Hz, 1H), 2.91-3.01 (m, 2H), 2.65-2.86 (m, 2H), 2.55 (br d, J=10.38 Hz, 1H), 2.17 (br d, J=10.38 Hz, 1H), 1.60-1.87 (m, 10H); MS (ESI⁻) m/z 426 [M−H]⁻.

### Example 21: 5-(1-fluoro-3,7-dihydroxy-7-{[(2-methylpropyl)amino]methyl}-5,6,7,8-tetrahydronaphthalen-2-yl)-1λ⁶,2,5-thiadiazolidine-1,1,3-trione (Compound 339)

### Example 21A: 5-[3-(benzyloxy)-1-fluoro-7-hydroxy-7-{[(2-methylpropyl)amino]methyl}-5,6,7,8-tetrahydronaphthalen-2-yl]-1λ⁶,2,5-thiadiazolidine-1,1,3-trione, ammonium salt

Isobutyraldehyde (37 mg, 0.513 mmol) was added to a suspension of the product of Example 19B (202 mg, 0.464 mmol) in N,N-dimethylformamide (1 mL) and the mixture was stirred for 30 minutes. Sodium triacetoxy borohydride (236 mg, 1.113 mmol) was added and the mixture was stirred for 16 hours. The reaction was quenched with a saturated aqueous solution of ammonium chloride (0.5 mL) and water (0.5 mL). Diatomaceous earth was added, and the mixture was concentrated in vacuo. The crude residue was subjected to column chromatography (Büchi Reveleris® C18, dry load with diatomaceous earth, 0-100% methanol in 10 mM ammonium hydroxide) to afford the title compound as an ammonium salt (88 mg, 0.164 mmol. 35% yield). ¹H NMR (400 MHZ, DMSO-d₆) δ ppm 7.53-7.47 (m, 2H), 7.45 (s, 5H), 7.39-7.25 (m, 3H), 6.73 (s, 1H), 5.32 (bs, 1H), 5.11 (s, 2H), 3.95 (d, J=1.9 Hz, 2H), 3.08-2.93 (m, 2H), 2.92-2.64 (m, 6H), 2.05 (dt, J=13.6, 6.8 Hz, 1H), 1.90-1.82 (m, 1H), 1.74-1.64 (m, 1H), 0.95 (d, J=6.7 Hz, 6H); MS (ESI⁺) m/z 492 [M+H]⁺.

### Example 21B: 5-(1-fluoro-3,7-dihydroxy-7-{[(2-methylpropyl)amino]methyl}-5,6,7,8-tetrahydronaphthalen-2-yl)-1λ⁶,2,5-thiadiazolidine-1,1,3-trione

The product of Example 21A (50 mg, 0.098 mmol) was suspended in a mixture of water (2 mL) and dioxane (2 mL). A saturated aqueous solution of sodium carbonate (0.4 mL, 0.832 mmol) was added to obtain a solution. 10% Pd/C (12 mg) was added, and the resulting suspension was allowed to stir under hydrogen (5 bar) for 4 hours. The mixture was diluted in dioxane (2 mL) and filtered over a pad of diatomaceous earth, then washed with water (2 mL) and a mixture of concentrated ammonium hydroxide (2 mL, 27%) and methanol (150 mL). The filtrate was concentrated in vacuo. The residue was resuspended in water (2 mL) then hydrogen chloride (1 M aqueous solution) was added until it reached pH ˜6-7. The suspension was centrifuged, the pellet was resuspended and was separated by centrifugation (cycle repeated 3 times, with 1 mL of water each time). The solid was then suspended in a minimum amount of water and azeotroped with acetonitrile (2×1 mL) to afford the title compound (37 mg, 0.090 mmol, 92% yield). ¹H NMR (400 MHz, DMSO-d₆) δ ppm 9.08 (s, 1H), 8.11 (s, 2H), 6.47 (s, 1H), 5.27 (s, 1H), 3.94 (s, 2H), 3.03 (d, J=12.7 Hz, 1H), 2.95 (d, J=12.7 Hz, 1H), 2.90-2.72 (m, 3H), 2.67 (t, J=12.7 Hz, 3H), 2.11-1.98 (m, 1H), 1.87-1.79 (m, 1H), 1.73-1.61 (m, 1H), 0.95 (d, J=6.6 Hz, 6H); MS (ESI⁺) m/z 402 [M+H]⁺.

### Example 22: 5-{1-fluoro-3-hydroxy-7-[(3-methylbutyl)amino](6,6,7,8,8-²H₅)-5,6,7,8-tetrahydronaphthalen-2-yl}-1λ⁶,2,5-thiadiazolidine-1,1,3-trione (Compound 340)

### Example 22A: 5-[3-(benzyloxy)-1-fluoro-7-oxo (6,6,8,8-²H₄)-5,6,7,8-tetrahydronaphthalen-2-yl]-1λ⁶,2,5-thiadiazolidine-1,1,3-trione

A solution of dioxane (0.25 mL) containing pyrrolidine (5.0 μL, 0.060 mmol), the product of Example 5F (242 mg, 0.598 mmol), and D₂O (0.250 mL) was stirred at ambient temperature while N₂ was sparged via subsurface bubbling for 5 minutes. The mixture was heated in a 60° C. heating block for 60 hours, then was cooled to ambient temperature and formic acid (0.011 mL, 0.299 mmol) was added. After diluting with methyl tert-butyl ether (MTBE) (10 mL) and water (5 mL), the layers were separated. The organic layer was washed with brine (2 mL), dried (Na₂SO₄), and filtered, and the filtrate was concentrated under reduced pressure to minimal volume. Formic acid (2.4 mL) and water (1.2 mL) were added and a slurry developed. The slurry was stirred for 10 minutes and filtered, washing with water (3×2 mL). The solid was dried in a vacuum oven at 50° C. to constant weight, giving the title compound (136 mg, 0.333 mmol, 56% yield). ¹H NMR (400 MHZ, CDCl₃) δ ppm 7.45-7.33 (m, 5H), 6.74 (s, 1H), 5.15 (s, 2H), 4.39 (s, 2H), 3.06 (s, 2H), 1.26 (s, 1H); MS (APCI⁻) m/z 407 [M−H]⁻.

### Example 22B: 5-[3-(benzyloxy)-1-fluoro-7-[(3-methylbutyl)amino](6,6,7,8,8-²H₅)-5,6,7,8-tetrahydronaphthalen-2-yl]-1λ⁶,2,5-thiadiazolidine-1,1,3-trione

A slurry of the product of Example 22A (132 mg, 0.323 mmol) and ethanol-d₆ (1 mL) was stirred at ambient temperature while N₂ was sparged via subsurface bubbling for 5 minutes, then isoamylamine (0.056 mL, 0.485 mmol) was added. After N₂ was sparged via subsurface bubbling for 5 additional minutes, the mixture was stirred for 20 minutes, then sodium cyanoborodeuteride (25.5 mg, 0.388 mmol) was added. After 19 hours, concentrated aqueous ammonium hydroxide (0.129 mL, 1.94 mmol) was added, the mixture was concentrated under reduced pressure, and the reaction mixture was directly purified by preparative HPLC [YMC TriArt™ C18 Hybrid 5 μm column, 50×100 mm, flow rate 120 mL/minute, 3-100% gradient of methanol in buffer (0.025 M aqueous ammonium bicarbonate, adjusted to pH 10 with ammonium hydroxide)] to give the title compound (52 mg, 0.11 mmol, 34% yield). ¹H NMR (600 MHZ, DMSO-d₆/D₂O) δ ppm 7.55-7.50 (m, 2H), 7.43-7.32 (m, 3H), 6.80 (s, 1H), 5.15 (s, 2H), 4.05 (d, J=1.4 Hz, 2H), 3.03 (td, J=7.6, 4.6 Hz, 2H), 2.82 (s, 2H), 1.53 (q, J=7.8 Hz, 2H), 0.95 (d, J=6.6 Hz, 6H). (400 MHZ, CDCl₃) δ ppm 7.45-7.33 (m, 5H), 6.74 (s, 1H), 5.15 (s, 2H), 4.39 (s, 2H), 3.06 (s, 2H), 1.74-1.64 (m, 1H), 1.26 (s, 1H); MS (APCI⁻) m/z 479 [M−H]⁻

### Example 22C: 5-{1-fluoro-3-hydroxy-7-[(3-methylbutyl)amino](6,6,7,8,8-²H₅)-5,6,7,8-tetrahydronaphthalen-2-yl}-1λ⁶,2,5-thiadiazolidine-1,1,3-trione

A mixture of the product of Example 22B (50.0 mg, 0.104 mmol), tetrahydrofuran (2 mL), and D₂O (0.4 mL) was added to 5% Pd/C (wet) (100 mg, 0.438 mmol) in a 20 mL Barnstead reactor with a glass liner and the mixture was stirred under D₂ (102 psi) at 25° C. After 2 hours, the mixture was filtered and the catalyst residue was washed with tetrahydrofuran and 0.1 M aqueous NaOH. After concentration, the crude product was dissolved in methanol (2 mL), filtered through a glass microfiber frit, and purified by preparative HPLC [YMC TriArt™ C18 Hybrid 5 μm column, 20×150 mm, flow rate 22 mL/minute, 0-100% gradient (3%-3% over 4 minutes, then 3%-50% over 22 minutes, wash at 100% for 6 minutes) of methanol in buffer (0.025 M aqueous ammonium bicarbonate)]. Product-containing fractions were concentrated to minimal volume, and the residue was suspended in acetonitrile (1 mL). The slurry was sonicated for 1 minute and filtered, washing with acetonitrile (2×0.2 mL). The solid was dried in a vacuum oven at 50° C. to constant weight, giving the title compound (30 mg, 0.077 mmol, 74% yield). ¹H NMR (500 MHZ, DMSO-d₆) δ ppm 9.27 (brs, 1H), 6.48 (s, 1H), 3.96 (s, 2H), 3.05-2.99 (m, 2H), 2.84-2.69 (m, 2H), 1.67 (dq, J=13.2, 6.6 Hz, 1H), 1.51 (q, J=7.3 Hz, 2H), 0.93 (d, J=6.5 Hz, 6H); MS (ESI⁻) m/z 389 [M−H]⁻.

### Example 23: tert-butyl [(2R)-8-fluoro-6-hydroxy-7-(1,1,4-trioxo-1λ⁶,2,5-thiadiazolidin-2-yl)-1,2,3,4-tetrahydronaphthalen-2-yl]carbamate (Compound 341)

### Example 23A: 5-[(7R)-7-amino-1-fluoro-3-hydroxy-5,6,7,8-tetrahydronaphthalen-2-yl]-1λ⁶,2,5-thiadiazolidine-1,1,3-trione

To a solution of the product of Example 6F (2 g, 3.61 mmol, 97.5% pure) in methanol (100 mL) was added aqueous hydrochloric acid (1 mol/L) (10.84 g, 10.84 mmol). 20% palladium hydroxide on carbon (0.254 g, 0.361 mmol) and 10% Pd/C (0.385 g, 0.361 mmol) under N₂ at 20° C. Then the mixture was stirred under H₂ (15 psi) at 20° C. for 2 hours. Two additional reactions on 2 g scale and one reaction on 1 g scale were run as described above. These four reaction mixtures were combined and filtered. The filtrate was adjusted to pH=9 with triethylamine and was concentrated under reduced pressure. The residue was triturated with a mixture of water and tetrahydrofuran (5:1), and the solid was collected by filtration and dried under high vacuum to give the title compound (1.9 g, 6.03 mmol, yield 47.6%). ¹H NMR (400 MHz, DMSO-d₆) δ ppm 9.24 (s, 1H), 7.96 (br s. 3H), 6.46 (s, 1H), 3.93 (s, 2H), 3.49-3.39 (m, 1H), 3.00 (br dd, J=5.2, 16.1 Hz, 1H), 2.82-2.71 (m, 2H), 2.45 (br d, J=9.8 Hz, 1H), 2.08-1.98 (m, 1H), 1.75-1.60 (m, 1H); MS (ESI⁻) m/z 314 [M−H]⁻.

### Example 23B: tert-butyl [(2R)-8-fluoro-6-hydroxy-7-(1,1,4-trioxo-1λ⁶,2,5-thiadiazolidin-2-yl)-1,2,3,4-tetrahydronaphthalen-2-yl]carbamate

To a solution of the product of Example 23A (1 g, 3.17 mmol) in water (300 mL) and tetrahydrofuran (50) mL) was added sodium bicarbonate (0.533 g, 6.34 mmol) followed by di-tert-butyl dicarbonate (0.884 mL, 3.81 mmol) at 20° C. Then the mixture was stirred at 20° C. for 12 hours. The solvent was removed under vacuum and the residue was diluted with water and lyophilized. The lyophilized material was purified by preparative HPLC on Kromasil® C18 (250×50 mm, 10 μm) column eluted with acetonitrile in water with aqueous 10 mM NH₄HCO₃ (5% to 35% in 20 minutes) at a flow rate of 80 mL/minute to give the title compound (1.02 g, yield 73.4%). ¹H NMR (400 MHz, DMSO-d₆) δ ppm 9.05 (br s, 1H), 7.09 (br d, 3H, J=3.5 Hz), 6.95 (br d, 1H, J=6.8 Hz), 6.41 (s, 1H), 3.92 (d, 2H, J=1.1 Hz), 3.57 (br s, 1H), 2.80 (br dd, 1H, J=4.6, 16.1 Hz), 2.6-2.7 (m, 2H), 2.32 (br dd, 1H, J=9.7, 16.3 Hz), 1.8-1.9 (m, 1H), 1.4-1.6 (m, 1H), 1.40 (s, 9H); MS (ESI⁻) m/z 414 [M−H]⁻.

### Example 24: 5-[(7R)-1-fluoro-3-hydroxy-7-{[(thiophen-3-yl)methyl]amino}-5,6,7,8-tetrahydronaphthalen-2-yl]-1λ⁶,2,5-thiadiazolidine-1,1,3-trione (Compound 342)

The product of Example 23A (25 mg, 0.08 mmol, 1.0 equivalent) was dissolved in 1.5 mL of acetic acid/sodium acetate buffer in methanol (adjusted to pH=4.5), and the mixture was added to a 4 mL vial containing 3-thiophenecarboxaldehyde (0.1 mmol, 1.2 equivalents). The vial was sealed with a PTFE cap and shaken for 1 hour at room temperature. To the vessel was added MP-CNBH₃ resin (108 mg, 3 equivalents. 2.19 mmol/g loading), and the suspension was shaken overnight at room temperature. The reaction mixture was filtered, and the filtrate was purified using HPLC on two coupled Phenomenex® Luna® C8(2) 5 μm 100 Å AXIA™ columns (30) mm×75 mm each). A gradient of acetonitrile (A) and 10 mM ammonium acetate in water (B) was used, at a flow rate of 50 mL/minute (0-0.5 minutes 5% A, 0.5-10.5 minutes linear gradient 5-100% A, 10.51-13.6 100% A 60 mL/minute. 13.6-14.0 minutes linear gradient 100-5% A 50 mL/minute) to afford the title compound (0.7 mg, 1.9% yield). ¹H NMR (400 MHz, DMSO-d₆) δ ppm 7.52-7.41 (m, 2H), 7.14-7.08 (m, 1H), 6.40 (s, 1H), 3.92 (s, 2H), 3.79 (s, 2H), 2.61 (d, J=52.1 Hz, 5H), 1.17-1.12 (m, 1H), 1.08-1.03 (m, 1H); MS (APCI+) m/z 412.1 [M+H]⁺.

### Example 25: 5-[(7R)-1-fluoro-3-hydroxy-7-{[(thiophen-2-yl)methyl]amino}-5,6,7,8-tetrahydronaphthalen-2-yl]-1λ⁶,2,5-thiadiazolidine-1,1,3-trione (Compound 343)

The product of Example 25 was prepared using the general procedure described in Example 24, substituting 2-thiophenecarboxaldehyde for 3-thiophenecarboxaldehyde (0.7 mg, 1.9% yield). ¹H NMR (400 MHZ, DMSO-d₆) δ ppm 9.46-8.79 (m, 1H), 7.77-6.83 (m, 3H), 6.45 (s, 1H), 4.74-4.13 (m, 2H), 3.93 (s, 2H), 3.18-2.62 (m, 5H), 2.25-2.01 (m, 1H), 1.78-1.48 (m, 1H); MS (APCI+) m/z 412.1 [M+H]⁺.

### Example 26: 5-[(7R)-1-fluoro-3-hydroxy-7-{[(3-methyloxetan-3-yl)methyl]amino}-5,6,7,8-tetrahydronaphthalen-2-yl]-1λ⁶,2,5-thiadiazolidine-1,1,3-trione (Compound 344)

The product of Example 26 was prepared using the general procedure described in Example 24, substituting 3-methyloxetane-3-carbaldehyde for 3-thiophenecarboxaldehyde (0.9 mg, 2.5% yield). ¹H NMR (400 MHZ, DMSO-d₆) δ ppm 6.45 (s, 1H), 4.40 (s, 2H), 4.23 (d, J=5.8 Hz, 2H), 3.93 (s, 2H), 3.32 (s, 2H), 3.18-2.58 (m, 5H), 2.21-1.48 (m, 2H), 1.32 (s, 3H); MS (APCI⁺) m/z 400.2 [M+H]⁺.

### Example 27: 5-[(7R)-1-fluoro-3-hydroxy-7-{[(1-methyl-1H-pyrrol-2-yl)methyl]amino}-5,6,7,8-tetrahydronaphthalen-2-yl]-1λ⁶,2,5-thiadiazolidine-1,1,3-trione (Compound 345)

The product of Example 27 was prepared using the general procedure described in Example 24, substituting N-methyl-2-pyrrolecarboxaldehydefor 3-thiophenecarboxaldehyde (0.8 mg, 2.2% yield). ¹H NMR (400 MHz, DMSO-d₆) δ ppm 9.07 (s, 1H), 6.73 (s, 1H), 6.44 (s, 1H), 6.22-5.92 (m, 2H), 3.93 (s, 3H), 3.62 (s, 3H), 3.20-2.96 (m, 3H), 2.77 (s, 3H), 2.24-2.00 (m, 1H), 1.75-1.40 (m, 1H); MS (APCI+) m/z 409.2 [M+H]⁺.

### Example 28: 5-[(7R)-1-fluoro-3-hydroxy-7-{[(1-methyl-1H-pyrrol-3-yl)methyl]amino}-5,6,7,8-tetrahydronaphthalen-2-yl]-1λ⁶,2,5-thiadiazolidine-1,1,3-trione (Compound 346)

The product of Example 28 was prepared using the general procedure described in Example 24, substituting 1-methyl-1H-pyrrole-3-carbaldehyde for 3-thiophenecarboxaldehyde (2.1 mg, 5.7% yield). ¹H NMR (400 MHz, DMSO-d₆) δ ppm 6.86 (d, J=2.0 Hz, 1H), 6.73 (t, J=2.5 Hz, 1H), 6.45 (s, 1H), 6.13 (t, J=2.3 Hz, 1H), 4.01 (s, 2H), 3.93 (s, 2H), 3.61 (s, 3H), 3.17 (d, J=5.1 Hz, 1H), 3.09 (dd, J=15.7, 5.4 Hz, 1H), 2.85-2.68 (m, 2H), 2.56-2.52 (m, 1H), 2.17 (d, J=12.1 Hz, 1H), 1.74-1.62 (m, 1H); MS (APCI+) m/z 409.2 [M+H]⁺.

### Example 29: 5-[(7R)-1-fluoro-3-hydroxy-7-{[(pyridin-3-yl)methyl]amino}-5,6,7,8-tetrahydronaphthalen-2-yl]-1λ⁶,2,5-thiadiazolidine-1,1,3-trione (Compound 347)

The product of Example 29 was prepared using the general procedure described in Example 24, substituting 3-pyridinecarboxaldehyde for 3-thiophenecarboxaldehyde (2.8 mg, 7.6% yield). ¹H NMR (400 MHz, DMSO-d₆) δ ppm 8.56 (d, J=2.2 Hz, 1H), 8.43 (dd, J=4.7, 1.6 Hz, 1H), 7.78 (dt, J=7.8, 2.0 Hz, 1H), 7.33 (dd, J=7.8, 4.7 Hz, 1H), 6.41 (s, 1H), 3.92 (s, 2H), 3.83 (s, 2H), 3.17 (d, J=4.4 Hz, 1H), 2.92-2.70 (m, 4H), 2.28 (dd, J=15.5, 7.6 Hz, 1H). 1.50) (s, 1H); MS (APCI+) m/z 407.1 [M+H]⁺.

### Example 30: 5-{(7R)-1-fluoro-3-hydroxy-7-[(3,3,3-trifluoro-2-methylpropyl)amino]-5,6,7,8-tetrahydronaphthalen-2-yl}-1λ⁶,2,5-thiadiazolidine-1,1,3-trione (Compound 348)

The product of Example 30 was prepared using the general procedure described in Example 24, substituting 2-(trifluoromethyl)propionaldehyde for 3-thiophenecarboxaldehyde (5.7 mg, 14.8% yield). ¹H NMR (400 MHz, DMSO-d₆) δ ppm 6.41 (s, 1H), 3.92 (s, 2H), 2.95-2.52 (m, 6H), 2.47-2.36 (m, 1H), 2.28-2.14 (m, 1H), 1.89 (s, 1H), 1.43 (d, J=11.1 Hz, 1H), 1.11 (d, J=6.7 Hz, 3H); MS (APCI+) m/z 426.1 [M+H]⁺.

### Example 31: 5-[(7R)-1-fluoro-3-hydroxy-7-{[(pyridazin-3-yl)methyl]amino}-5,6,7,8-tetrahydronaphthalen-2-yl]-1λ⁶,2,5-thiadiazolidine-1,1,3-trione (Compound 349)

The product of Example 31 was prepared using the general procedure described in Example 24, substituting pyridazine-3-carbaldehyde for 3-thiophenecarboxaldehyde (6.5 mg, 17.5% yield). ¹H NMR (400 MHZ, DMSO-d₆) δ ppm 9.09 (dd, J=4.9, 1.8 Hz, 1H), 7.81-7.73 (m, 1H), 7.64 (dd, J=8.5, 4.8 Hz, 1H), 6.41 (s, 1H), 4.09 (s, 2H), 3.92 (s, 1H), 2.90-2.69 (m, 4H), 2.54 (s, 1H), 2.35-2.21 (m, 1H), 1.96-1.83 (m, 1H), 1.56-1.42 (m, 1H); MS (APCI+) m/z 408.1 [M+H]⁺.

### Example 32: 5-[(7R)-1-fluoro-3-hydroxy-7-{[(oxan-2-yl)methyl]amino}-5,6,7,8-tetrahydronaphthalen-2-yl]-1λ⁶,2,5-thiadiazolidine-1,1,3-trione (Compound 350)

The product of Example 32 was prepared using the general procedure described in Example 24, substituting tetrahydro-2H-pyran-2-carbaldehyde for 3-thiophenecarboxaldehyde (6.7 mg, 17.8% yield). ¹H NMR (400 MHZ, DMSO-d₆) δ ppm 9.19 (s, 1H), 6.45 (s, 1H), 4.00-3.91 (m, 3H), 3.63-3.53 (m, 1H), 3.46-3.37 (m, 2H), 3.18-2.94 (m, 3H), 2.85-2.65 (m, 2H), 2.58-2.51 (m, 1H), 2.23-2.10 (m, 1H), 1.86-1.76 (m, 1H), 1.70-1.58 (m, 2H), 1.53-1.42 (m, 3H), 1.27-1.19 (m, 1H); MS (APCI+) m/z 414.2 [M+H]⁺.

### Example 33: 5-[(7R)-1-fluoro-3-hydroxy-7-{[(5-methyl-1,2-oxazol-3-yl)methyl]amino}-5,6,7,8-tetrahydronaphthalen-2-yl]-1λ⁶,2,5-thiadiazolidine-1,1,3-trione (Compound 351)

The product of Example 33 was prepared using the general procedure described in Example 24, substituting 5-methyl-1,2-oxazole-3-carbaldehyde for 3-thiophenecarboxaldehyde (9.1 mg, 24.4% yield). ¹H NMR (400 MHZ, DMSO-d₆) δ ppm 6.40 (s, 1H), 6.22 (d, J=1.1 Hz, 1H), 3.92 (s, 2H), 3.76 (s, 2H), 2.87-2.67 (m, 3H), 2.54 (s, 1H), 2.37 (dd, J=5.6, 0.9 Hz, 3H), 2.23 (dd, J=15.8, 8.1 Hz, 1H), 1.83 (s, 1H), 1.53-1.39 (m, 1H); MS (APCI+) m/z 411.1 [M+H]⁺.

### Example 34: 5-[(7R)-1-fluoro-3-hydroxy-7-{[(oxan-3-yl)methyl]amino}-5,6,7,8-tetrahydronaphthalen-2-yl]-1λ⁶,2,5-thiadiazolidine-1,1,3-trione (Compound 352)

The product of Example 34 was prepared using the general procedure described in Example 24, substituting tetrahydro-2H-pyran-3-carbaldehyde for 3-thiophenecarboxaldehyde (10.7 mg, 28.5% yield). ¹H NMR (400 MHZ, DMSO-d₆) δ ppm 6.41 (s, 1H), 3.92 (s, 2H), 3.89-3.68 (m, 3H), 3.33-3.15 (m, 3H), 3.12-3.01 (m, 2H), 2.90-2.76 (m, 2H), 2.66-2.55 (m, 1H), 2.28-2.15 (m, 1H), 1.85-1.37 (m, 5H), 1.26-1.10 (m, 1H); MS (APCI+) m/z 414.2 [M+H]⁺.

### Example 35: 2-({[(2R)-8-fluoro-6-hydroxy-7-(1,1,4-trioxo-1λ⁶,2,5-thiadiazolidin-2-yl)-1,2,3,4-tetrahydronaphthalen-2-yl]amino}methyl)cyclopropane-1-carbonitrile (Compound 353)

The product of Example 35 was prepared using the general procedure described in Example 24, substituting 2-formylcyclopropane-1-carbonitrile for 3-thiophenecarboxaldehyde (12.5 mg, 34.7% yield). ¹H NMR (400 MHz, DMSO-d₆) δ ppm 6.41 (d, J=1.4 Hz, 1H), 3.93 (s, 2H), 2.87-2.56 (m, 6H), 2.25-2.14 (m, 1H), 1.90 (s, 1H), 1.64-1.48 (m, 2H), 1.45-1.40 (m, 1H). 1.18-1.08 (m, 1H), 0.99-0.90 (m, 1H); MS (APCI+) m/z 395.1 [M+H]⁺.

### Example 36: 5-{(7R)-7-[(3-ethoxypropyl)amino]-1-fluoro-3-hydroxy-5,6,7,8-tetrahydronaphthalen-2-yl}-1λ⁶,2,5-thiadiazolidine-1,1,3-trione (Compound 354)

The product of Example 36 was prepared using the general procedure described in Example 24, substituting 3-ethoxypropanal for 3-thiophenecarboxaldehyde (13.3 mg, 36.3% yield). ¹H NMR (400 MHZ, DMSO-d₆) δ ppm 6.41 (s, 1H), 3.93 (s, 2H), 3.45-3.34 (m, 4H), 2.89-2.59 (m, 6H), 2.28-2.17 (m, 1H), 1.94-1.86 (m, 1H), 1.71-1.60 (m, 2H), 1.53-1.39 (m, 1H), 1.09 (t, J=7.0 Hz, 3H); MS (APCI+) m/z 402.3 [M+H]⁺.

### Example 37: 5-[(7R)-7-({[1-(difluoromethyl)cyclopropyl]methyl}amino)-1-fluoro-3-hydroxy-5,6,7,8-tetrahydronaphthalen-2-yl]-1λ⁶,2,5-thiadiazolidine-1,1,3-trione (Compound 355)

The product of Example 37 was prepared using the general procedure described in Example 24, substituting 1-(difluoromethyl)cyclopropane-1-carbaldehyde for 3-thiophenecarboxaldehyde (13.4 mg, 35.2% yield). ¹H NMR (400 MHz, DMSO-d₆) δ ppm 9.19 (s, 1H), 8.90-8.22 (m, 1H), 6.46 (s, 1H), 5.98 (t, J=56.2 Hz, 1H), 3.93 (d, J=1.5 Hz, 2H), 3.25-2.97 (m, 4H), 2.86-2.63 (m, 3H), 2.17 (s, 1H), 1.67 (s, 1H), 0.91 (s, 4H); MS (APCI+) m/z 420.1 [M+H]⁺.

### Example 38: 5-[(7R)-1-fluoro-3-hydroxy-7-{[2-(oxolan-3-yl)ethyl]amino}-5,6,7,8-tetrahydronaphthalen-2-yl]-1λ⁶,2,5-thiadiazolidine-1,1,3-trione (Compound 356)

The product of Example 38 was prepared using the general procedure described in Example 24, substituting 2-(tetrahydrofuran-3-yl)acetaldehyde for 3-thiophenecarboxaldehyde (16.7 mg, 44.4% yield). ¹H NMR (400 MHz, DMSO-d₆) δ ppm 6.42 (s, 1H), 3.93 (s, 2H), 3.82-3.74 (m, 1H), 3.74-3.66 (m, 1H), 3.60 (q, J=7.7 Hz, 1H), 3.21 (t, J=7.7 Hz, 1H), 2.92-2.80 (m, 2H), 2.79-2.56 (m, 4H), 2.21 (tt, J=14.7, 8.2 Hz, 2H), 2.04-1.87 (m, 2H), 1.55-1.37 (m, 4H); MS (APCI+) m/z 414.2 [M+H]⁺.

### Example 39: 5-[(7R)-1-fluoro-3-hydroxy-7-{[(1-methyl-1H-imidazol-5-yl)methyl]amino}-5,6,7,8-tetrahydronaphthalen-2-yl]-1λ⁶,2,5-thiadiazolidine-1,1,3-trione (Compound 357)

The product of Example 39 was prepared using the general procedure described in Example 24, substituting 1-methyl-1H-imidazole-5-carbaldehyde for 3-thiophenecarboxaldehyde (15.4 mg, 41.4% yield). ¹H NMR (400 MHz, DMSO-d₆) δ ppm 7.49 (s, 1H), 6.75 (s, 1H), 6.42 (s, 1H), 3.93 (s, 2H), 3.75 (d, J=1.8 Hz, 2H), 3.60 (s, 3H), 2.90-2.68 (m, 3H), 2.54 (s, 1H), 2.26 (dd, J=15.6, 7.8 Hz, 1H), 1.93 (d, J=11.8 Hz, 1H), 1.55-1.42 (m, 1H); MS (APCI+) m/z 410.1 [M+H]⁺.

### Example 40: 5-[(7R)-7-{[2,2-dimethyl-3-(pyrrolidin-1-yl)propyl]amino}-1-fluoro-3-hydroxy-5,6,7,8-tetrahydronaphthalen-2-yl]-1λ⁶,2,5-thiadiazolidine-1,1,3-trione (Compound 358)

The product of Example 40 was prepared using the general procedure described in Example 24, substituting 2,2-dimethyl-3-pyrrolidin-1-ylpropanal for 3-thiophenecarboxaldehyde (15.1 mg, 37.0% yield). ¹H NMR (400 MHz, DMSO-d₆) δ ppm 6.41 (s, 1H), 3.93 (s, 1H), 3.17 (s, 2H), 2.89-2.69 (m, 2H), 2.62-2.53 (m, 4H), 2.48 (d, J=1.7 Hz, 1H), 2.35 (d, J=1.8 Hz, 1H), 2.31-2.13 (m, 1H), 1.68-1.60 (m, 4H), 1.53-1.38 (m, 1H), 1.07-0.84 (m, 6H); MS (APCI⁺) m/z 455.4 [M+H]⁺.

### Example 41: 5-[(7R)-1-fluoro-3-hydroxy-7-({[5-(hydroxymethyl)furan-2-yl]methyl}amino)-5,6,7,8-tetrahydronaphthalen-2-yl]-1λ⁶,2,5-thiadiazolidine-1,1,3-trione (Compound 359)

The product of Example 41 was prepared using the general procedure described in Example 24, substituting 5-hydroxymethyl-2-furaldehyde for 3-thiophenecarboxaldehyde (13.3 mg, 34.5% yield). ¹H NMR (400 MHz, DMSO-d₆) δ ppm 6.41 (d, J=1.5 Hz, 1H), 6.20-6.13 (m, 2H), 4.33 (s, 2H), 3.93 (s, 2H), 3.74 (s, 2H), 2.88-2.67 (m, 3H), 2.54 (s, 1H), 2.30-2.16 (m, 1H), 1.95-1.88 (m, 1H), 1.55-1.36 (m, 1H); MS (APCI+) m/z 426.1 [M+H]⁺.

### Example 42: 5-{(7R)-1-fluoro-3-hydroxy-7-[(4-methoxybutyl)amino]-5,6,7,8-tetrahydronaphthalen-2-yl}-1λ⁶,2,5-thiadiazolidine-1,1,3-trione (Compound 360)

The product of Example 42 was prepared using the general procedure described in Example 24, substituting 4-methoxy butanal for 3-thiophenecarboxaldehyde (11.2 mg, 30.6% yield). ¹H NMR (400 MHZ, DMSO-d₆) δ ppm 6.41 (d, J=1.5 Hz, 1H), 3.93 (s, 2H), 3.31 (t, J=6.3 Hz, 2H), 3.21 (s, 3H), 2.85-2.80 (m, 1H), 2.79-2.67 (m, 1H), 2.66-2.58 (m, 2H), 2.29-2.18 (m, 1H), 1.95-1.87 (m, 1H), 1.60-1.43 (m, 4H); MS (APCI+) m/z 402.2 [M+H]⁺.

### Example 43: 5-[(7R)-1-fluoro-3-hydroxy-7-{[(oxolan-3-yl)methyl]amino}-5,6,7,8-tetrahydronaphthalen-2-yl]-1λ⁶,2,5-thiadiazolidine-1,1,3-trione (Compound 361)

The product of Example 43 was prepared using the general procedure described in Example 24, substituting tetrahydrofuran-3-carboxaldehyde for 3-thiophenecarboxaldehyde (9.8 mg, 26.9% yield). ¹H NMR (400 MHZ, DMSO-d₆) δ ppm 6.41 (s, 1H), 3.92 (s, 2H), 3.77-3.54 (m, 3H), 3.39 (ddd, J=8.4, 5.9, 2.3 Hz, 1H), 2.87-2.74 (m, 4H), 2.65-2.52 (m, 1H), 2.24 (ddt. J=30.1, 14.6, 7.3 Hz, 3H), 1.99-1.87 (m, 1H), 1.50 (ddd, J=27.1, 13.9, 5.7 Hz, 3H); MS (APCI⁺) m/z 400.2 [M+H]⁺.

### Example 44: 5-[(7R)-7-{[(2,2-difluorocyclopropyl)methyl]amino}-1-fluoro-3-hydroxy-5,6,7,8-tetrahydronaphthalen-2-yl]-1λ⁶,2,5-thiadiazolidine-1,1,3-trione (Compound 362)

The product of Example 44 was prepared using the general procedure described in Example 24, substituting 2,2-difluorocyclopropane-1-carbaldehyde for 3-thiophenecarboxaldehyde (9.1 mg, 24.4% yield). ¹H NMR (400 MHz, DMSO-d₆) δ ppm 6.41 (s, 1H), 3.93 (s, 2H), 2.87-2.79 (m, 2H), 2.77-2.67 (m, 4H), 2.54 (s, 1H), 2.21 (dd, J=17.7, 9.7 Hz, 1H), 1.84 (s, 1H), 1.56-1.41 (m, 2H), 1.19 (dt, J=12.1, 3.9 Hz, 1H); MS (APCI+) m/z 406.1 [M+H]⁺.

### Example 45: 5-{(7R)-1-fluoro-3-hydroxy-7-[(3-methoxypropyl)amino]-5,6,7,8-tetrahydronaphthalen-2-yl}-1λ⁶,2,5-thiadiazolidine-1,1,3-trione (Compound 363)

The product of Example 45 was prepared using the general procedure described in Example 24, substituting 3-methoxypropanal for 3-thiophenecarboxaldehyde (7.8 mg, 22% yield). ¹H NMR (400 MHz, DMSO-d₆) δ ppm 6.41 (s, 1H), 3.92 (s, 2H), 3.37 (t, J=6.4 Hz, 2H), 3.26-3.17 (m, 4H), 2.90-2.59 (m, 3H), 2.21 (d, J=7.5 Hz, 1H), 1.85 (s, 1H), 1.72-1.60 (m, 2H), 1.53-1.31 (m, 1H); MS (APCI+) m/z 388.1 [M+H]⁺.

### Example 46: 5-[(7R)-1-fluoro-3-hydroxy-7-{[(1,3-oxazol-5-yl)methyl]amino}-5,6,7,8-tetrahydronaphthalen-2-yl]-1λ⁶,2,5-thiadiazolidine-1,1,3-trione (Compound 364)

The product of Example 46 was prepared using the general procedure described in Example 24, substituting oxazole-5-carbaldehyde for 3-thiophenecarboxaldehyde (6.0 mg. 16.6% yield). ¹H NMR (400 MHz, DMSO-d₆) δ ppm 8.24 (s, 1H), 7.01 (s, 1H), 6.40 (s, 1H), 3.92 (s, 2H), 3.84 (s, 2H), 2.87-2.69 (m, 4H), 2.61 (dd, J=10.4, 5.7 Hz, 1H), 2.31-2.17 (m, 1H), 1.86 (s, 1H), 1.55-1.37 (m, 1H); MS (APCI+) m/z 397.1 [M+H]⁺.

### Example 47: 5-[(7R)-1-fluoro-3-hydroxy-7-{[2-(oxan-4-yl)ethyl]amino}-5,6,7,8-tetrahydronaphthalen-2-yl]-1λ⁶,2,5-thiadiazolidine-1,1,3-trione (Compound 365)

The product of Example 47 was prepared using the general procedure described in Example 24, substituting 2-(tetrahydro-2H-pyran-4-yl)acetaldehyde for 3-thiophenecarboxaldehyde (5.7 mg, 14.7% yield). ¹H NMR (400 MHz, DMSO-d₆) δ ppm 6.41 (s, 1H), 3.92 (s, 2H), 3.85-3.76 (m, 2H), 3.43 (t, J=6.6 Hz, 1H), 3.26 (tt, J=11.6, 2.4 Hz, 2H), 2.88-2.80 (m, 1H), 2.68-2.60 (m, 1H), 1.85 (s, 2H), 1.65-1.51 (m, 3H), 1.35 (q, J=6.9 Hz, 2H), 1.12 (dd, J=13.7, 9.5 Hz, 2H); MS (APCI+) m/z 428.2 [M+H]⁺.

### Example 48: 5-[(7R)-1-fluoro-3-hydroxy-7-{[(oxetan-3-yl)methyl]amino}-5,6,7,8-tetrahydronaphthalen-2-yl]-1λ⁶,2,5-thiadiazolidine-1,1,3-trione (Compound 366)

The product of Example 48 was prepared using the general procedure described in Example 24, substituting oxetane-3-carbaldehyde for 3-thiophenecarboxaldehyde (5.2 mg, 14.7% yield). ¹H NMR (400 MHz, DMSO-d₆) δ ppm 6.41 (s, 1H), 4.61 (dd, J=7.6, 5.8 Hz, 2H), 4.26 (t, J=5.9 Hz, 2H), 3.92 (s, 2H), 3.06-2.94 (m, 1H), 2.90-2.83 (m, 2H), 2.81-2.55 (m, 4H), 2.20 (dd, J=15.9, 8.1 Hz, 1H), 1.86 (s, 1H), 1.44 (d, J=9.6 Hz, 1H); MS (APCI+) m/z 386.1 [M+H]⁺.

### Example 49: 5-[(7R)-1-fluoro-3-hydroxy-7-{[(1,3-thiazol-2-yl)methyl]amino}-5,6,7,8-tetrahydronaphthalen-2-yl]-1λ⁶,2,5-thiadiazolidine-1,1,3-trione (Compound 367)

The product of Example 49 was prepared using the general procedure described in Example 24, substituting 1,3-thiazole-2-carbaldehyde for 3-thiophenecarboxaldehyde (3.5 mg, 9.3% yield). ¹H NMR (400 MHZ, DMSO-d₆) δ ppm 7.69 (d, J=3.3 Hz, 1H), 7.56 (d, J=3.4 Hz, 1H), 6.41 (s, 1H), 4.09 (s, 2H), 3.92 (s, 2H), 2.92-2.70 (m, 3H), 2.70-2.58 (m, 1H), 2.35-2.22 (m, 1H), 2.01-1.90 (m, 1H), 1.58-1.42 (m, 1H); MS (APCI+) m/z 413.1 [M+H]⁺.

### Example 50: 5-[(7R)-1-fluoro-3-hydroxy-7-{[(pyridazin-4-yl)methyl]amino}-5,6,7,8-tetrahydronaphthalen-2-yl]-1λ⁶,2,5-thiadiazolidine-1,1,3-trione (Compound 368)

The product of Example 50 was prepared using the general procedure described in Example 24, substituting pyridazine-4-carbaldehyde for 3-thiophenecarboxaldehyde (2.4 mg. 6.5% yield). ¹H NMR (400 MHz, DMSO-d₆) δ ppm 9.22 (s, 1H), 9.18-9.08 (m, 1H), 7.65 (dd. J=5.3, 2.3 Hz, 1H), 6.42 (s, 1H), 3.96-3.89 (m, 3H), 3.86 (s, 2H), 2.86-2.81 (m, 2H), 2.75 (d, J=17.1 Hz, 2H), 2.33-2.19 (m, 1H), 1.52-1.48 (m, 1H); MS (APCI+) m/z 408.1 [M+H]⁺.

### Example 51: 5-{(7R)-1-fluoro-3-hydroxy-7-[(3-hydroxybutyl)amino]-5,6,7,8-tetrahydronaphthalen-2-yl}-1λ⁶,2,5-thiadiazolidine-1,1,3-trione (Compound 369)

The product of Example 51 was prepared using the general procedure described in Example 24, substituting 3-hydroxy butanal for 3-thiophenecarboxaldehyde (0.2 mg, 0.6% yield). ¹H NMR (400 MHz, DMSO-d₆) δ ppm 6.46 (s, 1H), 3.93 (s, 2H), 3.22-2.94 (m, 4H), 2.81-2.69 (m, 2H), 2.19-2.07 (m, 2H), 1.77-1.51 (m, 2H), 1.45-0.67 (m, 5H); MS (APCI+) m/z 388.1 [M+H]⁺.

### Example 52: 5-[(7S)-1-fluoro-3-hydroxy-7-[(3-methylbutyl)amino](6,6,7,8,8-²H₅)-5,6,7,8-tetrahydronaphthalen-2-yl]-1λ⁶,2,5-thiadiazolidine-1,1,3-trione (Compound 370)

The enantiomers of the racemic product of Example 22C were obtained by chiral SFC separation. Preparative SFC was performed on the Waters SFC80Q SFC running under ChromScope™ software control. The preparative SFC system was equipped with a CO₂ pump, modifier pump with 4-port solvent selection valve, automated back pressure regulator (ABPR). UV detector, and 6-position fraction collector. The mobile phase was comprised of supercritical CO₂ supplied by a dewar of bone-dry non-certified CO₂ pressurized to 350 psi with a modifier of methanol with diethylamine additive 0.1% v/v at a total flow rate of 80 g/minute. The column was held at ambient temperature and the backpressure regulator was set to maintain 120 bar. The sample was loaded into the modifier stream in 1.5 mL (17 mg) injections. The mobile phase was held isocratically at 55% methanol (0.1% diethylamine):CO₂. Fraction collection was time triggered. The instrument was fitted with a ChiralPak® IC column with dimensions 30×250) mm ID mm length with 5 μm particles. The retention times of the two enantiomers were at 3.7 minutes and 5.25 minutes. The first eluting material was purified by preparative HPLC on a Phenomenex® Luna® 10 μm C18 column (30 mm×250 mm) eluted with a gradient of acetonitrile (A) and water (B) with 0.1% trifluoroacetic acid at a flow rate of 50 mL/minute (0-1 minute 5% A, 1-20 minutes linear gradient 5-80%) to give the title compound (6.3 mg, 0.016 mmol, 34% yield). The enantiomeric excess was determined to be 94.2% using the method described in Example 53. ¹H NMR (500 MHZ, DMSO-d₆) δ ppm 9.27 (brs, 1H), 6.48 (s, 1H), 3.96 (s, 2H), 3.05-2.99 (m, 2H), 2.84-2.69 (m, 2H), 1.67 (dq, J=13.2, 6.6 Hz, 1H), 1.51 (q, J=7.3 Hz, 2H), 0.93 (d, J=6.5 Hz, 6H); MS (ESI⁻) m/z 389 [M−H]⁻.

### Example 53: 5-[(7R)-1-fluoro-3-hydroxy-7-[(3-methylbutyl)amino](6,6,7,8,8-²H₅)-5,6,7,8-tetrahydronaphthalen-2-yl]-1λ⁶,2,5-thiadiazolidine-1,1,3-trione (Compound 371)

The second eluting material from the chiral SFC separated described in Example 52 was purified by preparative HPLC on a Phenomenex® Luna® 10 μm C18 column (30 mm×250 mm) eluted with a gradient of acetonitrile (A) and water (B) with 0.1% trifluoroacetic acid at a flow rate of 50 mL/minute (0)-1 minute 5% A, 1-20 minutes linear gradient 5-80%) to give the title compound (6.5 mg, 35% yield). The enantiomeric excess was determined to be 85.6% by the following method: Analytical SFC was performed on an Agilent 1260 Fusion™ SFC system running under Agilent OpenLab software control. The SFC system included a 6-way column switcher. CO₂ pump, modifier pump, oven, and backpressure regulator. The mobile phase comprised of bulk-delivered bone-dry CO₂ with a modifier mixture of methanol containing 0.1% v/v diethylamine (DEA) additive and CO₂ at a flow rate of 3 mL/minute. The oven temperature was at 35° C. and the outlet pressure at 150 bar. The mobile phase gradient started at 5% modifier and held for 0.1 minutes at a flow rate of 1 mL/minute, then the flow rate was ramped up to 3 mL/minute and held for 0.4 minute. The modifier was ramped from 5% to 50% over the next 8 minutes at 3 mL/minute then held for 1 minute at 50% modifier (3 mL/minute). The gradient was ramped down from 50% to 5% modifier over 0.5 minute (3 mL/minute). The instrument was fitted with a Chiralpak® IC column with dimensions of 4.6 mm i.d.×150 mm length with 5 μm particles. ¹H NMR (500 MHz, DMSO-d₆) δ ppm 9.27 (brs, 1H), 6.48 (s, 1H), 3.96 (s, 2H), 3.05-2.99 (m, 2H), 2.84-2.69 (m, 2H), 1.67 (dq, J=13.2, 6.6 Hz, 1H), 1.51 (q, J=7.3 Hz, 2H), 0.93 (d, J=6.5 Hz, 6H); MS (ESI⁻) m/z 389 [M−H]⁻.

### Example 54: 5-{(3S)-5-fluoro-7-hydroxy-3-[(3-methylbutyl)amino]-3,4-dihydro-2H-1-benzopyran-6-yl}-1λ⁶,2,5-thiadiazolidine-1,1,3-trione (Compound 372)

Isovaleraldehyde (0.06 mL, 0.60 mmol, 3.0 equivalents) was added to a suspension of 5-[(3S)-3-amino-5-fluoro-7-hydroxy-3,4-dihydro-2H-1-benzopyran-6-yl]-1λ⁶,2,5-thiadiazolidine-1,1,3-trione, trifluoroacetic acid salt (nominally 0.2 mmol, 1 equivalent, prepared in Example 55) and triethylamine (0.08 mL, 0.60 mmol, 3.0 equivalents) in 40% ethanol-dichloromethane mixture (v/v, 1.0 mL, 0.2 M) at 23° C. The reaction mixture was stirred for 2 hours at 23° C. Sodium borohydride (30.0 mg, 0.80 mmol, 4.0 equivalents) was added to the reaction mixture in portions at 23° C. The reaction mixture was stirred for 20 minutes at 23° C. The product mixture was diluted carefully with aqueous hydrochloric acid solution (3.0 M, 0.3 mL). The diluted product mixture was partially concentrated under a stream of nitrogen. The partially concentrated mixture was diluted with water (0.2 mL) and dimethyl sulfoxide (0.5 mL). The diluted mixture was purified by reverse phase flash column chromatography (30 g RediSep Rf Gold® C18 column, elution with a gradient of 5-100% methanol-0.025 M aqueous ammonium bicarbonate solution [acidified with solid carbon dioxide]) to furnish the title compound as an ammonia salt (44.0 mg, 54% over three steps). ¹H NMR (400 MHz, DMSO-d₆) δ ppm 9.39 (bs, 1H), 6.20 (s, 1H), 4.29-4.15 (m, 2H), 3.90 (s, 2H), 3.70 (bs, 1H), 3.11-2.96 (m, 3H), 2.76 (d, J=15.5 Hz, 1H), 1.63 (dq, J=13.1, 6.5 Hz, 1H), 1.48 (q, J=7.5 Hz, 2H), 0.95-0.85 (m, 1H), 0.90 (d, J=6.6 Hz, 6H); MS (APCI+) m/z 388 [M+H]⁺.

### Example 55: 5-{(3S)-3-[(4,4-difluorobutyl)amino]-5-fluoro-7-hydroxy-3,4-dihydro-2H-1-benzopyran-6-yl}-1λ⁶,2,5-thiadiazolidine-1,1,3-trione (Compound 373)

Trifluoroacetic acid (1.50 mL, 19.40 mmol, 10.0 equivalents) was added to a suspension of the product of Example 8H (nominally 1.94 mmol, 1 equivalent) in dichloromethane (2.0 mL, ˜1 M) at 23° C. The reaction mixture was stirred for 20 minutes at 23° C. The product mixture was diluted with heptanes (5 mL) and ethyl acetate (2 mL). The diluted mixture was concentrated under a stream of nitrogen. The residue obtained was dried for 1 hour under vacuum at 23° C. to give 5-[(3S)-3-amino-5-fluoro-7-hydroxy-3,4-dihydro-2H-1-benzopyran-6-yl]-1λ⁶,2,5-thiadiazolidine-1,1,3-trione, trifluoroacetic acid salt. MS (APCI⁺) m/z 318 [M+H]⁺.

A solution of 4,4-difluorobutanal in dichloromethane (65% w/v, 0.10 mL, 0.60 mmol. 3.0 equivalents) was added to a suspension of 5-[(3S)-3-amino-5-fluoro-7-hydroxy-3,4-dihydro-2H-1-benzopyran-6-yl]-1λ⁶,2,5-thiadiazolidine-1,1,3-trione, trifluoroacetic acid salt (nominally 0.2 mmol, 1 equivalent) and triethylamine (0.08 mL, 0.60 mmol, 3.0 equivalents) in 40% ethanol-dichloromethane mixture (v/v, 1.0 mL, 0.2 M) at 23° C. The reaction mixture was stirred for 1.5 hours at 23° C. Sodium borohydride (30.0 mg, 0.80 mmol, 4.0 equivalents) was added to the reaction mixture in portions at 23° C. The reaction mixture was stirred for 20 minutes at 23° C. The product mixture was diluted carefully with aqueous hydrochloric acid solution (3.0 M, 0.3 mL). The diluted product mixture was partially concentrated under a stream of nitrogen. The partially concentrated mixture was diluted with water (0.2 mL) and dimethyl sulfoxide (0.5 mL). The diluted mixture was purified by reverse phase flash column chromatography (30 g RediSep Rf Gold C18 column, elution with a gradient of 5-100% methanol-0.025 M aqueous ammonium bicarbonate solution [acidified with solid carbon dioxide]) to furnish the title compound as an ammonia salt (16.5 mg, 20% over three steps). ¹H NMR (400 MHz, DMSO-d₆) δ ppm 6.18 (s, 1H), 4.20-4.03 (m, 2H), 3.89 (s, 2H), 3.07-2.91 (m, 2H), 2.71-2.58 (m, 1H), 1.97-1.80 (m, 2H), 1.75-1.58 (m, 2H), 1.29-1.01 (m, 2H); MS (APCI+) m/z 410 [M+H]⁺.

### Example 56: 5-{(7R)-7-[(5-amino-3,3-dimethylpentyl)amino]-1-fluoro-3-hydroxy-5,6,7,8-tetrahydronaphthalen-2-yl}-1λ⁶,2,5-thiadiazolidine-1,1,3-trione (Compound 374)

### Example 56A: tert-butyl (5-hydroxy-3,3-dimethylpentyl)carbamate

To a solution of 5-amino-3,3-dimethyl-5-oxopentanoic acid (prepared according to reported method in J. Am. Chem. Soc. 1984, 106, 4814-4818) (16.5 g, 93 mmol, 90% pure) in tetrahydrofuran (450) mL) was added lithium aluminum hydride (14.16 g, 373 mmol) in portions at 0° C., and the mixture was heated at 70° C. for 12 hours. The reaction mixture was then cooled down to 0° C.) and quenched by carefully adding water (14 mL), a 15 weight % aqueous solution of NaOH (14 mL) and water (42 mL) successively.

To this mixture was added triethylamine (52.0 mL, 373 mmol) followed by di-tert-butyl dicarbonate (43.3 mL, 187 mmol) at 0° C. The resulting mixture was stirred for 3 hours at 20° C. The reaction mixture was filtered and the solid residue was washed with ethyl acetate (3×100 mL). The filtrate was concentrated under reduced pressure, and the residue was purified by column chromatography on silica gel eluting with 2-5% ethyl acetate in petroleum ether to give the title compound (8.2 g, 90% pure, 34% yield). ¹H NMR (400 MHZ, CDCl₃) δ ppm 4.51 (br s, 1H), 3.72 (br t, J=7.32 Hz, 2H), 3.14 (m, 2H), 1.36-1.60 (m, 13H), 0.94 (s, 6H).

### Example 56B: tert-butyl (5-{[tert-butyl(dimethyl)silyl]oxy}-3,3-dimethylpentyl)carbamate

To a solution of Example 56A (4 g, 15.56 mmol, 90% pure) in dichloromethane (80) mL) was added imidazole (2.119 g, 31.1 mmol) followed by tert-butyldimethylchlorosilane (3.52 g, 23.34 mmol) at 0° C. and the mixture was stirred at 0° C. for 2 hours. The mixture was then quenched with water (50 mL) and extracted with dichloromethane (3×50 mL). The combined organic fractions were washed with brine (10 mL), dried over Na₂SO₄, and filtered. The filtrate was concentrated to give the title compound (6 g, 90% pure, 100% yield). ¹H NMR (400 MHZ, CDCl₃) δ ppm 3.60-3.7 (t, J=7.2 Hz, 2H), 3.13 (br s, 2H), 1.47-1.41 (m, 14H), 0.91-0.88 (m, 15H), 0.06 (s, 6H).

### Example 56C: di-tert-butyl (5-{[tert-butyl(dimethyl)silyl]oxy}-3,3-dimethylpentyl)-2-imidodicarbonate

The title compound (10 g, mixed with di-tert-butyl decarbonate, estimated 40% pure by ¹H NMR) was prepared from Example 56B (6 g) by the same method as described for Example 58C. ¹H NMR (400 MHZ, CDCl₃) δ ppm 3.73-3.66 (m, 2H), 3.61-3.53 (m, 2H), 1.50-1.42 (m, 99H), 0.89 (s, 9H), 0.07-0.02 (m, 6H).

### Example 56D: di-tert-butyl (5-hydroxy-3,3-dimethylpentyl)-2-imidodicarbonate

To a solution of Example 56C (4 g, 3.59 mmol, ˜40% pure) in tetrahydrofuran (40 mL) was added tetrabutylammonium fluoride (1 mol/L in tetrahydrofuran, 3.59 mL, 3.59 mmol) at 20° C. and the mixture was stirred at 20° C. for 12 hours. The reaction mixture was diluted with water (200 mL) and extracted with ethyl acetate (200 mL). The organic fraction was washed with brine (2×50 mL), dried over anhydrous Na₂SO₄, filtered, and the filtrate was concentrated under reduced pressure. The residue was purified by column chromatography on silica gel eluted with 20-25% ethyl acetate in petroleum ether to give the title compound (1.2 g, 91% yield).

### Example 56E: di-tert-butyl (3,3-dimethyl-5-oxopentyl)-2-imidodicarbonate

To a solution of Example 56D (1.3 g, 3.53 mmol) in dichloromethane (20 mL) at 0° C. was added 1,1,1-tris (acetyloxy)-1,1-dihydro-1,2-benzodioxol-3-(1H)-one (2.25 g, 5.29 mmol) in portions at 0° C. The resulting mixture was stirred at 0° C. for 2 hours. The reaction mixture was then quenched with water (30 mL) and filtered through a pad of diatomaceous earth. The filter cake was washed with dichloromethane (2×10 mL). The filtrate and wash were transferred to a separatory funnel, and the organic phase was separated, washed with brine (5 mL), dried over anhydrous Na₂SO₄, filtered, and the filtrate was concentrated in vacuo. The residue was purified by silica gel column chromatography eluted with 2%-5% of ethyl acetate in petroleum ether to afford the title compound (1.1 g, yield 85%). ¹HNMR (400 MHZ, CDCl₃) δ ppm 9.87 (t, J=3.00 Hz, 1H), 3.59-3.66 (m, 2H), 2.31 (d, J=3.00 Hz, 2H), 1.61-1.68 (m, 2H), 1.51 (s, 18H), 1.11 (s, 6H).

### Example 56F: di-tert-butyl (5-{[(2R)-6-(benzyloxy)-8-fluoro-7-(1,1,4-trioxo-1λ⁶,2,5-thiadiazolidin-2-yl)-1,2,3,4-tetrahydronaphthalen-2-yl]amino}-3,3-dimethylpentyl)-2-imidodicarbonate

The title compound was prepared from the product of Example 56E and the product of Example 20G by the same method as described for Example 58E. MS (ESI⁻) m/z 717.2 [M−H]⁻.

### Example 56G: 5-{(7R)-7-[(5-amino-3,3-dimethylpentyl)amino]-1-fluoro-3-hydroxy-5,6,7,8-tetrahydronaphthalen-2-yl}-1λ⁶,2,5-thiadiazolidine-1,1,3-trione

A mixture of Example 56F (350 mg, 0.341 mmol, 70% pure), 10% Pd—C (181 mg, 0.170 mmol) and 1 M aqueous HCl (0.5 mL, 0.5 mmol) in methanol (35 mL) was stirred under H₂ (15 psi) at 20° C. for 12 hours. The reaction mixture was filtered and the cake was washed with methanol (2×25 mL). The filtrate was neutralized to pH=6-7 with solid NaHCO₃, filtered and concentrated under reduced pressure. The residue was diluted with ethyl acetate (20 mL) and treated with a 4 M solution of HCl in ethyl acetate solution (20 mL) at 20° C. for 2 hours. The reaction mixture was neutralized to pH=6-7 with solid NaHCO₃, filtered, and the filtrate was concentrated under reduced pressure. The residue was purified by preparative HPLC on Welch Xtimate® C18 column (100×25 mm, 3 μm) eluted with acetonitrile (A) in water with 0.04% HCl (B) (gradient: 1%-10% A for 8 minutes, 10-100% of A for 0.1 minute, and 100% of A for 2 minutes) at a flow rate of 25 mL/minute to give the title compound (48 mg, 27.1% yield) as bis HCl salt. ¹H NMR (400 MHZ, methanol-d₄) δ ppm 6.57 (s, 1H), 4.42 (s, 2H), 3.55 (m, 1H), 3.31 (m, 1H), 3.15-3.23 (m, 2H), 2.96-3.04 (m, 2H), 2.88-2.95 (m, 2H), 2.67 (dd, J=16.07, 10.19 Hz, 1H), 2.34 (m, 1H), 1.84 (m, 1H), 1.60-1.75 (m, 4H), 1.06 (s, 6H); MS (ESI⁻) m/z 427[M−H]⁻.

### Example 57: 5-[(3S)-5-fluoro-7-hydroxy-3-{[2-(oxan-4-yl)ethyl]amino}-3,4-dihydro-2H-1-benzopyran-6-yl]-1λ⁶,2,5-thiadiazolidine-1,1,3-trione (Compound 375)

The product of Example 8I (43.1 mg, 0.10 mmol, 1.0 equivalent) was dissolved in 1.0 mL of acetic acid/sodium acetate buffer in methanol (adjusted to pH=4.5). 2-(Tetrahydro-2H-pyran-4-yl)acetaldehyde (0.6 M in methanol, 216 μL, 0.13 mmol, 1.3 equivalents) was added, and the mixture was stirred at room temperature for 1 hour. To the vessel was added MP-CNBH₃ resin (137 mg, 3 equivalents, 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40 mL/minute (0-0.5 minutes 5% A, 0.5-8.0 minutes linear gradient 5-80% A, 8.0-8.1 minutes linear gradient 80-100% A, 8.1-9.0 minutes 100% A, 9.0-9.1 minutes linear gradient 100-5% A. 9.1-10.0 minutes 5% A) to afford the title compound (9.3 mg, 17% yield). ¹H NMR (400 MHz, DMSO-d₆) δ ppm 8.64 (s, 1H), 6.24 (s, 1H), 4.35-4.26 (m, 1H), 4.25-4.17 (m, 1H), 4.08 (s, 2H), 3.88-3.74 (m, 3H), 3.30-3.22 (m, 2H), 3.14-3.01 (m, 3H), 2.80 (dd, J=17.3, 4.4 Hz, 1H), 1.62-1.50 (m, 5H), 1.26-1.14 (m, 2H); MS (APCI+) m/z 430.2 [M+H]⁺.

### Example 58: 5-[(7R)-7-({2-[1-(aminomethyl)cyclobutyl]ethyl}amino)-1-fluoro-3-hydroxy-5,6,7,8-tetrahydronaphthalen-2-yl]-1λ⁶,2,5-thiadiazolidine-1,1,3-trione (Compound 376)

### Example 58A: 1-(prop-2-en-1-yl)cyclobutane-1-carboxamide

To a solution of 1-allylcyclobutanecarboxylic acid (prepared according to Journal of Medicinal Chemistry, 2010, 53(6), 2666-2670) (14 g, 80 mmol, 80% pure) and N,N-dimethylformamide (58 mg, 0.799 mmol) in dichloromethane (200 mL) was added oxalyl chloride (12.17 g, 96 mmol) dropwise at 0° C. The mixture was stirred at 20° C. for 2 hours. The reaction mixture was added dropwise into 30% NH₃≮H₂O (200 mL) at 0° C. After the addition, the reaction mixture was stirred at 20° C. for 2 hours. The reaction mixture was then filtered through a pad of diatomaceous earth, and the cake was washed with dichloromethane (2×500 mL). The filtrate was transferred to a separatory funnel, and the organic phase was separated and washed with brine (100 mL), dried over anhydrous sodium sulfate, filtered and the filtrate was concentrated in vacuo to afford the title compound (13 g, 75% pure, 95% yield), which was used for the next step without further purification.

### Example 58B: tert-butyl {[1-(prop-2-en-1-yl)cyclobutyl]methyl}carbamate

To a solution of Example 58A (10 g, 53.9 mmol, 75% pure) in tetrahydrofuran (300) mL) at 0° C. was added lithium aluminum hydride (2.454 g, 64.7 mmol) in portions at 0° C. The resulting mixture was heated to 70° C. and stirred for 12 hours. The reaction mixture was slowly quenched with water (3 mL) followed by 15 weight % aqueous NaOH (3 mL) and additional water (9 mL). To the resulting mixture, triethylamine (8.18 g, 81 mmol) was added at 0° C. followed by di-tert-butyl dicarbonate (14.12 g, 64.7 mmol). The resulting mixture was stirred at 20° C. for 12 hours. The reaction mixture was then filtered through a pad of diatomaceous earth, and the cake was washed with ethyl acetate (2×50 mL). The filtrate was concentrated under reduced pressure. The residue was diluted with water (100 mL) and ethyl acetate (200 mL), and the resulting biphasic mixture was separated. The organic phase was washed with brine (20) mL), dried over anhydrous sodium sulfate, filtered, and the filtrate was concentrated under reduced pressure. The residue was purified by column chromatography on silica gel eluted with ethyl acetate in petroleum ether from 5% to 10% to afford the title compound (10 g, 90% pure. 74.1% yield for two steps). ¹H NMR (400 MHZ, CDCl₃) δ ppm 5.91-5.66 (m, 1H), 5.15-4.98 (m, 2H), 3.24-3.09 (m, 2H), 2.25-2.14 (m, 2H), 1.97-1.71 (m, 6H), 1.51-1.43 (m, 9H).

### Example 58C: di-tert-butyl {[1-(prop-2-en-1-yl)cyclobutyl]methyl}-2-imidodicarbonate

To a solution of Example 58B (6 g, 23.97 mmol, 90% pure) in di-tert-butyl dicarbonate (106 mL, 458 mmol) at 20° C. was added 4-dimethylaminopyridine (5.86 g, 47.9 mmol) in portions and the resulting mixture was stirred at 20° C. for 12 hours. The reaction mixture was then diluted with water (300 mL) and extracted with ethyl acetate (200 mL). The organic fraction was washed with brine (2×50 mL), dried over anhydrous sodium sulfate, filtered, and the filtrate was concentrated under reduced pressure. The residue was purified by column chromatography on silica gel eluted with ethyl acetate in petroleum ether from 5% to 10% to afford a mixture of the title compound (15 g, 50% pure by ¹HNMR, yield 96%) and di-tert-butyl dicarbonate, which was used in the next step without further purification. ¹H NMR (400 MHz, CDCl₃) δ ppm 5.95-5.78 (m, 1H), 5.11-5.04 (m, 2H), 3.59 (s, 2H), 2.23 (d, J=7.3 Hz, 2H), 1.99-1.90 (m, 2H), 1.83-1.73 (m, 2H), 1.70-1.64 (m, 2H), 1.49-1.44 (m, 18H).

### Example 58D: di-tert-butyl {[1-(2-oxoethyl)cyclobutyl]methyl}-2-imidodicarbonate

To a solution of Example 58C (5 g, 7.68 mmol, 50% pure) in dioxane-water (10:1, 220 mL) was added a solution of osmium tetroxide in t-butanol (0.2 mol/L, 150 mg, 0.590 mmol) dropwise at 20° C. The mixture was stirred at 20° C. for 15 minutes before sodium periodate (6.57 g, 30.7 mmol) was added in portions at 0° C. and the resulting mixture was stirred at 20° C. for 2 hours. The mixture was diluted with ethyl acetate (200 mL), filtered and the filtrate was treated with saturated sodium thiosulfate aqueous solution (300 mL) at 20° C. for 20 minutes. The mixture was then extracted with ethyl acetate (3×100 mL). The combined organic phases were washed with brine (200 mL), dried over anhydrous sodium sulfate, filtered and the filtrate was concentrated under reduced pressure. The residue was purified by column chromatography on silica gel eluted with petroleum ether:ethyl acetate (50:1) to give a mixture of the title compound (2.5 g, 50% pure, 50% yield) and di-tert-butyl dicarbonate, which was used in the next step without further purification. ¹H NMR (400 MHZ, CDCl₃) δ ppm 1.50 (s, 18H), 1.77-1.86 (m, 2H), 1.87-1.96 (m, 2H), 2.04-2.13 (m, 2H), 2.58 (d, J=2.01 Hz, 2H), 3.85 (s, 2H), 9.79 (t, J=2.07 Hz, 1H).

### Example 58E: di-tert-butyl {[1-(2-{[(2R)-6-(benzyloxy)-8-fluoro-7-(1,1,4-trioxo-1λ⁶,2,5-thiadiazolidin-2-yl)-1,2,3,4-tetrahydronaphthalen-2-yl]amino}ethyl)cyclobutyl]methyl}-2-imidodicarbonate

To a solution of Example 20G (300 mg, 0.577 mmol, 85% pure) in ethanol (6 mL) and dichloromethane (9 mL) was added triethylamine (175 mg, 1.731 mmol), and the mixture was stirred at 20° C. for 3 minutes. Then Example 58D (1511 mg, 2.308 mmol, 50% pure) was added dropwise at 20° C. After the addition, the mixture was stirred at 20° C. for 2 hours. NaBH₄ (sodium borohydride) (92 mg, 2.424 mmol) was then added to the mixture at 0° C. in portions and the resulting mixture was stirred at 20° C. for 20 minutes. The mixture was quenched with 5 mL methanol at 0° C. and stirred for 10 minutes. The mixture was diluted with water (20 mL) and filtered. The filtrate was extracted with dichloromethane (2×15 mL). The combined organic phases were dried over anhydrous sodium sulfate, filtered, and the filtrate was concentrated under reduced pressure to give the crude material which was triturated with petroleum ether; ethanol=30:1 (2 mL) to give the title compound (350 mg, 90% pure, 76% yield). ¹H NMR (400 MHZ, DMSO-d₆) δ ppm 8.38-8.66 (m, 1H), 7.49 (d, J=6.97 Hz, 2H), 7.26-7.39 (m, 3H), 6.74 (s, 1H), 6.74 (s, 1H), 5.12 (s, 2H), 3.95 (d, J=2.08 Hz, 2H), 3.02-3.20 (m, 3H), 2.71-2.91 (m, 2H), 2.58 (br dd, J=16.26, 9.90 Hz, 1H), 2.18 (br d, J=10.76 Hz, 1H), 1.58-1.94 (m, 10H), 1.40-1.50 (m, 18H).

### Example 58F: 5-[(7R)-7-({2-[1-(aminomethyl)cyclobutyl]ethyl}amino)-1-fluoro-3-hydroxy-5,6,7,8-tetrahydronaphthalen-2-yl]-1λ⁶,2,5-thiadiazolidine-1,1,3-trione

To a suspension of Example 58E (350 mg, 90% pure, 0.439 mmol.) in methanol (10) mL) and 1 M HCl aqueous solution (1 mL) was added 10% Pd/C (46.8 mg, 0.439 mmol) and the mixture was stirred under H₂ (15 psi) at 20° C. for 2 hours. The mixture was filtered, and the filtrate was neutralized to pH 7 by adding NaHCO₃ solid in portions at 0° C. The resulting mixture was concentrated under reduced pressure to give solids which were suspended in ethyl acetate (3 mL) and treated with 4 M hydrogen chloride in ethyl acetate (3 mL) dropwise at 0° C.) The mixture was then neutralized again to pH 7 by adding NaHCO₃ (solid) in portions at 0° C.) The resulting mixture was then concentrated under reduced pressure. The residue was purified by preparative HPLC (column: Waters Xbridge BEH C18, 100×30 mm, 10 μm; flow rate: 25 mL/minute, 1-30% gradient of acetonitrile in buffer (10 mM NH₄HCO₃ in H₂O) to give the title compound (99 mg, 52% yield). ¹H NMR (400 MHz, DMSO-d₆) δ ppm 6.43 (s, 1H), 3.93 (s, 2H), 2.78-2.99 (m, 5H), 2.55-2.78 (m, 5H), 2.22-2.36 (m, 1H), 1.94-2.04 (m, 1H), 1.65-1.92 (m, 8H), 1.46 (m, 1H); MS (ESI⁻); m/z 425 [M−H]⁻.

### Example 59: 5-[(7R)-7-({2-[1-(2-aminoethyl)cyclobutyl]ethyl}amino)-1-fluoro-3-hydroxy-5,6,7,8-tetrahydronaphthalen-2-yl]-1λ⁶,2,5-thiadiazolidine-1,1,3-trione (Compound 377)

### Example 59A: [1-(prop-2-en-1-yl)cyclobutyl]methyl methanesulfonate

To a solution of (1-allylcyclobutyl)methanol (prepared according to Bioorganic and Medicinal Chemistry, 2002, 10 (4), 1093-1106) (5 g, 31.7 mmol) and triethylamine (6.63 mL, 4.81 g, 47.5 mmol) in dichloromethane (100 mL) was added methanesulfonyl chloride (4.1 g, 35.8 mmol) dropwise at 0° C. The resulting mixture was stirred at 0° C.) for 1 hour. The reaction mixture was quenched with water (50 mL). The resulting biphasic mixture was separated and the organic phase was washed with brine (10 mL), dried over anhydrous Na₂SO₄, filtered, and the filtrate was concentrated to afford the title compound (8 g, crude) which was used for the next step without further purification. ¹H NMR (400 MHZ, CDCl₃) δ ppm 1.83-1.95 (m, 6H), 2.30 (d, J=7.38 Hz, 2H), 3.01 (s, 3H), 4.10-4.15 (m, 2H), 5.07-5.15 (m, 2H), 5.70-5.82 (m, 1H).

### Example 59B: [1-(prop-2-en-1-yl)cyclobutyl]acetonitrile

To a solution of Example 59A (8 g, 39.2 mmol) in N,N-dimethylformamide (100 mL) at 20° C. was added sodium cyanide (3.76 g, 77 mmol) in portions at 20° C. and the resulting mixture was stirred at 60° C. for 14 hours. The reaction mixture was diluted with water (400 mL) and extracted with ethyl acetate (400 mL). The organic fraction was washed with brine (4×50) mL), dried over anhydrous Na₂SO₄, filtered, and the filtrate was concentrated under reduced pressure. The residue was diluted with methyl tert-butyl ether (30 mL), then filtered through a pad of silica (3 cm×5 cm), and the cake was washed with methyl tert-butyl ether (5×100 mL). The filtrate was concentrated under reduced pressure to afford the title compound (8 g, 60% pure, 82% yield). ¹H NMR (400 MHZ, CDCl₃) δ ppm 1.87-1.98 (m, 6H), 2.34 (d, J=7.38 Hz, 2H), 2.41 (s, 2H), 3.21 (s, 1H), 5.10-5.20 (m, 2H), 5.73 (ddt, J=17.15, 9.99, 7.32 Hz, 1H).

### Example 590; tert-butyl {2-[1-(prop-2-en-1-yl)cyclobutyl]ethyl}carbamate

To a solution of Example 59B (7.3 g, 32.4 mmol, purity is 60%) in tetrahydrofuran (100 mL) was added lithium aluminum hydride (1.475 g, 38.9 mmol) in portions at 0° C. The resulting mixture was stirred at 0° C. for 1 hour. The reaction mixture was slowly quenched with water (2 mL), 2 mL of 15% NaOH solution and 6 mL of water sequentially. The resulting mixture was filtered, and the filter cake was washed with tetrahydrofuran (100 mL). To the filtrate was added triethylamine (9.01 mL, 64.6 mmol) followed by di-tert-butyl dicarbonate (12.01 mL, 51.7 mmol) at 20° C. and the resulting mixture was stirred at 20° C. for 12 hours. The reaction mixture was filtered through a pad of diatomaceous earth and the filter cake was washed with ethyl acetate (2×50 mL). The combined filtrate was concentrated under reduced pressure. The residue was diluted with water (300 mL) and extracted with ethyl acetate (200 mL). The organic fraction was washed with brine (2×50 mL), dried over anhydrous Na₂SO₄, filtered, and the filtrate was concentrated under reduced pressure to give the title compound (4 g, 90% pure, 56.4% yield). ¹H NMR (400 MHZ, CDCl₃) δ ppm 1.40-1.51 (m, 9H), 1.56-1.66 (m, 2H), 1.74-1.91 (m, 6H), 2.20 (br d, J=7.13 Hz, 2H), 2.98-3.19 (m, 2H), 4.97-5.14 (m, 2H), 5.66-5.88 (m, 1H).

### Example 59D: di-tert-butyl {2-[1-(prop-2-en-1-yl)cyclobutyl]ethyl}-2-imidodicarbonate

The title compound was prepared in 32% yield from Example 59C using the procedure described for Example 58C. ¹H NMR (400 MHZ, CDCl₃) δ ppm 5.91-5.71 (m, 1H), 5.15-5.00 (m, 2H), 3.61-3.48 (m, 2H), 2.18 (d, J=7.3 Hz, 2H), 1.91-1.76 (m, 6H), 1.74-1.67 (m, 2H), 1.52 (s, 18H).

### Example 59E: 5-(N,N-di-tert-butoxycarbonyl)amino-3,3-cyclobutylpentanal

The title compound was prepared in 77% yield from Example 59D, using the procedure described for Example 58D. ¹H NMR (400 MHZ, CDCl₃) δ ppm 1.48-1.57 (m, 18H), 1.82-1.90 (m, 3H), 1.92-2.04 (m, 6H), 2.54-2.63 (m, 2H), 3.51-3.62 (m, 2H), 9.83 (t, J=2.56 Hz, 1H).

### Example 59F: di-tert-butyl {2-[1-(2-{[(2R)-6-(benzyloxy)-8-fluoro-7-(1,1,4-trioxo-1λ⁶,2,5-thiadiazolidin-2-yl)-1,2,3,4-tetrahydronaphthalen-2-yl]amino}ethyl)cyclobutyl]ethyl}-2-imidodicarbonate

The title compound was prepared in 83% yield from Example 59E using the procedure described for Example 58E. ¹H NMR (400 MHz, DMSO-d₆) δ ppm 1.40-1.51 (m, 21H), 1.60-1.67 (m, 2H), 1.72-1.91 (m, 10H), 2.12-2.22 (m, 1H), 2.54-2.62 (m, 1H), 2.59 (br d, J=15.76 Hz, 2H), 2.72-2.89 (m, 3H), 2.91-3.01 (m, 2H), 3.13 (br d, J=12.76 Hz, 1H), 3.40-3.50 (m, 2H), 3.90-3.99 (m, 2H), 5.12 (s, 2H), 6.74 (s, 1H), 7.26-7.40 (m, 3H), 7.49 (d, J=7.13 Hz, 2H), 8.26-8.74 (m, 1H).

### Example 59G: 5-[(7R)-7-({2-[1-(2-aminoethyl)cyclobutyl]ethyl}amino)-1-fluoro-3-hydroxy-5,6,7,8-tetrahydronaphthalen-2-yl]-1λ⁶,2,5-thiadiazolidine-1,1,3-trione

A mixture of Example 59F (100 mg, 0.123 mmol) and 10% Pd—C (13.10 mg, 0.123 mmol) in methanol (5 mL) and 1 M aqueous HCl (0.5 mL,) was stirred under H₂ (15 psi) at 20° C. for 12 hours. The mixture was filtered, and the filtrate was neutralized to pH=7 by adding NaHCO₃ in portions at 0° C. The resulting mixture was filtered to remove the solid residues and the filtrate was concentrated under reduced pressure. The residue was dissolved in ethyl acetate (2 mL) and treated with a solution of hydrogen chloride in ethyl acetate (2 mL, 4 mol/L) dropwise at 0° C.) After the mixture was stirred at 25° C. for 2 hours, the mixture was cooled to 0° C. and sodium bicarbonate was added to the mixture in portions to adjust pH to 7. The resulting mixture was concentrated under reduced pressure. The resultant residue was purified by preparative HPLC [column: Welch Xtimate® C18 (100×25 mm, 3 μm), flow rate: 25 mL/minute. 1-10% gradient of acetonitrile in buffer (0.04% HCl aqueous solution) for 20 minutes] to give the title compound as the bis-hydrochloric acid salt (12 mg, yield 17%). ¹H NMR (400 MHz, DMSO-d₆) δ ppm 1.64-1.92 (m, 10H), 2.06-2.09 (m, 2H), 2.23 (m, 1H), 2.57-3.00 (m, 7H), 3.13 (m, 1H), 4.14 (s, 2H), 6.51 (s, 1H), 7.92 (br s, 1H), 7.83 (br s, 2H), 8.93-9.18 (m, 2H), 9.73 (br s, 1H); MS (ESI⁻) m/z 439 [M−H]⁻.

### Example 60: 5-[(3S)-5-fluoro-7-hydroxy-3-{[2-(2,6,6-trimethylcyclohex-1-en-1-yl)ethyl]amino}-3,4-dihydro-2H-1-benzopyran-6-yl]-1λ⁶,2,5-thiadiazolidine-1,1,3-trione (Compound 378)

The product of Example 81 (43.1 mg, 0.10 mmol, 1.0 equivalent) was dissolved in 1.0 mL of acetic acid/sodium acetate buffer in methanol (adjusted to pH=4.5). 2-(2,6,6-Trimethylcyclohex-1-en-1-yl)acetaldehyde (0.6 M in methanol, 216 μL, 0.13 mmol, 1.3 equivalents) was added, and the mixture was stirred at room temperature for 1 hour. To the vessel was added MP-CNBH₃ resin (137 mg, 3 equivalents, 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40 mL/minute (0-0.5 minutes 5% A, 0.5-8.0 minutes linear gradient 5-80% A, 8.0-8.1 minutes linear gradient 80-100% A, 8.1-9.0 minutes 100% A, 9.0-9.1 minutes linear gradient 100-5% A, 9.1-10.0 minutes 5% A) to afford the title compound (5.2 mg, 11% yield). ¹H NMR (400 MHz, DMSO-d₆) δ ppm 6.21 (s, 1H), 4.24-4.13 (m, 2H), 3.90 (d, J=1.2 Hz, 2H), 3.07-2.97 (m, 1H), 2.93-2.85 (m, 2H), 2.76-2.67 (m, 1H), 2.36-2.29 (m, 2H), 1.93-1.85 (m, 2H), 1.62 (s, 3H), 1.58-1.47 (m, 2H), 1.43-1.36 (m, 2H), 1.26-1.16 (m, 1H), 0.99 (s, 6H); MS (APCI+) m/z 468.4 [M+H]⁺.

### Example 61: 5-[(3S)-3-{[3-(2,2-difluoroethoxy)propyl]amino}-5-fluoro-7-hydroxy-3,4-dihydro-2H-1-benzopyran-6-yl]-1λ⁶,2,5-thiadiazolidine-1,1,3-trione (Compound 379)

The product of Example 8I (43.1 mg, 0.10 mmol, 1.0 equivalent) was dissolved in 1.0 mL of acetic acid/sodium acetate buffer in methanol (adjusted to pH=4.5). 3-(2,2-Difluoroethoxy)propanal (0.6 M in methanol, 216 μL, 0.13 mmol, 1.3 equivalents) was added, and the mixture was stirred at room temperature for 1 hour. To the vessel was added MP-CNBH₃ resin (137 mg, 3 equivalents. 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40 mL/minute (0-0.5 minutes 5% A, 0.5-8.0 minutes linear gradient 5-80% A, 8.0-8.1 minutes linear gradient 80-100% A, 8.1-9.0 minutes 100% A, 9.0-9.1 minutes linear gradient 100-5% A. 9.1-10.0 minutes 5% A) to afford the title compound (6.0 mg, 11% yield). ¹H NMR (400 MHz, DMSO-d₆) δ ppm 8.69 (s, 1H), 6.31-6.00 (m, 2H), 4.35-4.27 (m, 1H), 4.24-4.16 (m, 1H), 4.08 (s, 2H), 3.82-3.77 (m, 1H), 3.75-3.56 (m, 4H), 3.15-3.01 (m, 3H), 2.84-2.74 (m, 1H). 1.88 (p, J=6.6 Hz, 2H); MS (APCI+) m/z 440.2 [M+H]⁺.

### Example 62: 5-[(3S)-5-fluoro-7-hydroxy-3-({[4-(trifluoromethyl)cyclohexyl]methyl}amino)-3,4-dihydro-2H-1-benzopyran-6-yl]-1λ⁶,2,5-thiadiazolidine-1,1,3-trione (Compound 380)

The product of Example 8I (43.1 mg, 0.10 mmol, 1.0 equivalent) was dissolved in 1.0 mL of acetic acid/sodium acetate buffer in methanol (adjusted to pH=4.5). 4-(Trifluoromethyl)cyclohexane-1-carbaldehyde (0.6 M in methanol, 216 μL, 0.13 mmol, 1.3 equivalents) was added, and the mixture was stirred at room temperature for 1 hour. To the vessel was added MP-CNBH₃ resin (137 mg, 3 equivalents, 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40 mL/minute (0-0.5 minutes 5% A, 0.5-8.0 minutes linear gradient 5-80% A, 8.0-8.1 minutes linear gradient 80-100% A, 8.1-9.0 minutes 100% A, 9.0-9.1 minutes linear gradient 100-5% A, 9.1-10.0 minutes 5% A) to afford the title compound (7.7 mg, 13% yield). ¹H NMR (400 MHz, DMSO-d₆) δ ppm 6.24 (s, 1H), 4.36-4.23 (m, 2H), 4.15 (s, 2H), 3.81-3.69 (m, 1H), 3.19-3.02 (m, 3H), 2.96 (s, 1H), 2.82 (ddd, J=17.1, 12.1, 5.3 Hz, 1H), 2.29 (d, J=24.7 Hz, 1H), 1.91 (d, J=11.8 Hz, 2H), 1.70-1.41 (m, 4H), 1.30-0.91 (m, 2H); MS (APCI+) m/z 481.9 [M+H]⁺.

### Example 63: 5-[(3S)-5-fluoro-3-({[1-(fluoromethyl)cyclopropyl]methyl}amino)-7-hydroxy-3,4-dihydro-2H-1-benzopyran-6-yl]-1λ⁶,2,5-thiadiazolidine-1,1,3-trione (Compound 381)

The product of Example 81 (43.1 mg, 0.10 mmol, 1.0 equivalent) was dissolved in 1.0 mL of acetic acid/sodium acetate buffer in methanol (adjusted to pH=4.5). 1-(Fluoromethyl)cyclopropane-1-carbaldehyde (0.6 M in methanol, 216 μL, 0.13 mmol, 1.3 equivalents) was added, and the mixture was stirred at room temperature for 1 hour. To the vessel was added MP-CNBH₃ resin (137 mg, 3 equivalents. 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40 mL/minute (0-0.5 minutes 5% A, 0.5-8.0 minutes linear gradient 5-80% A, 8.0-8.1 minutes linear gradient 80-100% A, 8.1-9.0 minutes 100% A, 9.0-9.1 minutes linear gradient 100-5% A, 9.1-10.0 minutes 5% A) to afford the title compound (8.2 mg, 16% yield). ¹H NMR (400 MHz, DMSO-d₆) δ ppm 8.86-8.69 (m, 2H), 6.26-6.21 (m, 1H), 4.52-4.18 (m, 4H), 4.13 (s, 2H), 3.79-3.75 (m, 1H), 3.12 (dd, J=43.2, 5.9 Hz, 3H), 2.79 (dd, J=16.9, 6.1 Hz, 1H), 0.85-0.69 (m, 4H); MS (APCI+) m/z 404.2 [M+H]⁺.

### Example 64: 5-[(3S)-5-fluoro-7-hydroxy-3-{[2-(oxolan-3-yl)ethyl]amino}-3,4-dihydro-2H-1-benzopyran-6-yl]-1λ⁶,2,5-thiadiazolidine-1,1,3-trione (Compound 382)

The product of Example 81 (43.1 mg, 0.10 mmol, 1 equivalent) was dissolved in 1.0 mL of acetic acid/sodium acetate buffer in methanol (adjusted to pH=4.5). 2-(Tetrahydrofuran-3-yl)acetaldehyde (0.6 M in methanol, 216 μL, 0.13 mmol, 1.3 equivalents) was added, and the mixture was stirred at room temperature for 1 hour. To the vessel was added MP-CNBH; resin (137 mg, 3 equivalents, 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40 mL/minute (0-0.5 minutes 5% A, 0.5-8.0 minutes linear gradient 5-80% A, 8.0-8.1 minutes linear gradient 80-100% A, 8.1-9.0 minutes 100% A, 9.0-9.1 minutes linear gradient 100-5% A, 9.1-10.0 minutes 5% A) to afford the title compound (6.7 mg, 13% yield). ¹H NMR (400 MHZ, DMSO-d₆) δ ppm 9.90 (s, 1H), 8.85-8.58 (m, 2H), 6.27-6.22 (m, 1H), 4.35-4.27 (m, 1H), 4.25-4.17 (m, 1H), 4.10 (s, 2H), 3.81-3.70 (m, 3H), 3.66-3.60 (m, 2H), 3.31-3.26 (m, 1H), 3.11-3.01 (m, 2H), 2.81 (dd, J=16.4, 4.4 Hz, 1H), 2.24-2.12 (m, 1H), 2.08-1.95 (m, 1H), 1.72-1.61 (m, 2H), 1.55-1.39 (m, 1H); MS (APCI+) m/z 416.2 [M+H]⁺.

### Example 65: 5-[(3S)-3-({[(1RS,5SR)-bicyclo[3.1.0]hexan-6-yl]methyl}amino)-5-fluoro-7-hydroxy-3,4-dihydro-2H-1-benzopyran-6-yl]-1λ⁶,2,5-thiadiazolidine-1,1,3-trione (Compound 383)

The product of Example 81 (43.1 mg, 0.10 mmol, 1 equivalent) was dissolved in 1.0 mL of acetic acid/sodium acetate buffer in methanol (adjusted to pH=4.5). Bicyclo[3.1.0]hexane-6-carbaldehyde (0.6 M in methanol, 216 μL, 0.13 mmol, 1.3 equivalents) was added, and the mixture was stirred at room temperature for 1 hour. To the vessel was added MP-CNBH₃ resin (137 mg, 3 equivalents, 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40 mL/minute (0-0.5 minutes 5% A, 0.5-8.0 minutes linear gradient 5-80% A, 8.0-8.1 minutes linear gradient 80-100% A, 8.1-9.0 minutes 100% A, 9.0-9.1 minutes linear gradient 100-5% A, 9.1-10.0 minutes 5% A) to afford the title compound (8.2 mg, 16% yield). ¹H NMR (400 MHZ, DMSO-d₆) δ ppm 8.72-8.64 (m, 1H), 6.23 (s, 1H), 4.31-4.17 (m, 2H), 4.07 (s, 2H), 3.77-3.73 (m, 1H), 3.01 (dq, J=34.4, 6.0 Hz, 3H), 2.79 (dd, J=17.0, 4.9 Hz, 1H), 1.78-1.48 (m, 5H), 1.37-1.28 (m, 2H), 1.15-0.98 (m, 1H), 0.90-0.81 (m, 1H); MS (APCI+) m/z 412.2 [M+H]⁺.

### Example 66; tert-butyl 4-({[(3S)-5-fluoro-7-hydroxy-6-(1,1,4-trioxo-1λ⁶,2,5-thiadiazolidin-2-yl)-3,4-dihydro-2H-1-benzopyran-3-yl]amino}methyl) piperidine-1-carboxylate (Compound 384)

The product of Example 81 (43.1 mg, 0.10 mmol, 1 equivalent) was dissolved in 1.0 mL of acetic acid/sodium acetate buffer in methanol (adjusted to pH=4.5), tert-Butyl 4-formylpiperidine-1-carboxylate (0.6 M in methanol, 250 μL, 0.15 mmol, 1.5 equivalents) was added, and the mixture was stirred at room temperature for 1 hour. To the vessel was added MP-CNBH₃ resin (137 mg, 3 equivalents. 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40) mL/minute (0-0.5 minutes 5% A, 0.5-8.0 minutes linear gradient 5-80% A, 8.0-8.1 minutes linear gradient 80-100% A, 8.1-9.0 minutes 100% A, 9.0-9.1 minutes linear gradient 100-5% A. 9.1-10.0 minutes 5% A) to afford the title compound (12.6 mg, 20% yield). ¹H NMR (500 MHz, DMSO-d₆) δ ppm 9.93-9.59 (m, 1H), 8.64 (s, 1H), 8.56 (s, 1H), 6.23 (d, J=1.6 Hz, 1H), 4.32-4.20 (m, 2H), 4.03 (s, 2H), 3.93 (d, J=13.1 Hz, 2H), 3.75 (s, 1H), 3.11-2.98 (m, 3H), 2.85-2.66 (m, 3H), 1.96-1.80 (m, 1H), 1.74 (d, J=12.6 Hz, 2H), 1.39 (s, 9H), 1.13-1.02 (m, 2H); MS (APCI+) m/z 515.2 [M+H]⁺.

### Example 67: 5-[(3S)-5-fluoro-7-hydroxy-3-{[(3-phenylcyclobutyl)methyl]amino}-3,4-dihydro-2H-1-benzopyran-6-yl]-1λ⁶,2,5-thiadiazolidine-1,1,3-trione (Compound 385)

The product of Example 81 (43.1 mg, 0.10 mmol, 1 equivalent) was dissolved in 1.0 mL of acetic acid/sodium acetate buffer in methanol (adjusted to pH=4.5). 3-Phenylcyclobutane-1-carbaldehyde (0.6 M in methanol, 250 μL, 0.15 mmol, 1.5 equivalents) was added, and the mixture was stirred at room temperature for 1 hour. To the vessel was added MP-CNBH₃ resin (137 mg, 3 equivalents. 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40) mL/minute (0-0.5 minutes 5% A, 0.5-8.0 minutes linear gradient 5-80% A, 8.0-8.1 minutes linear gradient 80-100% A, 8.1-9.0 minutes 100% A, 9.0-9.1 minutes linear gradient 100-5% A. 9.1-10.0 minutes 5% A) to afford the title compound (6.7 mg, 12% yield). ¹H NMR (400 MHz, DMSO-d₆) δ ppm 8.62 (s, 1H), 7.37-7.14 (m, 5H), 6.24 (d, J=1.8 Hz, 1H), 4.37-4.19 (m, 2H), 4.09 (s, 2H), 3.75-3.71 (m, 1H), 3.22-3.18 (m, 1H), 3.12-3.02 (m, 1H), 2.86-2.76 (m, 1H), 2.63-2.52 (m, 2H), 2.50-2.43 (m, 3H), 2.27 (t, J=7.7 Hz, 1H), 1.96-1.82 (m, 1H); MS (APCI+) m/z 462.2 [M+H]⁺.

### Example 68: 5-{(3S)-5-fluoro-7-hydroxy-3-[(3-phenylpropyl)amino]-3,4-dihydro-2H-1-benzopyran-6-yl}-1λ⁶,2,5-thiadiazolidine-1,1,3-trione (Compound 386)

Example 81 (43.1 mg, 0.10 mmol, 1 equivalent) was dissolved in 1.0 mL of acetic acid/sodium acetate buffer in methanol (adjusted to pH=4.5). 3-Phenylpropanal (0.6 M in methanol, 250 μL, 0.15 mmol, 1.5 equivalents) was added, and the mixture was stirred at room temperature for 1 hour. To the vessel was added MP-CNBH₃ resin (137 mg, 3 equivalents. 2.19 mmol/g loading), and the suspension was stirred at room temperature for 1 hour. The mixture was filtered, and the filtrate was purified using HPLC on a Phenomenex® Luna® C8(2) 5 μm 100 Å AXIA™ column (50 mm×30 mm). A gradient of methanol (A) and 0.1% trifluoroacetic acid in water (B) was used, at a flow rate of 40 mL/minute (0-0.5 minutes 5% A, 0.5-8.0) minutes linear gradient 5-80% A, 8.0-8.1 minutes linear gradient 80-100% A, 8.1-9.0 minutes 100% A. 9.0-9.1 minutes linear gradient 100-5% A, 9.1-10.0 minutes 5% A) to afford the title compound (3.4 mg, 6% yield). ¹H NMR (400 MHz, DMSO-d₆) δ ppm 9.86 (s, 1H), 8.67 (s, 1H), 7.36-7.27 (m, 2H), 7.27-7.17 (m, 3H), 6.23 (d, J=1.7 Hz, 1H), 4.34-4.25 (m, 1H), 4.23-4.15 (m, 1H), 4.09 (s, 2H), 3.81-3.76 (m, 1H), 3.11-3.00 (m, 3H), 2.79 (dd, J=17.2, 4.1 Hz, 1H), 2.66 (t, J=7.7 Hz, 2H), 1.91 (p, J=7.8 Hz, 2H); MS (APCI+) m/z 436.2 [M+H]⁺.

### Example 69: 5-[8-fluoro-6-hydroxy-2-(4-methylpentyl)-1-oxo-1,2,3,4-tetrahydroisoquinolin-7-yl]-1λ⁶,2,5-thiadiazolidine-1,1,3-trione (Compound 387)

### Example 69A: 5-[6-(benzyloxy)-8-fluoro-1-oxo-1,2,3,4-tetrahydroisoquinolin-7-yl]-1λ⁶,2,5-thiadiazolidine-1,1,3-trione, ammonium salt

Sodium chlorite (0.105 g, 0.929 mmol) was added to a solution of the product of Example 3H (0.300 g, 0.610 mmol) in tetrahydrofuran (2 mL) and water (2 mL). The mixture was brought to 55° C. and stirred for 1 hour. Additional sodium chlorite (0.060 g, 0.531 mmol) was added, and the mixture was stirred at 65° C. for another 3 hours. After cooling to room temperature, a saturated aqueous solution of sodium thiosulfate (1 mL) and diatomaceous earth were added. The mixture was then concentrated in vacuo. The crude residue was subjected to column chromatography (Büchi Reveleris C18, dry load with diatomaceous earth, 5-70% methanol in 10 mM ammonium bicarbonate) to afford the title compound as an ammonium salt (0.200 g, 0.450 mmol, 74% yield). ¹H NMR (400 MHz, DMSO-d₆) δ ppm 7.78 (t, J=3.2 Hz, 1H), 7.53-7.48 (m, 2H), 7.39-7.28 (m, 3H), 7.08 (t, J=50.8 Hz, 4H), 6.92 (s, 1H), 5.21 (s. 2H), 3.94 (s, 2H), 3.28 (td, J=6.3, 3.1 Hz, 2H), 2.83 (t, J=6.3 Hz, 2H); MS (ESI⁺) m/z 406 [M+H]⁺.

### Example 69B: 5-[6-(benzyloxy)-8-fluoro-2-(4-methylpentyl)-1-oxo-1,2,3,4-tetrahydroisoquinolin-7-yl]-1λ⁶,2,5-thiadiazolidine-1,1,3-trione, ammonium salt

Sodium hydride, 60 weight % (0.027 g, 0.675 mmol) was added to a suspension of the product of Example 69A (0.110 g, 0.260 mmol) in N,N-dimethylformamide (2.5 mL) at 0° C. The resulting mixture was stirred at 0° C. for 15 minutes before the addition of 1-bromo-4-methylpentane (0.040 mL, 0.275 mmol). The mixture was stirred at 0° C. for 4 hours and at room temperature for 18 hours. The mixture was subjected directly to column chromatography (Büchi Reveleris® C18, 5-70% methanol in 10 mM ammonium bicarbonate) to afford the title compound as a partial ammonium salt (0.112 g, 0.199 mmol, 76% yield, 90% purity). ¹H NMR (400 MHZ, DMSO-d₆) δ ppm 7.53-7.47 (m, 2H), 7.39-7.27 (m, 3H), 7.09 (br s, 4H), 6.90 (s, 1H), 5.21 (s, 2H), 3.93 (s, 2H), 3.46 (t, J=6.3 Hz, 2H), 3.40 (t, J=7.3 Hz, 2H), 2.88 (t, J=6.3 Hz, 2H), 1.61-1.46 (m, 3H), 1.19-1.11 (m, 2H), 0.86 (d, J=6.6 Hz, 6H); MS (ESI⁺) m/z 490 [M+H]⁺.

### Example 69C: 5-[8-fluoro-6-hydroxy-2-(4-methylpentyl)-1-oxo-1,2,3,4-tetrahydroisoquinolin-7-yl]-1λ⁶,2,5-thiadiazolidine-1,1,3-trione

10% Pd/C (15 mg) was added to a suspension of the product of Example 69B (0.052 g, 0.096 mmol) in ethanol (2 mL) and dioxane (2 mL) and the resulting mixture was hydrogenated at 4 bars for 1 hour. The mixture was filtered through a pad of diatomaceous earth which was washed with methanol (25 mL). The filtrate was concentrated in vacuo. The crude residue was purified by preparative HPLC (Waters, 0.1% ammonium hydroxide, Waters XBridge BEH column C18, 5 μm, 30×100 mm, 15-100% acetonitrile in water, flow rate of 40 mL/minute) to afford the title compound as an ammonium salt (0.027 g, 0.062 mmol, 64% yield). ¹H NMR (400 MHz, DMSO-d₆) δ ppm 7.25 (br s, 5H), 6.54 (s, 1H), 3.91 (s, 2H), 3.46-3.34 (m, 4H), 2.81 (t, J=6.3 Hz, 2H), 1.60-1.44 (m, 3H), 1.19-1.10 (m, 2H), 0.86 (d, J=6.6 Hz, 6H); MS (ESI⁺) m/z 400 [M+H]⁺.

### Example 70: 5-(8-fluoro-6-hydroxy-1-oxo-1,2,3,4-tetrahydroisoquinolin-7-yl)-1λ⁶,2,5-thiadiazolidine-1,1,3-trione (Compound 388)

10% Pd/C (16 mg) was added to a suspension of the product of Example 69A (0.060 g, 0.148 mmol) in ethanol (2 mL) and dioxane (2 mL). The resulting suspension was allowed to stir under hydrogen (4 bars) for 20 hours. The mixture was filtered through a glass fiber filter which was washed with methanol (20 mL) and water (10 mL). The combined filtrates were concentrated in vacuo. The crude residue was purified by preparative HPLC (Waters, Acidic (0.1% Formic acid). Waters XSelect® CSH column C18, 5 μm, 30×100 mm, 10-30% acetonitrile in water, flow rate of 40 mL/minute) to afford the title compound (0.0308 g, 0.093 mmol, 63% yield). ¹H NMR (400 MHz, DMSO-d₆) δ ppm 10.90 (s, 1H), 7.78 (t, J=3.1 Hz, 1H), 6.63 (s, 1H), 4.32 (s, 2H), 3.26 (td, J=6.4, 3.0 Hz, 2H), 2.80 (t, J=6.3 Hz, 2H); MS (ESI⁺) m/z 316 [M+H]⁺.

### Example 71: 5-[7-(aminomethyl)-1-fluoro-3-hydroxy-5,6,7,8-tetrahydronaphthalen-2-yl]-1λ⁶,2,5-thiadiazolidine-1,1,3-trione (Compound 389)

To a solution of the product of Example 73 (15 mg, 0.035 mmol) in dichloromethane (2 mL) was added 2,2,2-trifluoroacetic acid (200 μL, 2.60 mmol) and the mixture stirred at ambient temperature for 1 hour and 15 minutes. The reaction mixture was concentrated under reduced pressure and the residue was azeotroped with toluene (3×2 mL). The resultant residue was triturated with acetonitrile to give the title compound as a trifluoroacetic acid salt (8.1 mg, 0.018 mmol, 52.3% yield). ¹H NMR (400 MHz, DMSO-d₆) δ ppm 9.03 (s, 1H), 7.78 (s, 3H), 6.45 (d, J=1.5 Hz, 1H), 3.94 (d, J=1.1 Hz, 2H), 2.94-2.58 (m, 5H), 2.21 (dd, J=16.5, 10.5 Hz, 1H), 1.93-1.86 (m, 2H), 1.42-1.27 (m, 1H); MS (APCI+) m/z 330 [M+H]⁺.

### Example 72: 5-(1-fluoro-3-hydroxy-7-{[(3-methylbutyl)amino]methyl}-5,6,7,8-tetrahydronaphthalen-2-yl)-1λ⁶,2,5-thiadiazolidine-1,1,3-trione (Compound 390)

### Example 72A: 5-[3-(benzyloxy)-1-fluoro-7-{[(3-methylbutyl)amino]methyl}-5,6,7,8-tetrahydronaphthalen-2-yl]-1λ⁶,2,5-thiadiazolidine-1,1,3-trione

To a suspension of the product of Example 15J (41.1 mg, 0.077 mmol) in dichloromethane (1 mL) and ethanol (2 mL) was added triethylamine (0.043 mL, 0.308 mmol). The reaction mixture was allowed to stir at ambient temperature for 5 minutes, after which 3-methylbutanal (0.042 mL, 0.385 mmol) was added and stirring was continued for 2 hours. Sodium tetrahydroborate (23.30 mg, 0.616 mmol) was then added and the reaction mixture was stirred for an additional 1 hour. The reaction was quenched with aqueous 1 M HCl (0.5 mL) and the mixture was concentrated under reduced pressure with diatomaceous earth for dry loading. The product was purified by reverse phase column chromatography (30 g Biotage® Sfär C18 Duo 100 Å 30 μm column. 10 to 100% methanol in water | buffered with 0.025 M aqueous ammonium bicarbonate, adjusted to pH 7 with CO₂ (s)], flow rate=25 mL/minute) to give the title compound (24.9 mg, 0.051 mmol, 66.0% yield). MS (APCI⁺) m/z 490 [M+H]⁺.

### Example 72B: 5-(1-fluoro-3-hydroxy-7-{[(3-methylbutyl)amino]methyl}-5,6,7,8-tetrahydronaphthalen-2-yl)-1λ⁶,2,5-thiadiazolidine-1,1,3-trione

The product of Example 72A (24.9 mg, 0.051 mmol), ammonium formate. (25.7 mg, 0.407 mmol), and 10% Pd/C (5.41 mg, 5.09 μmol) in ethanol (3 mL) was heated to 50° C. for 2 hours, and then heated to 65° C. for 2 hours. The reaction mixture was cooled to ambient temperature, filtered over a pad of diatomaceous earth, and the pad was rinsed with methanol. The filtrate was concentrated under reduced pressure and purified by reverse phase column chromatography (30 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=25 mL/minute) to give the title compound (4 mg, 10.01 μmol, 19.69% yield). ¹H NMR (500 MHZ, DMSO-d₆) δ ppm 9.01 (s, 1H), 8.16 (s, 2H), 6.44 (s, 1H), 3.95-3.91 (m, 2H), 2.95-2.81 (m, 5H), 2.77-2.62 (m, 2H), 2.25-2.16 (m, 1H), 2.01-1.98 (m, 1H), 1.89 (d, J=12.8 Hz, 1H), 1.63 (dt, J=13.4, 7.0 Hz, 1H), 1.53-1.44 (m, 2H), 1.40-1.34 (m, 1H), 0.90 (d, J=6.6 Hz, 6H); MS (APCI+) m/z 400 [M+H]⁺.

### Example 73: tert-butyl {[8-fluoro-6-hydroxy-7-(1,1,4-trioxo-1λ⁶,2,5-thiadiazolidin-2-yl)-1,2,3,4-tetrahydronaphthalen-2-yl]methyl}carbamate (Compound 391)

The product of Example 151 (60 mg, 0.115 mmol), ammonium formate (58 mg, 0.920) mmol), and 10% Pd/C (12 mg, 0.011 mmol) in ethanol (3 mL) was heated to 50° C. for 2 hours, and then heated to 65° C. for 2 hours. The reaction mixture was cooled to ambient temperature, filtered over a pad of diatomaceous earth, and the pad was rinsed with methanol. The filtrate was concentrated under reduced pressure and the residue was purified by reverse phase column chromatography (30 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=25 mL/minute) to give the title compound as the ammonium salt (35.3 mg, 0.079 mmol, 68.5% yield). ¹H NMR (400 MHZ, DMSO-d₆) δ ppm 8.94 (s, 1H), 7.08 (s, 4H), 6.95 (t, J=5.7 Hz, 1H), 6.41 (s, 1H), 3.92 (s, 2H), 3.00-2.87 (m, 2H), 2.77-2.53 (m, 3H), 2.06 (dd, J=16.7, 10.4 Hz, 1H), 1.83-1.67 (m, 2H), 1.39 (s, 9H), 1.28-1.19 (m, 1H); MS (ESI⁻) m/z 428 [M−H]⁻.

### Example 74; tert-butyl [(2R)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate (Compound 392)

### Example 74A: (2R)-2-[(tert-butoxycarbonyl)amino]-4-{[tert-butyl(dimethyl)silyl]oxy}butanoic acid

To a stirred solution of (2R)-2-[(tert-butoxy carbonyl)amino]-4-hydroxy butanoic acid (10.55 g, 48.1 mmol), 1H-imidazole (6.55 g, 96 mmol) and N,N-dimethylformamide (96 mL) was added tert-butylchlorodimethylsilane (7.25 g, 48.1 mmol) in one portion. After stirring overnight, the reaction was concentrated. The residue was taken up in tert-butyl methyl ether and washed with aqueous 1 M HCl and brine, dried over Na₂SO₄, filtered, and the filtrate was concentrated to give the title compound (16.6 g, 49.6 mmol, 103% yield), which was carried on to the next step without further purification (assumed 100% yield). MS (APCI⁺) m/z 322 [M−H]⁻.

### Example 74B: tert-butyl [(2R)-4-{[tert-butyl(dimethyl)silyl]oxy}-1-hydroxybutan-2-yl]carbamate

To a stirred solution of the product of Example 74A (12.17 g, 36.5 mmol) in tetrahydrofuran (182 mL) at 0° C. was added 4-methylmorpholine (4.21 mL, 38.3 mmol) and isobutyl carbonochloridate (5.11 mL, 38.3 mmol). After 30 minutes, the reaction mixture was filtered through a plug of Celite®, washing the filter cake with additional tetrahydrofuran. A solution of sodium borohydride (2.76 g, 73.0 mmol) in water (45 mL) was added to a stirred solution of the combined filtrates. After 1 hour the reaction was quenched by addition of aqueous 1 M HCl and partially concentrated. The mixture was transferred to a separatory funnel and extracted three times with ethyl acetate. The combined organic fractions were washed with brine, dried over Na₂SO₄, filtered, and the filtrate was concentrated. The residue was loaded onto a 220 g Teledyne ISCO silica gel column and eluted using a gradient of 12-100% ethyl acetate in heptanes, yielding the title compound (12.0 g, 37.6 mmol, 103% yield). MS (APCI⁺) m/z 320 [M+H]⁺.

### Example 740; tert-butyl (4R)-4-(2-{[tert-butyl(dimethyl)silyl]oxy}ethyl)-2-oxo-1,2λ⁴,3-oxathiazolidine-3-carboxylate

A solution of thionyl chloride (3.29 mL, 45.1 mmol) in dichloromethane (16 mL) was added slowly to a solution of imidazole (10.23 g, 150 mmol) and triethylamine (15.70 mL, 113 mmol) in dichloromethane (114 mL) at −40° C. The mixture was stirred for 15 minutes at −40° C., during which time a thick slurry formed. A solution of the product of Example 74B (12.0 g, 37.6 mmol) in dichloromethane (26 mL) was added over a 30 minute period, maintaining the cooling bath at −40° C. The reaction mixture was allowed to warm to ambient temperature and stirred overnight. The mixture was slowly diluted with a saturated aqueous solution of NaHCO₃ and transferred to a separatory funnel. The layers were separated, and the organic layer was washed with brine, dried over Na₂SO₄, filtered, and the filtrate was concentrated. To remove residual imidazole, the residue was taken up in tert-butyl methyl ether and washed three times with water, before drying over Na₂SO₄, and concentrating to give the title compound (13.8 g, 37.8 mmol, 101% yield) which was carried on to the next step without further purification (assumed 100% yield). ¹H NMR (600.4 MHZ, CDCl₃) δ ppm 4.86-4.76 (m, 2H), 3.92 (tdd, J=9.4, 7.2, 2.9 Hz, 1H), 3.70 (dt, J=10.7, 4.6 Hz, 1H), 3.60 (ddd, J=10.7, 9.4, 3.6 Hz, 1H), 1.83-1.74 (m, 1H), 1.47 (s, 9H), 0.84 (d, J=1.1 Hz, 9H), 0.00 (d, J=1.7 Hz, 6H); MS (APCI+) m/z 366 [M+H]⁺.

### Example 74D: tert-butyl (4R)-4-(2-{[tert-butyl(dimethyl)silyl]oxy}ethyl)-2,2-dioxo-1,2λ⁶,3-oxathiazolidine-3-carboxylate

To a solution of the product of Example 74C (13.8 g, 37.8 mmol) in acetonitrile (101 mL) and water (25.2 mL) was added ruthenium(III) chloride hydrate (0.078 g, 0.378 mmol) and sodium periodate (8.88 g, 41.5 mmol). The reaction mixture was stirred for 3 minutes at 23° C. before dilution with ethyl acetate (200 mL). The diluted mixture was filtered through a polyethylene frit packed with diatomaceous earth and the filter cake was washed with ethyl acetate three times. The combined filtrates were transferred to a separatory funnel and washed with saturated aqueous sodium thiosulfate solution (150 mL) and brine, dried over MgSO₄, filtered, and the filtrate was concentrated. The residue was loaded onto a 220 g Teledyne ISCO silica gel column and eluted using a 2-2-% gradient of ethyl acetate in heptanes to yield the title compound (10.4 g, 27.2 mmol, 72%). ¹H NMR (500.2 MHZ, CDCl₃) δ ppm 4.69-4.63 (m, 2H), 4.39 (ddt, J=9.0, 5.5, 3.3 Hz, 1H), 3.81 (ddd, J=10.8, 5.5, 4.4 Hz, 1H), 3.74 (ddd, J=10.8, 8.5, 3.8 Hz, 1H), 2.19-2.11 (m, 1H), 2.09-1.98 (m, 1H), 1.56 (s, 9H), 0.89 (s, 9H), 0.06 (s, 3H), 0.06 (s, 3H); MS (APCI+) m/z 399 [M+NH₄]⁺.

### Example 74E: tert-butyl [(2S)-1-[4-(benzyloxy)-6-bromo-2-fluoro-3-(2,2,2-trifluoroacetamido)phenyl]-4-{[tert-butyl(dimethyl)silyl]oxy}butan-2-yl]carbamate

A 250 mL round bottom flask was charged with tetrahydrofuran (67.5 mL), cooled to −78° C., and charged with diisopropylamine (8.46 mL, 59.4 mmol), followed by dropwise addition of n-butyllithium (2.50) M, 22.68 mL, 56.7 mmol). After stirring for 15 minutes, a solution of the product from Example 1C in tetrahydrofuran (33.7 mL) was added dropwise over 20 minutes and the resultant mixture was stirred for 30 minutes. Subsequently, a solution of the product from Example 74D (10.3 g, 27.0 mmol) in tetrahydrofuran (33.7 mL) was added dropwise and the resultant mixture was stirred for an additional 30 minutes at −78° C. The reaction was quenched at −78° C. by addition of aqueous 1 M HCl (67.5 mL, 135 mmol) and the mixture was allowed to warm to ambient temperature. The mixture was diluted with ethyl acetate and transferred to a separatory funnel, where it was washed with water and brine, dried over Na₂SO₄, filtered, and the filtrate was concentrated. The residue was loaded onto a 220 g Teledyne ISCO silica gel column and eluted with a gradient of 2-20% ethyl acetate in heptanes to give the title compound (8.4 g, 12.1 mmol, 45% yield). ¹H NMR (499.6 MHZ, CDCl₃) δ ppm 7.58 (s, 1H), 7.42-7.33 (m, 5H), 7.05 (s, 1H), 5.21-5.01 (m, 2H), 4.10-4.04 (m, 1H), 3.84 (s, 1H), 3.76-3.70 (m, 1H), 3.09-2.98 (m, 1H), 2.95-2.85 (m, 1H), 1.90-1.80 (m, 1H), 1.66 (d, J=5.4 Hz, 1H), 1.31 (s, 9H), 0.91 (s, 9H), 0.07 (d, J=5.3 Hz, 6H); MS (APCI+) m/z 693 [M+H]⁺.

### Example 293F; methyl [{6-(benzyloxy)-4-bromo-3-[(2S)-2-[(tert-butoxycarbonyl)amino]-4-{[tert-butyl(dimethyl)silyl]oxy}butyl]-2-fluorophenyl}(trifluoroacetyl)amino]acetate

A 100 mL round bottom flask was charged with the product from Example 74E (8.4 g, 12.11 mmol), acetone (60.6 mL), potassium carbonate (5.02 g, 36.3 mmol), potassium iodide (1.005 g, 6.06 mmol), and methyl bromoacetate (1.228 mL, 13.32 mmol) and the mixture was vigorously stirred under N₂ at ambient temperature. After 4 hours, the reaction mixture was diluted with ethyl acetate and transferred to a separatory funnel. The solution was washed with water, and the aqueous layer was back extracted with ethyl acetate. The combined organic layers were washed with brine and concentrated. The residue was loaded onto a 220 g Teledyne ISCO silica gel column and eluted with a gradient of 10-80% ethyl acetate in heptanes to give the title compound (8.5 g, 11.1 mmol, 92% yield). MS (APCI⁺) m/z 766 [M+H]⁺.

### Example 74G; methyl {[6-(benzyloxy)-4-bromo-3-{(2S)-2-[(tert-butoxycarbonyl)amino]-4-hydroxybutyl}-2-fluorophenyl](trifluoroacetyl)amino}acetate

A 50 mL flask was charged with the product from Example 74F (0.800 g, 1.045 mmol) acetic acid (7.8 mL), tetrahydrofuran (2.6 mL), and water (2.6 mL) and the mixture was rapidly stirred at 40° C. After two hours, the mixture was cooled to ambient temperature and carefully diluted with ethyl acetate and saturated aqueous sodium bicarbonate. The mixture was transferred to a separatory funnel and the organic layer was separated. The aqueous layer was extracted with ethyl acetate three times, and the combined organic layers were dried over Na₂SO₄, filtered, and the filtrate was concentrated. The residue was loaded onto a 40 g Teledyne ISCO silica gel column and eluted with a gradient of 10-80% ethyl acetate in heptanes to give the title compound (520 mg, 0.798 mmol, 76% yield). MS (APCI⁺) m/z 651 [M+H]⁺.

### Example 74H: methyl {[6-(benzyloxy)-4-bromo-3-{(2S)-2-[(tert-butoxycarbonyl)amino]-4-oxobutyl}-2-fluorophenyl](trifluoroacetyl)amino}acetate

A 25 mL round bottom flask was charged with the product of Example 74G (1.29 g, 1.980 mmol) and dichloromethane (19.88 mL). After cooling to 0° C., 1,1,1-tris (acetyloxy)-1,1-dihydro-1,2-benziodoxol-3-(1H)-one (1.680 g, 3.96 mmol, Dess-Martin periodinane) in dichloromethane (3.98 mL) was added dropwise. After 15 minutes, the reaction mixture was diluted with water and ethyl acetate and filtered through a polyethylene frit packed with diatomaceous earth. The filtrate was transferred to a separatory funnel, and the layers were separated. The organic layer was washed with brine, dried over Na₂SO₄, filtered, and the filtrate was concentrated. The residue was loaded onto an 80 g Teledyne ISCO silica gel column that was eluted with a 20-60% gradient of ethyl acetate in heptanes to yield the title compound (1.14 g, 1.755 mmol, 89% yield). MS (APCI⁺) m/z 666 [M+NH₄]⁺.

### Example 74I: methyl {[6-(benzyloxy)-4-bromo-3-{(2R)-2-[(tert-butoxycarbonyl)amino]pent-4-en-1-yl}-2-fluorophenyl](trifluoroacetyl)amino}acetate

A heat dried 50 mL round bottom flask was charged with methyltriphenylphosphonium bromide (0.658 g, 1.843 mmol) and toluene (8.78 mL) and the mixture was cooled to 0° C. in a dry-ice acetone bath under N₂. A solution of sodium bis(trimethylsilyl)amide (0.6 M in toluene, 2.93 mL, 1.755 mmol, NaHMDS) was added dropwise and the resulting solution was stirred for 25 minutes at 0° C. before cooling to −78° C. A solution of the product of Example 74H (845 mg, 1.305 mmol, 74.3% yield) in toluene (1.756 mL) was added in one portion and stirred for 30 minutes before allowing the reaction mixture to warm to ambient temperature. The reaction was quenched with a saturated aqueous solution of NH₄Cl, and the mixture was transferred to a separatory funnel and extracted three times with ethyl acetate. The combined organic layers were washed with brine, dried over MgSO₄, filtered, and the filtrate was concentrated onto 10 g of SiO₂. The residue was loaded onto an 80 g Teledyne ISCO silica gel column and was eluted with a gradient of 10-50% ethyl acetate in heptanes to yield the title compound (845 mg, 1.305 mmol, 74.3% yield). MS (APCI⁺) m/z 648 [M+H]⁺.

### Example 74J: methyl [{(7S)-3-(benzyloxy)-7-[(tert-butoxycarbonyl)amino]-1-fluoro-5-methyl-7,8-dihydronaphthalen-2-yl}(trifluoroacetyl)amino]acetate

A 50 mL round bottom flask was charged with the product of Example 741 (0.405 g, 0.626 mmol), 1,4-dioxane (12.51 mL), palladium(II) acetate (0.014 g, 0.063 mmol), triphenylphosphine (0.033 g, 0.125 mmol), and potassium carbonate (0.519 g, 3.75 mmol). The reaction mixture was sparged for 30 minutes with N₂ and heated to 90° C. on a preheated reaction block. After 3 hours, the reaction mixture was cooled to ambient temperature, filtered through a polyethylene frit packed with diatomaceous earth, and concentrated onto 5 g of SiO₂. The residue was dry loaded onto a 24 g Teledyne ISCO silica gel column and eluted with a 5-20% gradient of ethyl acetate in heptanes to yield the title compound (220 mg, 0.388 mmol, 62.1% yield). MS (APCI⁺) m/z 584 [M+NH₄]⁺.

### Example 74K: methyl ({(7S)-3-(benzyloxy)-7-[(tert-butoxycarbonyl)amino]-1-fluoro-5-methyl-7,8-dihydronaphthalen-2-yl}amino)acetate

A 20 mL screw top vial was charged with the product of Example 74J (220 mg, 0.388 mmol) and sodium methoxide (0.5 M in methanol, 2330 μL, 1.165 mmol). The vial was heated to 60° C. on a preheated reaction block. After 1 hour, the reaction was quenched by addition of saturated aqueous solution of NH₄Cl and water (1:1) and the mixture was transferred to a separatory funnel. The layers were separated, and the aqueous layer was extracted three times with ethyl acetate. The combined organic layers were washed with brine, dried over Na₂SO₄, filtered, and the filtrate was concentrated to afford the title compound which was used without further purification in the next step. MS (APCI⁺) m/z 471 [M+H]⁺.

### Example 74L: methyl [{(7S)-3-(benzyloxy)-7-[(tert-butoxycarbonyl)amino]-1-fluoro-5-methyl-7,8-dihydronaphthalen-2-yl}({[(prop-2-en-1-yl)oxy]carbonyl}sulfamoyl)amino]acetate

A heat-dried 10 mL round bottom flask was charged with dichloromethane (1296 μL) and chlorosulfonyl isocyanate (50.7 μL, 0.583 mmol) and the mixture was cooled to −20° C. in a dry ice/acetone bath. Allyl alcohol (39.7 μL, 0.583 mmol) was added dropwise over a 5 minute period. After 30 minutes, a solution of the product of Example 74K (183 mg, 0.389 mmol) and triethylamine (136 μL, 0.9725 mmol) in dichloromethane (648 μL) was added via syringe. After 5 minutes, the reaction mixture was diluted with ethyl acetate and water and transferred to a separatory funnel. The layers were separated, and the aqueous layer was back extracted with ethyl acetate three times. The combined organic fractions were dried over Na₂SO₄, filtered, and the filtrate was concentrated onto 2 g of SiO₂. The residue was dry loaded onto a 24 g Teledyne ISCO silica gel column and eluted with a gradient of 25-95% ethyl acetate in heptanes to yield the title compound (187 mg, 0.295 mmol, 76% yield). MS (ESI⁺) m/z 651 [M+NH₄]⁺.

### Example 74M: tert-butyl [(2S)-6-(benzyloxy)-8-fluoro-4-methyl-7-(1,1,4-trioxo-1λ⁶,2,5-thiadiazolidin-2-yl)-1,2-dihydronaphthalen-2-yl]carbamate

A 1 dram vial was charged with the product of Example 74L (187 mg, 0.295 mmol), tetrakis(triphenylphosphine)palladium(0)) (5.9 mg, 5.11 μmol), and a solution of sodium methoxide (0.5 M in methanol, 1532 μL, 0.766 mmol). The vial was sparged with N₂ for 5 minutes before being placed in a preheated reaction block at 60° C. After 30 minutes, the reaction mixture was cooled to ambient temperature and quenched by addition of 4 M HCl in 1,4-dioxane (49 μL, 0.197 mmol). The mixture was transferred to a separatory funnel and diluted with brine and ethyl acetate. The layers were separated, and the aqueous layer was extracted with ethyl acetate three times. The combined organic layers were dried over Na₂SO₄ and filtered through a polyethylene frit packed with diatomaceous earth, washing the frit three times with ethyl acetate. The combined filtrate and washes were concentrated to give the title compound (143 mg, 0.276 mmol, 108% yield), which was used in the next step without further purification (assumed 100% yield). MS (APCI⁺) m/z 535 [M+NH₄]⁺.

### Example 74N; tert-butyl [(2R)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate

A 1 dram vial was charged with the product of Example 74M (153 mg, 0.295 mmol), ammonium formate (130 mg, 2.065 mmol), Pd/C (10 weight %, 94 mg, 0.0885 mmol), and ethanol (1475 μL). The vial was flushed with N₂, sealed, and heated to 60° C. After 30 minutes, the reaction mixture was cooled to ambient temperature and passed through a polyethylene frit packed with diatomaceous earth, washing the frit with ethanol twice. The combined washes and filtrate were concentrated, and the residue was passed through a small pad of SiO₂ in ethyl acetate/ethanol (4:1) and concentrated. The obtained residue (122 mg) was loaded onto a 12 g Teledyne ISCO silica gel column in ethyl acetate/ethanol and eluted with a gradient of methanol in ethyl acetate (5-100%) to yield the title compound (110 mg, 0.256 mmol 87% yield) as a mixture of cis and trans diastereomers (2:1). ¹H NMR (DMSO-d₆) δ ppm 9.01 (s, 1H), 6.96 (d, J=7.7 Hz, 1H), 6.61 (s, 1H), 3.97-3.89 (m, 2H), 3.56-3.53 (m, 1H), 2.86-2.79 (m, 2H), 2.32-2.24 (m, 1H), 2.01-1.94 (m, 1H), 1.40 (s, 9H), 1.24-1.20 (m, 3H); MS (APCI+) m/z 447 [M+NH₄]⁺.

### Example 75: 5-{(6R,7S)-1-fluoro-3,6-dihydroxy-7-[(3-methylbutyl)amino]-5,6,7,8-tetrahydronaphthalen-2-yl}-1λ⁶,2,5-thiadiazolidine-1,1,3-trione (Compound 393)

### Example 75A: tert-butyl [(2S)-1-[4-(benzyloxy)-6-ethenyl-2-fluoro-3-(2,2,2-trifluoroacetamido)phenyl]-3-{[tert-butyl(dimethyl)silyl]oxy}propan-2-yl]carbamate

To a mixture of the product of Example 8A (37 g, 49.0 mmol) in dioxane (400 mL) and water (40 mL) was added potassium trifluoro (vinyl) borate (39.4 g, 294 mmol), [1,1′-bis(diphenylphosphino)ferrocene]dichloropalladium(II) (3.59 g, 4.90 mmol) and K₂CO₃ (40.6 g, 294 mmol) in order. The mixture was heated to 100° C. for 12 hours under N₂. One additional reaction on 5 g scale was run as described above. The reaction mixtures were combined and filtered. The filtrate was poured into water (500 mL), and the mixture was extracted with ethyl acetate (3×500) mL). The combined organic layers were washed with brine (1000 mL), dried over Na₂SO₄, and concentrated under reduced pressure. The residue was purified by column chromatography on silica gel eluting with ethyl acetate in petroleum ether from 0% to 1% to give the title compound (34 g, 48.8 mmol, yield 88%, purity 90%), MS (ESI⁻) m/z 625 [M−H]⁻.

### Example 75B: tert-butyl {(2S)-1-[4-(benzyloxy)-6-ethenyl-2-fluoro-3-(2,2,2-trifluoroacetamido)phenyl]-3-hydroxypropan-2-yl}carbamate

To a solution of the product of Example 75A (26 g, 37.3 mmol, purity 90%) in tetrahydrofuran (90 mL) and water (90 mL) was added acetic acid (270 mL, 37.3 mmol) at 25° C. The mixture was stirred at 25° C. for 12 hours. One additional reaction on 8 g scale was run as described above. The reaction mixtures were poured into water (600 mL) and extracted with ethyl acetate (3×400 mL). The combined organic layers were washed with brine (1000 mL), dried over Na₂SO₄, and concentrated under reduced pressure. The residue was purified by column chromatography on silica gel eluted with ethyl acetate in petroleum ether from 10% to 30% to give the title compound (24 g, 42.1 mmol, yield 78%, purity 90%). ¹H NMR (400 MHZ, DMSO-d₆) δ ppm 1.31 (s, 9H), 2.60-2.87 (m, 2H), 3.26-3.31 (m, 2H), 3.46-3.57 (m, 1H), 4.72 (br t, J=5.44 Hz, 1H), 5.22 (s, 2H), 5.42 (br d, J=11.25 Hz, 1H), 5.87 (br d, J=17.24 Hz, 1H), 6.54 (br d, J=8.68 Hz, 1H), 7.13 (br dd, J=17.24, 11.13 Hz, 1H), 7.19 (s, 1H), 7.28-7.35 (m, 1H), 7.35-7.47 (m, 5H), 10.97 (s, 1H).

### Example 75C: tert-butyl {(2S)-1-[4-(benzyloxy)-6-ethenyl-2-fluoro-3-(2,2,2-trifluoroacetamido)phenyl]-3-oxopropan-2-yl}carbamate

To a solution of the product of Example 75B (18.5 g, 32.5 mmol, purity 90%) and triethylamine (19.72 g, 195 mmol) in dichloromethane (150 mL) and dimethyl sulfoxide (150 mL) was added a solution of pyridine sulfur trioxide (31.0 g, 195 mmol) in dimethyl sulfoxide (150 mL) dropwise at 0° C. The mixture was stirred at 0° C. for 1 hour. Two additional reactions on 200 mg scale were run as described above. The reaction mixtures were poured into saturated aqueous NaHCO₃ (400 mL), and the mixture was extracted with dichloromethane (3×400 mL). The combined organic phases were dried over Na₂SO₄ and concentrated under reduced pressure. The residue was purified by column chromatography on silica gel eluted with ethyl acetate in petroleum ether from 10% to 30% to give the title compound (18 g, 31.7 mmol, yield 67.8%, purity 90%). ¹H NMR (400 MHZ, DMSO-d₆) δ ppm 11.00 (s, 1H), 9.46 (s, 1H), 7.49-7.35 (m, 6H), 7.35-7.29 (m, 1H), 7.19 (s, 1H), 6.93 (dd, J=11.1, 17.0 Hz, 1H), 5.87 (d, J=17.8 Hz, 1H), 5.42 (d, J=11.4 Hz, 1H), 5.23 (s, 2H), 3.87-3.72 (m, 1H), 3.17-2.85 (m, 2H), 1.34 (s, 9H).

### Example 75D: tert-butyl {(2S)-1-[4-(benzyloxy)-6-ethenyl-2-fluoro-3-(2,2,2-trifluoroacetamido)phenyl]but-3-en-2-yl}carbamate

To a solution of methyltriphenylphosphonium bromide (11.34 g, 31.7 mmol) in tetrahydrofuran (100 mL) was added NaH (1.058 g, 26.4 mmol) in portions at 0° C. The mixture was stirred at 0° C. for 0.5 hour. Then a solution of the product of Example 75C (10 g, 17.63 mmol) in tetrahydrofuran (50 mL) was dropwise added into the above mixture at 0° C. The resulting mixture was stirred for 2 hours at 25° C. Two additional reactions on 2 g and 5 g scale, respectively, were run as described above. The reaction mixtures were slowly poured into saturated aqueous NH₄Cl (300 mL), and the resultant mixture was extracted with ethyl acetate (3×200 mL). The combined organic layers were washed with brine (300 mL), dried over Na₂SO₄, and concentrated under reduced pressure. The residue was purified by column chromatography on silica gel eluted with ethyl acetate in petroleum ether from 5% to 20% to give the title compound (8 g, 14.16 mmol, yield 47.4%, purity 90%). ¹H NMR (400 MHZ, DMSO-d₆) δ ppm 10.97 (br s, 1H), 7.50-7.27 (m, 5H), 7.19 (s, 1H), 7.12-6.96 (m, 2H), 5.87 (br d, J=17.1 Hz, 1H), 5.80-5.65 (m, 1H), 5.45 (br d, J=10.9 Hz, 1H), 5.22 (s, 2H), 4.99-4.85 (m, 2H), 4.13-3.98 (m, 1H), 2.80 (br s, 2H), 1.33 (br s, 9H).

### Example 75E: methyl {[6-(benzyloxy)-3-{(2S)-2-[(tert-butoxycarbonyl)amino]but-3-en-1-yl}-4-ethenyl-2-fluorophenyl](trifluoroacetyl)amino}acetate

To a solution of the product of Example 75D (7 g, 12.39 mmol, purity 90%) in N,N-dimethylformamide (70 mL) was added methyl 2-bromoacetate (2.84 g, 18.58 mmol) and K₂CO₃ (5.14 g, 37.2 mmol) at 25° C. The mixture was stirred at 60° C. for 3 hours. One additional reaction on 1 g scale was run as described above. The reaction mixtures were poured into water (200 mL), and the resultant mixture was then extracted with ethyl acetate (3×80 mL). The organic layer was washed with brine (200 mL), dried over Na₂SO₄, and concentrated under reduced pressure. The residue was purified by column chromatography on silica gel eluted with ethyl acetate in petroleum ether from 5% to 30% to give the title compound (8.4 g, 13.02 mmol, yield 92%, purity 90%). ¹H NMR (400 MHZ, DMSO-d₆) δ ppm 1.27-1.38 (m, 9H), 2.81 (br s, 2H), 3.61 (s, 3H), 3.97-4.12 (m, 2H), 4.15-4.26 (m, 1H), 4.50 (br d, J=16.76 Hz, 1H), 4.81-4.97 (m, 2H), 5.17-5.31 (m, 2H), 5.51 (br dd, J=11.13, 5.00 Hz, 1H), 5.60-5.81 (m, 1H), 5.95 (dd, J=17.20, 11.07 Hz, 1H), 6.92-7.14 (m, 2H), 7.25 (br d, J=7.63 Hz, 1H), 7.30-7.37 (m, 1H), 7.38-7.45 (m, 4H).

### Example 75F: methyl [{(7S)-3-(benzyloxy)-7-[(tert-butoxycarbonyl)amino]-1-fluoro-7,8-dihydronaphthalen-2-yl}(trifluoroacetyl)amino]acetate

To a solution of the product of Example 75E (7 g, 10.85 mmol, purity 90%) in dichloromethane (300 mL) was added dichloro[1,3-bis(2,4,6-trimethylphenyl)-2-imidazolidinylidene](benzylidene)(tricyclohexylphosphine)ruthenium(II) (1.843 g, 2.170 mmol) at 25° C. The mixture was stirred in the dark at 40° C. for 6 hours. One additional reaction on 1 g scale was run as described above. The reaction mixtures were concentrated under reduced pressure. The crude residue was purified by column chromatography on silica gel eluting with ethyl acetate in petroleum ether from 15% to 25% to give the title compound (7 g, 11.40 mmol, yield 92%, purity 90%). ¹H NMR (400 MHZ, DMSO-d₆) δ ppm 7.49-7.31 (m, 4H), 7.29-7.14 (m, 1H), 7.00 (s, 1H), 6.53 (br d, J=8.3 Hz, 1H), 6.15-5.93 (m, 1H), 5.30-5.06 (m, 2H), 4.50 (br dd, J=5.3, 16.9 Hz, 2H), 4.40-4.15 (m, 3H), 3.60 (br d, J=2.5 Hz, 4H), 3.06-2.83 (m, 1H), 2.73-2.60 (m, 1H), 1.59-1.29 (m, 8H).

### Example 75G: methyl ({(7S)-3-(benzyloxy)-7-[(tert-butoxycarbonyl)amino]-1-fluoro-7,8-dihydronaphthalen-2-yl}amino)acetate

To a solution of the product of Example 75F (3 g, 4.89 mmol, purity 90%) in methanol (35 mL) was added sodium methoxide (1.056 g, 5.86 mmol) at 25° C. The mixture was stirred at 60° C. for 3 hours. The mixture was poured into saturated aqueous NH₄Cl (20 mL), and the resultant mixture was extracted with ethyl acetate (3×40 mL). The combined organic layers were washed with brine (80 mL), dried over Na₂SO₄, and concentrated under reduced pressure. The residue was purified by column chromatography on silica gel eluted with ethyl acetate in petroleum ether from 12% to 15% to give the title compound (1.5 g, 2.96 mmol, yield 60.5%, purity 90%). ¹H NMR (400 MHZ, CDCl₃) δ ppm 7.50-7.32 (m, 5H), 6.48 (s, 1H), 6.38 (d, J=9.6 Hz, 1H), 5.87 (br dd, J=4.7, 9.2 Hz, 1H), 5.09 (s, 2H), 4.62 (br d, J=7.9 Hz, 2H), 4.45 (br s, 1H), 4.12 (d, J=1.6 Hz, 2H), 3.75 (s, 3H), 2.91 (br t, J=7.0 Hz, 2H), 1.50-1.41 (m, 9H).

### Example 75H: methyl [{(7S)-3-(benzyloxy)-7-[(tert-butoxycarbonyl)amino]-1-fluoro-7,8-dihydronaphthalen-2-yl}({[(prop-2-en-1-yl)oxy]carbonyl}sulfamoyl)amino]acetate

To a solution of chlorosulfonyl isocyanate (1.244 g, 8.79 mmol) in dichloromethane (15 mL) was added allyl alcohol (0.51 g, 8.78 mmol) dropwise at 0° C. The mixture was stirred at 0° C. for 30 minutes. Then a solution of the product of Example 75G (1.3 g, 2.56 mmol, purity 90%) and N,N-diisopropylethylamine (1.656 g, 12.81 mmol) in dichloromethane (10 mL) was added dropwise into the above mixture at 0° C. The resulting mixture was stirred at 0° C. for 30 minutes. The mixture was poured into water (30 mL). The mixture was extracted with dichloromethane (3× 20 mL), dried over Na₂SO₄, and concentrated under reduced pressure to give the title compound (2 g), which was used in the next step directly. MS (ESI⁺) m/z 642 [M+23]⁺.

### Example 75I: tert-butyl [(2S)-6-(benzyloxy)-8-fluoro-7-(1,1,4-trioxo-1λ⁶,2,5-thiadiazolidin-2-yl)-1,2-dihydronaphthalen-2-yl]carbamate

To a solution of the product of Example 75H (1.9 g, 3.07 mmol, crude) in methanol (30 mL) was added K₂CO₃ (1.271 g, 9.20 mmol) followed by tetrakis(triphenylphosphine)palladium(0) (0.177 g, 0.153 mmol) at 20° C. under N₂. The mixture was stirred under N₂ at 20° C. for 12 hours. One additional reaction on 0.1 g scale was run as described above. The reaction mixtures were combined and diluted with water (30 mL), then the resultant mixture was extracted with ethyl acetate (3×30 mL). The combined organic layers were washed with brine (80 mL), dried over Na₂SO₄ and concentrated under reduced pressure. The crude residue was purified by chromatography on silica gel eluting with 80% to 100% of ethyl acetate in petroleum ether followed by 1% to 15% methanol in ethyl acetate to give the title compound (1.3 g, 2.324 mmol, yield 72.0% for two steps, purity 90%). ¹H NMR (400 MHZ, DMSO-d₆) δ ppm 7.50 (d, J=7.0 Hz, 2H), 7.41-7.26 (m, 3H), 7.20 (br d, J=7.4 Hz, 1H), 6.81 (s, 1H), 6.46 (br d, J=9.8 Hz, 1H), 5.94 (dd, J=3.3, 9.7 Hz, 1H), 5.13 (s, 2H), 4.28 (br d, J=5.4 Hz, 1H), 3.97 (s, 2H), 2.96-2.53 (m, 2H), 1.40 (s, 9H).

### Example 75J: tert-butyl [(1aS,2S,7bR)-6-(benzyloxy)-4-fluoro-5-(1,1,4-trioxo-1λ⁶,2,5-thiadiazolidin-2-yl)-1a,2,3,7b-tetrahydronaphtho[1,2-b]oxiren-2-yl]carbamate

To a mixture of the product of Example 751 (1 g, 1.787 mmol, purity 90%) and sodium hydrogen carbonate (0.300 g, 3.57 mmol) in dichloromethane (20 mL) was added 3-chloroperoxy benzoic acid (0.544 g, 2.68 mmol) in portions at 0° C. The mixture was stirred for 3 hours at 0° C. The mixture was used directly without any workup.

### Example 75K: tert-butyl [(2S,3R)-8-fluoro-3,6-dihydroxy-7-(1,1,4-trioxo-1λ⁶,2,5-thiadiazolidin-2-yl)-1,2,3,4-tetrahydronaphthalen-2-yl]carbamate

To a mixture of 10% Pd—C (951 mg, 0.894 mmol) in methanol (15 mL) and tetrahydrofuran (15 mL) was added the product of Example 75J (928 mg, 1.787 mmol) in dichloromethane at 25° C. under argon. The mixture was stirred under H₂ (15 psi) at 25° C. for 12 hours. The mixture was diluted with methanol (20 mL) and filtered. The filtrate was concentrated with a stream of N₂. The crude product was purified by revere phase flash chromatography (Agela Claricep™ Flash AQ C18 Column, 20-35 μm, 100 Å, 330 g, Flow rate 100 mL/minute, eluted with 30% gradient of acetonitrile in water) to give the title compound (300 mg, 0.626 mmol, yield 35.0% for two steps, purity 90%), MS (ESI⁻) m/z 430 [M−H]⁻.

### Example 75L: 5-[(6R,7S)-7-amino-1-fluoro-3,6-dihydroxy-5,6,7,8-tetrahydronaphthalen-2-yl]-1λ⁶,2,5-thiadiazolidine-1,1,3-trione

To a solution of the product of Example 75K (100 mg, 0.209 mmol) in dichloromethane (15 mL) was added trifluoroacetic acid (3 mL, 38.9 mmol) dropwise at 0° C. After addition, the mixture was stirred at 0° C. for 3 hours before it was concentrated under reduced pressure to give the title compound which was used directly without purification. MS (ESI⁺) m/z 332 [M+H]⁺.

### Example 75M: 5-{(6R,7S)-1-fluoro-3,6-dihydroxy-7-[(3-methylbutyl)amino]-5,6,7,8-tetrahydronaphthalen-2-yl}-1λ⁶,2,5-thiadiazolidine-1,1,3-trione

To a solution of the product of Example 75L (crude, 69.2 mg, 0.209 mmol) in dichloromethane (5 mL) and ethanol (5 mL) was added triethylamine (0.117 mL, 0.836 mmol) at 25° C. Then a solution of 3-methylbutanal (54.0 mg, 0.627 mmol) in dichloromethane (5 mL) was added dropwise at 25° C., and the mixture was stirred at 25° C. for 2 hours. NaBH₄ (31.6 mg, 0.836 mmol) was then added to the mixture at 0° C. in portions and the resulting mixture was stirred at 25° C. for 20 minutes. The reaction mixture was concentrated under reduced pressure, and the residue was purified by preparative HPLC on a Phenomenex® Gemini®-NX C18, 75×30 mm, 3 μm column eluted with acetonitrile-10 mM NH₄HCO₃ in H₂O with a gradient 5-25% for 12 minutes and 25-100% for 2 minutes) at a flow rate of 25 mL/minute to give the title compound (26.4 mg, 0.064 mmol, yield 30.8% for two steps, purity 97.78%). ¹H NMR (400 MHz, DMSO-d₆) δ ppm 6.46 (s, 1H), 4.28 (br s, 1H), 3.95 (s, 2H), 3.46-3.37 (m, 1H), 3.11-2.93 (m, 3H), 2.86-2.76 (m, 1H), 2.64-2.60 (m, 1H), 1.63 (qd, J=6.6, 13.1 Hz, 1H), 1.57-1.45 (m, 2H), 0.89 (d, J=6.4 Hz, 6H).

### Example 76: 5-(7-{[(3-cyclopropylpropyl)amino]methyl}-1-fluoro-3-hydroxy-5,6,7,8-tetrahydronaphthalen-2-yl)-1λ⁶,2,5-thiadiazolidine-1,1,3-trione (Compound 394)

### Example 76A: tert-butyl {[8-fluoro-6-hydroxy-7-(1,1,4-trioxo-1λ⁶,2,5-thiadiazolidin-2-yl)-1,2,3,4-tetrahydronaphthalen-2-yl]methyl}carbamate

The product of Example 151 (60 mg, 0.115 mmol), ammonium formate (61.1 mg, 0.969 mmol), and 10% Pd/C (12 mg, 0.011 mmol) in ethanol (3 mL) was heated to 65° C. for 1 hour and 25 minutes. The reaction mixture was cooled to ambient temperature and filtered over diatomaceous earth, and the filter cake was rinsed with methanol. The filtrate was concentrated under reduced pressure and purified by reverse phase column chromatography (30 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=25 mL/minute) to give the title compound (45.1 mg, 0.101 mmol, 87% yield). MS (ESI⁻) m/z 428 [M−H]⁻.

### Example 76B: 5-[7-(aminomethyl)-1-fluoro-3-hydroxy-5,6,7,8-tetrahydronaphthalen-2-yl]-1λ⁶,2,5-thiadiazolidine-1,1,3-trione

2,2,2-Trifluoroacetic acid (200 μL, 2.60 mmol) was added to a solution of the product of Example 76A (45.1 mg, 0.105 mmol) in dichloromethane (2 mL) and the mixture was stirred at ambient temperature for 2 hours. The reaction mixture was concentrated under reduced pressure, and the residue was azeotroped with toluene (3×2 mL). The residue was triturated with acetonitrile to give the title compound as a trifluoracetic acid salt (16.8 mg, 0.038 mmol, 36.1% yield). ¹H NMR (600 MHZ, DMSO-d₆) δ ppm 9.05 (s, 1H), 7.78 (s, 3H), 6.44 (d, J=1.2 Hz, 1H), 3.94 (d, J=2.3 Hz, 2H), 2.90-2.80 (m, 3H), 2.77-2.70 (m, 1H), 2.67-2.60 (m, 1H), 2.20 (dd, J=16.6, 10.6 Hz, 1H), 1.93-1.86 (m, 2H), 1.39-1.29 (m, 1H); MS (ESI⁺) m/z 330 [M+H]⁺.

### Example 76C: 5-(7-{[(3-cyclopropylpropyl)amino]methyl}-1-fluoro-3-hydroxy-5,6,7,8-tetrahydronaphthalen-2-yl)-1λ⁶,2,5-thiadiazolidine-1,1,3-trione

Triethylamine (20 μL, 0.142 mmol) was added to the product of Example 76B (15.7 mg, 0.035 mmol) in ethanol (2 mL). The reaction mixture stirred at ambient temperature for 5 minutes, after which 3-cyclopropylpropanal (19 mg, 0.194 mmol) dissolved in dichloromethane (1 mL) was added. The resultant mixture was stirred further for 3 hours at ambient temperature. Sodium tetrahydroborate (13.5 mg, 0.357 mmol) was then added and the mixture was stirred for 1 hour. The reaction was quenched with 1 M HCl (0.5 mL) and the mixture was concentrated under reduced pressure with diatomaceous earth for dry loading. The residue was purified by reverse phase column chromatography (30 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=25 mL/minute) to afford the title compound (4.2 mg, 10.21 μmol, 28.8% yield). ¹H NMR (600 MHZ, DMSO-d₆) δ ppm 9.01 (s, 1H), 8.03 (s, 2H), 6.44 (s, 1H), 3.97-3.89 (m, 2H), 2.95-2.89 (m, 4H), 2.85 (dd, J=16.3, 5.1 Hz, 1H), 2.73 (dt, J=17.2, 4.5 Hz, 1H), 2.69-2.61 (m, 1H), 2.21 (dd, J=16.5, 10.3 Hz, 1H), 1.99-1.96 (m, 1H), 1.92-1.87 (m, 1H), 1.73-1.65 (m, 2H), 1.36 (dtd, J=12.7, 11.0, 5.3 Hz, 1H), 1.23 (q, J=7.2 Hz, 2H), 0.74-0.64 (m, 1H), 0.45-0.37 (m, 2H), 0.05-0.01 (m, 2H); MS (ESI⁺) m/z 412 [M+H]⁺.

### Example 77: tert-butyl [(2R,4R)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate (Compound 395)

### Example 77A: (2R)-2-[(tert-butoxycarbonyl)amino]-4-{[tert-butyl(dimethyl)silyl]oxy}butanoic acid

To a stirred solution of (2R)-2-[(tert-butoxy carbonyl)amino]-4-hydroxy butanoic acid (10.55 g, 48.1 mmol), 1H-imidazole (6.55 g, 96 mmol) and N,N-dimethylformamide (96 mL) was added tert-butylchlorodimethylsilane (7.25 g, 48.1 mmol) in one portion. After stirring overnight, the reaction mixture was concentrated. The residue was taken up in tert-butyl methyl ether and washed with aqueous 1 M HCl and brine, dried over Na₂SO₄, and concentrated to give the title compound (16.6 g, 49.6 mmol, 103% yield), which was carried on to next step without further purification (assumed 100% yield). MS (APCI⁻) m/z 322 [M−H]⁻.

### Example 77B: tert-butyl [(2R)-4-{[tert-butyl(dimethyl)silyl]oxy}-1-hydroxybutan-2-yl]carbamate

To a stirred solution of the product of Example 77A (12.17 g, 36.5 mmol) in tetrahydrofuran (182 mL) at 0° C. was added 4-methylmorpholine (4.21 mL, 38.3 mmol) and isobutyl carbonochloridate (5.11 mL, 38.3 mmol). After 30 minutes, the reaction mixture was filtered through a plug of diatomaceous earth, washing the filter cake with additional tetrahydrofuran. A solution of sodium borohydride (2.76 g, 73.0 mmol) in water (45 mL) was added to a stirred solution of the combined filtrates. After 1 hour the reaction was quenched by addition of aqueous 1 M HCl and partially concentrated. The mixture was transferred to a separatory funnel and extracted three times with ethyl acetate. The combined organic fractions were washed with brine, dried over Na₂SO₄ and concentrated. The residue was loaded onto a 220 g Teledyne ISCO column and purified using a gradient of 12-100% ethyl acetate in heptanes, yielding the title compound (12.0 g, 37.6 mmol, 103% yield). MS (APCI⁺) m/z 320 [M+H]⁺.

### Example 77C: ter-butyl (4R)-4-(2-{[ter-butyl(dimethyl)silyl]oxy}ethyl)-2-oxo-1,2λ⁴,3-oxathiazolidine-3-carboxylate

A solution of thionyl chloride (3.29 mL, 45.1 mmol) in dichloromethane (16 mL) was added slowly to a solution of imidazole (10.23 g, 150 mmol) and triethylamine (15.70 mL, 113 mmol) in dichloromethane (114 mL) at −40° C. The mixture was stirred for 15 minutes at −40° C., during which time a thick slurry formed. A solution of the product of Example 77B (12.0 g, 37.6 mmol) in dichloromethane (26 mL) was added over a 30 minute period, maintaining the cooling bath at −40° C. The reaction mixture was allowed to warm to ambient temperature and stirred overnight. The reaction mixture was slowly diluted with a saturated aqueous solution of NaHCO₃ and transferred to a separatory funnel. The layers were separated, and the organic layer was washed with brine, dried over Na₂SO₄, and concentrated. To remove residual imidazole, the residue was taken up in tert-butyl methyl ether and washed three times with water, before drying over Na₂SO₄ and concentrating to give the title compound (13.8 g, 37.8 mmol, 101% yield) which was carried on to the next step without further purification (assumed 100% yield). ¹H NMR (600.4 MHZ, CDCl₃) δ ppm 4.86-4.76 (m, 2H), 3.92 (tdd, J=9.4, 7.2, 2.9 Hz, 1H), 3.70 (dt, J=10.7, 4.6 Hz, 1H), 3.60 (ddd, J=10.7, 9.4, 3.6 Hz, 1H), 1.83-1.74 (m, 1H), 1.47 (s, 9H), 0.84 (d, J=1.1 Hz, 9H), 0.00 (d, J=1.7 Hz, 6H); MS (APCI+) m/z 366 [M+H]⁺.

### Example 77D: tert-butyl (4R)-4-(2-{[tert-butyl(dimethyl)silyl]oxy}ethyl)-2,2-dioxo-1,2λ⁶,3-oxathiazolidine-3-carboxylate

To a solution of the product of Example 77C (13.8 g, 37.8 mmol) in acetonitrile (101 mL) and water (25.2 mL) was added ruthenium(III) chloride hydrate (0.078 g, 0.378 mmol) and sodium periodate (8.88 g, 41.5 mmol). The reaction mixture was stirred for 3 minutes at 23° C. before dilution with ethyl acetate (200 mL). The diluted mixture was filtered through a polyethylene frit packed with diatomaceous earth and the filter cake was washed with ethyl acetate three times. The combined filtrates were transferred to a separatory funnel and washed with saturated aqueous sodium thiosulfate solution (150 mL) and brine, dried over MgSO₄, and concentrated. The residue was loaded onto a 120 g Teledyne ISCO silica gel column and purified using a gradient of 2-20% ethyl acetate in heptanes to yield the title compound (10.4 g, 27.2 mmol, 72%). ¹H NMR (500.2 MHZ, CDCl₃) δ ppm 4.69-4.63 (m, 2H), 4.39 (ddt, J=9.0, 5.5, 3.3 Hz, 1H), 3.81 (ddd, J=10.8, 5.5, 4.4 Hz, 1H), 3.74 (ddd, J=10.8, 8.5, 3.8 Hz, 1H), 2.19-2.11 (m, 1H), 2.09-1.98 (m, 1H), 1.56 (s, 9H), 0.89 (s, 9H), 0.06 (s, 3H), 0.06 (s, 3H); MS (APCI⁺) m/z 399 [M+NH₄]⁺.

### Example 77E: 1-(benzyloxy)-5-bromo-3-fluoro-2-nitrobenzene

To a suspension of 5-bromo-1,3-difluoro-2-nitrobenzene (40 g, 168 mmol) and benzyl alcohol (18.4 mL, 176 mmol) in tetrahydrofuran (800 mL) at −60° C. was added a solution of potassium tert-butoxide (176 mL, 176 mmol, 1 M in tetrahydrofuran) slowly along the side of the flask so that the internal temperature remained below −50° C. After complete addition, the mixture was stirred for 5 minutes, then was quenched with saturated aqueous ammonium chloride (40 mL), diluted with water (200 mL) and ethyl acetate (200 mL) and warmed to ambient temperature. The aqueous layer was extracted with ethyl acetate (200 mL). The combined organic fractions were washed with brine (160 mL), dried over anhydrous sodium sulfate, filtered and concentrated in vacuo. Heptanes (500 mL) were added to the crude solid, the mixture was heated to an internal temperature of 65° C., then slowly cooled to ambient temperature, and the solids were collected by filtration. The solids were washed with the cold mother liquor and additional heptane (120 mL) and then were dried in a vacuum oven at 60° C. to constant weight to give 39.95 g of the title compound. The mother liquor was concentrated and then solids were precipitated from heptanes (100 mL) to give an additional 7.56 g of the title compound. Total recovery of the title compound was 47.5 g (146 mmol, 87% yield). ¹H NMR (400 MHZ, DMSO-d₆) δ ppm 7.63 (t, J=1.7 Hz, 1H), 7.57 (dd, J=9.3, 1.7 Hz, 1H), 7.46-7.32 (m, 5H), 5.36 (s, 2H).

### Example 77F: 2-(benzyloxy)-4-bromo-6-fluoroaniline

To a suspension of the product from Example 77E (5.68 g, 17.4 mmol) and zinc dust (5.70 g, 87 mmol) in a mixture of tetrahydrofuran (56.8 mL) and methanol (56.8 mL) was added saturated aqueous ammonium chloride (28.4 mL) slowly via addition funnel so that the internal temperature remained below 30° C. After stirring vigorously for 1 hour, the mixture was filtered through diatomaceous earth (5 g), and the solids were washed with ethyl acetate (56.8 mL). The filtrate was washed with brine (56.8 mL), and then the aqueous layer was extracted with ethyl acetate (28.4 mL). The combined organic layers were washed with water (28.4 mL) and then brine (22.7 mL), dried over anhydrous sodium sulfate, filtered and concentrated in vacuo to give the title compound (5.2 g, 17.5 mmol, 100% yield) which was used for the next step without purification. ¹H NMR (400 MHZ, DMSO-d₆) δ ppm 7.52-7.45 (m, 2H), 7.43-7.36 (m, 2H), 7.36-7.30 (m, 1H), 6.99-6.93 (m, 2 h), 5.16 (s, 2H), 4.83 (s, 2H); MS (ESI⁺) m/z 296 [M+H]⁺.

### Example 77G: N-[2-(benzyloxy)-4-bromo-6-fluorophenyl]-2,2,2-trifluoroacetamide

To a solution of the product from Example 77F (5.6 g, 18.96 mmol) and pyridine (2.30) mL, 28.4 mmol) in acetonitrile (56 mL) at an internal temperature below 16° C. was added trifluoroacetic anhydride (3.48 mL, 24.6 mmol), slowly. After 5 minutes, the reaction mixture was diluted with dichloromethane (56 mL) and water (56 mL). The aqueous layer was extracted with dichloromethane (28 mL), and the combined organic layers were washed with brine (28 mL), dried over anhydrous sodium sulfate, filtered and concentrated in vacuo to give the title compound (7.41 g, 18.9 mmol, 100% yield) which was used for the next step without purification. ¹H NMR (400 MHZ, DMSO-d₆) δ ppm 11.04 (s, 1H), 7.45-7.29 (m, 8H), 5.24 (s, 2H); MS (ESI⁻) m/z 390 [M−H]⁻.

### Example 77H; tert-butyl [(2S)-1-[4-(benzyloxy)-6-bromo-2-fluoro-3-(2,2,2-trifluoroacetamido)phenyl]-4-{[tert-butyl(dimethyl)silyl]oxy}butan-2-yl]carbamate

A 250 mL round bottom flask was charged with tetrahydrofuran (67.5 mL), cooled to −78° C., and charged with diisopropylamine (8.46 mL, 59.4 mmol), followed by dropwise addition of n-butyllithium (2.50 M, 22.68 mL, 56.7 mmol). After stirring for 15 minutes, a solution of the product from Example 77G in tetrahydrofuran (33.7 mL) was added dropwise over 20 minutes, and the resultant mixture was stirred for 30 minutes. Subsequently, a solution of the product from Example 77D (10.3 g, 27.0 mmol) in tetrahydrofuran (33.7 mL) was added dropwise, and the mixture was stirred for an additional 30 minutes at −78° C. The reaction was quenched at −78° C. by addition of aqueous 1 M HCl (67.5 mL, 135 mmol), and the mixture was allowed to warm to ambient temperature. The mixture was diluted with ethyl acetate and transferred to a separatory funnel, where it was washed with water and brine. The organic fraction was dried over Na₂SO₄ and concentrated. The residue was loaded onto a 220 g Teledyne ISCO silica gel column and purified with a gradient of 2-20% ethyl acetate in heptanes to give the title compound (8.4 g, 12.1 mmol, 45% yield). ¹H NMR (499.6 MHZ, CDCl₃) δ ppm 7.58 (s, 1H), 7.42-7.33 (m, 5H), 7.05 (s, 1H), 5.21-5.01 (m, 2H), 4.10-4.04 (m, 1H), 3.84 (s, 1H), 3.76-3.70 (m, 1H), 3.09-2.98 (m, 1H), 2.95-2.85 (m, 1H), 1.90-1.80 (m, 1H), 1.66 (d, J=5.4 Hz, 1H), 1.31 (s, 9H), 0.91 (s, 9H), 0.07 (d, J=5.3 Hz, 6H); MS (APCI+) m/z 693 [M+H]⁺.

### Example 77I: methyl [{6-(benzyloxy)-4-bromo-3-[(2S)-2-[(tert-butoxycarbonyl)amino]-4-{[tert-butyl(dimethyl)silyl]oxy}butyl]-2-fluorophenyl}(trifluoroacetyl)amino]acetate

A 100 mL round bottom flask was charged with the product from Example 77H (8.4 g, 12.11 mmol), acetone (60.6 mL), potassium carbonate (5.02 g, 36.3 mmol), potassium iodide (1.005 g, 6.06 mmol), and methyl bromoacetate (1.228 mL, 13.32 mmol), and the mixture was vigorously stirred under N₂ at ambient temperature. After 4 hours, the reaction was diluted with ethyl acetate and transferred to a separatory funnel. The solution was washed with water, and the aqueous layer was back extracted with ethyl acetate. The combined organic layers were washed with brine and concentrated. The residue was loaded onto a 220 g Teledyne ISCO silica gel column and purified with a gradient of 10-80% ethyl acetate in heptanes to give the title compound (8.5 g, 11.1 mmol, 92% yield). MS (APCI⁺) m/z 766 [M+H]⁺.

### Example 77J: methyl {[6-(benzyloxy)-4-bromo-3-{(2S)-2-[(tert-butoxycarbonyl)amino]-4-hydroxybutyl}-2-fluorophenyl](trifluoroacetyl)amino}acetate

A 50 mL flask was charged with the product from Example 771 (0.800 g, 1.045 mmol), acetic acid (7.8 mL), tetrahydrofuran (2.6 mL), and water (2.6 mL) and the mixture was rapidly stirred at 40° C. After two hours, the mixture was cooled to ambient temperature and carefully diluted with ethyl acetate and saturated aqueous sodium bicarbonate. The mixture was transferred to a separatory funnel and the organic layer was separated. The aqueous layer was extracted with ethyl acetate three times, and the combined organic layers were dried over Na₂SO₄ and concentrated. The residue was loaded onto a Teledyne ISCO silica gel column and purified with a gradient of 10-80% ethyl acetate in heptanes to give the title compound (520 mg, 0.798 mmol, 76% yield). MS (APCI⁺) m/z 651 [M+H]⁺.

### Example 77K: methyl {[6-(benzyloxy)-4-bromo-3-{(2S)-2-[(tert-butoxycarbonyl)amino]-4-oxobutyl}-2-fluorophenyl](trifluoroacetyl)amino}acetate

A 25 mL round bottom flask was charged with the product of Example 77J (1.29 g, 1.980 mmol) and dichloromethane (19.88 mL). After cooling to 0° C., (1,1,1-tris (acetyloxy)-1,1-dihydro-1,2-benzodioxol-3-(1H)-one) (DMP, 1.680 g, 3.96 mmol) in dichloromethane (3.98 mL) was added dropwise. After 15 minutes the mixture was diluted with water and ethyl acetate and filtered through a polyethylene frit packed with diatomaceous earth. The filtrate was transferred to a separatory funnel, and the layers were separated. The organic layer was washed with brine, dried over Na₂SO₄, and concentrated. The residue was loaded onto a Teledyne ISCO silica gel column and was purified with a 20-60% gradient of ethyl acetate in heptanes to yield the title compound (1.14 g, 1.755 mmol, 89% yield). MS (APCI⁺) m/z 666 [M+NH₄]⁺.

### Example 77L: methyl {[6-(benzyloxy)-4-bromo-3-{(2R)-2-[(tert-butoxycarbonyl)amino]pent-4-en-1-yl}-2-fluorophenyl](trifluoroacetyl)amino}acetate

A heat dried 50 mL round bottom flask was charged with methyltriphenylphosphonium bromide (0.658 g, 1.843 mmol) and toluene (8.78 mL) and cooled to 0° C. in a dry-ice acetone bath under N₂. A solution of sodium bis(trimethylsilyl)amide (2.93 mL, 1.755 mmol) was added dropwise and the resulting solution was stirred for 25 minutes at 0° C. before cooling to −78° C. A solution of the product of Example 77K (845 mg, 1.305 mmol, 74.3% yield) in toluene (1.756 mL) was added in one portion and stirred for 30 minutes before allowing the reaction mixture to warm to ambient temperature. The reaction was quenched with a saturated aqueous solution of NH₄Cl, the mixture was transferred to a separatory funnel and extracted three times with ethyl acetate. The combined organic layers were washed with brine, dried over MgSO₄, and concentrated onto 10 g of SiO₂. The residue was loaded onto an 80 g Teledyne ISCO silica gel column and purified with a gradient of 10-50% ethyl acetate in heptanes to yield the title compound (845 mg, 1.305 mmol, 74.3% yield). MS (APCI⁺) m/z 648 [M+H]⁺.

### Example 77M: methyl [{(7S)-3-(benzyloxy)-7-[(tert-butoxycarbonyl)amino]-1-fluoro-5-methyl-7,8-dihydronaphthalen-2-yl}(trifluoroacetyl)amino]acetate

A 50 mL round bottom flask was charged with the product of Example 77L (0.405 g, 0.626 mmol), 1,4-dioxane (12.51 mL), palladium(II)acetate (0.014 g, 0.063 mmol), triphenylphosphine (0.033 g, 0.125 mmol), and potassium carbonate (0.519 g, 3.75 mmol). The reaction mixture was sparged for 30 minutes with N₂ and then heated to 90° C. on a preheated reaction block. After 3 hours, the reaction mixture was cooled to ambient temperature, filtered through a polyethylene frit packed with diatomaceous earth, and concentrated onto 5 g of SiO₂. The residue was dry loaded onto a 24 g Teledyne ISCO silica gel column and purified with a 5-20% gradient of ethyl acetate in heptanes to yield the title compound (220 mg, 0.388 mmol, 62.1% yield). MS (APCI⁺) m/z 584 [M+NH₄]⁺.

### Example 77N: methyl ({(7S)-3-(benzyloxy)-7-[(tert-butoxycarbonyl)amino]-1-fluoro-5-methyl-7,8-dihydronaphthalen-2-yl}amino)acetate

A 20 mL screw top vial was charged with the product of Example 77M (220 mg, 0.388 mmol) and sodium methoxide (0.5 M in methanol 2330 μL, 1.165 mmol). The vial was heated to 60° C. on a preheated reaction block. After 1 hour, the reaction was quenched by addition of saturated aqueous NH₄Cl/water (1:1) and transferred to a separatory funnel with ethyl acetate. The layers were separated, and the aqueous layer was extracted three times with ethyl acetate. The combined organic layers were washed with brine, dried over Na₂SO₄, and concentrated to afford the title compound which was used without further purification in the next step. MS (APCI⁺) m/z 471 [M+H]⁺.

### Example 77O: methyl [{(7S)-3-(benzyloxy)-7-[(tert-butoxycarbonyl)amino]-1-fluoro-5-methyl-7,8-dihydronaphthalen-2-yl}({[(prop-2-en-1-yl)oxy]carbonyl}sulfamoyl)amino]acetate

A heat-dried 10 mL round bottom flask was charged with dichloromethane (1296 μL) and chlorosulfonyl isocyanate (50.7 μL, 0.583 mmol), and the mixture was cooled to −20° C. in a dry ice/acetone bath. Allyl alcohol (39.7 μL, 0.583 mmol) was added dropwise over a 5 minute period. After 30 minutes, a solution of the product of Example 77N (183 mg, 0.389 mmol) and triethylamine (136 μL, 0.9725 mmol) in dichloromethane (648 μL) was added via syringe. After 5 minutes, the reaction mixture was diluted with ethyl acetate and water, and transferred to a separatory funnel. The layers were separated, and the aqueous layer was back extracted with ethyl acetate three times. The combined organic solutions were dried over Na₂SO₄ and concentrated onto 2 g of SiO₂. The residue was dry loaded onto a 24 g Teledyne ISCO silica gel column and purified with a gradient of 25-95% ethyl acetate in heptanes to yield the title compound (187 mg, 0.295 mmol, 76% yield). MS (ESI⁺) m/z 651 [M+NH₄]⁺.

### Example 77P: tert-butyl [(2S)-6-(benzyloxy)-8-fluoro-4-methyl-7-(1,1,4-trioxo-1λ⁶,2,5-thiadiazolidin-2-yl)-1,2-dihydronaphthalen-2-yl]carbamate

A 1 dram vial was charged with the product of Example 770 (187 mg, 0.295 mmol), tetrakis(triphenylphosphine)palladium(0)) (5.9 mg, 5.11 μmol), and a solution of sodium methoxide (0.5 M in methanol 1532 μL, 0.766 mmol). The vial was sparged for 5 minutes before being placed in a preheated reaction block at 60° C. After 30 minutes, the reaction mixture was cooled to ambient temperature and the reaction was quenched by addition of 4 M HCl in 1,4-dioxane (49 μL, 0.197 mmol). The mixture was transferred to a separatory funnel and diluted with brine and ethyl acetate. The layers were separated, and the aqueous layer was back extracted with ethyl acetate three times. The combined organic layers were dried over Na₂SO₄ and filtered through a polyethylene frit packed with diatomaceous earth, washing the frit three times with ethyl acetate. The combined filtrate and washes were concentrated to give the title compound (143 mg, 0.276 mmol, 108% yield), which was used in the next step without further purification (assumed 100% yield). MS (APCI⁺) m/z 535 [M+NH₄]⁺.

### Example 77Q: tert-butyl [(2R)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate

A 1 dram vial was charged with the product of Example 77P (153 mg, 0.295 mmol), ammonium formate (130 mg, 2.065 mmol), Pd/C (10 weight %, 94 mg, 0.0885 mmol), and ethanol (1475 μL). The vial was flushed with N₂, sealed, and heated to 60° C. After 30 minutes the reaction mixture was cooled to ambient temperature and passed through a polyethylene frit packed with diatomaceous earth, washing the frit with ethanol twice. The combined washes and filtrate were concentrated, and the residue was passed through a small pad of SiO₂ in ethyl acetate/ethanol (4:1) and concentrated. The obtained residue was loaded onto a 12 g Teledyne ISCO silica gel column in 1:1 ethyl acetate/ethanol and purified with a gradient of methanol in ethyl acetate (5-100%) to yield a mixture of cis and trans diastereomers in a 2:1 ratio (110 mg, 0.256 mmol 87% yield). MS (APCI⁺) 447 [M+NH₄]⁺.

### Example 77R: tert-butyl [(2R,4R)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate

The product of Example 77Q (110 mg, 0.256 mmol) was separated by preparative chiral SFC. Preparative SFC was performed on a Waters SFC80Q SFC running under ChromScope™ software control. The preparative SFC system was equipped with a CO₂ pump, modifier pump with 4-port solvent selection valve, automated back pressure regulator (ABPR). UV detector, and 6-position fraction collector. The mobile phase was comprised of supercritical CO₂ supplied by a Dewar of bone-dry non-certified CO₂ pressurized to 350 psi with a modifier of methanol (0.1% diethylamine) at a flow rate of 80 g/minute. The column was held at ambient temperature and the backpressure regulator was set to maintain 100 bar. The sample was dissolved in methanol at a concentration of 18.5 mg/mL. The sample was loaded into the modifier stream in 0.1 mL (1.85 mg) injections. The mobile phase was held isocratically at 30% modifier. Fraction collection was time triggered. The instrument was fitted with a CHIRALPAK® IC column with dimensions 31 mm i.d.×250 mm length with 5 μm particles. The third eluting peak at 18.5 minutes was assigned as the title compound (10 mg, 9% recovery). Retention times were 9.8 minutes, 13.2 minutes (mix), and 18.5 minutes for collected peaks. ¹H NMR (DMSO-d₆) δ ppm 8.48 (bs, 1H), 6.90 (d, J=7.5 Hz, 1H), 6.50 (s, 1H), 3.95-3.90 (m, 2H), 3.79-3.70 (m, 1H), 2.98-2.94 (M, 1H), 2.92 (q, J=7.23, 2H), 2.83 (dd, J=16.2, 5.6 Hz, 1H), 1.40 (s, 9H), 1.21 (d, J=7.2 Hz, 3H); MS (APCI⁺) m/z 447 [M+NH₄]⁺.

### Example 78: 5-{7-[(butylamino)methyl]-1-fluoro-3-hydroxy-5,6,7,8-tetrahydronaphthalen-2-yl}-1λ⁶,2,5-thiadiazolidine-1,1,3-trione (Compound 396)

### Example 78A: 5-{3-(benzyloxy)-7-[(butylamino)methyl]-1-fluoro-5,6,7,8-tetrahydronaphthalen-2-yl}-1λ⁶,2,5-thiadiazolidine-1,1,3-trione

Triethylamine (0.05 mL, 0.359 mmol) was added to the product of Example 15J (41.1 mg, 0.077 mmol) in dichloromethane (1 mL) and ethanol (2 mL). The reaction mixture stirred at ambient temperature for 10 minutes. Then butyraldehyde (0.035 mL, 0.385 mmol) was added and the mixture was stirred further for 2 hours. Sodium tetrahydroborate (23.30 mg, 0.616 mmol) was then added and the mixture was stirred for 1 hour. The reaction mixture was quenched with 1 M HCl (0.3 mL) and concentrated under reduced pressure. The mixture was filtered through a glass microfiber frit, rinsed with a minimal amount of methanol/N,N-dimethylformamide. The resulting filtrate was purified by reverse-phase HPLC [Waters XBridge™ RP 18 column, 5 μm, 30 mm×100 mm, flow rate 40 mL/minute, 5-100% gradient of acetonitrile in buffer 0.025 M aqueous ammonium bicarbonate, adjusted to pH 10 with ammonium hydroxide)] to afford the title compound (26.2 mg, 0.055 mmol, 71.5% yield). MS (ESI⁺) m/z 476 [M+H]⁺.

### Example 78B: 5-{7-[(butylamino)methyl]-1-fluoro-3-hydroxy-5,6,7,8-tetrahydronaphthalen-2-yl}-1λ⁶,2,5-thiadiazolidine-1,1,3-trione

Trichloroborane (1.0 M in dichloromethane) (0.440 mL, 0.440 mmol) was added to a vial containing a suspension of the product of Example 78A (26.2 mg, 0.055 mmol) and 1,2,3,4,5-pentamethylbenzene (25 mg, 0.169 mmol) in dichloromethane (2 mL) cooled to −78° C. The mixture was stirred at −78° C. for 10 minutes, and then at 0° C. for 40 minutes. The reaction mixture was recooled to −78° C. and quenched with the successive addition of ethyl acetate (2 mL) and ethanol (2 mL). The mixture was then allowed to warm to ambient temperature and stirred further for 15 minutes. The mixture was concentrated under reduced pressure, and the residue was filtered through a glass microfiber frit that was then rinsed with a minimal amount of methanol/N,N-dimethylformamide. The resulting filtrate was purified by reverse-phase HPLC [Waters XBridge™ RP 18 column, 5 μm, 30 mm×100 mm, flow rate 40 mL/minute, 3-100% gradient of acetonitrile in buffer 0.025 M aqueous ammonium bicarbonate, adjusted to pH 10 with ammonium hydroxide)] to give the title compound (5.4 mg, 0.014 mmol, 25.4% yield). ¹H NMR (400 MHZ, DMSO-d₆) δ ppm 6.44 (s, 1H), 3.93 (d, J=1.7 Hz, 2H), 2.94-2.79 (m, 5H), 2.74-2.63 (m, 2H), 2.27-2.16 (m, 1H), 1.62-1.51 (m, 2H), 1.56 (p, J=7.7 Hz, 2H), 1.39-1.28 (m, 3H), 0.90 (t, J=7.4 Hz, 3H); MS (ESI⁺) m/z 386 [M+H]⁺.

### Example 79: 5-[(5R,7R)-7-amino-1-fluoro-3-hydroxy-5-methyl-5,6,7,8-tetrahydronaphthalen-2-yl]-1λ⁶,2,5-thiadiazolidine-1,1,3-trione (Compound 397)

A one dram vial was charged with the product of Example 77 (10.3 mg, 0.024 mmol) and acetonitrile (320 μL). Subsequently, a 4 M solution of HCl (6.00 μL, 0.024 mmol) in 1,4-dioxane was added, and the reaction mixture was allowed to stir at ambient temperature overnight, resulting in a heterogenous solution. The reaction mixture was concentrated under a stream of N₂. The crude residue was loaded onto a 12 g Biotage® Sfär C18 column and purified with a gradient of 15-100% acetonitrile in 0.025 M NH₄HCO; in water (acidified to pH 7 by addition of dry ice) to yield the title compound (7.3 mg, 0.022 mmol, 92% yield). ¹H NMR (DMSO-d₆) δ ppm 7.84 (bs, 3H), 6.55 (s, 1H), 3.58-3.50 (m, 1H), 3.06-2.99 (m, 2H), 2.44 (dd, J=16.1, 9.7 Hz, 1H), 1.89-1.87 (m, 2H), 1.23 (d, J=7.20 Hz, 3H); MS (APCI⁺) m/z 330 [M+H]⁺.

### Example 80: 5-[(5S,7R)-7-amino-1-fluoro-3-hydroxy-5-methyl-5,6,7,8-tetrahydronaphthalen-2-yl]-1λ⁶,2,5-thiadiazolidine-1,1,3-trione (Compound 398)

### Example 80A: tert-butyl [(2R,4S)-8-fluoro-6-hydroxy-4-methyl-7-(1,1,4-trioxo-1λ⁶,2,5-thiadiazolidin-2-yl)-1,2,3,4-tetrahydronaphthalen-2-yl]carbamate

The product of Example 77Q (110 mg, 0.256 mmol) was separated by preparative chiral SFC. Preparative SFC was performed on a Waters SFC80Q SFC running under ChromScope™ software control. The preparative SFC system was equipped with a CO₂ pump, modifier pump with 4-port solvent selection valve, automated back pressure regulator (ABPR). UV detector, and 6-position fraction collector. The mobile phase was comprised of supercritical CO₂ supplied by a Dewar of bone-dry non-certified CO₂ pressurized to 350 psi with a modifier of methanol (0.1% diethylamine) at a flow rate of 80 g/minute. The column was held at ambient temperature and the backpressure regulator was set to maintain 100 bar. The sample was dissolved in methanol at a concentration of 18.5 mg/mL. The sample was loaded into the modifier stream in 0.1 mL (1.85 mg) injections. The mobile phase was held isocratically at 30% modifier. Fraction collection was time triggered. The instrument was fitted with a CHIRALPAK® IC column with dimensions 31 mm i.d.×250 mm length with 5 μm particles. The second eluting peak at 13.2 minutes was assigned as the title compound (40 mg, 36% recovery). ¹H NMR (DMSO-d₆) δ ppm 8.40 (bs, 1H), 6.98 (d, J=7.7 Hz, 1H), 6.63 (s, 1H), 3.98-3.91 (m, 2H), 3.60-3.51 (m, 1H), 2.92 (q, J=7.2 Hz, 2H), 2.89-2.81 (m, 2H), 2.29 (dd, J=16.1, 11.3 Hz), 1.42 (s, 9H), 1.24 (d, J=6.9 Hz, 3H); MS (APCI+) m/z 447 [M+NH₄]⁺.

### Example 80B: 5-[(5S,7R)-7-amino-1-fluoro-3-hydroxy-5-methyl-5,6,7,8-tetrahydronaphthalen-2-yl]-1λ⁶,2,5-thiadiazolidine-1,1,3-trione

A 1 dram vial was charged with the product of Example 80A (38 mg, 0.88 mmol) and acetonitrile (0.59 mL). A 4 M solution of HCl in dioxane (0.022 mL, 0.88 mmol) was added, and the reaction mixture was allowed to stir at ambient temperature overnight. Subsequently, the reaction mixture was concentrated in vacuo, and the crude residue was loaded onto a 12 g Biotage® Sfär C18 column and purified with a gradient of 15-100% acetonitrile in 0.025 M NH₄HCO₃ in water (acidified to pH 7 by addition of dry ice) to yield the title compound (9 mg, 0.027 mmol, 31% yield). ¹H NMR (DMSO-d₆) δ ppm 6.88 (s, 1H) 3.96 (s, 2H), 3.05 (dd, J=15.7, 5.3 Hz, 2H), 2.94-2.87 (m, 1H), 2.50-2.47 (m, 1H), 2.17-2.15) 1.44 (q, 1H), 1.28 (d, 3H); MS (APCI+) m/z 330 [M+H]⁺.

### Example 81: 5-(7-{[(cyclopropylmethyl)amino]methyl}-1-fluoro-3-hydroxy-5,6,7,8-tetrahydronaphthalen-2-yl)-1λ⁶,2,5-thiadiazolidine-1,1,3-trione (Compound 399)

Triethylamine (20 μL, 0.144 mmol) was added to the product of Example 76B (16 mg, 0.036 mmol) in ethanol (1 mL) and dichloromethane (0.5 mL). The reaction mixture stirred at ambient temperature for 15 minutes. Then cyclopropanecarbaldehyde (16 μL, 0.217 mmol) was added, and the mixture was stirred for an additional 2 hours. Sodium tetrahydroborate (13.65 mg, 0.361 mmol) was then added, and the resultant mixture was stirred for 50 minutes. More cyclopropanecarbaldehyde (16 μL, 0.217 mmol) was added with continued stirring for 1 hour and 30 minutes. The reaction was quenched with 1 M HCl (0.2 mL), and the mixture was concentrated under reduced pressure. The residue was purified by reverse phase column chromatography (30 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=25 mL/minute) to give the title compound (5.5 mg, 0.014 mmol, 39.7% yield). ¹H NMR (500 MHZ, DMSO-d₆) δ ppm 9.03 (s, 1H), 8.16 (s, 2H), 6.44 (s, 1H), 3.98-3.89 (m, 2H), 2.94 (d, J=6.8 Hz, 2H), 2.89-2.79 (m, 3H), 2.78-2.62 (m, 2H), 2.22 (dd, J=16.5, 10.4 Hz, 1H), 2.01-1.98 (m, 1H), 1.93-1.87 (m, 1H), 1.43-1.31 (m, 1H), 1.09-1.00 (m, 1H), 0.62-0.54 (m, 2H), 0.34 (q, J=5.2 Hz, 2H); MS (ESI⁺) m/z 384 [M+H]⁺.

### Example 82: 5-(7-{[(cyclobutylmethyl)amino]methyl}-1-fluoro-3-hydroxy-5,6,7,8-tetrahydronaphthalen-2-yl)-1λ⁶,2,5-thiadiazolidine-1,1,3-trione (Compound 400)

Triethylamine (0.022 mL, 0.158 mmol) was added to the product of Example 76B (17.5 mg, 0.039 mmol) in ethanol (1 mL) and dichloromethane (0.5 mL). The reaction mixture was stirred at ambient temperature for 15 minutes. Then cyclobutanecarbaldehyde (0.021 mL, 0.237 mmol) was added, and the mixture was stirred for an additional 2 hours. Sodium tetrahydroborate (15 mg, 0.396 mmol) was then added and the resultant mixture was stirred for 30 minutes. The reaction mixture was quenched with 1 M HCl (0.2 mL) and concentrated under reduced pressure. The residue was purified by reverse phase column chromatography (30 g Biotage® Sfär C18 Duo 100 Å 30 μm column. 10 to 100% methanol in water [buffered with 0.025 M aqueous ammonium bicarbonate, adjusted to pH 7 with CO₂ (s)]) to give the title compound (11.6 mg, 0.029 mmol, 73.9% yield). ¹H NMR (500 MHz, DMSO-d₆) δ ppm 9.05 (s, 1H), 8.11 (s, 2H), 6.46 (d, J=1.4 Hz, 1H), 3.99-3.90 (m, 2H), 3.00 (d, J=7.3 Hz, 2H), 2.93 (d, J=6.9 Hz, 2H), 2.86 (dd, J=16.4, 5.1 Hz, 1H), 2.79-2.56 (m, 3H), 2.22 (dd, J=16.4, 10.4 Hz, 1H), 2.13-2.03 (m, 2H), 2.02-1.95 (m, 2H), 1.94-1.73 (m, 4H), 1.43-1.31 (m, 1H); MS (APCI⁺) m/z 398 [M+H]⁺.

### Example 83: 5-[(7R,8R)-7-amino-1-fluoro-3,8-dihydroxy-5,6,7,8-tetrahydronaphthalen-2-yl]-1λ⁶,2,5-thiadiazolidine-1,1,3-trione (Compound 401)

### Example 83A: 1-(benzyloxy)-5-bromo-2-chloro-3-fluorobenzene

To a solution of 5-bromo-2-chloro-1,3-difluorobenzene (25 g, 110 mmol) and benzyl alcohol (12.5 g, 115 mmol) in anhydrous tetrahydrofuran (500 mL) was added sodium tert-butoxide (2 M in tetrahydrofuran, 57.7 mL, 115 mmol). After 10 minutes, potassium tert-butoxide (1 M in tetrahydrofuran, 18.7 mL, 18.7 mmol) was added, and after 10 minutes, additional potassium tert-butoxide (1 M in tetrahydrofuran, 4.07 mL, 4.07 mmol) was added. Thereafter, saturated aqueous ammonium chloride (100 mL) was added, and the mixture was extracted with ethyl acetate (300 mL). The organic phase was washed with brine (75 mL), dried over sodium sulfate, filtered, and concentrated via rotary evaporation (31 mbar. 38° C.) to afford the title compound (34.57 g, 110 mmol, 99% yield). ¹H NMR (500 MHz, CDCl₃) δ ppm 7.46-7.33 (m, 5H), 6.98 (dd, J=8.0, 2.1 Hz, 1H), 6.93 (t, J=1.9 Hz, 1H), 5.13 (s, 2H); MS (ESI⁺) m/z 356 [M+CH₃CN]⁺.

### Example 83B: 7-(benzyloxy)-6-chloro-5-fluoro-1,4-dihydro-1,4-epoxynaphthalene

A solution of 1-(benzyloxy)-5-bromo-2-chloro-3-fluorobenzene (10.0 g, 31.7 mmol), and furan (30.2 mL, 412 mmol) was cooled to 5° C. in an ice water bath and lithium diisopropylamide (19.8 mL, 39.6 mmol, 2.0 M in tetrahydrofuran/heptane/ethylbenzene) was added over 10 minutes at <15° C. After 40 minutes with cooling back to <5° C. water (50 mL) was added at <20° C. and the mixture was extracted with tert-butyl methyl ether (50 mL). The aqueous layer was further extracted with tert-butyl methyl ether (2×50 mL). The combined organic layers were washed with brine (10 mL), dried (Na₂SO₄), and concentrated to minimal volume. Heptanes (50 mL) were added, and the mixture was concentrated to minimal volume. Heptanes (100 mL) were added again, and the mixture was heated to 90° C. and a dark material remained caked to the flask. The mixture was cooled, and tert-butyl methyl ether (200 mL) was added to get material mostly solubilized. Then the mixture was stirred with silica (5 g) for 15 minutes and filtered, washing with tert-butyl methyl ether (3×10 mL), to remove the dark color. The filtrate was concentrated to minimal volume. Heptanes (50) mL) were added, the mixture was heated to 90° C. and the slurry mostly dissolved then oiled. With slow cooling, a slurry was observed at 50-55° C. but much solid stuck to the flask walls. The mixture was sonicated and stirred vigorously to break up caked solids, scraped, stirred 30 minutes, and filtered, washing with heptanes (3×10 mL). The solid was dried in a vacuum oven at 50° C. giving the title compound (7.31 g, 24.15 mmol, 76% yield). ¹H NMR (500 MHz, CDCl₃) δ ppm 7.46-7.41 (m, 1H), 7.44-7.37 (m, 2H), 7.40-7.32 (m, 1H), 7.36-7.22 (m, 1H), 7.04 (dd, J=5.5, 1.8 Hz, 1H), 7.00 (dd, J=5.5, 1.9 Hz, 1H), 6.84 (s, 1H), 5.95 (dt, J=1.7, 0.8 Hz, 1H), 5.67 (ddd, J=2.7, 1.8, 0.9 Hz, 1H), 5.13 (d, J=1.2 Hz, 2H); MS (APCI+) m/z 303 [M+H]+.

### Example 83C: (1R,2R)-6-(benzyloxy)-7-chloro-2-(dibenzylamino)-8-fluoro-1,2-dihydronaphthalen-1-ol

A solution of 7-(benzyloxy)-6-chloro-5-fluoro-1,4-dihydro-1,4-epoxynaphthalene (7.00 g, 23.12 mmol), ammonium tetrafluoroborate (2.42 g, 23.1 mmol), tetrahydrofuran (21 mL), and dibenzylamine (5.78 mL, 30.1 mmol) was stirred at ambient temperature while N₂ sparged for 10 minutes. Then, (R)-1-[(Sp)-2-(diphenylphosphino)ferrocenyl]ethyldi-tert-butylphosphine (0.151 g, 0.277 mmol) and bis(1,5-cyclooctadiene)rhodium(I) trifluoromethanesulfonate (0.108 g, 0.231 mmol) were added, and the mixture was heated to 60° C. After 2 hours, the mixture was heated to 75° C. while allowing tetrahydrofuran to escape with an N₂ headspace flush that was continued for 1 hour. The mixture was cooled and diluted with tert-butyl methyl ether (70 mL) and water (35 mL). The layers were separated, and the organic layer was washed with brine (14 mL). The aqueous layer was extracted with tert-butyl methyl ether (70 mL). The combined organic fractions were washed with brine (14 mL), dried (Na₂SO₄), and concentrated. The residue was purified by chromatography on silica gel (10-50% tert-butyl methyl ether/heptane gradient elution with a second identical column for separation of mixed fractions, the desired regioisomer was the second eluting of the two), giving the title compound (5.79 g, 11.58 mmol, 50% yield). ¹H NMR (600 MHZ, CDCl₃) δ ppm 7.45 (dtd, J=6.9, 1.4, 0.7 Hz, 2H), 7.42-7.37 (m, 2H), 7.37-7.18 (m, 11H), 6.65-6.57 (m, 2H), 6.09 (ddd, J=9.7, 5.1, 1.0 Hz, 1H), 5.29 (s, 1H), 5.16 (s, 2H), 3.63 (dt, J=5.1, 1.7 Hz, 1H), 3.56 (d, J=13.7 Hz, 2H), 3.45 (d, J=13.7 Hz, 2H); MS (APCI⁺) m/z 500 [M+H]⁺; ee=95.4% (major 5.8 minutes, minor 6.8 minutes on CHIRALCEL®; OD-H column, 5-50% CH₃OH/CO₂ gradient, 3 mL/minute, backpressure 150 bar, column size 4.6×100 mm ID, 5 micron).

The opposite enantiomer of this material was prepared using the same method with opposite ligand enantiomer for chiral SFC method confirmation. The ee (same method with opposite major enantiomer) was 95.3% (minor at 5.72 minutes, major at 6.65 minutes).

### Example 83D: (1R,2R)-6-(benzyloxy)-7-chloro-2-(dibenzylamino)-8-fluoro-1,2,3,4-tetrahydronaphthalen-1-ol

A solution of (1R,2R)-6-(benzyloxy)-7-chloro-2-(dibenzylamino)-8-fluoro-1,2-dihydronaphthalen-1-ol (1.28 g, 2.56 mmol) and tetrahydrofuran (13 mL) was stirred at ambient temperature while water (13 mL), 4-methylbenzenesulfonohydrazide (2.38 g, 12.8 mmol), and sodium acetate (2.10 g, 25.6 mmol) were added. The mixture was heated to 60° C. and a biphasic mixture was observed. After 15 hours, the mixture was cooled and partitioned between tert-butyl methyl ether (50 mL) and 4 N NaOH (13 mL). The organic fraction was then washed with brine (5 mL), dried (Na₂SO₄), and concentrated. The residue was purified by chromatography on silica gel (25-100% dichloromethane/heptane gradient with 0.1% triethylamine), giving the title compound (1.09 g, 2.17 mmol, 85% yield). ¹H NMR (500 MHZ, CDCl₃) δ ppm 7.44-7.33 (m, 5H), 7.35-7.30 (m, 5H), 7.33-7.27 (m, 3H), 7.27-7.20 (m, 2H), 6.45 (d, J=1.6 Hz, 1H), 5.10 (s, 2H), 4.97 (d, J=8.8 Hz, 1H), 3.92 (d, J=13.5 Hz, 2H), 3.48 (d, J=13.4 Hz, 2H), 3.07 (d, J=1.3 Hz, 1H), 2.85 (ddd, J=12.5, 8.8, 2.8 Hz, 1H), 2.82-2.75 (m, 1H), 2.78-2.68 (m, 1H), 2.13 (ddt, J=12.7, 4.5, 2.9 Hz, 1H), 1.61 (tdd, J=1λ⁶, 11.5, 5.4 Hz, 1H); MS (ESI⁺) m/z 502 [M+H]⁺; chiral SFC (CHIRALCEL® OD-H column, 5-50% CH₃OH/CO₂ gradient 3 mL/minute, backpressure 150 bar, column size 4.6×100 mm ID, 5 micron) suggested 97% ee: 6.26 minutes (major) and 7.12 minutes (minor).

### Example 83E: tert-butyl {[(7R,8R)-3-(benzyloxy)-7-(dibenzylamino)-1-fluoro-8-hydroxy-5,6,7,8-tetrahydronaphthalen-2-yl]amino}acetate

A 50 mL round bottom flask was charged with 2-methyl-2-butanol (5.52 mL), and the solvent was degassed via sub-surface nitrogen sparging for 15 minutes. Thereafter, sodium tert-butoxide (10.3 mg, 0.108 mmol), tris(dibenzylideneacetone)dipalladium(0)) (0.049 g, 0.054 mmol), and RockPhos (0.055 g, 0.118 mmol) were added, and the resulting mixture was heated to an internal temperature of 80° C. After 30 minutes, the homogeneous solution was cooled to ambient temperature.

To a solution of 2-methyl-2-butanol (11.0 mL) and (1R,2R)-6-(benzyloxy)-7-chloro-2-(dibenzylamino)-8-fluoro-1,2,3,4-tetrahydronaphthalen-1-ol (1.08 g, 2.151 mmol) was added sodium trifluoroacetate (0.351 g, 2.58 mmol), tert-butyl 2-aminoacetate (0.353 mL, 2.58 mmol) and 1,5,7-triazabicyclo[4.4.0]dec-5-ene (TBD) (0.359 g, 2.58 mmol), and the solution was degassed via sub-surface nitrogen sparging for 20 minutes. Subsequently, the catalyst solution prepared above was added via syringe pump over 10 hours while the reaction was heated to an internal temperature of 70° C. The mixture was then cooled and partitioned between tert-butyl methyl ether (100 mL) and water (25 mL). The aqueous layer was back-extracted with tert-butyl methyl ether (25 mL). The combined organic extracts were washed with brine (10 mL), dried over sodium sulfate, filtered, and concentrated. The residue was purified by flash chromatography on silica gel (0-10% tert-butyl methyl ether/dichloromethane gradient with 0.1% triethylamine with a second identical column for separation of mixed fractions) gave the title compound (406 mg, 0.680 mmol, 32% yield). ¹H NMR (500 MHZ, CDCl₃) δ ppm 7.43-7.27 (m, 13H), 7.26-7.18 (m, 2H), 6.36-6.32 (m, 1H), 5.04 (s, 2H), 4.98 (d, J=8.8 Hz, 1H), 4.42 (td, J=6.3, 2.9 Hz, 1H), 3.99 (ddd, J=17.9, 6.2, 2.0 Hz, 1H), 3.95-3.86 (m, 3H), 3.48 (d, J=13.5 Hz, 2H), 3.07 (d, J=1.4 Hz, 1H), 2.84 (ddd, J=12.5, 8.8, 2.7 Hz, 1H), 2.69 (dd, J=8.3, 3.6 Hz, 2H), 2.10 (dq, J=12.4, 3.5 Hz, 1H), 1.58 (tt, J=1λ⁶, 8.6 Hz, 1H), 1.42 (s, 9H); MS (APCI⁺) m/z 597 [M+H]⁺.

### Example 83F: tert-butyl {[(7R,8R)-3-(benzyloxy)-7-(dibenzylamino)-1-fluoro-8-hydroxy-5,6,7,8-tetrahydronaphthalen-2-yl]({[(prop-2-en-1-yl)oxy]carbonyl}sulfamoyl)amino}acetate

A solution of dichloromethane (0.5 mL) and chlorosulfonyl isocyanate (0.088 mL, 1.02 mmol) was cooled to <0° C., and allyl alcohol (0.069 mL, 1.02 mmol) was added at a rate such that the internal temperature did not exceed 0° C. After 10 minutes, a preformed solution of tert-butyl {[(7R,8R)-3-(benzyloxy)-7-(dibenzylamino)-1-fluoro-8-hydroxy-5,6,7,8-tetrahydronaphthalen-2-yl]amino}acetate (405 mg, 0.679 mmol) and N,N-diisopropylethylamine (0.237 mL, 1.35 mmol) in dichloromethane (4 mL) was added at a rate such that the internal temperature did not exceed 0° C. The flask originally containing the substrate mixture was rinsed with dichloromethane (0.5+0.2 mL). After 5 minutes, the reaction was quenched with water (3 mL), and the mixture was extracted with dichloromethane (10 mL). The organic layer was washed with brine (2 mL), dried over sodium sulfate, filtered, and concentrated. The residue was purified by flash column chromatography on silica gel (0-5% tert-butyl methyl ether/dichloromethane gradient with 0.1% triethylamine) gave the title compound (308 mg, 0.405 mmol, 59.7% yield). MS (APCI⁺) m/z 760 [M+H]⁺.

### Example 83G: 5-[(7R,8R)-3-(benzyloxy)-7-(dibenzylamino)-1-fluoro-8-hydroxy-5,6,7,8-tetrahydronaphthalen-2-yl]-1λ⁶,2,5-thiadiazolidine-1,1,3-trione

The headspace of a 4 mL vial containing tert-butyl {[(7R,8R)-3-(benzyloxy)-7-(dibenzylamino)-1-fluoro-8-hydroxy-5,6,7,8-tetrahydronaphthalen-2-yl]({[(prop-2-en-1-yl)oxy]carbonyl}sulfamoyl)amino}acetate (308 mg, 0.405 mmol) was flushed with nitrogen for 5 minutes, and then added anhydrous methanol (3.1 mL) was added. The resulting mixture was degassed via sub-surface nitrogen sparging for 5 minutes. Thereafter, a solution of sodium tert-butoxide (2 M in tetrahydrofuran, 0.608 mL, 1.22 mmol) was added, and the mixture was degassed via sub-surface nitrogen sparging for 10 minutes. Then added tetrakis(triphenylphosphine)palladium(0) (3.28 mg, 2.84 μmol) was added, and the reaction was sparged for an additional 5 minutes before being heated to 50° C. After 2 hours, the reaction mixture was cooled in an ice bath and hydrochloric acid (3 M in cyclopropyl methyl ether, 0.432 mL, 1.297 mmol) was added in one portion at <10° C. and a slurry slowly developed. The mixture was stirred for 5 minutes, diluted with ethyl acetate (5 mL), and stirred for an additional 10 minutes. The solid was collected by filtration and washed with ethyl acetate (3×1 mL). The solid was dried in a vacuum oven at 50° C. giving the title compound (158 mg, 0.263 mmol, 64.8% yield). ¹H NMR (600 MHZ, DMSO-d₆/D₂O/pyridine-d₅) δ ppm 8.62 (s, 1H), 7.59-7.54 (m, 2H), 7.45-7.33 (m, 7H), 7.35-7.29 (m, 4H), 7.26-7.20 (m, 2H), 6.72 (s, 1H), 5.21-5.13 (m, 2H), 4.99 (d, J=6.0 Hz, 1H), 4.07 (d, J=2.4 Hz, 2H), 3.86 (d, J=14.3 Hz, 2H), 3.60 (d, J=14.3 Hz, 2H), 2.95 (ddd, J=9.9, 6.0, 3.5 Hz, 1H), 2.78 (dt, J=16.5, 4.9 Hz, 1H), 2.62 (dd, J=10.6, 5.0 Hz, 1H), 2.09 (dq, J=13.2, 4.5 Hz, 1H), 1.75-1.65 (m, 1H); MS (APCI+) m/z 602 [M+H]⁺.

### Example 83H: 5-[(7R,8R)-7-amino-1-fluoro-3,8-dihydroxy-5,6,7,8-tetrahydronaphthalen-2-yl]-1λ⁶,2,5-thiadiazolidine-1,1,3-trione

A solution of 5-[(7R,8R)-3-(benzyloxy)-7-(dibenzylamino)-1-fluoro-8-hydroxy-5,6,7,8-tetrahydronaphthalen-2-yl]-1λ⁶,2,5-thiadiazolidine-1,1,3-trione (100 mg, 0.166 mmol), tetrahydrofuran (6 mL), and water (2.00 mL) was added to 5% Pd/C (wet JM #9) (200 mg, 0.876 mmol) in a 20 mL Barnstead Hast C reactor, and the mixture was stirred for 2.5 hours under hydrogen (19 psi) at 25° C. The mixture was filtered, and the filtrate was concentrated. The residue was dissolved in methanol (1 mL), then ethyl acetate (5 mL) was added with sonication and a slurry developed. The mixture was stirred for 15 minutes and filtered, washing with ethyl acetate (2×2 mL), giving the title compound (11.7 mg). The catalyst was washed with additional tetrahydrofuran/water (20 mL), and the filtrate was concentrated. The residue was dissolved in methanol (10 mL), and the mixture was filtered through diatomaceous earth, washing with methanol (2×10 mL). The filtrate and washes were then concentrated. Methanol (1 mL) was added to the residue, and then ethyl acetate (10 mL) was added with stirring. The mixture was stirred for 15 minutes and filtered, washing with ethyl acetate (2×2 mL), giving additional title compound (33.0 mg). The crops were combined to give the title compound (44.7 mg, 0.135 mmol, 81% yield). ¹H NMR (600 MHZ, DMSO-d₆) δ ppm 6.45 (s, 1H), 5.45 (d, J=5.6 Hz, 1H), 4.59 (d, J=4.9 Hz, 1H), 3.98-3.89 (m, 2H), 2.74-2.60 (m, 2H), 2.07 (dddd, J=13.0, 9.3, 6.0, 3.0 Hz, 1H), 1.70 (dq, J=12.0, 5.7 Hz, 1H); MS (ESI⁻) m/z 330 [M−H]⁻.

### Example 84: N-[(2R)-8-fluoro-6-hydroxy-7-(1,1,4-trioxo-1λ⁶,2,5-thiadiazolidin-2-yl)-1,2,3,4-tetrahydronaphthalen-2-yl]acetamide (Compound 402)

### Example 84A: N-[(2R)-6-(benzyloxy)-8-fluoro-7-(1,1,4-trioxo-1λ⁶,2,5-thiadiazolidin-2-yl)-1,2,3,4-tetrahydronaphthalen-2-yl]acetamide

To a suspension of the product of Example 20G (100 mg, 0.247 mmol) and triethylamine (125 mg, 1.233 mmol) in tetrahydrofuran-dichloromethane (2:1 ratio, 1.5 mL) at 23° C. was added acetic anhydride (50.4 mg, 0.493 mmol) to give a solution. The mixture was stirred 23° C. for 0.5 hour before it was diluted with ethyl acetate (40 mL), washed with 0.2 N aqueous HCl (10 mL) and brine, dried over Na₂SO₄, and concentrated in vacuo to give the title compound as a triethylamine salt (120 mg, 0.219 mmol, 89% yield), which was used in the next step without further purification. MS (APCI⁺) m/z 448.3 [M+H]⁺.

### Example 84B: N-[(2R)-8-fluoro-6-hydroxy-7-(1,1,4-trioxo-1λ⁶,2,5-thiadiazolidin-2-yl)-1,2,3,4-tetrahydronaphthalen-2-yl]acetamide

A suspension of the product of Example 84A (60 mg, 0.134 mmol), 10% Pd—C (28.5 mg, 0.027 mmol) and ammonium formate (169 mg, 2.68 mmol) was stirred at 70° C. for 2 hours. The mixture was filtered through a plug of diatomaceous earth and the solid residue was washed with methanol. The filtrate was concentrated and purified on preparative HPLC on a Phenomenex® Luna® 10 μm C18 column (30 mm×250 mm) eluting with a gradient of acetonitrile (A) and water (B) with 0.1% trifluoroacetic acid at a flow rate of 50 mL/minute (0-1 minute 5% A, 1-20 minutes linear gradient 5-35%) to give the title compound with some impurities. The product was purified again using the same HPLC conditions to give the pure title compound (28 mg, 58% yield). ¹H NMR (500 MHZ, DMSO-d₆) δ ppm 9.99 (s, 1H), 7.93 (d, J=7.2 Hz, 1H), 6.48 (s, 1H), 4.33 (s, 2H), 3.89 (m, 1H), 2.84 (dd, J=16.3, 5.6 Hz, 1H), 2.81-2.66 (m, 2H), 2.35 (dd, J=16.5, 8.8 Hz, 1H), 1.86 (m, 1H), 1.82 (s, 3H), 1.64-1.53 (m, 1H); MS (APCI⁺) m/z 358.2 [M+H]⁺.

### Example 85: 5-(1-fluoro-3-hydroxy-7-{[(2-hydroxyethyl)amino]methyl}-5,6,7,8-tetrahydronaphthalen-2-yl)-1λ⁶,2,5-thiadiazolidine-1,1,3-trione (Compound 403)

### Example 85A: 5-[3-(benzyloxy)-7-({[2-(benzyloxy)ethyl]amino}methyl)-1-fluoro-5,6,7,8-tetrahydronaphthalen-2-yl]-1λ⁶,2,5-thiadiazolidine-1,1,3-trione

Triethylamine (0.050 mL, 0.359 mmol) was added to the product of Example 15J (41.1 mg, 0.077 mmol) in dichloromethane (1 mL) and ethanol (2 mL). The reaction mixture stirred at ambient temperature for 10 minutes. Then 2-(benzyloxy)acetaldehyde (0.056 mL, 0.396 mmol) dissolved in dichloromethane (0.25 mL) and ethanol (0.5 mL) was added, and then the mixture was stirred for an additional 2 hours. Sodium tetrahydroborate (23.30 mg, 0.616 mmol) was then added, and the resultant mixture was stirred for 3 days at ambient temperature. The reaction was quenched with 1 M HCl (0.5 mL), and the mixture was concentrated under reduced pressure. The residue was filtered through a glass microfiber frit that was rinsed with a minimal amount of methanol/N,N-dimethylformamide. The resulting filtrate was purified by reverse-phase HPLC [Waters XBridge™ RP 18 column, 5 μm, 30 mm×100 mm, flow rate 40) mL/minute, 5-100% gradient of acetonitrile in buffer 0.025 M aqueous ammonium bicarbonate, adjusted to pH 10 with ammonium hydroxide)] to give the title compound (10.8 mg, 0.020 mmol, 25.3% yield). MS (ESI⁺) m/z 554 [M+H]⁺.

### Example 85B: 5-(1-fluoro-3-hydroxy-7-{[(2-hydroxyethyl)amino]methyl}-5,6,7,8-tetrahydronaphthalen-2-yl)-1λ⁶,2,5-thiadiazolidine-1,1,3-trione

Trichloroborane (1.0 M in dichloromethane) (0.147 mL, 0.147 mmol) was added to a vial containing a suspension of the product of Example 85A (10.2 mg, 0.018 mmol) and 1,2,3,4,5-pentamethylbenzene (8 mg, 0.054 mmol) in dichloromethane (2 mL) cooled to −78° C. The mixture was stirred at −78° C. for 15 minutes, and then at 0° C. for 45 minutes. The reaction mixture was recooled to −78° C. and the reaction was quenched with the successive addition of ethyl acetate (2 mL) and ethanol (2 mL). The mixture was then allowed to warm to ambient temperature and stirred further for 15 minutes. The mixture was concentrated under reduced pressure, and the residue was filtered through a glass microfiber frit that was rinsed with a minimal amount of methanol/N,N-dimethylformamide. The resulting filtrate was purified by reverse-phase HPLC [Waters XBridge™ RP 18 column, 5 μm, 30 mm×100 mm, flow rate 40 mL/minute, 3-100% gradient of acetonitrile in buffer 0.025 M aqueous ammonium bicarbonate, adjusted to pH 10 with ammonium hydroxide)] to afford the title compound (3 mg, 8.03 μmol, 43.6% yield). ¹H NMR (400 MHZ, DMSO-d₆) δ ppm 6.44 (s, 1H), 5.11 (s, 1H), 3.93 (s, 2H), 3.65 (t, J=5.4 Hz, 2H), 2.97 (t, J=5.4 Hz, 2H), 2.92 (d, J=6.8 Hz, 2H), 2.84 (dd, J=16.6, 5.0 Hz, 1H), 2.77-2.59 (m, 2H), 2.26-2.15 (m, 1H), 2.01-1.95 (m, 1H), 1.90 (d, J=12.2 Hz, 1H), 1.42-1.28 (m, 1H); MS (ESI⁺) m/z 374 [M+H]⁺.

### Example 86: 5-[(7S)-7-(aminomethyl)-1-fluoro-3-hydroxy-5,6,7,8-tetrahydronaphthalen-2-yl]-1λ⁶,2,5-thiadiazolidine-1,1,3-trione (Compound 404)

### Example 86A: tert-butyl {[(2S)-6-(benzyloxy)-8-fluoro-7-(1,1,4-trioxo-1λ⁶,2,5-thiadiazolidin-2-yl)-1,2,3,4-tetrahydronaphthalen-2-yl]methyl}carbamate

The product of Example 15I was separated by preparative chiral SFC. Preparative SFC was performed on a Waters SFC80Q SFC running under ChromScope™ software control. The preparative SFC system was equipped with a CO₂ pump, modifier pump with 4-port solvent selection valve, automated back pressure regulator (ABPR), UV detector, and 6-position fraction collector. The mobile phase was comprised of supercritical CO₂ supplied by a Dewar of bone-dry non-certified CO₂ pressurized to 350 psi with a modifier of methanol (0.1% diethylamine) at a flow rate of 80 g/minute. The column was held at ambient temperature and the backpressure regulator was set to maintain 120 bar. The sample was dissolved in dichloromethane:methanol; acetonitrile 8:1:1 at a concentration of 41.5 mg/mL. The sample was loaded into the modifier stream in 0.5 mL injections. The mobile phase was held isocratically at 40% Cosolvent:CO₂. The instrument was fitted with a CHIRALPAK® IC column with dimensions 30 mm i.d.×250 mm length with 5 μm particles. The later eluting fraction gave the title compound (absolute stereochemistry was arbitrarily assigned). MS (ESI⁻) m/z 518 [M−H]⁻.

### Example 86B: (S)-tert-butyl ((7-(1,1-dioxido-4-oxo-1,2,5-thiadiazolidin-2-yl)-8-fluoro-6-hydroxy-1,2,3,4-tetrahydronaphthalen-2-yl)methyl)carbamate

The product of Example 86A (33 mg, 0.064 mmol), ammonium formate (32.0 mg, 0.508 mmol), and 10% Pd/C (6.7 mg, 6.30 μmol) in ethanol (3 mL) was heated to 65° C. for 1.5 hours. The reaction mixture was cooled to ambient temperature and filtered over diatomaceous earth that was rinsed with methanol. The filtrate was concentrated under reduced pressure and purified by reverse phase column chromatography (30 g Biotage®; Sfär C18 Duo 100 Å 30 μm column, 10 to 100% methanol in water [buffered with 0.025 M aqueous ammonium bicarbonate, adjusted to pH 7 with CO₂(s)], flow rate=25 mL/minute) to give the title compound as the ammonium salt (24 mg, 0.054 mmol, 85% yield). MS (ESI⁻) m/z 428 [M−H]⁻.

### Example 86: 5-[(7S)-7-(aminomethyl)-1-fluoro-3-hydroxy-5,6,7,8-tetrahydronaphthalen-2-yl]-1λ⁶,2,5-thiadiazolidine-1,1,3-trione

2,2,2-Trifluoroacetic acid (200 μL, 2.60 mmol) was added to a solution of the product of Example 86B (24 mg, 0.056 mmol) in dichloromethane (2 mL), and the mixture was stirred at ambient temperature for 1.5 hours. The reaction mixture was concentrated under reduced pressure, and the residue was azeotroped with toluene (3× 2 mL). The residue was triturated with acetonitrile and filtered to give the title compound as a trifluoracetic acid salt (14.1 mg, 0.032 mmol, 56.9% yield). ¹H NMR (400 MHZ, DMSO-d₆) δ ppm 9.03 (s, 1H), 7.78 (s, 3H), 6.44 (d, J=1.4 Hz, 1H), 3.93 (d, J=1.1 Hz, 2H), 2.91-2.79 (m, 3H), 2.78-2.57 (m, 2H), 2.20 (dd, J=16.5, 10.5 Hz, 1H), 1.94-1.84 (m, 2H), 1.41-1.27 (m, 1H); MS (ESI⁺) m/z 330 [M+H]⁺.

### Example 87: 5-[(7R)-7-(aminomethyl)-1-fluoro-3-hydroxy-5,6,7,8-tetrahydronaphthalen-2-yl]-1λ⁶,2,5-thiadiazolidine-1,1,3-trione (Compound 405)

### Example 87A: tert-butyl {[(2R)-6-(benzyloxy)-8-fluoro-7-(1,1,4-trioxo-1λ⁶,2,5-thiadiazolidin-2-yl)-1,2,3,4-tetrahydronaphthalen-2-yl]methyl}carbamate

The product of Example 15I was separated by preparative chiral SFC. Preparative SFC was performed on the Waters SFC80Q SFC running under ChromScope™ software control. The preparative SFC system was equipped with a CO₂ pump, modifier pump with 4-port solvent selection valve, automated back pressure regulator (ABPR), UV detector, and 6-position fraction collector. The mobile phase was comprised of supercritical CO₂ supplied by a Dewar of bone-dry non-certified CO₂ pressurized to 350 psi with a modifier of methanol (0.1% diethylamine) at a flow rate of 80 g/minute. The column was held at ambient temperature and the backpressure regulator was set to maintain 120 bar. The sample was dissolved in dichloromethane:methanol; acetonitrile 8:1:1 at a concentration of 41.5 mg/mL. The sample was loaded into the modifier stream in 0.5 mL injections. The mobile phase was held isocratically at 40% Cosolvent:CO₂. The instrument was fitted with a CHIRALPAK® IC column with dimensions 30 mm i.d.×250 mm length with 5 μm particles. The earlier eluting fraction gave the title compound (absolute stereochemistry was arbitrarily assigned). MS (ESI⁻) m/z 518 [M−H]⁻.

### Example 87B: tert-butyl {[(2R)-8-fluoro-6-hydroxy-7-(1,1,4-trioxo-1λ⁶,2,5-thiadiazolidin-2-yl)-1,2,3,4-tetrahydronaphthalen-2-yl]methyl}carbamate

The product of Example 87A (55.6 mg, 0.107 mmol), ammonium formate (54.0 mg, 0.856 mmol), and 10% Pd/C (11 mg, 10.34 μmol) in ethanol (3 mL) was heated to 65° C. for 1 hour and 15 minutes. The reaction mixture was cooled to ambient temperature and filtered over diatomaceous earth that was rinsed with methanol. The filtrate was concentrated under reduced pressure. The residue was purified by reverse phase column chromatography (30 g Biotage®) Sfär C18 Duo 100 Å 30 μm column, 10 to 100% methanol in water [buffered with 0.025 M aqueous ammonium bicarbonate, adjusted to pH 7 with CO₂ (s)], flow rate=25 mL/minute) to give the title compound as an ammonium salt (38.9 mg, 0.087 mmol, 81% yield). MS (APCI⁻) m/z 428 [M−H]⁻.

### Example 87C: 5-[(7R)-7-(aminomethyl)-1-fluoro-3-hydroxy-5,6,7,8-tetrahydronaphthalen-2-yl]-1λ⁶,2,5-thiadiazolidine-1,1,3-trione

2,2,2-Trifluoroacetic acid (200 μL, 2.60 mmol) was added to a solution of the product of Example 87B (38.9 mg, 0.091 mmol) in dichloromethane (2 mL), and the mixture was stirred at ambient temperature for 2 hours. The reaction mixture was concentrated under reduced pressure, and the residue was azeotroped with toluene (3×2 mL). The residue was triturated with acetonitrile and filtered to give the title compound as a trifluoroacetic acid salt (22.1 mg, 0.050 mmol, 55.0% yield). ¹H NMR (600 MHZ, DMSO-d₆) δ ppm 9.03 (s, 1H), 7.78 (s, 3H), 6.44 (s, 1H), 3.93 (d, J=2.3 Hz, 2H), 2.88-2.79 (m, 3H), 2.77-2.70 (m, 1H), 2.68-2.60 (m, 1H), 2.20 (dd, J=16.5, 10.5 Hz, 1H), 1.95-1.86 (m, 2H), 1.39-1.29 (m, 1H); MS (ESI⁺) m/z 330 [M+H]⁺.

### Example 88: 5-{(7R,8R)-1-fluoro-3,8-dihydroxy-7-[(3-methylbutyl)amino]-5,6,7,8-tetrahydronaphthalen-2-yl}-1λ⁶,2,5-thiadiazolidine-1,1,3-trione (Compound 406)

A solution of isovaleraldehyde (6.50 μL, 0.060 mmol) in methanol (1 mL) was charged to a 4 mL vial containing the product of Example 83H (20 mg, 0.060 mmol). After 5 minutes at ambient temperature, sodium cyanoborohydride (3.8 mg, 0.060 mmol) was added, and the mixture was stirred for 10 minutes. The mixture was directly purified by silica gel flash chromatography [5×4 g Teledyne ISCO RediSep Rf Gold® silica gel columns, serial coupled, flow rate 20 mL/minute, A; acetonitrile/deionized water (97:3); B: 0.1% trifluoroacetic acid in deionized water; gradient: 3% B (0-1 minute), 3-20% B (1-9 minutes)] to give the title compound (10 mg, 0.025 mmol, 41% yield) as a trifluoroacetic acid salt. ¹H NMR (500 MHZ, DMSO-d₆) δ ppm 9.77 (s, 1H), 8.50 (s, 1H), 8.26 (s, 1H), 6.50 (d, J=1.2 Hz, 1H), 4.84 (d, J=4.4 Hz, 1H), 4.11 (d, J=13.9 Hz, 1H), 4.06 (d, J=13.9 Hz, 1H), 3.09-3.00 (m, 2H), 2.71 (dddd, J=21.1, 17.5, 12.3, 4.7 Hz, 2H), 2.22-2.12 (m, 1H), 1.86 (dq, J=13.2, 6.4 Hz, 1H), 1.63 (dp, J=13.2, 6.6 Hz, 1H), 1.51 (tdd, J=12.7, 10.9, 6.4 Hz, 2H), 0.90 (d, J=6.6 Hz, 6H); MS (ESI+) m/z 402 [M+H]⁺.

### Example 89: 5-[(2S)-2-(aminomethyl)-4-fluoro-6-hydroxy-2,3-dihydro-1H-inden-5-yl]-1λ⁶,2,5-thiadiazolidine-1,1,3-trione (Compound 407)

### Example 89A: tert-butyl {[4-fluoro-6-hydroxy-5-(1,1,4-trioxo-1λ⁶,2,5-thiadiazolidin-2-yl)-2,3-dihydro-1H-inden-2-yl]methyl}carbamate, ammonium salt

Triethylamine (0.091 mL, 0.655 mmol) was added to the product of Example 7L (70.3 mg, 0.164 mmol) in acetonitrile (1.5 mL) and stirred at ambient temperature for 10 minutes. Then di-tert-butyl dicarbonate (0.04 mL, 0.174 mmol) was added, and the mixture was stirred for an additional 2 hours. The reaction mixture was concentrated under reduced pressure, and the residue was purified by flash column chromatography (30 g Biotage® Sfär C18 Duo 100 Å 30 μm column, 10 to 100% methanol in water [buffered with 0.025 M aqueous ammonium bicarbonate, adjusted to pH 7 with CO₂ (s)], flow rate=25 mL/minute) to give the title compound as an ammonium salt (67.7 mg, 0.157 mmol, 96% yield). MS (ESI⁺) m/z 433 [M+NH₄]⁺.

### Example 89B: tert-butyl {[(2S)-4-fluoro-6-hydroxy-5-(1,1,4-trioxo-1λ⁶,2,5-thiadiazolidin-2-yl)-2,3-dihydro-1H-inden-2-yl]methyl}carbamate

The product of Example 89A was separated by preparative chiral SFC. Preparative SFC was performed on the Waters SFC80Q SFC running under ChromScope™ software control. The preparative SFC system was equipped with a CO₂ pump, modifier pump with 4-port solvent selection valve, automated back pressure regulator (ABPR), UV detector, and 6-position fraction collector. The mobile phase was comprised of supercritical CO₂ supplied by a Dewar of bone-dry non-certified CO₂ pressurized to 350 psi with a modifier of methanol (0.1% triethylamine) at a flow rate of 80 g/minute. The column was held at ambient temperature and the backpressure regulator was set to maintain 120 bar. The sample was dissolved in methanol:dimethyl sulfoxide 1:1 at a concentration of 15.32 mg/mL. The sample was loaded into the modifier stream in 0.25 mL injections. The mobile phase was held isocratically at 40% Cosolvent:CO₂. The instrument was fitted with a CHIRALPAK® IC column with dimensions 30 mm i.d.×250 mm length with 5 μm particles. The later eluting fraction gave the title compound (absolute stereochemistry was arbitrarily assigned). MS (ESI⁻) m/z 414 [M−H]⁻.

### Example 89C: 5-[(2S)-2-(aminomethyl)-4-fluoro-6-hydroxy-2,3-dihydro-1H-inden-5-yl]-1λ⁶,2,5-thiadiazolidine-1,1,3-trione

2,2,2-Trifluoroacetic acid (200 μL, 2.60 mmol) was added to a solution of the product of Example 89B (11.1 mg, 0.027 mmol) in dichloromethane (2 mL), and the mixture was stirred at ambient temperature for 1 hour. The reaction mixture was concentrated under reduced pressure, and the residue was azeotroped with toluene (3×2 mL). The residue was triturated with acetonitrile and filtered to give the title compound as a trifluoroacetic acid salt (5.6 mg, 0.013 mmol, 48.8% yield). ¹H NMR (500 MHZ, DMSO-d₆) δ ppm 9.03 (s, 1H), 7.75 (s, 3H), 6.57 (s, 1H), 3.92 (s, 2H), 3.03-2.95 (m, 2H), 2.94-2.88 (m, 2H), 2.73-2.60 (m, 3H); MS (ESI⁻) m/z 314 [M−H]⁻.

### Example 90: 5-[(2R)-2-(aminomethyl)-4-fluoro-6-hydroxy-2,3-dihydro-1H-inden-5-yl]-1λ⁶,2,5-thiadiazolidine-1,1,3-trione (Compound 408)

### Example 90A: tert-butyl {[(2R)-4-fluoro-6-hydroxy-5-(1,1,4-trioxo-1λ⁶,2,5-thiadiazolidin-2-yl)-2,3-dihydro-1H-inden-2-yl]methyl}carbamate

The product of Example 89A was separated by preparative chiral SFC. Preparative SFC was performed on the Waters SFC80Q SFC running under ChromScope™ software control. The preparative SFC system was equipped with a CO₂ pump, modifier pump with 4-port solvent selection valve, automated back pressure regulator (ABPR), UV detector, and 6-position fraction collector. The mobile phase was comprised of supercritical CO₂ supplied by a Dewar of bone-dry non-certified CO₂ pressurized to 350 psi with a modifier of methanol (0.1% triethylamine) at a flow rate of 80 g/minute. The column was held at ambient temperature and the backpressure regulator was set to maintain 120 bar. The sample was dissolved in methanol:dimethyl sulfoxide 1:1 at a concentration of 15.32 mg/mL. The sample was loaded into the modifier stream in 0.25 mL injections. The mobile phase was held isocratically at 40% Cosolvent:CO₂. The instrument was fitted with a CHIRALPAK® IC column with dimensions 30 mm i.d.×250 mm length with 5 μm particles. The earlier eluting enantiomer peak gave the title compound (absolute stereochemistry was arbitrarily assigned). MS (ESI⁻) m/z 414 [M−H]⁻.

### Example 90B: 5-[(2R)-2-(aminomethyl)-4-fluoro-6-hydroxy-2,3-dihydro-1H-inden-5-yl]-1λ⁶,2,5-thiadiazolidine-1,1,3-trione

2,2,2-Trifluoroacetic acid (200 μL, 2.60 mmol) was added to a solution of the product of Example 90A (12.9 mg, 0.031 mmol) in dichloromethane (2 mL), and the mixture was stirred at ambient temperature for 2 hours. The reaction mixture was concentrated under reduced pressure, and the residue was azeotroped with toluene (3×2 mL). The residue was triturated with acetonitrile and filtered to give the title compound as a trifluoroacetic acid salt (7.3 mg, 0.017 mmol, 54.8% yield). ¹H NMR (500 MHZ, DMSO-d₆) δ ppm 9.04 (s, 1H), 7.75 (s, 3H), 6.57 (s, 1H), 3.92 (s, 2H), 3.03-2.94 (m, 2H), 2.97-2.86 (m, 2H), 2.74-2.61 (m, 3H); MS (ESI⁺) m/z 316 [M+H]⁺.

### Example 91: 5-{(7R)-7-[(5-amino-4,4-difluoropentyl)amino]-1-fluoro-3-hydroxy-5,6,7,8-tetrahydronaphthalen-2-yl}-1λ⁶,2,5-thiadiazolidine-1,1,3-trione (Compound 409)

### Example 91A: tert-butyl 5-azido-4,4-difluoropentanoate

To a solution of tert-butyl 4,4-difluoro-5-[(trifluoromethanesulfonyl)oxy]pentanoate (29.2 g, 85 mmol) (prepared by the method described in European Journal of Organic Chemistry, 2015, vol. 2015, 3689-3701) in dimethyl sulfoxide (584 mL) was added sodium azide (21.6 g, 332 mmol) in portions at 20° C., and the resulting mixture was stirred at 20° C. for 12 hours. The reaction mixture was partitioned between water (2000 mL) and ethyl acetate (1000 mL). The organic phase was separated, washed with brine (4×400 mL), dried over anhydrous Na₂SO₄, filtered and concentrated under reduced pressure. The residue was purified by column chromatography on silica gel eluting with ethyl acetate in petroleum ether from 20% to 25% to afford the title compound (20 g, 80% yield, purity 80%). ¹H NMR (400 MHZ, CDCl₃) δ ppm 3.49 (t, J=12.9 Hz, 2H), 2.50-2.42 (m, 2H), 2.32-2.16 (m, 3H), 1.47-1.45 (m, 9H).

### Example 91B: tert-butyl 5-[(tert-butoxycarbonyl)amino]-4,4-difluoropentanoate

To a solution of the product of Example 91A (18 g, 61.2 mmol, purity 80%) and di-tert-butyl dicarbonate (21.32 mL, 92 mmol) in tetrahydrofuran (300 mL) at 20° C. was added a suspension of 10% Pd—C (6.51 g, 6.12 mmol) in tetrahydrofuran (60 mL), and the resulting mixture was stirred under H₂ (15 psi) at 20° C. for 12 hours. One additional reaction on 2 g scale was run as described above. The reaction mixtures were filtered through a pad of diatomaceous earth and the solid residue was washed with ethyl acetate (2×200 mL). The combined filtrates were concentrated under reduced pressure, and the residue was purified by column chromatography on silica gel eluted with ethyl acetate in petroleum ether from 5% to 10% to afford the title compound (25 g, 95% yield, purity 80%). ¹H NMR (400 MHZ, CDCl₃) δ ppm 4.83 (br s, 1H), 3.58-3.41 (m, 2H), 2.51-2.41 (m, 2H), 2.28-2.09 (m, 2H), 1.53 (s, 9H), 1.45 (s, 9H).

### Example 91C: tert-butyl 5-[bis (tert-butoxycarbonyl)amino]-4,4-difluoropentanoate

To a solution of the product of Example 91B (24 g, 62.1 mmol, purity 80%) in di-tert-butyl dicarbonate (200 mL, 861 mmol) was added 4-dimethylaminopyridine (15.16 g, 124 mmol) in portions at 20° C., and the resulting mixture was stirred at 20° C. for 12 hours. The reaction mixture was diluted with water (300 mL) and extracted with ethyl acetate (3×300 mL). The combined organic phases were washed with brine (200 mL), dried over Na₂SO₄ and concentrated under reduced pressure. The residue was purified by column chromatography on silica gel (petroleum ether:ethyl acetate=30:1) to afford the title compound (16 g, 63.0% yield, purity 70%). ¹H NMR (400 MHZ, CDCl₃) δ ppm 4.06-3.97 (m, 2H), 2.47-2.38 (m, 2H), 2.21-2.06 (m, 2H), 1.44 (d, J=0.7 Hz, 18H), 1.41 (s, 9H).

### Example 91D: di-tert-butyl (2,2-difluoro-5-hydroxypentyl)-2-imidodicarbonate

To a solution of the product of Example 91C (16 g, 39.1 mmol, purity 70%) in tetrahydrofuran (400 mL) was added 1 N diisobutylaluminum hydride-H in tetrahydrofuran (78 mL, 78 mmol) dropwise at −70° C. under N₂. After addition, the resulting mixture was slowly warmed up to 20° C. and stirred at 20° C. for 1 hour. One additional reaction on 25 g scale was run as described above. The reactions were slowly quenched with saturated aqueous NH₄Cl solution (400 mL) at 0° C. and then diluted with ethyl acetate (300 mL). The resulting mixture was filtered through a pad of diatomaceous earth, and the solid residue was washed with ethyl acetate (2×200 mL). The biphasic filtrate was separated. The organic phase was washed with brine (200 mL), dried over Na₂SO₄, and concentrated under reduced pressure. The residue was purified by column chromatography eluted with ethyl acetate in petroleum ether from 15% to 20% to afford the title compound (2.5 g, 16.97% yield, purity 90%). ¹H NMR (400 MHZ, CDCl₃) δ ppm 4.11-4.01 (m, 2H), 3.73-3.66 (m, 2H), 2.02-1.90 (m, 2H), 1.85-1.75 (m, 2H), 1.56-1.49 (m, 18H).

### Example 91E: di-tert-butyl (2,2-difluoro-5-oxopentyl)-2-imidodicarbonate

To a solution of the product of Example 91D (1 g, 2.357 mmol, purity 90%) in dichloromethane (15 mL) was added Dess-Martin periodinane (1,1,1-tris (acetyloxy)-1,1-dihydro-1,2-benzodioxol-3-(1H)-one) (1.5 g, 3.54 mmol) in portions at 0° C., and the resulting mixture was stirred at 0° C. for 2 hours. The reaction mixture was diluted with water (30 mL) and filtered through a pad of diatomaceous earth. The cake was washed with dichloromethane (2×50 mL). The resulting biphasic filtrate was separated. The organic phase was washed with brine (20 mL), dried over anhydrous Na₂SO₄, filtered, and concentrated under reduced pressure. The residue was purified by column chromatography on silica gel eluting with ethyl acetate in petroleum ether from 2% to 5% to afford the title compound (2.4 g, 87% yield, purity 80%). ¹H NMR (400 MHZ, CDCl₃) δ ppm 9.78-9.85 (m, 1H) 4.01-4.12 (m, 2H) 2.74 (t, J=7.44 Hz, 2H) 2.14-2.29 (m, 2H) 1.48-1.55 (m, 18H).

### Example 91F: di-tert-butyl (5-{[(2R)-6-(benzyloxy)-8-fluoro-7-(1,1,4-trioxo-1λ⁶,2,5-thiadiazolidin-2-yl)-1,2,3,4-tetrahydronaphthalen-2-yl]amino}-2,2-difluoropentyl)-2-imidodicarbonate

The title compound was prepared from Example 91E and Example 20G in 65% yield by the same method as described for Example 20H. MS (ESI⁻) m/z 725 [M−H]⁻.

### Example 91G: 5-{(7R)-7-[(5-amino-4,4-difluoropentyl)amino]-1-fluoro-3-hydroxy-5,6,7,8-tetrahydronaphthalen-2-yl}-1λ⁶,2,5-thiadiazolidine-1,1,3-trione

To a solution of the product of Example 91F (180 mg, 0.149 mmol, purity 60%) in methanol (20 mL) at 20° C. was added HCl (0.297 mL, 1 N aqueous) followed by 10% Pd—C (79 mg, 0.074 mmol). The resulting mixture was stirred under H₂ (15 psi) at 20° C. for 12 hours. The reaction mixture was filtered, and the solid residue was washed with methanol (2×25 mL). The filtrate was concentrated under reduced pressure. The residue was dissolved in 2 M HCl in ethyl acetate (40 mL) and stirred at 20° C. for 2 hours. The reaction mixture was then adjusted to pH=6-7 with NaHCO₃solid, filtered and concentrated under reduced pressure. The residue was purified by preparative HPLC on a Waters Xbridge™ BEH C18 100 mm×30 mm, 10 μm column with acetonitrile-10 mM NH₄HCO₃ in H₂O with a gradient 0-30% for 10 minutes and 30-100% for 3 minutes) at a flow rate of 25 mL/minute to afford the title compound (21 mg, 30.2% yield, purity 96.8%). ¹H NMR (400 MHZ, methanol-d₄) δ ppm 1.72-1.85 (m, 1H) 1.91-2.00 (m, 2H) 2.06-2.21 (m, 2H) 2.23-2.33 (m, 1H) 2.55-2.66 (m, 1H) 2.85-2.94 (m, 2H) 3.17-3.29 (m, 6H) 3.46-3.56 (m, 1H) 4.26 (s, 2H) 6.53-6.58 (m, 1H); MS (ESI⁻) m/z 435 [M−H]⁻.

### Example 92: 5-[(7R)-7-(butylamino)-1-fluoro-3-hydroxy-5,6,7,8-tetrahydronaphthalen-2-yl]-1λ⁶,2,5-thiadiazolidine-1,1,3-trione (Compound 410)

### Example 92A; benzyl [(2R)-6-(benzyloxy)-7-bromo-8-fluoro-1,2,3,4-tetrahydronaphthalen-2-yl]butylcarbamate

To a suspension of the product of Example 6B (99.5 g, 257 mmol) in a mixture of dichloromethane (600 mL), and ethanol (400 mL) was added triethylamine (36.4 g, 360 mmol) and after 3 minutes, butyraldehyde (24.1 g, 334 mmol) was added. The suspension was stirred at room temperature for 2 hours after which sodium borohydride (24.3 g, 643 mmol) was added carefully portionwise (caution: gas evolution!). After 10 minutes, the reaction was quenched via slow addition of methanol (200 mL) over 10 minutes followed by water (500 mL) over 20 minutes and dichloromethane (600 mL). The resulting biphasic suspension was filtered through a plastic fritted funnel, and the aqueous layer was extracted with dichloromethane (1×400 mL). The combined organic extracts were washed with brine (1×1 L), dried over sodium sulfate, filtered, and concentrated to afford 98.2 g of butylamine that was used in the subsequent reaction without further purification.

To a solution of the crude butylamine in a mixture of tetrahydrofuran (470 mL) and water (235 mL) was added 1 M sodium hydroxide (242 mL, 242 mmol) in one portion followed by neat benzyl chloroformate (42.5 g, 249 mmol) slowly over 5 minutes. After 10 minutes, additional benzyl chloroformate (4.14 g, 24.2 mmol) was added, and the reaction was judged to be complete. The mixture was partitioned between water (700 mL) and ethyl acetate (2×300) mL). The combined organic extracts were washed with brine (2×300 mL), dried over sodium sulfate, filtered, diluted with heptanes (400 mL), and concentrated to give 278 g of a residue. The crude residue was dissolved in toluene (500 mL) and silica gel (270 g) was added. The suspension was stirred vigorously for 20 minutes and subsequently loaded onto a bed of diatomaceous earth (250 g) topped with silica gel (400 g) that had been pre-equilibrated with heptanes (750) mL) and toluene (250 mL). The pad was flushed with toluene (800 mL) and toluene/ethyl acetate (10:1, 3×500 mL). The eluted material was concentrated to afford 239 g of a residue that was triturated with ethyl acetate/heptanes (10:1, 500 mL) over 2 hours. The solid was then collected by filtration, washed with ethyl acetate/heptanes (10:1, 1×250 mL), and dried in a vacuum oven (19 mbar, 39° C.) to afford 175.03 g of the title compound. The mother liquor was concentrated, and the residue was purified by flash chromatography on silica [330 g, heptanes to 10% acetone/heptanes] to afford an additional 42.6 g of the title compound. The two crops gave the title compound (217.6 g, 403 mmol, 90% yield over 2 steps). MS (APCI⁺) m/z 541 [M+H]⁺.

### Example 92B: tert-butyl {[(7R)-3-(benzyloxy)-7-{[(benzyloxy)carbonyl](butyl)amino}-1-fluoro-5,6,7,8-tetrahydronaphthalen-2-yl]({[(prop-2-en-1-yl)oxy]carbonyl}sulfamoyl)amino}acetate

A heat-dried two-necked 1 L round-bottomed flask was charged with 2-methyl-2-butanol (502 mL), and the solvent was degassed via sub-surface nitrogen sparging for 2 hours. Thereafter, sodium tert-butoxide (386 mg, 4.02 mmol), tetrakis(triphenylphosphine)palladium(0) (1.84 g, 2.01 mmol), and RockPhos (2.07 g, 4.42 mmol) were added, and the resulting solution was heated to an internal temperature of 80° C. After 30 minutes, the homogeneous solution was cooled to room temperature. In a separate vessel, a suspension of the product of Example 92A (217 g, 402 mmol) and sodium trifluoroacetate (65.5 g, 482 mmol) in 2-methyl-2-butanol (1.5 L) was heated to an internal temperature of 70° C., at which point a homogeneous solution was obtained. The solution was degassed via sub-surface nitrogen sparging while cooling to room temperature over 1 hour and thereafter charged with tert-butyl-2-aminoacetate (65.5 g, 482 mmol) and 1,5,7-triazabicyclo[4.4.0]dec-5-ene (TBD) (67.1 g, 482 mmol). After the solution was degassed for 1 hour, the catalyst solution prepared above was added via cannula under positive nitrogen pressure. The resulting solution was further degassed for 15 minutes and subsequently heated to an internal temperature of 70° C. After 8 hours, the mixture was cooled to room temperature and partitioned between ethyl acetate (1.5 L) and water (3 L). The aqueous layer was back-extracted with ethyl acetate (1×600 mL). The combined organic extracts were washed with 1 molar hydrochloric acid (1×1 L) and brine (2×1 L), dried over sodium sulfate, filtered, concentrated, and residual 2-methyl-2-butanol and water were evaporated via azeotropic removal with toluene (3×300 mL) to afford 295 g of crude tert-butyl {[(7R)-3-(benzyloxy)-7-{[(benzyloxy)carbonyl](butyl)amino}-1-fluoro-5,6,7,8-tetrahydronaphthalen-2-yl]amino}acetate that was immediately used in the next step without further purification.

To a solution of chlorosulfonyl isocyanate (52.3 mL, 602 mmol) in dichloromethane (1.3 L) at an internal temperature of −9° C. was added allyl alcohol (40.9 mL, 602 mmol) at a rate such that the internal temperature did not exceed 0° C. After 30 minutes, a preformed solution of crude product from above (237 g, 401 mmol based on quantitative yield from the previous step) and Hunig's base (N,N-diisopropylethylamine) (140 mL, 802 mmol) in dichloromethane (670 mL) was added via cannula at a rate such that the internal temperature did not exceed 0° C.) The flask originally containing the substrate mixture was thereafter rinsed with dichloromethane (50) mL). After 15 minutes, the reaction was quenched with water (300 mL), stirred for 5 minutes, and then the layers were separated. The organic layer was dried over sodium sulfate, filtered and concentrated. The crude residue was suspended in ethyl acetate (700 mL) and concentrated. Another portion of ethyl acetate (500 mL) was added, and the resulting slurry was vigorously stirred for 2 hours, over which time the material eventually dissolved and precipitated. After 10 hours, the suspension was filtered, and the solid was washed with ethyl acetate/heptanes (500 mL) and dried in a vacuum oven (23 mbar. 35° C.) to constant weight to afford the title compound (237.69 g, 315 mmol, 79% yield). MS (APCI⁺) m/z 755 [M+H]⁺.

### Example 920; benzyl [(2R)-6-(benzyloxy)-8-fluoro-7-(1,1,4-trioxo-1λ⁶,2,5-thiadiazolidin-2-yl)-1,2,3,4-tetrahydronaphthalen-2-yl]butylcarbamate

The headspace of a 5 L three-necked round-bottom flask containing tert-butyl {[(7R)-3-(benzyloxy)-7-{[(benzyloxy)carbonyl](butyl)amino}-1-fluoro-5,6,7,8-tetrahydronaphthalen-2-yl]({[(prop-2-en-1-yl)oxy]carbonyl}sulfamoyl)amino}acetate (207.28 g, 275 mmol) was exchanged for nitrogen. Thereafter, anhydrous methanol (917 mL) was added, and the suspension was cooled to an internal temperature of 3° C. A solution of sodium tert-butoxide (2 M in tetrahydrofuran, 376 g, 825 mmol) was added over 2 minutes, resulting in an exotherm to 24° C. After cooling to 16° C., tetrakis(triphenylphosphine)palladium(0)) (1.59 g, 1.38 mmol) was added, and the reaction was heated to an internal temperature of 50° C. After 2 hours, the mixture was cooled to an internal temperature of 2° C. and quenched with hydrochloric acid (3 M in cyclopentyl methyl ether. 262 g, 880 mmol). After 5 minutes, water (2.1 L) was added, and after stirring for 5 minutes, isopropyl acetate (1.3 L) was added, and the layers were separated. The aqueous layer was back-extracted with isopropyl acetate (650 mL), and the combined organic layers were washed with brine (800 mL), dried over sodium sulfate, filtered and concentrated. The resulting crude product was dissolved in a mixture of methanol (900 g), isopropyl acetate (1412 g) and tetrahydrofuran (444 g) and washed with aqueous hydrochloric acid (6 M, 500 mL). Before allowing layer separation, the biphasic mixture was diluted with brine (1.25 L) and water (750) mL) and agitated to achieve rapid layer separation. The organic layer was washed with brine (1.25 L), dried over sodium sulfate, filtered and concentrated to 700 g. Isopropyl acetate (600 g) was added, and the mixture was concentrated to 400 g. More isopropyl acetate (600 g) was added, and the slurry was stirred for 2 hours, then diluted with heptane (1.8 L) and stirred for 16 hours. After filtration and complete deliquoring, the filter cake was washed with isopropyl acetate/heptane (2:5, 500 mL) and dried in a vacuum oven at 24 mbar at 50° C. for 16 hours to afford the title compound (173.1 g, 280 mmol, 89% yield). MS (APCI⁺) m/z 597 [M+H]⁺.

### Example 92D: 5-[(7R)-7-(butylamino)-1-fluoro-3-hydroxy-5,6,7,8-tetrahydronaphthalen-2-yl]-1λ⁶,2,5-thiadiazolidine-1,1,3-trione, sodium salt

5% Pd/Al₂O₃ (4.15 g) was added to a suspension of benzyl [(2R)-6-(benzyloxy)-8-fluoro-7-(1,1,4-trioxo-1λ⁶,2,5-thiadiazolidin-2-yl)-1,2,3,4-tetrahydronaphthalen-2-yl]butylcarbamate (12.1 g, 19.5 mmol) in water (97 mL) and tetrahydrofuran (292 mL). The resulting suspension was stirred under hydrogen (60 psi) for 20 hours. The reactor was depressurized, and the mixture was treated with sodium hydroxide (1 M, 20.5 mL, 20.5 mmol), stirred 2 hours, diluted with water/tetrahydrofuran (1:3, 100 mL), stirred an additional 15 minutes, then filtered through a glass fiber filter which was washed with water/tetrahydrofuran (1:3, 100 mL). The combined filtrates were concentrated to 200 mL, diluted with isopropanol (300 mL), concentrated to 200 mL, diluted with isopropanol (400 mL) and concentrated to dryness. The crude residue was treated with ethyl acetate (100 mL), sonicated for 10 minutes, then filtered. The filter cake was washed with ethyl acetate (50 mL) and dried to constant mass in a vacuum oven (22 mbar) at 60° C. for 18 hours to afford the title compound (5.86 g, 14.9 mmol, 76% yield). ¹H NMR (600 MHZ, DMSO-d₆) δ ppm 6.31 (br s, 1H), 3.93 (ABq, J=7.7, 7.3 Hz, 2H), 2.80 (dd, J=16.4, 5.6 Hz, 1H), 2.71 (m, 2H), 2.57 (m, 3H), 2.16 (dd, J=15.9, 8.8 Hz, 1H), 1.87 (m, 1H), 1.36 (m, 5H), 0.87 (t, J=7.3 Hz, 3H); MS (ESI⁺) m/z 393 [M−H]⁻.

### Example 93: 5-{(6S,7S)-1-fluoro-3,6-dihydroxy-7-[(3-methylbutyl)amino]-5,6,7,8-tetrahydronaphthalen-2-yl}-1λ⁶,2,5-thiadiazolidine-1,1,3-trione (Compound 411)

### Example 93A; methyl 2-(N-((1aS,2S,7bR)-6-(benzyloxy)-2-((tert-butoxycarbonyl)amino)-4-fluoro-1a,2,3,7b-tetrahydronaphtho[1,2-b]oxiren-5-yl)-2,2,2-trifluoroacetamido)acetate

To a mixture of the product of Example 75F (3.6 g, 5.86 mmol, purity 90%), acetone (100 mL, 1362 mmol) and sodium bicarbonate (4.93 g, 58.6 mmol) in ethyl acetate (100 mL) and water (50 mL) was added a solution of OXONE® (potassium peroxomonosulfate) (10.82 g, 17.59 mmol) in water (100 mL) dropwise over 1 hour at 0-5° C. The mixture was stirred for 1.5 hours at 0-5° C. One additional reaction on 3.6 g scale was run as described above. The reaction mixtures were poured into water (1000 mL), and the mixture was extracted with ethyl acetate (3×400 mL). The organic layers were combined, washed with brine (200 mL), dried over anhydrous Na₂SO₄, and concentrated under reduced pressure to give the title compound (8 g, crude) which was used directly without further purification.

### Example 93B; methyl [{(1aS,2S,7bR)-2-[(tert-butoxycarbonyl)amino]-4-fluoro-6-hydroxy-1a,2,3,7b-tetrahydronaphtho[1,2-b]oxiren-5-yl}(trifluoroacetyl)amino]acetate

To a mixture of the product of Example 93A (8 g, crude) in tetrahydrofuran (150 mL) was added a mixture of 10% Pd—C (1 g, 0.940 mmol) in tetrahydrofuran (50 mL) at 25° C. The mixture was degassed and purged with H₂ 3 times, then the mixture was stirred under H₂ (15 psi) at 25° C. for 12 hours. The mixture was diluted with methanol (300 mL) and filtered. The solid residue was washed with methanol (2×100 mL). The combined filtrates were concentrated under reduced pressure. The residue was purified by reverse phase column chromatography (Agela Technologies Claricep™ Flash AQ C18 Column, 20-35 μm, 100 Å, 960 g) eluted with acetonitrile in water from 35% to 45% to give the title compound (5.5 g, 9.20 mmol, 43.6% yield). ¹H NMR (400 MHZ, methanol-d₄) δ ppm 6.91 (s, 1H), 4.52 (dd, J=5.1, 17.0 Hz, 1H), 4.30-4.20 (m, 1H), 4.03-3.94 (m, 1H), 3.88 (dd, J=1.1, 4.2 Hz, 1H), 3.76 (s, 3H), 3.68 (br d, J=4.0 Hz, 1H), 3.00-2.81 (m, 1H), 2.31 (ddd, J=6.7, 11.9, 15.1 Hz, 1H), 1.49 (s, 9H).

### Example 93C: methyl [{(6R,7S)-7-[(tert-butoxycarbonyl)amino]-1-fluoro-3,6-dihydroxy-5,6,7,8-tetrahydronaphthalen-2-yl}(trifluoroacetyl)amino]acetate

To a mixture of Pd—C (5 g, 4.70 mmol) in tetrahydrofuran (200 mL) was added the product of Example 93B (5.5 g, 8.36 mmol) at 25° C. The resulting mixture was stirred at 25° C. under H₂ (15 psi) for 12 hours. Additional Pd—C (5 g, 47.0 mmol) was added into the reaction mixture, the resulting mixture was degassed and purged with H₂ for three times, then stirred under H₂ (15 psi) at 25° C. for another 12 hours. One additional reaction on 0.5 g scale was run as described above. The mixtures were combined and filtered. The filter cake was washed with methanol (3×50 mL), the combined filtrates were concentrated under reduced pressure to give the title compound (5 g, 8.33 mmol, crude used directly).

### Example 93D: methyl [{(6R,7S)-3-(benzyloxy)-7-[(tert-butoxycarbonyl)amino]-1-fluoro-6-hydroxy-5,6,7,8-tetrahydronaphthalen-2-yl}(trifluoroacetyl)amino]acetate

To a mixture of the product of Example 93C (4.5 g, 7.49 mmol, crude) and K₂CO₃ (1.553 g, 11.24 mmol) in N,N-dimethylformamide (200 mL) was added benzyl bromide (1.538 g, 8.99 mmol) at 20° C. The mixture was stirred at 20° C. for 12 hours. One additional reaction on 500 mg scale was run as described above. The reaction mixtures were poured into water (1000 mL) and extracted with ethyl acetate (3×200 mL). The combined organic phases were washed with brine (4×100 mL), dried over Na₂SO₄, filtered, and concentrated under reduced pressure. The residue was purified by column chromatography on silica gel eluting with ethyl acetate in petroleum ether from 20% to 50% to afford the title compound (5.2 g, 7.75 mmol, 93% yield for 2 steps, purity 85%). ¹H NMR (400 MHZ, CDCl₃) δ ppm 7.43-7.32 (m, 6H), 6.57 (s, 1H), 5.06 (br t, J=3.1 Hz, 2H), 4.63 (dd, J=2.9, 16.8 Hz, 1H), 4.31-4.20 (m, 1H), 4.04-3.94 (m, 2H), 3.69-3.65 (m, 3H), 3.15-2.98 (m, 2H), 2.98-2.93 (m, 2H), 2.90 (s, 2H), 2.81-2.68 (m, 1H), 1.51-1.44 (s, 9H).

### Example 93E: methyl [{(7S)-3-(benzyloxy)-7-[(tert-butoxycarbonyl)amino]-1-fluoro-6-oxo-5,6,7,8-tetrahydronaphthalen-2-yl}(trifluoroacetyl)amino]acetate

To a solution of the product of Example 93D (2.5 g, 3.72 mmol, purity 85%) in dichloromethane (50 mL) was added Dess-Martin periodinane (3.16 g, 7.45 mmol) in portions at 0° C. The mixture was stirred at 20° C. for 5 hours. One additional reaction on 2.5 g scale was run as described above. The reaction mixtures were poured into saturated aqueous Na₂SO₃ (50 mL). The mixture was filtered, and the residue cake was washed with dichloromethane (2×50 mL). The resulting biphasic filtrate was separated. The organic phase was washed by brine (40 mL), dried over anhydrous Na₂SO₄, and concentrated under reduced pressure to give the title product (4.5 g, crude) which was used directly in the next step without further purification.

### Example 93F: methyl [{(6S,7S)-3-(benzyloxy)-7-[(tert-butoxycarbonyl)amino]-1-fluoro-6-hydroxy-5,6,7,8-tetrahydronaphthalen-2-yl}(trifluoroacetyl)amino]acetate

To a mixture of the product of Example 93E (4.50 g, 7.92 mmol, crude) in tetrahydrofuran (100 mL) was added NaBH₄ (0.599 g, 15.84 mmol) in portions at 0° C. The mixture was stirred at 0° C. for 0.5 hour. The reaction was quenched with saturated aqueous NH₄Cl (100 mL) at 0° C., and the mixture was extracted with ethyl acetate (3×100 mL). The combined organic layers were washed with brine (40 mL) and concentrated under reduced pressure. The residue was purified by reverse phase column (Agela Technologies Claricep™ Flash AQ C18 Column, 20-35 μm, 100 Å, 330 g, eluted with water in acetonitrile from 40% to 60%) to give the title compound (2.7 g, 3.79 mmol, 47.8% yield, purity 80%). ¹H NMR (400 MHz, CDCl₃) δ ppm 7.44-7.32 (m, 5H), 6.57 (s, 1H), 5.15-5.00 (m, 2H), 4.64 (br dd, J=1.8, 16.9 Hz, 2H), 4.06-3.80 (m, 3H), 3.67 (s, 3H), 3.33-3.10 (m, 2H), 2.96-2.79 (m, 1H), 2.61-2.44 (m, 1H), 1.48 (s, 9H).

### Example 93G: methyl {[(6S,7S)-3-(benzyloxy)-7-[(tert-butoxycarbonyl)amino]-6-{[tert-butyl(dimethyl)silyl]oxy}-1-fluoro-5,6,7,8-tetrahydronaphthalen-2-yl](trifluoroacetyl)amino}acetate

To a mixture of the product of Example 93F (2.7 g, 3.79 mmol, purity 80%) and imidazole (0.387 g, 5.68 mmol) in N,N-dimethylformamide (50 mL) was added tert-butyldimethylchlorosilane (0.685 g, 4.54 mmol) in portions at 20° C. The mixture was stirred at 20° C. for 12 hours before it was diluted with saturated aqueous NH₄Cl (200 mL). The resulting mixture was extracted with ethyl acetate (3×200 mL). The combined organic fractions were washed with brine (5×100 mL), dried over anhydrous Na₂SO₄, and concentrated under reduced pressure. The residue was purified by column chromatography on silica gel eluted with ethyl acetate in petroleum ether from 20% to 30% to give the title compound (2.4 g, 3.15 mmol, 83% yield, purity 90%). ¹H NMR (400 MHZ, CDCl₃) δ ppm 7.44-7.32 (m, 5H), 6.54 (s, 1H), 5.11-4.99 (m, 2H), 4.62 (dd, J=5.7, 16.8 Hz, 1H), 4.52-4.40 (m, 1H), 4.10-3.95 (m, 2H), 3.93-3.82 (m, 1H), 3.67 (s, 3H), 3.22 (br dd, J=6.4, 16.4 Hz, 1H), 3.09-2.96 (m, 1H), 2.77 (td, J=5.4, 17.4 Hz, 1H), 2.58 (br dd, J=4.9, 17.0 Hz, 1H), 1.50-1.41 (s, 9H), 0.91 (s, J=13.3 Hz, 9H), 0.15-0.10 (s, 6H).

### Example 93H: methyl {[(6S,7S)-3-(benzyloxy)-7-[(tert-butoxycarbonyl)amino]-6-{[tert-butyl(dimethyl)silyl]oxy}-1-fluoro-5,6,7,8-tetrahydronaphthalen-2-yl]amino}acetate

To a mixture of the product of Example 93G (1 g, 1.314 mmol, purity 90%) in anhydrous methanol (40 mL) was added sodium methanolate (0.473 g, 2.63 mmol) at 20° C. The mixture was stirred at 60° C. for 3 hours. The mixture was quenched with saturated aqueous NH₄Cl (50 mL) at 0° C. and extracted with ethyl acetate (3×50 mL). The organic layer was washed with brine (40 mL), dried over anhydrous Na₂SO₄, and concentrated under reduced pressure to give the title compound (1 g, crude, used directly). MS (ESI⁺) m/z 533 [M+H—C(CH₃)₃]⁺.

### Example 931; methyl {[(6S,7S)-3-(benzyloxy)-7-[(tert-butoxycarbonyl)amino]-6-{[tert-butyl(dimethyl)silyl]oxy}-1-fluoro-5,6,7,8-tetrahydronaphthalen-2-yl]({[(prop-2-en-1-yl)oxy]carbonyl}sulfamoyl)amino}acetate

To a solution of chlorosulfonyl isocyanate (0.962 g, 6.79 mmol) in dichloromethane (30 mL) was added allyl alcohol (0.395 g, 6.79 mmol) dropwise at 0° C. The mixture was stirred at 0° C. for 30 minutes. To this mixture was added a solution of the product of Example 93H (1 g, 1.698 mmol, crude) and N,N-diisopropylethylamine (1.098 g, 8.49 mmol) in dichloromethane (20 mL) dropwise at 0° C. The mixture was stirred at 0° C. for 30 minutes before it was poured into water (30 mL). The mixture was extracted with dichloromethane (3×40 mL). The combined organic phases were dried over Na₂SO₄ and concentrated under reduced pressure. The residue was purified by flash column on silica gel eluted with ethyl acetate in petroleum ether from 40% to 50% to afford the title compound (0.6 g, 0.876 mmol, 51.6% yield). MS (ESI⁺) m/z 652 [M+H—C(O)OC(CH₃)₃]⁺.

### Example 93J: tert-butyl [(2S,3S)-6-(benzyloxy)-3-{[tert-butyl(dimethyl)silyl]oxy}-8-fluoro-7-(1,1,4-trioxo-1λ⁶,2,5-thiadiazolidin-2-yl)-1,2,3,4-tetrahydronaphthalen-2-yl]carbamate

To a solution of the product of Example 93I (0.8 g, 1.064 mmol) and K₂CO₃ (0.735 g, 5.32 mmol) in methanol (15 mL) was added tetrakis(triphenylphosphine)palladium(0) (0.615 g, 0.532 mmol) under N₂ at 20° C. The reaction mixture was stirred under N₂ at 20° C. for 12 hours before it was poured into saturated NH₄Cl (50 mL). The mixture was extracted with ethyl acetate (3×50 mL). The combined organic fractions were washed with brine (20 mL), dried over Na₂SO₄, and concentrated under reduced pressure. The residue was purified by column chromatography on silica gel eluted with methanol in ethyl acetate from 0% to 10% to afford the tittle compound (300 mg, crude, used directly). MS (ESI⁻) m/z 631 [M−H]⁻.

### Example 93K: 5-[(6S,7S)-7-amino-3-(benzyloxy)-6-{[tert-butyl(dimethyl)silyl]oxy}-1-fluoro-5,6,7,8-tetrahydronaphthalen-2-yl]-1λ⁶,2,5-thiadiazolidine-1,1,3-trione-trifluoroacetate

To a solution of the product of Example 93J (440 mg, 0.415 mmol, crude) in dichloromethane (15 mL) was added trifluoroacetic acid (5 mL, 64.9 mmol) dropwise at 0° C. The mixture was stirred at 0° C. for 30 minutes before it was concentrated under reduced pressure at 30° C. to afford the title compound (175 mg, crude) which was used for next step directly. MS (ESI⁻) m/z 534 [M−H]⁻.

### Example 93L: 5-{(6S,7S)-3-(benzyloxy)-6-{[tert-butyl(dimethyl)silyl]oxy}-1-fluoro-7-[(3-methylbutyl)amino]-5,6,7,8-tetrahydronaphthalen-2-yl}-1λ⁶,2,5-thiadiazolidine-1,1,3-trione

To a solution of the product of Example 93K (175 mg, crude) in dichloromethane (10 mL) and ethanol (10 mL) was added triethylamine (0.182 mL, 1.307 mmol) at 25° C. Then a solution of 3-methylbutanal (84 mg, 0.980 mmol) in dichloromethane (5 mL) was added dropwise at 25° C. The mixture was stirred at 25° C. for 2 hours. Then NaBH₄ (49.4 mg, 1.307 mmol) was added at 0° C. in portions and the resulting mixture was stirred for 20 minutes at 25° C. The reaction mixture was then diluted with methanol (10 mL) and concentrated under reduced pressure. The residue was purified by reverse phase column (Agela Technologies Claricep™ Flash AQ C18 Column, 20-35 μm, 100 Å, 120 g, eluted with acetonitrile in water from 50% to 60%, flow rate 80 mL/minute) to afford the title compound (300 mg, 0.347 mmol, 93% yield, purity 70%), MS (ESI⁻) m/z 604 [M−H]⁻.

### Example 93M: 5-{(6S,7S)-1-fluoro-3,6-dihydroxy-7-[(3-methylbutyl)amino]-5,6,7,8-tetrahydronaphthalen-2-yl}-1λ⁶,2,5-thiadiazolidine-1,1,3-trione

To a solution of the product of Example 93L (275 mg, 0.318 mmol) and HCl (1.589 mL, 1 N aqueous) in tetrahydrofuran (50 mL) was added 20% Pd (OH) 2/C (446 mg, 0.318 mmol) at 20° C. under N₂. The reaction mixture was charged with H₂ three times and stirred under H₂ (15 psi) at 20° C. for 12 hours. The mixture was filtered, and the filtrate was concentrated under reduced pressure to give the phenol intermediate. The phenol intermediate was dissolved in a mixture of acetic acid (9 mL), tetrahydrofuran (3 mL) and H₂O (3 mL) and stirred at 20° C. for 12 hours. The reaction mixture was concentrated under reduced pressure. The residue was purified by preparative HPLC on Phenomenex® Luna® C18 100 mm×30 mm, 5 μm, eluted with acetonitrile-0.075% (v/v) trifluoroacetic acid/H₂O with a gradient 0-30% for 10 minutes and 30-100% for 2 minutes) at a flow rate of 25 mL/minute to afford the title compound (12 mg, 0.026 mmol, 8.28% yield, purity 88%). ¹H NMR (400 MHZ, methanol-d₄) δ ppm 6.55 (s, 1H), 4.29 (s, 2H), 3.98 (td, J=10.07, 5.50 Hz, 1H), 3.35-3.45 (m, 2H), 3.09-3.27 (m, 3H), 2.84 (br dd, J=16.32, 10.32 Hz, 1H), 2.62-2.76 (m, 1H), 1.56-1.82 (m, 3H), 1.02 (dd, J=6.38, 2.00 Hz, 6H); MS (ESI⁻) m/z 402 [M−H]⁻.

### Biological Assays

### Abbreviations

BSA for bovine serum albumin; BID for bis in die (Latin), twice a day; DMEM for Dulbecco's modified Eagle's medium; DMSO for dimethyl sulfoxide; DTT for dithiothreitol; EDTA for ethylenediaminetetraacetic acid; EGTA for ethylene glycol-bis(2-aminoethylether)-N,N,N′,N′-tetraacetic acid; FACS buffer for flow cytometry staining buffer; FBS for fetal bovine serum; HEPES for 4-(2-hydroxyethyl) piperazine-1-ethanesulfonic acid; IFNγ for interferon gamma; MFI for mean fluorescence intensity; PBS for phosphate-buffered saline; PE labeled for phydoerythrin labeled; PEG for polyethylene glycol; RPMI 1640 for Roswell Park Memorial Institute 1640) medium; S-MEM for minimum essential medium Eagle, Spinner modification; TGI for tumor growth inhibition; TNFα for tumor necrosis factor alpha; and Tween® 20 for polyethylene glycol sorbitan monolaurate.

### Example 94: Mobility Shift Assay Used to Determine Potency of PTPN2 Inhibitors

Compound activity was determined using in house His tagged PTPN2 (TC45) protein (SEQ ID NO: 1) in an in vitro enzymatic reaction. The enzymatic assay used to determine activity was a mobility shift assay using a LabChip EZ Reader by Caliper Life Sciences. The enzymatic reaction was carried out in assay buffer (50 mM HEPES pH 7.5, 1 mM EGTA, 10 mM EDTA, 0.01% Tween® 20, and 2 mM DTT). The compounds were dispensed on a white 384 well ProxiPlate™ (PerkinElmer Catalog #6008289) plate using the Labcyte Echo at varying concentrations (12 point, 1:3 dilution). The enzyme (at 0.5 nM) was incubated with compound for 10 minutes at room temperature. Then the substrate (phosphorylated insulin receptor probe sequence: ((OG488)-(NH—CH₂—CH₂—O—CH₂—CH₂—O—CH₂—CO)-T-R-D-I-(PY)-E-T-D-Y—Y—R—K—K—NH₂) (SEQ ID NO: 2) was added at 2 μM to the plates and incubated for another 10 minutes at room temperature. Finally, a quench solution (water and 4-bromo-3-(2-oxo-2-propoxyethoxy)-5-(3-{[1-(phenylmethanesulfonyl)piperidin-4-yl]amino}phenyl)thiophene-2-carboxylic acid) was added to the plates, which were then run on the EZ Reader (excitation 488 nm, emission 530 nm) to measure % conversion (the amount of phosphorylated substrate which was de-phosphorylated by PTPN2). Each plate had a 100% control (inhibitor: 4-bromo-3-(2-oxo-2-propoxyethoxy)-5-(3-{[1-(phenylmethanesulfonyl)piperidin-4-yl]amino}phenyl)thiophene-2-carboxylic acid) and 0) % control (DMSO), which were used to calculate % inhibition. The % inhibition was then used to calculate the IC₅₀ values.

### Example 95: Mobility Shift Assay (MSA) Used to Determine Potency of PTPN1 Inhibitors

Compound activity was determined using in house His tagged full-length PTPN1 protein (SEQ ID NO: 3) in an in vitro enzymatic reaction. The enzymatic assay used to determine activity is a mobility shift assay using a LabChip EZ Reader by Caliper Life Sciences. The enzymatic reaction was carried out in assay buffer (50 mM HEPES pH 7.5, 1 mM EGTA, 10 mM EDTA. 0.01% Tween® 20, and 2 mM DTT). The compounds were dispensed on a white 384 well ProxiPlate™ (PerkinElmer Cat #6008289) plate using a Labcyte Echo® liquid handler at varying concentrations (12 point, 1:3 dilution). The enzyme (at 0.5 nM) was incubated with compound for 10 minutes at room temperature. Then the substrate (phosphorylated insulin receptor probe sequence: ((OG488)-(NH—CH₂—CH₂—O—CH₂—CH₂—O—CH₂—CO)-T-R-D-I-(PY)-E-T-D-Y—Y—R—K—K—NH₂) (SEQ ID NO: 2) was added at 2 μM to the plates and incubated for another 10 minutes at room temperature. Finally, a quench solution (water and 4-bromo-3-(2-oxo-2-propoxyethoxy)-5-(3-{[1-(phenylmethanesulfonyl)piperidin-4-yl]amino}phenyl)thiophene-2-carboxylic acid) was added to the plates, which were then run on the EZ Reader (excitation 488 nm, emission 530 nm) to measure % conversion (the amount of phosphorylated substrate which was de-phosphorylated by PTPN1). Each plate had a 100% control (inhibitor: 4-bromo-3-(2-oxo-2-propoxyethoxy)-5-(3-{[1-(phenylmethanesulfonyl)piperidin-4-yl]amino}phenyl)thiophene-2-carboxylic acid) and 0% control (DMSO), which were used to calculate % inhibition. The % inhibition was then used to calculate the IC₅₀ values.

Table 2 below summarizes the IC₅₀ data obtained using the PTPN2 MSA assay and the PTPN1 MSA assay for exemplary compounds of the disclosure. In this table, “A” represents an IC₅₀ of less than 1 nM; “B” an IC₅₀ of between 1 nM and 10 nM; “C” an IC₅₀ of greater than 10 nM to 100 nM; and “D” an IC₅₀ of greater than 100 nM.

TABLE 2
IC₅₀ values of exemplary compounds of the disclosure
in the PTPN2 and PTPN1 Mobility Shift Assays (MSA).

| | PTPN2 | PTPN1 | | PTPN2 | PTPN1 |
|---|---|---|---|---|---|
| | MSA | MSA | | MSA | MSA |
| Compound | IC₅₀ | IC₅₀ | Compound | IC₅₀ | IC₅₀ |
| No. | (nM) | (nM) | No. | (nM) | (nM) |
| 100 | C | B | 101 | B | B |
| 102 | A | B | 103 | C | |
| 104 | C | C | 105 | B | B |
| 106 | C | C | 107 | C | C |
| 108 | C | C | 109 | C | C |
| 110 | C | C | 111 | C | C |
| 112 | D | D | 113 | D | D |
| 114 | C | C | 115 | C | C |
| 116 | C | C | 117 | C | C |
| 118 | B | B | 119 | B | B |
| 120 | B | B | 121 | B | C |
| 122 | B | C | 123 | B | B |
| 124 | B | B | 125 | B | C |
| 126 | B | B | 127 | B | B |
| 128 | B | B | 129 | B | B |
| 130 | B | B | 131 | B | B |
| 132 | C | C | 133 | B | B |
| 134 | C | C | 135 | C | C |
| 136 | C | C | 137 | A | B |
| 138 | C | C | 139 | B | C |
| 140 | C | C | 141 | B | C |
| 142 | B | C | 143 | C | D |
| 144 | B | C | 145 | C | C |
| 146 | B | | 147 | C | C |
| 148 | A | A | 149 | B | C |
| 150 | B | B | 151 | B | C |
| 152 | B | | 153 | C | C |
| 154 | B | C | 155 | C | C |
| 156 | B | B | 157 | B | C |
| 158 | C | D | 159 | C | C |
| 160 | C | C | 161 | B | C |
| 162 | B | C | 163 | C | D |
| 164 | C | D | 165 | C | C |
| 166 | A | B | 167 | B | C |
| 168 | C | C | 169 | C | |
| 170 | C | D | 171 | B | B |
| 172 | C | C | 173 | B | B |
| 174 | B | B | 175 | B | C |
| 176 | C | C | 177 | A | B |
| 178 | A | B | 179 | C | C |
| 180 | C | D | 181 | B | B |
| 182 | C | C | 183 | D | D |
| 184 | D | D | 185 | B | B |
| 186 | C | C | 187 | B | B |
| 188 | C | C | 189 | B | D |
| 190 | B | C | 191 | C | C |
| 192 | C | C | 193 | D | C |
| 194 | B | B | 195 | C | C |
| 196 | C | C | 197 | C | C |
| 198 | C | D | 199 | A | A |
| 200 | A | A | 201 | A | A |
| 202 | A | B | 203 | A | B |
| 204 | A | B | 205 | B | C |
| 206 | C | C | 207 | C | C |
| 208 | C | C | 209 | B | B |
| 210 | D | C | 211 | B | B |
| 212 | D | D | 213 | D | D |
| 214 | C | C | 215 | D | D |
| 216 | D | D | 217 | D | D |
| 218 | D | C | 219 | C | C |
| 220 | C | C | 221 | C | B |
| 222 | C | C | 223 | C | B |
| 224 | D | C | 225 | B | C |
| 226 | C | C | 227 | D | D |
| 228 | D | C | 229 | C | C |
| 230 | D | C | 231 | C | B |
| 232 | D | C | 233 | D | C |
| 234 | C | B | 235 | C | C |
| 236 | D | C | 237 | D | D |
| 238 | B | B | 239 | B | B |
| 240 | C | D | 241 | C | C |
| 242 | C | C | 243 | B | B |
| 244 | C | C | 245 | C | C |
| 246 | C | C | 247 | C | C |
| 248 | C | C | 249 | C | C |
| 250 | C | C | 251 | C | C |
| 252 | D | D | 253 | B | B |
| 254 | C | C | 255 | D | C |
| 256 | B | B | 257 | B | B |
| 258 | B | B | 259 | C | C |
| 260 | B | B | 261 | C | C |
| 262 | C | B | 263 | C | |
| 264 | C | C | 265 | C | |
| 266 | B | B | 267 | C | |
| 268 | C | C | 269 | B | B |
| 270 | C | C | 271 | C | C |
| 272 | C | C | 273 | B | B |
| 274 | B | B | 275 | C | C |
| 276 | A | B | 277 | B | C |
| 278 | C | C | 279 | C | C |
| 280 | D | D | 281 | C | C |
| 282 | C | C | 283 | C | D |
| 284 | C | C | 285 | C | |
| 286 | B | B | 287 | C | D |
| 288 | C | B | 289 | C | D |
| 290 | B | B | 291 | B | B |
| 292 | C | C | 293 | C | C |
| 294 | C | C | 295 | C | D |
| 296 | C | D | 297 | A | B |
| 298 | C | C | 299 | B | C |
| 300 | B | B | 301 | C | D |
| 302 | B | C | 303 | B | C |
| 304 | D | D | 305 | C | C |
| 306 | D | D | 307 | B | B |
| 308 | B | B | 309 | C | C |
| 310 | C | C | 311 | B | B |
| 312 | C | C | 313 | B | C |
| 314 | C | D | 315 | B | C |
| 316 | C | D | 317 | C | D |
| 318 | B | C | 319 | C | D |
| 320 | D | D | 321 | B | B |
| 322 | C | C | 323 | B | B |
| 324 | B | C | 325 | B | B |
| 326 | C | C | 327 | A | B |
| 328 | C | C | 329 | C | C |
| 330 | D | D | 331 | B | B |
| 332 | C | C | 333 | C | C |
| 334 | B | B | 335 | C | C |
| 336 | B | B | 337 | C | C |
| 338 | A | B | 339 | C | C |
| 340 | A | B | 341 | C | C |
| 344 | B | C | 345 | C | C |
| 346 | B | C | 347 | C | C |
| 348 | B | C | 349 | C | C |
| 350 | B | C | 351 | C | C |
| 352 | B | B | 353 | B | C |
| 354 | B | C | 355 | B | C |
| 356 | A | B | 357 | C | C |
| 358 | C | C | 359 | C | C |
| 360 | B | C | 361 | B | C |
| 362 | C | C | 363 | B | C |
| 364 | C | C | 365 | B | C |
| 366 | C | C | 367 | C | C |
| 368 | C | C | 369 | B | C |
| 370 | C | D | 371 | A | B |
| 372 | C | C | 373 | B | C |
| 374 | B | C | 375 | C | C |
| 376 | A | B | 377 | A | B |
| 378 | C | | 379 | C | C |
| 380 | C | C | 381 | C | D |
| 382 | C | C | 383 | C | C |
| 384 | C | C | 385 | B | C |
| 386 | C | C | 387 | D | D |
| 388 | D | D | 389 | C | C |
| 390 | C | C | 391 | C | C |
| 392 | C | C | 393 | B | B |
| 394 | C | C | 395 | D | D |
| 396 | C | C | 397 | D | D |
| 398 | C | C | 399 | C | C |
| 400 | C | C | 401 | D | D |
| 402 | D | D | 403 | C | C |
| 404 | C | C | 405 | C | C |
| 406 | C | D | 407 | C | C |
| 408 | B | B | 409 | B | B |
| 410 | B | B | 411 | A | B |

### Example 96: B16F10 IFNγ-Induced Cellular Growth Inhibition Assay

B16F10 mouse melanoma cells (ATCC Cat #CRL-6475, Manassas, VA) were seeded at a density of 500 cells per well in a 384-well clear bottom plate (Corning Cat #3765, Corning, NY) in 25 μL total volume of DMEM+10% FBS (Sigma Cat #D6429 and Sigma Cat #F4135, St. Louis, MO). Cells were allowed to adhere overnight at 37° C.+5% CO₂. On the following day, 12.5 μL of mouse IFNγ (RD systems Cat #485-MI/CF, Minneapolis, MN) was added to half of the plate (columns 13-24) at a concentration of 2 ng/ml for a final assay concentration of 0.5 ng/ml of IFNγ. Media only (12.5 μL of DMEM+10% FBS) was added to the remainder of the plate (columns 1-12). Next, compounds resuspended in DMSO (Sigma Cat #D2650) at 100 mM were diluted in semi-log dilutions in DMSO ranging from 100 mM to 0.001 mM and DMSO only controls were included. The compound/DMSO dilutions were further diluted 1:250 in DMEM+10% FBS, and 12.5 μL of these dilutions were added in triplicates to cells of both treatment arms (with and without IFNγ). Final compound concentrations ranged from 100 μM to 0.001 μM with a final DMSO concentration of 0.1%. Compounds were only dosed in the inner 240 wells, avoiding the outer 2-well perimeter of the plate to minimize edge effects. Finally, the plate was loaded into an IncuCyte® S3 Live Cell Analysis System (Essen Bioscience-Sartorius, Ann Arbor, MI) maintained in a 37° C.+5% CO₂ incubator, allowed to equilibrate for 2 hours, and imaged every 6 hours for 5 days. Confluence over time for compound dilutions in the presence and absence of IFNγ was measured. Growth inhibition values were obtained when the “DMSO/no IFNγ” control reached confluence >95%. At these time points, for each compound the percent growth inhibition at every compound dose level was calculated relative to the “DMSO/with IFNγ” control and used to determine the IC₅₀.

Finding novel strategies to inhibit tumor growth is an active field of research in oncology drug discovery. The growth of certain cancer types, among them melanoma, can be suppressed by IFNγ, a cytokine produced by cells of the immune system like T cells or NK cells. Ablation of IFNγ signaling promotes tumor growth. In contrast, enhancing IFNγ signaling amplifies tumor growth inhibition. Thus, a potent compound should promote tumor growth arrest in the presence of IFNγ.

Compounds of the present disclosure amplify B16F10 melanoma growth inhibition in the presence of IFNγ. Importantly, no tumor growth inhibition is observed in the absence of IFNγ indicating an on target mechanism of the compounds.

Table 3 below summarizes the B16F10 IFNγ-induced cellular growth inhibition IC₅₀ values for exemplary compounds of the disclosure. In this table, “A” represents an IC₅₀ of less than 1 μM: “B” an IC₅₀ of between 1 μM and 10 μM; “C” an IC₅₀ of greater than 10 μM to 100 μM; and “D” an IC₅₀ of greater than 100 μM.

TABLE 3
IC₅₀ values of exemplary compounds of the disclosure in the
B16F10 IFNγ-induced cellular growth inhibition (GI) assay.

| Compound | B16F10 | Compound | B16F10 |
|---|---|---|---|
| No. | GI IC₅₀ (uM) | No. | GI IC₅₀ (μM) |
| 102 | A | 103 | B |
| 104 | C | 106 | C |
| 107 | C | 108 | C |
| 109 | C | 111 | C |
| 112 | C | 116 | B |
| 117 | B | 118 | A |
| 119 | B | 120 | A |
| 122 | B | 123 | B |
| 124 | A | 125 | C |
| 126 | B | 127 | A |
| 128 | B | 129 | A |
| 130 | B | 131 | A |
| 133 | A | 134 | B |
| 135 | B | 136 | D |
| 137 | C | 139 | C |
| 142 | C | 143 | B |
| 145 | B | 146 | A |
| 148 | A | 150 | B |
| 151 | B | 153 | D |
| 155 | C | 156 | A |
| 157 | B | 161 | C |
| 166 | A | 169 | C |
| 172 | B | 173 | A |
| 174 | A | 175 | A |
| 177 | A | 178 | A |
| 179 | B | 180 | B |
| 184 | C | 185 | A |
| 187 | A | 188 | A |
| 189 | B | 190 | B |
| 194 | B | 196 | B |
| 197 | B | 199 | A |
| 200 | A | 202 | A |
| 203 | A | 205 | B |
| 206 | B | 208 | C |
| 209 | B | 211 | A |
| 214 | B | 217 | B |
| 218 | C | 221 | B |
| 223 | A | 225 | B |
| 229 | B | 231 | B |
| 234 | B | 235 | B |
| 238 | B | 241 | C |
| 243 | B | 245 | B |
| 247 | C | 250 | D |
| 251 | B | 252 | C |
| 253 | B | 254 | B |
| 255 | B | 257 | B |
| 258 | B | 259 | B |
| 260 | A | 262 | C |
| 268 | B | 269 | C |
| 276 | B | 286 | B |
| 288 | B | 291 | A |
| 292 | B | 293 | B |
| 306 | D | 307 | A |
| 308 | A | | |

### Example 97: Human Whole Blood pSTAT1 Proximal Pharmacodynamic (PD) Assay

Human blood samples were acquired through internal AbbVie Inc's blood donation program in accordance with AbbVie's Occupational Safety and Health Administration protocols. Blood was collected by venipuncture into sodium heparin coated vacutainer tubes and kept at room temperature for no longer than 1 hour prior to the experiment initiation. Human blood samples (90 μL) were added to the individual wells of 96 well plate containing 10 μL of 10× working stock solutions of increasing concentrations of compounds ranging from 0.025 μM to 500 μM and incubated for 3 hours at 37° C. To induce STAT1 phosphorylation the samples were then treated with recombinant human IFNγ (R&D Systems, Catalog #285-IF, Minneapolis, MN; 100 nM final concentration) for 20 minutes, and 3 μL/well BV421 labeled anti-CD14 surface antibody (Biolegend, San Diego, CA, Catalog #301830) was added for 45 minutes before fixation and red blood cell lysis was performed with BD Phosflow Lyse/Fix buffer (BD Biosciences, San Jose, CA, Catalog #558049). Cells were subsequently permeabilized on ice by the addition of BD Perm III buffer (BD Biosciences, San Jose, CA, Catalog #558050) and stored at −80° C. until use. Before staining, the cells were washed with PBS containing 0.1% BSA. Optimized concentrations of BUV395 labeled anti-CD45 (BD Biosciences, San Jose, CA, Catalog #563792) and PE labeled anti-phospho-STAT1 (pY701: Invitrogen, Carlsbad, CA, Catalog #12-9008-42) antibodies were added to the cell suspensions and incubated for 2 hours. The cells were washed with PBS containing 0.1% BSA and analyzed on a BD LSRFortessa™ X20 flow cytometer (BD Biosciences, San Jose, CA) using BD FACSDiva™ software. The data was analyzed using FlowJo V10 analysis software (Flow Jo LLC, Ashland, OR). The amount of STAT phosphorylation was measured by the mean fluorescence intensity (MFI) of pSTAT1 in CD14+ monocytes. Compound dose-response curves were determined using a four-parameter logistic-nonlinear regression model from which half maximal effective concentrations (EC₅₀) were calculated. All statistical analyses utilized GraphPad software (San Diego, CA).

Protein tyrosine phosphatases PTPN2 and PTPN1 are negative regulators of several cellular pathways among them JAK/STAT mediated cytokine signaling (e.g. IFNγ, IFNα, IL2). Inhibition of PTPN2/N1 is expected to elevate STAT phosphorylation by delaying the dephosphorylation of STAT proteins. The impact of compounds on IFNγ signaling was evaluated via measuring the phosphorylation of the direct PTPN2/N1 target, STAT1, as proximal translational pharmacodynamic markers in human whole blood. The cells contained in whole blood provide a close physiologically relevant setting as well as facilitate assessment of small molecule protein binding characteristics and the amount of free drug available for action on its target. In human whole blood spiked with active compounds, a dose dependent enhancement of STAT1 phosphorylation after stimulation with IFNγ was observed. Compounds of the present disclosure amplify the IFNγ-induced phosphorylation of STAT1. Table 4 below summarizes the pSTAT EC₅₀ values for exemplary compounds of the disclosure.

TABLE 4
Comparison of IC₅₀ values of select compounds in the B16F10 growth
inhibition assay and EC₅₀ values in human whole blood IFNγ-induced
STAT1 phosphorylation.

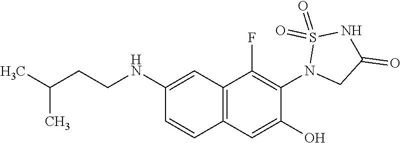 [SMILES: CC(C)CCNc1ccc2cc(O)c(N3CC(=O)NS3(=O)=O)c(F)c2c1]

Compound V

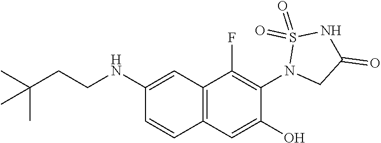 [SMILES: CC(C)(C)CCNc1ccc2cc(O)c(N3CC(=O)NS3(=O)=O)c(F)c2c1]

Compound W

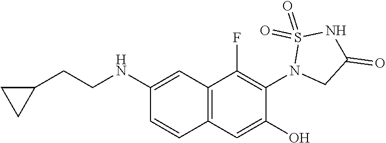 [SMILES: O=C1CN(c2c(O)cc3ccc(NCCC4CC4)cc3c2F)S(=O)(=O)N1]

Compound X

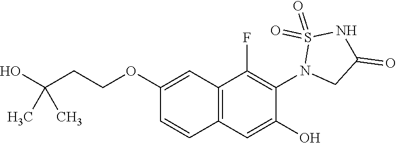 [SMILES: CC(C)(O)CCOc1ccc2cc(O)c(N3CC(=O)NS3(=O)=O)c(F)c2c1]

Compound Y

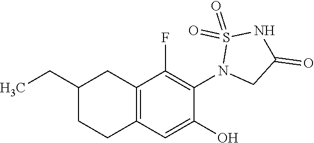 [SMILES: CCC1CCc2cc(O)c(N3CC(=O)NS3(=O)=O)c(F)c2C1]

Compound Z

| | B16F10 Growth | Human Whole Blood IFNγ |
|---|---|---|
| | Inhibition | induced pSTAT1 |
| Compound | IC₅₀ (μM) | EC₅₀ (μM) |
| Compound V | 3.2 | |
| Compound W | 4.1 | |
| Compound X | 4.1 | |
| Compound Y | 5.8 | 38 |
| Compound Z | 17.8 | >500 |
| 118 | 0.11 | 0.58 |
| 133 | 0.22 | 1.6 |
| 146 | <0.05 | 0.4 |
| 177 | 0.14 | 2 |
| 199 | <0.05 | 0.83 |
| 200 | <0.05 | 2.4 |
| 259 | 1.5 | 14.2 |
| 260 | 0.18 | 4.4 |

### Example 98: T Cell Function Assays

Pan T cells were isolated from C57BL6 splenocytes using a MACS Pan T cell isolation kit II (Miltenyi Biotec, Auburn, CA) according to the manufacturer's instructions. Isolated T cells (200,000 cells/well in a 96 well flat-bottom plate) were cultured in RPMI 1640 supplemented with 10% FBS, 50 nM 2-mercatoethanol, 100 U/mL penicillin, and 100 μg/mL streptomycin, and incubated with 0.3 μM compound or DMSO in duplicates. After 1 hour, mouse T cell activator CD3/CD28 Dynabeads (ThermoFisher Scientific, Waltham, MA) were added at a 1:5 beads to cells ratio to stimulate the T cells for 3 days. T cells with or without compound were incubated in the absence of T cell activator beads to evaluate if compounds nonspecifically stimulate the T cells. After 3 days of stimulation, supernatants were collected and IFNγ and TNFα in superatants were assessed using an MSD V-plex assay (Meso Scale Discovery, Rockville, MD).

The increase of T cell activation and most importantly T cell function is a main strategy for novel immune oncology approaches to promote tumor immunity. In vitro assays using primary T cells are commonly used to assess the impact of compound on T cell activation and function.

A read out for T cell function important for tumor immunity is the production of pro-inflammatory, anti-tumorigenic cytokines like IFNγ and TNFα. This can be assessed through the detection of cytokines in the supernatants of in vitro stimulated T cells. An immune stimulatory compound is expected to increase the production of IFNγ and TNFα. Compounds of the present disclosure promote IFNγ and TNFα production of stimulated T cells. Importantly, compounds did not nonspecifically increase IFNγ and TNFA production in the absence of TCR stimulation. Table 5 below summarizes the amount of IFNγ and TNFA produced from T cells either stimulated through the TCR (anti-CD3/CD28) or left unstimulated (no stimulation) for 3 days for exemplary compounds of the disclosure.

TABLE 5
Cytokine data from the T cell function assays.

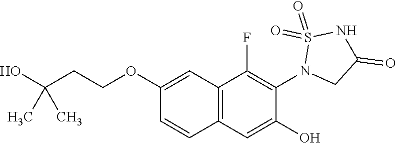 [SMILES: CC(C)(O)CCOc1ccc2cc(O)c(N3CC(=O)NS3(=O)=O)c(F)c2c1]

Compound Y

| | IFNγ [pg/mL] | | TNFα[pg/mL] | |
|---|---|---|---|---|
| | no | anti-CD3/ | no | anti-CD3/ |
| Compound No. | stimulation | CD28 | stimulation | CD28 |
| DMSO | 1.1 | 49 | 1.1 | 73.7 |
| 118 | 1.8 | 261.3 | 1.1 | 159.1 |
| 133 | 1.0 | 204.1 | 0.8 | 142.0 |
| 148 | 1.0 | 168.4 | 0.8 | 123.4 |
| 177 | 1.0 | 168.2 | 0.7 | 113.7 |
| 199 | 1.8 | 212.9 | 1.1 | 147.8 |
| 204 | 1.8 | 148.8 | 1.1 | 105.4 |
| 254 | 0.7 | 97.0 | 1.1 | 91.2 |
| 260 | 0.8 | 139.6 | 1.1 | 100.0 |
| 291 | 0.7 | 83.3 | 1.1 | 91.2 |
| 307 | 1.2 | 66.7 | 1.1 | 89.5 |
| Compound Y | 1.2 | 67.2 | 1.0 | 32.5 |

### Example 99. In Vivo Efficacy of PTPN2 Inhibitors in MC38 Mouse Tumor Model and Impact on Pharmacodynamic Markers

### Mice.

All experiments were conducted in compliance with AbbVie's Institutional Animal Care and Use Committee and the National Institutes of Health Guide for Care and Use of Laboratory Animals guidelines in a facility accredited by the Association for the Assessment and Accreditation of Laboratory Animal Care. C57Bl/6 female mice were obtained from Charles River (Wilmington, MA). The mice were group-housed 10 per cage. Food and water were available ad libitum. Animals were acclimated to the animal facilities for a period of at least one week prior to commencement of experiments. Animals were tested in the light phase of a 12-hour light:12-hour dark schedule (lights on 0600 hours).

### Tumor Cell Inoculation and Treatments.

Cells were grown to passage 3 in vitro. A total of 1×10⁵ viable MC-38 cells were inoculated subcutaneously into the right flank of female C57Bl/6 mice (7-12 weeks old) on Day 0. The injection volume was 0.1 mL and was composed of a 1:1 mixture of S-MEM and Matrigel® (Corning, NY, USA). Tumors were size matched on Day 14 and the mice had a mean body weight of ˜21 g. The mean tumor volume (TV) at size match was approximately 196±64 mm³. Following size match, treatments were initiated on the same day. Dosing of mice was conducted orally, twice a day (BID) at 7 a.m. and 5 p.m. for 21 days. Mice were dosed (10 mg/kg/dose) with either Compound 118 or vehicle controls (n=15 mice/group). Compound 118 was formulated in 10% ethanol, 30% PEG-400 and 60% Phosal-50PG and was dosed at 10 mL/kg. Tumor volume was calculated three times weekly. Measurements of the length (L) and width (W) of the tumor were taken via electronic caliper and the volume was calculated according to the following equation: V=L×W²/2 using Study Director Version 3.1.399.22 (Studylog Systems, Inc, CA, USA). Mice were euthanized when tumor volume was ≤3000 mm³ or skin ulcerations occurred. Tumor growth inhibition (TGI) was calculated as TGI=1−(Mean TV_{Timepoint(Treatment)}/Mean TV_{Timepoint(Vehicle)}) for each timepoint that tumor volumes were measured. Reported TGI_{Max} is the largest TGI value for any timepoint that tumors volumes were collected for that treatment group.

pSTAT5 Flow Cytometry Assay in Mouse Whole Blood.

Whole blood was drawn into EDTA powder coated tubes by cardiac puncture from mice on day 8 of dosing with Compound 118 (2 hours after the 16th dose). 90 μL of whole blood were stimulated with 10 μL of murine IL-2 to achieve a final concentration of 100 ng IL-2/mL (R&D Systems. Minneapolis, MN, cat #402-ML) for 20 minutes at 37° C. 5% CO₂. After stimulation. 1.8 mL of prewarmed BD Phosflow Lyse/Fix Buffer (BD Biosciences. San Jose. CA) was added for 20 minutes at 37° C. Cells were washed twice in FACS buffer (Dulbecco's PBS with 0.2% BSA) and incubated for 30 minutes on ice in cold Perm Buffer III (BD Biosciences. San Jose. CA). Cells were washed with FACS buffer and resuspended in 50 μL of FACS buffer with antibodies and stained for 3 hours at room temperature with gentle shaking. The antibodies added were a combination of the following; anti-CD3-AF647, clone 145-2C11 (Biolegend. Cat #564279); anti-CD4-FITC, clone GK1.5 (Biolegend. San Diego, CA. Cat #100406); anti-pSTAT5 (pY694)-PE, clone 47 (BD Biosciences. San Jose, CA. Cat #562077); anti-CD45-BUV395, clone 30-F11 (BD Biosciences. San Jose, CA, cat #564279). After staining, cells were washed twice with FACS buffer, and the samples were acquired on a BD LSRFortessa™ X20 flow cytometers (BD Biosciences. San Jose, CA) and analyzed with FLowJo V10 software (FlowJo, Ashland, OR). The mean fluorescence intensity (MFI) of pSTAT5 as a measure of the amount of phosphorylated STAT5 in the CD3+ T cell population from vehicle or Compound 118 treated animals was reported.

### Granzyme B Staining of CD8 T Cells Flow Cytometry Assay in Mouse Spleen.

Mice were sacrificed on day 8 of dosing with Compound 118 (2 hours after the 16^{th} dose) and spleens were excised. Spleens were dissociated with a gentleMACS dissociator (Miltenyi Biotec. Bergisch Gladbach. Germany), red blood cells lysed, and single cell suspensions were prepared. Splenocytes were stained with Zombie UV™ Fixable Viability kit (Biolegend. San Diego, CA) diluted in Dulbecco's PBS for 10 minutes at room temperature to exclude dead cells followed by staining for surface markers for 45 minutes on ice using the following flow cytometry antibodies diluted in autoMACS Running Buffer (Miltenyi Biotec. Bergisch Gladbach, Germany); Brilliant Violet 510)-labeled anti-CD45. Brilliant Ultraviolet 395-labeled anti-CD3. Brilliant Violet 786-labeled anti-CD4. APC/Cy7-labeled anti-CD8. Cells were washed twice with autoMACS Running Buffer, permeabilized with Fixation/Permeabilization buffer (FoxP3/Transcription Factor Staining Buffer Set; eBioscience) and stained intracellularly with PE-labeled anti-Granzyme B antibody diluted in Permeabilization buffer (FoxP3/Transcription Factor Staining Buffer Set; eBioscience, San Diego, CA) for 1 hour on ice. After staining, cells were washed twice with autoMACS Running Buffer, and the samples were acquired on a BD LSRFortessa™ X20 flow cytometers (BD Biosciences, San Jose, CA) and analyzed with FLowJo V10 software (FlowJo, Ashland, OR). The frequency of Granzyme B+ cells within the CD8+ T cell population in vehicle or Compound 118 treated animals was reported.

### Cytokine Measurement in Mouse Plasma.

Whole blood was drawn into EDTA powder coated tubes by cardiac puncture from mice on day 8 of dosing with Compound 118 (2 hours after the 16^{th} dose) and plasma was prepared by centrifugation. Cytokines in plasma were measured using the Th1/Th2 Cytokine & Chemokine 20-Plex Mouse ProcartaPlex™ Panel 1 (Invitrogen, Carlsbad, CA). IP10 levels (pg/mL) in vehicle or Compound 118 treated animals were reported.

### Results

Expression within tumor cells of the phosphatases PTPN2 and its highly homologous counterpart, PTPN1, were recently described to be negative regulators of tumor-directed immune responses. The functional activity of PTPN2 to inhibit signaling cascades of extrinsic factors within tumor cells, particularly de-phosphorylation of STAT molecules downstream of the IFNγ receptor was defined as a significant contributor to the ability of tumor cells to evade or suppress anti-tumor immune responses. To confirm these claims, specific inhibitors of PTPN2/1B were created and tested for their ability to inhibit tumor growth and elicit anti-tumor inflammation in an in vivo syngeneic mouse tumor model. Mice were inoculated on their hind flank with the murine colon adenocarcinoma, MC-38. Following two weeks of tumor cell growth, mice began oral BID treatment for 21 days with either the vehicle or the formulated Compound 118. Compound 118 was well tolerated, without obvious adverse health events. However, within 7-10 days of treatment, apparent tumor stasis and shrinkage was observed in animals dosed with Compound 118. Eventually, 70% of mice treated with Compound 118 achieved complete cures, and an overall TGI_{Max} of 94% (Table 6). Significant tumor efficacy observed with Compound 118 was followed by further examination of direct target engagement of the compounds in vivo as well as their effects on anti-tumor immune responses.

IL2 signaling in T cells promotes T cell homeostasis and proliferation. STAT5 is a signaling molecule in the IL2 pathway and a direct target of PTPN2 and PTPN1 which serve as negative regulators of IL2 signaling. A PTPN2/1B inhibitor is expected to increase the phosphorylation of STAT5 upon stimulation with IL2. To demonstrate in vivo target engagement, we measured pSTAT5 levels in T cells from whole blood of PTPN2/1B inhibitor dosed animals after in vitro stimulation of whole blood with IL2. In mice treated with Compound 118, pSTAT5 levels in whole blood T cells was 1.6-fold higher (MFI=1261±97) than in vehicle control treated animals (MFI=802±52) (Table 6).

One desirable effect of immunotherapy is the induction of functional cytotoxic T cells which can improve tumor immunity. In Compound 118 treated mice, the frequency of functional, granzyme B (GzB) producing cells within the cytotoxic CD8±T population in the spleen was 3.9-fold higher (4.3±0.9%) than in vehicle control treated animals (1.1±0.1%) (Table 6).

Because a PTPN2/1B inhibitor promotes IFNγ signaling by increasing the phosphorylation of JAK and STAT signaling molecules and IP10 is an IFNγ induced protein, a PTPN2/1B inhibitor is expected to increase the production of IP10. IP10 levels in plasma of Compound 118 treated mice were 1.7-fold higher (256±30 μg/mL) than in vehicle control treated animals (153±15 μg/mL) (Table 6).

TABLE 6
Impact of oral BID dosing with indicated treatment on tumor
growth and PD marker movement in the MC-38 syngeneic tumor model.
TGI_{Max} was determined over the entirety of the study. PD markers
were evaluated on day 8 of dosing (2 hours post 16^{th} dose).
Data are represented as value ± SEM.

| | Tumor | | pSTAT5 level | |
|---|---|---|---|---|
| | Growth | | [MFI] in | |
| | Inhibition | | CD3 + T cells | |
| | (Max) | % GzB + cells | from IL2 | IP10 in |
| | compared to | within splenic | stimulated | plasma |
| Compound | vehicle [%] | CD8 + T cells | whole blood | [pg/mL] |
| Vehicle | — | 1.1 ± 0.1 | 802 ± 52 | 153 ± 15 |
| 118 | 94 | 4.3 ± 0.9 | 1261 ± 97 | 256 ± 30 |

### EQUIVALENTS AND SCOPE

In the claims, articles such as “a.” “an.” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-63. (canceled)

64. A compound selected from the group consisting of

| Com- | |
|---|---|
| pound | |
| Num- | Structure |
| ber | |
| 326 | 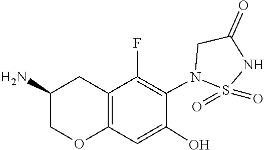 [SMILES: N[C@@H]1COc2cc(O)c(N3CC(=O)NS3(=O)=O)c(F)c2C1] |
| 327 | 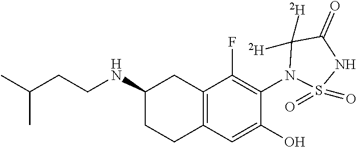 [SMILES: [2H]C1([2H])C(=O)NS(=O)(=O)N1c1c(O)cc2c(c1F)C[C@H](NCCC(C)C)CC2] |
| 328 | 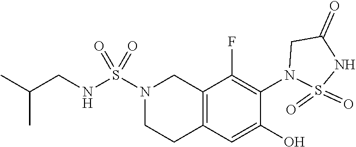 [SMILES: CC(C)CNS(=O)(=O)N1CCc2cc(O)c(N3CC(=O)NS3(=O)=O)c(F)c2C1] |
| 329 | 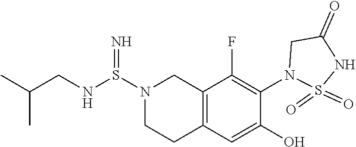 [SMILES: CC(C)CNS(=N)N1CCc2cc(O)c(N3CC(=O)NS3(=O)=O)c(F)c2C1] |
| 330 | 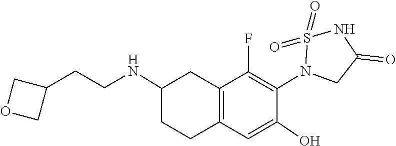 [SMILES: O=C1CN(c2c(O)cc3c(c2F)CC(NCCC2COC2)CC3)S(=O)(=O)N1] |
| 331 | 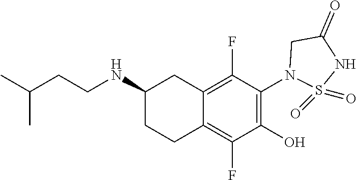 [SMILES: CC(C)CCN[C@@H]1CCc2c(F)c(O)c(N3CC(=O)NS3(=O)=O)c(F)c2C1] |
| 332 | 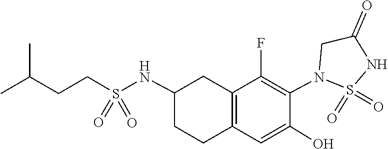 [SMILES: CC(C)CCS(=O)(=O)NC1CCc2cc(O)c(N3CC(=O)NS3(=O)=O)c(F)c2C1] |
| 333 | 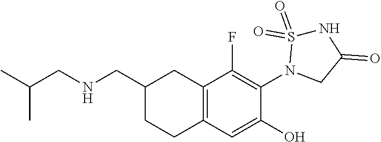 [SMILES: CC(C)CNCC1CCc2cc(O)c(N3CC(=O)NS3(=O)=O)c(F)c2C1] |
| 334 | 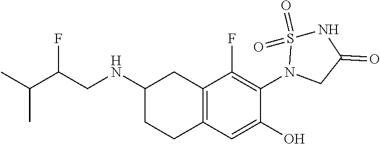 [SMILES: CC(C)C(F)CNC1CCc2cc(O)c(N3CC(=O)NS3(=O)=O)c(F)c2C1] |
| 335 | 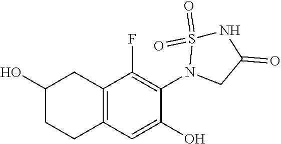 [SMILES: O=C1CN(c2c(O)cc3c(c2F)CC(O)CC3)S(=O)(=O)N1] |
| 336 | 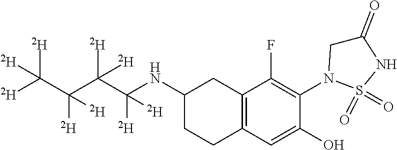 [SMILES: [2H]C([2H])([2H])C([2H])([2H])C([2H])([2H])C([2H])([2H])NC1CCc2cc(O)c(N3CC(=O)NS3(=O)=O)c(F)c2C1] |
| 337 | 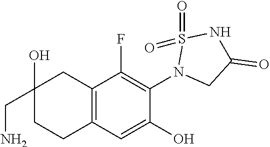 [SMILES: NCC1(O)CCc2cc(O)c(N3CC(=O)NS3(=O)=O)c(F)c2C1] |
| 338 | 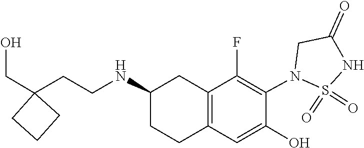 [SMILES: O=C1CN(c2c(O)cc3c(c2F)C[C@H](NCCC2(CO)CCC2)CC3)S(=O)(=O)N1] |
| 339 | 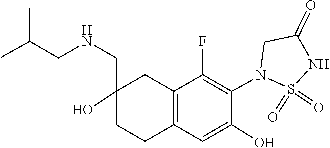 [SMILES: CC(C)CNCC1(O)CCc2cc(O)c(N3CC(=O)NS3(=O)=O)c(F)c2C1] |
| 340 | 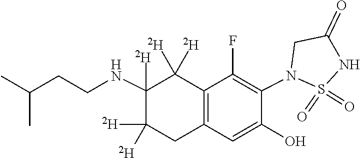 [SMILES: [2H]C1([2H])Cc2cc(O)c(N3CC(=O)NS3(=O)=O)c(F)c2C([2H])([2H])C1([2H])NCCC(C)C] |
| 341 | 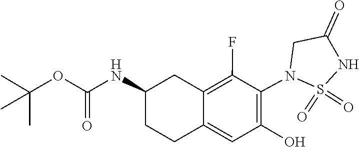 [SMILES: CC(C)(C)OC(=O)N[C@@H]1CCc2cc(O)c(N3CC(=O)NS3(=O)=O)c(F)c2C1] |
| 342 | 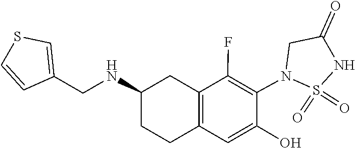 [SMILES: O=C1CN(c2c(O)cc3c(c2F)C[C@H](NCc2ccsc2)CC3)S(=O)(=O)N1] |
| 343 | 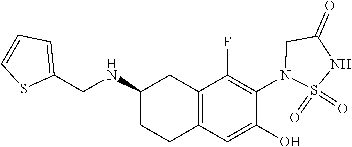 [SMILES: O=C1CN(c2c(O)cc3c(c2F)C[C@H](NCc2cccs2)CC3)S(=O)(=O)N1] |
| 344 | 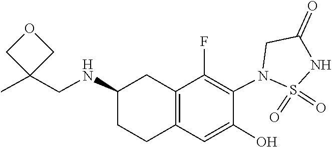 [SMILES: CC1(CN[C@@H]2CCc3cc(O)c(N4CC(=O)NS4(=O)=O)c(F)c3C2)COC1] |
| 345 | 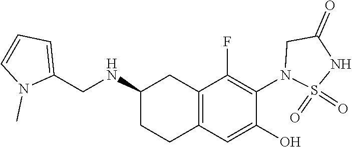 [SMILES: Cn1cccc1CN[C@@H]1CCc2cc(O)c(N3CC(=O)NS3(=O)=O)c(F)c2C1] |
| 346 | 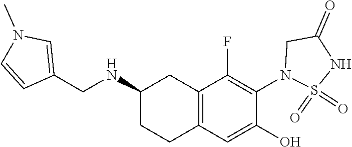 [SMILES: Cn1ccc(CN[C@@H]2CCc3cc(O)c(N4CC(=O)NS4(=O)=O)c(F)c3C2)c1] |
| 347 | 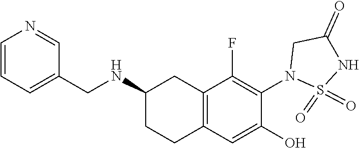 [SMILES: O=C1CN(c2c(O)cc3c(c2F)C[C@H](NCc2cccnc2)CC3)S(=O)(=O)N1] |
| 348 | 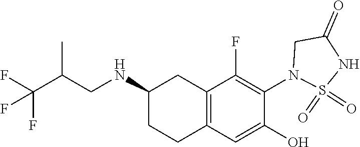 [SMILES: CC(CN[C@@H]1CCc2cc(O)c(N3CC(=O)NS3(=O)=O)c(F)c2C1)C(F)(F)F] |
| 349 | 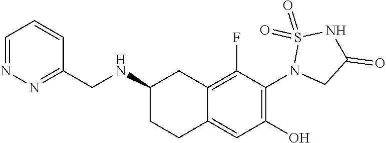 [SMILES: O=C1CN(c2c(O)cc3c(c2F)C[C@H](NCc2cccnn2)CC3)S(=O)(=O)N1] |
| 350 | 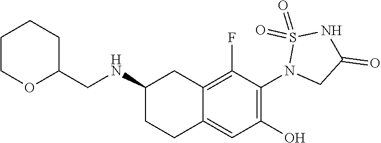 [SMILES: O=C1CN(c2c(O)cc3c(c2F)C[C@H](NCC2CCCCO2)CC3)S(=O)(=O)N1] |
| 351 | 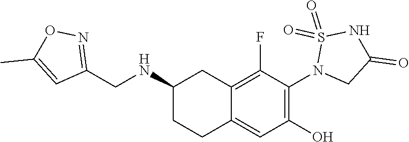 [SMILES: Cc1cc(CN[C@@H]2CCc3cc(O)c(N4CC(=O)NS4(=O)=O)c(F)c3C2)no1] |
| 352 | 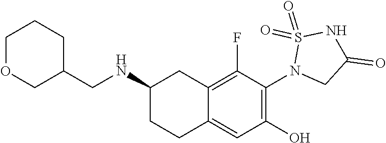 [SMILES: O=C1CN(c2c(O)cc3c(c2F)C[C@H](NCC2CCCOC2)CC3)S(=O)(=O)N1] |
| 353 | 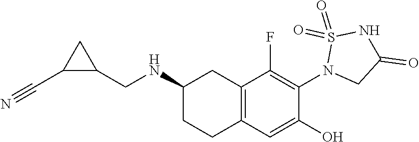 [SMILES: N#CC1CC1CN[C@@H]1CCc2cc(O)c(N3CC(=O)NS3(=O)=O)c(F)c2C1] |
| 354 | 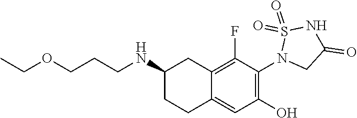 [SMILES: CCOCCCN[C@@H]1CCc2cc(O)c(N3CC(=O)NS3(=O)=O)c(F)c2C1] |
| 355 | 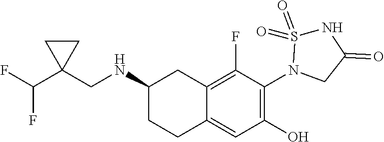 [SMILES: O=C1CN(c2c(O)cc3c(c2F)C[C@H](NCC2(C(F)F)CC2)CC3)S(=O)(=O)N1] |
| 356 | 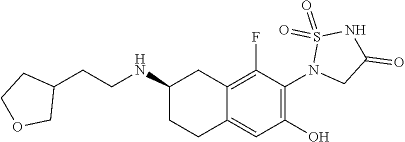 [SMILES: O=C1CN(c2c(O)cc3c(c2F)C[C@H](NCCC2CCOC2)CC3)S(=O)(=O)N1] |
| 357 | 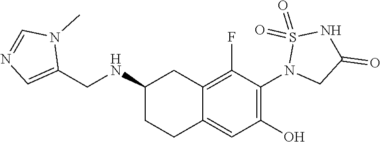 [SMILES: Cn1cncc1CN[C@@H]1CCc2cc(O)c(N3CC(=O)NS3(=O)=O)c(F)c2C1] |
| 358 | 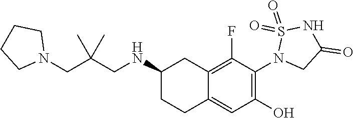 [SMILES: CC(C)(CN[C@@H]1CCc2cc(O)c(N3CC(=O)NS3(=O)=O)c(F)c2C1)CN1CCCC1] |
| 359 | 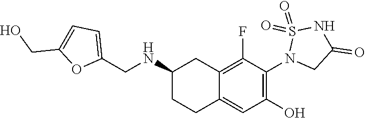 [SMILES: O=C1CN(c2c(O)cc3c(c2F)C[C@H](NCc2ccc(CO)o2)CC3)S(=O)(=O)N1] |
| 360 | 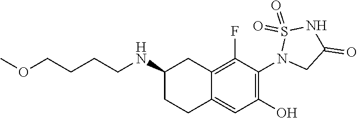 [SMILES: COCCCCN[C@@H]1CCc2cc(O)c(N3CC(=O)NS3(=O)=O)c(F)c2C1] |
| 361 | 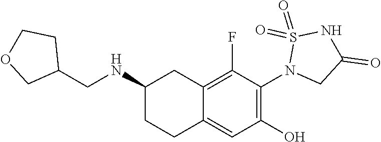 [SMILES: O=C1CN(c2c(O)cc3c(c2F)C[C@H](NCC2CCOC2)CC3)S(=O)(=O)N1] |
| 362 | 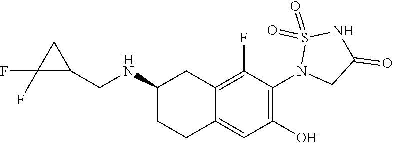 [SMILES: O=C1CN(c2c(O)cc3c(c2F)C[C@H](NCC2CC2(F)F)CC3)S(=O)(=O)N1] |
| 363 | 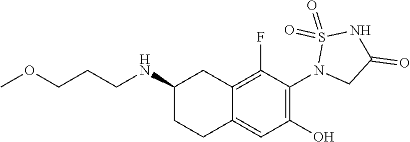 [SMILES: COCCCN[C@@H]1CCc2cc(O)c(N3CC(=O)NS3(=O)=O)c(F)c2C1] |
| 364 | 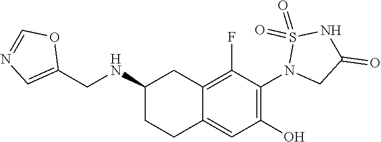 [SMILES: O=C1CN(c2c(O)cc3c(c2F)C[C@H](NCc2cnco2)CC3)S(=O)(=O)N1] |
| 365 | 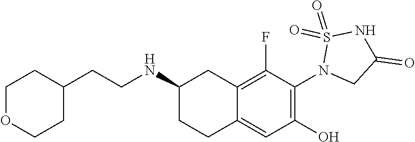 [SMILES: O=C1CN(c2c(O)cc3c(c2F)C[C@H](NCCC2CCOCC2)CC3)S(=O)(=O)N1] |
| 366 | 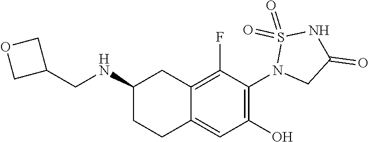 [SMILES: O=C1CN(c2c(O)cc3c(c2F)C[C@H](NCC2COC2)CC3)S(=O)(=O)N1] |
| 367 | 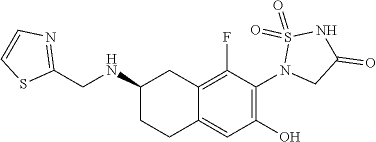 [SMILES: O=C1CN(c2c(O)cc3c(c2F)C[C@H](NCc2nccs2)CC3)S(=O)(=O)N1] |
| 368 | 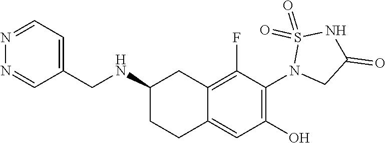 [SMILES: O=C1CN(c2c(O)cc3c(c2F)C[C@H](NCc2ccnnc2)CC3)S(=O)(=O)N1] |
| 369 | 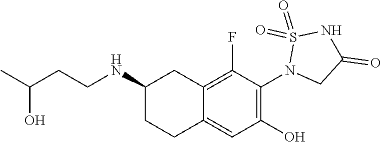 [SMILES: CC(O)CCN[C@@H]1CCc2cc(O)c(N3CC(=O)NS3(=O)=O)c(F)c2C1] |
| 370 | 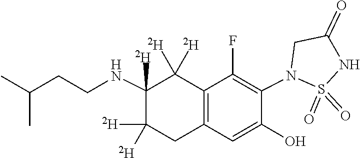 [SMILES: [2H]C1([2H])Cc2cc(O)c(N3CC(=O)NS3(=O)=O)c(F)c2C([2H])([2H])[C@@]1([2H])NCCC(C)C] |
| 371 | 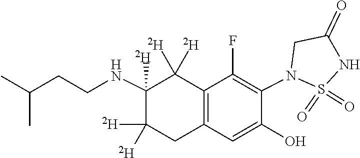 [SMILES: [2H]C1([2H])Cc2cc(O)c(N3CC(=O)NS3(=O)=O)c(F)c2C([2H])([2H])[C@]1([2H])NCCC(C)C] |
| 372 | 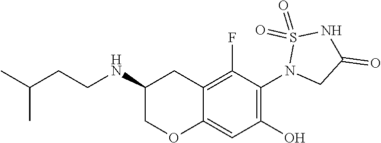 [SMILES: CC(C)CCN[C@@H]1COc2cc(O)c(N3CC(=O)NS3(=O)=O)c(F)c2C1] |
| 373 | 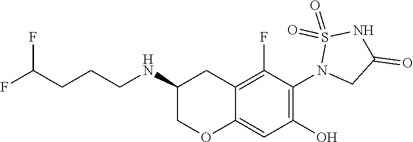 [SMILES: O=C1CN(c2c(O)cc3c(c2F)C[C@H](NCCCC(F)F)CO3)S(=O)(=O)N1] |
| 374 | 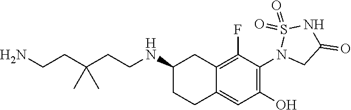 [SMILES: CC(C)(CCN)CCN[C@@H]1CCc2cc(O)c(N3CC(=O)NS3(=O)=O)c(F)c2C1] |
| 375 | 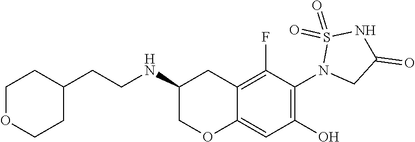 [SMILES: O=C1CN(c2c(O)cc3c(c2F)C[C@H](NCCC2CCOCC2)CO3)S(=O)(=O)N1] |
| 376 | 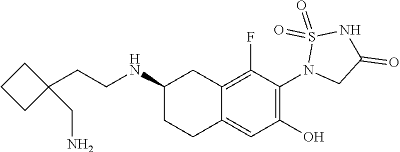 [SMILES: NCC1(CCN[C@@H]2CCc3cc(O)c(N4CC(=O)NS4(=O)=O)c(F)c3C2)CCC1] |
| 377 | 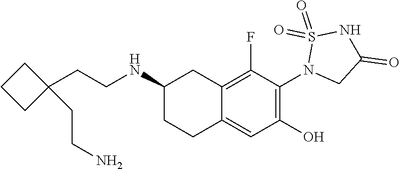 [SMILES: NCCC1(CCN[C@@H]2CCc3cc(O)c(N4CC(=O)NS4(=O)=O)c(F)c3C2)CCC1] |
| 378 | 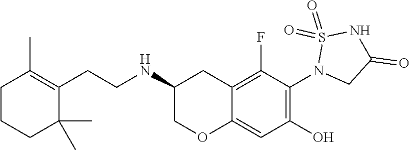 [SMILES: CC1=C(CCN[C@@H]2COc3cc(O)c(N4CC(=O)NS4(=O)=O)c(F)c3C2)C(C)(C)CCC1] |
| 379 | 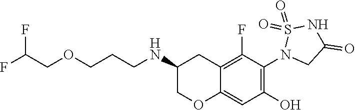 [SMILES: O=C1CN(c2c(O)cc3c(c2F)C[C@H](NCCCOCC(F)F)CO3)S(=O)(=O)N1] |
| 380 | 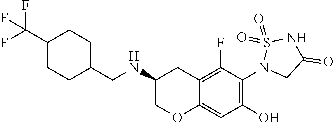 [SMILES: O=C1CN(c2c(O)cc3c(c2F)C[C@H](NCC2CCC(C(F)(F)F)CC2)CO3)S(=O)(=O)N1] |
| 381 | 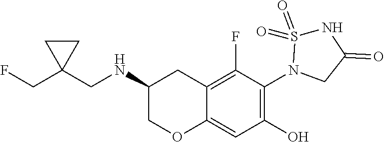 [SMILES: O=C1CN(c2c(O)cc3c(c2F)C[C@H](NCC2(CF)CC2)CO3)S(=O)(=O)N1] |
| 382 | 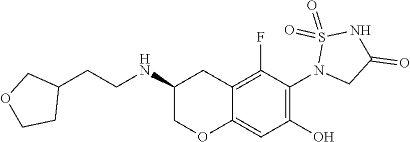 [SMILES: O=C1CN(c2c(O)cc3c(c2F)C[C@H](NCCC2CCOC2)CO3)S(=O)(=O)N1] |
| 383 | 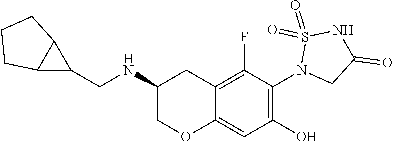 [SMILES: O=C1CN(c2c(O)cc3c(c2F)C[C@H](NCC2C4CCCC42)CO3)S(=O)(=O)N1] |
| 384 | 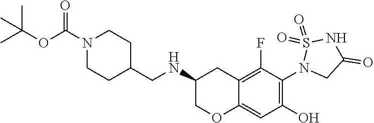 [SMILES: CC(C)(C)OC(=O)N1CCC(CN[C@@H]2COc3cc(O)c(N4CC(=O)NS4(=O)=O)c(F)c3C2)CC1] |
| 385 | 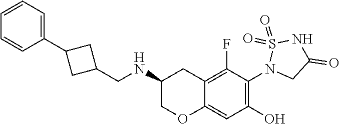 [SMILES: O=C1CN(c2c(O)cc3c(c2F)C[C@H](NCC2CC(c4ccccc4)C2)CO3)S(=O)(=O)N1] |
| 386 | 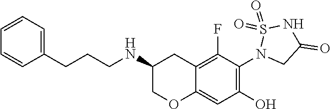 [SMILES: O=C1CN(c2c(O)cc3c(c2F)C[C@H](NCCCc2ccccc2)CO3)S(=O)(=O)N1] |
| 387 | 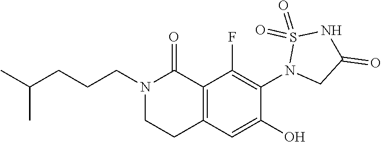 [SMILES: CC(C)CCCN1CCc2cc(O)c(N3CC(=O)NS3(=O)=O)c(F)c2C1=O] |
| 388 | 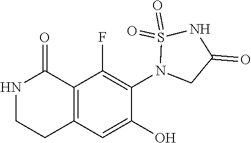 [SMILES: O=C1CN(c2c(O)cc3c(c2F)C(=O)NCC3)S(=O)(=O)N1] |
| 389 | 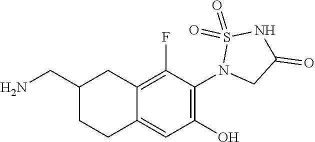 [SMILES: NCC1CCc2cc(O)c(N3CC(=O)NS3(=O)=O)c(F)c2C1] |
| 390 | 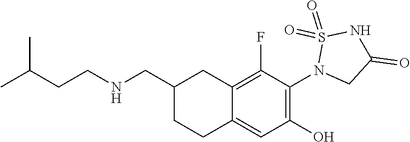 [SMILES: CC(C)CCNCC1CCc2cc(O)c(N3CC(=O)NS3(=O)=O)c(F)c2C1] |
| 391 | 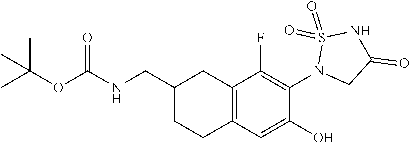 [SMILES: CC(C)(C)OC(=O)NCC1CCc2cc(O)c(N3CC(=O)NS3(=O)=O)c(F)c2C1] |
| 392 | 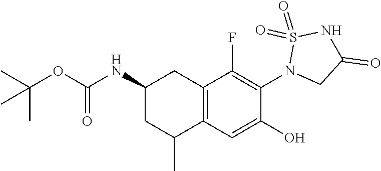 [SMILES: CC1C[C@@H](NC(=O)OC(C)(C)C)Cc2c1cc(O)c(N1CC(=O)NS1(=O)=O)c2F] |
| 393 | 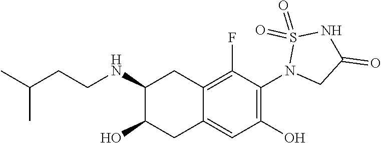 [SMILES: CC(C)CCN[C@H]1Cc2c(cc(O)c(N3CC(=O)NS3(=O)=O)c2F)C[C@H]1O] |
| 394 | 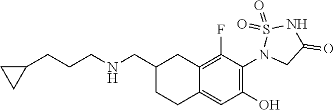 [SMILES: O=C1CN(c2c(O)cc3c(c2F)CC(CNCCCC2CC2)CC3)S(=O)(=O)N1] |
| 395 | 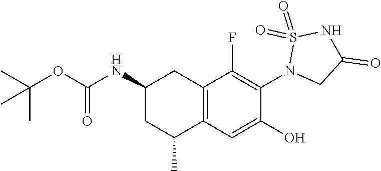 [SMILES: C[C@@H]1C[C@@H](NC(=O)OC(C)(C)C)Cc2c1cc(O)c(N1CC(=O)NS1(=O)=O)c2F] |
| 396 | 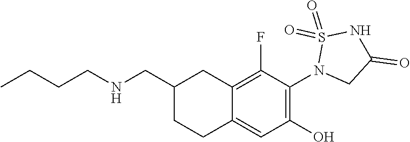 [SMILES: CCCCNCC1CCc2cc(O)c(N3CC(=O)NS3(=O)=O)c(F)c2C1] |
| 397 | 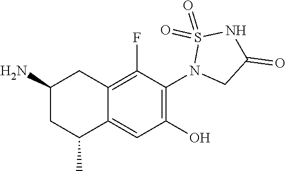 [SMILES: C[C@@H]1C[C@@H](N)Cc2c1cc(O)c(N1CC(=O)NS1(=O)=O)c2F] |
| 398 | 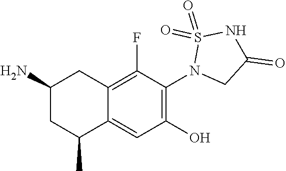 [SMILES: C[C@H]1C[C@@H](N)Cc2c1cc(O)c(N1CC(=O)NS1(=O)=O)c2F] |
| 399 | 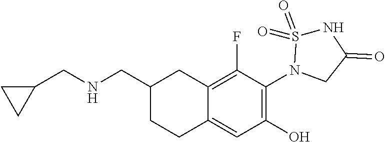 [SMILES: O=C1CN(c2c(O)cc3c(c2F)CC(CNCC2CC2)CC3)S(=O)(=O)N1] |
| 400 | 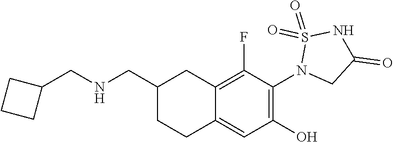 [SMILES: O=C1CN(c2c(O)cc3c(c2F)CC(CNCC2CCC2)CC3)S(=O)(=O)N1] |
| 401 | 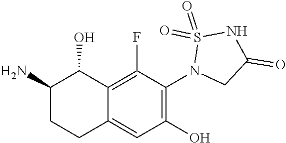 [SMILES: N[C@@H]1CCc2cc(O)c(N3CC(=O)NS3(=O)=O)c(F)c2[C@H]1O] |
| 402 | 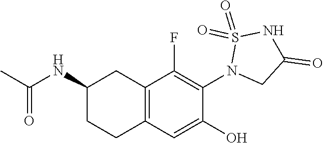 [SMILES: CC(=O)N[C@@H]1CCc2cc(O)c(N3CC(=O)NS3(=O)=O)c(F)c2C1] |
| 403 | 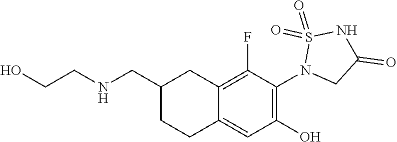 [SMILES: O=C1CN(c2c(O)cc3c(c2F)CC(CNCCO)CC3)S(=O)(=O)N1] |
| 404 | 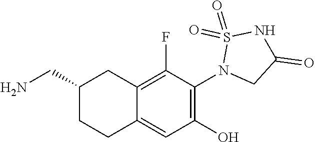 [SMILES: NC[C@H]1CCc2cc(O)c(N3CC(=O)NS3(=O)=O)c(F)c2C1] |
| 405 | 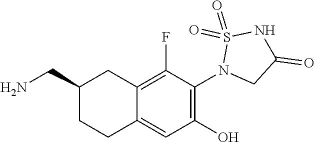 [SMILES: NC[C@@H]1CCc2cc(O)c(N3CC(=O)NS3(=O)=O)c(F)c2C1] |
| 406 | 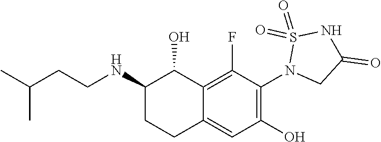 [SMILES: CC(C)CCN[C@@H]1CCc2cc(O)c(N3CC(=O)NS3(=O)=O)c(F)c2[C@H]1O] |
| 407 | 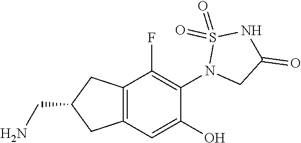 [SMILES: NC[C@H]1Cc2cc(O)c(N3CC(=O)NS3(=O)=O)c(F)c2C1] |
| 408 | 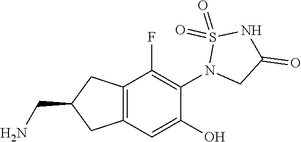 [SMILES: NC[C@@H]1Cc2cc(O)c(N3CC(=O)NS3(=O)=O)c(F)c2C1] |
| 409 | 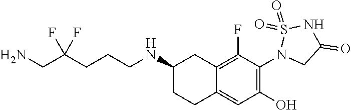 [SMILES: NCC(F)(F)CCCN[C@@H]1CCc2cc(O)c(N3CC(=O)NS3(=O)=O)c(F)c2C1] |
| 410 | 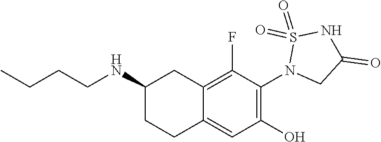 [SMILES: CCCCN[C@@H]1CCc2cc(O)c(N3CC(=O)NS3(=O)=O)c(F)c2C1] |
| 411 | 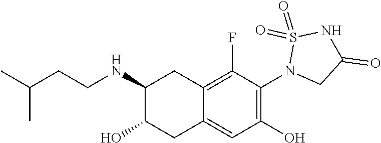 [SMILES: CC(C)CCN[C@H]1Cc2c(cc(O)c(N3CC(=O)NS3(=O)=O)c2F)C[C@@H]1O] |

or a pharmaceutically acceptable salt thereof

65. A pharmaceutically acceptable composition comprising a compound of claim 64 and a pharmaceutically acceptable carrier.

66. The composition of claim 65, wherein the composition is formulated for oral administration.


SEQUENCE LISTING (3 sequences)

SEQ ID NO: 1 - protein, 404 aa, synthetic construct
  REGION 1..404: note = Synthetic Polypeptide
MAMPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRPRLTDTENLYFQSHHHHHHHH

SEQ ID NO: 2 - protein, 13 aa, synthetic construct
  REGION 1..13: note = Synthetic Polypeptide
  SITE 1: note = Xaa - (Oregon Green)-(NH-CH2-CH2-O-CH2-CH2-O-CH2-CO)-L-threonine
  SITE 5: note = Xaa - O-phospho-L-tyrosine
  SITE 13: note = Xaa - L-lysine-NH2
XRDIXETDYYRKX

SEQ ID NO: 3 - protein, 339 aa, synthetic construct
  REGION 1..339: note = Synthetic Polypeptide
MAHHHHHHSSGLVPRGSHMEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHN